SEQUENCE LISTING (915 sequences)

SEQ ID NO: 1 - skipped

SEQ ID NO: 2 - protein, 550 aa, synthetic construct
MEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAV

SEQ ID NO: 3 - RNA, 1843 nt, synthetic construct
  source 1..1843: mol_type = other RNA
acatttgcttctgacacaactgtgttcactagcaacctcaaacagccaccatggaagatgccaaaaacatcaagaagggtccggccccattctacccactggaagatggtaccgcgggcgaacagctgcataaggccatgaagagatacgccctggtccctggaaccatcgcgttcaccgacgcccacattgaagtggacatcacctacgccgaatacttcgagatgtccgtgcggctggccgaagccatgaagcgctacggcctgaacacgaaccataggatcgtggtctgctccgaaaacagcctgcagtttttcatgccggtgctcggagccctgtttatcggcgtggcggtcgcaccagcaaacgacatctacaatgagcgggaactactgaactcgatgggtatttcccaacccactgtggtgttcgtcagcaaaaagggcctgcaaaagatcctgaacgtgcagaagaagctgcccatcatccagaagatcattattatggacagcaagacggattaccaaggcttccagtcgatgtacaccttcgtgacctcgcatctgccaccgggtttcaacgaatacgacttcgtgcccgaatcgtttgaccgcgacaagactattgccttgatcatgaacagcagcggtagtactggtttgcctaagggggtggccctgccacaccggaccgcctgcgtgagattctcgcacgcgcgcgatcccatattcggaaatcagatcatccctgacacggctatcctgtccgtggtcccattccaccacggcttcggcatgttcaccactctcggatacctgatttgcggattccgggtggtgctgatgtaccgcttcgaagaggagctgtttctccggagccttcaggactacaaaatccagagcgccctcctggtgcccaccctgttctccttcttcgctaagtctaccttgattgacaaatacgacctctcgaacctccacgagatcgcatccggtggtgccccattgtcgaaggaagtcggagaagcagtggcgaagcggttccacctcccgggcattcggcagggttacgggctgactgaaaccacctcagcgatcctcattactcctgagggggacgacaagccgggcgccgtgggaaaggttgtgccgttcttcgaggccaaggtcgtcgacctggataccggaaagaccctgggagtcaaccaaaggggggagctgtgtgtgcgcggacctatgattatgtccggatacgtcaacaaccctgaggccactaacgccctgatcgacaaggacggttggctgcactccggagatatcgcatactgggacgaggacgaacacttcttcattgtggatcggttgaagtccctgatcaagtacaagggctatcaagtggcaccggccgaacttgaatcgatccttctgcaacaccccaacatcttcgatgccggcgtcgccgggctgcccgacgacgacgcgggagagctgccggctgcggtggtggtgcttgagcacggcaaaaccatgactgaaaaagagatcgtggactacgtggcgtcccaagtcaccaccgcgaagaagctcaggggcggcgtggtgtttgtggatgaggtgcccaaggggctcaccggaaagttggacgcccgcaagattcgcgagattctgattaaggccaagaaggggggaaagattgccgtgtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 4 - RNA, 628 nt, synthetic construct
  source 1..628: mol_type = other RNA
acatttgcttctgacacaactgtgttcactagcaacctcaaacagacaccatggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 5 - protein, 567 aa, unidentified
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 6 - RNA, 1894 nt, synthetic construct
  source 1..1894: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgctaccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccaccactcgaacgagcagggatcgggctatgccgccgacaaggagagcacccagaaggccatcgacggggtcaccaacaaggtcaactccatcattgacaagatgaatacccagttcgaagccgtgggccgcgaattcaacaacctggaacgacggatcgagaacctgaacaagaagatggaggacggcttcctggacgtgtggacctacaatgccgaactgctcgtgctgatggagaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcctttccatctactccacggctgcgagctcgttggcccttgcgatcatgatggccggcctttcgctgtggatgtgctccaacggtagcctgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 7 - protein, 584 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESVGTYQIGIVAGLAVLAVVVIGAVVAAVMCRRKSSGGKGGSYSQAACSDSAQGSDVSLTA

SEQ ID NO: 8 - RNA, 1945 nt, synthetic construct
  source 1..1945: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgctaccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccaccactcgaacgagcagggatcgggctatgccgccgacaaggagagcacccagaaggccatcgacggggtcaccaacaaggtcaactccatcattgacaagatgaatacccagttcgaagccgtgggccgcgaattcaacaacctggaacgacggatcgagaacctgaacaagaagatggaggacggcttcctggacgtgtggacctacaatgccgaactgctcgtgctgatggagaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcgggattgtggccggactggctgtgttggccgtggtggtcatcggcgccgtcgtggcggccgtgatgtgccggcgcaagtcctccgggggcaaagggggaagctattcccaagccgcctgctctgactccgcccaggggtcggatgtgtccctgaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 9 - protein, 535 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 10 - RNA, 1798 nt, synthetic construct
  source 1..1798: mol_type = other RNA
acatttgcttctgacacaactgtgttcactagcaacctcaaacagccaccatggaactcctcatcctgaaagccaacgccatcaccacaattctcactgctgtgaccttctgttttgcctcgggacagaacatcaccgaggagttctaccagtccacatgctccgctgtgtcgaagggctacctgtcggcgctgaggactggctggtacacctccgtgattaccatcgaactgtcaaacatcaaggagaacaagtgcaacggtactgacgcgaaggtcaagctgattaagcaggaattggacaagtacaagaacgccgtgactgagctccagctgctgatgcagtcaacccctgcgacgggaagcgggtccgcaatctgctccggagtggccgtgtgcaaagtgctgcatctggagggcgaagtcaacaagatcaagtcggccctgctgagcaccaacaaggccgtggtgtccttgagtaacggagtctccgtcctgaccttcaaagttctggacctgaagaattacatcgataagcagctcctgcccatcctcaacaagcagagctgctccatttcgaacatcgaaactgtgatcgagtttcagcaaaagaacaaccggctgctggaaatcaccagagagtttagcgtgaacgcaggagtgactacccccgtgtctacctacatgctaaccaactcagaactgcttagcttgatcaatgacatgccgatcactaacgatcagaagaagcttatgtccaacaacgtgcaaatcgtgcgccagcaatcctactcgattatgtgcattatcaaggaagaggtgctggcctatgtggtgcagctcccgctgtacggcgtgattgacactccttgctggaagctgcacacctcccccctgtgtaccacgaataccaaggaggggtccaatatctgcctgacccggaccgaccgcggatggtactgcgacaacgccggttcggtgtccttcttcccacaagccgagacttgcaaagtccagtccaatcgcgtgttctgcgacactatgaactcccggaccttgccgtccgaagtgaacctgtgtaacgtggacatcttcaaccctaaatacgactgcaagattatgacttccaagaccgacgtcagcagctcagtgatcaccagcctgggcgccatcgtgtcgtgttatggaaagaccaagtgcactgcgtccaacaagaacagaggcatcatcaagaccttcagcaacggctgcgattacgtcagcaacaagggggtggataccgtgtccgtgggaaacactctctactgtgtgaacaagcaggagggaaagtccctctacgtcaagggcgaaccgatcatcaacttctacgatcccctggtgttcccatccgacgagttcgacgcctccatttcacaagtcaacgaaaagatcaaccagtctctggcgttcatccggaagtcggatgaactccttcacaacgtcaatgccggaaagtccactactaacatcatgattaccaccatcatcattgtcattattgtgatacttctgagcctcatcgccgtgggtctgctgttgtactgcaaagctcgctccacgcctgtgaccctgtccaaggatcagctcagcgggattaacaacatcgccttctcaaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 11 - skipped

SEQ ID NO: 12 - protein, 566 aa, Alphainfluenzavirus influenzae
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIATRPKVRDQAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 13 - RNA, 1891 nt, synthetic construct
  source 1..1891: mol_type = other RNA
acatttgcttctgacacaactgtgttcactagcaacctcaaacagccaccatgaaagccatcctcgtcgtaatgctgtacaccttcactaccgccaatgccgacaccctgtgcatcggataccatgccaacaactccaccgacaccgtggacactgtgctggagaagaacgtgaccgtgacccactcggtgaacttgctggaagataagcacaacggaaagctgtgcaagctcaggggggtcgcacctctgcatctgggacagtgcaacatcgctggctggatcctgggaaacccggagtgcgagagcctgtccacggccagaagctggagctacatcgtggaaacttcgaattcggataacggcacctgttatcccggggatttcattaactacgaggaactgcgcgagcagctctcatcggtgtcctccttcgaacgcttcgaaatcttccctaaaacctcatcctggcctaaccacgacagcgacaacggcgtgactgcagcctgctcccacgccggggcgcggtcgttctacaagaacctcatctggctggtcaagaagggaaagtcctatcctaaaatcaaccagacctacatcaacgacaagggaaaggaagtgttggtgctctgggggatccatcatcccccgaccatcaccgaccaggaaagcctgtaccagaacgccgatgcctacgtgtttgtcggaacctcgcgctactcaaagaagtttaagccggagattgccacccggccgaaagtccgggaccaggctggacggatgaactactactggaccctggtggaaccaggggacaagatcacattcgaagcgaccggaaacctggtggcacctagatacgcgttcaccatggagaaggaggccggttccggtatcatcattagcgacaccccggtgcacgactgcaacgctacttgccaaactcccgaaggcgccatcaataccagccttcccttccaaaacgtgcacccaattaccatcggaaagtgtcccaaatacgtgcggtccactaagttgaggttggcgactgggctgaggaatgtgcccagcattcagtcccggggactgttcggagccatcgccggtttcatcgagggcggctggactggaatggtggacggttggtacggctaccaccaccaaaacgaccagggctcaggctacgcggctgaccttaagtccactcagaatgcgatcgataagatcacgaacaaggtcaactccgtgattgaaaagatgaacactcagtttaccgccgtcggaaaggaattcaaccatctggagaagcgcattgaaaacctgaacaagaaggtcgatgacggtttcctcgacgtgtggacctacaacgcggagctgcttgtgctgctcgagaacgagcggaccctggactatcacgatagtaacgtgaagaacctgtacgagaaggtccgccaccaactgaagaacaacgcaaaggaaatcggcaacggctgtttcgagttctaccacaaatgcgataacacttgcatggaatccgtgaagaatggcacctacgactacccgaagtactccgaagaggccaagctcaaccgagagaagattgatggagtgaagctggactctactagaatctaccagatacttgccatctactccaccgtggcctcaagcctggtgctcgtggtgtccctgggagccatttccttctggatgtgctccaacgggtcgctccagtgtcgcatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 14 - protein, 566 aa, Alphainfluenzavirus influenzae
MKTIIALSNILCLVFAQKIPGNDNSTATLCLGHHAVPNGTIVKTITNDRIEVTNATELVQNSSIGEICGSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTSLNNIYPAQNVTMPNKEQFDKLYIWGVHHPDTDKNQISLFAQSSGRITVSTKRSQQAVIPNIGSRPRIRGIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 15 - protein, 567 aa, synthetic construct
MENIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCFPGSLNDYEELKHMLSRINHFEKIQIIPKSSWPNHETSLGVSAACPYQGAPSFFRNVVWLIKKNDAYPTIKISYNNTNREDLLILWGIHHSNNAEEQTNLYKNPITYISVGTSTLNQRLAPKIATRSQVNGQRGRMDFFWTILKPDDAIHFESNGNFIAPEYAYKIVKKGDSTIMKSGVEYGHCNTKCQTPVGAINSSMPFHNIHPLTIGECPKYVKSNKLVLATGLRNSPLREKRRKRGLFGAIAGFIEGGWQGMVDGWYGYHFSNEQGSGYAADKESTQKAIDGVTNMVNSIIDKMNTQFEAVGMEFNNLERRIENLNKKMEDGFIDVWTYNAELLVLMLNERTLDFHDSNVKNLYDKVRLQLRDNAKELGNGCFEFYHKCDNECMESVRNGTYDYPQYSEEARLKREEISGVKLESVGTYQILSIYSTAASSLALAIMMAGLSLWMCSNGSLQCRICI

SEQ ID NO: 16 - RNA, 1894 nt, synthetic construct
  source 1..1894: mol_type = other RNA
acatttgcttctgacacaactgtgttcactagcaacctcaaacagccaccatggagaacatcgtgctcttgctggcaatcgtgtccctggtcaagtccgatcagatctgcatcggctaccacgcaaacaactccaccgaacaagtggacactatcatggaaaagaacgtgacggtcacacacgcccaggacattctggaaaagacccacaatgggaaactgtgtgacctcaacggcgtgaagccgctgatcctcaaggattgctccgtggctggatggctcctgggcaaccctatgtgcgatgagttcattcgggtgcccgagtggtcgtacattgtggagagggccaacccggccaatgacctgtgcttccccggctcgctgaacgattacgaagaactcaagcacatgctgtcccgcatcaaccatttcgagaagatccagatcattccgaagtccagctggcctaaccacgaaacttccctgggagtgtctgctgcctgcccataccaaggcgccccctcatttttccggaacgtcgtgtggctcatcaagaagaacgatgcgtaccccactatcaagatcagctacaacaacaccaaccgcgaggatctgctgattctctggggaattcaccattccaacaacgctgaagaacagaccaacctgtacaaaaaccctatcacttacatctccgtgggtacctccaccctcaatcagcgcttggcgccgaagatcgcgaccagaagccaagtcaacggacagcggggaagaatggacttcttctggactatactgaagcctgacgatgcaatccacttcgaatccaacggaaacttcatcgcccctgagtatgcctacaagatcgtgaagaagggcgacagcaccatcatgaagtcaggagtggagtatggacactgtaacactaagtgccagacccccgtcggggccattaacagctccatgccgttccacaacattcaccccctgactattggagagtgtccgaaatacgtgaagagtaacaagttggtgctggccactggcctgcgcaactcgccgcttcgggagaagaggaggaagagaggactgtttggggcgattgcaggattcattgagggaggttggcaggggatggtggacggttggtacggttaccacttctcgaacgagcagggatcgggctatgccgccgacaaggagagcacccagaaggccatcgacggggtcaccaacatggtcaactccatcattgacaagatgaatacccagttcgaagccgtgggcatggaattcaacaacctggaacgacggatcgagaacctgaacaagaagatggaggacggcttcatcgacgtgtggacctacaatgccgaactgctcgtgctgatgctgaatgagcggaccctcgacttccatgactcaaacgtgaagaacctgtacgacaaagtccggctgcaactgcgggacaatgccaaggaactggggaacggatgctttgagttctaccataagtgcgataacgagtgcatggaatcagtgcggaacggaacctacgactacccacagtactccgaggaagcccgcctgaagagagaagaaatctccggcgtgaaactcgaaagcgtggggacctaccagatcctttccatctactccacggctgcgagctcgttggcccttgcgatcatgatggccggcctttcgctgtggatgtgctccaacggtagcctgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 17 - RNA, 1939 nt, synthetic construct
  source 1..1939: mol_type = other RNA
acatttgcttctgacacaactgtgttcactagcaacctcaaacagccaccatgaaggccattattgtgctcctcatggtcgtgacttcaaacgcggacaggatctgcactgggattacctcctcgaactccccgcacgtggtcaaaactgcaactcaaggagaagtcaacgtgaccggggtcatacccctcactacgaccccgaccaaatcccatttcgcgaaccttaagggtaccgagactcggggaaaactctgcccgaagtgtctgaactgtactgacctggatgtggcgctcgggcgcccaaagtgcacgggaaagattccgtccgcacgcgtcagcatcttgcacgaagtcaggcccgtgacctccggatgctttccaattatgcacgaccggacgaagatccgccagctcccgaacctcttgaggggttatgagcacgtccgcctctcgactcataacgtcattaacaccgaggatgcgccgggaggaccctacgaaattggtacctcgggatcatgccttaacatcaccaacgggaagggattctttgccaccatggcctgggcggtgcccaagaacaagaccgccaccaaccctcttaccatcgaagtgccatacatctgcaccgaggaagaggaccagatcactgtgtggggtttccattccgacgacgaaactcagatggcccgcctctacggggactccaagccgcaaaagttcacctcgtccgccaatggagtgactacccactacgtgtcccaaattggagggttcccgaatcaaactgaggatggtggactgccccagagcggccggattgtggtggattacatggtccagaaaagcggcaagaccggcaccatcacctaccagcggggaatcctgctgcctcagaaggtgtggtgtgcttccggcaagagcaaggtgatcaagggcagcctgcctctgatcggcgaagccgactgcctgcacgagaagtacggcggcctgaacaagagcaagccttactacacaggcgaacatgccaaggccatcggcaactgccccatctgggtgaagacccccctgaagctggccaacggaaccaagtacagacctcccgccaagctgctgaaggaacggggcttcttcggagccatcgccggcttcctggaaggaggctgggagggcatgattgccgggtggcacggctacacctctcatggagcacacggggtggccgtggctgctgacctgaagtctacacaggaggccatcaacaagatcaccaagaacctgaactctttgtctgagctggaggtgaaaaacctgcagagactgtcaggcgccatggacgaactgcacaatgagatcctggaactggatgaaaaggtggacgacctgagagccgacaccatcagctcccagatcgaactggctgtgctgctgagcaacgagggaatcatcaattccgaggacgaacacctgctggccctggagcggaagctgaagaagatgctgggcccttctgccgtggaaatcggcaacggctgcttcgaaaccaagcacaagtgcaaccagacctgcctggaccggattgcagccggcacctttgacgccggagagttctccttgccgactttcgactcgctcaacatcactgcggcctcgcttaacgacgatggtctggataaccacactatccttttgtattactccaccgcggcgtcgtccctcgccgtgaccctcatgatcgctattttcgtggtgtacatggtgtcccgcgataacgtgtcctgttcaatctgcctgtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 18 - protein, 566 aa, unidentified
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKDESFNWTGVKQNGTSSACKRGSSNSFFSRLNWLTSLNNIYPAQNVTMPNKEQFDKLYIWGVHHPDTDKNQFSLFAQSSGRITVSTKRSQQAVIPNIGSRPRVRDIPSRISIYWTIVKPGDILLINSTGNLIAPRGYFKIRSGKSSIMRSDAPIGECKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 19-31 - skipped (13 entries)

SEQ ID NO: 32 - protein, 17 aa, Alphainfluenzavirus influenzae
MKAILVVMLYTFTTANA

SEQ ID NO: 33 - protein, 36 aa, Alphainfluenzavirus influenzae
LAIYSTVASSLVLVVSLGAISFWMCSNGSLQCRICI

SEQ ID NO: 34 - skipped

SEQ ID NO: 35 - protein, 28 aa, Homo sapiens
MAAPGSARRPLLLLLLLLLLGLMHCASA

SEQ ID NO: 36 - protein, 35 aa, Homo sapiens
LIPIAVGGALAGLVLIVLIAYLVGRKRSHAGYQTI

SEQ ID NO: 37 - protein, 469 aa, Alphainfluenzavirus influenzae
MNPNQKIITIGSVCMTIGTANLILQIGNIISIWVSHSIQIGNQSQIETCNKSVITYENNTWVNQTFVNISNTNSAARQSVASVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGTNWLTIGISGPDSGAVAVLKYNGIITDTIKSWRNKILRTQESECACVNGSCFTIMTDGPSDGQASYKIFRIEKGKIIKSVEMKAPNYHYEECSCYPDSSEITCVCRDNWHGSNRPWVSFNQNLEYQMGYICSGVFGDNPRPNDKTGSCGPVLSNGANGVKGFSFKYGNGVWIGRTKSISSRKGFEMIWDPNGWTGTDNKFSKKQDIVGINEWSGYSGSFVQHPELTGLNCIRPCFWVELIRGRPEENTIWTSGSSISFCGVDSDIMGWSWPDGAELPFTIDN

SEQ ID NO: 38 - protein, 469 aa, Alphainfluenzavirus influenzae
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDLDKCYQFALGQGTTLNNVHSNNTVRDRTPYRTLLMNELGVPFHLGTKQVCIAWSSSSCHDGKAWLHVCITGDDKNATASFIYNGRLVDSVVSWSNDILRTQESECVCINGTCTVVMTDGNATGKADTKILFIEEGKIVHTSKLSGSAQHVEECSCYPRYPGVRCVCRDNWKGSNRPIIDINIKDHSIVSRYVCSGLVGDTPRKSDSSSSSHCLNPNNEKGDHGVKGWAFDDGNDVWMGRTINETSRLGYETFKVVEGWSNPKSKLQINRQVIVDRGDRSGYSGIFSVEGKSCINRCFYVELIRGRKEETEVLWTSNSIVVFCGTSGTYGTGSWPDGANLSLMHI

SEQ ID NO: 39 - protein, 466 aa, Betainfluenzavirus influenzae
MLPSTIQTLTLFLTSGGVLLSLYVSASLSYLLYSDILLKFSQTEITAPTMPLDCANASNVQAVNRSATKGATLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDEGSGGIKGGFVHQRMKSKIGRWYSRTMSKTERMGMGLYVKYGGDPWADSDALVFSGVMISMKEPGWYSFGFEIKDKKCDVPCIGIEMVHDGGKETWHSAATAIYCLMGSGQLLWDTITGVDMAL

SEQ ID NO: 40-400 - skipped (361 entries)

SEQ ID NO: 401 - protein, 1123 aa, synthetic construct
MRVTAPRTLILLLSGALALTETWAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALGIVAGLAVLAVVVIGAVVAAVMCRRKSSGGKGGSYSQAACSDSAQGSDVSLTA

SEQ ID NO: 402 - RNA, 3562 nt, synthetic construct
  source 1..3562: mol_type = other RNA
acatttgcttctgacacaactgtgttcactagcaacctcaaacagccaccatgcgggtcactgcccccagaaccttgattttactactgtcgggagccctcgctctgactgaaacatgggccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcggcatcgtggcgggcctcgctgtcctcgccgtggtggtgatcggagccgtcgtcgccgctgtgatgtgccgccgcaagtccagcggcggaaaggggggcagctatagccaggcagcctgttccgactccgcgcaggggtcggatgtgtccttgaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 403 - protein, 1109 aa, synthetic construct
MAAPGSARRPLLLLLLLLLLGLMHCASAAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGGGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGAQIYQAVSAQAAAIHGPGPGPSPSMGRDIKVQFQSGPGPGGINTIPIAINEAEYVGPGPGAAFQGAHARFVAAAAGPGPGAGWLAFFRDLVARGLGPGPGASIIRLVGAVLAEQHGPGPGMSFVTTQPEALAAAAGPGPGMHVSFVMAYPEMLAAGPGPGAYGSFVRTVSLPVGAGPGPGLENDNQLLYNYPGALLIPIAVGGALAGLVLIVLIAYLVGRKRSHAGYQTI

SEQ ID NO: 404 - RNA, 3520 nt, synthetic construct
  source 1..3520: mol_type = other RNA
acatttgcttctgacacaactgtgttcactagcaacctcaaacagccaccatggccgctcctggatccgcccgcagaccgttgttgctcctgttactactgctcttacttggactcatgcactgcgcctccgccgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcacaaatcatgtacaactacccggcaatgctgggccacgctggagacatggccggctacgccggcacgctgcagtcactcggtgccgagatcgccgtcgagcaggcggcactgcagagcgcttggcagggagacactggcattacctaccaagcgtggcaggctcagtggaatcaagctatggaagatctggtccgcgcgtaccacgccatgtcctccactcacgaagccaacaccatggctatgatggccagggacactgccgaggcagccaaatgggggggaggaccggggccgggaaccataaactaccagtttggcgacgtcgacgcccacggagccatgatcagggcgcaggccgggtcgctggaagcagaacaccaggccatcatctccgatgtgctgaccgcctccgacttttggggaggagccggttcggctgcctgccaagggttcatcacacaattgggaaggaacttccaggtcatctacgagcaggccaatgcacacggtcaaaaggtccaagcggcgggcaacaacatggcccagactgactcggccgtgggcagcagttgggccggtcccggccctggccctgtcggggggcagtcatccttctactccgactggtactcccccgcctgcggaaaggccggctgtcagacctataagtgggaaaccttcctgacctccgaactcccgcagtggctctcggcgaatcgggcagtgaagcccaccgggtcggcggctattggactgtccatggccggttccagcgcgatgatcctggccgcctatcacccccagcaattcatctacgctggctccctgtccgccctgcttgacccatcgcaggggatgggtggaccgggccctggggcccagatctaccaagcagtgtcggcccaagccgcagcgatccatggaccaggaccgggaccgtccccgagcatgggccgggatattaaggttcagttccagtccggacccggcccggggggtattaacaccattccgattgccattaatgaggcagaatacgtcggccctggtcccggcgccgcgttccaaggagcccacgcacgattcgtggcggcagctgcgggacccgggcctggtgccggttggctggcttttttccgcgacctggtggccagaggacttggacccggtccgggcgccagcattatcaggctggtcggggccgtgctggcggaacagcatggacctggcccgggaatgagcttcgtgaccacccaacctgaggcgcttgccgcggcggcaggccctggacccgggatgcatgtgtcttttgtgatggcctacccggaaatgctggccgcgggtccgggccccggagcctacggatcattcgtgcgcactgtgtccctccccgtgggagccggacctggaccggggttggagaatgataaccagctgctctacaactatccgggggccctcctgattcctatcgccgtgggcggtgccttggccggcctggtgctgatcgtcctgattgcctatctcgtggggaggaagagatcgcatgccggctaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 405 - protein, 1109 aa, synthetic construct
MRVTAPRTLILLLSGALALTETWATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVGIVAGLAVLAVVVIGAVVAAVMCRRKSSGGKGGSYSQAACSDSAQGSDVSLTA

SEQ ID NO: 406 - RNA, 3520 nt, synthetic construct
  source 1..3520: mol_type = other RNA
acatttgcttctgacacaactgtgttcactagcaacctcaaacagccaccatgcgcgtgactgcgcctaggaccctgattctgctcctgtcgggtgccctcgctctgaccgaaacctgggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcggccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcggccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcggcattgtggccggactggccgtgttggccgtcgtcgtgatcggcgctgtggtcgccgccgtgatgtgccgaagaaagtccagcggtggaaaggggggatcctactcacaagcagcctgcagcgactcagcgcaggggtcggacgtgtcactgacagcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 407 - protein, 1095 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGPGPGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGPGPGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGPGPGTEQQWNFAGIEAAASAIQGNVTSIHSLLDEGKQSLTKLAAAWGGSGSEAYQGVQQKWDATATELNNALQNLARTISEAGQAMASTEGNVTGMFAGPGPGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGPGPGSFVMAYPEMLAAAADTLQSIGATTVASNAAAAAPTTGVVPPAADEVSALTAAHFAAHAAMYQSVSARAAAIHDQFVATLASSASSYAATEVANAAAASGPGPGTINYQFGDVDAHGAMIRAQAGSLEAEHQAIISDVLTASDFWGGAGSAACQGFITQLGRNFQVIYEQANAHGQKVQAAGNNMAQTDSAVGSSWAGPGPGPVGGQSSFYSDWYSPACGKAGCQTYKWETFLTSELPQWLSANRAVKPTGSAAIGLSMAGSSAMILAAYHPQQFIYAGSLSALLDPSQGMGGPGPGTVPPPVVAANRAELAVLAGPGPGMVDPVVVAANRSAFVQLVLIPIAVGGALAGLVLIVLIAYLVGRKRSHAGYQTI

SEQ ID NO: 408 - RNA, 3478 nt, synthetic construct
  source 1..3478: mol_type = other RNA
acatttgcttctgacacaactgtgttcactagcaacctcaaacagccaccatggcggctccgggaagcgcacgacgcccgctgctgctcttgttgctgctcctcctgctgggtctgatgcactgcgcctcggccaccgccgcgagcgacaattttcagttaagccagggtggccagggatttgccatcccgatcggacaggccatggctatcgcgggccagatcagatccggaggcggatccccgactgtacacatcggtccgaccgccttcttgggactgggagtggtggacaacaacgggaatggcgcacgggtgcagagggtcgtgggctcggcaccagccgcctctctgggaatcagcaccggggacgttatcaccgctgtggatggtgcccccatcaattccgccacggcgatggccgatgccctgaacggccatcaccccggggatgtgatatccgtgacctggcagaccaagtccggcggtacaaggaccggcaacgtcactctggccgagggtcctcctgcgggccctggtcctggtgctgagatgaaaaccgacgccgcgaccctggcccaggaagccggaaacttcgagcggatttccggagatcttaagactcagatcgaccaggtcgaatccaccgccggatcgctccagggacagtggagaggcgccgcgggaaccgcggcccaggccgccgtcgtgcggttccaagaggccgccaacaagcagaagcaggaactggacgaaatttccactaacattcgccaagctggcgtgcagtactcgagagccgatgaagaacagcagcaagccctctcctcacaaatgggtttcggacctgggcccggcgtggacttcggagccctgccacctgaaatcaactccgcccggatgtacgcaggacctggatccgccagcctggtggcagccgcgcagatgtgggatagcgtggcctccgacctgttcagcgcggcttcagctttccaaagcgtggtctggggactgactgtgggatcctggatcggatcaagcgcaggcctcatggtggccgcggcgtccccttacgtggcctggatgagcgtgaccgccggccaggcagaactgaccgcagcccaagtccgcgtggccgcagccgcctacgagactgcctacggtctgacggtgccgccgccagtgatcgccgagaacagagcagagctcatgatcctcatcgcgaccaacctactgggccagaacactccggcgattgccgtgaacgaagccgaatatggggagatgtgggctcaggacgctgcagccatgttcggatatgcagccgcgactgctaccgccaccgccactctgctgccgttcgaggaggcccccgaaatgacttccgcgggaggcctgttggagcaagctgccgcggtggaggaagctagcgacaccgccgcagccaaccagctcatgaacaacgtgccacaggccctgcagcagctggcccagcccactcaagggaccaccccgagctcaaagctgggcggtctgtggaaaaccgtgtccccccaccgctcgcccatttccaacatggtgtcaatggcgaacaaccacatgggtcctggacctggaacggagcagcagtggaacttcgccgggatcgaagccgccgcctcggctatccaagggaatgtcacctcgatccattcccttctggacgaaggaaagcagtccctgaccaagcttgcggcagcctggggcggaagcggcagcgaagcctaccagggcgtgcagcaaaaatgggacgcaaccgccactgagctgaacaacgccctccaaaacctggctagaactatttccgaggccggacaggctatggccagcaccgagggcaacgtgaccgggatgttcgctggccctgggccgggcacctcgcggttcatgaccgatcctcatgctatgcgggatatggctggacggttcgaggtgcacgcccaaactgtggaggacgaggcccgccggatgtgggccagcgcgcagaacatctcgggggccggctggtccggaatggccgaagccacctccctcgataccatgacccagatgaaccaggccttccggaacattgtgaacatgctgcatggagtgcgggacggcctggtccgggacgccaacaattacgagcagcaggagcaggcctctcagcaaatcctgtccagcgggccggggcctggatcattcgtgatggcctaccctgagatgctggcagccgcggccgacaccctgcaatccattggcgccactaccgtggcatcgaacgcggcagccgccgcaccgactaccggcgtggtgccgcccgccgctgatgaagtgtccgcccttactgccgcccacttcgccgcccacgccgcgatgtaccagtccgtgtccgcgcgcgccgccgctatccacgatcagttcgtcgccacactggccagctcagcctcctcatacgccgctactgaagtggcaaacgcagccgctgcatccggcccgggacccggaacgatcaactaccagtttggcgacgtggacgcgcacggggcgatgatcagggctcaagcggggtccctcgaggccgaacaccaagcgattatctcggatgtcctcaccgcgtccgacttctggggtggagccggaagcgccgcctgccaaggtttcatcacccaactgggtcgcaactttcaagtcatctacgaacaggccaacgcccacggacagaaggtccaagccgccggaaacaacatggcacagaccgactccgcggtcggatcgagctgggccggacccgggccaggtcccgtgggcggacagagctcgttctattctgactggtactcacccgcatgtggaaaggccgggtgccagacctacaaatgggaaaccttcttgacttccgagctgcctcagtggttgtccgccaatcgggccgtgaagccgaccggctcggcggccatcggcctctccatggccggctcgtccgcgatgattcttgccgcctaccacccgcagcagttcatctacgccgggtccctctcggcactgctggaccccagtcagggaatgggcggacccggaccgggaactgtcccgcccccggtggtggctgctaaccgcgccgaacttgccgtgctggccggtccagggccgggaatggtggacccagtggtggtggcagcgaacagatccgcattcgtgcaactggtcctgattcctattgcggtcggcggggctctcgccgggctcgtgctcattgtcctgattgcctacctggtcggacgcaagaggtcccatgcgggctaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 409 - protein, 1179 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNEPSSQSTVPIVGIVAGLAVLAVVVIGAVVAAVMCRRKSSGGKGGSYSQAACSDSAQGSDVSLTA

SEQ ID NO: 410 - RNA, 3730 nt, synthetic construct
  source 1..3730: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggaggttctggaggaggcggctccggtggcgcggagatgaaaaccgatgcggccaccctggcacaggaggctggaaacttcgaaagaatcagcggcgacctcaagactcagatcgaccaggtcgaatcgactgccggaagcctccagggccagtggaggggagccgctggaactgcggcgcaggcggccgtggtcagattccaggaggccgccaacaagcagaagcaggaactggatgaaatttccactaacatcagacaggctggggtgcaatactcccgcgcggacgaagaacagcagcaggccctgtccagccaaatgggattcggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcaccgagcagcagtggaacttcgccggaatcgaggccgcagcgagcgcaatccagggcaatgtcacctccattcactccctgctggatgagggcaaacagtccctgactaagcttgccgcagcgtggggaggttccggctccgaagcctaccagggcgtgcagcagaaatgggacgctactgctaccgaactgaacaacgccctgcagaatcttgctcggaccattagcgaagccggacaagcgatggcctccaccgagggaaacgtgaccgggatgtttgccggggggtccggaggaggaggctcaggaggaaccagccgcttcatgactgaccctcatgccatgcgggacatggccggacgcttcgaagtgcatgcgcagactgtggaggacgaagcccggcggatgtgggcatcggcccagaacatctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggttcgggaggcggtggatcgggggggtcattcgtgatggcttaccccgagatgttggctgctgcggctgacacccttcagagcatcggagccactaccgtcgcctcgaacgccgcggcagccgcccctaccaccggggtggtgcctccagccgccgacgaagtctccgccctgactgccgcgcacttcgccgcccacgcagccatgtaccagtcggtgtcggcccgggcggcagctatccacgatcagttcgtggccacgttagcatccagcgcgtcaagctatgccgccacggaagtggccaacgcagctgcagcctctggaggaagcgggggcggaggaagtggcggcactatcaactaccaattcggagatgtggacgcgcacggcgccatgatccgcgcccaagcgggatcccttgaagcggagcatcaggccattatctccgacgtcttgaccgcctccgacttttggggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 411 - protein, 1189 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGSGGGGSTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGGGSGGGGSTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGEPSSQSTVPIVGIVAGLAVLAVVVIGAVVAAVMCRRKSSGGKGGSYSQAACSDSAQGSDVSLTA

SEQ ID NO: 412 - RNA, 3760 nt, synthetic construct
  source 1..3760: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcgccgaaatgaagactgacgcagccactctggcccaggaggctgggaacttcgaacgcatctcaggagatctcaagacccagatcgatcaggtggaatccactgccggcagcctccaaggccagtggcgaggagccgccggtactgccgctcaggccgcagtggtgcgcttccaggaggccgccaacaagcagaagcaggaactggacgagattagcaccaatatccgccaagccggagtgcaatatagccgggccgacgaggaacagcaacaagccctctcctcccaaatggggttcggcggaggaggaagcggaggcggcggcagcaccgagcagcagtggaacttcgccggaatcgaggccgcagcgagcgcaatccagggcaatgtcacctccattcactccctgctggatgagggcaaacagtccctgactaagcttgccgcagcgtggggaggttccggctccgaagcctaccagggcgtgcagcagaaatgggacgctactgctaccgaactgaacaacgccctgcagaatcttgctcggaccattagcgaagccggacaagcgatggcctccaccgagggaaacgtgaccgggatgtttgccggaggatcgggcgggggcggctccggagggtcctttgtcatggcatacccagagatgctggccgccgccgcggataccctgcagtcgattggagccactaccgtggcctcaaacgccgcagcggctgcccccaccactggagtggtgcccccagcagccgacgaagtctccgccttaactgccgcccactttgccgcccatgcggcgatgtaccagtccgtgtcagcccgcgccgccgcaatccacgatcagttcgtcgcaacactggcgtccagcgcgtcgtcgtacgccgccaccgaggtggccaacgcggcggcggcctcaggggggtccggaggaggaggctcaggaggaaccagccgcttcatgactgaccctcatgccatgcgggacatggccggacgcttcgaagtgcatgcgcagactgtggaggacgaagcccggcggatgtgggcatcggcccagaacatctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggtggaggaagcgggggcggaggaagtactatcaactaccaattcggagatgtggacgcgcacggcgccatgatccgcgcccaagcgggatcccttgaagcggagcatcaggccattatctccgacgtcttgaccgcctccgacttttggggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgggggatccggaggggggggttccggcggcgagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 413 - protein, 1252 aa, synthetic construct
MAVMAPRTLLLLLLGALALTQTWAGSTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAGGSGGGGSGGTSRFMTDPHAMRDMAGRFEVHAQTVEDEARRMWASAQNISGAGWSGGGSGGGGSGGTEQQWNFAGIEAAASAIQGNVTSIHSLLDEGKQSLTKLAAAWGGSGSGGSGGGGSGGTINYQFGDVDAHGAMIRAQAGSLEAEHQAIISDVLTASDFWGGAGSGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGGQWRGAAGTAAQAAVVRFQEAANKQKQELDEISTNIRQAGVQYSRADEEQQQALSSQMGFGGSGGGGSGGSGAGWSGMAEATSLDTMTQMNQAFRNIVNMLHGVRDGLVRDANNYEQQEQASQQILSSGGSGGGGSGGGGSGSEAYQGVQQKWDATATELNNALQNLARTISEAGQAMASTEGNVTGMFAGGSGGGGSGGGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGEPSSQSTVPIVGIVAGLAVLAVVVIGAVVAAVMCRRKSSGGKGGSYSQAACSDSAQGSDVSLTA

SEQ ID NO: 414 - RNA, 3949 nt, synthetic construct
  source 1..3949: mol_type = other RNA
acatttgcttctgacacaactgtgttcactagcaacctcaaacagccaccatggccgtgatggcccccagaactctcttgctgcttctgttgggagctctggcgctgacccagacctgggctggatcgaccgctgcctccgacaacttccaactgtcacaaggaggacaagggtttgccatcccgatcggccaagccatggccatcgccggacaaattcggagcgggggaggaagccccactgtgcacattggtccgaccgccttcctcgggctgggcgtggtggacaacaacggcaacggcgccagggtgcagagggtcgtgggatccgcaccggccgcctctctcggaatttcgaccggcgacgtgattaccgccgtggacggggcgccgattaactccgccacggcaatggccgacgcgctcaacggtcaccacccgggagatgtcatctccgtgacctggcagaccaagagcggaggcactcgcaccggaaacgtcactctcgccgagggcccacccgccggaggttctggaggaggcggctccggtggcgcggagatgaaaaccgatgcggccaccctggcacaggaggctggaaacttcgaaagaatcagcggcgacctcaagactcagatcgaccaggtcgaatcgactgccggaagcctccagggccagtggaggggagccggtggatccggaggagggggatccggcggaaccagccgcttcatgaccgatcctcacgctatgcgcgacatggcgggtcgcttcgaagtgcatgcccagactgtcgaggacgaggcccggcgaatgtgggccagcgcgcagaacatcagcggcgccggttggtcaggaggcgggtccggggggggaggttctggcggtaccgagcagcaatggaacttcgcgggtattgaagccgcggcatccgcgatccagggcaacgtgacctccattcacagcctgttagacgaaggaaagcagagcctgaccaagttggccgccgcctggggggggtcagggtccggaggttccggaggcggtggctcgggcggcaccatcaattaccagttcggggacgtggacgcacacggagccatgatccgggcccaggcgggctccctggaagcggagcatcaggccatcattagcgatgtgctcacggctagcgatttctgggggggagccggaagcggtggcagcgggggtggcggctcagggggagtggatttcggggctctgccacctgagatcaactctgcccggatgtacgcggggccgggcagcgcatcccttgtggccgccgcccagatgtgggatagcgtggcttccgacctcttctccgccgcatccgcctttcaatccgtggtctgggggctgactgtgggctcgtggatcggatcatctgctggcctcatggtggccgcggcatccccatacgtggcctggatgagtgtgaccgctggccaggctgaattgaccgccgcacaagtccgcgtcgctgccgccgcatacgaaaccgcctacgggctgaccgttccgccccccgtgattgccgaaaatagggccgaactcatgatcctcatcgcgactaatctgctgggtcagaacaccccggctatcgccgtgaatgaggccgaatacggcgaaatgtgggcccaggacgccgctgccatgttcggctacgccgccgccactgcgaccgcgaccgccactctgctgcctttcgaagaggcccctgagatgacctccgcgggtggcctgctagaacaagcggcagctgtggaagaagcctcggataccgccgccgctaaccagctgatgaacaacgtgccccaggcgctgcagcagctcgcccagccgactcagggaaccaccccgagctcaaagcttggtggcctgtggaaaaccgtatcgcctcatcgctcaccaatcagcaacatggtcagcatggcgaacaaccacatgggtggctcaggcggcgggggctccggaggcagctttgtgatggcctaccccgaaatgctcgcagccgcagcggataccttgcagtcaattggcgccaccaccgtggcctccaacgccgctgccgccgccccgactacgggggtggtcccgcccgccgcagatgaagtgtccgctcttaccgcagcgcactttgccgcccatgctgccatgtaccagtccgtgtcggctagagctgccgcgatccacgaccagttcgtggctaccctggccagctcggcatcgagctacgccgccaccgaggtggctaacgccgccgccgccagcggggggtccggaggaggaggctcaggaggaggccaatggcggggtgccgccggaaccgccgcccaagcggctgtcgtgcgcttccaggaggccgccaacaagcagaagcaggaactcgacgaaatatcaaccaacatccggcaagctggggtgcagtacagcagagccgatgaggaacagcaacaggcgctctcctcgcaaatgggcttcggaggctcaggagggggcggatcgggtggctccggagcgggatggtccggaatggccgaggccacctccctcgacaccatgacgcagatgaaccaggcctttcgcaatatcgtgaacatgctgcacggcgtgcgcgacggcctcgtgcgggacgccaacaactacgagcaacaggagcaggcgtcacagcagattctgtcctcgggcggttcgggaggcggtggatcgggggggggaggatccggatcagaagcctatcaaggtgtgcagcagaagtgggacgccaccgcgaccgaacttaataacgccttgcagaacttggcccggacgatatccgaagccggccaagccatggcctccacggaaggcaatgtgactggcatgttcgcgggaggaagcgggggcggaggaagtggcggcggtggagccgggagcgccgcgtgccagggattcatcacccaactggggcggaatttccaggtcatctacgaacaggctaacgcccacggtcaaaaggttcaggctgccggcaacaacatggcccagaccgactccgccgtggggtcctcctgggccggaggctccggcggtggcggatcaggaggccctgtgggaggacagtcctccttctattccgattggtactcccccgcctgcggaaaggccggatgtcagacgtataagtgggagacattcctgacctccgagctgccccagtggctctcggccaaccgggctgtgaagccgactggatccgccgcaattgggctgtccatggcgggctcgtcggcgatgatcctggccgcttaccacccacaacagttcatctacgccggatccttgtcggcactgctggacccgagccaggggatgggcggtggttccgggggcgggggatccggaggtttcgccgcgaccgtgccacctccggtcgtggcagccaaccgcgccgaactggccgtcctggcggctaccaacgggggatcgggtggaggggggtcgggaggcagagccgccatggtcgaccctgtggtggtcgccgccaatagatcagccttcgtgcaactcgtgctgtcgaacgggggctccggcggtggtggctcgggcggggagccctccagccaatccaccgtgccgatcgtcggaattgtggccggcctggctgtgctggcagtggtggtcattggagccgtggtggctgccgtgatgtgcagaagaaagagcagcggaggaaagggaggatcctactcacaagccgcgtgctccgatagcgcccagggttcggatgtgtcactcaccgcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 415 - protein, 1162 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 416 - RNA, 3679 nt, synthetic construct
  source 1..3679: mol_type = other RNA
acatttgcttctgacacaactgtgttcactagcaacctcaaacagccaccatggccgcccctggcagcgccagaagacctctgctgctgctgctgctgctgctgctgctgggcctgatgcactgcgcctcagccaccgccgcttctgacaacttccagctgagccagggcggccagggattcgccatccctattggtcaggccatggccatcgccggccagatcagaagtggaggtggctctccaaccgtgcacatcggccccactgccttcctgggcctaggagtggtggataacaacggcaatggcgccagagtgcaaagagtggtgggttcagctcctgccgccagcctggggatcagcaccggcgacgtgatcaccgcagtggacggcgctcctatcaactccgccacagccatggccgatgccctgaacggccaccaccctggagatgtcatctctgtgacctggcagacaaagagcggcggcaccagaacaggaaacgtgaccctggccgaggggcctcctgccggcggctccggaggcggtggatctggaggcgccgagatgaagaccgacgccgctacactggcgcaggaggccggcaacttcgagagaatctcaggggatctgaagacccagatcgaccaggtggaaagcaccgcaggatctcttcagggccagtggcggggcgccgctggcacagccgcccaggctgctgtggtgcggttccaggaggccgccaacaagcagaaacaggagctggatgagatcagcaccaacattcgccaggccggcgtgcaatacagcagagccgacgaagaacagcagcaggctctgtctagccagatgggatttggcggttctggtggtggcggatcaggtggcgtcgacttcggcgccctcccccccgaaatcaacagcgccagaatgtacgccggacctggcagcgccagcctggtggcagcagcccaaatgtgggatagcgtggcttctgacctgttctccgccgctagcgcttttcagtccgtggtgtggggcctgaccgtgggcagctggatcggcagcagcgctggactgatggtggcagctgccagcccctacgtggcctggatgagcgtgacagctggacaggctgaactgacagccgcccaagtgcgggtggctgccgccgcctatgagacagcttacggcctgacagtgccccccccagtgattgccgaaaaccgggccgagctgatgatcctgatcgccaccaatctgctgggccagaacacccctgccatcgccgtgaacgaggcggaatacggcgagatgtgggcccaggacgccgccgccatgttcggctacgcagccgctaccgccaccgccacagccaccctgctgcctttcgaggaagctccagagatgaccagcgctggcggcctattggagcaggccgccgcagttgaggaagcttctgatacagccgccgcgaaccagctgatgaacaacgtgccacaggccctgcagcagctggctcaacctacccagggcaccacacctagctccaagctgggcggtctgtggaagaccgtgagccctcatagatctcccatcagcaatatggtctctatggccaacaaccacatgggcggctccggtggaggcgggagcggcggcacagagcagcaatggaacttcgccggcatcgaggcagccgcttctgccatccagggcaatgtgaccagcatccattctctgttggacgagggcaaacagagcctgaccaagctggccgccgcctggggaggcagcggctctgaggcctaccagggcgttcaacaaaaatgggacgccactgccaccgaactgaacaacgccctgcagaatttagcccggacaatcagcgaggccggccaggctatggcctctactgaaggcaacgtgaccggcatgttcgccggcggcagcggaggcggcggtagtggaggcaccagcagattcatgacggatccccacgccatgcgggatatggccggcagattcgaggtgcatgctcagaccgtcgaggacgaggccaggcggatgtgggctagcgcccagaacatcagcggcgccggctggagcggaatggccgaagctacaagcctggacaccatgacgcaaatgaaccaggcctttagaaacatcgtgaatatgctgcacggcgtgcgcgacgggctggtgcgggacgctaacaactacgagcagcaggagcaggccagccagcagatcctgtcttctggcggcagcggtggaggaggctctggcggctcatttgtaatggcgtatcctgaaatgctggcagccgccgcggataccctccagagcatcggcgccaccaccgtggccagcaacgccgccgctgccgcccccaccacaggtgtggtgccacctgctgccgacgaggtgagcgccctgaccgccgcccacttcgccgctcacgccgcgatgtaccagagcgtgtccgctagagccgctgcaatccacgaccagttcgtggctaccctggccagcagcgcctcgagctacgccgcaactgaggtcgccaacgccgctgctgccagcggcggaagcggcggtggtggaagcggcggaacaatcaactaccagtttggcgacgtggacgcccacggagctatgatcagagcccaggccggcagcctggaagccgaacaccaggccatcatttcagacgtgctgacagcttctgatttctggggtggggccggctctgctgcctgccagggctttatcacacagctgggcagaaatttccaagtcatctacgagcaggcgaatgcccacggccagaaggtgcaggccgcggggaacaacatggcccagacagactcggctgtcggatcttcttgggccgggggcagcggtggcggcggatctggcgggcctgtgggcggacagtctagcttctacagtgactggtactcccctgcatgcggcaaggctggctgtcagacctacaagtgggaaaccttcctgaccagcgagctccctcagtggctgagcgccaatagagcagttaagcctaccggctctgccgccatcggcctgagcatggccgggagtagcgccatgatcctggccgcctaccacccccagcagttcatctatgccggaagcctgagcgccctgctggaccctagccaggggatgggaggaggttcaggtggaggtggatctggcggcttcgccgcgaccgtgcctccacctgtggtcgccgccaatagagctgaactggccgtgctggccgcgacaaacgggggttctggaggtggcgggtctggcggcagagccgctatggtggaccccgtggtggtggccgccaaccggagcgccttcgtgcagctggtgctgagcaacggcggcagcggcggtggcggatcaggcggcctgatccccatcgccgttggcggtgccctggctggcctggtcctgatcgtgctgatcgcctacctggtggggagaaagcggagccacgccggttaccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 417 - protein, 1162 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGAEMKTDAATLAQEAGNFERISGDLKTQIDQVESTAGSLQGQWRGAAGTAAQAAVVRFQEAANKQKQELDEISTNIRQAGVQYSRADEEQQQALSSQMGFGGGGSGGGGSTEQQWNFAGIEAAASAIQGNVTSIHSLLDEGKQSLTKLAAAWGGSGSEAYQGVQQKWDATATELNNALQNLARTISEAGQAMASTEGNVTGMFAGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGTSRFMTDPHAMRDMAGRFEVHAQTVEDEARRMWASAQNISGAGWSGMAEATSLDTMTQMNQAFRNIVNMLHGVRDGLVRDANNYEQQEQASQQILSSGGGGSGGGGSTINYQFGDVDAHGAMIRAQAGSLEAEHQAIISDVLTASDFWGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 418 - RNA, 3679 nt, synthetic construct
  source 1..3679: mol_type = other RNA
acatttgcttctgacacaactgtgttcactagcaacctcaaacagccaccatggccgcccctggcagcgccagaagacccctgttactgttgctgctgttattgctgctgggactgatgcactgcgccagcgctaccgctgcgtctgataacttccagctgagccagggtgggcagggctttgctattcccatcgggcaggccatggccatcgccggccagatccgcagcggaggtggctctccaaccgtgcacatcggccctacagctttcctgggcctgggagtggtggacaacaacggcaacggcgcaagggtgcaaagagtggtgggctcagcccctgctgcctccctgggcatcagcaccggcgacgtgatcaccgccgtggacggcgcccctatcaacagcgccacagccatggccgacgccctcaacggccaccatcctggagatgtgatcagcgtgacctggcagacaaagagcggcggcaccagaaccggcaatgtgaccctggccgaaggccctcctgccggaggctctggaggcggtggatctggcggagttgatttcggcgccctgccccccgaaattaacagcgccagaatgtacgccggccccggctccgctagcctggttgcagccgctcagatgtgggatagcgtggccagcgacctgttcagcgcggcctctgccttccaaagcgtggtgtggggcctgacagtggggagctggatcggctccagcgccggactgatggtggctgccgcaagcccttacgtggcctggatgagcgtcacagccgggcaggccgagctgaccgccgctcaggttcgggtggccgctgccgcctatgaaaccgcctacggattaacagtgccacctcctgtcatcgccgagaaccgggctgagctgatgatcctgatcgccaccaatctgctgggccagaatacccctgctatcgccgtgaacgaggccgaatacggcgagatgtgggcccaggacgcagccgctatgttcggctacgccgccgccaccgccaccgctaccgctaccctgctccccttcgaagaagcccctgagatgacatctgccggcggcctgcttgagcaggccgccgccgtggaggaagcctccgacaccgccgcagctaaccagctgatgaacaacgtgccccaggctctgcagcagctggcccaacctacccagggcaccacccctagcagcaagctgggaggactgtggaagaccgtctcccctcacagaagccctatctctaacatggtctccatggctaacaaccacatgggcggcagcggtggtggcggatcaggcggagccgaaatgaaaaccgacgccgccaccctggcccaagaggcgggcaacttcgagcggatcagcggggacctgaagacccagattgaccaggtggagagcaccgccggctccctgcaaggccaatggagaggcgccgccggtacggctgcccaggccgctgtggttagatttcaggaggctgctaacaagcagaagcaggagctggacgagatcagcaccaacatccggcaggcaggcgtgcagtacagtagagccgacgaggaacagcagcaagctctgagcagccagatgggcttcggtggaggcgggagcggaggcggcggtagtacagagcagcagtggaactttgccggaatcgaggcggcagccagtgccatccagggcaacgtgacatcaattcacagcctgctggacgagggcaaacagagcctgaccaagctggccgcagcctggggaggctctggctctgaagcttaccagggcgttcagcaaaagtgggacgcaacagccaccgaactgaacaacgccctgcagaacctggcccggaccataagcgaggccggacaggcaatggccagcaccgagggcaatgtgaccggaatgttcgccggcggcagcggtggaggaggctctggaggtagttttgtgatggcttacccagaaatgctggccgctgccgccgacaccctgcagtctatcggcgcaacaaccgtggcctctaatgccgccgccgccgcccctaccacaggcgtggtgccgcctgccgctgacgaggtgtcagctctgaccgcggcgcatttcgcggcccacgccgccatgtaccagagcgtgtctgccagagccgccgccatccacgaccagttcgtggccaccctggccagcagcgccagtagctacgctgccacagaagtggctaacgcagccgccgccagcggtggttccggcggtggtggaagcggcggaacctccagattcatgaccgatccccacgccatgagagacatggccggtagattcgaggtgcacgcccagaccgttgaagatgaagcccggagaatgtgggctagcgcccaaaatatctccggcgccggatggtctggcatggctgaggccacaagcttggatacgatgacccagatgaaccaggcctttagaaacatcgtgaacatgctccacggcgttagagacggcctggtgcgggacgccaacaattacgagcagcaggaacaggcctctcagcagatcctgagctctggtggcggcggatctggcggaggaggttcaacaatcaactaccagtttggcgatgtggatgcccacggagccatgatcagagcccaggctggctctcttgaagccgagcaccaggccatcatcagcgatgtgctgactgcaagcgacttctggggcggtgccggctccgctgcttgtcagggcttcatcacacagctgggaagaaacttccaggtgatctacgagcaggccaatgcgcatggccaaaaagtgcaggcagccggaaacaacatggcccagaccgacagcgctgtgggaagcagctgggccggcggcagcggtggaggtggatctggcggtccagtgggcggccagagcagcttctacagcgactggtacagccctgcgtgcggcaaggccggctgccagacctacaaatgggagacattcctgacatctgagctgcctcagtggctgagcgccaatcgggccgtgaagcccaccggcagtgccgccatcggactgagcatggctggctcctccgccatgattctggctgcctaccacccccagcagttcatctatgccgggagcctgagcgccctgctggaccccagccagggaatgggtggcgggtctggcggtggcggatcaggcggcttcgccgccaccgtgcctccacctgtggtcgccgccaacagagccgagctggctgtgctggccgctacaaacgggggcagcggaggcggaggtagtggcggcagagccgccatggtggaccctgtggtggtggccgccaaccggagcgcttttgtgcagctggtgctgagcaatggcggctctggtggtggagggtcaggcgggctgatccctatcgccgtgggcggtgccctggccggcctagtgctgatcgtgctgatcgcctacctggtgggcagaaagcggagccacgccggctaccagacaatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 419 - protein, 1225 aa, synthetic construct
MAAPGSARRPLLLLLLLLLLGLMHCASATAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAGGSGGGGSGGAEMKTDAATLAQEAGNFERISGDLKTQIDQVESTAGSLQGQWRGAGGSGGGGSGGTSRFMTDPHAMRDMAGRFEVHAQTVEDEARRMWASAQNISGAGWSGGGSGGGGSGGTEQQWNFAGIEAAASAIQGNVTSIHSLLDEGKQSLTKLAAAWGGSGSGGSGGGGSGGTINYQFGDVDAHGAMIRAQAGSLEAEHQAIISDVLTASDFWGGAGSGGSGGGGSGGVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMGGSGGGGSGGSFVMAYPEMLAAAADTLQSIGATTVASNAAAAAPTTGVVPPAADEVSALTAAHFAAHAAMYQSVSARAAAIHDQFVATLASSASSYAATEVANAAAASGGSGGGGSGGGQWRGAAGTAAQAAVVRFQEAANKQKQELDEISTNIRQAGVQYSRADEEQQQALSSQMGFGGSGGGGSGGSGAGWSGMAEATSLDTMTQMNQAFRNIVNMLHGVRDGLVRDANNYEQQEQASQQILSSGGSGGGGSGGGGSGSEAYQGVQQKWDATATELNNALQNLARTISEAGQAMASTEGNVTGMFAGGSGGGGSGGGGAGSAACQGFITQLGRNFQVIYEQANAHGQKVQAAGNNMAQTDSAVGSSWAGGSGGGGSGGPVGGQSSFYSDWYSPACGKAGCQTYKWETFLTSELPQWLSANRAVKPTGSAAIGLSMAGSSAMILAAYHPQQFIYAGSLSALLDPSQGMGGGSGGGGSGGFAATVPPPVVAANRAELAVLAATNGGSGGGGSGGRAAMVDPVVVAANRSAFVQLVLSNGGSGGGGSGGLIPIAVGGALAGLVLIVLIAYLVGRKRSHAGYQTI

SEQ ID NO: 420 - RNA, 3868 nt, synthetic construct
  source 1..3868: mol_type = other RNA
acatttgcttctgacacaactgtgttcactagcaacctcaaacagccaccatggccgcacctggctcagcccgcagacctctgctgctgctgctattgctgctgctcctgggactgatgcactgcgcctctgccaccgccgcttccgacaacttccagctgagccagggcggccaaggcttcgccatccccatcggccaggctatggcaatcgcgggacaaatcagaagcggaggtggctctcccactgtgcacatcggccctaccgcattcctgggcctgggcgtggtggacaacaacggcaacggcgcccgggtgcagagagtggtgggctcagctcccgccgccagcctgggcataagcaccggggatgtgataacagccgtggatggcgcccctatcaacagcgctactgccatggccgatgccctgaacggccaccaccccggcgacgtgatcagcgtgacctggcagactaagagcggcggcacaagaacaggcaatgtgaccctggccgaaggccctcctgccggcggctctggaggcggtggatctggcggcgccgaaatgaagacagacgccgccaccctggcccaggaggccggaaatttcgaaagaatcagcggcgatctgaagacccagatcgaccaggtcgagagcacagcgggaagcctgcagggccagtggagaggcgccggcggctccggtggtggcggatcaggcggcacaagccgattcatgaccgatccgcacgccatgcgggacatggccgggaggttcgaggtgcacgcccagaccgtggaagatgaagcccggcggatgtgggctagcgctcagaacatcagcggggccggatggagcggaggcgggagcggaggcggcggtagtggaggcaccgagcagcaatggaacttcgctggaatcgaggccgccgccagcgctattcagggcaacgtgaccagcatccactctctgctggacgagggcaaacagtccctgaccaaactggccgccgcctggggcggcagcggctctggcggaagcggtggaggaggctctggcggaaccataaactaccagttcggcgacgtggatgctcacggcgccatgatccgggcccaggccggaagcctggaagccgaacaccaggccatcatcagcgacgtgctgaccgcatctgacttctggggcggcgcgggctccggaggttccggcggtggtggaagcggaggcgtggacttcggcgctctcccccctgagatcaacagcgccagaatgtacgccggaccaggctcagcctccctggtagcggcggctcagatgtgggacagcgtggctagcgacctgttcagcgccgccagcgccttccagagcgtggtctggggactgaccgtgggcagctggatcggaagcagcgccggcctgatggtggccgccgccagcccctacgtggcctggatgagcgttaccgccggtcaagccgagctgacagccgctcaggtgcgcgtggccgccgctgcctacgagacagcctatggcctgaccgtgcccccccccgtgattgctgaaaaccgggccgagctgatgatcctcatcgccaccaacctgcttggccagaacacccctgccatcgccgtgaacgaggccgaatacggcgagatgtgggctcaggacgccgccgccatgttcggctacgccgccgccacagctaccgccaccgctacactattaccattcgaagaggcccctgaaatgacctccgccggcggcctgctggaacaagccgcggccgtggaagaggccagcgataccgctgccgccaaccagctgatgaacaatgtgccacaggccctgcagcagctggctcaacctacccagggcaccacacctagcagcaagctgggaggcctgtggaagaccgtgtccccacacagaagccccatcagcaacatggtctccatggctaacaaccacatgggcggaagcggtggcggcggatctggcggcagttttgtgatggcttatcctgagatgctggccgctgctgccgacaccctgcagagtatcggtgccaccaccgtggccagcaacgcagccgcggctgcccctacaaccggcgtggtgcctcccgctgccgacgaggtgtctgccctgacggccgcccatttcgccgctcatgccgctatgtaccagagcgtgtccgccagagccgccgccattcatgaccagttcgtagcgacactggcctcttctgcctcctcatatgccgccacagaggtggccaacgccgctgccgcatccgggggcagcggcggaggaggttcaggcggcggacagtggcggggcgctgctggcaccgccgcccaggcggcagtggtgagatttcaggaggctgccaacaagcagaaacaggagctggacgagatctccaccaatatcagacaggccggcgttcagtacagccgggctgacgaggaacagcagcaggccctgtcaagccagatgggatttggcggtagcggtggaggtggatctggcggcagcggagccggatggtccggcatggccgaggccacctctctggatacaatgacccaaatgaaccaagcattccggaacatcgtgaacatgctgcacggcgttagagacggccttgtgcgggacgccaacaactacgagcagcaggagcaggcaagccagcagatcctatctagcggtggcagcggaggtggcgggtctggcggtggcggatcaggctctgaagcctaccagggagtccagcagaaatgggacgccacagctacggaactgaacaatgccctgcaaaatctggctagaaccatcagcgaggcgggacaggctatggccagcaccgaaggcaatgtcacaggcatgttcgccggcggctctggaggcggaggtagtgggggcggcggcgctggctcggccgcttgccaggggttcatcacccagctgggcagaaacttccaggtgatctacgagcaggccaacgctcacggccagaaggtgcaggcggccggaaacaacatggcccagaccgacagcgccgtggggtcaagctgggccggcggaagcggtggtggagggtcaggcggccctgtgggcggacagtctagcttctacagcgactggtacagccctgcctgcgggaaggccggttgtcagacatacaagtgggagacattcctgaccagcgagctgcctcagtggctgtctgcgaatagagccgtgaagcctaccgggagcgcagccatcggcctgagtatggccggctcgtctgccatgatcctggccgcttaccaccctcagcaatttatctacgcaggatctctgagcgccctgctggacccttctcaaggcatgggcggtggaagcggaggaggcggttctggaggctttgccgccaccgtgccacctcctgtggttgccgctaaccgggccgaactggctgtgctggctgcgacgaacggcggatctggcggcggcggtagcggaggcagagccgcaatggtggaccccgtggtcgtggccgcaaatagaagcgcctttgtgcagctggtgctgtctaatggcgggagcggcgggggcggtagcggcggcctgatccctattgccgtcggcggcgcactggccggcctggtgctgatcgtgctgatcgcctacctggttggcagaaagagaagtcacgccggttatcaaacaatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 421 - protein, 1185 aa, synthetic construct
MAVMAPRTLLLLLLGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGEPSSQSTVPIVGIVAGLAVLAVVVIGAVVAAVMCRRKSSGGKGGSYSQAACSDSAQGSDVSLTA

SEQ ID NO: 422 - RNA, 3748 nt, synthetic construct
  source 1..3748: mol_type = other RNA
acatttgcttctgacacaactgtgttcactagcaacctcaaacagccaccatggctgtgatggccccgcgcaccctcctgctgctcctgctgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 423 - protein, 1158 aa, synthetic construct
MAAPGSARRPLLLLLLLLLLGLMHCASASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGLIPIAVGGALAGLVLIVLIAYLVGRKRSHAGYQTI

SEQ ID NO: 424 - RNA, 3667 nt, synthetic construct
  source 1..3667: mol_type = other RNA
acatttgcttctgacacaactgtgttcactagcaacctcaaacagccaccatggccgctcctggcagcgctcggcggccactgctgctgctactcctcctgctgctgctgggactgatgcactgcgcgagcgcctccagagcatttatcatcgatcctaccatttctgccatcgacgggctgtacgatctgctgggcatcggcatccctaaccagggcggcatcctgtatagctctctggagtactttgagaaggctctggaggaactggctgccgcgtttcctggcgatggatggctgggcagcgccgccgacaaatacgccggaaaaaacagaaaccacgttaacttcttccaggagctggccgacctcgacagacagctcatctccctgatacatgatcaggccaacgccgtccagaccacccgggacatcctggaaggcgccaaaaagggcctggagttcgtgcggcccgtggccgtggatctgacttacatccccgtggtggggcacgctctgagcgccgccttccaagcccctttctgcgccggtgctatggccgtcgtaggcggcgccctggcttatctggtggttaagaccctgatcaacgccacacagctgctgaagttacttgccaagctggccgagctggtcgctgctgccatcgctgatataatcagcgatgtggcggacatcatcaagggcaccctgggcgaggtgtgggagttcatcaccaacgccctgaacggcctgaaggagctgtgggataagctgacagggtgggtcacaggacttttcagcagaggctggtccaatctggaaagcttcttcgctggggtgcctggcctaaccggcgccacaagcggcctgagtcaggtgacaggactgttcggagccgctggcctgtctgcttctagcggcctggctcacgccgacagtctggcctctagcgccagcctgcctgccctggccggcatcggaggtggctctggcttcggcggcctgccaagcctggcacaggtgcacgccgccagcacacggcaagccctcagacctagagccgatggccctgtgggcgccgccgccgaacaggtcggaggccagagccagctggtgtccgcccaaggtagccagggcatgggaggacctgtaggcatgggcggcatgcatcctagcagcggcgcctcaaagggcaccaccacaaagaagtacagcgagggcgcagccgccggaactgaggacgcagaacgggcccctgtcgaagccgacgccggcggcggccaaaaagtgctggttcggaacgtggtggggggatctggaggcggtggatctggcggcatcaccgacagactgcctgccaaggcccctctgctggaactggacggcggcagcggtggtggcggatcaggaggcgctatcacaatcaacatggtcaatcccaccggttttatcagatacgaggacgtggaacaggaggccatgacaagcgacgtgaccgtgggccccgctccaggacagtaccagctgagccacctgcggctgcttgaggccgaagccatccacgtgattagagaagtggccgccgaatttgagagacccgtgcttctgttctccggaggcaaggacagcatcgtgatgctgcacctggcattgaaagcctttcggcctggcagactgccttttcctgtgatgcacgtggacaccggccacaacttcgacgaggtgatcgccaccagagacgaactggtggcggcagccggagtgaggctggtggtggccagcgtgcaggatgatatcgatgccggcagagtggtggaaaccattcccagcagaaaccccatccaaacagtgaccttactgcgggccatcagagagaaccagttcgacgcggccttcggcggcgcccggagagatgaggaaaaggcccgggccaaagagagagtgttttctttcagagacgagttcggccagtgggatcccaaggcccagcggcctgagctgtggaacctgtacaacggaagacaccacaagggagaacacatccgggtgttccctctgagcaactggaccgagttcgatatttggagctacataggcgccgagcaggtgcgtctgcccagcatctacttcgcccatagaagaaaggtgttccaaagagatggcatgctgctggccgtgcaccggcacatgcagcctagagccgatgaacctgtgttcgaggccaccgtgcgcttccggacagtgggcgacgtcacctgcaccggctgtgtggagtcttctgcctctaccgtcgcagaagtgatcgccgagacagccgtggccagactgaccgagagaggcgctacccgggccgacgatcggatctctgaggccggcatggaagatagaaagagacagggctacttcggaggatccggtggaggcgggagcggcggcaagcgggtcgagatcgaggtggacgatgacctgatccagaaggtgattagaggaggcagcggaggcggcggtagtggcggggctccacctgccctgtcccaggaccgcttcgccgacttcccagccctccctctggaccctagcgccatggtggcccaggtgggccctcaggtggtgaacatcaataccaagctgggctacaacaacgctgtgggggccggcacaggcatcgtgatcgaccccaatggagtggtgctgacaaacaatcacgtgatcgccggcgccaccgatatcaacgccttcagcgtgggcagcggccagacatacggcgtggacgtggttggatacgacagaacacaggacgtggccgtgctgcagctgagaggcgcgggcggactaccaagcgccgccatcggcggaggtgtggccgtgggcgagcctgtggtggccatgggcaacagcggcggtcagggcggcacacctcgggctgtgcccggcagagtggtggctttgggccagaccgtgcaggctagcgacagcctgaccggggccgaggaaaccctgaacggcctgatccagttcgacgctgctatccagcccggcgacagcggggggcccgtggtgaacggccttgggcaggttgtgggcatgaacggcggaagcggtggaggaggctctggcggcgccctggcctggatgtgcgacagagctgttgatctgctgctgctgggcggcagcggcggtggtggaagcggtggtgcgatccacgtgatgaccacagtgttggccacactgccggcagacggcggttccggtggcggcggatctggcggcaccaccagggtgatggccacgcgcagaaatgtgctggaccgtcaaggcggctctggcggaggaggttcaggcggcgtggcccgggtcctgagacctggcggacacttcctgtacaccgatggcggatctggtggaggtggatctggcggcctgattcctatcgccgtggggggcgccctagcaggcctggtcctgatcgtgctgatcgcctacctggtgggcagaaagcgcagccacgccggctatcagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 425 - protein, 1185 aa, synthetic construct
MAVMAPRTLLLLLSGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGEPSSQSTVPIVGIVAGLAVLAVVVIGAVVAAVMCRRKSSGGKGGSYSQAACSDSAQGSDVSLTA

SEQ ID NO: 426 - RNA, 3748 nt, synthetic construct
  source 1..3748: mol_type = other RNA
acatttgcttctgacacaactgtgttcactagcaacctcaaacagccaccatggctgtgatggccccgcgcaccctcctgctgctcctgtcgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 427 - protein, 1195 aa, synthetic construct
MAVMAPRTLLLLLLGALALTQTWAGSSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGITDRLPAKAPLLELDGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGKRVEIEVDDDLIQKVIRGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGALAWMCDRAVDLLLLGGSGGGGSGGAIHVMTTVLATLPADGGSGGGGSGGTTRVMATRRNVLDRQGGSGGGGSGGVARVLRPGGHFLYTDGGSGGGGSGGENLRMKLPKPEPSSQSTVPIVGIVAGLAVLAVVVIGAVVAAVMCRRKSSGGKGGSYSQAACSDSAQGSDVSLTA

SEQ ID NO: 428 - RNA, 3778 nt, synthetic construct
  source 1..3778: mol_type = other RNA
acatttgcttctgacacaactgtgttcactagcaacctcaaacagccaccatggctgtgatggccccgcgcaccctcctgctgctcctgctgggagcgctcgcattgacccagacttgggccgggtcatcccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggaggttctggtggcgggggctccggtggaatcaccgaccggctcccggccaaggctcccctgctggagcttgatggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggaggctcagggggtggaggctcgggcggaaagcgcgtcgagatcgaagtcgacgatgatctgatccaaaaggtcatccggggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccgggggcgccctggcctggatgtgcgacagagctgtggatttgctccttctggggggatcgggtggtggcggatctggaggggccatccacgtgatgacgactgtcctggccaccctgccggccgatggcggctccggcggaggaggttcgggcggaactactagagtgatggccacacgacggaacgtgctggaccgccaggggggatccggcggcgggggcagcggaggagtggcccgcgtccttagacctggaggacacttcctgtataccgacggaggatccggaggaggaggatcaggcggagagaacttgagaatgaagctgcccaagcccgaaccgagttcccagtcaaccgtgcccatcgtcgggatcgtggcaggactcgccgtgttggcagtcgtcgtcattggcgccgtggtggccgccgtgatgtgccgccggaagtcctcaggggggaagggaggctcgtacagccaggccgcctgttccgactcggcccaggggtccgacgtgtcccttactgcatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 429 - protein, 1006 aa, synthetic construct
MAAPGSARRPLLLLLLLLLLGLMHCASASRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVVGGSGGGGSGGAITINMVNPTGFIRYEDVEQEAMTSDVTVGPAPGQYQLSHLRLLEAEAIHVIREVAAEFERPVLLFSGGKDSIVMLHLALKAFRPGRLPFPVMHVDTGHNFDEVIATRDELVAAAGVRLVVASVQDDIDAGRVVETIPSRNPIQTVTLLRAIRENQFDAAFGGARRDEEKARAKERVFSFRDEFGQWDPKAQRPELWNLYNGRHHKGEHIRVFPLSNWTEFDIWSYIGAEQVRLPSIYFAHRRKVFQRDGMLLAVHRHMQPRADEPVFEATVRFRTVGDVTCTGCVESSASTVAEVIAETAVARLTERGATRADDRISEAGMEDRKRQGYFGGSGGGGSGGAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNGGSGGGGSGGLIPIAVGGALAGLVLIVLIAYLVGRKRSHAGYQTI

SEQ ID NO: 430 - RNA, 3211 nt, synthetic construct
  source 1..3211: mol_type = other RNA
acatttgcttctgacacaactgtgttcactagcaacctcaaacagccaccatggccgcccctggatcagccaggcgcccccttctgctgctgctcctcctgctgcttctgggtctgatgcattgcgccagcgccagccgcgccttcattattgaccctaccatttccgccattgacggtctgtacgatctgctgggaatcggcatcccaaaccaaggagggatcctctactcctcgttggaatacttcgagaaggcactcgaggaacttgccgccgcattcccgggcgatggatggctgggaagcgctgctgacaaatacgcggggaagaaccggaaccatgtcaacttcttccaagagctcgccgacctggaccggcagctcatatccctgattcacgaccaggccaatgctgtgcagaccactcgggatatcctggagggagctaagaagggattggaattcgtgcggcctgtggctgtggacctgacctacattccggtggtcggccacgcgctgtccgcggcgttccaggcgcctttctgtgccggagctatggccgtggtcggtggagctctcgcctacctcgtggtcaaaaccctgatcaatgccacccagctgctgaagctgctggctaagctcgcggagttggtcgccgccgcgattgccgatatcattagcgacgtggcagacatcatcaagggcaccctcggagaagtctgggaattcatcaccaacgccctgaacggtctgaaggaactgtgggataagctgactggatgggtcaccggactgttcagccgcggatggtccaacctggagagcttcttcgccggagtgcccggactcaccggtgccacctccgggctttcccaagttaccggactcttcggggccgccggcctgtcggcctcatccgggctggcacacgccgatagcctggcatcctcggcatcgctccccgcgctcgcgggcatcgggggtggcagcggatttggcggactgccctcgctggcacaagtgcacgccgccagcactcggcaggcgctgcgcccgagagccgacggccccgtgggagccgctgcggaacaagtcggtggccagtctcaacttgtgtccgcgcagggaagccaggggatgggtggccctgtgggaatgggcggaatgcatccaagctccggggcctcgaagggcactaccaccaagaagtactccgagggcgctgcggccggcactgaagatgctgaacgggccccggtggaggccgatgccggtggcgggcaaaaggtgctcgtacggaatgtcgtgggtggatccgggggaggcggttccggcggtgccatcaccatcaacatggtcaaccccactggctttatccgctatgaggatgtggaacaggaggccatgacctcagacgtgaccgtgggacccgcccccggccagtaccagctgtcccacctccgcctgctcgaggccgaagcgattcacgtgatcagggaagtggcggccgaatttgagcgcccggtgctgctgttcagcggtggaaaggatagcattgtgatgttgcatttggccctcaaggccttccgacctggtcggctcccgttccctgtgatgcatgtggataccggacacaacttcgacgaggtcatcgcaacgcgcgacgagttggtggcagccgccggcgtgcgcctggtagtggccagcgtgcaagacgacatcgacgccggcagagtggtggaaaccatcccctcccgcaaccctattcagaccgtgaccctgctgagagccattagggaaaaccagttcgacgctgccttcggaggcgcccggcgggatgaggagaaagcgcgcgcgaaggaaagagtgttctcctttcgggacgagtttggacagtgggaccctaaggcgcaacggcccgagttgtggaacctgtacaacggacggcaccataagggggagcacatccgggtctttcctctgtccaactggacagagttcgatatttggagctacattggagccgaacaagtccggctgccgtccatctacttcgcacaccggagaaaagtgttccagcgcgacggcatgctcctcgcggtgcacaggcatatgcagcccagggcagacgagccggtgtttgaagccaccgtcagattccgcacagtgggcgacgtgacttgcaccggctgcgtggaatcctccgcgtccaccgtggccgaagtgatcgctgagactgcggtcgccagattgaccgagcggggtgccacaagagccgatgaccgcattagcgaagcggggatggaggatcggaagagacaaggatactttggtggaagcggcggagggggaagcgggggcgcgccgcccgcgttaagtcaggaccggttcgccgacttcccggccctgcccctggacccttcggcgatggtggcccaagttggcccccaagtggtgaacattaacactaagctgggatacaacaacgctgtgggcgcgggcactggaatcgtcatcgaccctaacggcgtggtgctaaccaacaaccatgtgatagccggggccaccgatatcaacgccttcagcgtggggtccggacagacttacggagtggacgtcgtgggttacgacaggacccaggacgtggccgtgctgcaactgaggggagccggaggactgcccagcgccgcgatcggaggaggggtggccgtgggtgaaccggtggtggccatgggaaattccggcggacagggaggcaccccgagggcggtgcccggccgcgtggtcgctctcggtcaaaccgtgcaggcctccgacagcctgactggcgccgaggaaactctgaacggcctgatccagttcgatgcggccatccagccgggagactcggggggacctgtggtcaacggactgggacaggtcgtgggcatgaacggcggctcgggagggggtggctccggtggcctgattccaatcgccgtcgggggagcactggccggactggtgctcattgtgctgatcgcttacctcgtgggaagaaagcgctcccacgccggataccagaccatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 431 - protein, 844 aa, synthetic construct
MAAPGSARRPLLLLLLLLLLGLMHCASADFGALPPEINSARMYAGAGAGPMMAAGAAWNGLAAELGTTAASYESVITRLTTESWMGPASMAMVAAAQPYLAWLTYTAEAAAHAGSQAMASAAAYEAAYAMTVPPEVVAANRALLAALVATNVLGINTPAIMATEALYAEMWAQDALAMYGYAAASGAAGMLQPLSPPSQTTNPGGLAAQSAAVGSAAATAAVNQVSVADLISSLPNAVSGLASPVTSVLDSTGLSGIIADIDALLATPFVANIINSAVNTAAWYVNAAIPTAIFLANALNSGAPVAIAEGAIEAAEGAASAAAAGLADSVTPAGLGASLGEATLVGRLSVPAAWSTAAPATTAGATALEGSGWTVAAEEAGPVTGMMPGMASAAKGTGAYAGPRYGFKPTVMPKQVVVGGSGGGGSGGDFALLPPEVNSARMYTGPGAGSLLAAAGGWDSLAAELATTAEAYGSVLSGLAALHWRGPAAESMAVTAAPYIGWLYTTAEKTQQTAIQARAAALAFEQAYAMTLPPPVVAANRIQLLALIATNFFGQNTAAIAATEAQYAEMWAQDAAAMYGYATASAAAALLTPFSPPRQTTNPAGLTAQAAAVSQATDPLSLLIETVTQALQALTIPSFIPEDFTFLDAIFAGYATVGVTQDVESFVAGTIGAESNLGLLNVGDENPAEVTPGDFGIGELVSATSPGGGVSASGAGGAASVGNTVLASVGRANSIGQLSVPPSWAAPSTRPVSALSPAGLTTLPGTDVAEHGMPGVPGVPVAAGRASGVLPRYGVRLTVMAHPPAAGGSLIPIAVGGALAGLVLIVLIAYLVGRKRSHAGYQTI

SEQ ID NO: 432 - RNA, 2725 nt, synthetic construct
  source 1..2725: mol_type = other RNA
acatttgcttctgacacaactgtgttcactagcaacctcaaacagccaccatggccgcacctggatccgcaagacgccctttactccttctcctgcttctgctgctgctgggcctcatgcattgcgcctccgccgatttcggagccctgccacctgaaatcaactcggccaggatgtacgccggagctggagctggcccgatgatggccgcaggagctgcttggaacggtctggcagcggaactcggcactactgccgcctcctacgagagcgtgatcactcgccttaccaccgaaagctggatgggaccggcgtccatggccatggtcgcggctgctcaaccctacctggcgtggcttacctacaccgctgaagcggcagcccatgctggatcccaagcaatggcgtccgcagctgcatacgaggcagcctacgccatgactgtgccaccggaggtcgtggcagccaatcgagccctccttgccgctttggtggccacgaacgtgctgggcatcaacactcccgcaattatggctaccgaggcgctctacgcggagatgtgggcccaggacgctctggctatgtacggctacgctgccgcatcgggagcagccggcatgctacaacctctgtcacctccaagccaaaccaccaatcccggaggccttgctgcccaatcagctgccgtgggatctgcggcagccactgctgcggtcaatcaagtctccgtggccgatctcatttcctccctgcccaacgccgtgtcgggcttggcatcgcccgtgacttcggtcctggacagcaccggtctgtccggcattatcgccgacatcgatgccctgcttgcgacccccttcgtcgccaatatcatcaactccgccgtgaacaccgccgcttggtacgtgaacgcggccatcccaaccgccattttcctggcgaacgctctcaactcgggagctcctgtcgccatcgccgaaggcgccattgaagcagcggaaggagccgcgtcagcagctgcagccggattggcggatagcgtgactccagccggacttggggcttccttgggggaagccactctggtcggtcggcttagcgtgccagccgcatggtccactgccgcaccagcaaccaccgcgggagctactgccttggaagggtccggttggacggtcgccgcagaggaagccggacccgtgactgggatgatgcccggaatggcttccgcggccaagggaactggagcctatgccgggcctagatatggcttcaagcctaccgtcatgcctaaacaggtcgtcgtgggagggagtgggggtggaggatcgggaggagacttcgcgctgcttccgcctgaagtgaacagcgcccggatgtacaccggacctggagcaggatccctgttggccgctgcaggaggttgggactcgttggctgcggaactggccaccaccgctgaggcctacgggtccgtgctctccggcttggcggcactgcactggaggggaccagctgccgaatccatggccgtgactgccgctccgtacattggctggctgtacaccaccgccgaaaagacccagcagaccgccattcaagcacgggcagcagctcttgcctttgagcaggcgtacgcgatgactctccctccgcctgtggtcgctgccaaccgcatccagctgctggccctgatcgcgaccaacttcttcggccagaacacagccgccatcgctgccaccgaagcacagtacgccgaaatgtgggcgcaggatgctgccgcgatgtacggatacgccaccgcatcagccgctgcggcattgctgacaccgttctcccctccgcgccaaacgaccaacccagcgggtctgactgcacaggcagccgctgtgtcccaagccactgatccgctctccctgctgatcgaaactgtgactcaagccctgcaagccctgaccatcccttccttcatccctgaggacttcaccttcctcgacgccatcttcgctggctacgcgaccgtgggagtgacccaggacgtggaaagctttgtggccggaaccatcggagcagagtccaacctgggactgctgaacgtcggggatgagaaccccgcggaagtgacccccggcgatttcggcattggggagctggtgtccgctacctcacccggtggaggggtgtcggcatccggtgctggaggagctgccagcgtgggcaatactgtgctcgcctcagtcggacgcgccaacagcattggccagctgagcgtgcctccgtcatgggccgcaccctcaactcgccctgtgtcagccctgagccctgctgggctgaccaccctcccgggaacagacgtggctgagcacggcatgccgggagtcccaggagtgccagttgcagccgggagagctagcggggtgctccctagatacggagtccggctcaccgtgatggcccatccaccggcagcaggaggctcgctgatcccgatagcagtgggaggagcgcttgcgggcttggtgttgattgtcctcatcgcctacctcgtgggaagaaagcggtcacacgccggataccagaccatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 433 - protein, 19 aa, Homo sapiens
MGFLPKLLLLASFFPAGQA

SEQ ID NO: 434 - protein, 31 aa, Homo sapiens
LLWVLGLLVGLLLLLLGLGACYTWRRRRVCK

SEQ ID NO: 435-436 - skipped (2 entries)

SEQ ID NO: 437 - protein, 725 aa, Mycobacterium tuberculosis
MHHTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAAS

SEQ ID NO: 438 - protein, 775 aa, synthetic construct
MGFLPKLLLLASFFPAGQAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLLWVLGLLVGLLLLLLGLGACYTWRRRRVCK

SEQ ID NO: 439 - RNA, 2518 nt, synthetic construct
  source 1..2518: mol_type = other RNA
acatttgcttctgacacaactgtgttcactagcaacctcaaacagccaccatgggctttttgccgaagctgttactgctggcctcattctttccggccggtcaagcgatgacggcggccagcgacaactttcagttgtctcagggcggtcaaggctttgccatacccatcggtcaagctatggccattgcgggccagattcgttcgggcggcggcagtccgactgtgcatatcggtccgactgcgtttttaggtctgggtgtggtcgacaacaacggaaatggtgcacgcgtccaacgcgttgtgggctcagcaccagcggcgtcactgggaatcagcacgggtgacgtaattactgcagtggacggtgcgccgattaactctgccacggccatggcggatgctctgaacggtcaccacccgggggatgttatttccgtcacttggcaaaccaaatctggtgggacccgtacgggcaacgttaccctggcggaagggccgccggcagagttcatggttgatttcggtgcccttccgcccgagatcaactcggcccgtatgtatgctggccccggctctgctagtctggtggctgcggcgcaaatgtgggatagtgtagcctctgaccttttcagtgcggctagcgcgttccagtcggtcgtgtggggtcttactgttggctcgtggattggctcatcagcgggtctgatggtggcggctgctagcccatatgtcgcgtggatgtccgtgacggccgggcaggctgaacttaccgctgcccaggtaagagttgctgcagccgcgtacgaaacagcgtatggcctgacggttcccccgcccgtgatcgcggaaaaccgcgccgaactgatgattctcatcgcgactaacctcctcggccagaacaccccggcaattgcggtgaacgaggcggaatatggtgaaatgtgggcgcaggacgcagccgccatgttcggctacgcagcggcaacagcgacagcaacagcaaccctgctgcctttcgaagaagcgcctgaaatgaccagtgccggcggcctgttggaacaggcagcagccgtggaggaagcgtccgatactgctgccgccaatcagctgatgaacaatgtgcctcaggcgctgcagcaactggcccagcctacccagggcaccaccccgagcagcaaactcggcgggctgtggaaaaccgtctccccgcaccgcagcccgatttctaatatggtgtccatggccaataatcatatgagcatgaccaatagcggcgtcagcatgaccaacactctgagcagcatgctgaaaggctttgccccggcggctgccgcgcaggccgtccagaccgccgcgcaaaatggtgtgcgcgctatgtcgtccctgggaagttcactcggttcttccggtctggggggtggtgtggcagccaacttaggtcgtgccgcgagcgttggcagcttatcggtaccacaggcatgggcagcagcgaaccaggctgtaacgccagcagcacgcgcgttgccgctaacctcgctaacctccgcggcagagcgtgggccgggccagatgcttggaggacttccagttgggcaaatgggggcacgtgcaggtggtgggctttcaggagtattgcgggtacctcctcgtccctatgtgatgccacattccccggctgctggtgatatcgcaccaccagcgctatctcaagatcggtttgcagattttccggcgctgccgctggaccctagcgccatggtagcgcaggtgggcccgcaggttgtcaatataaataccaagttaggttataataacgcggtcggcgctggcacgggcattgtcatcgatccgaatggtgtggttctgaccaacaatcatgtgatcgcgggcgcgaccgatattaacgcctttagtgtggggagtggacagacgtacggcgtggatgtcgttggctacgaccgaacacaggatgttgccgtgctgcagctccgtggtgccggtgggttaccgtcggcggcgattggtggaggcgtagccgtcggtgaaccagttgtagcaatgggcaattcgggcggacaaggggggacacctcgagcagtaccgggaagggtggtggccctgggccaaaccgttcaagccagcgatagtttgacgggagcagaggaaacgttaaatggtctgattcagtttgatgcggccatccaaccgggcgatgctggcggtccggttgttaatggcctgggccaggttgtgggtatgaacacggcggcttctggtagcctcctgtgggtgctgggcctgttggtcggtctgttgttgttactgttaggcctgggggcgtgttacacctggcgtcgccgccgcgtctgcaaatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 440 - protein, 778 aa, synthetic construct
MKAILVVMLYTFTTANAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLAIYSTVASSLVLVVSLGAISFWMCSNGSLQCRICI

SEQ ID NO: 441 - RNA, 2527 nt, synthetic construct
  source 1..2527: mol_type = other RNA
acatttgcttctgacacaactgtgttcactagcaacctcaaacagccaccatgaaagccattttggtcgtgatgctgtacaccttcaccactgccaacgcgatgaccgctgcctcggataacttccagttatcacaggggggccaaggcttcgcaatcccgataggtcaggcgatggcaatcgccggacaaattcgttctgggggtggttcgcctaccgtgcacatcggccccaccgcctttctgggtttaggtgtggtggataacaatggcaacggcgcgagggtgcagcgcgtggtcggaagcgcccctgccgccagtctcggtatttccacgggcgacgtcatcacggctgtcgatggtgcaccgatcaattcagccacggccatggccgacgcattaaacgggcatcacccgggtgacgtgatttcggtcacttggcagacgaaatccggtggcacccgtacgggcaacgtcaccctggctgaaggccctcccgccgaatttatggttgacttcggcgcactgccgccagaaatcaatagtgctcggatgtatgctggcccggggagcgcgtctcttgtggcggcagcccaaatgtgggatagtgtcgcctcggatctcttttctgcggcgagcgcgtttcaatcggttgtgtgggggcttacggtagggtcgtggattggctcctccgctggcctgatggttgccgcagcgagtccgtatgtggcgtggatgtcagtaactgcaggacaggcggaacttacggcagcacaggtacgcgtagcagctgcagcgtacgaaacggcgtacggcctgaccgtcccaccgccagtgatcgcggaaaatcgcgccgagttaatgatcctgattgccactaacctcttgggccagaacacccccgctatcgcggtaaacgaggccgaatacggtgaaatgtgggcgcaggatgcggcagcgatgttcggttatgcggcggctacggcgactgctacagcaaccctgttaccgtttgaggaggcacctgagatgaccagcgcaggtggcctgctggaacaagccgctgcggttgaagaagcctcagacaccgccgctgcgaatcagctgatgaataatgttccgcaagcgctgcagcagctggcgcagccgacccagggtacaacgccgagctccaaactgggcggtctgtggaagaccgtgtcacctcatcgtagcccgatttctaatatggtgagcatggccaataatcatatgagcatgaccaattcgggcgtctctatgacgaacacactcagctccatgctcaaaggttttgctccggccgctgcagctcaggcggttcagaccgcggcgcagaatggcgttcgtgcgatgagctccctagggtcaagcttgggaagctctggtttgggtggcggtgtagctgcgaacttgggtcgtgccgcgtcagtgggctcattgtcggtaccgcaggcttgggcagctgcgaatcaagcggtaactcccgccgcacgtgccctgcctcttacatctctgacaagtgctgccgaacgcggtccgggtcaaatgcttggaggcttacctgtgggtcagatgggcgcaagagcgggtggtgggctgagtggcgtcttgcgtgtgccgccacgcccatatgttatgccccatagtccagcggccggagatattgccccgccagcgttgtcccaagatcgctttgcggattttccggctctgcctctggatccgtccgccatggttgcgcaggttggcccgcaggtggtgaacattaacaccaagctgggctataacaatgcggtcggcgctggcaccggcatcgttattgatccgaatggcgtcgtcctgaccaacaaccacgtgattgcgggcgccaccgatatcaatgcattctcggtcggtagcggtcagacttatggcgtggacgtggttggttatgaccgcacacaggatgtggccgtactgcagctgcgcggggcgggcggtctgccatccgcagcaattggaggtggcgttgcagtaggagaacccgttgtggcaatggggaacagcggcggacaaggtgggactccacgggcggttccggggcgagttgtagcgctaggacagacagtgcaggcaagcgacagtcttacgggggcagaagagaccctgaatggcctgatccaatttgatgcggcgatacaaccgggcgatgccggcggaccggtggtgaacggtctgggccaagttgtcggtatgaacaccgcggcctctggtagcctcgcaatttacagtacggttgccagcagtttagtgctggtcgtttctttaggtgcgatttctttctggatgtgctcgaacggcagcctgcaatgtcgtatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 442 - protein, 776 aa, synthetic construct
MKAILVVMLYTFTTANAMTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDAGGPVVNGLGQVVGMNTAASGSLIPIAVGGALAGLVLIVLIAYLVGRKRSHGYQTI

SEQ ID NO: 443 - RNA, 1388 nt, Mycobacterium tuberculosis
  source 1..1388: mol_type = genomic RNA
acatttgcttctgacacaactgtgttcactagcaacctcaaacagccaccatgaaggccattttagttgtcatgttatatacgttcaccacggcgaacgcgatgacggccgccagcgataatttccaactaagccagggcggccaaggctttgcaattcccatcggacaggcgatggccatcgctggccaaattcgtagtggcggcggaagcccgacggtgcatatcggcccgactgcttttcttggcctgggcgtcgtagataataacggtaacggggcccgagtacagcgcgtggtaggttcggcgccagcggcatcactgggtatatcgacgggggacgttatcacagctgtggatggggcgccaattaacagcgcaaccgcgatggccgatgccctgaatggccatcatccgggggatgtgattagtgtcacgtggcaaaccaagtcaggtggcacccgcaccggcaacgttaccctggccgagggtccgcctgcggagtttatggtggatttcggcgcactgccgccggaaatcaactctgcgcgtatgtatgctggtccgggcagcgcctcactggtcgcggcggctcaaatgtgggactcagtggctagtgatctcttctcagcagcgtcggccttccaatcggtggtttggggcttgactgtgggtagttggatcggcagttccgctggtctcatggtggccgccgcgtcgccgtatgtggcttggatgagtgtgacagcaggtcaggccgaacttactgccgctcaggtaagagtcgccgcggctgcgtatgaaactgcctatggcctgaccgttccgccgccggtcattgccgaaaatcgcgctgaactcatgattctcattgccacgaatctgctgggtcaaaatacgcctgccatcgcagtgaatgaagccgagtacggcgagatgtgggcccaggatgcggcggcaatgttcggttacgctgcagcaacggcgactgcaaccgccacccttttgccgtttgaagaagcgcctgaaatgacgtcggcaggcggcctgttggaacaggcggctgccgtcgaagaggccagcgataccgcggcagccaaccagttaatgaataacgtccctcaagccctgcagcagctggcccagcctactcagggtaccactccgagctctaaattaggtggcctctggaaaacggtgagcccccaccgctccccgattagtaacatggtctccatggcgaacaaccatatgagcatgacgaattcgggtgtcagcatgacaaacaccctgtcgtccatgctgaaaggctttgcgccggcggctgcggcgcaggcggttcaaaccgcagctcagaacggagtgcgtgcaatgtcttctctgggctcc

SEQ ID NO: 444 - protein, 338 aa, Mycobacterium tuberculosis
MQLVDRVRGAVTGMSRRLVVGAVGAALVSGLVGAVGGTATAGAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGA

SEQ ID NO: 445 - protein, 347 aa, synthetic construct
MGFLPKLLLLASFFPAGQAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGSLLWVLGLLVGLLLLLLGLGACYTWRRRRVCK

SEQ ID NO: 446 - RNA, 1234 nt, synthetic construct
  source 1..1234: mol_type = other RNA
acatttgcttctgacacaactgtgttcactagcaacctcaaacagccaccatgggctttctcccgaaactgctgttactggcaagctttttcccagccggccaggcgttctcccgcccgggattgcctgtagaatatctgcaagtaccgagcccgtctatgggtcgggacatcaaagtgcagtttcaaagtggtggcgccaatagccccgcgctgtacctgcttgatgggctgcgtgcgcaagacgattttagcggctgggacattaatacgccggcgtttgaatggtatgatcagtcggggctgtcggttgtgatgcctgtgggcgggcagtctagcttttatagcgattggtatcaaccagcctgtggaaaagccggctgccagacctacaaatgggaaacctttctgactagtgagctgccgggatggctgcaggctaaccgtcatgtcaaaccaaccggcagtgcggtggtcggtttatcgatggctgcatctagcgcgctgacgctggccatttatcacccgcagcagttcgtatacgcaggcgcgatgtccggtttgttagatccctcacaggctatgggtccaaccctcattggtctggccatgggtgatgctggcggctataaagcctcagatatgtggggtccgaaagaggacccggcttggcaacgtaacgaccctttgttgaacgttgggaagttaatcgcgaacaacacgcgagtttgggtttactgtggcaatggcaaaccatcggatctggggggtaataacctgccggccaagtttttggaaggttttgtgcgtacatccaacatcaaattccaggatgcctacaacgcgggcggcggccataatggtgttttcgacttcccggatagtggtactcactcatgggaatactggggcgcacaacttaatgcaatgaagccggatcttcagcgcgcactgggtgcaacacctaataccggaccggcgccccagggcgcgggctctctgctatgggtcttaggtttactggtcggtctacttctcctgctgctcgggttgggagcgtgctatacctggagacgccgccgcgtgtgcaaatgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 447 - protein, 350 aa, synthetic construct
MKAILVVMLYTFTTANAFSRPGLPVEYLQVPSPSMGRDIKVQFQSGGANSPALYLLDGLRAQDDFSGWDINTPAFEWYDQSGLSVVMPVGGQSSFYSDWYQPACGKAGCQTYKWETFLTSELPGWLQANRHVKPTGSAVVGLSMAASSALTLAIYHPQQFVYAGAMSGLLDPSQAMGPTLIGLAMGDAGGYKASDMWGPKEDPAWQRNDPLLNVGKLIANNTRVWVYCGNGKPSDLGGNNLPAKFLEGFVRTSNIKFQDAYNAGGGHNGVFDFPDSGTHSWEYWGAQLNAMKPDLQRALGATPNTGPAPQGAGSLAIYSTVASSLVLVVSLGAISFWMCSNGSLQCRICI

SEQ ID NO: 448 - RNA, 1243 nt, synthetic construct
  source 1..1243: mol_type = other RNA
acatttgcttctgacacaactgtgttcactagcaacctcaaacagccaccatgaaagccattctggtggtgatgttatatacctttaccaccgctaacgctttctcacgccctgggttgccggtagaatacctgcaagtgcccagtccgtcgatgggacgggacattaaggttcaatttcaatctggaggtgcgaattcgcccgcgctgtacttactggatggcctgcgtgcccaggatgattttagcggttgggatattaataccccggcctttgagtggtatgaccagagtggcctgtcggttgtgatgcctgtcggcggccagtccagcttttattccgactggtaccagccggcatgtggcaaagctggctgccaaacttataaatgggaaacttttctcacgagtgaactgccaggctggctacaagcgaaccgccacgttaaaccgacggggagtgccgtagtcggcttgagtatggctgcgagctctgcgctgactctcgcgatctatcatccacagcagtttgtttatgcgggtgctatgagcggacttctggatcccagccaggccatgggaccgacgttaatcgggcttgccatgggcgatgccgggggttacaaagcaagcgatatgtggggtcctaaagaggatccggcgtggcagcgaaacgatccgctgctcaatgttgggaagctgatcgcgaataatacccgtgtctgggtctattgcggtaacggcaaaccatcggacctaggtggtaataacctgccggccaagttcctggaaggttttgtacgcaccagcaatataaaattccaggacgcgtacaacgcaggcggtggccataatggcgtgttcgacttcccggattcaggcacgcactcttgggaatactggggcgcccagttgaacgctatgaaaccggatctgcaacgtgcgttaggtgcaacacctaacacagggccggcgccacagggtgcaggtagcctggcaatttattcaaccgtggcgtcttcactggtgcttgtggtctccctgggtgcaatctccttctggatgtgttccaacggcagcttgcagtgccgcatttgtatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 449 - protein, 374 aa, Mycobacterium tuberculosis
VKIRLHTLLAVLTAAPLLLAAAGCGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISS

SEQ ID NO: 450 - protein, 159 aa, Mycobacterium tuberculosis
VKRGLTVAVAGAAILVAGLSGCSSNKSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCS

SEQ ID NO: 451 - protein, 554 aa, synthetic construct
MKAILVVMLYTFTTANACGSKPPSGSPETGAGAGTVATTPASSPVTLAETGSTLLYPLFNLWGPAFHERYPNVTITAQGTGSGAGIAQAAAGTVNIGASDAYLSEGDMAAHKGLMNIALAISAQQVNYNLPGVSEHLKLNGKVLAAMYQGTIKTWDDPQIAALNPGVNLPGTAVVPLHRSDGSGDTFLFTQYLSKQDPEGWGKSPGFGTTVDFPAVPGALGENGNGGMVTGCAETPGCVAYIGISFLDQASQRGLGEAQLGNSSGNFLLPDAQSIQAAAAGFASKTPANQAISMIDGPAPDGYPIINYEYAIVNNRQKDAATAQTLQAFLHWAITDGNKASFLDQVHFQPLPPAVVKLSDALIATISSGGSGGGGSGGCSSNKSTTGSGETTTAAGTTASPGAASGPKVVIDGKDQNVTGSVVCTTAAGNVNIAIGGAATGIAAVLTDGNPPEVKSVGLGNVNGVTLGYTSGTGQGNASATKDGSHYKITGTATGVDMANPMSPVNKSFEIEVTCSGSLAIYSTVASSLVLVVSLGAISFWMCSNGSLQCRICI

SEQ ID NO: 452 - RNA, 1855 nt, synthetic construct
  source 1..1855: mol_type = other RNA
acatttgcttctgacacaactgtgttcactagcaacctcaaacagccaccatgaaggcgattttggtcgttatgctttataccttcaccaccgcgaacgcttgcgggtcaaaaccgccaagcggcagcccggaaactggagctggggcaggaactgtagcaacaacaccagcatcatcccccgtgacgcttgcggagacgggatcaaccctgttgtatccgctctttaacctgtggggcccagcctttcacgagcgctatcccaatgttaccattacggctcaggggactggatctggtgcaggcatcgcacaggcggcagcaggcacagttaatataggcgcgagcgatgcatatctctcggaaggcgatatggctgcgcataagggcctgatgaacattgctttagcgattagcgcgcagcaggtcaattacaacctgccgggtgtcagcgaacatctgaagctgaatggcaaagtcctcgccgccatgtaccaaggtaccatcaaaacctgggatgatccccaaatcgcggccctgaaccctggcgttaacctgccaggtaccgcggtggtgcctttgcatcgttcggacggctcgggtgacacttttctgttcacgcagtacttatctaaacaagatccggaaggttgggggaaatcaccgggcttcggtactacggtggactttcctgcagtaccgggtgctctgggggaaaacggaaatggtgggatggtgaccgggtgtgcggaaacaccgggttgtgtggcctatattggtatttcgtttctggatcaggctagccagcgtggcttaggcgaagcgcaattaggtaattccagcggcaactttttactaccggacgcacagagcatccaagcagccgccgcggggtttgcgtcaaaaaccccggccaatcaggcaattagcatgattgatggcccggcgccagacggctaccccatcatcaactatgaatatgcgattgttaataatcggcagaaagacgcggcgaccgcgcagacccttcaggcgtttctccactgggcgatcaccgatggcaacaaggcctcgttcctggatcaagtgcattttcaaccgctaccgccggccgtagtaaaactgtcggatgcgctgatcgccaccatcagtagtggtggcagtggtggtggaggtagtggcggatgctcaagcaacaaaagtacaacgggatctggcgagacgactaccgcagcgggcacaacggcttcccctggtgcggcaagtggccctaaagttgtgatcgacggcaaggatcaaaatgtgacggggtctgtcgtttgtaccacggccgccggtaacgtcaacatcgccattgggggtgcagccactggcatagcggccgtcctgaccgatggcaatccgccagaagtgaaatcggttggcttgggcaatgttaacggcgtaacgcttggctacacgtccggtaccggtcagggcaacgcttccgctactaaagacgggtctcactataaaatcaccggcaccgctaccggtgtcgatatggccaatccgatgtctccggttaacaaatccttcgagattgaagtgacgtgcagcggttctctggctatttacagtaccgtagccagctctctggtgttggtggtgtcgctgggtgccattagcttctggatgtgcagcaatggttccctgcagtgtcgcatttgcatttgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 453-500 - skipped (48 entries)

SEQ ID NO: 501 - RNA, 50 nt, synthetic construct
  source 1..50: mol_type = other RNA
acatttgcttctgacacaactgtgttcactagcaacctcaaacagccacc

SEQ ID NO: 502 - RNA, 134 nt, synthetic construct
  source 1..134: mol_type = other RNA
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 503-504 - skipped (2 entries)

SEQ ID NO: 505 - RNA, 1796 nt, synthetic construct
  source 1..1796: mol_type = other RNA
acatttgcttctgacacaactgtgttcactagcaacctcaaacagccaccatggaactgctgatcctgaaggccaacgccatcaccactattctgaccgccgtgaccttttgcttcgcctccggccagaacatcacagaggaattctaccagagcacctgttctgccgtcagcaagggctacctgtctgccctgagaacaggctggtacacctctgtgatcaccatagagctgagtaacatcaaagagaacaagtgtaatggaaccgatgcaaaggtgaaactgattaaacaggagctggacaagtacaagaacgccgtcaccgagctgcagctactgatgcagagcacccccgccaccggctcaggcagcgccatctgcagcggcgtcgccgtgtgcaaggtgctgcacctggaaggcgaagtgaacaaaatcaagtctgctctgctgtccacaaacaaggctgttgtgagcctgagcaatggcgtgagcgtgctgacattcaaggtgctagatctgaaaaactacatcgataagcaactgctgcctatcctgaacaagcagagctgcagcatcagcaacatcgagacagtgatcgaatttcagcagaaaaacaaccggctgctggaaatcacaagagagttcagcgtgaacgccggagtgaccacccctgtgtctacctacatgctgacaaatagcgagctgctgagcctgatcaacgacatgcctattaccaacgaccagaagaagctgatgagcaacaacgtgcagatcgtgcggcagcaatcatactctatcatgtgcatcatcaaggaagaagtgctggcttacgtggtgcaactgcctctgtacggcgtaatcgacacaccatgctggaagctgcacaccagccctctgtgcacaaccaacaccaaggaaggctccaacatctgtctgaccaggacagatagaggctggtattgtgacaacgccggcagcgtgagcttcttcccccaggccgagacatgcaaagtgcagagcaatagagtgttttgcgacaccatgaacagcagaaccctgccatctgaggtgaacctgtgtaacgtggacatcttcaaccccaagtacgactgcaagatcatgaccagcaaaacagatgtgagttctagcgtcatcaccagcctgggtgccatcgtatcttgctacggcaaaaccaagtgcaccgccagcaataagaatcggggaatcatcaagacattcagcaacggctgcgactacgtttctaataagggagtggataccgtgtctgtgggaaataccctgtactgtgtgaacaagcaggagggcaagagcctgtatgtgaagggcgagcctatcatcaacttctacgatcctctggtgttccctagcgacgagttcgacgctagcatcagccaggtgaatgagaagatcaaccagtccctcgccttcatcagaaagtccgacgagctgctccacaacgtgaacgctggcaagagcacaacaaacatcatgatcaccacaattatcatcgtgatcatcgtgatcctgctgagcctgatcgccgtgggcctgctgctgtactgcaaggccagaagcacccccgtgaccctgtccaaggaccagctgagcggcatcaacaacatcgcctttagcaactgataataggctcgctttcttgctgtccaatttctattaaaggttcctttgttccctaagtccaactactaaactgggggatattatgaagggccttgagcatctggattctgcctaataaaaaacatttattttcattgc

SEQ ID NO: 506 - RNA, 1798 nt, synthetic construct
  source 1..1798: mol_type = other RNA
acatttgcttctgacacaactgtgttcactagcaacctcaaacagccaccatggagctgctgatcctgaaggccaacgccatcaccaccattctgacagccgtcaccttctgctttgcctctggtcagaacatcaccgaggagttctaccagagcacctgcagcgccgtgagcaaggggtacttgtccgccctgcggaccggctggtacaccagcgtcattaccattgagctctccaacatcaaggagaacaagtgcaatggtactgacgccaaggtgaagctgatcaagcaggagctggacaagtacaagaatgccgtcacggagctgcagctgctcatgcagagcacacccgccaccgggtccggcagcgccatctgcagcggcgtggccgtctgcaaggtgctgcacctggagggcgaggtgaacaagatcaagtctgctctgctgagcaccaacaaggcagtggtgagcctgtcaaatggggtgagcgtgttgaccttcaaggtgctcgacctgaagaattacattgacaagcagctgctgccaattctgaacaagcagagttgcagcatctcgaacatcgagaccgtgatcgagttccagcagaagaacaatcggctgctggagatcaccagggagttctccgtgaacgccggggtgaccacccccgtgagcacatacatgctgaccaatagcgagctgctgtctctgatcaacgacatgcccatcaccaacgaccagaagaagctgatgagcaacaacgtgcagatcgtgagacagcagagctacagcattatgtgcatcatcaaggaggaggtcctggcttacgtggtgcagctgccgctgtacggcgtgatcgataccccttgttggaagctgcacactagtccactgtgcacaaccaacaccaaggagggcagcaacatctgcctgacgaggacggaccggggctggtattgcgacaatgccggctccgtgtccttcttccctcaggcagagacctgcaaggtgcagtccaaccgggtgttctgtgacaccatgaacagccggacgctgccctccgaggtgaacctgtgcaatgtggacatcttcaatcccaagtacgattgcaagatcatgaccagcaagaccgatgtgtcctccagcgtgatcacctcgctgggggccatcgtgtcttgctacggcaagaccaagtgtacagcttccaacaagaacaggggtatcatcaagacgttcagcaacggctgcgactacgtgagcaacaagggggtggacaccgtgagcgtggggaacaccctgtactgcgtgaacaagcaggaggggaagagcctgtacgtgaagggggagcccatcatcaacttttacgaccccctggtcttcccctccgacgagttcgacgccagtatcagccaggtgaatgagaagatcaatcagagcctggctttcatcagaaagtctgatgagctgctgcacaatgtgaacgccgggaagagcaccaccaacatcatgatcacgaccatcatcatcgtgatcatcgtgatcctgctcagcctgatcgctgtgggtctgctgctgtactgcaaggcccgcagtacaccagtgaccctcagcaaggatcagctgagcgggatcaacaacatcgccttctccaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 507 - RNA, 1798 nt, synthetic construct
  source 1..1798: mol_type = other RNA
acatttgcttctgacacaactgtgttcactagcaacctcaaacagccaccatggagttgctgatcctgaaggctaatgccatcaccacgattctgacggccgtgacattttgcttcgcctccgggcagaacatcacggaggagttctatcagtctacttgttctgcagtcagcaaggggtacttgtccgccctgcggaccggctggtacactagcgtcattaccattgagctctccaacatcaaggagaacaagtgcaatggtactgacgctaaggtgaagctgatcaagcaggagctggacaagtacaagaacgccgtgaccgagctgcagctgctgatgcagagcactcccgccaccgggagtggctctgccatctgcagcggggtcgcggtgtgcaaggtcctgcaccttgagggtgaggtcaacaagatcaagtctgctctgctgagcaccaacaaggcggtggtcagcctgagcaacggggtctctgtcctcaccttcaaggtgctggacctgaagaactacatcgataagcagctgctgccaattctgaacaagcagagttgcagcatcagcaatatcgagacggtgatcgagttccagcagaagaacaatcggctgctggagatcacccgggagttcagcgtgaacgccggggtgacaaccccggtgtccacctacatgctgaccaactccgagcttctgtctctgatcaatgatatgcctatcaccaatgatcagaagaagctgatgtccaacaatgtgcagatcgtgcgccagcagtcctactccataatgtgcatcattaaggaggaggtgctggcgtacgtggtgcagcttccactgtacggggtgatcgacaccccgtgctggaagctgcacacctctcctttgtgtaccaccaacacaaaggaggggtccaacatctgcctgacgaggacggaccggggctggtattgcgacaatgccggctccgtgtccttcttccctcaggcagagacctgcaaggtgcagagtaaccgggtgttctgtgacaccatgaacagccggactctgccctcggaggtcaatctgtgcaatgtggacatcttcaaccccaagtatgactgcaagatcatgacaagtaagactgatgtgagctcctccgtgatcaccagcctgggggcgatcgtgtcttgctacggcaagacaaagtgcacggcctccaacaagaatcgtgggatcattaagaccttcagtaatggttgtgattatgtgtccaacaagggggtggacactgtcagcgttggcaacacgctgtactgcgtgaacaagcaggagggcaagtcgctctacgtgaagggcgagccgattattaacttctacgatcccctggtgttccctagcgacgagttcgacgctagcatcagccaggtgaacgagaagattaatcagtcgctcgccttcatcagtaagagcgacgagctcctgcacaatgtcaacgctggcaagtccaccaccaacatcatgatcacaaccattatcatcgtgatcattgtgatcctgctcagcctgatcgctgtgggtctgctgctgtactgcaaggcccgcagtacaccagtgaccctcagcaaggatcagctgagcgggatcaacaatatcgcgttcagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 508 - RNA, 1798 nt, synthetic construct
  source 1..1798: mol_type = other RNA
acatttgcttctgacacaactgtgttcactagcaacctcaaacagccaccatggagttgctgatcctgaaggcgaatgctattaccaccatcctgacagctgtgaccttctgttttgcctccgggcagaacatcacggaggagttctatcagtctacttgttctgcagtcagcaaggggtacctgtccgcgttgcgcacggggtggtacacctccgtgatcacgattgagctctctaacatcaaggagaataagtgtaacggcactgacgccaaggtcaagctgatcaagcaggagctggacaagtataagaacgctgtgaccgagttacagcttcttatgcagtccactcctgctacaggcagcggctccgctatctgtagcggagtcgctgtctgtaaggtcttgcacctggagggcgaggtgaacaagatcaagtccgccttgctctccaccaacaaggccgtggtcagcttgtccaatggcgtcagtgtccttacatttaaggtccttgatctcaagaattacattgataagcagctgctgcctattctgaataagcagagctgctctatcagtaatattgagacggtgatcgagttccagcagaagaacaatcggctgctggagatcaccagagagttcagcgtgaacgcgggggtgaccaccccggtcagcacctacatgctgaccaactctgagctgttgtcgctcatcaacgacatgccgatcaccaatgatcagaagaagttgatgagcaacaacgttcagatcgtgcgccagcagtcctactccataatgtgcatcattaaggaggaggtgctggcgtacgtggtgcagcttccactgtacggggtgatcgacaccccgtgctggaagctgcacacctctcctttgtgtaccaccaacacaaaggaggggtccaacatctgcctgacgaggacggaccggggctggtattgcgacaatgccggctccgtgtccttcttccctcaggcagagacctgcaaggtgcagagtaaccgggtgttctgtgacaccatgaacagccggactctgccctcggaggtcaacctgtgcaacgtggacatcttcaaccccaagtatgactgcaagatcatgacaagtaagactgatgtgagctcctccgtgatcacgtcactgggggcgatcgtgtcttgctacggcaagaccaagtgcaccgccagcaataagaacagggggatcattaagacatttagtaatggttgtgattatgtgtccaacaagggggtggacactgtcagcgttggcaacacgctgtactgcgtgaacaagcaggagggcaagtcgctctacgtgaagggcgagccgattattaacttctatgacccgctggtgttcccgtcggacgagttcgacgcgagcatcagccaggtcaacgagaagattaatcagtcgctcgcctttatacgtaagagcgacgaactcctgcacaatgtcaacgctggcaagtccaccaccaacatcatgatcacaaccattattatcgtgatcatagtgatcctcctgtctcttattgcggtgggcctcttgctgtactgcaaggcacggtcgaccccagtgacgctgagcaaggaccagctgtcaggaattaataacatcgccttcagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 509 - protein, 321 aa, Human respiratory syncytial virus A
MSKTKDQRTAKTLERTWDTLNHLLFISSCLYKLNLKSIAQITLSILAMIISTSLIIAAIIFIASANHKVTPTTAIIQDATNQIKNTTPTHLTQNPQLGISLSNLSGTTSQSTTILASTTPSAESTPQSTTVKIINTTTTQILPSKPTTKQRQNKPQNKPNNDFHFEVFNFVPCSICSNNPTCWAICKRIPNKKPGKKTTTKPTKKPTLKTTKKDPKPQTTKPKGVLTTKPTGKPTINTTKTNSRTTLLTSNTKGNPEHTSQKETIHSTTSEGYPSPSQVYTTSDQEETLHSTTSEGYPSPSQVYTTSEYLSQSLSSSNTTK

SEQ ID NO: 510 - protein, 48 aa, Human respiratory syncytial virus A
RQNKPQNKPNNDFHFEVFNFVPCSICSNNPTCWAICKRIPNKKPGKKT

SEQ ID NO: 511 - protein, 310 aa, Human respiratory syncytial virus B
MSKNKNQRTARTLEKTWDTLNHLIVISSCLYKLNLKSIAQIALSVLAMIISTSLIIAAIIFIISANHKVTLTTATVQTIKNHTEKNITTYLTQVSPERVSPSKQPTATPPIHTNSATISPNTKSETHHTTTQTKGTISTPTQNNKPSTKPRPKNPPKKDDYHFEVFNFVPCSICGNNQLCKSICKTIPSNKPKKKPTTKPTNKPPTKTTNKRDPKTLAKTPKKETTINPTKKPTPKTTERDTSTPQSTVLDITTSKHTERDTSTSQSIALDTTTSKHTTQQQSLYSTIPENTPNSTQTPTASEPSTSNSI

SEQ ID NO: 512 - protein, 43 aa, Human respiratory syncytial virus B
KNPPKKDDYHFEVFNFVPCSICGNNQLCKSICKTIPSNKPKKK

SEQ ID NO: 513 - protein, 612 aa, synthetic construct
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFLGSGGGSGGRQNKPQNKPNNDFHFEVFNFVPCSICSNNPTCWAICKRIPNKKPGKKTGSGGGSGGKNPPKKDDYHFEVFNFVPCSICGNNQLCKSICKTIPSNKPKKK

SEQ ID NO: 514 - RNA, 2029 nt, synthetic construct
  source 1..2029: mol_type = other RNA
acatttgcttctgacacaactgtgttcactagcaacctcaaacagccaccatggaactcctcatcctgaaagccaacgccatcaccacaattctcactgctgtgaccttctgttttgcctcgggacagaacatcaccgaggagttctaccagtccacatgctccgctgtgtcgaagggctacctgtcggcgctgaggactggctggtacacctccgtgattaccatcgaactgtcaaacatcaaggagaacaagtgcaacggtactgacgcgaaggtcaagctgattaagcaggaattggacaagtacaagaacgccgtgactgagctccagctgctgatgcagtcaacccctgcgacgggaagcgggtccgcaatctgctccggagtggccgtgtgcaaagtgctgcatctggagggcgaagtcaacaagatcaagtcggccctgctgagcaccaacaaggccgtggtgtccttgagtaacggagtctccgtcctgaccttcaaagttctggacctgaagaattacatcgataagcagctcctgcccatcctcaacaagcagagctgctccatttcgaacatcgaaactgtgatcgagtttcagcaaaagaacaaccggctgctggaaatcaccagagagtttagcgtgaacgcaggagtgactacccccgtgtctacctacatgctaaccaactcagaactgcttagcttgatcaatgacatgccgatcactaacgatcagaagaagcttatgtccaacaacgtgcaaatcgtgcgccagcaatcctactcgattatgtgcattatcaaggaagaggtgctggcctatgtggtgcagctcccgctgtacggcgtgattgacactccttgctggaagctgcacacctcccccctgtgtaccacgaataccaaggaggggtccaatatctgcctgacccggaccgaccgcggatggtactgcgacaacgccggttcggtgtccttcttcccacaagccgagacttgcaaagtccagtccaatcgcgtgttctgcgacactatgaactcccggaccttgccgtccgaagtgaacctgtgtaacgtggacatcttcaaccctaaatacgactgcaagattatgacttccaagaccgacgtcagcagctcagtgatcaccagcctgggcgccatcgtgtcgtgttatggaaagaccaagtgcactgcgtccaacaagaacagaggcatcatcaagaccttcagcaacggctgcgattacgtcagcaacaagggggtggataccgtgtccgtgggaaacactctctactgtgtgaacaagcaggagggaaagtccctctacgtcaagggcgaaccgatcatcaacttctacgatcccctggtgttcccatccgacgagttcgacgcctccatttcacaagtcaacgaaaagatcaaccagtctctggcgttcatccggaagtcggatgaactcctttccgccattggtggatacattcctgaggctcctcgcgatggccaggcctacgtcaggaaagacggagaatgggtgctgctttcaaccttcctgggatccgggggtggctcaggcggcagacaaaacaagccccagaacaagccgaacaacgacttccactttgaagtgttcaatttcgtaccgtgctcgatttgttccaacaaccccacttgctgggcaatctgcaagcggatcccaaacaagaagcctggaaagaaaaccgggagcggcggaggttccggagggaagaacccgcctaagaaggacgactaccacttcgaggtgttcaacttcgtcccgtgttcgatctgcggcaacaatcagctgtgcaagtcaatttgcaagaccatcccctccaacaagcccaagaagaagtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 515 - protein, 165 aa, synthetic construct
MSKTKDQRTAKTLERTWDTLNHLLFISSCLYKLNLKSIAQITLSILALIISTSLIIAAIIFIAGGSRQNKPQNKPNNDFHFEVFNFVPCSICSNNPTCWAICKRIPNKKPGKKTGSGGGSGGKNPPKKDDYHFEVFNFVPCSICGNNQLCKSICKTIPSNKPKKK

SEQ ID NO: 516 - RNA, 688 nt, synthetic construct
  source 1..688: mol_type = other RNA
acatttgcttctgacacaactgtgttcactagcaacctcaaacagccaccatgagcaagaccaaggaccagcggaccgccaaaaccctcgaaaggacttgggatacccttaatcatctgctgttcatttcctcgtgcttatacaagctgaacctgaagtccattgcacaaattaccctgtcaattctggccctcatcatctccacctccctgatcattgccgctatcatcttcatcgcgggcggtagcagacaaaacaagccccagaacaagccgaacaacgacttccactttgaagtgttcaatttcgtaccgtgctcgatttgttccaacaaccccacttgctgggcaatctgcaagcggatcccaaacaagaagcctggaaagaaaaccgggagcggcggaggttccggagggaagaacccgcctaagaaggacgactaccacttcgaggtgttcaacttcgtcccgtgttcgatctgcggcaacaatcagctgtgcaagtcaatttgcaagaccatcccctccaacaagcccaagaagaagtgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 517-800 - skipped (284 entries)

SEQ ID NO: 801 - protein, 1010 aa, synthetic construct
MAAPGSARRPLLLLLLLLLLGLMHCASASASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVIRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKADSNNSSKSLQSAGFTAGLTYSQLMTLKDAMLQLDPNAKTWMDIEGRPEDPVEIALYQPSSGCYIHFFREPTDLKQFKQDAKYSHGIDVTDLFATQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVLGPGPGTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDLIPIAVGGALAGLVLIVLIAYLVGRKRSHAGYQTI

SEQ ID NO: 802 - RNA, 3223 nt, synthetic construct
  source 1..3223: mol_type = other RNA
acatttgcttctgacacaactgtgttcactagcaacctcaaacagccaccatggccgcccccgggagcgctaggcgccctcttctgctcttgctgttgctgctgcttctggggctgatgcattgcgcatcagcaagcgcctccaaggagatcaagtcgttcctgtggacccagtccctccggcgggagctgtcgggctactgctcgaacatcaagctccaagtcgtgaaagacgcgcaggccctgctgcacggactggatttctcggaagtctccaacgtccaaagattgatgcggaaagaaaggagggacgacaatgatctgaagaggctccgcgacctcaaccaggcggttaacaacctggtggaactgaagtcgacccagcaaaagagcattctgcgcgtgggcaccctcacctccgacgaccttctgatcctggccgccgatctggagaagttgaagtccaaggtcatccgcaccgaacggccgctgtccgccggggtgtacatgggaaacctgtcaagccagcagctggatcagcgcagagcacttcttaacatgatcggaatgtccggtggaaatcagggtgccagagcaggccgggacggagtcgtgcgggtctgggacgtgaagaacgcggagctgctgaacaaccagttcggcaccatgccgtcactgaccctggcctgtctcacaaagcagggccaagtggacctgaatgacgcagtgcaggcccttacggacctgggcctgatctacaccgctaagtacccgaataccagcgacctggatagactgactcagtcgcatccaattctgaatatgatcgacaccaagaagtcctcgctgaacatttccggttataacttctccttgggagcggccgtgaaggccggagcgtgcatgctggacggcgggaacatgctggaaaccatcaaagtgtccccgcaaaccatggacggaatcctcaagtccatcctgaaagtcaagaaggccctgggaatgttcatttctgacaccccgggagagaggaacccatacgagaatatcctgtacaagatttgtctgagcggggacggctggccgtacattgcgagccggactagcatcaccggtagagcctgggagaacaccgtggtcgaccttgaatccgatggaaagccacagaaggcagactccaacaactcttccaagtccctgcaaagcgccggattcaccgcgggactcacctactcccaactcatgaccctgaaggatgcaatgctccagctcgacccaaacgctaagacttggatggacatcgaaggccggcccgaagatcccgtggaaattgccctctaccaaccctcaagcggttgctacatccacttcttccgcgaaccgactgacctgaaacagtttaagcaggacgcaaagtactcccacggtatcgatgtgaccgacctctttgctacccaacctggcctgacctccgccgtgatcgatgccctgccccgcaacatggtcatcacttgtcaaggcagtgatgatatcaggaagctgctcgagagccagggtagaaaggatatcaagctgatcgacatcgcgctgagcaagactgacagccgcaaatacgaaaacgctgtgtgggaccagtacaaggacttgtgccacatgcacacaggggtggtggtcgaaaagaagaagcggggaggaaaggaggagatcaccccacattgcgccctgatggactgcattatgttcgacgcggccgtgtcaggaggactcaacacttccgtgctccgggccgtgctgccccgggacatggtgttcagaacttccactcctcgcgtggtgctgggcccgggacctggaacctcactgtacaagggggtgtacgaactgcagaccctggaattgaacatggaaactctgaacatgaccatgcctctctcctgtaccaagaacaattcccaccactacattatggtcggaaacgaaactgggctcgagctgaccctcaccaacacctccataatcaaccacaagttctgcaacctttctgatgcccacaagaagaacctatacgaccatgccctgatgtccattatctccaccttccacctctcaatccccaacttcaatcaatacgaagcgatgtcctgtgatttcaacggcggaaagatctcggtgcagtacaacctctcgcattcatacgccggagatgccgccaaccactgcggtactgtggccaacggggtgctccagacctttatgaggatggcgtggggaggatcctacatcgccttggactccggacgcggcaactgggactgcatcatgaccagctaccagtacctgattattcagaacaccacctgggaggaccactgccagttctcgcgcccttcgccgattgggtaccttggcctcctgtcgcaaagaaccagagacatctacatcagccgccggttactgggaacttttacttggaccctttcggattccgagggaaaggacacgcccggcggttactgcctgacgcggtggatgctgattgaagctgaactcaagtgtttcggcaacaccgccgtggccaagtgcaacgagaagcacgacgaagagttctgcgatatgctgcgactgttcgatttcaacaagcaggctattcagcggctgaaggccgaggcccagatgagcattcagctgatcaacaaggccgtgaacgccctgattaacgatcagctcattatgaagaaccatctgcgggacatcatgggcatcccttactgcaactactccaagtattggtacctgaaccacactaccactggccgcaccagcctcccgaagtgctggctcgtgtccaacggatcctatctgaacgaaacccacttttccgacgacattgagcagcaggccgacaacatgatcaccgagatgttgcagaaggagtacatggagcgccaggggaaaactcccctgggactggtggaccttatccctattgctgtgggcggtgccttggctggcctggtactgatcgtgctgatcgcgtatctcgtgggccggaaacggtcacacgccgggtaccagactatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 803 - protein, 1024 aa, synthetic construct
MRVTAPRTLILLLSGALALTETWASASKEIKSFLWTQSLRRELSGYCSNIKLQVVKDAQALLHGLDFSEVSNVQRLMRKERRDDNDLKRLRDLNQAVNNLVELKSTQQKSILRVGTLTSDDLLILAADLEKLKSKVIRTERPLSAGVYMGNLSSQQLDQRRALLNMIGMSGGNQGARAGRDGVVRVWDVKNAELLNNQFGTMPSLTLACLTKQGQVDLNDAVQALTDLGLIYTAKYPNTSDLDRLTQSHPILNMIDTKKSSLNISGYNFSLGAAVKAGACMLDGGNMLETIKVSPQTMDGILKSILKVKKALGMFISDTPGERNPYENILYKICLSGDGWPYIASRTSITGRAWENTVVDLESDGKPQKADSNNSSKSLQSAGFTAGLTYSQLMTLKDAMLQLDPNAKTWMDIEGRPEDPVEIALYQPSSGCYIHFFREPTDLKQFKQDAKYSHGIDVTDLFATQPGLTSAVIDALPRNMVITCQGSDDIRKLLESQGRKDIKLIDIALSKTDSRKYENAVWDQYKDLCHMHTGVVVEKKKRGGKEEITPHCALMDCIMFDAAVSGGLNTSVLRAVLPRDMVFRTSTPRVVLGPGPGTSLYKGVYELQTLELNMETLNMTMPLSCTKNNSHHYIMVGNETGLELTLTNTSIINHKFCNLSDAHKKNLYDHALMSIISTFHLSIPNFNQYEAMSCDFNGGKISVQYNLSHSYAGDAANHCGTVANGVLQTFMRMAWGGSYIALDSGRGNWDCIMTSYQYLIIQNTTWEDHCQFSRPSPIGYLGLLSQRTRDIYISRRLLGTFTWTLSDSEGKDTPGGYCLTRWMLIEAELKCFGNTAVAKCNEKHDEEFCDMLRLFDFNKQAIQRLKAEAQMSIQLINKAVNALINDQLIMKNHLRDIMGIPYCNYSKYWYLNHTTTGRTSLPKCWLVSNGSYLNETHFSDDIEQQADNMITEMLQKEYMERQGKTPLGLVDGIVAGLAVLAVVVIGAVVAAVMCRRKSSGGKGGSYSQAACSDSAQGSDVSLTA

SEQ ID NO: 804 - RNA, 3265 nt, synthetic construct
  source 1..3265: mol_type = other RNA
acatttgcttctgacacaactgtgttcactagcaacctcaaacagccaccatgcgggtcactgcgccgagaaccctcattcttctcctatccggggcgctggctctcaccgaaacctgggcaagcgcctccaaggagatcaagtcgttcctgtggacccagtccctccggcgggagctgtcgggctactgctcgaacatcaagctccaagtcgtgaaagacgcgcaggccctgctgcacggactggatttctcggaagtctccaacgtccaaagattgatgcggaaagaaaggagggacgacaatgatctgaagaggctccgcgacctcaaccaggcggttaacaacctggtggaactgaagtcgacccagcaaaagagcattctgcgcgtgggcaccctcacctccgacgaccttctgatcctggccgccgatctggagaagttgaagtccaaggtcatccgcaccgaacggccgctgtccgccggggtgtacatgggaaacctgtcaagccagcagctggatcagcgcagagcacttcttaacatgatcggaatgtccggtggaaatcagggtgccagagcaggccgggacggagtcgtgcgggtctgggacgtgaagaacgcggagctgctgaacaaccagttcggcaccatgccgtcactgaccctggcctgtctcacaaagcagggccaagtggacctgaatgacgcagtgcaggcccttacggacctgggcctgatctacaccgctaagtacccgaataccagcgacctggatagactgactcagtcgcatccaattctgaatatgatcgacaccaagaagtcctcgctgaacatttccggttataacttctccttgggagcggccgtgaaggccggagcgtgcatgctggacggcgggaacatgctggaaaccatcaaagtgtccccgcaaaccatggacggaatcctcaagtccatcctgaaagtcaagaaggccctgggaatgttcatttctgacaccccgggagagaggaacccatacgagaatatcctgtacaagatttgtctgagcggggacggctggccgtacattgcgagccggactagcatcaccggtagagcctgggagaacaccgtggtcgaccttgaatccgatggaaagccacagaaggcagactccaacaactcttccaagtccctgcaaagcgccggattcaccgcgggactcacctactcccaactcatgaccctgaaggatgcaatgctccagctcgacccaaacgctaagacttggatggacatcgaaggccggcccgaagatcccgtggaaattgccctctaccaaccctcaagcggttgctacatccacttcttccgcgaaccgactgacctgaaacagtttaagcaggacgcaaagtactcccacggtatcgatgtgaccgacctctttgctacccaacctggcctgacctccgccgtgatcgatgccctgccccgcaacatggtcatcacttgtcaaggcagtgatgatatcaggaagctgctcgagagccagggtagaaaggatatcaagctgatcgacatcgcgctgagcaagactgacagccgcaaatacgaaaacgctgtgtgggaccagtacaaggacttgtgccacatgcacacaggggtggtggtcgaaaagaagaagcggggaggaaaggaggagatcaccccacattgcgccctgatggactgcattatgttcgacgcggccgtgtcaggaggactcaacacttccgtgctccgggccgtgctgccccgggacatggtgttcagaacttccactcctcgcgtggtgctgggcccgggacctggaacctcactgtacaagggggtgtacgaactgcagaccctggaattgaacatggaaactctgaacatgaccatgcctctctcctgtaccaagaacaattcccaccactacattatggtcggaaacgaaactgggctcgagctgaccctcaccaacacctccataatcaaccacaagttctgcaacctttctgatgcccacaagaagaacctatacgaccatgccctgatgtccattatctccaccttccacctctcaatccccaacttcaatcaatacgaagcgatgtcctgtgatttcaacggcggaaagatctcggtgcagtacaacctctcgcattcatacgccggagatgccgccaaccactgcggtactgtggccaacggggtgctccagacctttatgaggatggcgtggggaggatcctacatcgccttggactccggacgcggcaactgggactgcatcatgaccagctaccagtacctgattattcagaacaccacctgggaggaccactgccagttctcgcgcccttcgccgattgggtaccttggcctcctgtcgcaaagaaccagagacatctacatcagccgccggttactgggaacttttacttggaccctttcggattccgagggaaaggacacgcccggcggttactgcctgacgcggtggatgctgattgaagctgaactcaagtgtttcggcaacaccgccgtggccaagtgcaacgagaagcacgacgaagagttctgcgatatgctgcgactgttcgatttcaacaagcaggctattcagcggctgaaggccgaggcccagatgagcattcagctgatcaacaaggccgtgaacgccctgattaacgatcagctcattatgaagaaccatctgcgggacatcatgggcatcccttactgcaactactccaagtattggtacctgaaccacactaccactggccgcaccagcctcccgaagtgctggctcgtgtccaacggatcctatctgaacgaaacccacttttccgacgacattgagcagcaggccgacaacatgatcaccgagatgttgcagaaggagtacatggagcgccaggggaaaactcccctgggactggtggacgggattgtggctgggttggcggtcctggcggtggtggtgatcggtgccgtggtggcagccgtgatgtgcaggagaaagtcgtccggcgggaagggagggtcctacagccaagcggcctgttctgactccgcccagggctccgacgtgtcactgacagcctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 805-900 - skipped (96 entries)

SEQ ID NO: 901 - RNA, 1798 nt, synthetic construct
  source 1..1798: mol_type = other RNA
acatttgcttctgacacaactgtgttcactagcaacctcaaacagccaccatggaactgctgatcctgaaggccaacgccatcaccactattctgaccgccgtgaccttttgcttcgcctccggccagaacatcacagaggaattctaccagagcacctgttctgccgtcagcaagggctacctgtctgccctgagaacaggctggtacacctctgtgatcaccatagagctgagtaacatcaaagagaacaagtgtaatggaaccgatgcaaaggtgaaactgattaaacaggagctggacaagtacaagaacgccgtcaccgagctgcagctactgatgcagagcacccccgccaccggctcaggcagcgccatctgcagcggcgtcgccgtgtgcaaggtgctgcacctggaaggcgaagtgaacaaaatcaagtctgctctgctgtccacaaacaaggctgttgtgagcctgagcaatggcgtgagcgtgctgacattcaaggtgctagatctgaaaaactacatcgataagcaactgctgcctatcctgaacaagcagagctgcagcatcagcaacatcgagacagtgatcgaatttcagcagaaaaacaaccggctgctggaaatcacaagagagttcagcgtgaacgccggagtgaccacccctgtgtctacctacatgctgacaaatagcgagctgctgagcctgatcaacgacatgcctattaccaacgaccagaagaagctgatgagcaacaacgtgcagatcgtgcggcagcaatcatactctatcatgtgcatcatcaaggaagaagtgctggcttacgtggtgcaactgcctctgtacggcgtaatcgacacaccatgctggaagctgcacaccagccctctgtgcacaaccaacaccaaggaaggctccaacatctgtctgaccaggacagatagaggctggtattgtgacaacgccggcagcgtgagcttcttcccccaggccgagacatgcaaagtgcagagcaatagagtgttttgcgacaccatgaacagcagaaccctgccatctgaggtgaacctgtgtaacgtggacatcttcaaccccaagtacgactgcaagatcatgaccagcaaaacagatgtgagttctagcgtcatcaccagcctgggtgccatcgtatcttgctacggcaaaaccaagtgcaccgccagcaataagaatcggggaatcatcaagacattcagcaacggctgcgactacgtttctaataagggagtggataccgtgtctgtgggaaataccctgtactgtgtgaacaagcaggagggcaagagcctgtatgtgaagggcgagcctatcatcaacttctacgatcctctggtgttccctagcgacgagttcgacgctagcatcagccaggtgaatgagaagatcaaccagtccctcgccttcatcagaaagtccgacgagctgctccacaacgtgaacgctggcaagagcacaacaaacatcatgatcaccacaattatcatcgtgatcatcgtgatcctgctgagcctgatcgccgtgggcctgctgctgtactgcaaggccagaagcacccccgtgaccctgtccaaggaccagctgagcggcatcaacaacatcgcctttagcaactgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 902 - RNA, 1798 nt, synthetic construct; identical to SEQ ID NO: 506
  source 1..1798: mol_type = other RNA

SEQ ID NO: 903 - RNA, 1798 nt, synthetic construct; identical to SEQ ID NO: 507
  source 1..1798: mol_type = other RNA

SEQ ID NO: 904 - RNA, 1798 nt, synthetic construct; identical to SEQ ID NO: 508
  source 1..1798: mol_type = other RNA

SEQ ID NO: 905 - protein, 566 aa, Alphainfluenzavirus influenzae
MKAILVVMLYTFTTANADTLCIGYHANNSTDTVDTVLEKNVTVTHSVNLLEDKHNGKLCKLRGVAPLHLGQCNIAGWILGNPECESLSTARSWSYIVETSNSDNGTCYPGDFINYEELREQLSSVSSFERFEIFPKTSSWPNHDSDNGVTAACSHAGARSFYKNLIWLVKKGKSYPKINQTYINDKGKEVLVLWGIHHPPTITDQESLYQNADAYVFVGTSRYSKKFKPEIAARPKVRDRAGRMNYYWTLVEPGDKITFEATGNLVAPRYAFTMEKEAGSGIIISDTPVHDCNATCQTPEGAINTSLPFQNVHPITIGKCPKYVRSTKLRLATGLRNVPSIQSRGLFGAIAGFIEGGWTGMVDGWYGYHHQNDQGSGYAADLKSTQNAIDKITNKVNSVIEKMNTQFTAVGKEFNHLEKRIENLNKKVDDGFLDVWTYNAELLVLLENERTLDYHDSNVKNLYEKVRHQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 906 - protein, 566 aa, Alphainfluenzavirus influenzae; identical to SEQ ID NO: 18

SEQ ID NO: 907 - protein, 566 aa, Alphainfluenzavirus influenzae
MKAIIALSNILCLVFAQKIPGNDNSTATLCLGHHAVPNGTIVKTITNDRIEVTNATELVQNSSIGKICNSPHQILDGGNCTLIDALLGDPQCDGFQNKEWDLFVERSRANSSCYPYDVPDYASLRSLVASSGTLEFKDESFNWTGVKQNGTSSACKRGSSNSFFSRLNWLTSLNNIYPAQNVTMPNKEQFDKLYIWGVHHPATDKNQFSLFAQSSGRITVSTKRSQQAVIPNIGSRPRVRDIPSRISIYWTIVKPGDILLINSTGNLIAPRGYFKIRSGKSSIMRSDAPIGECKSECITPNGSIPNDKPFQNVNRITYGACPRYVKQSTLKLATGMRNVPEKQTRGIFGAIAGFIENGWEGMVDGWYGFRHQNSEGRGQAADLKSTQAAIDQISGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 908-910 - skipped (3 entries)

SEQ ID NO: 911 - protein, 188 aa, unidentified
MFFKCNQVFTLVFLLLLYFNNIVYTHVDDIKNTSQKKITYDKYNKNKENMNNEKNDNKDNKDNIYNDNINNDNINNDNINNEDEYKFLSMKHYKDSLSNKLNNENDHMNYLIRKRKDNTQGSQHFNENIENNENVENNENIENNENNENIENIENNENNENNENIENNENNENNENSSIMNSESYNNI

SEQ ID NO: 912 - protein, 320 aa, synthetic construct
KNVNFLQYHFKELSNYNIANSIDILQEKEGHLDFVIIPHYTFLDYYKHLSYNSIYHKSSTYGKCIAVDAFIKKINETYDKVKSKCNDIKNDLIATIKKLEHPYDINNKNMNRTFKKMMDEYNTKKKKLIKCIKNHENDFNKICMDMKNYGTNLFEQLSCYNNNFCNTNGIRYHYDEYIHKLILSVKSKNLNKDLSDMTNILQQSELLLTNLNKKMGSYIYIDTIKFIHKEMKHIFNRIEYHTKIINDKTKIIQDKIKLNIWRTFQKDELLKRILDMSNEYSLFITSDHLRQMLYNTFYSKEKHLNNIFHHLIYVLQMKFN

SEQ ID NO: 913 - skipped

SEQ ID NO: 914 - RNA, 1891 nt, synthetic construct
  source 1..1891: mol_type = other RNA
acatttgcttctgacacaactgtgttcactagcaacctcaaacagccaccatgaaaactatcatcgccctctccaacatactctgcttggtgttcgcacaaaagatccctggaaacgacaatagcaccgcaaccctgtgtctcggacatcacgcggtgccgaacgggaccattgtcaagaccattaccaatgatcgcatcgaagtgacaaacgccaccgagctggtgcagaactcgtccatcggagagatctgcggcagcccgcatcagatcctggacggcggcaactgcactctgatcgatgccctcctgggtgatcctcagtgtgacggcttccaaaacaaggagtgggatctgttcgtggagcggagccgggccaacagcaactgttacccctacgatgtgcctgactacgcctcacttcggtccctggtcgcatcctccgggacgctggaattcaaaaacgagtcgttcaactggacgggagtgaaacagaacggcacctccagtgcttgcatccggggctcctcctcatcctttttctcgcgactgaactggcttacctcgctgaacaatatctacccggcgcagaacgtcaccatgcccaacaaggagcaatttgacaagctctacatttggggggtgcaccaccccgacactgacaagaaccagatctccctgtttgcccaaagctcaggtcgcattactgtgtccaccaagaggtcccagcaggccgtgatcccaaacattggaagcagaccgagaatccgcggcatcccatcccggatctcaatctactggactattgtgaagccgggggacatcctgctgatcaactccaccggaaacctgattgccccgcgcggctatttcaagattcgcagcggaaagtcgtccattatgagatcggacgcgcctatcgggaagtgcaagagcgaatgtatcacccccaacggatccatccccaatgacaagccgttccagaacgtgaaccgcatcacttacggagcttgccctcgctacgtgaagcagtcaaccctcaaactggccaccggcatgcggaacgtgcccgaaaagcagaccagaggaattttcggggccatcgcgggtttcattgaaaatggatgggagggaatggtggacggttggtacggattcaggcaccaaaactccgagggtcggggccaggccgccgacctgaagtccacccaggccgcgattgaccagatcaacgggaagctgaacaggctgattggaaagaccaacgaaaagttccaccaaatcgagaaggaattctcggaggtcgaagggagagtccaggacctcgagaaatacgtggaggacactaagattgatctgtggtcttacaacgccgaactgttggtggccctggaaaaccagcacaccatcgatctgactgatagcgagatgaacaagctgttcgaaaagactaagaagcagttgcgcgagaacgccgaggacatgggaaatggttgcttcaagatctatcataagtgcgacaacgcctgcatcggcagcattcggaacgaaacctacgaccacaacgtctaccgggatgaagctctgaacaaccggttccagatcaagggagtggaactcaagtccggctacaaggactggatcctttggatctcgtttgctatgtcctgcttccttctctgtatcgccctgctgggcttcattatgtgggcgtgccagaagggaaacattaggtgcaatatctgcatctgataataggctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 915 - protein, 582 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINTEDAPGGPYEIGTSGSCLNITNGKGFFATMAWAVPKNKTATNPLTIEVPYICTEEEDQITVWGFHSDDETQMARLYGDSKPQKFTSSANGVTTHYVSQIGGFPNQTEDGGLPQSGRIVVDYMVQKSGKTGTITYQRGILLPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

(19) United States
(12) Patent Application Publication
(10) Pub. No.: US 2026/0284195 A1
(43) Pub. Date: Sep. 24, 2026

(54) IONIZABLE LIPIDS AND LIPOSOMAL NANOPARTICLE COMPOSITIONS

(71) Applicant: Akagera Medicines, Inc., Waltham, MA (US)

(72) Inventors: Daryl C. Drummond, Lincoln, MA (US); Dmitri Kirpotin, San Francisco, CA (US); Mark E. Hayes, Mill Valley, CA (US); Ross Fulton, Southborough, MA (US); Alexander Koshkaryev, Norwood, MA (US)

(73) Assignee: Akagera Medicines, Inc., Waltham, MA (US)

(21) Appl. No.: 19/575,567

(22) Filed: Mar. 23, 2026

Related U.S. Application Data
(60) Provisional application No. 63/775,688, filed on Mar. 21, 2025.
(60) Provisional application No. 63/869,090, filed on Aug. 22, 2025.
(60) Provisional application No. 63/944,616, filed on Dec. 19, 2025.
(60) Provisional application No. 64/007,071, filed on Mar. 16, 2026.

(51) Int. Cl. A61K 47/22; A61K 9/51; A61K 39/00; A61K 39/385; A61K 47/10; A61K 47/24; A61K 47/28

(52) U.S. Cl. CPC A61K 47/22; A61K 9/5123; A61K 39/385; A61K 47/10; A61K 47/24; A61K 47/28; A61K 2039/53

(57) ABSTRACT

Aspects of the present disclosure provide for improved compositions of bioreducible and/or hydrolysable ionizable lipid compounds, useful in the preparation of lipid nanoparticles (LNP) for the delivery of therapeutic nucleic acids and other compounds to cells. Anionic phospholipids, including a phosphatidyl-L-serine (PS) or a phosphatidylglycerol (PG) can be included in the lipid nanoparticles to increase the transfection efficiency in human dendritic cells. In some examples, the LNP compositions are vaccine compositions comprising mRNA encapsulated in lipid nanoparticles formed from an ionizable cationic lipid compound, cholesterol, an anionic phospholipid and a PEG conjugated lipid.

### RELATED APPLICATIONS

This patent application claims the benefit of and priority to U.S. Provisional Patent Application No. 63/775,688, filed Mar. 21, 2025, U.S. Provisional Patent Application No. 63/869,090, filed Aug. 22, 2025, U.S. Provisional Patent Application No. 63/944,616, filed Dec. 19, 2025, and U.S. Provisional Patent Application No. 64/007,071, filed Mar. 16, 2026, the entire content of each of which is incorporated herein by reference in their entirety.

### REFERENCE TO SEQUENCE LISTING

The application contains a Sequence Listing which has been submitted electronically in XML file format and is hereby incorporated by reference in its entirety. Said XML copy, created on Mar. 23, 2026, is titled 191016-011611_US_SL.xml and is 394,185 bytes in size.

### FIELD

Aspects of the present disclosure relate to ionizable lipids useful for the preparation of lipid nanoparticles (LNP) encapsulating a nucleic acid.

### BACKGROUND

Lipid nanoparticles (LNP) are used for the delivery of therapeutic nucleic acids to cells, including vaccines to deliver mRNA therapeutics. LNP formulations typically include an ionizable lipid, a sterol, and a phospholipid. Ionizable lipids in LNP compositions facilitate delivery of encapsulated nucleic acid by remaining mostly neutral at physiological pH, but becoming protonated in the acidic environment of endosomes after cellular uptake. Ionizable lipids that are hydrolysable or bioreducible offer desirable in vivo clearance properties for vaccine-focused lipid nanoparticle (LNP) delivery systems by enhancing both efficacy and safety. Hydrolysable lipids, which break down through controlled chemical or enzymatic cleavage, and bioreducible lipids, which degrade in response to intracellular reducing conditions, promote timely LNP disassembly after delivering the vaccine payload. There remains a need for ionizable lipids that are hydrolysable and/or bioreducible for use in the preparation of LNP formulations for therapeutic delivery of nucleic acids.

### SUMMARY

In some embodiments, this disclosure provides ionizable cationic lipid compounds with significantly improved hydrolysis (Example 4) with significantly improved mRNA transfection efficiency in dendritic cells from LNPs encapsulating the mRNA (Example 5), compared to previously disclosed ionizable cationic lipids such as KC3-OA, ALC-0315 and SM-102. See, e.g., Yuxiang R, et al., “Impact of ionizable lipid type on the pharmacokinetics and biodistribution of mRNA-lipid nanoparticles after intravenous and subcutaneous injection,” Journal of Controlled Release, v. 384, 2025, 113945, ISSN 0168-3659 (study of ALC-0315 and SM-102 properties) and publication WO2022/115645, disclosing ionizable cationic lipids including KC3-OA.

In some embodiments, an ionizable lipid compound consists of a dioxolane ring covalently bound to both (a) an alkylamine head group having a pKa of 6-7.5, and (b) a pair of identical geminal functionalized aliphatic chains at the 2-position of the dioxolane ring, each having 18-25 total carbon atoms and each consisting of (i) a 7-10 carbon linear aliphatic group covalently bound to the dioxolane ring and to (ii) an ester functional group, and (iii) a 10-20 carbon aliphatic chain covalently bound to the ester functional group. In some embodiments, the dioxolane ring is a 1,3-dioxolane ring and each of the functionalized aliphatic chains comprises two or more branched C₅ hydrocarbon monomer units such as isopentane (IPN) or 2-methylbut-2-ene (2 MB).

In some embodiments, a lipid nanoparticle (LNP) composition comprises a mRNA nucleic acid encapsulated in lipidic nanoparticles formed from the ionizable lipid disclosed herein at a N/P ratio of 3.5 to 8 relative to the mRNA nucleic acid in a total amount of 40-65 mol % of the total lipid content of the LNP composition; a sterol in a total amount of 25-45 mol % of the total lipid content of the LNP composition; one or more phospholipids in a total amount of phospholipids of 5-25 mol % of the total lipid content of the LNP composition; and a PEG-containing conjugated lipid in a total amount of 0.5-5.0 mol % of the total lipid content of the LNP composition. In some embodiments, the two or more phospholipids in the LNP composition comprise a total of 5 mol % of a phosphatidyl-L-serine (PS). In some embodiments, the LNP composition comprises the conjugated lipid in a total amount of 0.5-2.5 mol % of the total lipid content of the LNP composition. In some embodiments, the phospholipids consist of: 1,2-distearoyl-sn-glycero-3-phosphocholine (DSPC) in a total amount of 5 mol % of the total lipid content of the LNP composition, and a phosphatidyl-L-serine (DPPS), such as dipalmitoylphosphatidyl-L-serine (DPPS) or distearoylphosphatidyl-L-serine (DSPS), in a total amount of 5 mol % of the total lipid content of the LNP composition. In some embodiments, the PEG-containing conjugated lipid in the LNP is PEG(2000)-dimyristoylglycerol (PEG-DMG) or PEG(2000)-dilauroylglycerol (PEG-DLG).

In some embodiments, a lipid nanoparticle (LNP) composition comprises a mRNA nucleic acid encoding an antigenic protein encapsulated at a N/P ratio of 3.25-8 in lipidic nanoparticles consisting of: a KCn-X1 or KCn-X2 ionizable cationic lipid disclosed herein and cholesterol in a molar ratio of the ionizable cationic lipid to the cholesterol of from 65:35 to 45:55, X mol % of an anionic phospholipid such as DPPS (wherein X is 2.5-7.5 mol %), (10-X) mol % phosphocholine phospholipid such as DSPC, and 1.5-4 mol % of a PEG conjugated lipid such as PEG-DMG. In some embodiments, a lipid nanoparticle (LNP) composition comprises a mRNA nucleic acid encapsulated at a N/P ratio of 5.25 in lipidic nanoparticles consisting of: 56 mol % of the KC3-X1-PH12 ionizable cationic lipid, 5 mol % DPPS, 5 mol % DSPC, 1.5 mol % PEG-DMG and 32.5 mol % cholesterol. In some embodiments, a lipid nanoparticle (LNP) composition comprises a mRNA nucleic acid encapsulated at a N/P ratio of 5.25 in lipidic nanoparticles consisting of: 52 mol % of the KC3-X1-PH12 ionizable cationic lipid, 5 mol % DPPS, 5 mol % DSPC, 1.5 mol % PEG-DMG and 36.5 mol % cholesterol.

### BRIEF DESCRIPTION OF THE DRAWINGS

FIG. 1A is a synthetic scheme for preparing an exemplary ionizable cationic lipid designated KC3-X1-K.

FIG. 1B is a table of biodegradable ionizable cationic lipids with KC headgroups and fully saturated alkyl chains using X1 ester configuration.

FIG. 2A is a synthetic scheme for preparing an exemplary ionizable cationic lipid designated KC3-X1-AA.

FIG. 2B is a table of biodegradable ionizable cationic lipids with KC headgroups and unsaturated alkyl chains using X1 ester configuration.

FIG. 3A is a synthetic scheme for preparing an exemplary ionizable cationic lipid designated KC3-X1-PH5.

FIG. 3B is a synthetic scheme for preparing an exemplary ionizable cationic lipid designated KC3-X2-PH12.

FIG. 3C is a table of biodegradable ionizable cationic lipids with KC headgroups and saturated phytyl alkyl chains using X1 ester configuration.

FIG. 4A is a synthetic scheme for preparing an exemplary ionizable cationic lipid designated KC3-X1-PH1B.

FIG. 4B is a table of biodegradable ionizable cationic lipids with KC headgroups and unsaturated phytyl alkyl chains.

FIG. 5A is a synthetic scheme for preparing an exemplary ionizable cationic lipid designated KC3-X2-K9.

FIG. 5B is a table of biodegradable ionizable cationic lipids with KC headgroups and saturated alkyl chains using X2 ester configuration.

FIG. 6A is a synthetic scheme for preparing an exemplary ionizable cationic lipid designated KC3-X2-C(Trans-).

FIG. 6B is a table of biodegradable ionizable cationic lipids with KC headgroups and unsaturated alkyl chains using X2 ester configuration.

FIG. 7A is a synthetic scheme for preparing an exemplary ionizable cationic lipid designated KC3-X2-PH12.

FIG. 7B is a table of biodegradable ionizable cationic lipids with KC headgroups and saturated phytyl alkyl chains using X2 ester configuration.

FIG. 8A is a synthetic scheme for preparing an exemplary ionizable cationic lipid designated DODAP-X1-PH12.

FIG. 8B is a synthetic scheme for preparing an exemplary ionizable cationic lipid designated DODAP-X1-PH5.

FIG. 8C are synthetic schemes for preparing an exemplary ionizable cationic lipids designated MAE-X1-PH12 and MA-X1-PH12.

FIG. 8D are synthetic schemes for preparing an exemplary ionizable cationic lipid designated DMA3E-X1-PH12 and DMA4E-X1-PH12.

FIG. 8E is a synthetic scheme for preparing an exemplary ionizable cationic lipid designated SM-X1-PH12A.

FIG. 8F are synthetic schemes for preparing an exemplary ionizable cationic lipid designated SM-X1-PH12B and ALC-PHY12A.

FIG. 8G is a synthetic scheme for preparing an exemplary ionizable cationic lipid designated DODMA-X1-PH5

FIG. 8H are synthetic schemes for preparing an exemplary ionizable cationic lipid designated MAE-X2-PH12 and MA-X2-PH12

FIG. 8I is a synthetic scheme for preparing an exemplary ionizable cationic lipid designated DMA3E-X2-PH12

FIG. 8J is a table of biodegradable ionizable cationic lipids with alternative headgroups and saturated phytyl alkyl chains using X1 and X2 ester configurations.

FIG. 9A is a graph of the biodegradability of ionizable cationic lipids (ICLs) measured after 15 minutes in the presence of pancreatin enzymes (including trypsin, amylase and lipase) measured according to the assay of Example 5.

FIG. 9B is a graph of the biodegradability of ionizable cationic lipids (ICLs) measured after 3 hours in the presence of pancreatin enzymes (including trypsin, amylase and lipase) measured according to the assay of Example 5.

FIG. 9C is a graph showing the enzymatic degradation of various ionizable lipids.

FIG. 10A is a chart describing a study design for a study comparing T cell responses after prime/boost mRNA vaccination of CB6F1 mice with a synthetic TB antigen.

FIG. 10B shows the magnitudes of the splenic CD4 T cell responses in the study described in FIG. 10A.

FIG. 10C shows the magnitudes of the splenic CD8 T cell responses in the study described in FIG. 10A.

FIG. 11A is a chart showing the study design for a comparison of T cell and B cell responses in BALB/c after prime/boost vaccination with an influenza A H5 mRNA.

FIG. 11B is a graph showing the antibody titers after the first vaccination in the study described in FIG. 11A.

FIG. 11C show the titers after the second vaccination in the study described in FIG. 11A.

FIG. 11D is a graph showing the frequency of the CD4 T cell response in the study described in FIG. 11A.

FIG. 11E is a graph showing the frequency of the CD8 T cell response in the study described in FIG. 11A.

FIG. 12A is a table showing the study design for a study comparing T cell and B cell responses in BALB/c after prime/boost vaccination with an influenza A H5 mRNA.

FIG. 12B is a graph showing the antibody titers after the first vaccination in the study described in FIG. 12A.

FIG. 12C is a graph showing the titers after the second vaccination in the study described in FIG. 12A.

FIG. 12D is a graph showing the frequency of the CD4 T cell response and FIG. 12E shows the frequence of the CD8 T cell response in the study described in FIG. 12A.

FIG. 13A is a table of the study design comparing T cell and B cell responses in BALB/c after prime/boost vaccination with an influenza A H5 mRNA.

FIG. 13B is a graph showing the antibody titers after the first vaccination in the study described in FIG. 13A.

FIG. 13C is a graph showing the titers after the second vaccination in the study described in FIG. 13A.

FIG. 13D is a graph showing the frequency of the CD4 T cell response and FIG. 13E shows the frequence of the CD8 T cell response in the study described in FIG. 13A.

FIG. 14A-14J are graphs showing the concentrations of cytokine and chemokine analytes in the blood of BALB/c mice 6 hrs after i.m. vaccination with influenza A H5 mRNA-LNPs comprised with different ionizable lipids. FIG. 14A is a graph showing the concentration of IFN-gamma in the study of FIGS. 14A-14J. FIG. 14B is a graph showing the concentration of IL-6 in the study of FIGS. 14A-14J. FIG. 14C is a graph showing the concentration of TNF-alpha in the study of FIGS. 14A-14J. FIG. 14D is a graph showing the concentration of IFN-alpha in the study of FIGS. 14A-14J. FIG. 14E is a graph showing the concentration of IL-12p70 in the study of FIGS. 14A-14J. FIG. 14F is a graph showing the concentration of CCL2 in the study of FIGS. 14A-14J. FIG. 14G is a graph showing the concentration of CCL5 in the study of FIGS. 14A-14J. FIG. 14H is a graph showing the concentration of IL-1beta in the study of FIGS. 14A-14J. FIG. 141 is a graph showing the concentration of CXCL1 in the study of FIGS. 14A-14J. FIG. 14J is a graph showing the concentration of CXCL10 in the study of FIGS. 14A-14J.

FIGS. 15A-15I are graphs showing the concentrations of cytokine and chemokine analytes in the draining lymph nodes of BALB/c mice 6 hrs after i.m. vaccination with influenza A H5 mRNA-LNPs comprised with different ionizable lipids. FIG. 15A is a graph showing the concentration of IFN-gamma in the study of FIGS. 15A-15I. FIG. 15B is a graph showing the concentration of IL-6 in the study of FIGS. 15A-15I. FIG. 15C is a graph showing the concentration of TNF-alpha in the study of FIGS. 15A-15I. FIG. 15D is a graph showing the concentration of IFN-alpha in the study of FIGS. 15A-15I. FIG. 15E is a graph showing the concentration of IL-12p70 in the study of FIGS. 15A-15I. FIG. 15F is a graph showing the concentration of CCL2 in the study of FIGS. 15A-15I. FIG. 15G is a graph showing the concentration of CCL5 in the study of FIGS. 15A-15I. FIG. 15H is a graph showing the concentration of CXCL1 in the study of FIGS. 15A-15I. FIG. 151 is a graph showing the concentration of CXCL10 in the study of FIGS. 15A-15I.

FIG. 16A is a table showing a study design comparing T cell responses after prime/boost mRNA vaccination of CB6F1 mice with a synthetic TB antigen.

FIG. 16B is a graph showing the magnitudes of the splenic CD4 T cell responses in the study of FIG. 16A.

FIG. 16C is a graph showing the magnitudes of the splenic CD8 T cell responses in the study of FIG. 16A.

FIG. 17A is a table of the study design comparing of T cell and B cell responses in BALB/c after prime/boost vaccination with an influenza A H5 mRNA.

FIG. 17B is a graph showing the antibody titers after the first vaccination with a 0.1 μg dose in the study of FIG. 17A.

FIG. 17C is a graph showing the titers after the second vaccination with a 0.1 μg dose in the study of FIG. 17A.

FIG. 17D shows the antibody titers after the first vaccination with a 0.02 μg dose in the study of FIG. 17A.

FIG. 17E is a graph showing the titers after the second vaccination with a 0.02 μg dose in the study of FIG. 17A.

FIG. 17F is a graph showing the magnitudes of the CD4 T cell responses in the spleens of mice vaccinated with 0.1 μg in the study of FIG. 17A.

FIG. 17G is a graph showing the magnitudes of the CD4 T cell responses in the spleens of mice vaccinated with 0.02 μg in the study of FIG. 17A.

FIG. 17H is a graph showing the magnitudes of the CD8 T cell responses in the spleens of mice vaccinated with 0.1 μg in the study of FIG. 17A.

FIG. 171 is a graph showing the magnitudes of the CD8 T cell responses in the spleens of mice vaccinated with 0.02 μg in the study of FIG. 17A.

FIG. 18 is a table showing the study design comparing the in vivo immunogenicity of influenza H5 mRNA-LNPs with various ionizable lipids.

FIG. 19A is a graph showing the antibody titer results on study day 21 in the study of FIG. 18.

FIG. 19B is a graph showing the antibody titer results on study day 35 in the study of FIG. 18.

FIG. 19C is a graph showing the antibody titer results on study day 21 in the study of FIG. 18.

FIG. 19D is a graph showing the antibody titer results on study day 35 in the study of FIG. 18.

FIG. 19E is a graph showing the percentage cytokine+ of CD4 in the study of FIG. 18.

FIG. 19F is a graph showing the percentage cytokine+ of CD8 in the study of FIG. 18.

FIG. 20A is a table showing the study design comparing the in vivo immunogenicity of quadrivalent mRNA-LNPs comprised of KC3-X1-PH12 LNPs or KC3-X2-PH12 LNPs at various ICL mol % and N/P ratios.

FIG. 20B is a graph showing the total H1 high dose titers (0.03 mcg dose) in the study of FIG. 20A.

FIG. 20C is a graph showing the total H1 low dose titers (0.01 mcg dose) in the study of FIG. 20A.

FIG. 20D is a graph showing the total high dose H3 titers (0.045 mcg dose) in the study of FIG. 20A.

FIG. 20E is a graph showing low dose H3 titers (0.015 mcg dose) in the study of FIG. 20A.

FIG. 20F is a graph showing the total high dose B/Victoria titers (0.03 mcg dose) in the study of FIG. 20A.

FIG. 20G is a graph showing low dose B/Victoria titers (0.01 mcg dose) in the study of FIG. 20A.

FIG. 20H is a graph showing the total H5 titer in the study of FIG. 20A.

FIG. 21A is a table showing the study design for comparing the in vivo immunogenicity of a tuberculosis mRNA vaccines using KC3-X1-PH12 LNPs at various ICL mol % and after lyophilization and reconstitution.

FIG. 21B is a graph showing the total % cytokine+ CD4 response (1 mcg dose) in the study of FIG. 21A (KC3-X1-PH12).

FIG. 21C is a graph showing the total % cytokine+ CD4 response (0.3 mcg dose) in the study of FIG. 21A (KC3-X1-PH12).

FIG. 21D is a graph showing the total % cytokine+ CD4 response (0.1 mcg dose) in the study of FIG. 21A (KC3-X1-PH12).

FIG. 21E is a graph showing the total % cytokine+ CD8 response (1 mcg dose) in the study of FIG. 21A (KC3-X1-PH12).

FIG. 21F is a graph showing the total % cytokine+ CD8 response (0.3 mcg dose) in the study of FIG. 21A (KC3-X1-PH12).

FIG. 21G is a graph showing the total % cytokine+ CD8 response (0.1 mcg dose) in the study of FIG. 21A (KC3-X1-PH12). FIG. 21H is a graph showing the total % cytokine+ CD4 response (50 mol %, N/P=5.25) in the study of FIG. 21A (KC3-X1-PH12, Quadrivalent).

FIG. 21I is a graph showing the total % cytokine+ CD8 response (50 mol %, N/P=5.25) in the study of FIG. 21A (KC3-X1-PH12, Quadrivalent).

FIG. 22A is a table showing the study design for evaluating the in vivo pro-inflammatory profile of KC3-X1-PH5 and KC3-X1-PH12 LNP formulations.

FIG. 22B is a graph showing IFL-gamma concentrations in the tissue lysates of draining lymph nodes in the study of FIG. 22A.

FIG. 22C is a graph showing TNF-alpha concentrations in the tissue lysates of draining lymph nodes in the study of FIG. 22A.

FIG. 22D is a graph showing IL-12p70 concentrations in the tissue lysates of draining lymph nodes in the study of FIG. 22A.

FIG. 22E is a graph showing IL-6 concentrations in the tissue lysates of draining lymph nodes in the study of FIG. 22A.

FIG. 22F is a graph showing IL-1 beta concentrations in the tissue lysates of draining lymph nodes in the study of FIG. 22A.

FIG. 22G is a graph showing IFN-beta concentrations in the tissue lysates of draining lymph nodes in the study of FIG. 22A.

FIG. 22H is a graph showing IFN-gamma concentrations in the tissue lysates of draining lymph nodes in the study of FIG. 22A.

FIG. 221 is a graph showing CXCL1 concentrations in the tissue lysates of draining lymph nodes in the study of FIG. 22A.

FIG. 22J is a graph showing CCL2 concentrations in the tissue lysates of draining lymph nodes in the study of FIG. 22A.

FIG. 22K is a graph showing IFL-gamma concentrations in the blood serum of draining lymph nodes in the study of FIG. 22A.

FIG. 22L is a graph showing TNF-alpha concentrations in the blood serum of draining lymph nodes in the study of FIG. 22A.

FIG. 22M is a graph showing IL-12p70 concentrations in the blood serum of draining lymph nodes in the study of FIG. 22A.

FIG. 22N is a graph showing IL-6 concentrations in the blood serum of draining lymph nodes in the study of FIG. 22A.

FIG. 22O is a graph showing IL-1 beta concentrations in the blood serum of draining lymph nodes in the study of FIG. 22A.

FIG. 22P is a graph showing IFN-beta concentrations in the blood serum of draining lymph nodes in the study of FIG. 22A.

FIG. 22Q is a graph showing IFN-gamma concentrations in the blood serum of draining lymph nodes in the study of FIG. 22A.

FIG. 22R is a graph showing CXCL1 concentrations in the blood serum of draining lymph nodes in the study of FIG. 22A.

FIG. 22S is a graph showing CCL2 concentrations in the blood serum of draining lymph nodes in the study of FIG. 22A.

FIG. 23A is a schematic of mRNA constructs.

FIG. 23B is a schematic of mRNA constructs.

FIG. 24 is a graph showing the relationship between total methylene groups (n+m) in KC3 biodegradable ICLs and luciferase activity (as % of KC3-OA control lipid) at various (x) in Formula (I) ranging from 1-8.

FIG. 25 is a graph showing the minimum m terminal methylene length in structure Y needed to provide 10, 50, or 100% of the activity seen with the control KC3-OA ionizable lipid.

FIG. 26 is a graph showing the luciferase expression in murine dendritic cells transfected with luciferase mRNA LNPs as a function of post-transfection cell density (D).

FIG. 27 is a graph showing the correlation of luciferase expression in murine dendritic cells as a function of log(D).

FIG. 28A is a graph of total antibody titers obtained from the study of Example 13A.

FIG. 28B is a graph of total antibody titers obtained from the study of Example 13A.

FIG. 28C is a graph of total antibody titers obtained from the study of Example 13A.

FIG. 28D is a graph of total antibody titers obtained from the study of Example 13A.

FIG. 29A is a graph of total antibody titers obtained from the study of Example 13B.

FIG. 29B is a graph of total antibody titers obtained from the study of Example 13B.

FIG. 29C is a graph of total antibody titers obtained from the study of Example 13B.

FIG. 30A is a table describing the study of Example 14.

FIG. 30B is a graph of total antibody titers obtained from influenza H1 in the study of Example 14A.

FIG. 30C is a graph of total antibody titers obtained from influenza H1 in the study of Example 14A.

FIG. 30D is a graph of total antibody titers obtained from influenza H1 in the study of Example 14A.

FIG. 30E is a graph of total antibody titers obtained from influenza H3 in the study of Example 14A.

FIG. 30F is a graph of total antibody titers obtained from influenza H3 in the study of Example 14A.

FIG. 30G is a graph of total antibody titers obtained from influenza H3 in the study of Example 14A.

FIG. 30H is a graph of total antibody titers obtained from influenza B/Victoria in the study of Example 14A.

FIG. 301 is a graph of total antibody titers obtained from B/Victoria in the study of Example 14A.

FIG. 30J is a graph of total antibody titers obtained from B/Victoria in the study of Example 14A.

FIG. 31A is a graph of total antibody titers obtained from influenza H1 in the study of Example 14B.

FIG. 31B is a graph of total antibody titers obtained from influenza H1 in the study of Example 14A.

FIG. 31C is a graph of total antibody titers obtained from influenza H1 in the study of Example 14B.

FIG. 31D is a graph of total antibody titers obtained from influenza H3 in the study of Example 14A.

FIG. 31E is a graph of total antibody titers obtained from influenza H3 in the study of Example 14B.

FIG. 31F is a graph of total antibody titers obtained from influenza H3 in the study of Example 14B.

FIG. 31G is a graph of total antibody titers obtained from influenza B/Victoria in the study of Example 14B.

FIG. 31H is a graph of total antibody titers obtained from influenza B/Victoria in the study of Example 14B.

FIG. 31I is a graph of total antibody titers obtained from influenza B/Victoria in the study of Example 14B.

### DETAILED DESCRIPTION

It is to be understood that both the foregoing general description and the following detailed description are exemplary and explanatory only and are not restrictive of the compositions and methods of the present disclosure.

In some embodiments, ionizable cationic lipids (ICLs) are provided. In some embodiments, the present disclosure provides certain compounds useful as ionizable lipids in the preparation of lipid nanoparticles encapsulating a nucleic acid such as mRNA.

### 1. Ionizable Cationic Lipid Compounds

In some embodiments, an ionizable lipid compound comprises (a) an alkylamine head group having a pKa of 6-7.5, and (b) two or more linear or branched functionalized aliphatic chains, each having 18-25 total carbon atoms and each comprising (i) a saturated linear aliphatic group (X) covalently bound to the alkylamine headgroup and to (ii) a biodegradable or hydrolysable functional group such as an ester, and (iii) a linear or branched aliphatic chain (Y) optionally comprising one or more alkene groups, covalently bound to the bioreducible or hydrolysable functional group, further characterized in that (X) has a total of 8-10 carbon atoms, or (X)+(Y) has a total of 21-25 carbon atoms.

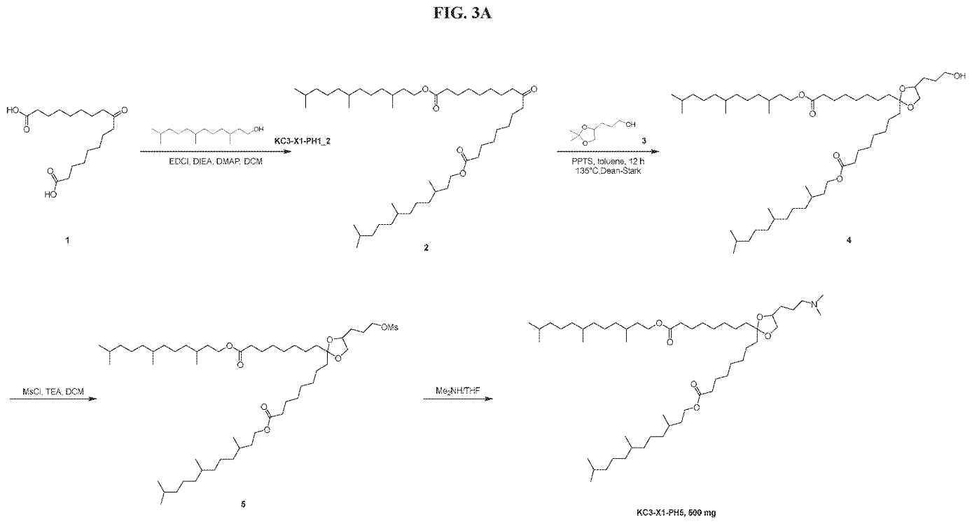

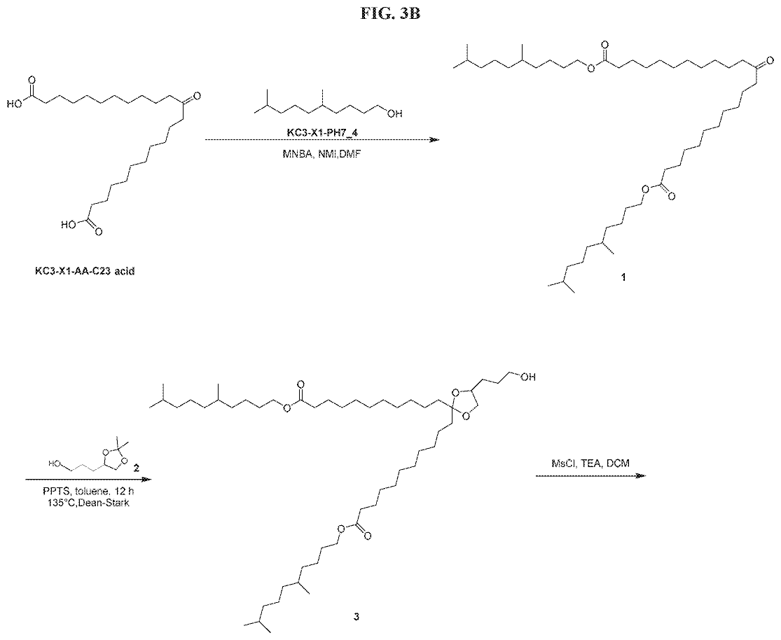

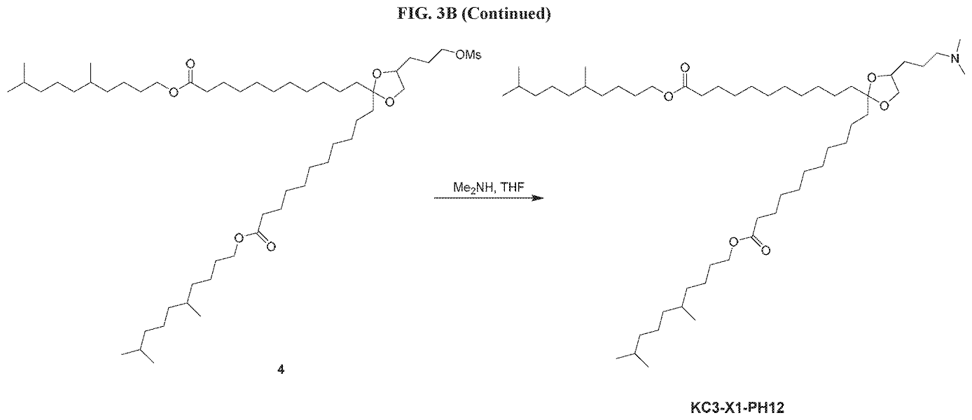

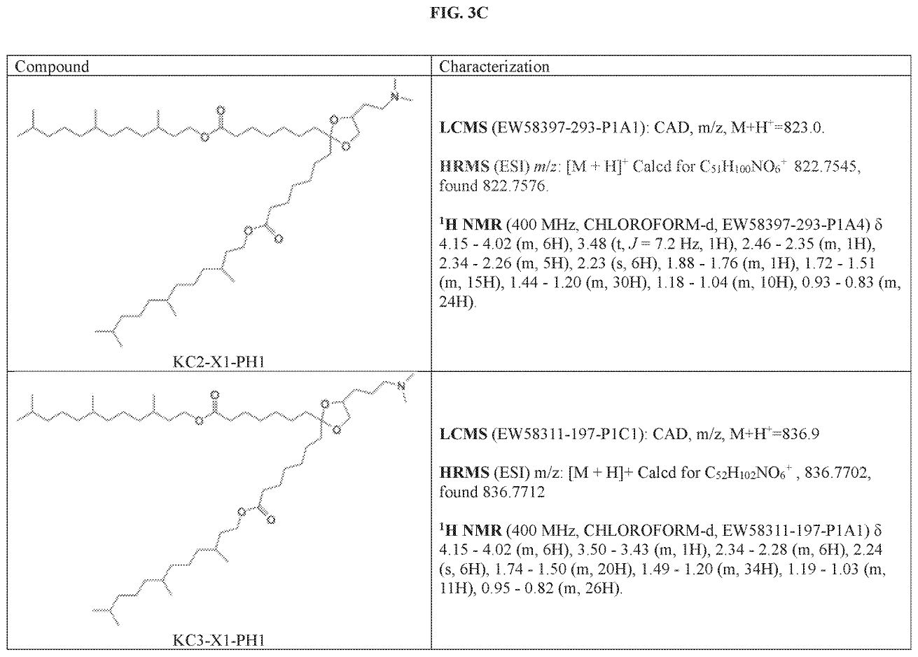

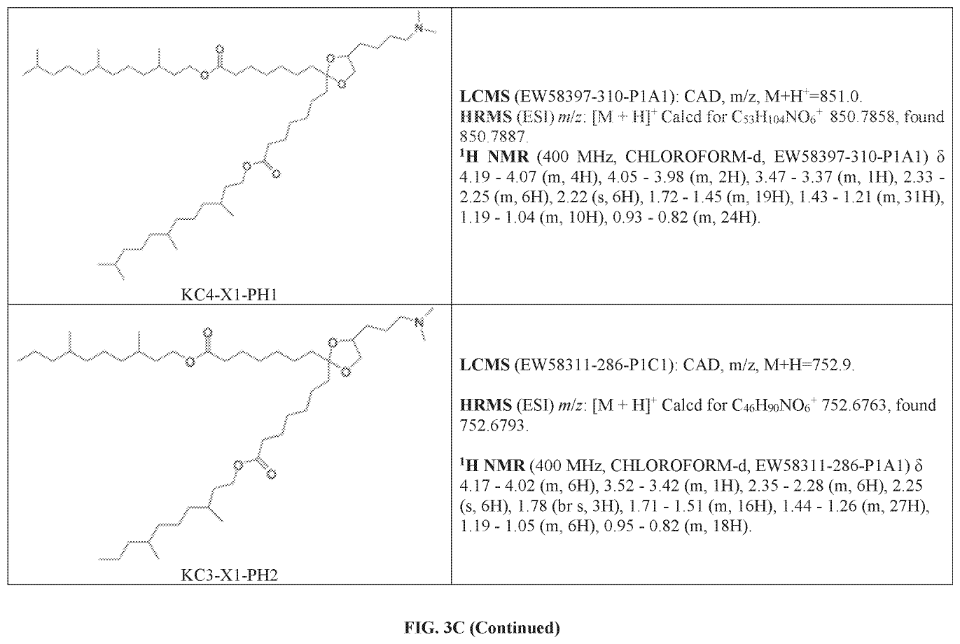

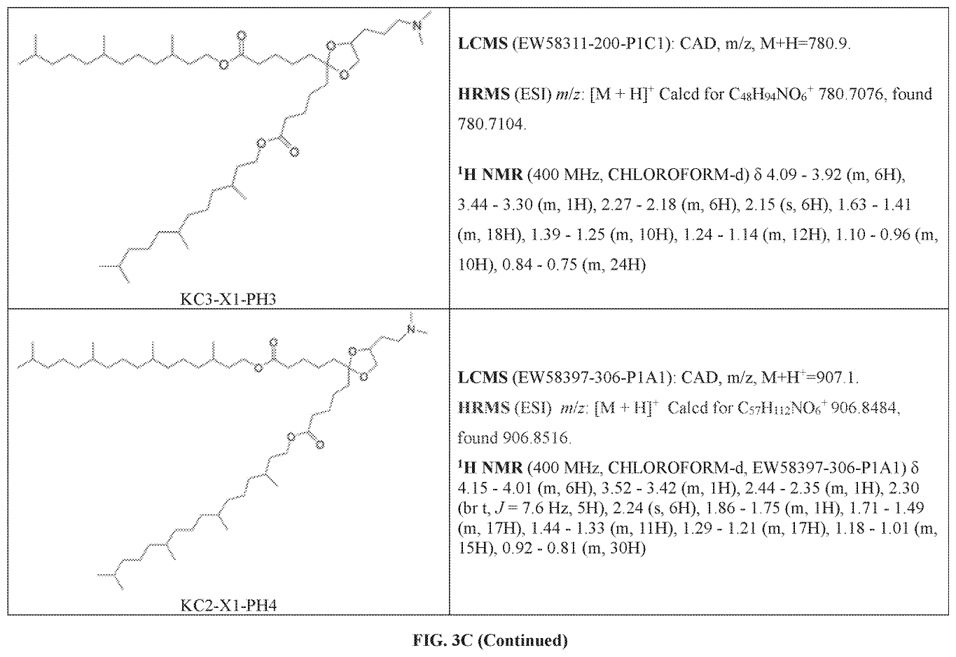

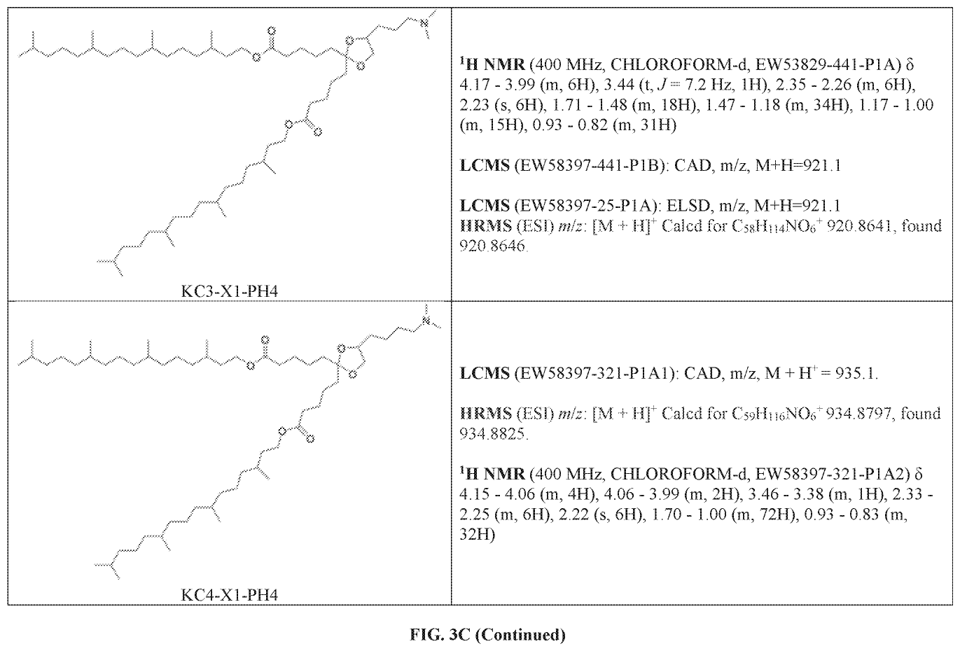

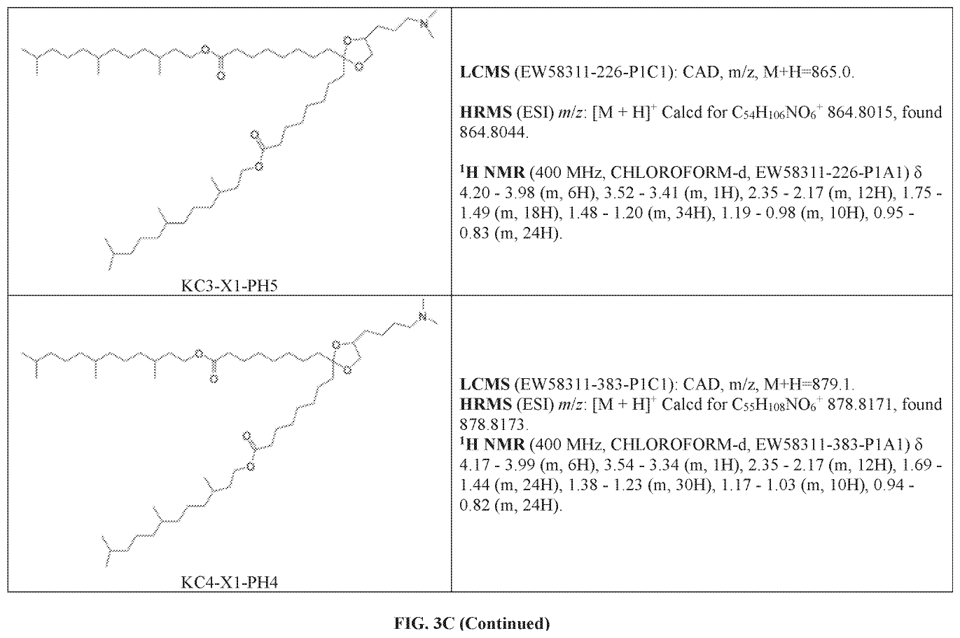

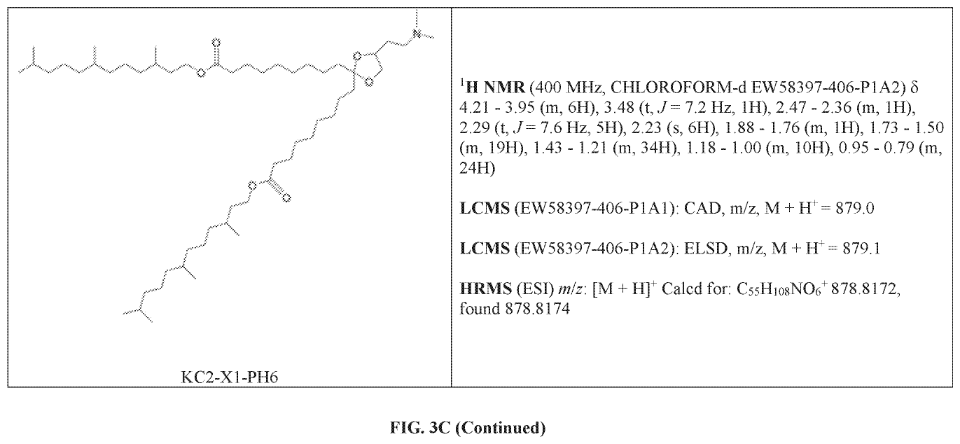

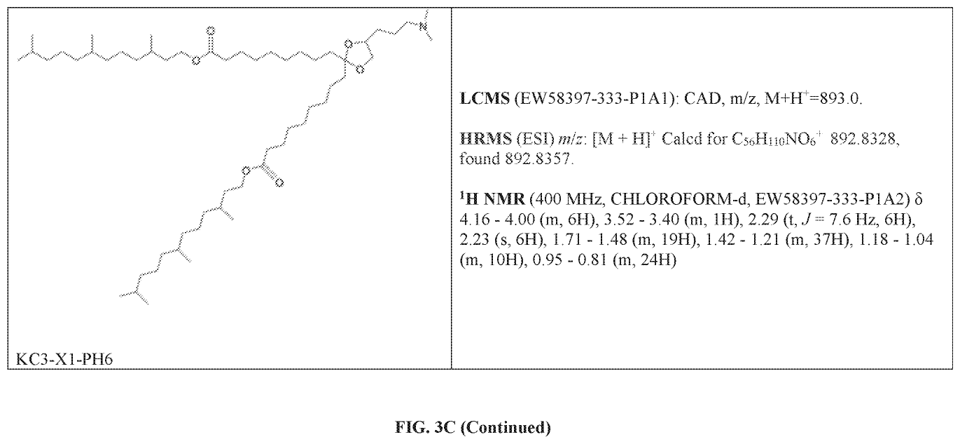

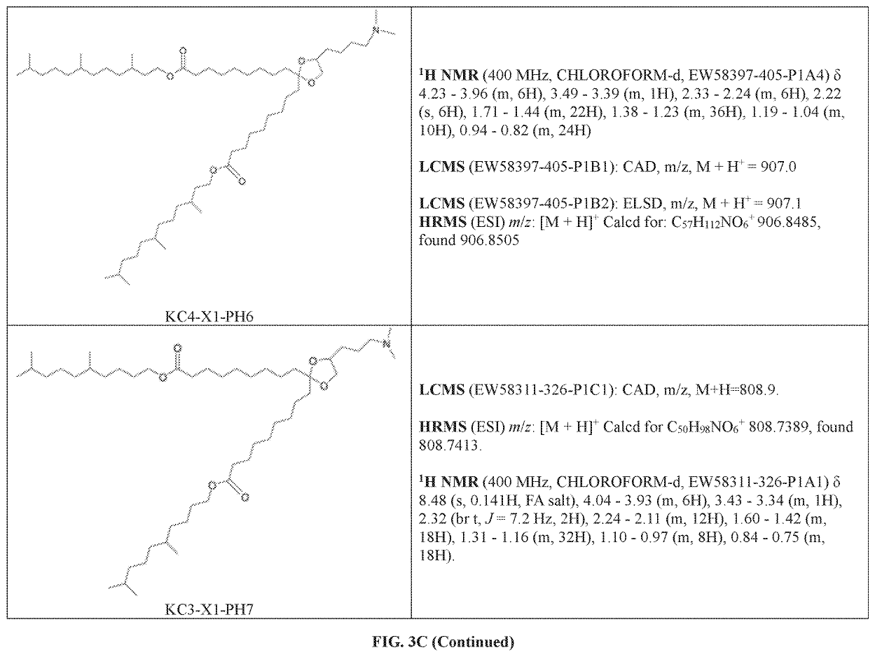

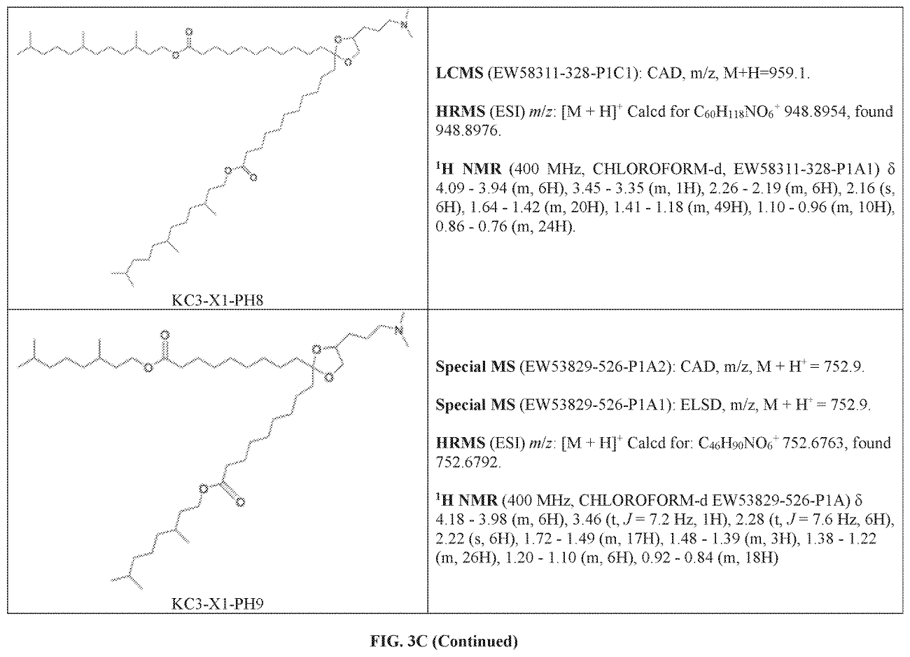

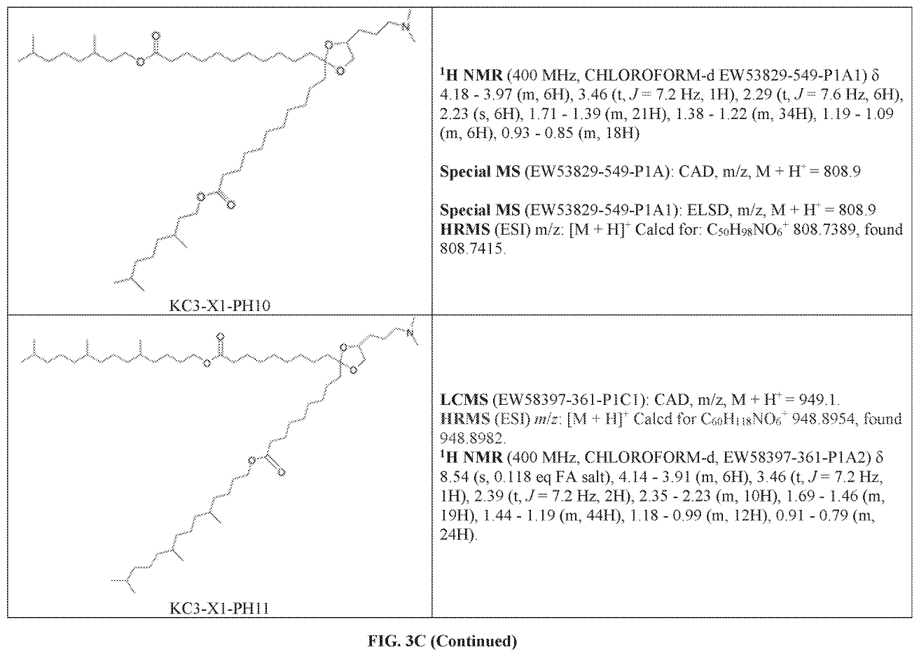

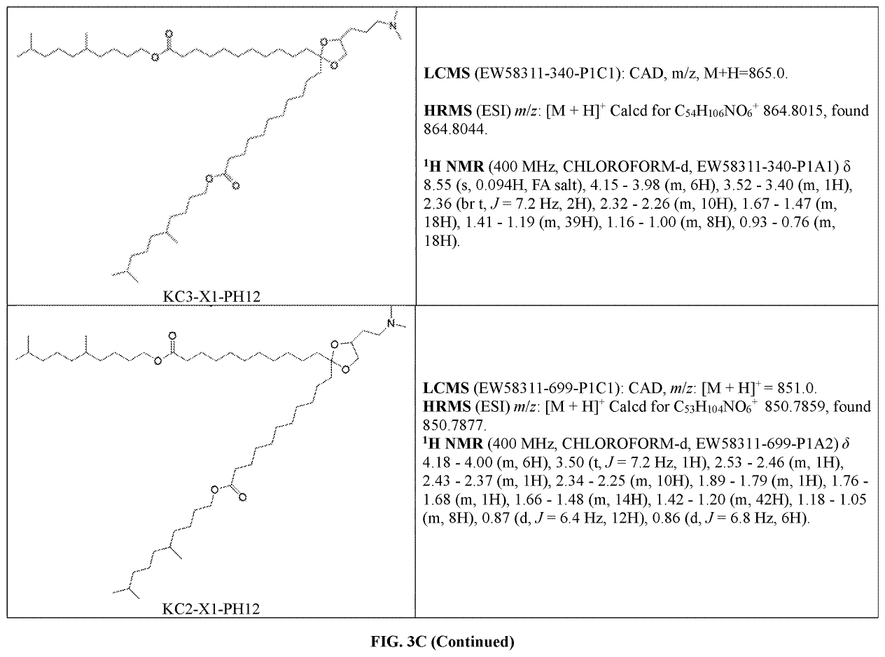

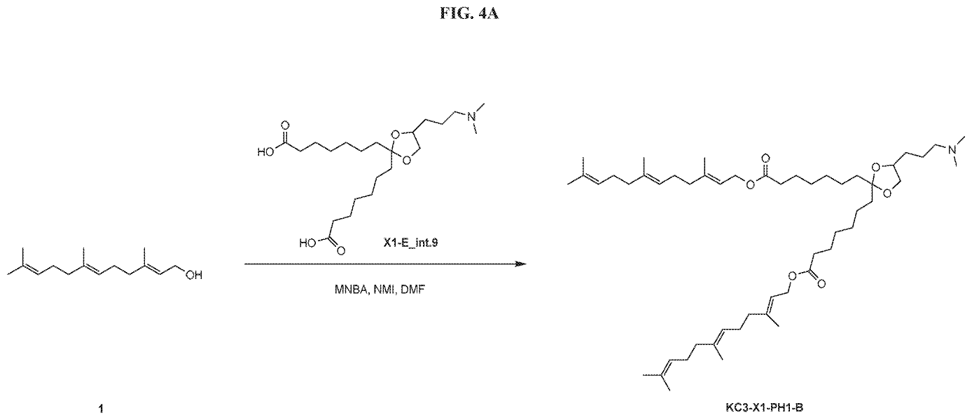

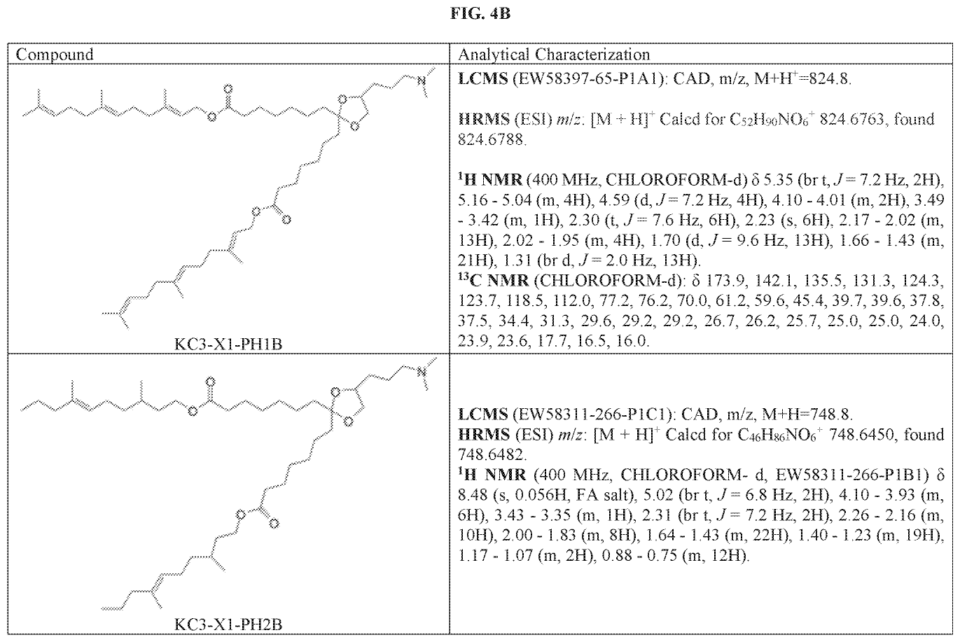

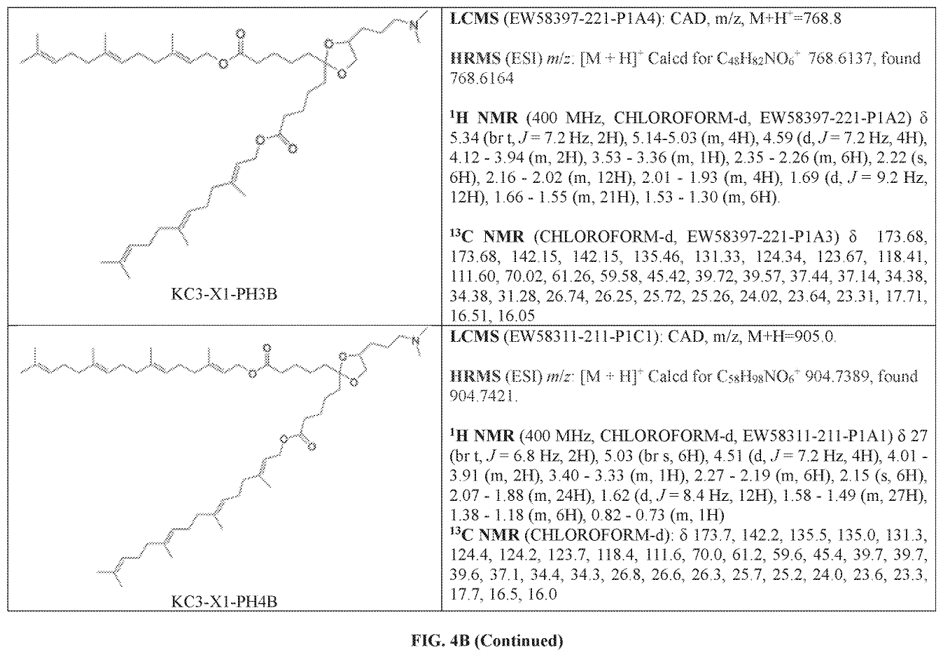

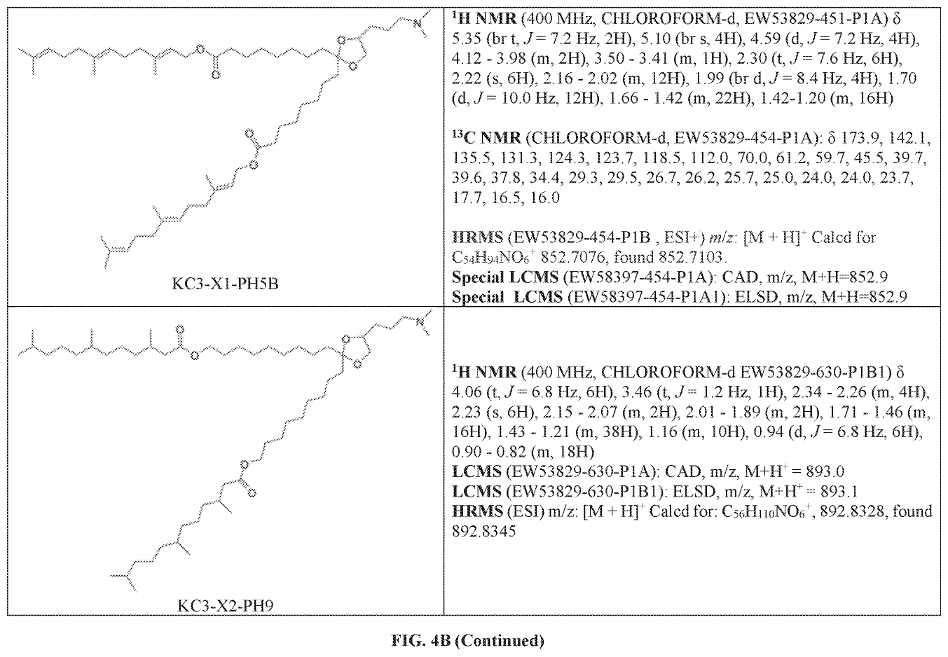

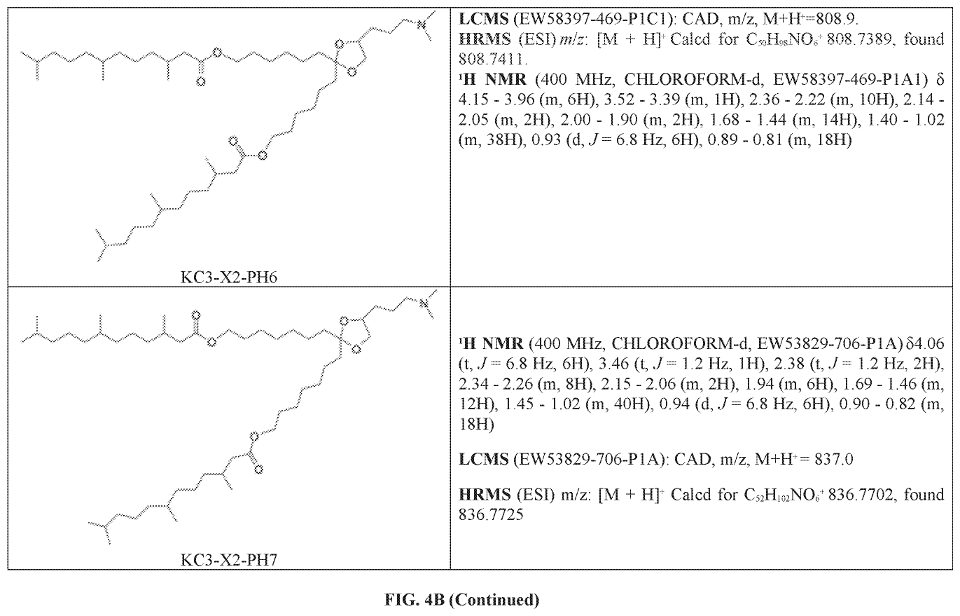

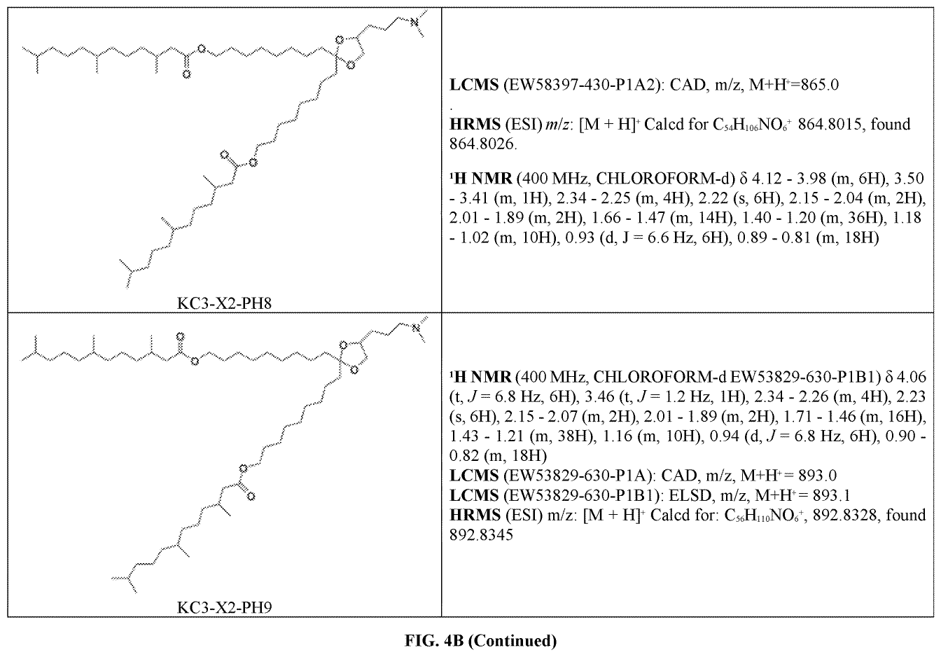

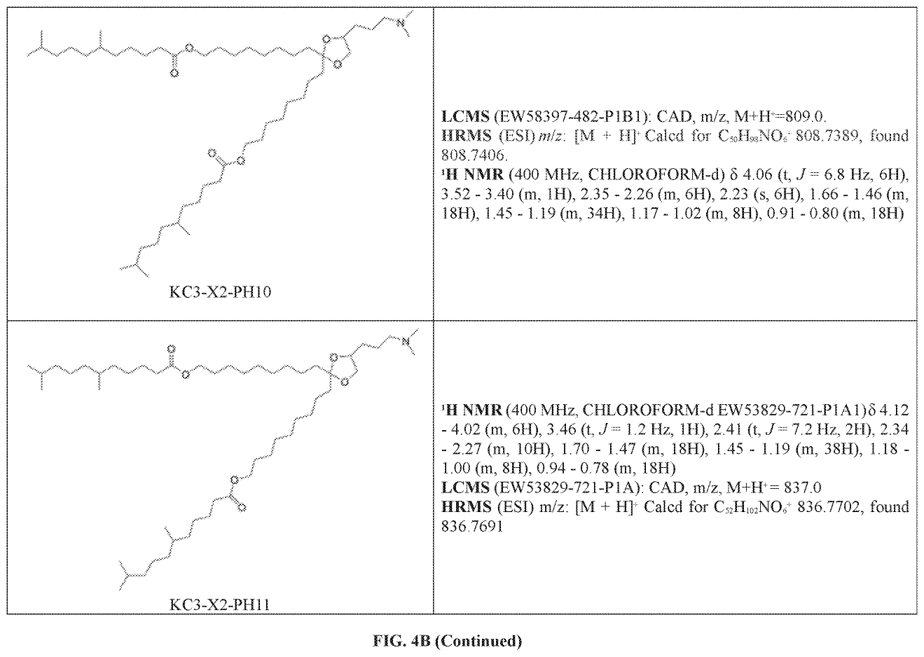

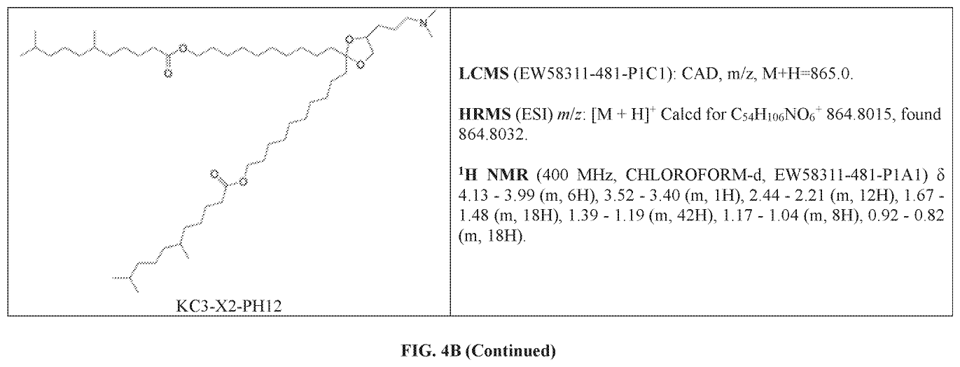

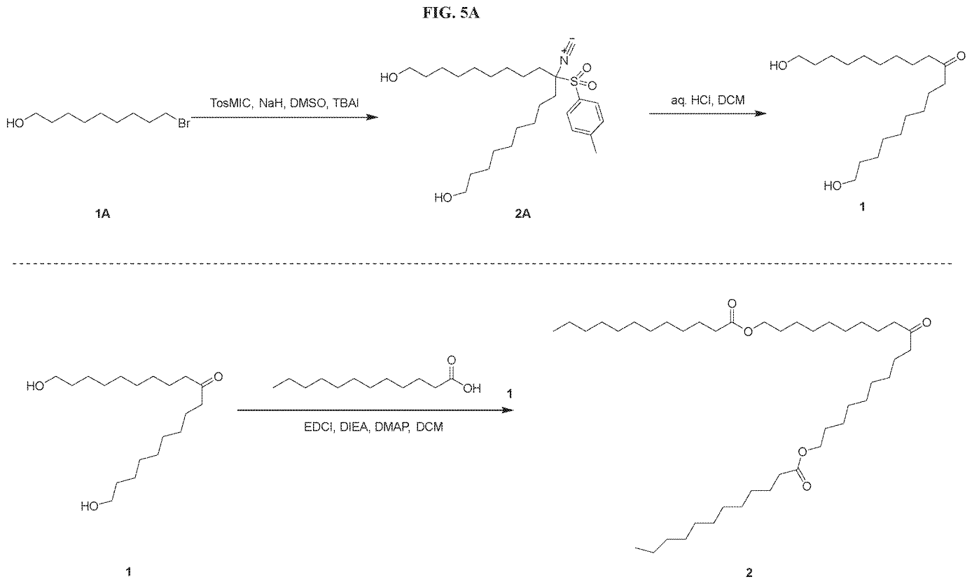

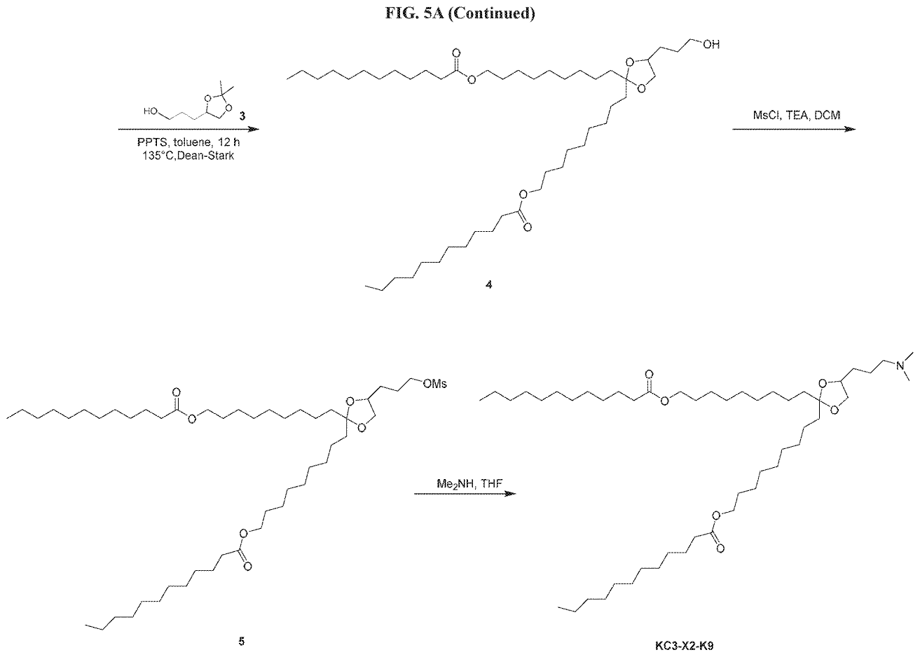

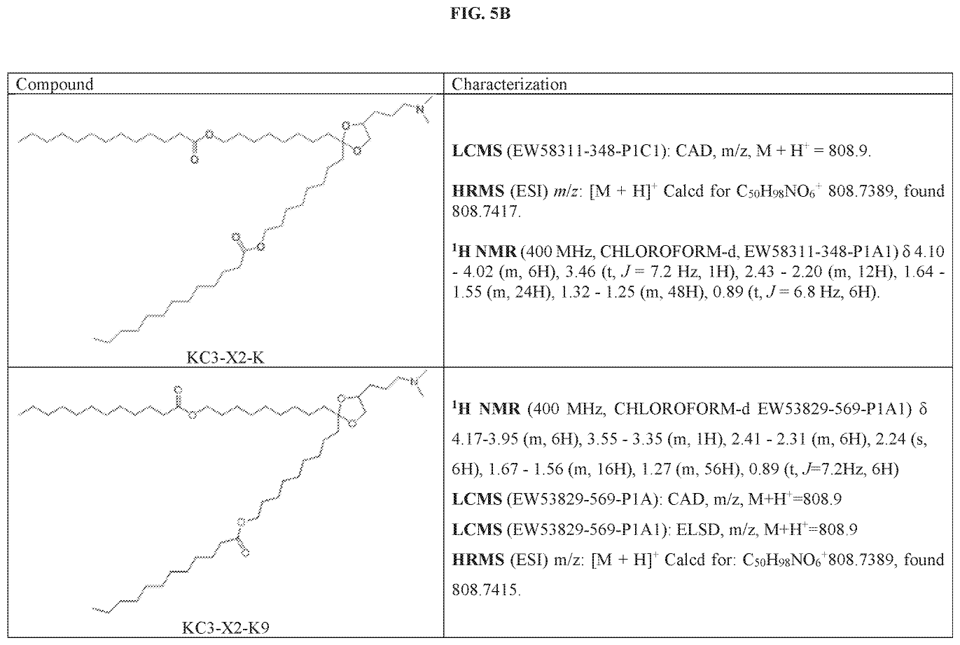

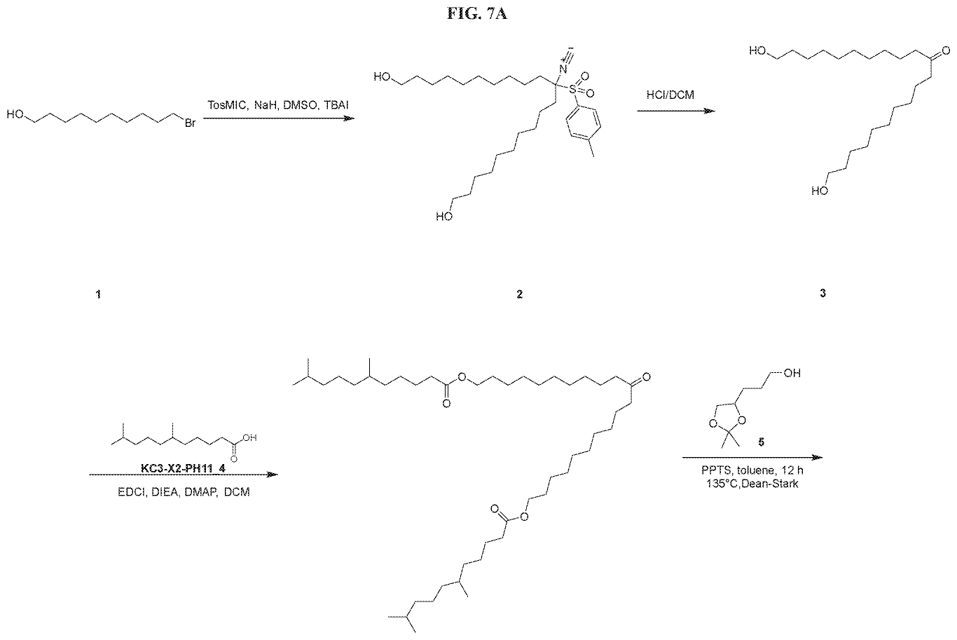

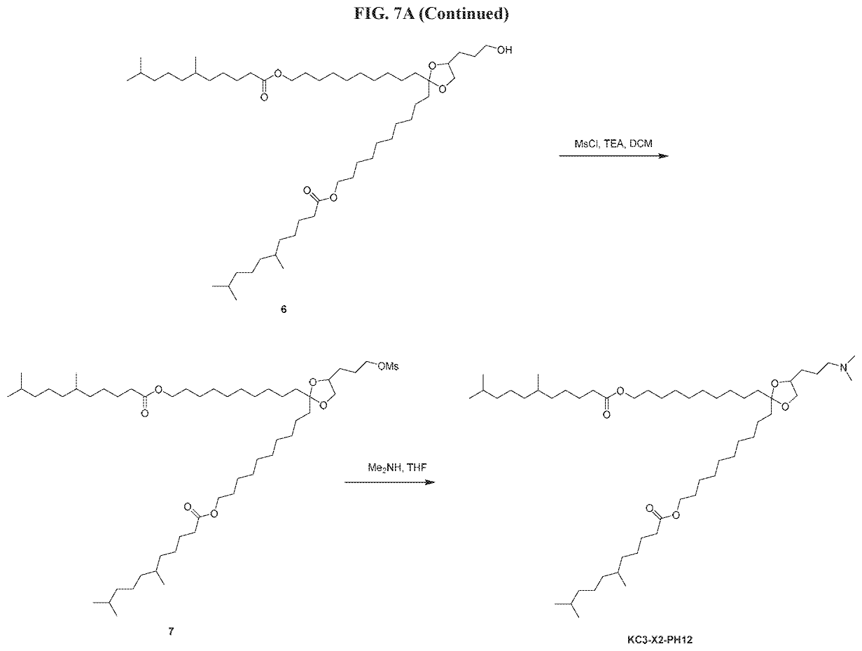

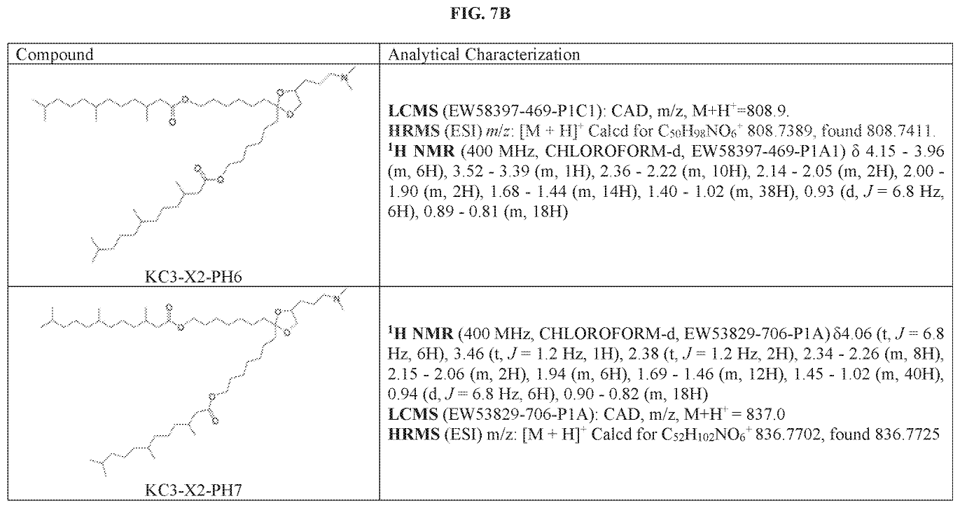

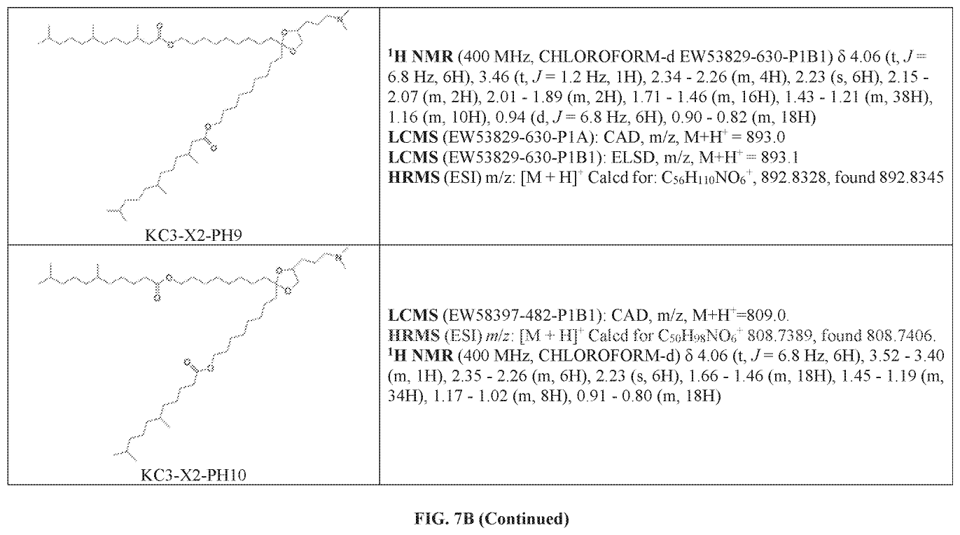

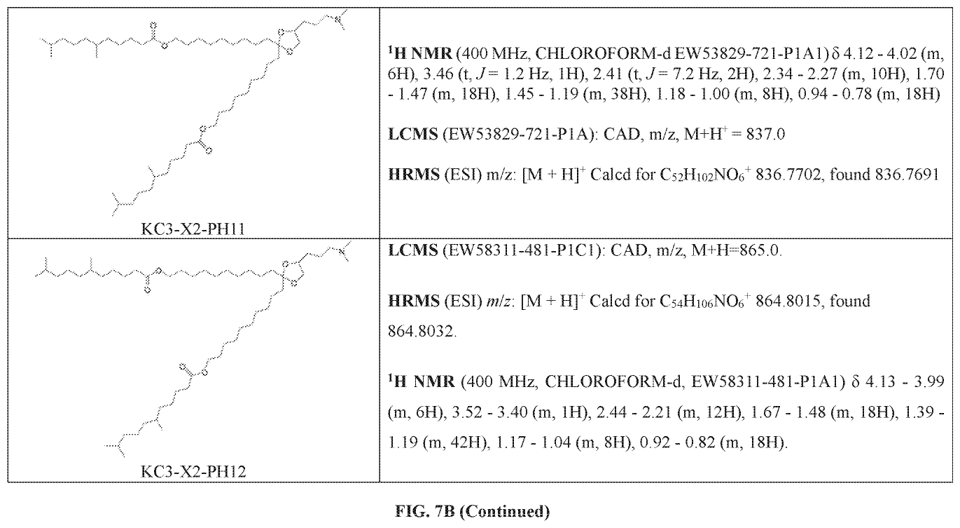

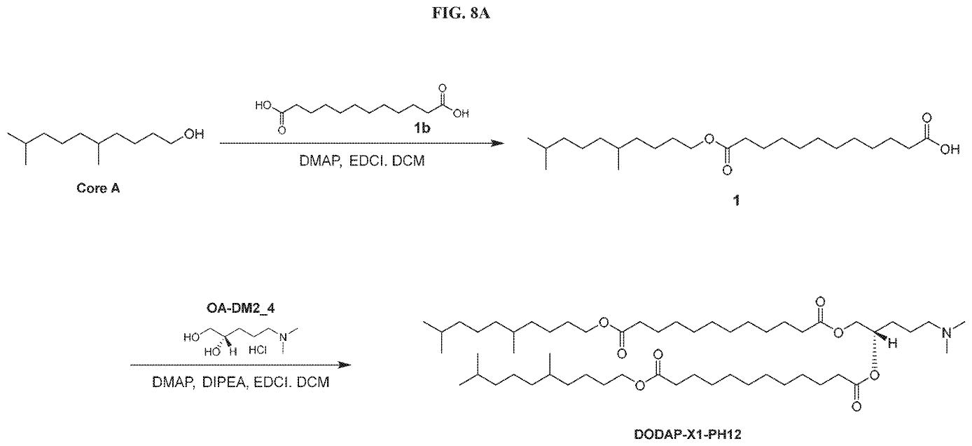

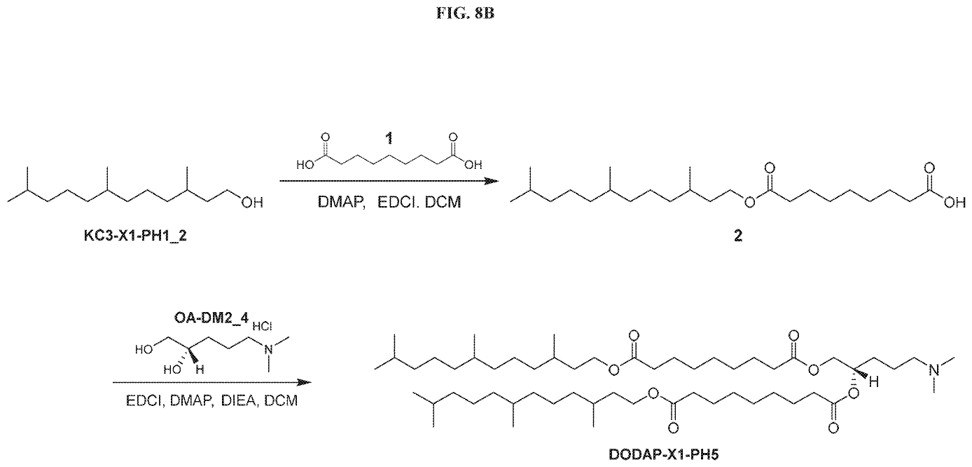

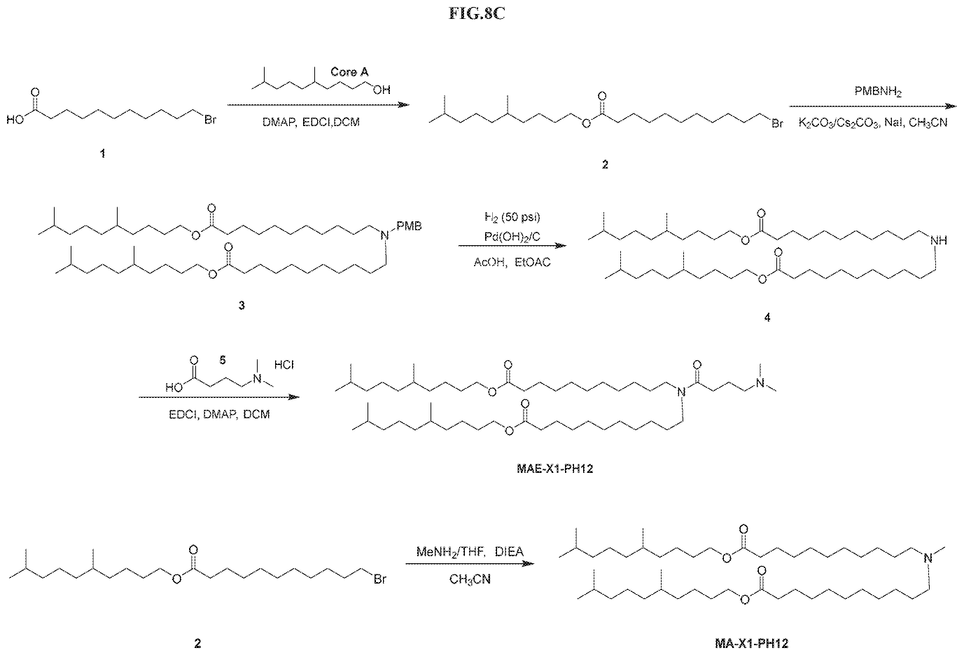

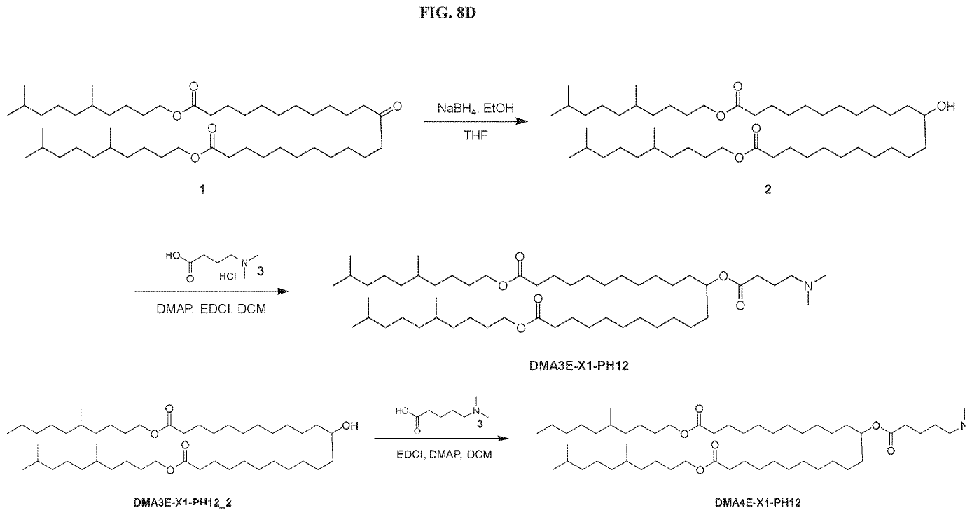

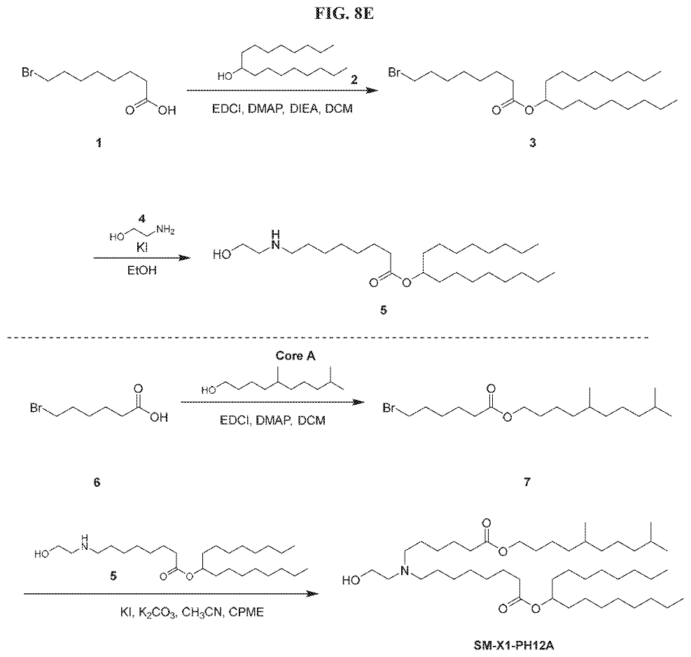

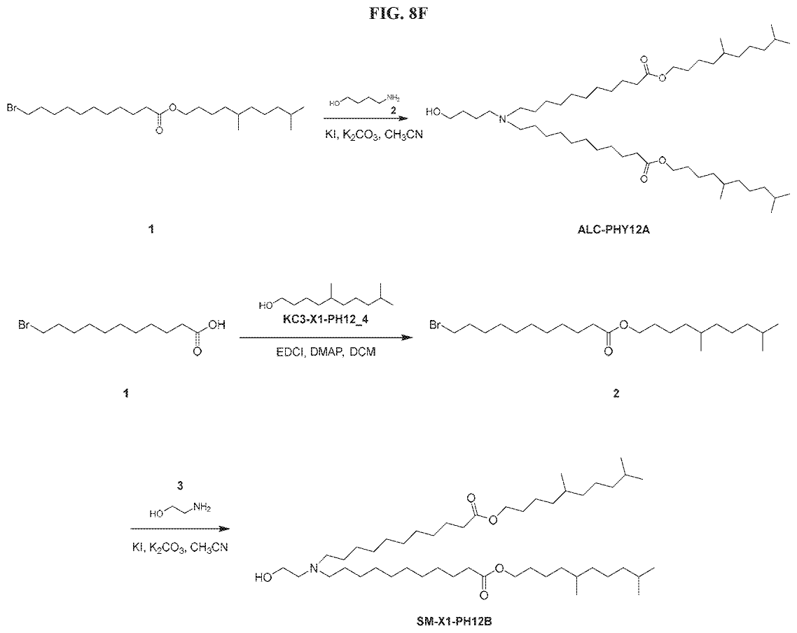

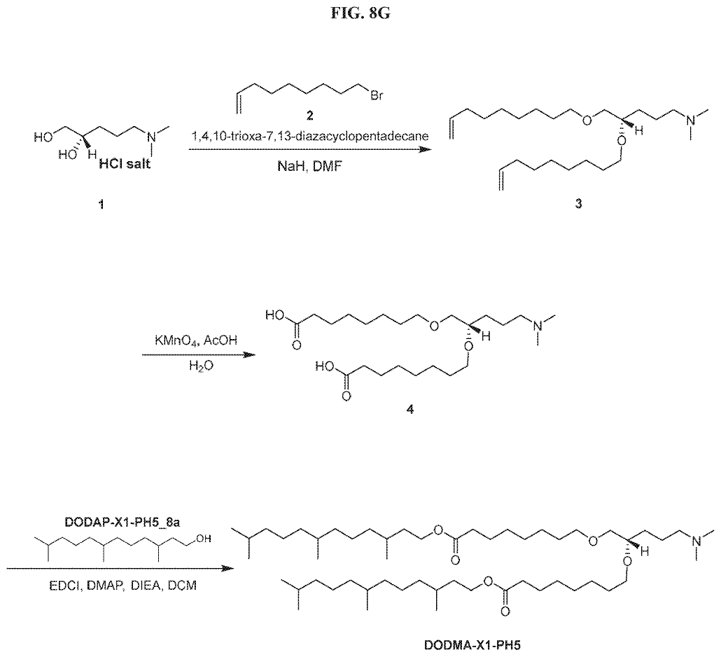

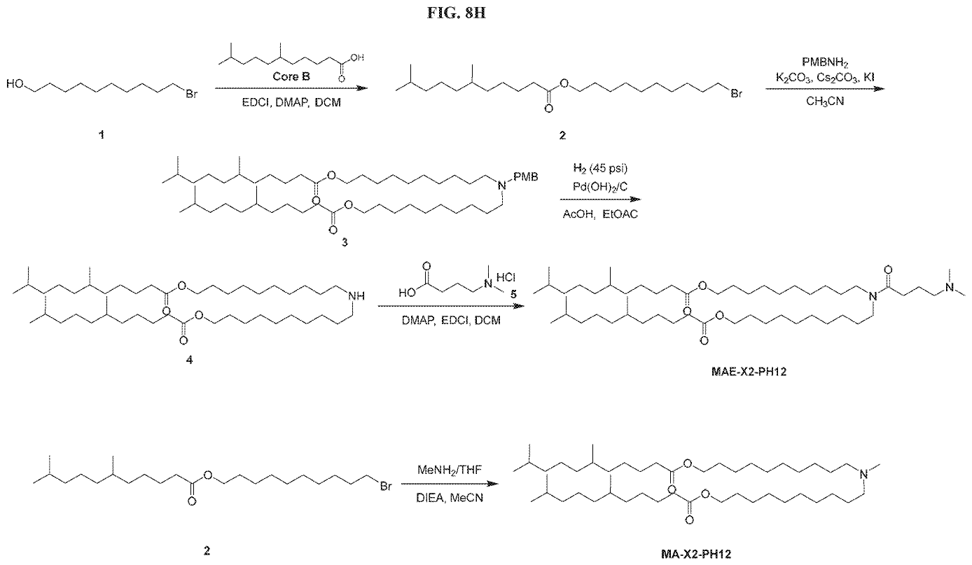

In some embodiments, an ionizable lipid compound comprises (a) an alkylamine head group having a pKa of 6-7.5, covalently bound to two or more linear or branched functionalized aliphatic chains each comprising one or more bioreducible or hydrolysable functional group(s) such as an ester, and two or more branched C₅ hydrocarbon monomer units such as isopentane (IPN) or 2-methylbut-2-ene (2 MB). Examples of such compounds include compounds in the tables in FIG. 3C, FIG. 4B, FIG. 5B, FIG. 7B and FIG. 8J.

In some embodiments, an ionizable lipid compound consists of a dioxolane or dioxane ring covalently bound to both (a) an alkylamine head group having a pKa of 6-7.5, and (b) a pair of identical geminal functionalized aliphatic chains covalently bound to the dioxolane or dioxane ring, each having (x+y) total carbon atoms and each comprising: (i) a linear aliphatic group having a total of (x) carbon atoms covalently bound to the dioxolane or dioxane ring and to (ii) an ester functional group, and (iii) a linear or branched aliphatic chain having a total of (y) carbon atoms and covalently bound to the ester functional group, each comprising one or more branched C₅ hydrocarbon monomer units, wherein (x) is 1-10, (y) is 6-20, and (x+y) is 16-25; or (x) is 7-10, (y) is 10-20, and (x+y) is 20-25; or (x) is 8-10, (y) is 10-20, and (x+y) is 21-25.

In some embodiments, an ionizable lipid compound consists of a dioxolane or dioxane ring covalently bound to both (a) an alkylamine head group having a pKa of 6-7.5, and (b) a pair of identical geminal functionalized aliphatic chains covalently bound to the dioxlane or dioxane ring, each having 18-25 total carbon atoms and each comprising: (i) a 7-10 carbon linear aliphatic group covalently bound to the dioxolane or dioxane ring and to (ii) an ester functional group, and (iii) a 10-18 carbon linear or branched aliphatic chain covalently bound to the ester functional group, optionally comprising one or more olefins and/or two or more branched C₅ hydrocarbon monomer units. In some embodiments, an ionizable lipid compound consists of a dioxolane or dioxane ring covalently bound to both (a) an alkylamine head group having a pKa of 6-7.5, and (b) a pair of identical geminal functionalized aliphatic chains covalently bound to the dioxlane or dioxane ring, each having 18-25 total carbon atoms and each comprising: (i) a 7-10 carbon linear aliphatic group covalently bound to the dioxolane or dioxane ring and to (ii) an ester functional group, and (iii) a 10-18 carbon linear or branched aliphatic chain covalently bound to the ester functional group, comprising two or more branched C₅ hydrocarbon monomer units, such as branched aliphatic chains having a total of two or three 2-methylbut-2-ene (2 MB) monomers. In some embodiments, an ionizable lipid compound consists of a 1,3-dioxolane ring covalently bound to both (a) an alkylamine head group having a pKa of 6-7.5, and (b) a pair of identical geminal functionalized aliphatic chains covalently bound to the 2-position of the 1,3-dioxolane ring, each having 20-25 total carbon atoms and each comprising: (i) a 7-10 carbon linear aliphatic group covalently bound to the 1,3-dioxolane ring and to (ii) an ester functional group, and (iii) a 10-18 carbon linear or branched aliphatic chain covalently bound to the ester functional group, optionally comprising one or more olefins and/or two or more branched C₅ hydrocarbon monomer units. In some embodiments, an ionizable lipid compound consists of a 1,3-dioxolane ring covalently bound to both (a) an alkylamine head group having a pKa of 6-7.5, and (b) a pair of identical geminal functionalized aliphatic chains covalently bound to the 2-position of the 1,3-dioxolane ring, each having 18-25 total carbon atoms and each comprising: (i) a 8-10 carbon linear aliphatic group covalently bound to the 1,3-dioxolane ring and to (ii) an ester functional group, and (iii) a 10-18 carbon linear or branched aliphatic chain covalently bound to the ester functional group, optionally comprising one or more olefins and/or two or more branched C₅ hydrocarbon monomer units, such as branched aliphatic chains having a total of two or three 2-methylbut-2-ene (2 MB) monomers. In some embodiments, an ionizable lipid compound consists of a 1,3-dioxolane ring covalently bound to both (a) an alkylamine head group having a pKa of 6-7.5, and (b) a pair of identical geminal functionalized aliphatic chains covalently bound to the 2-position of the 1,3-dioxolane ring, each having 21-25 total carbon atoms and each comprising: (i) a 7-10 carbon linear aliphatic group covalently bound to the 1,3-dioxolane ring and to (ii) an ester functional group, and (iii) a 10-18 carbon linear or branched aliphatic chain covalently bound to the ester functional group, optionally comprising one or more olefins and/or two or more branched C₅ hydrocarbon monomer units, such as branched aliphatic chains having a total of two or three 2-methylbut-2-ene (2 MB) monomers. In some embodiments, the dioxolane ring is a 1,3-dioxolane ring and each of the functionalized aliphatic chains comprises one or more olefins. In some embodiments, the dioxolane ring is a 1,3-dioxolane ring and each of the functionalized aliphatic chains comprises two or more branched C₅ hydrocarbon monomer units such as isopentane (IPN) or 2-methylbut-2-ene (2 MB).

In some embodiments, the present disclosure provides ionizable cationic lipid compounds of Formula (I):

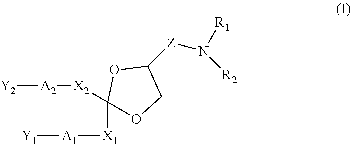

or a pharmaceutically acceptable salt thereof, wherein
R₁ and R₂ are each independently C₁₋₄ alkyl optionally substituted with hydroxyl;
Z is *—(CH₂)ₙ—**, wherein * is a covalent bond to the 1,3-dioxolane ring and ** is a covalent bond to —NR₁R₂, and n is 2, 3 or 4;
—X₁— and —X₂— are the same of different and are each independently —(CH₂)₍ₓ₎—, wherein (x) is 7-10;
-A₁- and -A₂- are the same or different and are each independently biodegradable or hydrolysable groups;
—Y₁— and —Y₂— are the same or different and are each an aliphatic chain having (y) total carbon atoms, optionally comprising two or more branched C₅ hydrocarbon monomer units, wherein (y) is 10-17; and
the sum of (x)+(y) is 18-25.

In some embodiments, ionizable lipid is a compound of Formula (I-A):

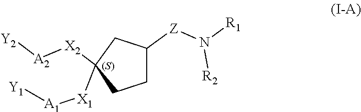

or a pharmaceutically acceptable salt thereof, wherein R₁, R₂, Z, X₁, X₂, A₁, A₂, Y₁ and Y₂ are as defined with respect to Formula (I).

In some embodiments, the alkylamine headgroup comprises an alkylamine that is substituted with one or two aliphatic groups, such as an ethyl(dialkylamino) group or a ethyl(alkylamino) group. In some embodiments, the head group comprises an alkylamino or dialkylamino group is a secondary or tertiary amine having a pKa of about 6-7.5 that comprises an amine substituted with one or two methyl or ethyl groups. In some embodiments, the head group comprises an alkylamino or dialkylamino group is a secondary or tertiary amine having a pKa of about 6-7.5 that comprises an amine substituted with one or two methyl or ethyl groups optionally substituted with a hydroxyl. In some embodiments, the alkylamine headgroup is —(CH₂)₂₋₄—N(Rₙ₁)(Rₙ₂), wherein Rₙ₁ is H, methyl or ethyl, and Rₙ₂ is H, methyl, ethyl, or —(CH₂)₂₋₄OH). In some embodiments, the alkylamine headgroup is —(CH₂)₂₋₄—N(CH₃)₂. In some embodiments, the alkylamine headgroup is —(CH₂)₂—N(CH₃)₂. In some embodiments, the alkylamine headgroup is —(CH₂)₃—N(CH₃)₂. In some embodiments, the alkylamine headgroup is —(CH₂)₄—N(CH₃)₂. In some embodiments, R₁ and R₂ in compounds of Formula (I) and Formula (I-A) are each methyl and n is 2, 3 or 4. In some embodiments, n is 2 and R₁ and R₂ are each methyl. In some embodiments, n is 3 and R₁ and R₂ are each methyl. In some embodiments, n is 4 and R₁ and R₂ are each methyl. In some embodiments, *—Z—N(R₁)(R₂) is selected from —(CH₂)₂₋₄—N(CH₃)₂, and —(CH₂)₂₋₄—N(CH₃)—(CH₂)₂₋₄—OH, wherein * is a covalent bond to the 1,3-dioxolane ring in Formula (I).

In some embodiments, the compound is a compound of Formula (I), wherein R₁ and R₂ are each methyl and n is 2, 3 or 4. In some embodiments, the compound is a compound of Formula (I), wherein n is 2 and R₁ and R₂ are each methyl. In some embodiments, the compound is a compound of Formula (I), wherein n is 3 and R₁ and R₂ are each methyl. In some embodiments, the compound is a compound of Formula (I), wherein n is 4 and R₁ and R₂ are each methyl. In some embodiments, the compound is a compound of Formula (I), wherein n is 2, 3 or 4, R₁ is —(CH₂)₂—OH and R₂ is methyl. In some embodiments, the compound is a compound of Formula (I), wherein n is 2, R₁ is —(CH₂)₂—OH and R₂ is methyl. In some embodiments, the compound is a compound of Formula (I), wherein n is 3, R₁ is —(CH₂)₂—OH and R₂ is methyl. In some embodiments, the compound is a compound of Formula (I), wherein n is 4, R₁ is —(CH₂)₂—OH and R₂ is methyl. In some embodiments, the compound is a compound of Formula (I), wherein R₁ and R₂ are each C₁₋₄ alkyl optionally substituted with hydroxyl. In some embodiments, the compound is a compound of Formula (I), wherein R₁ is C₁₋₄ alkyl and R₂ is C₁₋₄ alkyl optionally substituted with hydroxyl. In some embodiments, the compound is a compound of Formula (I), wherein R₁ is methyl and R₂ is —(CH₂)₂₋₄—OH. In some embodiments, the compound is a compound of Formula (I), wherein R₁ is methyl and R₂ is —(CH₂)₂—OH. In some embodiments, the compound is a compound of Formula (I), wherein R₁ is methyl and R₂ is —(CH₂)₃—OH. In some embodiments, the compound is a compound of Formula (I), wherein R₁ is methyl and R₂ is —(CH₂)₄—OH. In some embodiments, —X₁— and —X₂— are both —(CH₂)₍ₓ₎—, wherein (x) is 7, 8, 9 or 10; A₁ and A₂ are the same and are both independently *—C(O)—O—** or *—O—C(O)—**, wherein * is a covalent bond to Y₁ and Y₂, respectively, and ** is a covalent bond to X₁ and X₂, respectively; and —Y₁— and —Y₂—are each an aliphatic chain having (y) total carbon atoms, comprising two or more branched C₅ hydrocarbon monomer units, wherein (y) is 12, 13, 14, or 15; and wherein the sum of (x)+(y) is 22. In some embodiments, —Y₁ and —Y₂ are each independently *—(CH₂)₀₋₂—(R_{y})₂₋₄—H wherein each R_{y} is independently an isopentane (IPN) or a 2-methylbut-2-ene (2 MB) hydrocarbon monomer unit, and * is a covalent bond to A₁ or A₂. In some embodiments, R_{y} is —CH₂—CH(—CH₃)—CH₂—CH₂- or —CH₂—C(—CH₃)═CH—CH₂—. In some embodiments, the ionizable lipid is a compound of Formula (I), wherein X₁-A₁-Y₁ and —X₂-A₂-Y₂ are identical.

In some embodiments, the compound is a compound of Formula (I), wherein —X₁— and —X₂— are each independently —(CH₂)₍ₓ₎—, wherein (x) is 7, 8, 9 or 10; -A₁- and -A₂- are the same or different and are each independently hydrolysable or bioreducible groups; —Y₁— and —Y₂— are the same or different and are each an aliphatic chain comprising two or more branched C₅ hydrocarbon monomer units and having (y) total carbon atoms, wherein (y) is 10, 11, 12, 13, 14, 15, 16 or 17; and the sum of (x)+(y) is 18-25.

In some embodiments, an ionizable lipid compound comprises one or more biodegradable or hydrolysable functional group(s) such as an ester (e.g., carboxyl ester or carbonate ester), a carbamate, an amide or an ether. Examples of hydrolysable functional groups that can be included in the functionalized aliphatic tails of ionizable cationic lipids disclosed herein—such as the functional groups at A₁ and A₂ in Formula (I) and Formula (IA)—include carboxyl ester groups in any orientation (e.g., —C(═O)—O— or —O—C(═O)—), and other groups that can be degraded by esterases in vivo and allow tuning of degradation rates through structural modification. Additional examples of hydrolysable groups that can be incorporated in the functionalized aliphatic tails of the ionizable cationic lipids—such as A₁ and A₂ in Formula (I) and Formula (IA)—include: carbonate esters (—O—CO—O—) which can be more labile than simple esters and enable faster breakdown, or carbamates in any orientation (—NH—CO—O—) to provide intermediate stability between esters and amides. In addition, the hydrolysable functional aliphatic tails of the ionizable cationic lipids can include amide bonds in either orientation (—CONH— or —NHCO—) which can be more resistant to hydrolysis, but can still be cleaved enzymatically and can be used to provide greater structural stability. In addition, the functionalized aliphatic tails of the ionizable cationic lipids—including the functional groups at A₁ and A₂ in Formula (I) and Formula (IA)—can be acid-sensitive groups such as ketals, acetals, and orthoesters incorporated to enable pH-triggered degradation within the acidic environment of endosomes, thereby promoting mRNA release.

In some embodiments, the present disclosure provides ionizable cationic lipid compounds of Formula (I) or Formula (I-A), wherein -A₁- and -A₂- are the same or different and are each independently carboxyl ester or carbonate ester groups in any orientation. In some embodiments, the present disclosure provides ionizable cationic lipid compounds of Formula (I) or Formula (I-A), wherein -A₁- and -A₂- are the same or different and are each independently a carbamate group in any orientation. In some embodiments, the present disclosure provides ionizable cationic lipid compounds of Formula (I) or Formula (I-A), wherein -A₁- and -A₂- are the same or different and are each independently amide groups in any orientation. In some embodiments, the present disclosure provides ionizable cationic lipid compounds of Formula (I) or Formula (I-A), wherein -A₁- and -A₂- are the same or different and are each independently ether groups. In some embodiments, the compound is a compound of Formula (I), wherein each of A₁ and A₂ is a hydrolysable group such as an ester, carbonate, carbamate, acetal, ketal, orthoester, acid-labile dioxolane, phosphoester or phosphoramidate. In some embodiments, an ionizable lipid compound comprises a total of two linear or branched functionalized aliphatic chains, each comprising (i) a saturated linear aliphatic group (X) covalently bound to an alkylamine headgroup and to (ii) a biodegradable or hydrolysable functional group, and (iii) a linear or branched aliphatic chain (Y) optionally comprising one or more alkene groups, covalently bound to the bioreducible or hydrolysable functional group.

In some embodiments, the compound is a compound of Formula (I), wherein —Y₁ and —Y₂ comprise one or more branched C₅ hydrocarbon monomer units, such as n-pentane, isopentane, neopentane, 1-pentene, cis-2-pentene, trans-2-pentene, 3-methyl-1-butene, 2-methylbut-2-ene, 2-methylbut-1-ene, 3-methylbut-1-ene (isopentane), isoprene, 1,4-pentadiene, 1,5-pentadiene, 1,3-pentadiene (cis, trans or cis/trans), 2-methyl-1,3-pentadiene, 3-methyl-1,3-pentadiene, 1-pentyne, 2-pentyne, 3-methyl-1-butyne, or 3-methyl-2-butyne (isopentyne). In some embodiments, the compound is a compound of Formula (I), wherein —Y₁ and —Y₂ comprise two, three or four C₅ hydrocarbon monomer units in a configuration forming a covalent bond from the C₁ carbon of a C₅ hydrocarbon monomer to the C₄ carbon of an adjacent C₅ hydrocarbon monomer, such as IPN (—CH₂—CH(—CH₃)—CH₂—CH₂—) and/or 2 MB (—CH₂—C(—CH₃)═CH—CH₂—). In some embodiments, the compound is a compound of Formula (I), wherein —X₁-A₁-Y₁ and —X₂-A₂-Y₂ are identical; —X₁— and —X₂— are each independently —(CH₂)₍ₓ₎—, wherein (x) is 7, 8, 9 or 10; each of A₁ and A₂ is independently *—C(O)—O—** or *—O—C(O)—**, wherein * is a covalent bond to Y₁ and Y₂, respectively, and ** is a covalent bond to X₁ and X₂, respectively; —Y₁ and —Y₂ are each *—(CH₂)₀₋₂—(R_{y})₂₋₄—(CH₂)₀₋₂—H wherein each R_{y} is independently a C₅ hydrocarbon monomer unit such as an IPN or a 2 MB hydrocarbon monomer unit, and * is a covalent bond to A₁ or A₂; (y) is 12, 13, 14 or 15; and the sum of (x)+(y) is 22.

Some aspects of the disclosure relate to bioreducible ionizable cationic lipids of Formula (I) or (I-A), wherein each R₁ is independently linear hydrocarbon alkyl chains each comprising a hydrolysable or bioreducible group, such as an ester group (e.g, —O—CO— or —CO—O—); and R₂ and R₃ are each independently (C₁-C₄) alkyl optionally substituted with hydroxyl; and n is an integer equal to 2, 3 or 4.

In some embodiments, a bioreducible or hydrolysable ionizable cationic lipid comprises a compound of Formula (I) or Formula (I-A) wherein each of R₂ and R₃ is methyl, —Z— is —(CH₂)ₙ- and n is 3 or 4 and each R₁ is the same. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one or two olefin groups. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising one olefin group. In some embodiments, a bioreducible or hydrolysable ionizable cationic lipid comprises a compound of Formula (I) or Formula (I-A) wherein each of R₂ and R₃ is methyl and each R₁ is a hydrocarbon chain comprising a single ester group with a total 15-22 atoms, and optionally comprising two olefin groups separated by at least two methylene groups.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same or different and each comprises an ester joining a proximal saturated C₇₋₈ hydrocarbon at —X₁— and —X₂— and a distal C₁₂₋₁₅ hydrocarbon at —Y₁ and —Y₂ each optionally comprising one or more olefins and one or more methyl substituents.

In some embodiments, a bioreducible or hydrolysable ionizable cationic lipid comprises a compound of Formula (I) or Formula (I-A) wherein each of R₁ and R₂ is methyl, Z is —(CH₂)ₙ— and n is 3 or 4 and each of —X₁-A₁-Y₁ and —X₂-A₂-Y₂ is the same or different and each comprises an ester at -A₁- and -A₂- joining a proximal saturated C₄-10 hydrocarbon at —X₁— and —X₂— and a distal C10-20 hydrocarbon at —Y₁ and —Y₂ optionally comprising one or more olefins and one or more methyl substituents.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—,
n is 3 or 4 and each of —X₁-A₁-Y₁ and —X₂-A₂-Y₂ is the same and each comprises a proximal C₇₋₁₀ hydrocarbon at —X₁— and —X₂— and a distal saturated isoprenoid alkane group at —Y₁ and -Y₂ joined by an ester at -A₁- and -A₂-. In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same and each comprises a proximal C₇₋₁₀ hydrocarbon at —X₁— and —X₂— and a distal saturated phytane group at —Y₁ and —Y₂ joined by an ester at -A₁- and -A₂-. In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 or 4 and each of —X₁-A₁-Y₁ and —X₂-A₂-Y₂ is the same and each comprises a proximal C₇₋₁₀ hydrocarbon at —X₁— and —X₂— and a distal unsaturated phytane group at —Y₁ and —Y₂ comprising one or more olefins joined by an ester at -A₁- and -A₂-.

In some embodiments, a bioreducible or hydrolysable ionizable cationic lipid comprises a compound of Formula (I) or Formula (I-A) wherein each of R₁ and R₂ is methyl, —Z— is —(CH₂)ₙ—, n is 3 and each of —X₁-A₁-Y₁ and —X₂-A₂-Y₂ is the same and each comprises a proximal C₇₋₁₀ hydrocarbon at —X₁— and —X₂— joined by an ester at -A₁- and -A₂- to a distal branched hydrocarbon chain comprising two or three isoprene or isopentane monomer groups at —Y₁ and —Y₂. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 or 4 and each —X₁-A₁-Y₁ and —X₂-A₂-Y₂ are each —(CH₂)₇₋₁₀-A-Y, wherein A is —C(═O)—O— or —O—C(═O)—, and Y is —(CH₂—CH₂—CH(—CH₃)—CH₂)₃—H or —(CH₂—CH₂—CH(—CH₃)—CH₂)₃—H or —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 or 4 and each —X₁-A₁-Y₁ and —X₂-A₂-Y₂ is —(CH₂)₇-A-Y, wherein A is —C(═O)—O— or —O—C(═O)—, and Y is —(CH₂—CH₂—CH(—CH₃)—CH₂)₃—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 or 4 and each —X₁-A₁-Y₁ and —X₂-A₂-Y₂ is —(CH₂)₈-A-Y, wherein A is —C(═O)—O— or —O—C(═O)—, and Y is —(CH₂—CH₂—CH(—CH₃)—CH₂)₃—H or —(CH₂)₂—CH═CH—(CH₂)₈—H or —(CH₂)₁₂—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 or 4 and each —X₁-A₁-Y₁ and —X₂-A₂-Y₂ is —(CH₂)₁₀-A-Y, wherein A is —C(═O)—O— or —O—C(═O)—, and Y is —CH₂—CH₂—(CH₂—CH₂—CH(—CH₃)—CH₂)₂—H.

In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 and each —X₁-A₁-Y₁ and —X₂-A₂-Y₂ are each —(CH₂)₇₋₁₀-A-Y, wherein A is —C(═O)—O— or —O—C(═O)—, and Y is —(CH₂—CH₂—CH(—CH₃)—CH₂)₃—H or —(CH₂—CH₂—CH(—CH₃)—CH₂)₃—H or —CH₂—CH₂—(CH₂—CH₂—CH(—CH₃)—CH₂)₂—H. In some embodiments, a bioreducible or hydrolysable ionizable cationic lipid comprises a compound of Formula (I) or Formula (I-A) wherein each of R₁ and R₂ is methyl, Z— is —(CH₂)ₙ—, n is 3 and each —X₁-A₁-Y₁ and —X₂-A₂-Y₂ is —(CH₂)₇-A-Y, wherein A is —C(═O)—O— or —O—C(═O)—, and Y is —(CH₂—CH₂—CH(—CH₃)—CH₂)₃—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 and each —X₁-A₁-Y₁ and —X₂-A₂-Y₂ is —(CH₂)₈-A-Y, wherein A is —C(═O)—O— or —O—C(═O)—, and Y is —(CH₂—CH₂—CH(—CH₃)—CH₂)₃—H or —(CH₂)₂—CH═CH—(CH₂)₈—H or —(CH₂)₁₂—H. In some embodiments, a bioreducible or hydrolysable ionizable cationic lipid comprises a compound of Formula (I) or Formula (I-A) wherein each of R₂ and R₃ is methyl, Z— is —(CH₂)ₙ—, n is 3 and each —X₁-A₁-Y₁ and —X₂-A₂-Y₂ is —(CH₂)₁₀-A-Y, wherein A is —C(═O)—O— or —O—C(═O)—, and Y is —CH₂—CH₂—(CH₂—CH₂—CH(—CH₃)—CH₂)₂—H.

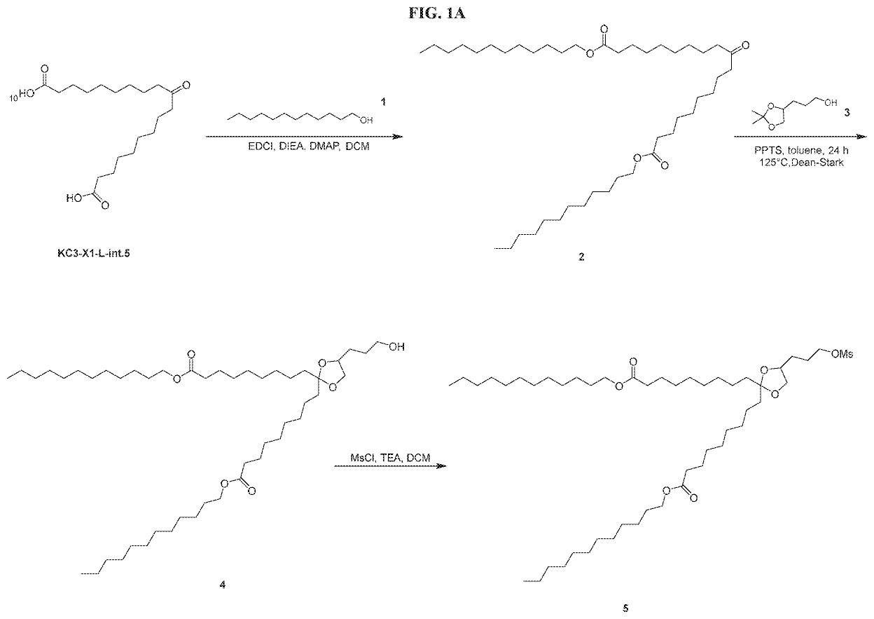

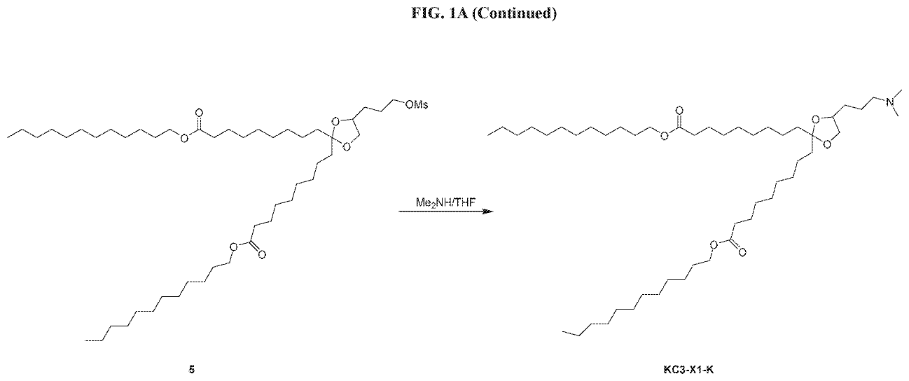

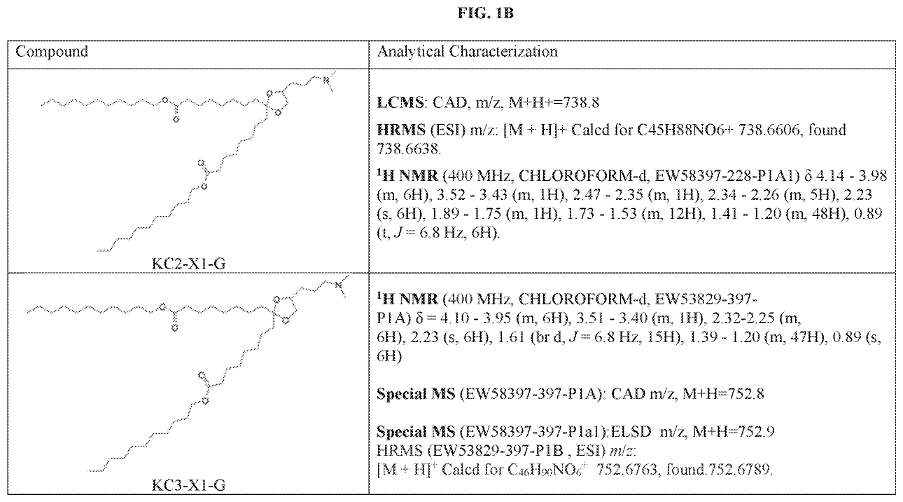

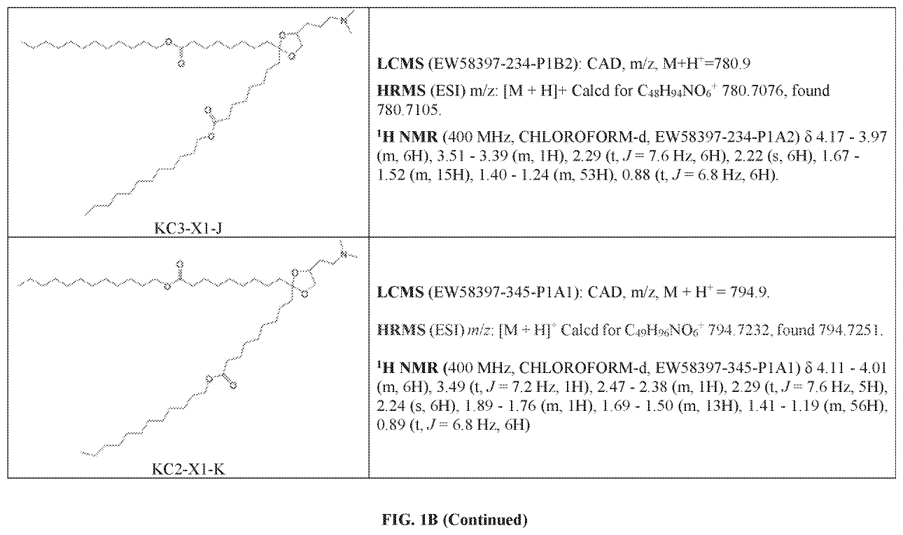

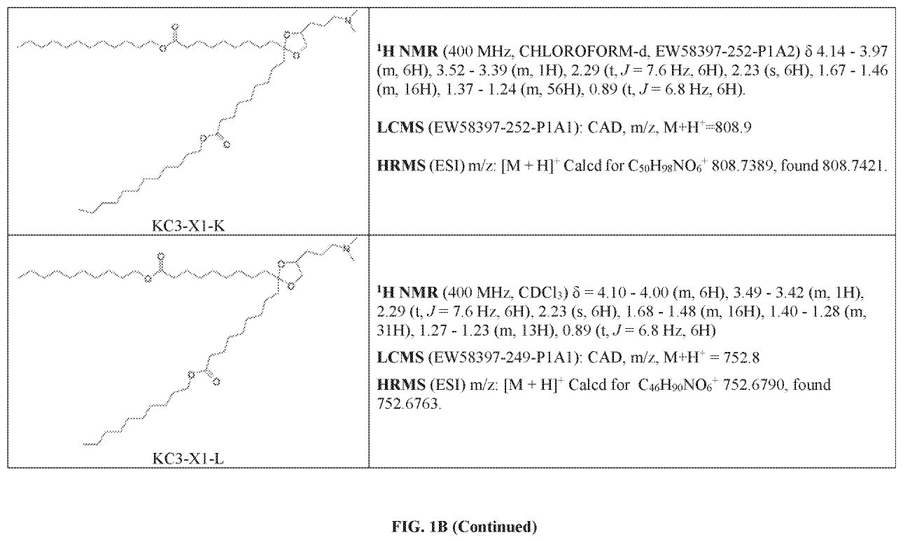

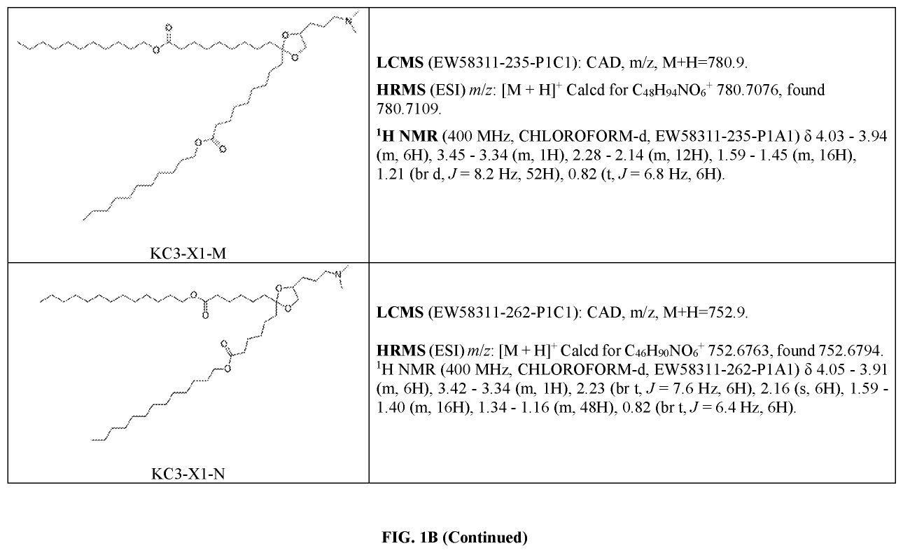

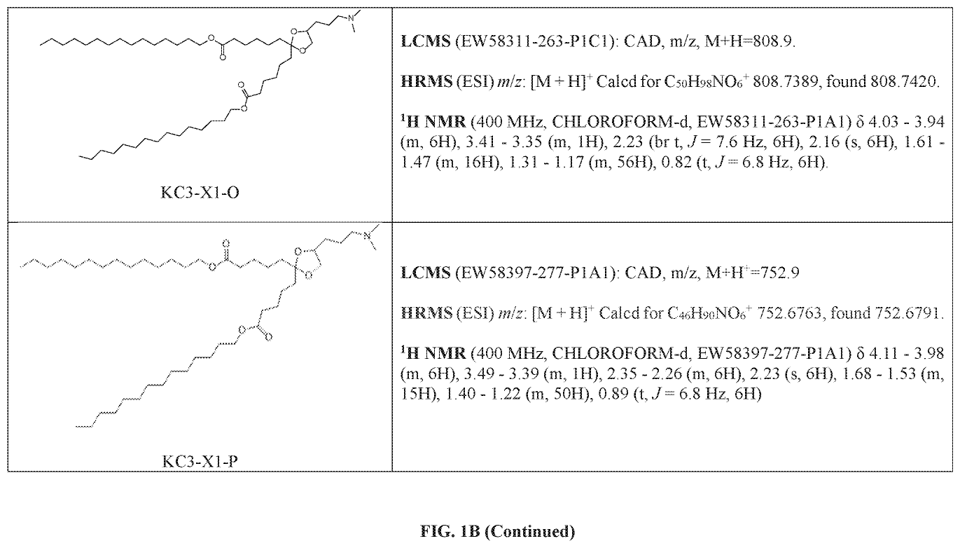

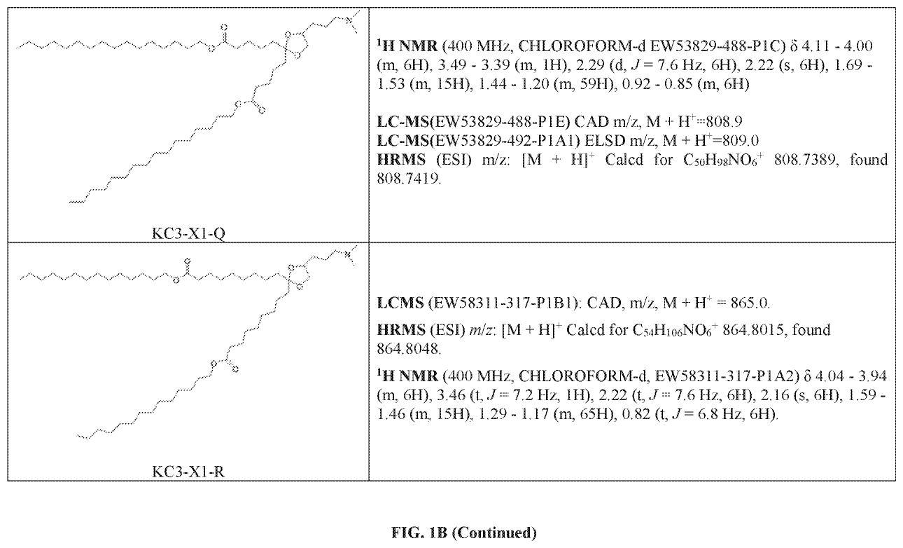

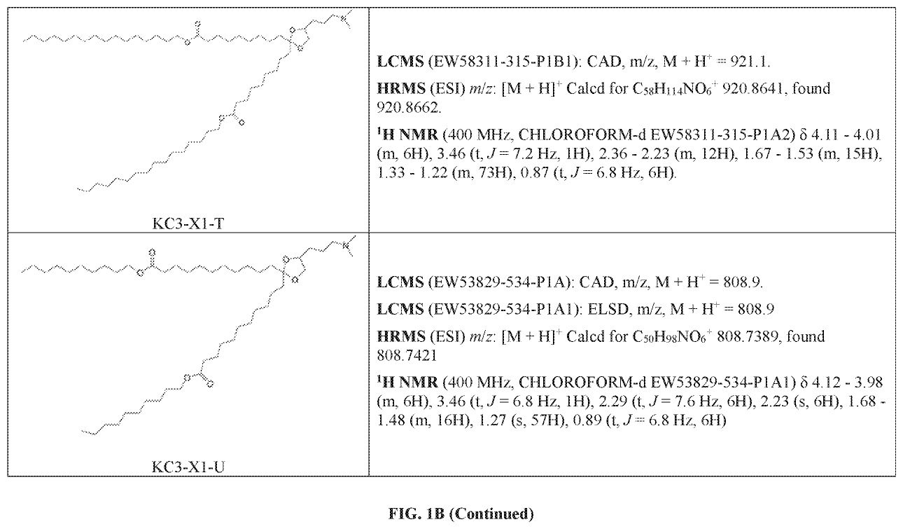

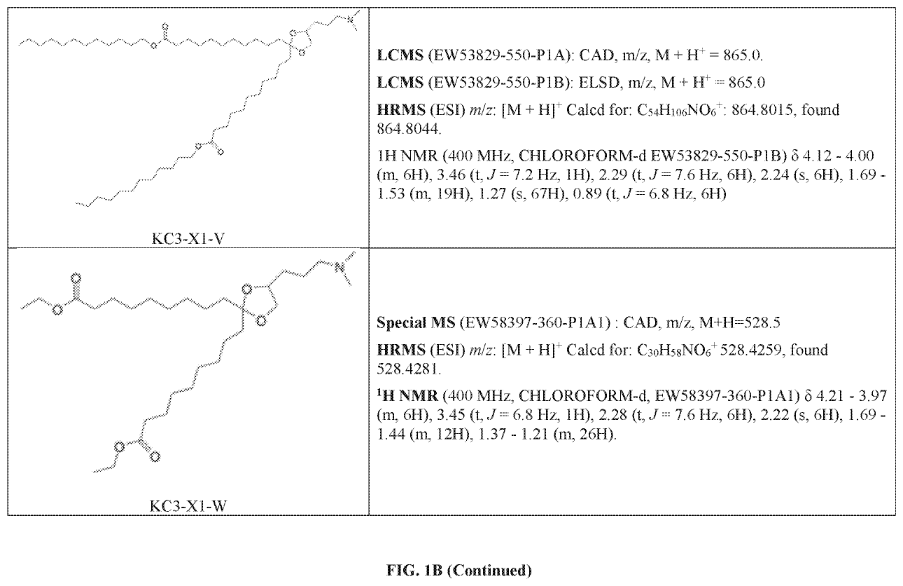

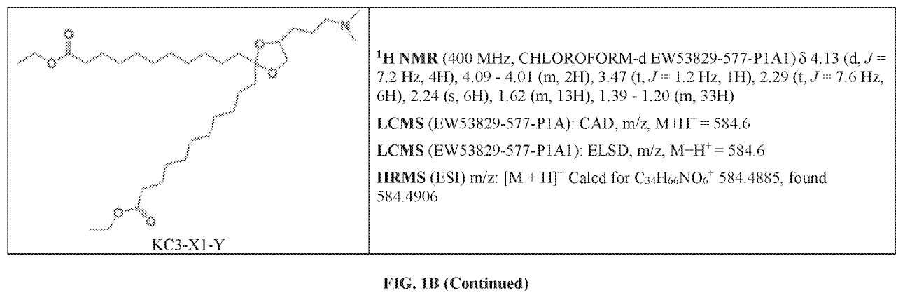

In some embodiments, the ionizable cationic lipid is a compound of Formula (I) or Formula (I-A) as described in the tables in FIG. 1B, 2B, 3C, 4B, 5B, 6B, or 7B.

In some embodiments, the ionizable cationic lipid is a compound of Formula (I) or Formula (I-A) as described in Table A.

TABLE A

| Compound | Y₁₋, Y₂₋ | (y) | A1, A2 | (x) | (x) + (y) |
|---|---|---|---|---|---|
| KCn-X1-PH1 | IPN-IPN-IPN- | 15 | *—O—C(O)—** | 6 | 21 |
| KCnX2-PH1 | IPN-IPN-IPN- | 15 | *—C(O)—O—** | 6 | 21 |
| KCn-X1-PH1B | 2MB-2MB-2MB- | 15 | *—O—C(O)—** | 6 | 21 |
| KCn-X2-PH1B | 2MB-2MB-2MB- | 15 | *—C(O)—O—** | 6 | 21 |
| KCn-X1-PH2 | IPN-IPN-(CH₂)₂— | 12 | *—O—C(O)—** | 6 | 18 |
| KCn-X2-PH2 | IPN-IPN-(CH₂)₂— | 12 | *—C(O)—O—** | 6 | 18 |
| KCn-X1-PH2B | 2MB-2MB-(CH₂)₂— | 12 | *—O—C(O)—** | 6 | 18 |
| KCn-X2-PH2B | 2MB-2MB-(CH₂)₂— | 12 | *—C(O)—O—** | 6 | 18 |
| KCn-X1-PH3 | IPN-IPN-IPN- | 15 | *—O—C(O)—** | 4 | 19 |
| KCn-X2-PH3 | IPN-IPN-IPN- | 15 | *—C(O)—O—** | 4 | 19 |
| KCn-X1-PH3B | 2MB-2MB-2MB- | 15 | *—O—C(O)—** | 4 | 19 |
| KCn-X2-PH3B | 2MB-2MB-2MB- | 15 | *—C(O)—O—** | 4 | 19 |
| KCn-X1-PH4 | IPN-IPN-IPN-IPN- | 20 | *—O—C(O)—** | 4 | 24 |
| KCn-X2-PH4 | IPN-IPN-IPN-IPN- | 20 | *—C(O)—O—** | 4 | 24 |
| KCn-X1-PH4B | 2MB-2MB-2MB-2MB- | 20 | *—O—C(O)—** | 4 | 24 |
| KCn-X2-PH4B | 2MB-2MB-2MB-2MB- | 20 | *—C(O)—O—** | 4 | 24 |
| KCn-X1-PH5 | IPN-IPN-IPN- | 15 | *—O—C(O)—** | 7 | 22 |
| KCn-X2-PH5 | IPN-IPN-IPN- | 15 | *—C(O)—O—** | 7 | 22 |
| KCn-X1-PH5B | 2MB-2MB-2MB- | 15 | *—O—C(O)—** | 7 | 22 |
| KCn-X1-PH5B | 2MB-2MB-2MB- | 15 | *—C(O)—O—** | 7 | 22 |
| KCn-X1-PH6 | IPN-IPN-IPN- | 15 | *—O—C(O)—** | 8 | 23 |
| KCn-X2-PH6 | IPN-IPN-IPN- | 15 | *—C(O)—O—** | 8 | 23 |
| KCn-X1-PH6B | 2MB-2MB-2MB- | 15 | *—O—C(O)—** | 8 | 23 |
| KCn-X2-PH6B | 2MB-2MB-2MB- | 15 | *—C(O)—O—** | 8 | 23 |
| KCn-X1-PH7 | IPN-IPN-(CH₂)₂— | 12 | *—O—C(O)—** | 8 | 20 |
| KCn-X2-PH7 | IPN-IPN-(CH₂)₂— | 12 | *—C(O)—O—** | 8 | 20 |
| KCn-X1-PH7B | 2MB-2MB-(CH₂)₂— | 12 | *—O—C(O)—** | 8 | 20 |
| KCn-X2-PH7B | 2MB-2MB-(CH₂)₂— | 12 | *—C(O)—O—** | 8 | 20 |
| KCn-X1-PH8 | IPN-IPN-IPN- | 15 | *—O—C(O)—** | 10 | 25 |
| KCn-X2-PH8 | IPN-IPN-IPN- | 15 | *—C(O)—O—** | 10 | 25 |
| KCn-X1-PH8 | 2MB-2MB-2MB- | 15 | *—O—C(O)—** | 10 | 25 |
| KCn-X2-PH8 | 2MB-2MB-2MB- | 15 | *—C(O)—O—** | 10 | 25 |
| KCn-X1-PH9 | IPN-IPN- | 10 | *—O—C(O)—** | 8 | 18 |
| KCn-X2-PH9 | IPN-IPN- | 10 | *—C(O)—O—** | 8 | 18 |
| KCn-X1-PH9B | 2MB-2MB- | 10 | *—O—C(O)—** | 8 | 18 |
| KCn-X2-PH9B | 2MB-2MB- | 10 | *—C(O)—O—** | 8 | 18 |
| KCn-X1-PH10 | IPN-IPN- | 10 | *—O—C(O)—** | 10 | 20 |
| KCn-X2-PH10 | IPN-IPN- | 10 | *—C(O)—O—** | 10 | 20 |
| KCn-X1-PH10 | 2MB-2MB- | 10 | *—O—C(O)—** | 10 | 20 |
| KCn-X2-PH10 | 2MB-2MB- | 10 | *—C(O)—O—** | 10 | 20 |
| KCn-X1-PH11 | IPN-IPN-IPN-(CH₂)₂— | 17 | *—O—C(O)—** | 8 | 25 |
| KCn-X2-PH11 | IPN-IPN-IPN-(CH₂)₂— | 17 | *—C(O)—O—** | 8 | 25 |
| KCn-X1-PH11B | 2MB-2MB-2MB-(CH₂)₂— | 17 | *—O—C(O)—** | 8 | 25 |
| KCn-X2-PH11B | 2MB-2MB-2MB-(CH₂)₂— | 17 | *—C(O)—O—** | 8 | 25 |
| KCn-X1-PH12 | IPN-IPN-(CH₂)₂— | 12 | *—O—C(O)—** | 10 | 22 |
| KCn-X2-PH12 | IPN-IPN-(CH₂)₂— | 12 | *—C(O)—O—** | 10 | 22 |
| KCn-X1-PH12B | 2MB-2MB-(CH₂)₂— | 12 | *—O—C(O)—** | 10 | 22 |
| KCn-X2-PH12B | 2MB-2MB-(CH₂)₂— | 12 | *—C(O)—O—** | 10 | 22 |
| KCn-X1-PH13 | IPN-IPN-2MB- | 15 | *—O—C(O)—** | 10 | 25 |
| KCn-X1-PH14 | IPN-2MB-IPN- | 15 | *—O—C(O)—** | 10 | 25 |
| KCn-X1-PH15 | 2MB-IPN-IPN- | 15 | *—O—C(O)—** | 10 | 25 |
| KCn-X1-PH16 | IPN-2MB-2MB- | 15 | *—O—C(O)—** | 10 | 25 |
| KCn-X1-PH17 | IPN-2MB-(CH₂)₂— | 12 | *—O—C(O)—** | 10 | 22 |
| KCn-X1-PH18 | 2MB-IPN-(CH₂)₂— | 12 | *—O—C(O)—** | 10 | 22 |
| KCn-X1-PH19 | CH₃—CH₂-IPN-IPN- | 12 | *—O—C(O)—** | 10 | 22 |
| KCn-X1-PH20 | CH₃—CH₂-2MB-IPN- | 12 | *—O—C(O)—** | 10 | 22 |

In some embodiments, the ionizable lipid is a compound of Formula (I) wherein (n) can be 2, 3 or 4 (KCn), X₁ and X₂ are the same and are each —(CH₂)₍ₓ₎— with (x) is 7-10, A₁ and A₂ are the same and are an ester as shown in Table B (wherein * is a covalent bond to Y₁ or Y₂, respectively, and ** is a covalent bond to X₁ or X₂, respectively), and —Y₁— and —Y₂— are the same and are each *—(CH₂)₂—CH═CH—(CH₂)_{(y-4)}H with (y) is 10-20 wherein * in —Y₁— and —Y₂— is a covalent bond to A₁ or A₂, respectively and (x)+(y) is 18-25. In some embodiments, —Y₁— and —Y₂-in Formula (I) are the same or different and are each independently an aliphatic chain having (y) total carbon atoms, and comprising one or more olefins, wherein (y) is 10, 11, 12, 13, 14, 15, 16 or 17; and wherein the sum of (x)+(y) is 20, 21, 22, 23, 24 or 25. In some embodiments, —Y₁— and —Y₂— in Formula (I) are the same or different and are each independently an aliphatic chain having (y) total carbon atoms, and comprising a single olefin, wherein (x) is 7, 8, 9, or 10, (y) is 11, 12, 13, 14, or 15, and wherein the sum of (x)+(y) is 20, 21, 22, 23, 24 or 25. In some embodiments, —Y₁— and —Y₂— in Formula (I) are the same or different and are each independently an aliphatic chain having (y) total carbon atoms, and comprising a two olefins, wherein (x) is 7, 8, 9, or 10, (y) is 11, 12, 13, 14, or 15, and wherein the sum of (x)+(y) is 20, 21, 22, 23, 24 or 25. In some embodiments, —Y₁— and —Y₂— in Formula (I) are the same or different and one or both of —Y₁— and —Y₂— are each independently *—(CH₂)₂—CH═CH—(CH₂)_{(y-4)}H, wherein * is a covalent bond to A₁ or A₂. In some embodiments, —Y₁— and —Y₂— in Formula (I) are the same or different and one or both of —Y₁— and —Y₂— are each independently an aliphatic chain comprising the moiety *—(CH₂)_{(c)}—CH═CH—(CH₂)₍₁₋₄₎—CH═CH—(CH₂)_{(d)}H, wherein (c)+(d)=3, 4, 5, 6, 7, 8, 9 or 10 and * is a covalent bond to A₁ or A₂. In some embodiments, —Y₁— and —Y₂— in Formula (I) are the same or different and one or both of —Y₁— and —Y₂— are each independently an aliphatic chain comprising the moiety *—(CH₂)_{(c)}—CH═CH—(CH₂)₄—CH═CH—(CH₂)_{(d)}H, wherein (c)+(d)=3, 4, 5, 6, or 7 and * is a covalent bond to A₁ or A₂. In some embodiments, —Y₁— and —Y₂— in Formula (I) are the same or different and one or both of —Y₁— and —Y₂— are each independently an aliphatic chain comprising the moiety *—(CH₂)_{(c)}—CH═CH—(CH₂)—CH═CH—(CH₂)_{(d)}H, wherein (c)+(d)=6, 7, 8, 9 or 10 and (y)=(c)+(d)+5 and * is a covalent bond to A₁ or A₂. In some embodiments, the ionizable lipid is a compound of Formula (I) wherein (n) can be 2, 3 or 4 (KCn), X₁ and X₂ are the same and are each —(CH₂)₍ₓ₎— with (x) being 8-10, A₁ and A₂ are the same and are an ester as shown in Table B (wherein * is a covalent bond to Y₁ or Y₂, respectively, and ** is a covalent bond to X₁ or X₂, respectively), and —Y₁— and —Y₂— are the same and are each *—(CH₂)₂—CH═CH—(CH₂)_{(y-4)}H with (y) is 10-20 and (x)+(y) is 18-24, wherein * in —Y₁— and —Y₂— is a covalent bond to A₁ or A₂, respectively.

TABLE B

| Compound | (y) | A1, A2 | (x) | (x) + (y) |
|---|---|---|---|---|
| KCn-X1-H | 11 | *—O—C(O)—** | 7 | 18 |
| KCn-X2-H | 11 | *—C(O)—O—** | 7 | 18 |
| KCn-X1-S | 12 | *—O—C(O)—** | 8 | 20 |
| KCn-X2-S | 12 | *—C(O)—O—** | 8 | 20 |
| KCn-X1-I | 13 | *—O—C(O)—** | 7 | 20 |
| KCn-X2-I | 13 | *—C(O)—O—** | 7 | 20 |
| KCn-X1-Z | 14 | *—O—C(O)—** | 8 | 19 |
| KCn-X2-Z | 14 | *—C(O)—O—** | 8 | 19 |
| KCn-X1-AA | 12 | *—O—C(O)—** | 10 | 22 |
| KCn-X2-AA | 12 | *—C(O)—O—** | 10 | 22 |

In some embodiments, the ionizable lipid is a compound of Formula (I), wherein —Y₁ and —Y₂ are the same or different and are each an aliphatic chain comprising a total of (y) carbon atoms. In some embodiments, the ionizable lipid is a compound of Formula (I), wherein —Y₁ and —Y₂ are the same or different and are each independently a linear or branched alkyl chain comprising a total of (y) carbon atoms. In some embodiments, the ionizable lipid is a compound of Formula (I), wherein —Y₁ and —Y₂ are the same or different and are each independently —(CH₂)_{(y)}H, wherein (y) is 11, 12, 13, 14 or 15. In some embodiments, the ionizable lipid is a compound of Formula (I), wherein —Y₁ and —Y₂ are the same and are each —(CH₂)_{(y)}H, wherein (y) is 12, 13, 14 or 15. In some embodiments, the ionizable lipid is a compound of Formula (I), wherein —Y₁ and —Y₂ are the same and are each —(CH₂)_{(y)}—, wherein (y) is 12, 13, 14 or 15. In some embodiments, the ionizable lipid is a compound of Formula (I), wherein —Y₁ and —Y₂ are the same and are each —(CH₂)_{(y)}H, wherein (x) is 7, 8, 9 or 10 and (y) is 12, 13, 14 or 15. In some embodiments, the ionizable lipid is a compound of Formula (I), wherein —Y₁ and —Y₂ are the same and are each —(CH₂)_{(y)}H, wherein (x) is 7 and (y) is 15 or wherein (x) is 10 and (y) is 12. In some embodiments, the ionizable lipid is a compound of Formula (I) wherein (n) can be 2, 3 or 4 (KCn), X₁ and X₂ are the same and are each —(CH₂)₍ₓ₎— with (x) as shown in Table C, A₁ and A₂ are the same and are an ester as shown in Table C (wherein * is a covalent bond to Y₁ or Y₂, respectively, and ** is a covalent bond to X₁ or X₂, respectively), and —Y₁— and —Y₂— are the same and are each *—(CH₂)_{(y)}—H with (b) as described in Table C, wherein * in —Y₁— and —Y₂— is a covalent bond to A₁ or A₂, respectively.

TABLE C

| Compound | (b) | A1, A2 | (a) | (a) + (b) |
|---|---|---|---|---|
| KCn-X1-G | 11 | *—O—C(O)—** | 7 | 18 |
| KCn-X2-G | 11 | *—C(O)—O—** | 7 | 18 |
| KCn-X1-K | 12 | *—O—C(O)—** | 8 | 20 |
| KCn-X2-K | 12 | *—C(O)—O—** | 8 | 20 |
| KCn-X1-L | 10 | *—O—C(O)—** | 8 | 18 |
| KCn-X2-L | 10 | *—C(O)—O—** | 8 | 18 |
| KCn-X1-M | 11 | *—O—C(O)—** | 8 | 19 |
| KCn-X2-M | 11 | *—C(O)—O—** | 8 | 19 |
| KCn-X1-R | 14 | *—O—C(O)—** | 8 | 22 |
| KCn-X2-R | 14 | *—C(O)—O—** | 8 | 22 |
| KCn-X1-T | 16 | *—O—C(O)—** | 8 | 24 |
| KCn-X2-T | 16 | *—C(O)—O—** | 8 | 24 |
| KCn-X1-U | 10 | *—O—C(O)—** | 10 | 20 |
| KCn-X2-U | 10 | *—C(O)—O—** | 10 | 20 |
| KCn-X1-V | 12 | *—O—C(O)—** | 10 | 22 |
| KCn-X2-V | 12 | *—C(O)—O—** | 10 | 22 |

In some embodiments, the ionizable lipid is selected from the group consisting of:

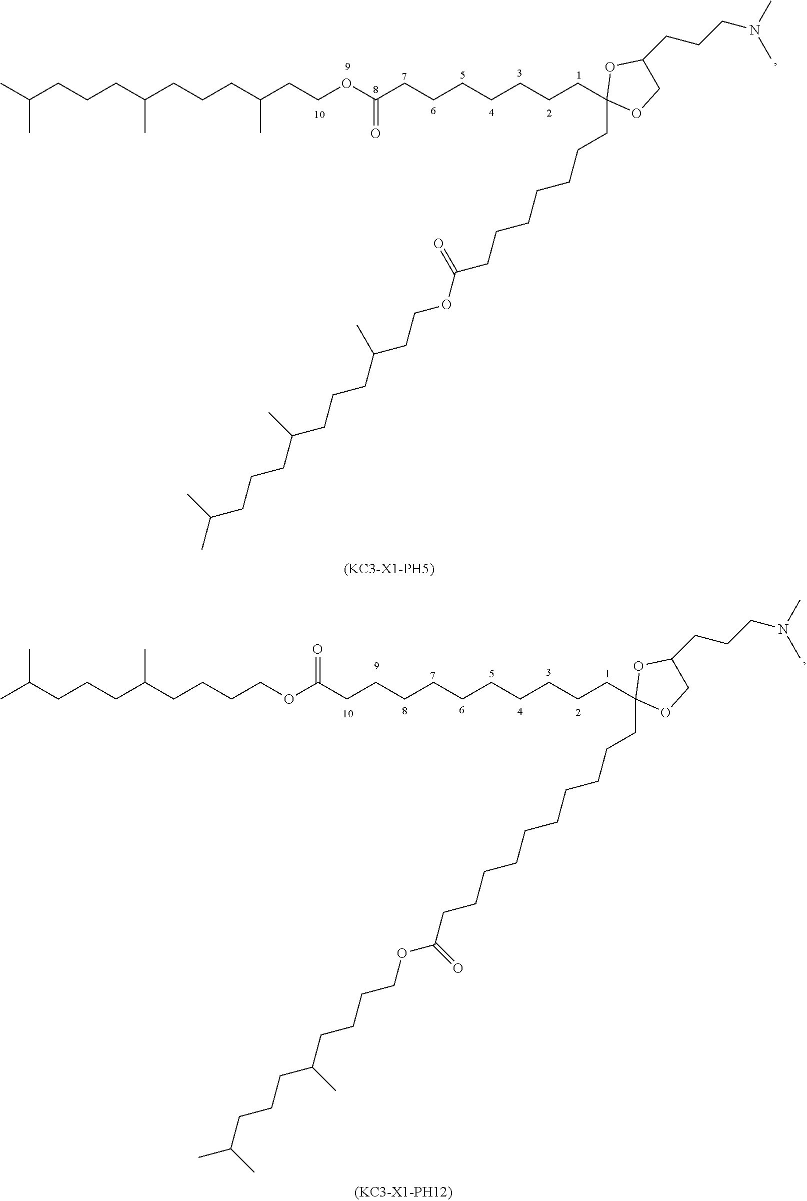

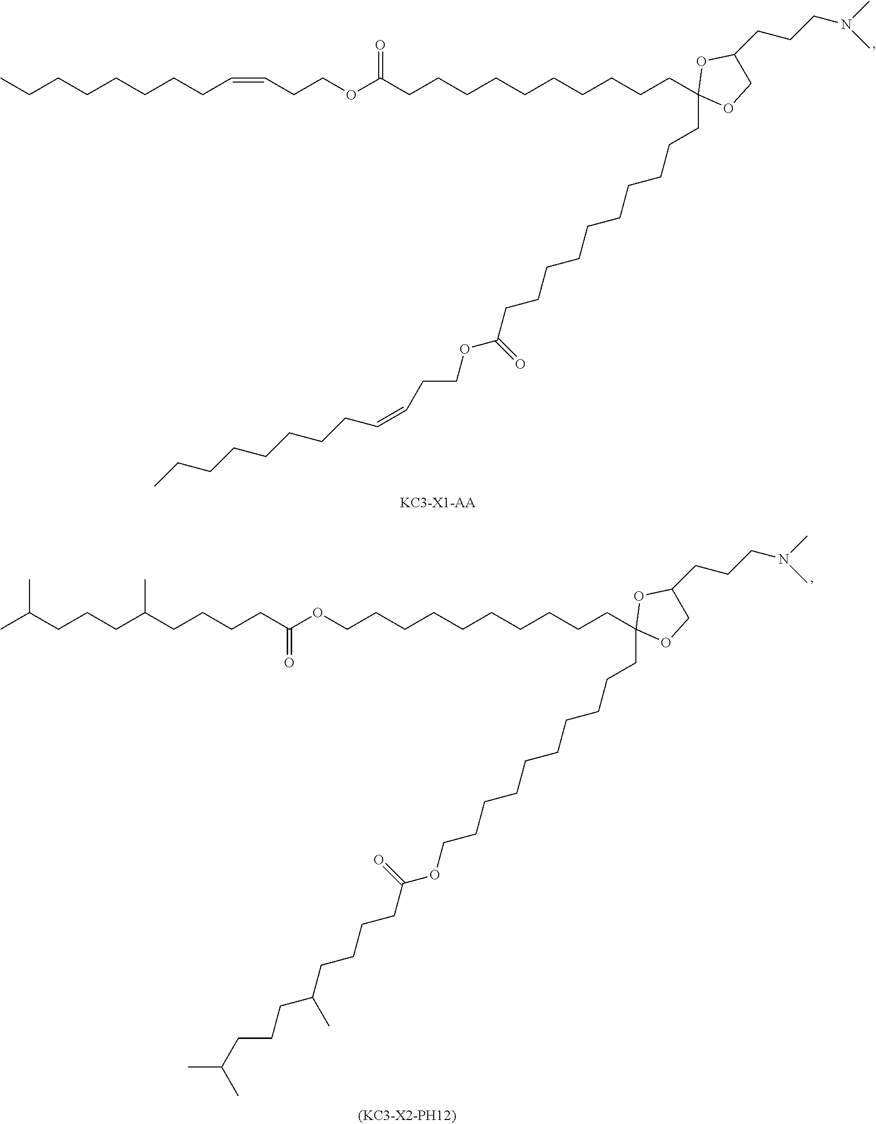

or a salt thereof.

### 2. Ionizable Cationic Lipid Hydrolysis Assay

In some embodiments, ionizable cationic lipids useful for encapsulating mRNA for cellular delivery in a LNP are configured to degrade and clear the body sufficiently rapidly after cellular delivery of the mRNA. In some embodiments, the ionizable cationic lipids can remain stable in vivo long enough to enable efficient cellular uptake and endosomal escape or the encapsulated mRNA, but then break down into non-toxic, excretable metabolites once the encapsulated mRNA has been delivered to a target cell. Ionizable cationic lipids such as SM-102 and ALC-0315 achieve effective delivery but are relatively slow to degrade, contributing to measurable persistence in vivo. An August 2025 study shows the biodistribution patterns of ionizable cationic lipid components of FDA-approved vaccines, including ALC-0315 and SM-102 (Yuxiang R, et al., “Impact of ionizable lipid type on the pharmacokinetics and biodistribution of mRNA-lipid nanoparticles after intravenous and subcutaneous injection,” Journal of Controlled Release, v. 384, 2025, 113945, ISSN 0168-3659). The publication WO2022/115645 (PCT/US2021/060877) discloses other ionizable cationic lipids including KC3-OA.

The term “hydrolysable” (also “hydrolyzable”) refers to compounds, bonds, or functional groups that are able to undergo degradation under intracellular and/or other physiological conditions within the body by means of hydrolysis. A hydrolysable ionizable cationic lipid incorporates bonds (such as ester, carbonate, or amide linkages) that can be cleaved by hydrolysis in aqueous biological environments, often accelerated by enzymes (e.g., esterases). This hydrolytic degradation can result in breakdown into smaller, more biocompatible metabolites, supporting controlled disassembly of the LNP, mRNA release, and improved biodegradability with reduced long-term accumulation. A bioreducible ionizable cationic lipid contains one or more chemical group(s) that are stable under extracellular conditions but are cleaved in the intracellular reductive environment (e.g., high glutathione levels in the cytosol). This reduction-triggered cleavage can lead to lipid degradation or structural rearrangement, promoting endosomal escape and facilitating release of the encapsulated mRNA while also aiding in lipid clearance and reducing toxicity.

One practical way to evaluate and identify ionizable cationic lipids suitable for forming LNP compositions encapsulating mRNA is through a pancreatin assay such as Example 5, which uses a mixture of digestive enzymes to model ester hydrolysis under biologically relevant conditions. Ionizable lipids that are rapidly cleaved in this assay are more likely to undergo efficient enzymatic degradation in vivo, making this a useful screening tool for selecting lipids that maintain delivery performance while clearing from the body more quickly.

Pancreatin contains a mixture of digestive enzymes, including lipases and esterases, that mimic the enzymatic environment encountered by LNPs after administration and cellular uptake. By assessing the susceptibility of candidate ionizable cationic lipids to enzymatic hydrolysis in this assay, it becomes possible to distinguish lipids that undergo controlled degradation from earlier-generation ionizable lipids that were comparatively resistant to hydrolysis. This selective hydrolysability is desirable because it can facilitate LNP disassembly, promote endosomal escape and mRNA release, and enhance biodegradability, thereby reducing lipid accumulation and potential toxicity. Consequently, a pancreatin-based assay serves as a useful screening tool to identify next-generation ionizable cationic lipids with an improved balance of stability during formulation and delivery, and degradability under biologically relevant enzymatic conditions.

Aspects of the disclosure are based in part on the discovery of ionizable lipids containing one or more functionalized aliphatic tails interrupted by biodegradable and/or hydrolysable groups with certain defined ratios of aliphatic chain length on either side of the biodegradable and/or hydrolysable group emerged from systematic structure-activity relationship studies aimed at improving nucleic acid delivery while enhancing biodegradability. Some aspects of the disclosure are based on discovery that positioning an ester within the lipid tail, rather than only at the headgroup-tail junction, allowed control over certain properties of the ionizable lipids. The ionizable lipids with ester-containing, asymmetrically segmented lipid tails are useful in preparing lipid nanoparticle formulations for the delivery of nucleic acids, such as in mRNA vaccine and therapeutic applications.

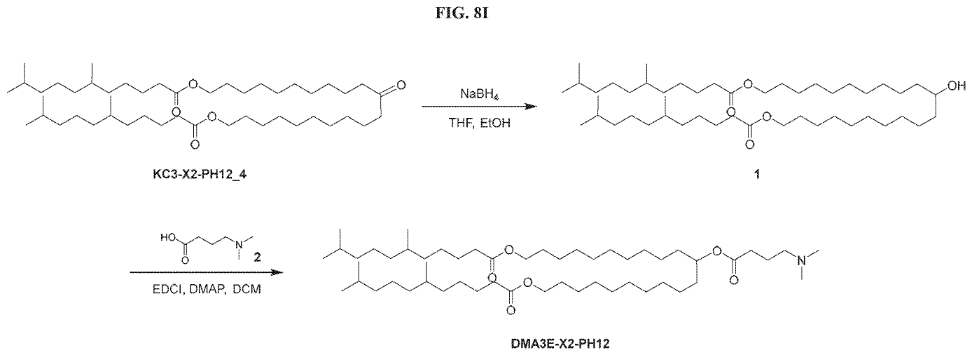

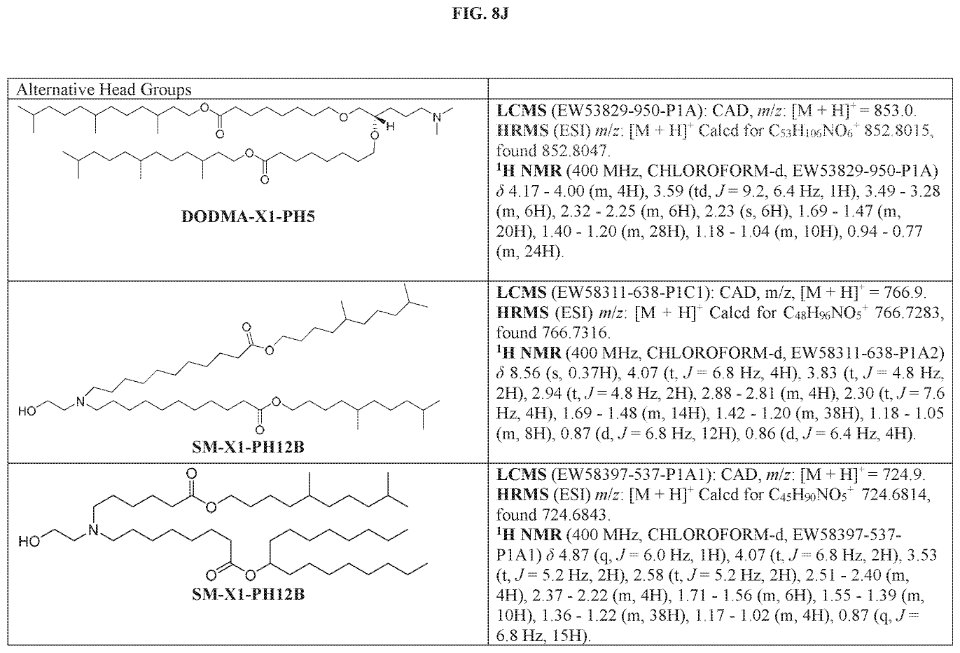

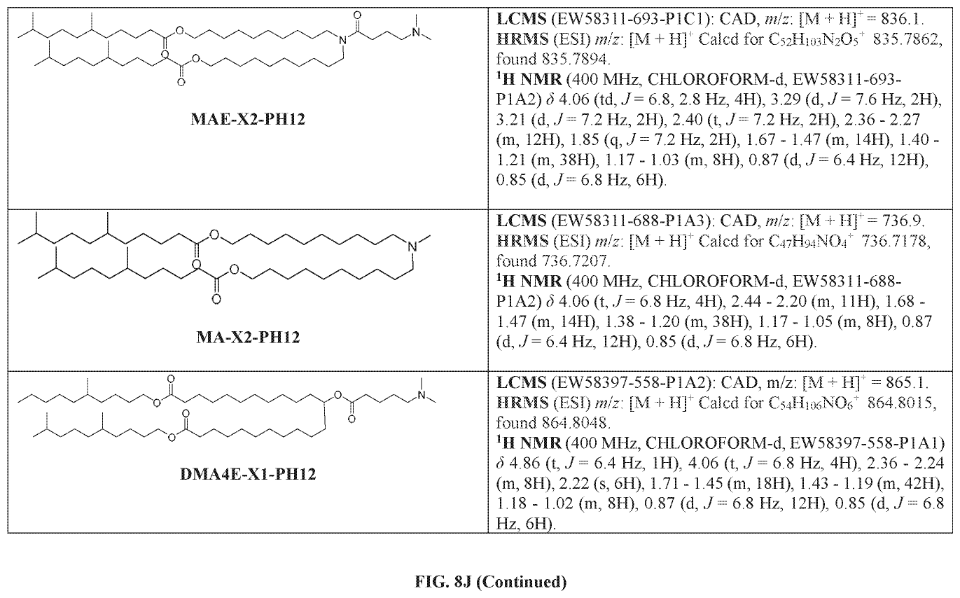

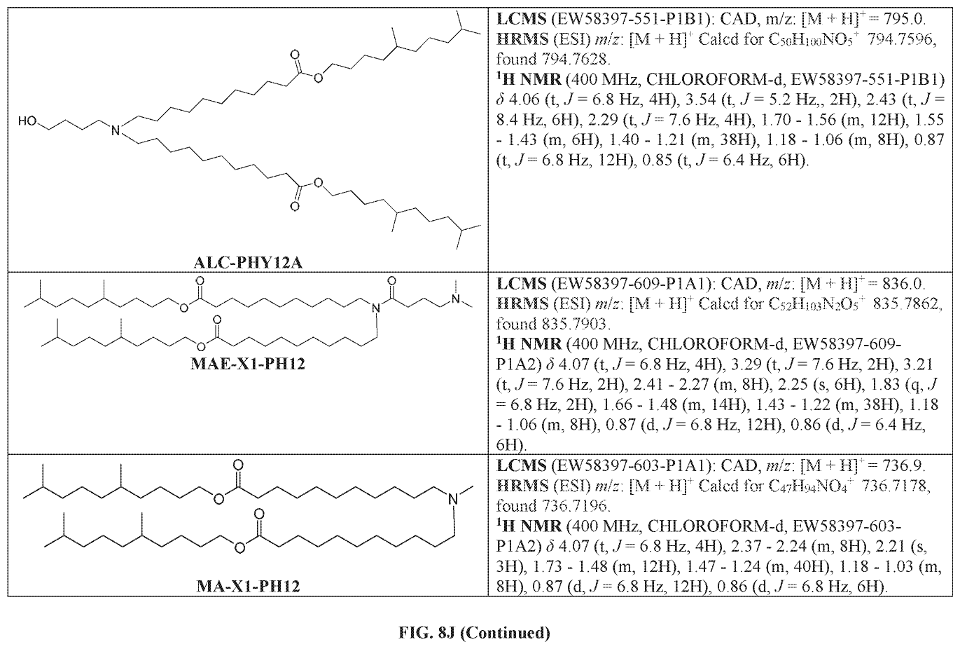

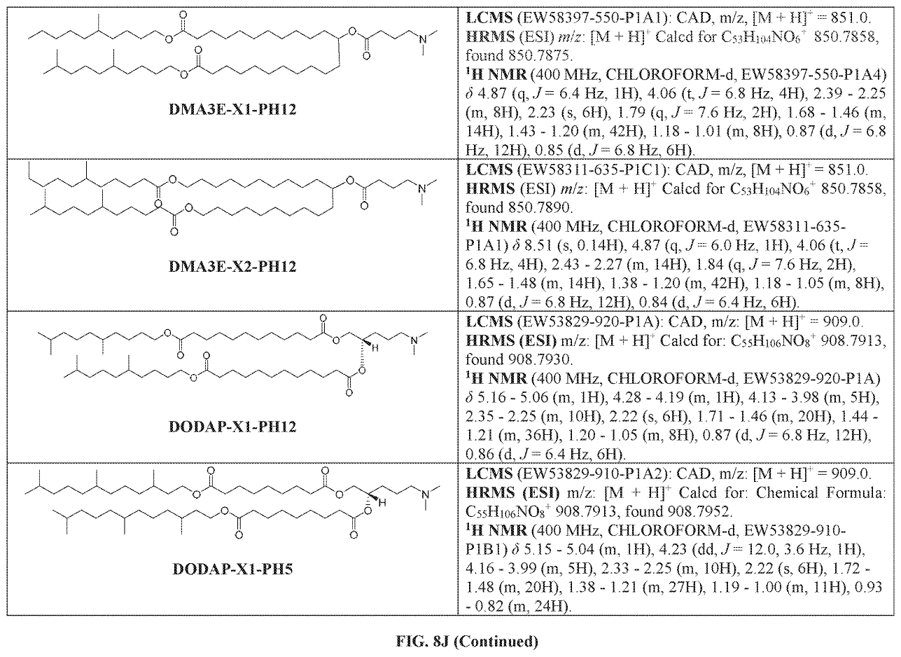

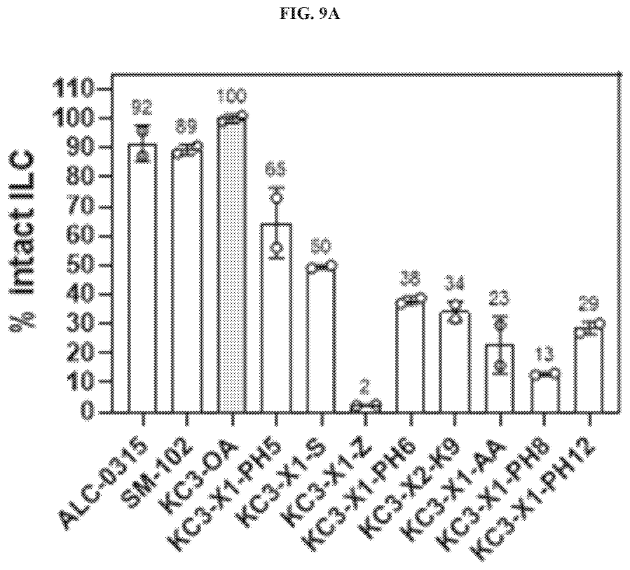

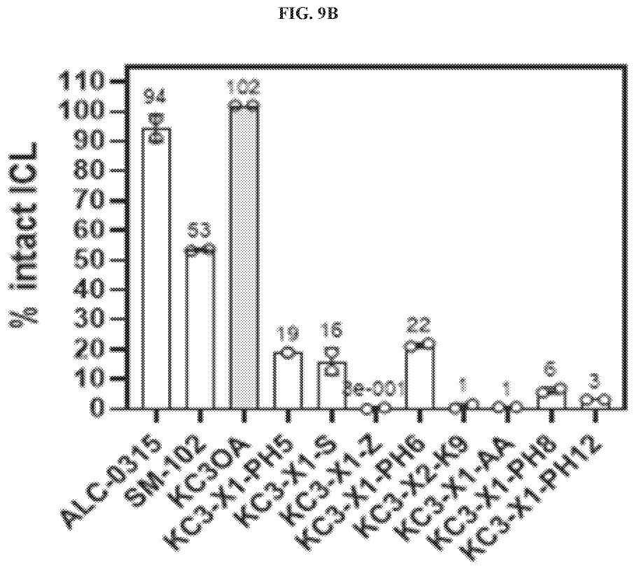

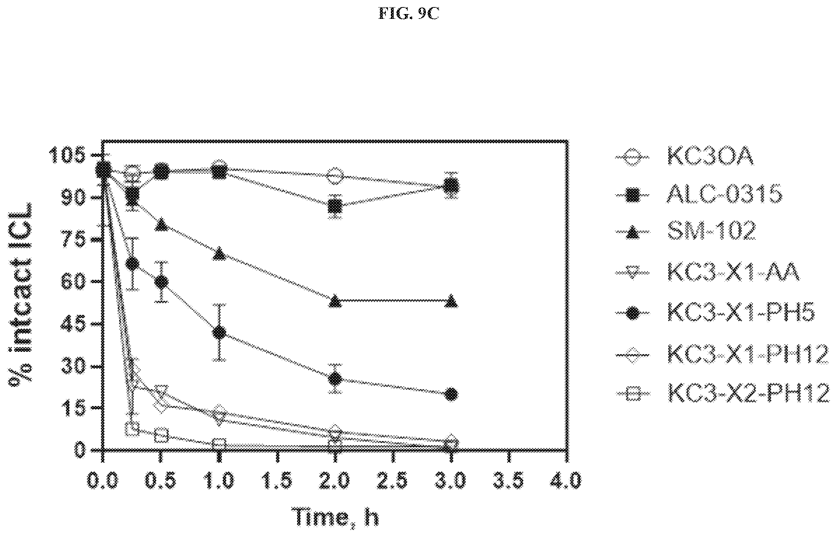

The hydrolysis of various ionizable lipids were evaluated in the pancreatin enzyme assay of Example 5, to obtain the data shown in the graphs of FIGS. 9A, 9B and 9C. FIG. 9C is a graph showing the biodegradation of a variety of ionizable cationic lipids measured every half hour for 3 hours. Pancreatin contains a mixture of digestive enzymes, including lipases and esterases, that mimic the enzymatic environment encountered by LNPs after administration and cellular uptake. FIG. 9A is a graph of the biodegradability of ionizable cationic lipids (ICLs) measured after 15 minutes in the presence of pancreatin enzymes (including trypsin, amylase and lipase) measured according to the assay of Example 5. In FIG. 9A, the compounds of Formula (I) were hydrolyzed about 29%-98% more than ALC-0315, about 27%-98% more than SM-102 and about 35%-98% faster than KC3-OA. FIG. 9B is a graph of the biodegradability of ionizable cationic lipids (ICLs) measured after 3 hours in the presence of pancreatin enzymes (including trypsin, amylase and lipase) measured according to the assay of Example 4. In FIG. 9B, the compounds of Formula (I) were hydrolyzed about 77-99% more than ALC-0315, about 58-99% more than SM-102 and about 81-99% faster than KC3-OA. FIG. 9C is a graph showing the enzymatic degradation of various ionizable cationic lipids. The ionizable lipids of Formula (I) were hydrolyzed significantly faster than ALC-0315 and SM-102 in the assay of Example 5. The hydrolysis of the ionizable lipids of Formula (I) was significantly greater and more rapid than comparator ionizable lipids ALC-0315, SM102 and KC3-OA, when evaluated in an assay incorporating pancreatin enzymes in Example 5.

The chemical structures of the other tested compounds showing greater and more rapid biodegradability than ALC-0315, SM-102 and KC3-OA in the pancreatin assay of Example 5 (designated herein as KC3-X1-PH5, KC3-X1-S, KC3-X1-Z, KC3-X1-PH6, KC3-X2-K9, KC3-X1-AA, KC3-X1-PH8, KC3-X1-PH12, KC3-X2-PH12) all included a pair of functionalized aliphatic tails comprising an ester group positioned between a proximal saturated linear 7-10 carbon alkane chain extending to an alkylamine head group and a distal linear aliphatic chain (either saturated or comprising an olefin) or branched aliphatic chain (comprising one or more 2 MB monomers). In some embodiments, the functionalized aliphatic chains of the ionizable cationic lipid comprise one or more hydrolysable or bioreducible group(s), such as A₁ and A₂ in Formula (I) and Formula (I-A).

In some embodiments, the disclosure provides certain LNP compositions comprising one or more ionizable lipids disclosed herein, such as the ionizable lipids of Formula (I). In some aspects, the LNP compositions comprise a nucleic acid encapsulated in lipid nanoparticles formed from: (a) an ionizable lipid of Formula (I) at a N/P ratio of 3 to 8 relative to the nucleic acid in a total amount of 40-65 mol % of the total lipid content of the LNP composition; (b) a sterol in a total amount of 0.5-50 mol % of the total lipid content of the LNP composition; (c) one or more phospholipids in a total amount of phospholipids of 5-50 mol % of the total lipid content of the LNP composition; and (d) a conjugated lipid in a total amount of 0.5-2.5 mol % of the total lipid content of the LNP composition. In some embodiments, a lipid nanoparticle (LNP) composition comprises a mRNA nucleic acid encapsulated in lipidic nanoparticles formed from the ionizable lipid disclosed herein, at a N/P ratio of 3 to 8 relative to the mRNA nucleic acid in a total amount of 40-65 mol % of the total lipid content of the LNP composition; a sterol in a total amount of 25-45 mol % of the total lipid content of the LNP composition; one or more phospholipids in a total amount of phospholipids of 5-25 mol % of the total lipid content of the LNP composition; and a PEG-containing conjugated lipid in a total amount of 0.5-5.0 mol % of the total lipid content of the LNP composition. In some embodiments, the two or more phospholipids in the LNP composition comprise a total of 5 mol % of a phosphatidyl-L-serine (PS). In some embodiments, the LNP composition comprises the conjugated lipid in a total amount of 0.5-2.5 mol % of the total lipid content of the LNP composition. In some embodiments, the phospholipids consist of: 1,2-distearoyl-sn-glycero-3-phosphocholine (DSPC) in a total amount of 5 mol % of the total lipid content of the LNP composition, and a phosphatidyl-L-serine (DPPS), such as dipalmitoylphosphatidyl-L-serine (DPPS) or distearoylphosphatidyl-L-serine (DSPS), in a total amount of 5 mol % of the total lipid content of the LNP composition. In some embodiments, the PEG-containing conjugated lipid in the LNP is PEG(2000)-dimyristoylglycerol (PEG-DMG) or PEG(2000)-dilauroylglycerol (PEG-DLG).

### 3. Lipid Nanoparticle Compositions

In some embodiments, lipid nanoparticle (LNP) compositions comprising the ionizable lipids disclosed herein. In some embodiments, a lipid nanoparticle (LNP) composition comprises a nucleic acid such as mRNA encapsulated in lipid nanoparticles formed from: an ionizable cationic lipid disclosed herein, one or more phospholipids, a sterol and a PEG conjugated lipid. In some embodiments, the ionizable cationic lipid can become positively charged in acidic conditions within a cell to enable efficient complexation and endosomal escape of the encapsulated nucleic acid, but remains neutral at physiological pH to improve tolerability. The phospholipids provide structural support and help define the lipid nanoparticles. The sterol, commonly cholesterol, stabilizes the lipid nanoparticles by modulating membrane fluidity and packing. Finally, the PEG-lipid is included to control particle size, reduce aggregation, and extend circulation time by providing a hydrophilic barrier. Together, these components can self-assemble under appropriate conditions into a stable lipid nanoparticle capable of encapsulating the nucleic acid, navigating biological environments, and delivering the encapsulated nucleic acid payload into target cells. The lipid nanoparticles can encapsulate a therapeutic agent such as a nucleic acid (e.g., mRNA) encoding a protein, a small molecule therapeutic or one or more other therapeutic agent(s). Release of nucleic acids from LNP formulations, among other characteristics such as liposomal clearance and circulation half-life, can be modified by the presence of polyethylene glycol and/or sterols (e.g. cholesterol) or other potential additives in the LNP, as well as the overall chemical structure, including pKa of any ionizable cationic lipid included as part of the formulation.

3a. Ionizable Cationic Lipid

Suitable ionizable cationic lipids (ICLs) for mRNA encapsulation are typically amphiphilic molecules engineered with a protonatable amine headgroup, biodegradable linker (often ester-based), and hydrophobic alkyl tails that promote nanoparticle self-assembly and membrane interaction. Clinically validated examples include ALC-0315 and SM-102, two ionizable lipids used in currently FDA-approved mRNA lipid nanoparticle formulations, along with related research-stage analogs such as DM3-type structures. These lipids are designed to be near-neutral at physiological pH but become positively charged in acidic environments, enabling efficient electrostatic complexation with negatively charged mRNA during formulation while minimizing systemic toxicity in circulation. Their branched or cone-shaped hydrophobic tails facilitate formation of nonlamellar phases that enhance endosomal escape and cytosolic release of mRNA after cellular uptake. In lipid nanoparticles, these ICLs form the functional core, protecting mRNA from degradation, promoting cellular uptake, and enabling intracellular delivery; differences in structure, pKa, and biodegradability influence pharmacokinetics, biodistribution, and expression efficiency. Overall, effective ICLs balance ionization behavior, structural dynamics, and metabolic clearance to optimize mRNA loading, stability, and delivery performance.

In some embodiments, the ionizable cationic lipid is KC2 ionizable lipid. KC2 is an ionizable cationic lipid in the DLin-KC3 family that incorporates dilinoleyl hydrophobic tails, a tertiary amine headgroup, and a characteristic dioxolane (ketal/acetal) linker substructure that contributes to its pH-responsive behavior-remaining near neutral at physiological pH but becoming protonated in acidic environments to enable efficient RNA complexation and endosomal escape. The KC3 framework was first described by Scott C. Semple Semple, S. C., Akinc, A., Chen, J., Sandhu, A. P., Mui, B. L., Cho, C. K., Sah, D. W. Y., Stebbing, D., Crosley, E. J., Yaworski, E., Hafez, I. M., Dorkin, J. R., Qin, J., Lam, K., Rajeev, K. G., Wong, K. F., Jeffs, L. B., Nechev, L., Eisenhardt, M. L., . . . Hope, M. J. Rational design of cationic lipids for siRNA delivery. Nature Biotechnology 28(2), 172-176 (2010). https://doi.org/10.1038/nbt.1602.

In some embodiments, the ionizable cationic lipid is KC3ionizable lipid. KC3-OA, as described in PCT publication WO 2023/230587, is an ionizable cationic lipid defined structurally as a dioxolane-containing amine with two unsaturated C18 (oleyl) hydrophobic chains-formally, a 2,2-di((Z)-octadec-9-en-1-yl)-substituted 1,3-dioxolane linked to a dimethylaminopropyl headgroup. It is disclosed as either a racemic mixture or as purified (R)/(S) enantiomers and is used as the primary ionizable lipid component in lipid nanoparticles, typically comprising ~40-65 mol % of the formulation alongside cholesterol, phospholipids, and PEG-lipids. The patent emphasizes its role in enabling efficient nucleic acid (including mRNA) delivery, with the dioxolane core and tertiary amine supporting pH-dependent ionization and formulation into LNP systems with defined N/P ratios and immunologically active payloads.

3b. Sterol

In some embodiments, the LNP comprises a sterol or ionizable sterol molecule. A sterol is a lipid that comprises a polycyclic structure and an optionally a hydroxyl or ether substituent, and may be naturally occurring or non-naturally occurring (e.g., a synthetic sterol). Sterols may comprise no double bonds, a single double bond, or multiple double bonds. Sterols may further comprise an alkyl, alkenyl, halo, ester, ketone, hydroxyl, amine, polyether, carbohydrate, or cyclic moiety. An exemplary listing of sterols includes cholesterol, dehydroergosterol, ergosterol, campesterol, β-sitosterol, stigmasterol, lanosterol, dihydrolanosterol, desmosterol, brassicasterol, lathosterol, zymosterol, 7-dehydrodesmosterol, avenasterol, campestanol, lupeol, and cycloartenol. In some embodiments, the sterol comprises cholesterol, dehydroergosterol, ergosterol, campesterol, β-sitosterol, or stigmasterol. Additional sterols that may be included in an LNP described herein are disclosed in Fahy, E. et al. (J. Lipid. Res. 46:839-862 (2005). In some embodiments, the sterol is cholesterol. In some embodiments, the sterol is dehydroergosterol. In some embodiments, the sterol is ergosterol. In some embodiments, the sterol is campesterol. In some embodiments, the sterol is β-sitosterol. In some embodiments, the sterol is stigmasterol. In some embodiments, the sterol is a corticosteroid. (e.g., corticosterone, hydrocortisone, cortisone, or aldosterone).

In lipid nanoparticles (LNPs), the sterol (most commonly cholesterol) is included for membrane stability, but its amount must be balanced so it does not interfere with nanoparticle formation or function. The sterol concentration in the lipid nanoparticle can be sufficient to stabilize the particle structure by promoting proper lipid packing, preventing payload leakage, and maintaining integrity during storage and in biological fluids. An adequate amount of sterol also supports effective endosomal escape, since certain sterol levels enhance membrane fusion. However, the sterol level should not be so high that it reduces membrane fluidity to the point that the ionizable lipid cannot reorganize during endosomal escape. Excess sterol can also decrease encapsulation efficiency by disrupting the formation of the internal structures required for loading nucleic acids, and it may interfere with proper self-assembly, leading to malformed particles or precipitation. Furthermore, too much sterol can displace helper phospholipids that are needed to achieve appropriate curvature and fusogenic activity. Thus, the sterol level must be balanced—high enough to stabilize the nanoparticle, but not so high that it rigidifies the membrane or hinders assembly or functional performance. In some embodiments, the LNP composition comprises a total of 25-40 mol % cholesterol.

3c. Phospholipid(s)

In some embodiments, the LNP comprises a phospholipid. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol.

In some embodiments, the lipid nanoparticles comprise one or more phospholipids, optionally including a neutral phospholipid and an anionic phospholipid. A phospholipid is a lipid that comprises a phosphate group and at least one alkyl, alkenyl, or heteroalkyl chain. A phospholipid may be naturally occurring or non-naturally occurring (e.g., a synthetic phospholipid). A phospholipid may comprise an amine, amide, ester, carboxyl, choline, hydroxyl, acetal, ether, carbohydrate, sterol, or a glycerol. In some embodiments, a phospholipid may comprise a phosphocholine, phosphosphingolipid, or a plasmalogen. Exemplary phospholipids include 1,2-dioleoyl-sn-glycero-3-phosphocholine (DOPC), 1,2-dipalmitoyl-sn-glycero-3-phosphocholine (DPPC), 1,2-dioleoyl-sn-glycero-3-phosphoethanolamine (DOPE), 1,2-distearoyl-sn-glycero-3-phosphocholine (DSPC), hydrogenated soy phosphatidylcholine (HSPC), 1,2-dilauroyl-sn-glycero-3-phosphocholine (DLPC), 1,2-dimyristoyl-sn-glycero-3-phosphocholine (DMPC), 1,2-distearoyl-sn-glycero-3-phosphoethanolamine (DSPE), 1-myristoyl-2-oleoyl-sn-glycero-3-phosphocholine (MOPC), 1,2-diarachidonoyl-sn-glycero-3-phosphocholine (DAPC), 1-palmitoyl-2-linoleoyl-sn-glycero-3-phosphatidylcholine (PLPC), 1-palmitoyl-2-oleoyl-glycero-3-phosphocholine (POPC), 1-stearoyl-2-myristoyl-sn-glycero-3-phosphocholine (SMPC), 1-palmitoyl-2-myristoyl-sn-glycero-3-phosphocholine (PMPC), bis(monoacylglycerol)phosphate (BMP), L-α-phosphatidylcholine, 1,2-Diheptadecanoyl-sn-glycero-3-phosphorylcholine (DHDPC), and 1-stearoyl-2-arachidonoyl-sn-glycero-3-phosphocholine (SAPC). Additional phospholipids that may be included in an LNP described herein are disclosed in Li, J. et al. (Asian J. Pharm. Sci. 10:81-98 (2015)), which is incorporated herein by reference in its entirety. In some embodiments, the neutral phospholipid is 1,2-distearoyl-sn-glycero-3-phosphocholine (DSPC).

In some embodiments, the one or more phospholipids include an anionic phospholipid, optionally selected to target dendritic cells. Anionic phospholipids useful as targeting lipids for LNPs include phosphatidylserine (PS), phosphatidylglycerol (PG), phosphatidic acid (PA), N-glutaryl-phosphatidylethanolamine (N-Glu-PE), N-succinyl-phosphatidylethanolamine (N-Suc-PE), and cardiolipin. Examples include DSPG, DPPG, N-Suc-DSPE, N-Glu-DSPE, DSPA, and cardiolipin. Additional anionic lipids include DMPS, DPPS, DOPS, and DSPS (L- or D-isomers). In some embodiments, the anionic targeting ligand is PS, PG, N-Glu-PE, or N-Suc-PE. The anionic phospholipid may be phosphatidylserine, including the L-isomer, with fully saturated acyl chains such as DMPS, DPPS, or DSPS.

In some embodiments, a phospholipid may comprise a phosphocholine, phosphosphingolipid, or a plasmalogen. Exemplary phospholipids include 1,2-distearoyl-sn-glycero-3-phosphocholine (DSPC), 1,2-dipalmitoyl-sn-glycero-3-phosphocholine (DPPC), dipalmitoylphosphatidyl-L-serine (DPPS), distearoylphosphatidyl-L-serine (DSPS), hydrogenated soy phosphatidylcholine (HSPC), and/or sphingomyelin (SM). Additional phospholipids that may be included in an LNP described herein are disclosed in Li, J. et al. (Asian J. Pharm. Sci. 10:81-98 (2015)), which is incorporated herein by reference in its entirety.

The concentration of one or more phospholipids in a lipid nanoparticle can be selected to form and maintain the structural framework of the lipidic particle. At an effective level, phospholipids contribute to membrane integrity, support proper curvature during self-assembly, and facilitate stable encapsulation of the therapeutic payload. Phospholipids also modulate membrane fluidity and fusogenic behavior, enabling efficient interaction with cellular membranes and promoting endosomal escape when required. However, phospholipids should not be present at such high levels that they dominate the formulation and reduce the contribution of the ionizable lipid or sterol, which can impair nucleic-acid loading, alter particle morphology, or diminish delivery efficiency. Excessive phospholipid content can also produce overly rigid or overly lamellar structures that are not optimal for nanoparticle uptake or intracellular trafficking. Accordingly, the phospholipid concentration must be balanced-high enough to support particle formation, stability, and functional membrane interactions, but not so high that it interferes with encapsulation, self-assembly, or delivery performance.

In some embodiments, the LNP comprises one or more phospholipids in a total amount of phospholipids of 5-20 mol % of the total lipid content of the LNP composition, optionally including 5-10 mol % of an anionic lipid. In some embodiments, the LNP comprises a neutral phospholipid and an anionic lipid in equal amounts. For example, in some embodiments, the LNP comprises a neutral phospholipid in a total amount of 5-10 mol % of the total lipid content of the LNP composition, and an anionic lipid in a total amount of 5-10 mol % of the total lipid content of the LNP composition. In some aspects, the one or more phospholipids in the LNP comprise a phosphatidylserine (PS) lipid in a total amount of 2.5-10 mol % of the total lipid content of the LNP composition. In some aspects, the phosphatidylserine (PS) lipid in the LNP consists of, consists essentially of or comprises dipalmitoylphosphatidyl-L-serine ((L-serine)DPPS). In some aspects, the one or more phospholipids in the LNP comprise a phospholipid selected from the group consisting of: distearoylphosphatidylcholine (DSPC) and hydrogenated soy phosphatidylcholine (HSPC). In some aspects, the one or more phospholipids in the LNP consist of distearoylphosphatidylcholine (DSPC) and dipalmitoylphosphatidyl-L-serine ((L-serine)DPPS).

In some embodiments, the ionizable cationic lipid to cholesterol molar ratios is from about 65:35 to 40:60. In some embodiments, the ICL to cholesterol molar ratio is from about 60:40 to about 45:55. In some embodiments, the phosphatidylcholine to cholesterol molar ratio is from about 1:5 to about 1:2. In some embodiments, the lipidic nanoparticle comprises ICL, DSPC, cholesterol and polymer-conjugated lipid in a about 49.5:10.3:39.6:2.5 molar ratio.

3d. Conjugated Lipid

In some embodiments, the LNP comprises an alkylene glycol-containing lipid. An alkylene glycol-containing lipid is a lipid that comprises at least one alkylene glycol moiety, for example, a methylene glycol or an ethylene glycol moiety. In some embodiments, the alkylene glycol-containing lipid comprises a polyethylene glycol (PEG). In some embodiments, the LNP comprises a conjugate lipid that is a PEG-containing lipid comprising an amine, amide, ester, carboxyl, phosphate, choline, hydroxyl, acetal, ether, heterocycle, or carbohydrate. An alkylene glycol-containing lipid may be a PEG-containing lipid. Polymer-conjugated lipids may include poly(ethylene glycol)-conjugated (pegylated)phospholipids (PEG-lipids). PEG-containing lipids may comprise at least one alkyl or alkenyl group, e.g., greater than six carbon atoms in length (e.g., greater than about 8 carbons, 10 carbons, 12 carbons, 14 carbons, 16 carbons, 18 carbons, 20 carbons or more in length), e.g., in addition to a PEG moiety. In an embodiment, a PEG-containing lipid comprises a PEG moiety comprising at least 20 PEG monomers, e.g., at least 30 PEG monomers, 40 PEG monomers, 45 PEG monomers, 50 PEG monomers, 100 PEG monomers, 200 PEG monomers, 300 PEG monomers, 500 PEG monomers, 1000 PEG monomers, or 2000 PEG monomers. Exemplary PEG-containing lipids include PEG-DMG (e.g., DMG-PEG2k), PEG-c-DMG, PEG-DSG, PEG-DPG, PEG-DSPE, PEG-DMPE, PEG-DPPE, PEG-DOPE, and PEG-DLPE. In some embodiments, the PEG-lipids include PEG-DMG (e.g., DMG-PEG2k), PEG-c-DMG, PEG-DSG, and PEG-DPG. The molecular weight of the PEG portion in the PEG-lipid component can also vary from 500-10,000 g/mol, from 1,500-6000 g/mol, but is preferably about 2,000 MW. Additional PEG-lipids that may be included in an LNP described herein are disclosed in Fahy, E. et al. (J. Lipid. Res. 46:839-862 (2005) which is incorporated herein by reference in its entirety. In some embodiments, the PEG-lipid is PEG-DMG (e.g., DMG-PEG2k). In some embodiments, the PEG-lipid is α-(3′-{[1,2-di(myristyloxy)propanoxy]carbonylamino}propyl)-ω-methoxy, polyoxyethylene (PEG-c-DMG). In some embodiments, the LNP composition comprises 1.5-4.0 mol % PEG-containing conjugated lipid in the LNP composition is PEG-DMG. In some aspects, the PEG-containing conjugated lipid in the LNP is PEG(2000)-dimyristoylglycerol (PEG-DMG). In some aspects, the PEG-containing conjugated lipid in the LNP is 1,2-dilauroyl-rac-glycero-3-methoxypolyethylene glycol-2000 (PEG-DLG).

3e. Encapsulated Nucleic Acid

In some embodiments, LNP compositions comprising lipid nanoparticles comprising the ionizable lipid(s) disclosed herein and encapsulating mRNA. Typically, an mRNA comprises a 5′-UTR, a protein coding region, and a 3′-UTR. According to some embodiments, the RNA does not have uncapped 5′-triphosphates. Removal of such uncapped 5′-triphosphates can be achieved by treating RNA with a phosphatase. In some embodiments, the RNA can be provided with a 5-cap or 5′-cap analog. The term “5-cap” refers to a cap structure found on the 5′-end of an mRNA molecule and generally consists of a guanosine nucleotide connected to the mRNA via an unusual 5′ to 5 triphosphate linkage. In some embodiments, this guanosine is methylated at the 7-position. The RNA according to aspects of the disclosure may have modified ribonucleotides in order to increase its stability and/or decrease cytotoxicity. For example, in some embodiments, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine. In other embodiments, the mRNA does not include N1-methyl-pseudouridine.

The RNA according to aspects of the disclosure may have modified ribonucleotides in order to increase its stability and/or decrease cytotoxicity. For example, in some embodiments, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine. In other embodiments, the mRNA does not include N1-methyl-pseudouridine.

Exemplary LNP formulations can deliver mRNA encoding any protein antigen derived from an infectious pathogen or disease that can be targeted with the immune system. Listed in Tables D and E is a non-exhaustive list of vaccine antigens that could be encoded by mRNA and encapsulated in LNPs to comprise a vaccine using the lipids, methods, and formulations described in the Examples.

TABLE D
Examples of antigens that could be encoded by mRNA and
encapsulated in LNPs to comprise a novel vaccine.

| Infectious | vaccine antigen | Reference |
|---|---|---|
| pathogen/disease | description | (PMID) |
| respiratory | Prefusion stabilized F (DS-Cav1) and | 24179220, 27478931, |
| syncytial | subsequent iterative structural | 30849373, 37689061 |
| virus (RSV) | improvements. E.g. sc9-10 DS-Cav1 | |
| | A149C Y458C, DS-Cav1-153-50A | |
| | SEQ ID NO: 9 | |
| | RSV-G antigen | |
| | SEQ ID NO: 509 | |
| | SEQ ID NO: 510 | |
| | SEQ ID NO: 511 | |
| | SEQ ID NO: 512 | |
| | SEQ ID NO: 513 | |
| | SEQ ID NO: 515 | |
| Influenza | HA (e.g., SEQ ID NO: 5, SEQ ID NO: 12, | 26301691, 26303961, |
| virus | SEQ ID NO: 14, SEQ ID NO: 915, SEQ ID | 30808695, 36356572, |
| (IAV, IBV) | NO: 905-907, and/or SEQ ID NO: 18); | 35869085, 35383176, A |
| | HA stabilized stem (Group 1 and 2), | Dosey et al. bioRxiv |
| | neuraminidase (NA) | 2025.07.30.667762 |
| | (e.g. SEQ ID NO: 37, SEQ ID NO: 38, | |
| | SEQ ID NO: 39); M2; stabilized H5 (e.g., | |
| | SEQ ID NO: 15), | |
| Lassa | stabilized LASV GPC; nucleoprotein; | 38177144, 32269122, |
| virus | NP-GPC chimeric fusion protein (e.g., | 32142546 |
| (LASV) | SEQ ID NO: 801 or SEQ ID NO: 803) | |
| human | gp 120 eOD-GT-based immunogens (e.g. | 23539181, 31515132, |
| immunodeficiency | eOD-GT8), stabilized Env trimers (e.g. | 27174988, 37098956, |
| virus (HIV) | BG505 DS-SOSPI), conserved protease | 38997302, 33766848, |
| | cleavage sites, gp41 fusion peptide, HLA- | 26687358 |
| | E-restricted epitopes | |
| Mycobacterium | individual Mtb proteins (e.g. ESAT-6, | 31114037, 36604540, |
| tuberculosis | CFP10, Ag85B, Ag85A Esx family | 15187142, 24349004, |
| (tuberculosis) | proteins), | 22133873, 34795205, |
| | SEQ ID NO: 444 | 29263854, 29996082; N |
| | SEQ ID NO: 445 | Agrawal et al. 2025, |
| | SEQ ID NO: 447 | bioRxiv |
| | SEQ ID NO: 449 | 2025.09.30.679428 |
| | SEQ ID NO: 450 | |
| | synthetic fusion proteins (e.g. M72, H4, | |
| | H56, H65, H107, CysVac2, BNT164B1/2) | |
| | SEQ ID NO: 401 | |
| | SEQ ID NO: 403 | |
| | SEQ ID NO: 405 | |
| | SEQ ID NO: 407 | |
| | SEQ ID NO: 409 | |
| | SEQ ID NO: 411 | |
| | SEQ ID NO: 413 | |
| | SEQ ID NO: 415 | |
| | SEQ ID NO: 417 | |
| | SEQ ID NO: 419 | |
| | SEQ ID NO: 421 | |
| | SEQ ID NO: 423 | |
| | SEQ ID NO: 425 | |
| | SEQ ID NO: 427 | |
| | SEQ ID NO: 429 | |
| | SEQ ID NO: 431 | |
| | SEQ ID NO: 437 | |
| | SEQ ID NO: 438 | |
| | SEQ ID NO: 440 | |
| | SEQ ID NO: 442 | |
| | SEQ ID NO: 451 | |
| Plasmodium | circumsporozoite protein (CPS), RTS, S, | 27355532, 33964223, |
| falciparim | R21, CSP-HBsAg fusion (hepatitis B | 37168860, 35822551, |
| (malaria) | surface Ag); Pfs25; PfGARP; CelTOS, | 27457156, 39019011, |
| | RH5.1, RH5.2 | 39025972, |

TABLE E
Examples of antigens that could be encoded by mRNA and
encapsulated in LNPs to comprise a novel vaccine.

| Infectious pathogen/disease | vaccine antigen description | Reference (PMID) |
|---|---|---|
| respiratory syncytial | RSVpreF (DS-Cav1) and subsequent | 24179220, 27478931, |
| virus (RSV) | iterative structural improvements. | 30849373, 37689061 |
| | E.g. sc9-10 DS-Cav1 A149C | |
| | Y458C, DS-Cav1-153-50A | |
| Influenza virus | HA, HA stabilized stem, NA, M2 | 26301691, 26303961, |
| (IAV, IBV) | | 30808695, 36356572, |
| | | 35869085, 35383176, |
| | | 3480945 |
| Lassa virus | stabilized LASV GPC, | 38177144, 32269122, |
| (LASV) | nucleoprotein | 32142546 |
| SARS-CoV-2, | spike (S) protein, nucleocapsid, | 33128877 |
| betacoronaviruses | membrane, ORF3a, ORF1ab | |
| Middle East respiratory | spike (S) glycoprotein | 28807998 |
| syndrome coronavirus | | |
| (MERS-CoV) | | |
| Ebola virus (EBOV) | glycoprotein (GP), | 32418798, 31589088, |
| | nucleoprotein (NP) | 32234486 |
| Marburg virus (MARV) | glycoprotein (GP), | 31589088, 32234486 |
| | nucleoprotein (NP) | |
| human metapneumovirus | F glycoprotein | 34551978 |
| (HMPV) | | |
| parainfluenza virus | Type 1 and type 4. Prefusion | 30420505 |
| | stabilized fusion proteins (e.g. | |
| | prefusion PIV3 F) | |
| human immunodeficiency | gp120 eOD-GT-based immunogens | 23539181, 31515132, |
| virus (HIV) | (e.g. eOD-GT8), stabilized Env | 27174988 |
| | trimers (e.g. BG505 DS-SOSPI), | |
| | conserved protease cleavage sites, | |
| | gp41 fusion peptide | |
| human papilloma | L1 capsid protein, E7, glycoprotein | 38462683, 36867683 |
| viruses (HPV) | D, gDE7 fusion | |
| Zika virus (ZIKV) | prM-E | 28222903 |
| Powassan virus (POWV) | prM, prE | 30566864 |
| MPox virus | A35, B6, M1, H3 | 38366591 |
| norovirus | capsid protein VP1 | 38811605 |
| human cytomegalovirus | glycoprotein B (gB), gH, gL, | 38324766, 29456015 |
| (HCMV) | UL128, UL130, UL131 | |
| Ebstein Barr | glycoprotein gp350, gp42, gH/gL | 38479361, 38926438 |
| virus (EBV) | (gp42/gp350) | |
| human varicella-zoster | envelope glycoprotein E (gE) | 25916341 |
| virus (VZV) | | |
| Rabies virus (RABV) | glycoprotein (G) | 37349310 |
| West Nile virus (WNV) | premembrane (prM), envelope (E) | 37125778 |
| Dengue virus (DENV) | envelope protein (E), premembrane | 35608749 |
| | (prM), NS1, NS3, NS5, NS4B | |
| Chikungunya virus | E1, E2, E3 glycoproteins | 23670192, 38005854, |
| (CHIKV). other | | 36474012 |
| Alphaviruses of | | |
| the Togaviridae family | | |
| (RRV, ONNV, BFV, | | |
| MAYV, SINV, EEEV, | | |
| WEEV, VEEV) | | |
| Nipah virus (NiV) | glycoprotein (G) | 38726013 |
| Mycobacterium | individual Mtb proteins (e.g. ESAT- | 31114037, 36604540 |
| tuberculosis (tuberculosis) | 6, CFP10, Ag85B, Esx family | |
| | proteins), synthetic fusion proteins | |
| | (e.g. M72) | |
| Plasmodium | circumsporozoite protein (CPS); | 27355532, 33964223, |
| falciparim(malaria) | RTS,S; CSP-HBsAg fusion | 37168860 |
| | (hepatitus B surface Ag); Pfs25; | |
| | PfGARP; CelTOS | |
| Borrelia | outer surface protein A (OspA) | 37533256 |
| burgdorferi(Lyme | | |
| disease) | | |
| cancer | cancer neoantigens | 37165196, 28678784, |
| | | 35970920, 38538867 |
| A. americanum (alpha-gal | Salivary proteins (e.g. Salp- | 34788080, 37407406 |
| hypersensitivity), I. | family proteins, TSLPI) | |
| scapularis | | |
| Mumps (MuV) | Fusion glycoprotein F, prefusion F | 39527740, |
| | variants, HN | PCT/US2020/064619, |
| | | WO2021119497A1 |
| Measles (MeV) | Fusion glycoprotein F, prefusion F | PCT/US2020/064619, |
| | | WO2021119497A1 |

In some embodiments, the mRNA is a nucleic acid comprising or consisting of SEQ ID NOS: 3, 4, 6, 8, 10, 13, 16, 17, 402, 404, 406, 408, 410, 412, 414, 416, 418, 420, 422, 424, 426, 428, 430, 432, 439, 441, 443, 446, 448, 452, 501, 502, 505, 506, 507, 508, 514, 516, 802, 804, 901, 902, 903, 904, 909, or 914.

In some embodiments, the mRNA encodes a peptide or a protein comprising or consisting of SEQ ID NOS: 2, 5, 7, 9, 12, 14, 15, 18, 36, 37, 38, 39, 401, 403, 405, 407, 409, 411, 413, 415, 417, 419, 421, 423, 425, 427, 429, 431, 433, 434, 437, 438, 440, 442, 444, 445, 447, 449, 450, 451, 509, 510, 511, 512, 513, 515, 801, 803,905, 906, 907, 908, 910, 911, 912 or 915.

In some embodiments, the present disclosure provides certain compounds useful as ionizable lipids in the preparation of lipid nanoparticles encapsulating a nucleic acid such as mRNA. Aspects of the disclosure are based in part on the discovery of ionizable lipids containing one or more functionalized aliphatic tails interrupted by ester linkages with defined ratios of aliphatic chain length on either side of the ester emerged from systematic structure-activity relationship studies aimed at improving nucleic acid delivery while enhancing biodegradability. Applicants discovered that positioning an ester within the lipid tail, rather than only at the headgroup-tail junction, allowed control over certain properties of the ionizable lipids. The ionizable lipids with ester-containing, asymmetrically segmented lipid tails are useful in preparing lipid nanoparticle formulations for the delivery of nucleic acids, such as in mRNA vaccine and therapeutic applications.

In some embodiments, LNP compositions comprising lipid nanoparticles comprising the ionizable lipid(s) disclosed herein and encapsulating mRNA. FIG. 23A is a schematic of a mRNA construct comprising a 5′-cap, a 5′-UTR, a coding sequence (CDS) that encodes a polypeptide antigen, a 3′-UTR and a poly-A region. The CDS can encode a natural or synthetic polypeptide antigen. In some embodiments, the mRNA (100) is a single mRNA comprising a 5′ region (110) and a 3′ region (190) flanking a CDS (150). The 5′ region of the mRNA (110) can comprise a 5′ cap and a 5′ UTR region. The 3′ region of the mRNA (190) can comprise a 3′UTR (182) and a polyA sequence (184). In one embodiment, the mRNA CDS (150) comprises (from 5′ to 3′) a secreted protein consisting of a signal peptide (SP), and one or more unique polypeptides optionally connected by a spacer. In another embodiment, the mRNA CDS (150) comprises (from 5′ to 3′) a type I single-pass membrane protein consisting of a signal peptide (SP), one or more unique polypeptide optionally connected by a spacer, and a terminal domain comprising a transmembrane and optionally a cytoplasmic domain. In another embodiment, the mRNA CDS (150) comprises (from 5′ to 3′) a type II single-pass membrane protein consisting of a transmembrane domain and one or more unique polypeptides optionally connected by a spacer. In another embodiment, the mRNA CDS (150) comprises (from 5′ to 3′) a type III single-pass membrane protein consisting of an extracellular domain, one or more unique polypeptides optionally connected by a spacer, a transmembrane domain and a intracellular domain comprised of one or more unique polypeptides optionally connected by a spacer. In another embodiment, the mRNA CDS (150) comprises (from 5′ to 3′) a glycosylphosphatidylinositol (GPI)-anchored membrane protein consisting of a SP, one or more unique polypeptides optionally connected by a spacer, and GPI anchor domain. In another embodiment, the mRNA CDS (150) comprises (from 5′ to 3′) a multi-pass membrane protein comprised of one or more unique polypeptides optionally connected by a spacer, and two or more transmembrane domains.

The mRNA CDS (150) can be configured to encode one or more covalently-linked unique polypeptides and comprises one or more spacers between each unique antigen protein. For example, a concatenated mRNA CDS (151) can be constructed that encodes for a polypeptide comprising a signal peptide (SP), a first antigen protein (P.1) and a second antigen protein (P.2) separated by a spacer (S1). A concatenated mRNA CDS of various lengths (152) can be constructed that encodes for a polypeptide with a signal peptide (SP), a first antigen protein (P.1), a spacer (Si) between the first antigen protein (P.1) and each subsequent antigen protein (Pi), and a third or subsequent antigen protein (Pn+2), where each consecutive antigen protein is separated by a spacer (Sn). Another CDS (153) can be constructed that encodes for a polypeptide that comprises a signal peptide (SP), a first antigen protein (P.1), a second antigen protein (P.2), a third antigen protein (P.3), and a fourth antigen protein (P.4), wherein each antigen protein is separated by a spacer (S1), (S2) and (S3) respectively. The mRNA spacer CDSs can encode polypeptides comprised of G/P (gly-pro), G/S (gly-ser) or AAY that are positioned between concatenated antigen proteins. In some embodiments, the mRNA polynucleotide sequence of the CDS (150) further encodes (x) a LAMP-1 signal peptide and a LAMP-1 transmembrane and cytoplasmic domain; or (y) a human HLA Class I signal peptide and a LAMP-1 transmembrane and cytoplasmic domain. SEQ ID NO: 35 is the N-terminal signal peptide of human LAMP-1 (Uniprot ID P11279, amino acids 1-28); SEQ ID NO: 36 is the C-terminal domain of human LAMP-1, including the transmembrane and cytoplasmic domains.

The RNA according to aspects of the disclosure may have modified ribonucleotides in order to increase its stability and/or decrease cytotoxicity. For example, in some embodiments, 5-methylcytidine in the RNA is substituted partially or completely, for cytidine. In some embodiments, 5-methylcytidine in the RNA is substituted completely for cytidine. Alternatively or additionally, in some embodiments, pseudouridine in the RNA used is substituted partially or completely, for uridine. In some embodiments, pseudouridine in the RNA used is substituted completely for uridine. In some aspects, the LNP comprises nucleic acid comprising a 5′ untranslated region (UTR) and 3′ UTR, polyA tail of about 80 to about 140 nucleotides in length, and (i) a 5′ enzymatic or (ii) a 5′ clean cap. In some aspects, the LNP comprises a chemically modified mRNA, wherein the chemically modified mRNA comprises N1-methylpseudouridine. In other embodiments, the mRNA does not include N1-methyl-pseudouridine.

FIG. 23B is a schematic of a mRNA construct (200) comprising a 5′ cap, 5′ UTR, a polypeptide coding sequence (CDS), 3′ UTR, polyA tail. The CDS encodes apolypeptide antigen (P). A Type I single-pass membrane protein mRNA construct (210) encodes a signal peptide encoded at the 5′ end (N-terminus of the protein). The transmembrane Domain™ with or without a cytoplasmic tail (CT) is encoded at the 3′ end (C-terminus of the protein). Most Type I membrane proteins contain a cleavable SP, but it is not an absolute requirement. A Type II single-pass membrane protein mRNA construct (220) encodes a TM encoded at the 5′ end (N-terminus of the protein). A Type III single-pass membrane protein mRNA construct (230) encodes the N-terminus of the encoded polypeptide is on the extracellular side of the membrane, followed by a TM domain, followed by an intracellular portion at the C-terminus. There is nosignal peptide. A Glycosylphosphatidylinositol (GPI)-anchored protein mRNA construct (240) encodes a SP at the 5′ end (N-terminus), and a GPI-anchor sequence that attaches the polypeptide to a lipid bilayer membrane is located at the 3′ end of the CDS (C-terminus of the polypeptide). A multi-pass membrane protein mRNA construct (250) encodes a CDS containing multiple transmembrane domains. A secreted protein mRNA construct (260) encodes signal peptide is encoded at the 5′ end (N-terminus of the protein) and there is no transmembrane domain. An example of a secreted antigen mRNA construct (270) consists of two concatenated polypeptides. A CDS encoding a polypeptide comprising an N-terminal SP and two unique polypeptides connected with a short polypeptide spacer. An example of a type I membrane protein mRNA construct (280) contains two concatenated polypeptides. A CDS encoding a polypeptide comprising an N-terminal SP, two unique polypeptides connected with a short polypeptide spacer, and a C-terminal TM domain.

3f. LNP Compositions

Unless explicitly indicated otherwise, the isomer form of the phosphatidylserine (PS) lipids used in the Examples is phosphatidyl-L-serine. In some aspects, the LNP composition is further characterized in that: the nucleic acid is mRNA; the ionizable cationic lipid is present in the LNP composition at a N/P ratio of 4 to 7 relative to the nucleic acid; the one or more phospholipids in the LNP comprise a phosphatidylserine (PS) lipid in a total amount of 2.5-10 mol % of the total lipid content of the LNP composition; and the conjugated lipid is a PEG-containing conjugated lipid. In some embodiments, the LNP is a vaccine composition comprising lipid nanoparticles encapsulating mRNA encoding a vaccine antigen (e.g., Tables 5A and 5B), and formed from (x) the ionizable lipid of Formula (I) (e.g., 45-55 mol % KC3-X1-PH5, KC3-X1-PH12, or KC3-X2-PH12), (y) the sterol (e.g., 25-45 mol % cholesterol), (c) a phosphocholine phospholipid (e.g., 1,2-distearoyl-sn-glycero-3-phosphocholine (DSPC), hydrogenated soy phosphatidylcholine (HSPC), or egg sphingomyelin (SM)), (d) a phosphatidyl-L-serine or phosphatidylglycerol phospholipid selected to target dendritic cells (e.g., 2.5-10 mol %, 2.5-7.5 mol %, or 5 mol % dipalmitoylphosphatidyl-L-serine (DPPS), distearoylphosphatidyl-L-serine (DSPS) or (1,2-Distearoyl-sn-glycero-3-phosphoglycerol (DSPG)), and (e) a PEG-conjugated lipid (e.g., 0.5-4.0 mol %, 1.0-4.0 mol %, 1.5-4.0 mol %, 1.5-2.5 mol %, 0.5 mol %, 1.0 mol %, 1.5 mol %, 2.0 mol %, or 2.5 mol % PEG(2000)-dimyristoylglycerol (PEG-DMG) or 1,2-dimyristoyl-rac-glycero-3-methoxypolyethylene glycol-2000 (PEG-DLG)).

In some embodiments, the LNP composition is formulated as a vaccine composition comprising a ionizable cationic lipid, one or more phospholipids, a sterol and a PEG-conjugated lipid, and wherein the vaccine composition comprises a nucleic acid sequence encoding an epitope or other antigen encapsulated in lipid nanoparticles formed from: (a) an ionizable lipid comprising an ionizable cationic lipid at a N/P ratio of 4 to 6 relative to the encapsulated nucleic acid, the ionizable lipid present in the LNP composition in a total amount of 46-54 mol % of a total lipid content of the LNP composition; (b) one or more phospholipids comprising a neutral phospholipid such as distearoylphosphatidylcholine (DSPC), hydrogenated soy phosphatidylcholine (HSPC), dipalmitoylphosphatidylcholine (DPPC), or combination thereof, (c) one or more anionic phospholipids selected from a phosphatidylserine (PS) or a phosphatidylglycerol (PG) or a combination thereof, in a total amount of 2.5-10 mol % of the total lipid content of the LNP composition; and (d) a PEG(2000)-conjugated lipid in a total amount of 0.5-5.0 mol % of the total lipid content of the LNP composition.

In some embodiments, the LNP composition is formulated as a vaccine composition comprising a ionizable cationic lipid, one or more phospholipids, a sterol and a PEG-conjugated lipid, and wherein the vaccine composition comprises a nucleic acid sequence encoding an epitope or other antigen encapsulated in lipid nanoparticles formed from: (a) an ionizable lipid comprising an ionizable cationic lipid at a N/P ratio of 4 to 6 relative to the encapsulated nucleic acid, the ionizable lipid present in the LNP composition in a total amount of 46-54 mol % of a total lipid content of the LNP composition; (b) one or more phospholipids comprising a neutral phospholipid selected from distearoylphosphatidylcholine (DSPC), hydrogenated soy phosphatidylcholine (HSPC), dipalmitoylphosphatidylcholine (DPPC), or combination thereof, in a total amount of 5 mol % of the total lipid content of the LNP composition and (c) one or more anionic phospholipids selected from a phosphatidylserine (PS) or a phosphatidylglycerol (PG) or a combination thereof, in a total amount of 5 mol % of the total lipid content of the LNP composition; and (d) a PEG(2000)-conjugated lipid in a total amount of 0.5-5.0 mol % of the total lipid content of the LNP composition.

Additional formulations include compositions in the Table of Example 18. All lipid concentrations are shown as mol % as a percentage of the total lipid in the LNP. In some embodiments, the LNP compositions can vary in conjugated lipid content from 1.5 to 4 mol %, in sterol content from 23-43 mol %, in ICL content from 46-56 mol %, in anionic phospholipid (e.g., saturated phosphatidyl-L-serine or phosphatidylglycerol) content from 2.5-7.5 mol %, and in total noncationic phospholipid content from 2.5-15 mol %, with a total of 10-15 mol % of phospholipids (anionic and non-cationic phospholipids combined). In some embodiments, the LNP composition contain two phospholipids totaling 10 mol %, typically an anionic phospholipid from 2.5-7.5 mol % (e.g., a phosphatidyl-L-serine—DSPS or DPPS being preferred—or phosphatidylglycerol (e.g. DSPG) and 2.5-7.5 mol % a neutral phosphatidylcholine phospholipid, although some exemplary formulations may contain more than two phospholipids. LNPs may contain mRNA or other nucleic acid at N/P of 5-7 (e.g., 5.25). The ionizable cationic lipid can be any ionizable lipid described herein, including KC3-X1-PH5, KC3-X1-PH12, or KC3-X2-PH12.

4. ICL Compounds for Enhancing mRNA Expression in LNP Compositions

In some aspects, the present disclosure is based, in part, on the discovery that mRNA expression from lipid nanoparticle (LNP) formulations unexpectedly depends on the structure of hydrolysable or bioreducible ionizable cationic lipids (ICLs) within the LNP. Specifically, LNPs containing hydrolysable ICLs of Formula (I) or (I-A) exhibit mRNA transfection activity that is strongly influenced by the structure of the functionalized aliphatic ICL tails, an effect that can be approximated by linear relationships. Unexpectedly, the mRNA transfection activity of the LNP compositions with hydrolysable ICLs of Formula (I) and (I-A) showed strong dependence on the structure of the ICL that could be approximated by linear trendlines. For example, the mRNA transfection activity of tested formulations (Example 7 and Example 8) showed strong dependence on the ratio of the number of carbons (x) in X1 and X2 in Formula (I-A) to the total number of carbon atoms in X1+Y1 and X2+Y2 [(x)+(y)], respectively, which was approximated by a linear trendline, with ICLs with larger number of carbons X1+Y1 and X2+Y2, of Formula (I-A) having higher activity than the ones with smaller number of carbons in X1+Y1 and X2+Y2, of Formula (I-A) for any given number of X1 and X2, respectively.

For example, luciferase (Luc) mRNA transfection activity from LNP formulations with hydrolysable ionizable cationic lipids of Formula (I) or (I-A) unexpectedly varied depending on the ratio of carbon atoms in X1 and X2 compared to the Y1 and Y2 regions of ICLs of Formula (I-A). FIG. 24 shows linear trends in the Luc mRNA transfection activity as a function of the structure of ICLs (structure of X1/X2 and Y1/Y2 in Formula (I)), for ICL compounds where X1 and X2 are identical and Y1 and Y2 are identical. In FIG. 24, Cn refers to the number of carbons (x) in each of X1 and X2, and Cm refers to the total number of carbons (y) in each of Y1 and Y2.

FIG. 25 shows linear trends in the minimum levels of carbon atoms in the Y portion of an ICL molecule (X1/X2 and Y1/Y2 in Formula (I-A)) (y) to achieve 10%, 50% or 100% luciferase expression activity of KC3-OA-LNP, calculated from the linear approximations shown on FIG. 24, and plotted against various numbers of carbon atoms between the ester group and the dioxolane (ketal) ring (n).

The mRNA transfection activity of tested formulations showed dependence on the of the number of carbons (x) in each of X1 and X2 in Formula (I). The luciferase (Luc) mRNA transfection activity from LNP formulations with hydrolysable ionizable cationic lipids of Formula (I) unexpectedly varied depending on the ratio of carbon atoms in X (x) compared to the Y (y) regions of ICLs of Formula (I) with greater activity observed for ionizable cationic lipids of Formula (I) where the number of carbons (x) in each of X1 and X2 is greater than 5 (e.g., 6-10). By comparison, minimal expression (e.g., below 5% of the Luc activity of the KC3-OA reference) was observed in ionizable cationic lipids of Formula (I) with (x) in each of X1 and X2 less than 6. FIG. 25 shows the Luc mRNA transfection activity as a function of the structure of ICLs of Formula (I) based on the number of carbons (x) in X1 and X2.

Aspects of the disclosure are based in part on the discovery that expression of mRNA in certain hydrolyzing LNP formulations unexpectedly depends on the structure of hydrolysable or bioreducible ionizable cationic lipid components in LNP formulations comprising mRNA. Unexpectedly, the mRNA transfection activity of the LNP compositions with hydrolysable ICLs of Formula (I) showed strong dependence on the structure of the pair of aliphatic linear hydrocarbon chains flanking a bioreducible and/or hydrolysable functional group. The mRNA transfection activity surprisingly varied as a function of the aliphatic hydrocarbon chain structures, as shown in the data of FIG. 24 and Table A-1.

TABLE A-1

| Cx | Cy | Luc, % of KC3-OA |
|---|---|---|
| 1 | 17-18 | 0.4-0.5 |
| 3 | 13 | 0.0-0.2 |
| 4 | 12-16 | 0.0-4.6 |
| 5 | 11-15 | 0.0-2.5 |
| 6 | 12-14 | 36.2-61.8 |
| 7 | 9 | 2.9-27.4 |
| 7 | 11-13 | 34.6-200.4 |
| 8 | 2 | 0.7 |
| 8 | 10-14 | 38.0-227.0 |
| 9 | 11-12 | 117.9-229.3 |
| 10 | 10-12 | 79.8-199.1 |

These discoveries collectively demonstrate that the chemical structure of hydrolysable or bioreducible ICLs can be systematically tuned to optimize mRNA expression in LNP formulations, providing a framework for designing more potent and effective mRNA-LNP therapeutics.

### 5. Immunogenic LNP Compositions

The immunogenicity and functional performance of various lipid nanoparticle (LNP) vaccine formulations encapsulating mRNA encoding pathogen-specific antigens was evaluated. Across multiple disease models, the experiments were designed to characterize both cellular immune responses (T cell activation and cytokine production) and humoral responses (antibody titers), as well as early innate immune activation reflected in circulating cytokines and chemokines.

Together, these measurements provide a comprehensive view of how effectively each LNP formulation induces antigen-specific immunity, supports immune memory, and engages pathways known to correlate with protection against infectious disease.

The measurements span several complementary immunological readouts. Percent cytokine-positive CD4⁺ and CD8⁺ T cells quantify the fraction of helper and cytotoxic T cells producing effector cytokines (e.g., IFN-γ, TNF-α, IL-2) after antigen stimulation, serving as indicators of antigen-specific cellular immunity. Endpoint titers (e.g., SD21, SD28, SD34, SD35, SD56) reflect the magnitude of antigen-specific antibody responses, typically defined as the highest serum dilution at which antibodies remain detectable; these are key correlates of humoral immunity and protection for many viral pathogens. Timepoint-specific measurements (e.g., SD21, SD49) track the kinetics and durability of immune responses following vaccination. In addition, circulating cytokines and chemokines—including Interferon gamma, Interleukin 6, Tumor necrosis factor alpha, Interferon alpha, Interleukin 12, CCL2, CCL5, Interleukin 1 beta, CXCL1, and CXCL10—provide insight into innate immune activation, inflammation, and immune cell recruitment shortly after vaccination, all of which influence downstream adaptive responses.

In addition to humoral immunity, evaluating CD4⁺ and CD8⁺ T cell responses is essential for understanding the full immunological impact of LNP-encapsulated mRNA vaccines encoding an H5 antigen. CD4⁺ T helper cells support the development of high-affinity, class-switched IgG antibodies and durable immune memory through their role in germinal center formation and B cell maturation. Concurrently, CD8⁺ cytotoxic T lymphocytes contribute to cellular immunity by recognizing and eliminating antigen-expressing cells, which is particularly important for protection against intracellular pathogens such as influenza viruses. Robust CD4⁺ and CD8⁺ responses indicate efficient antigen processing and presentation via both MHC class II and class I pathways, reflecting effective mRNA translation and innate immune activation by the LNP formulation. Together, these T cell responses complement antibody measurements by providing insight into the breadth, quality, and durability of vaccine-induced immunity.

Mycobacterium tuberculosis Studies

In Example 10, LNP formulations containing different ionizable cationic lipids (ICLs) were evaluated using an mRNA encoding a synthetic Mycobacterium tuberculosis vaccine antigen, with immune responses assessed solely by the percentage of cytokine-positive CD4⁺ and CD8⁺ T cells. These measurements provide a direct readout of antigen-specific cellular immunity, which is critical for protection against M. tuberculosis, as Th1-type CD4⁺ T cells activate infected macrophages and cytotoxic CD8⁺ T cells contribute to bacterial clearance. By focusing on cytokine-producing T cells, this study highlights how different LNP compositions influence the magnitude and quality of the functional cellular immune response, providing key insight into the potential efficacy of each formulation for tuberculosis vaccination. For the tuberculosis antigen studies (Example 10), the primary focus is on cellular immunity, as protection against Mycobacterium tuberculosis relies heavily on T cell-mediated responses rather than antibodies alone. Measurements of cytokine-producing CD4⁺ T cells are particularly important because Th1-type CD4⁺ responses (e.g., IFN-γ production) are critical for activating infected macrophages and controlling intracellular bacterial growth. CD8⁺ T cell cytokine responses also contribute by supporting cytotoxic activity and enhancing bacterial clearance. Thus, the percentage of cytokine-positive T cells provides a functional readout of vaccine-induced immunity and is directly relevant to predicting protective efficacy in tuberculosis.

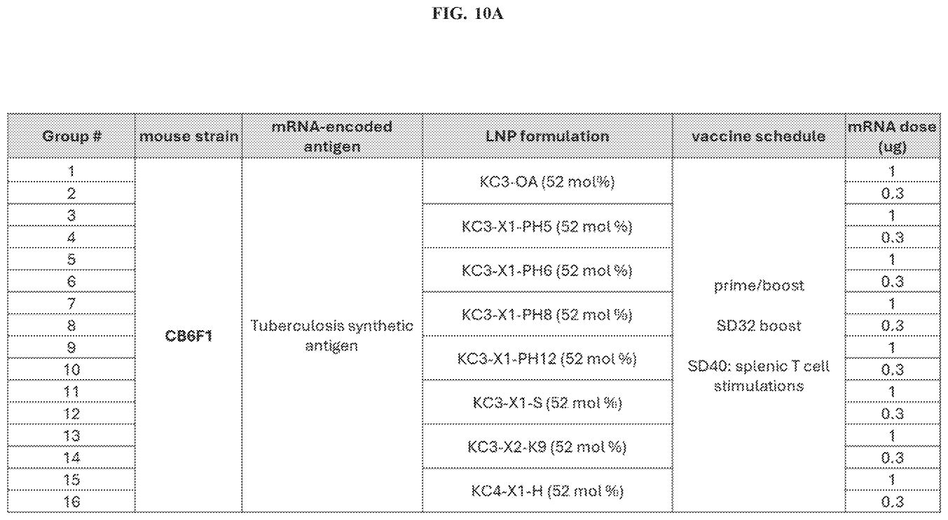

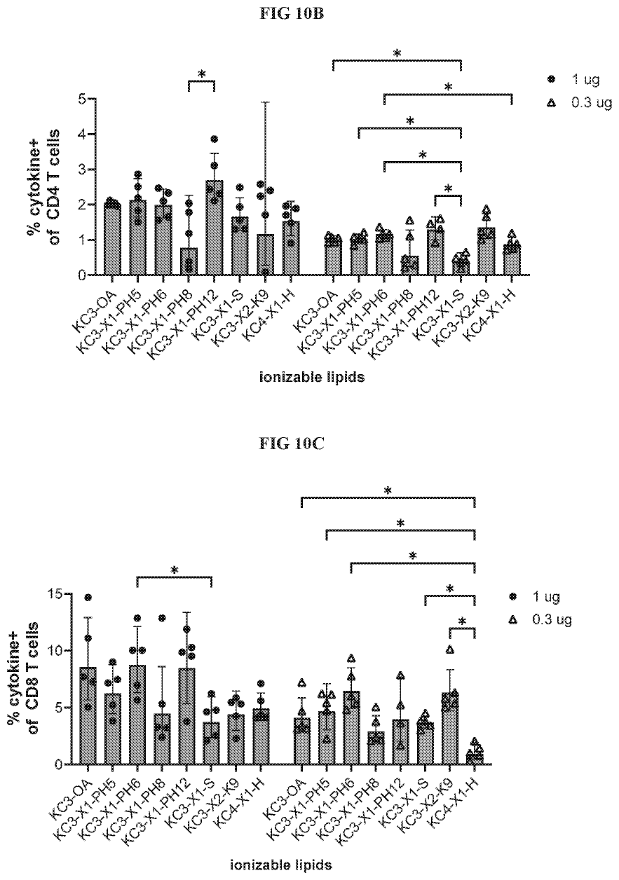

In Example 10A (FIGS. 10A-10C), mice were injected with 1 or 0.3 μg mRNA of LNP with different ICL compounds and given a homologous boost on study day (SD) 32 (FIG. 10A). The resulting magnitudes of the CD4 T cell (FIG. 10B) and CD8 T cell (FIG. 10C) TB-specific responses were generally comparable between LNP formulations at both the 1 and 0.3 μg dose, but there were some formulations that elicited smaller responses. For the CD4 responses, the ICLs KC3-X1-PH8 and KC3-X1-S were inferior relative to other formulations, most notable KC3-X1-PH12. For the CD8 responses, KC3-X1-PH8 trended smaller relative to the other formulations, and at the 0.3 μg dose, KC4-X1-H was inferior to most of the other ICLs. In conclusion, all of these mRNA-LNPs formulated with these biodegradable ICLs were capable of eliciting antigen-specific T cell immunity at the doses tested. All except for KC3-X1-PH8 and KC3-X1-S performed comparably to KC3-OA.

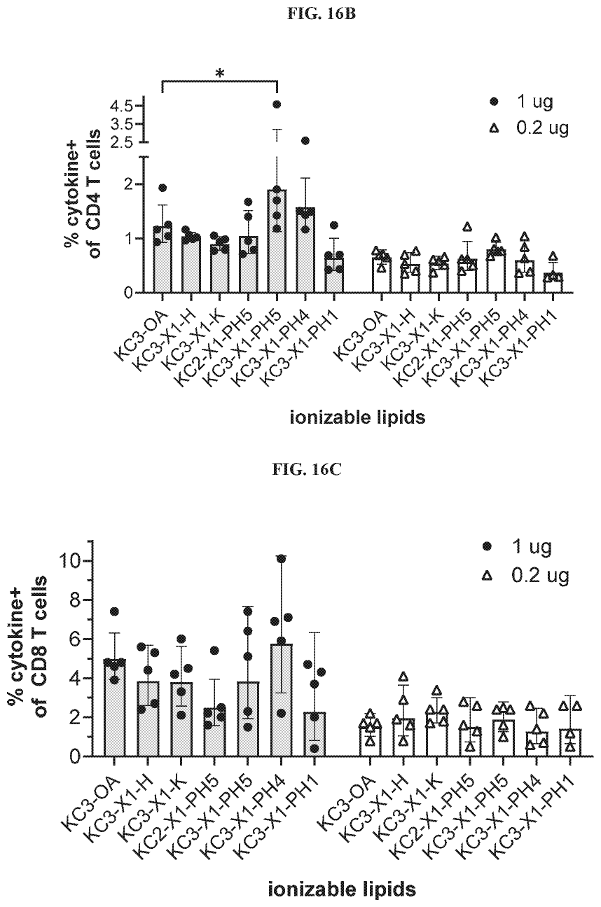

In Example 10B, the immunogenicity of mRNA-LNPs encapsulating mRNA encoding a Mycobacterium tuberculosis synthetic antigen in LNPs formed from various ICL compounds, and were injected in mice to evaluate T-cell responses (study design in FIG. 16A). The magnitudes of the CD4 T cell (FIG. 16B) and CD8 T cell (FIG. 16C) TB-specific responses were comparable between LNP formulations at both the 1 and 0.2 μg dose. Only the CD4 T cell response elicited by the KC3-X1-PH5 formulation was significantly greater than KC3-OA. Thus, LNPs comprised of these biodegradable ICLs were capable of eliciting robust antigen-specific T cell immunity at the doses tested.

In Example 10C, mRNA-LNPs having varying the mol % of the ICL designated KC3-X1-PH12 LNPs were injected in mice to evaluate how changing the amount of the ICL altered the activation and expansion of M. tuberculosis-specific T cells. The study design is shown in FIG. 21A. As expected, the magnitude of TB-specific CD4 cells decreased as the dose decreased due to limiting amounts of available antigen (compare FIGS. 21B-21D). For the CD8 T cell response, the size also increased with the mol % (FIG. 21E) but this was not statistically significant. The response magnitude at the 0.3 mcg dose was similar to that of the 1 mcg dose, and then it decreased at the 0.1 mcg dose. Importantly, the immunogenicity of the lyophilized and reconstituted formulations (Groups 9-12) was comparable to the non-lyophilized formulations. In conclusion, these data indicate that KC3-X1-PH12 at either 52 or 56 mol % is more optimal than 48 mol %. Stability is a critical consideration for vaccines, and we demonstrated here that lyophilized test articles did not decrease in immunogenicity despite increasing in size (Z-ave) and decreasing in encapsulation efficiency. In mice vaccinated with quadrivalent mRNAs, the primary question we tested here was whether vaccination with additional mRNAs would adversely affect the immunogenicity of the 2^{nd} gen mRNA. Shown in FIGS. 21H and 21I, it appears that the co-delivery of the 3 additional mRNAs potentially decreased the size of the CD4 T cell response to the 2^{nd} generation antigen; however it did not have an adverse effect on the CD8 response. In conclusion, the KC3-X1-PH12 LNP formulation is able to co-deliver multivalent vaccine mRNAs.

### Influenza Studies

A series of influenza studies (Example 11) were designed to evaluate how different ionizable cationic lipids within LNP formulations influence the magnitude and quality of immune responses to mRNA-encoded viral antigens. LNP-encapsulated mRNA encoding an influenza A H5 hemagglutinin antigen was used as a model system to track both B-cell (antibody titers over time) and T-cell responses (cytokine-producing CD4⁺ and CD8⁺ T cells), enabling assessment of both humoral and cellular immunity elicited by each formulation. This approach provides insight into the kinetics, durability, and functional quality of the immune response induced by distinct lipid compositions.

Lipid nanoparticle (LNP) compositions comprising mRNA encoding influenza A H5 antigen encapsulated in lipid nanoparticles comprising hydrolysable ionizable lipids of Formula (I) induced B-cell and T-cell responses at two different concentrations in murine models (Example 11D). Measuring H5-binding IgG antibody titers in mice three weeks after injection of LNP-encapsulated mRNA encoding an H5 antigen is an indicator of vaccine immunogenicity and functional antigen expression in vivo. This time point corresponds to the peak of the primary adaptive immune response, when class-switched IgG antibodies have been robustly generated following antigen presentation and T cell help. Quantification of H5-specific IgG titers therefore provides direct evidence that the mRNA was efficiently delivered, translated into properly folded antigen, and recognized by the immune system. Moreover, the magnitude of the H5-binding IgG response enables comparative assessment of different LNP formulations or mRNA constructs, linking delivery efficiency and antigen expression to downstream humoral immunity. As such, H5-specific IgG titers serve as a key correlate of immunogenic performance for mRNA-LNP vaccine candidates.

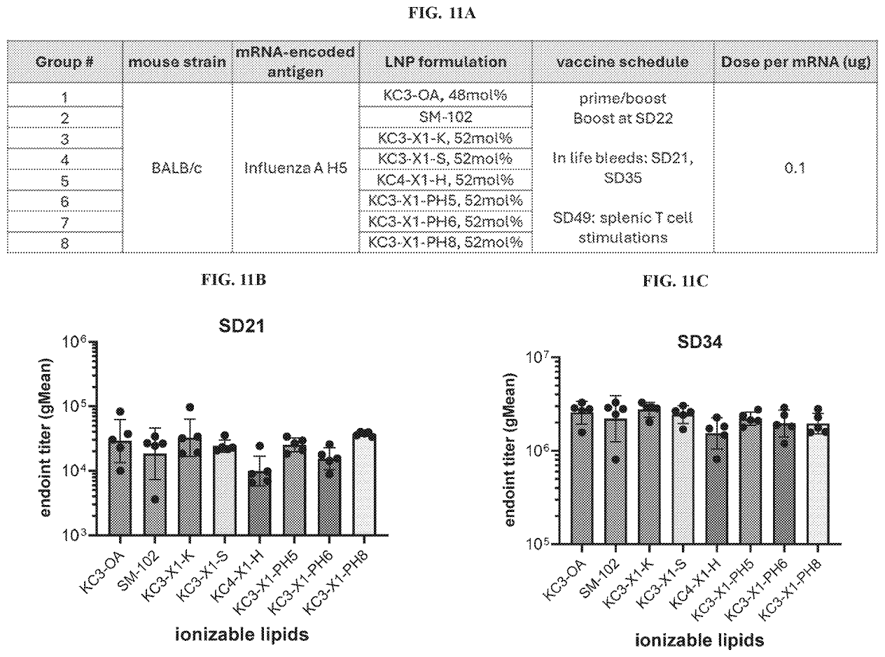

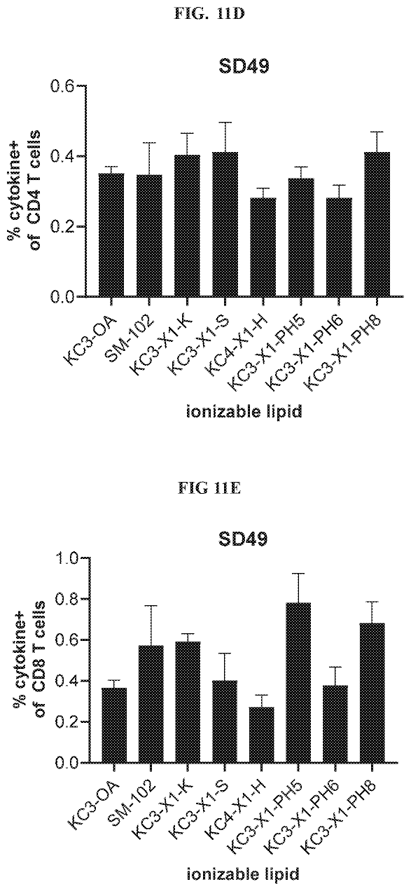

In Example 11A, the immunogenicity of mRNA encoding an influenza A H5 antigen was encapsulated in various LNPs comprising different ICLs was evaluated in mice by measuring B-cell and T-cell responses (FIG. 11A). HA-binding IgG titers on SD21 (FIG. 11B) and SD34 (13 days post-boost; FIG. 11C) were similar across groups. H5-specific CD4 (FIG. 11D) and CD8 T cell (FIG. 11E) memory pools were similar in magnitudes across ICLs. KC4-X1-H trended lower in all readouts, but this was not statistically significant relative to other groups. In conclusion, the ionizable lipids KC3-X1-K, KC3-X1-S, KC4-X1-H, KC3-X1-PH5, KC3-X1-PH6 and KC3-X1-PH8 readily formed into LNPs and had similar activity in vivo compared to the KC3-OA and commercial SM-102 formulations.

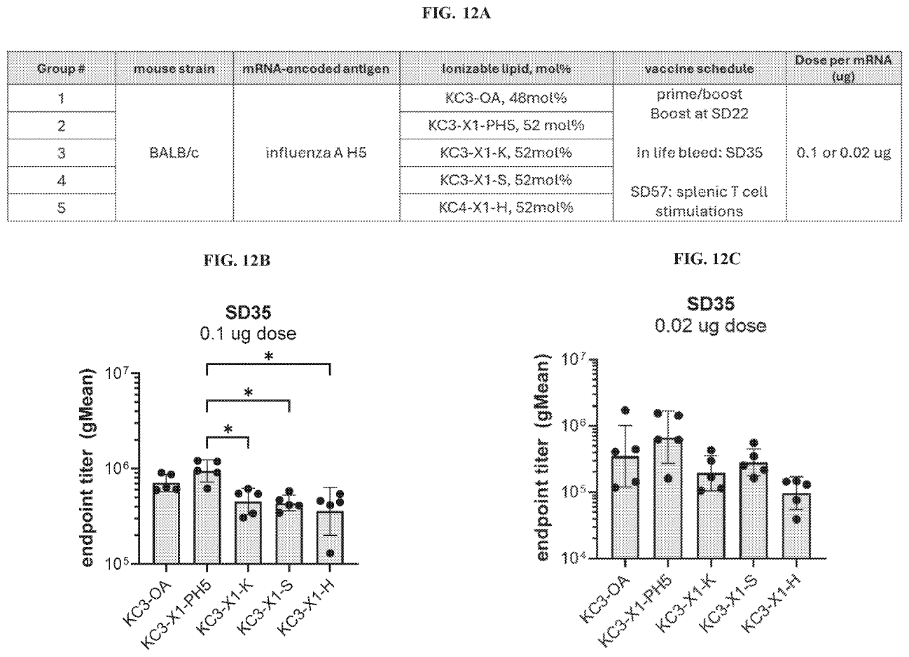

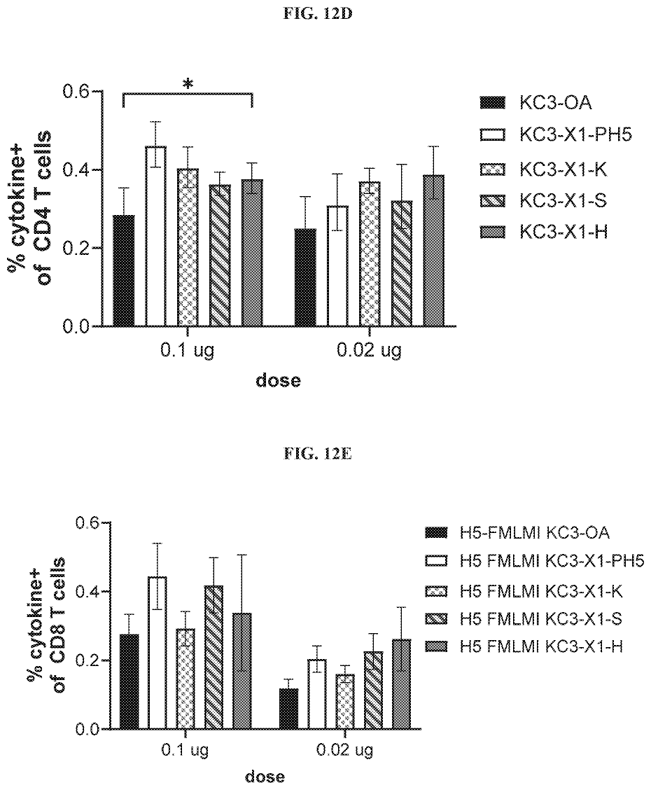

In Example 111B, the immunogenicity of mRNA encoding an influenza A H5 antigen was encapsulated in various LNPs comprising different ICLs was evaluated in mice by measuring B-cell and T-cell responses (FIG. 12A). For the 0.1 μg dose (FIG. 12B), titers for the KC3-X1-K, KC3-X1-S and KC3-X1-H groups were lower than KC3-X1-PH2. For the 0.02 μg dose (FIG. 12C), all groups had comparable titers. The size of the HA-specific CD4 T cell (FIG. 12D) and CD8 T cell responses (FIG. 12E) were comparable at both dose levels with the exception of the CD4 response for KC3-X1-H group at the 0.1 dose—it was significantly greater than that of the KC3-OA formulation. In conclusion, the novel lipids KC3-X1-PH5, KC3-X1-K, KC3-X1-S, KC4-X1-H had comparable activity to KC3-OA in eliciting antibody and T cell responses to a vaccine antigen.

In Example 11C (FIG. 21A), the magnitude of TB-specific CD4 cells decreased as the dose decreased due to limiting amounts of available antigen (compare FIGS. 21B-21D). For the CD8 T cell response, the size also increased with the mol % (FIG. 21E) but this was not statistically significant. The response magnitude at the 0.3 mcg dose was similar to that of the 1 mcg dose, and then it decreased at the 0.1 mcg dose. Importantly, the immunogenicity of the lyophilized and reconstituted formulations (Groups 9-12) was comparable to the non-lyophilized formulations. In conclusion, these data indicate that KC3-X1-PH12 at either 52 or 56 mol % is more optimal than 48 mol %. Stability is a critical consideration for vaccines, and we demonstrated here that lyophilized test articles did not decrease in immunogenicity despite increasing in size (Z-ave) and decreasing in encapsulation efficiency.

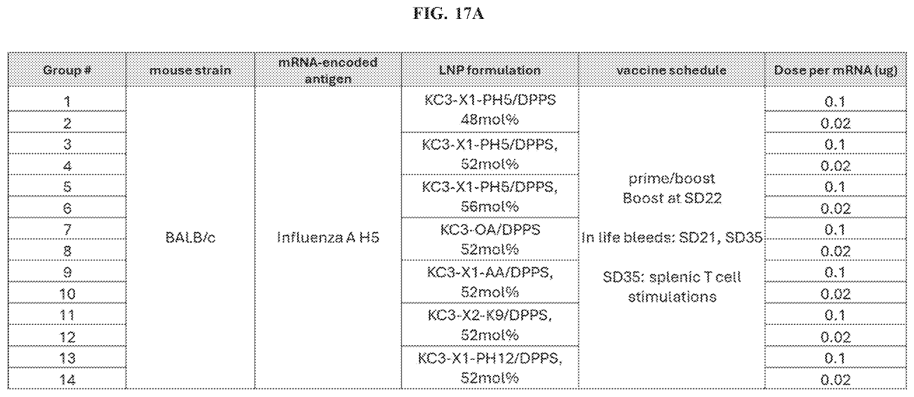

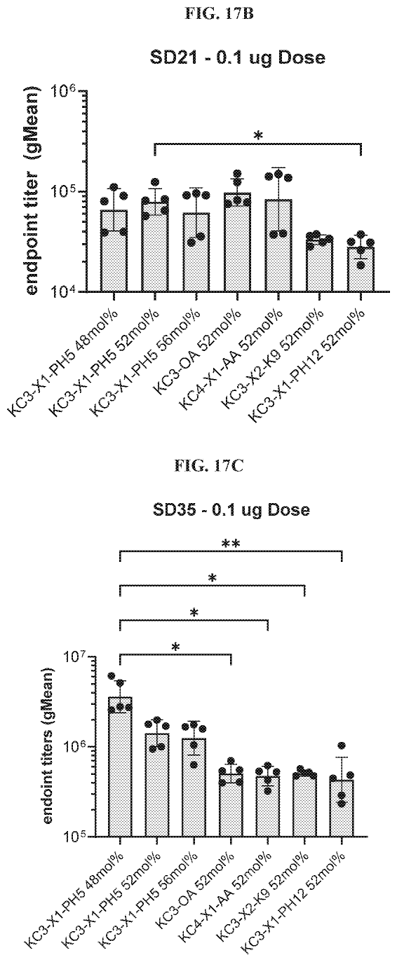

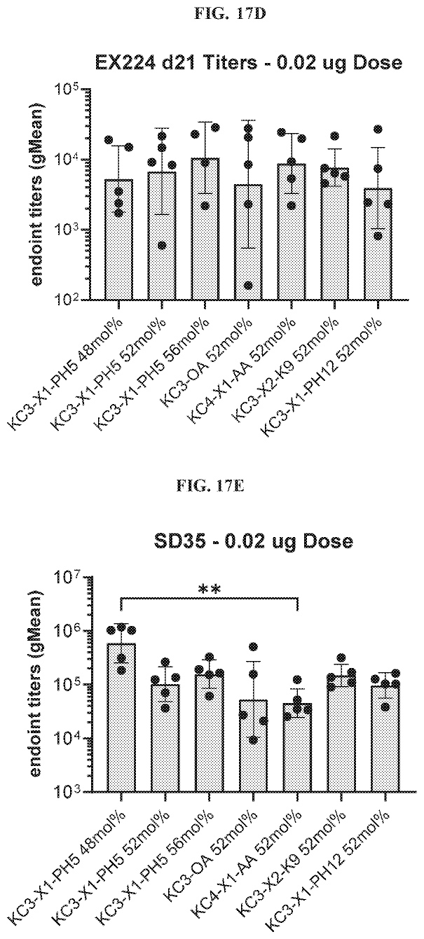

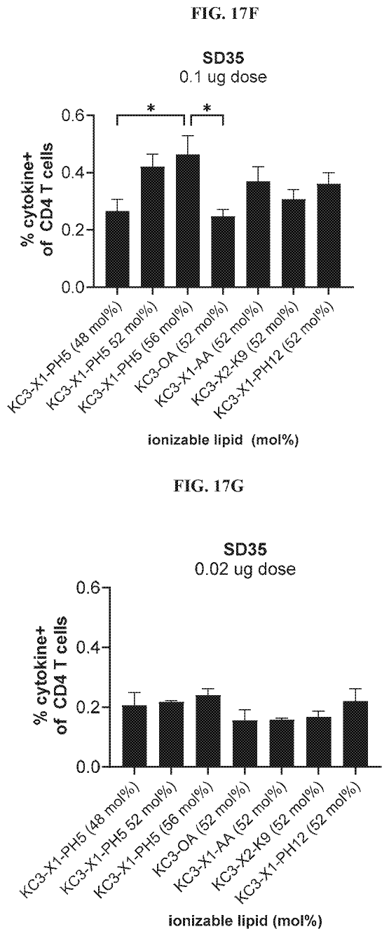

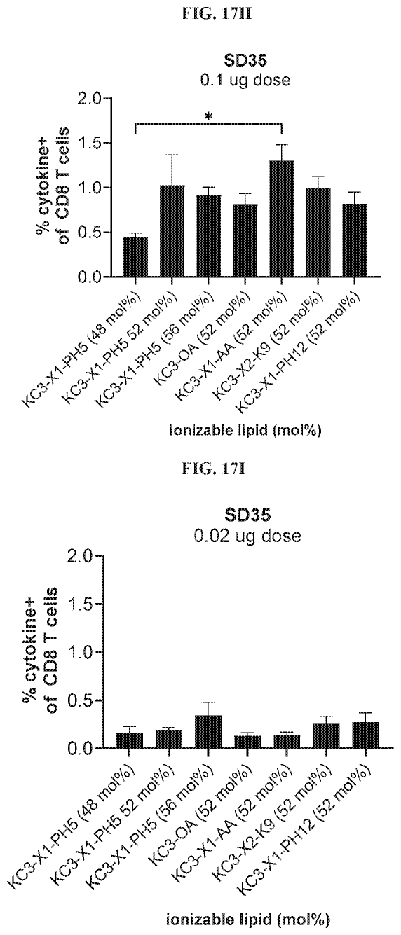

In Example 1 1D (FIG. 17A), total H5 binding IgG titers 3 weeks post primary vaccination were similar between the formulations at both doses with only KC3-X1-PH12 (52 mol %, 0.1 μg dose) being statistically lower than titers from the KC2-X1-PH5 52 mol % (FIG. 17B and FIG. 17D). After the 2^{nd} immunization, all titers were boosted. At the 0.1 μg dose (FIG. 17C), the KC3-X1-PH5 formulation (48 mol %) induced significantly higher titers than those of KC3-OA, KC3-X1-AA, KC2-X2-K9 and KC3-X1-PH12. At the 0.02 μg dose (FIG. 17E), KC3-X1-PH5 (48 mol %) was only higher than the KC3-X1-AA group. In conclusion, formulations containing the novel biodegradable ICLs elicited H5 binding IgG titers equivalent to KC3-OA. The data indicate that KC3-X1-PH5 is optimally formulated into LNPs at 48 mol %, although this was not statistically higher than the 52 and 56 mol % LNPs.

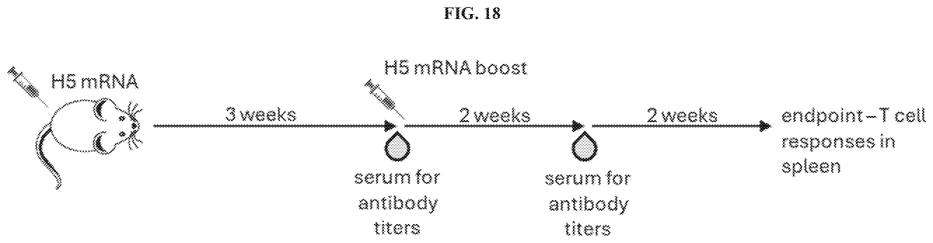

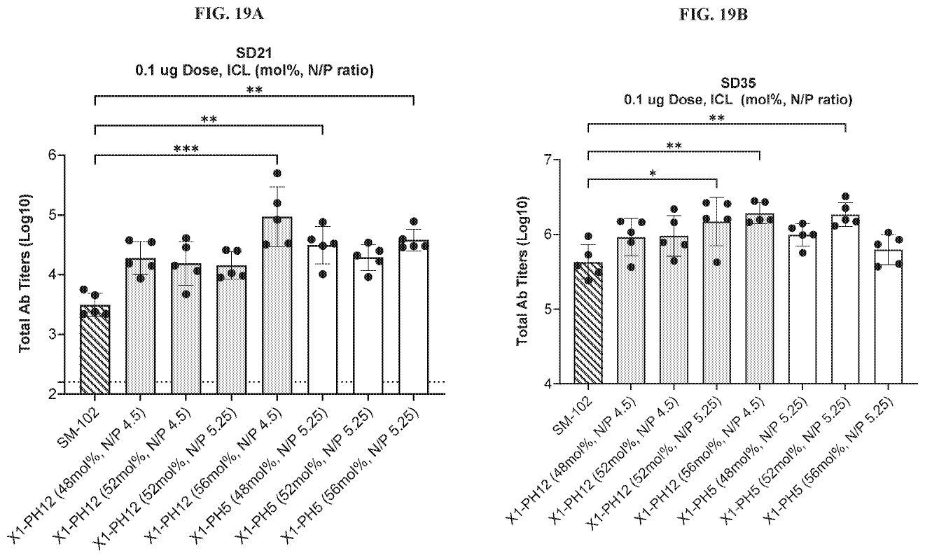

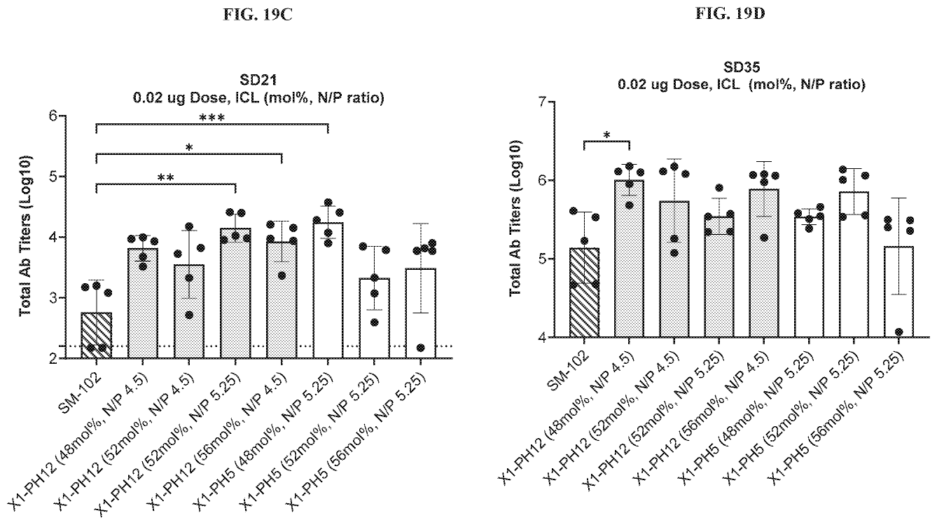

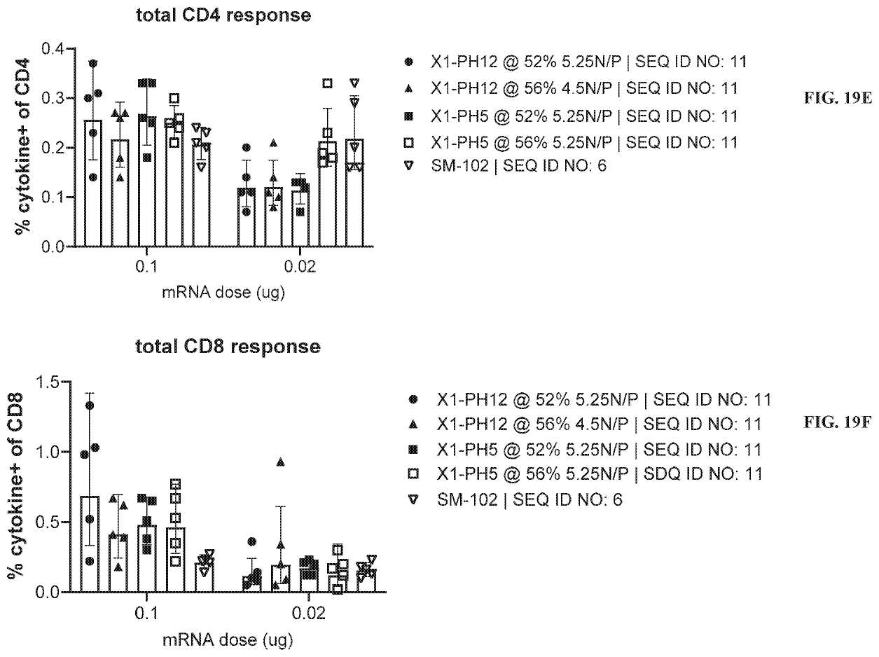

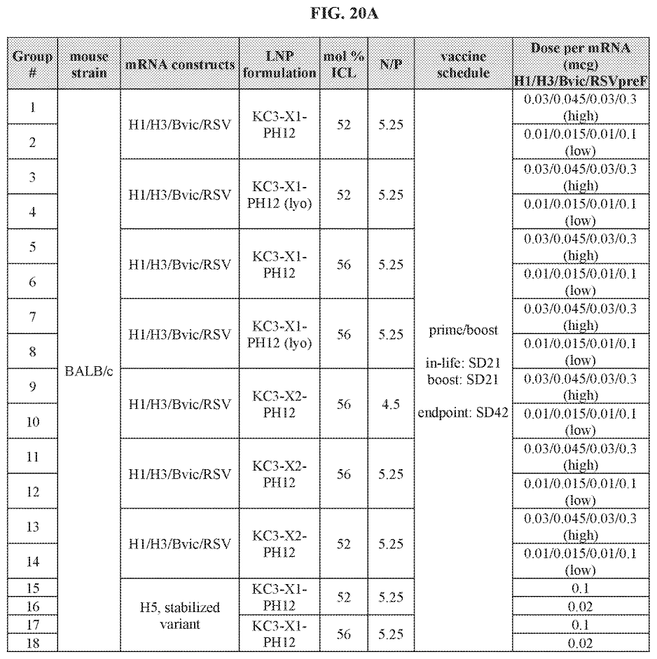

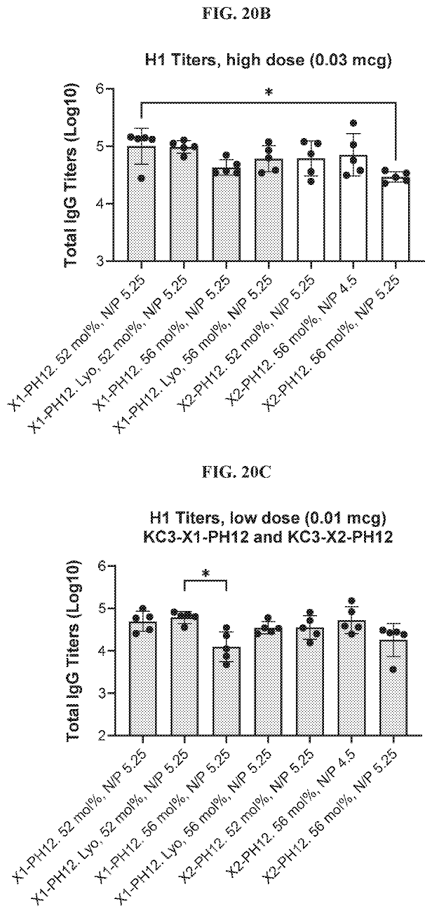

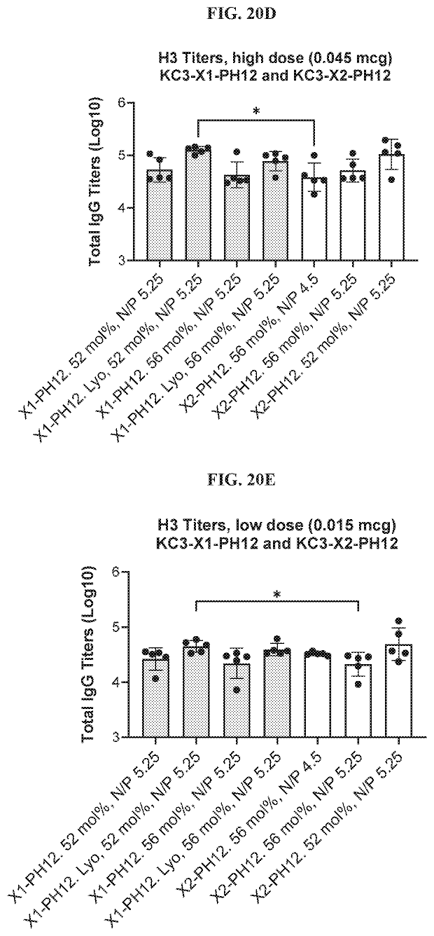

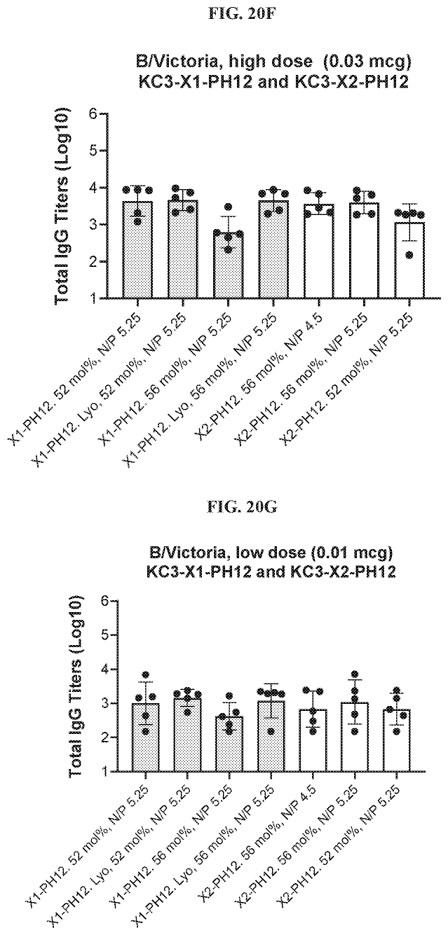

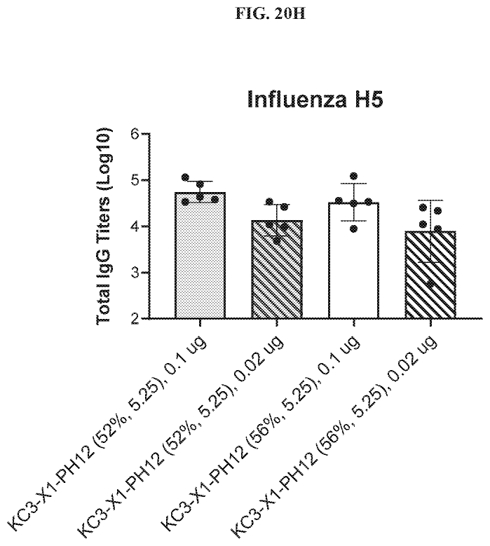

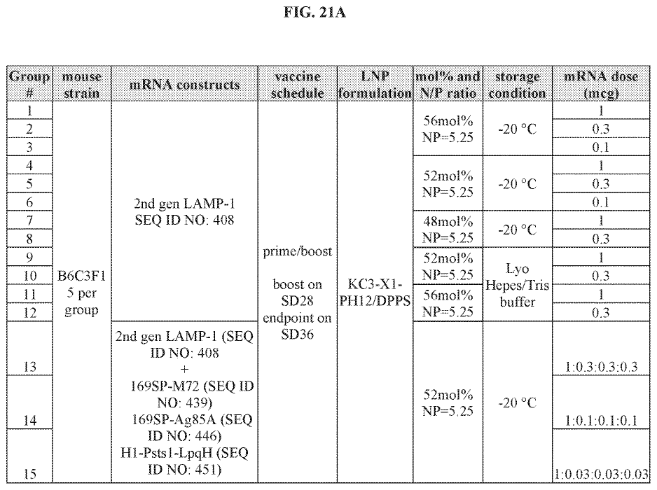

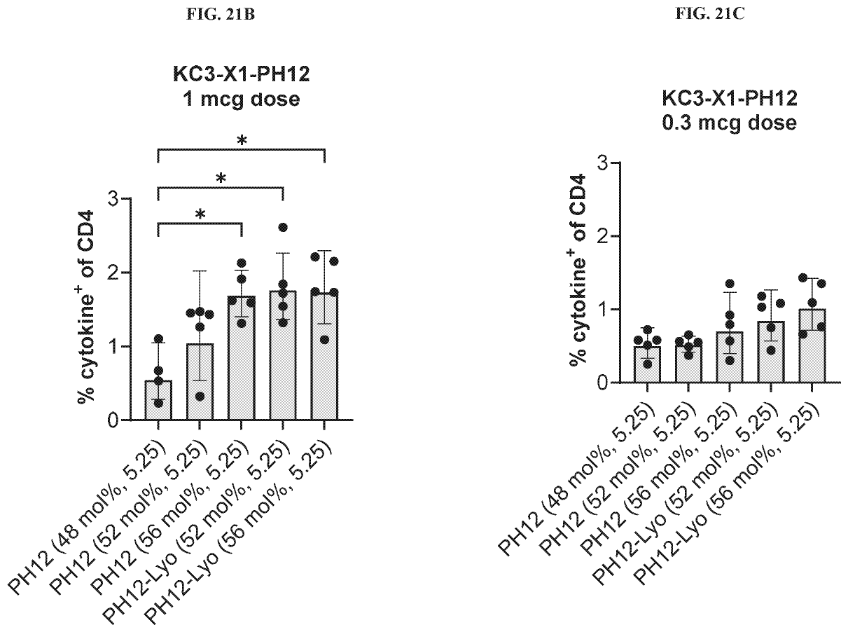

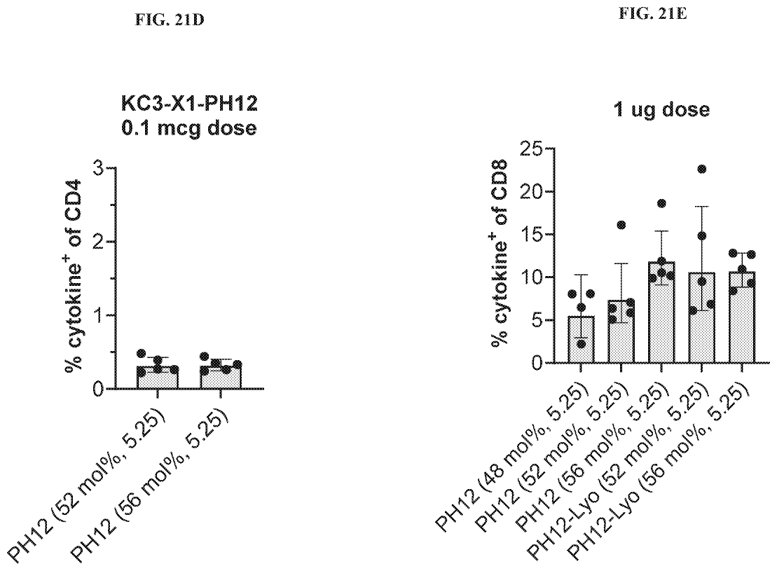

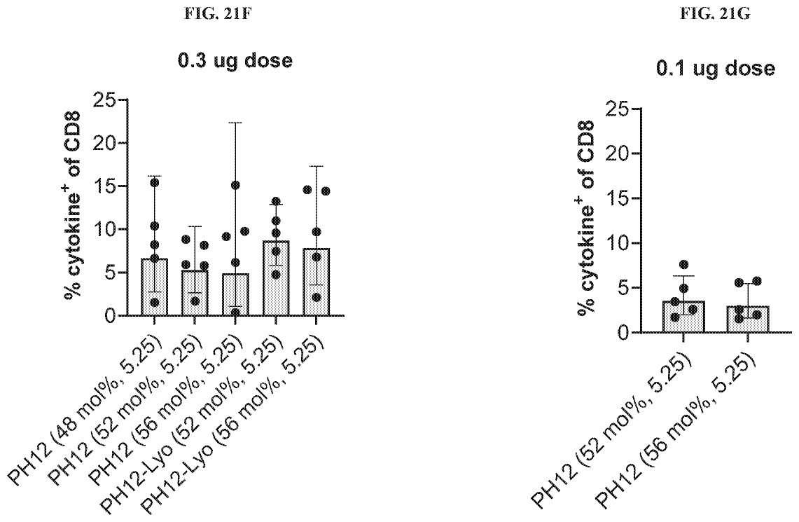

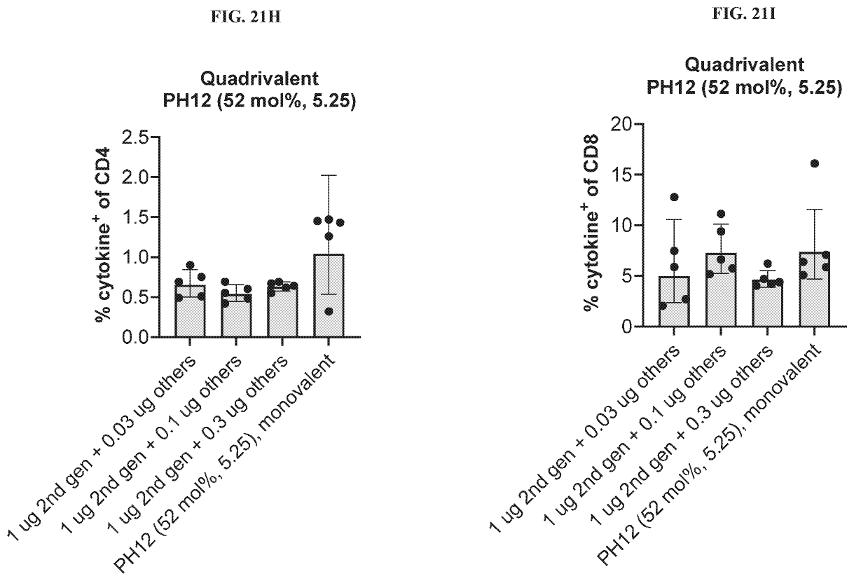

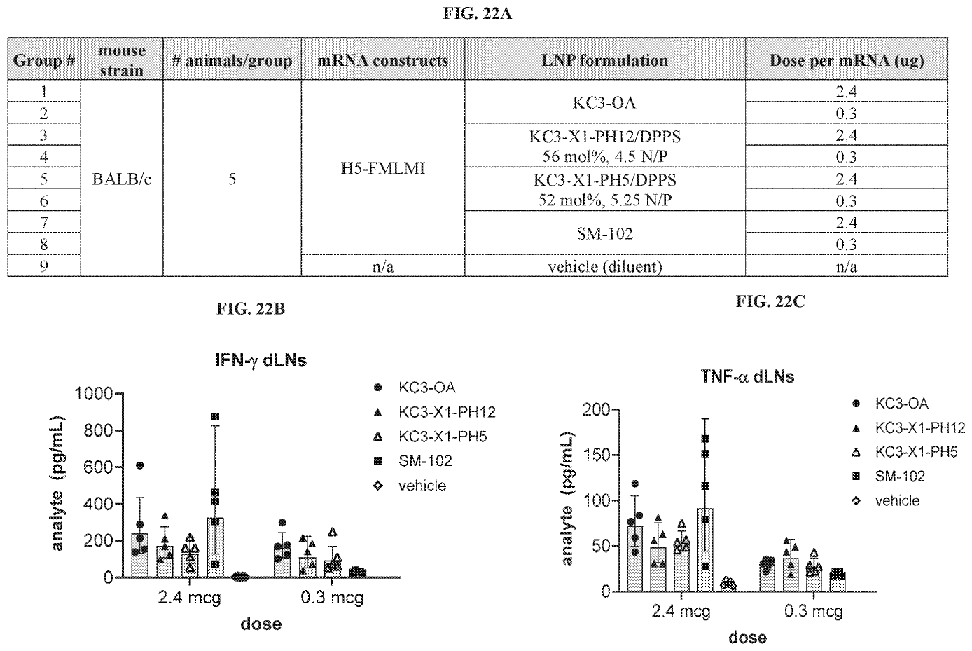

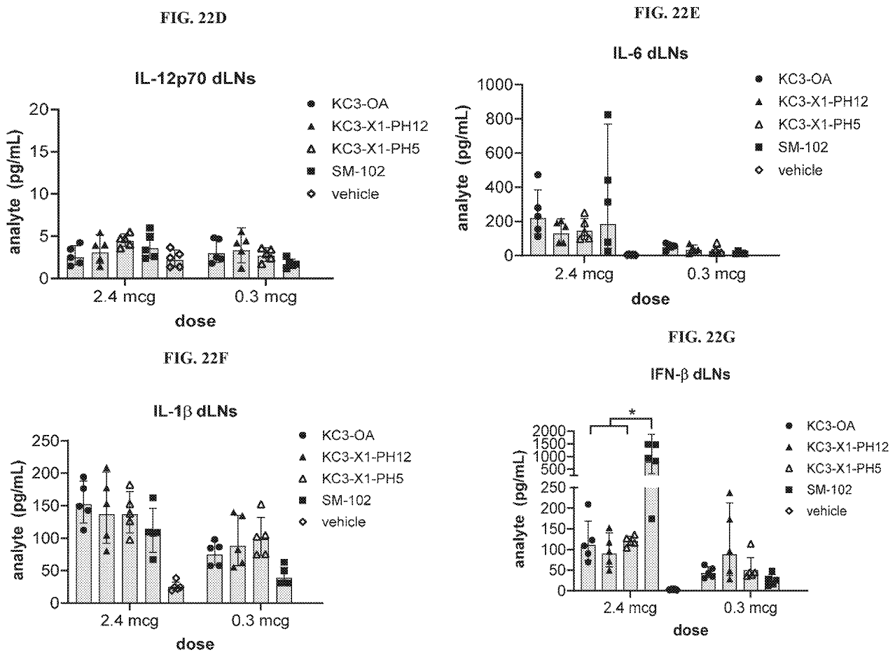

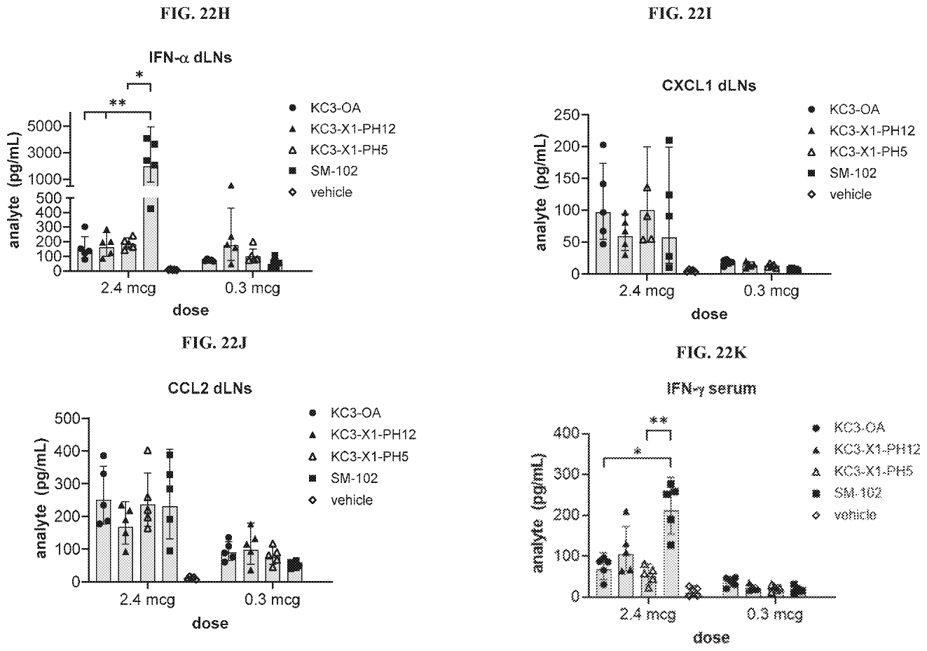

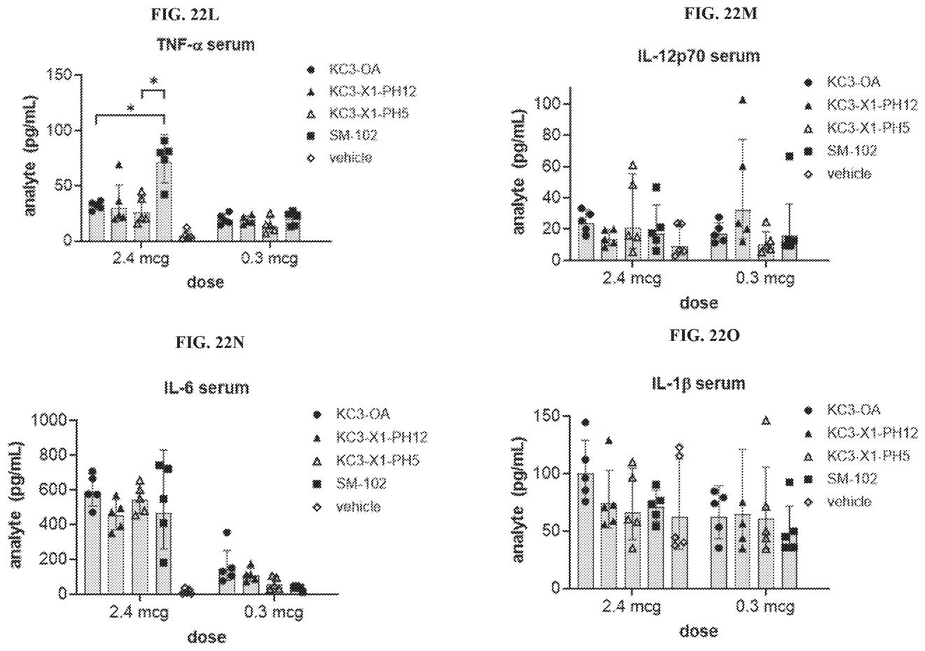

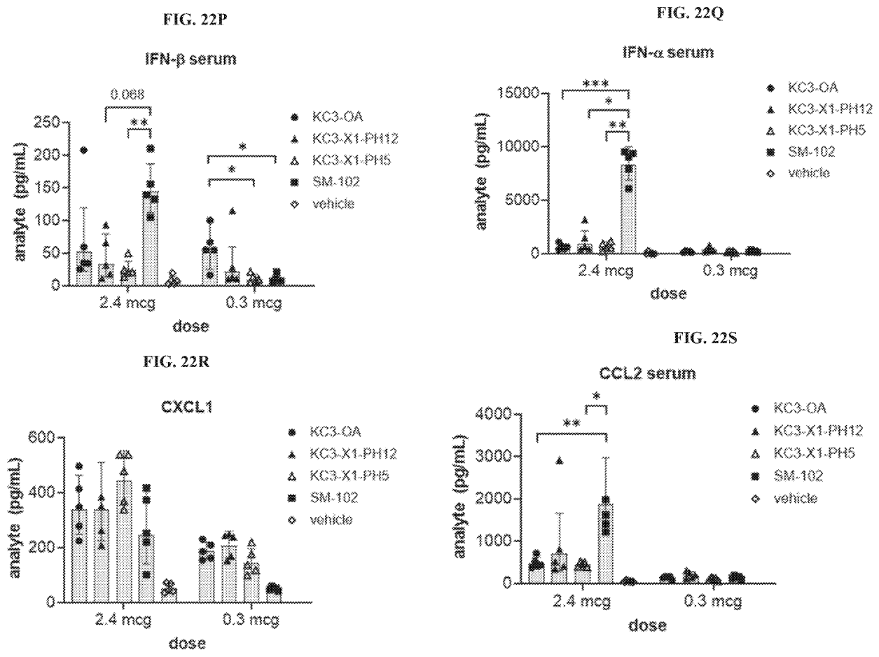

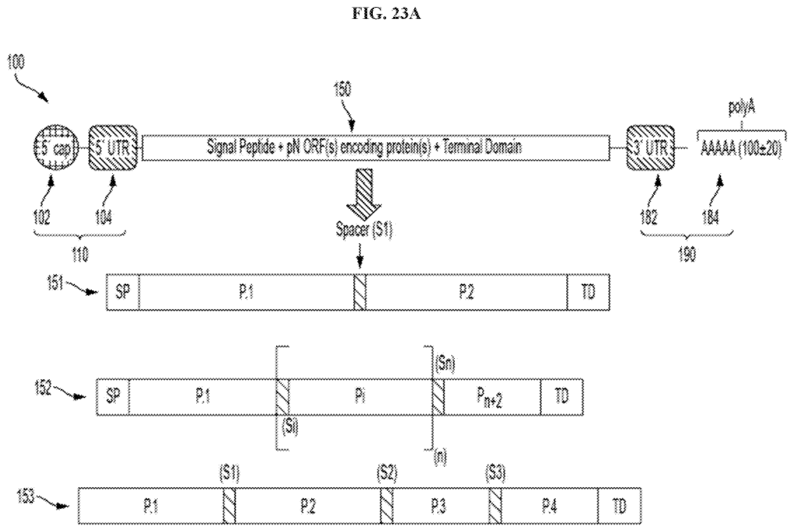

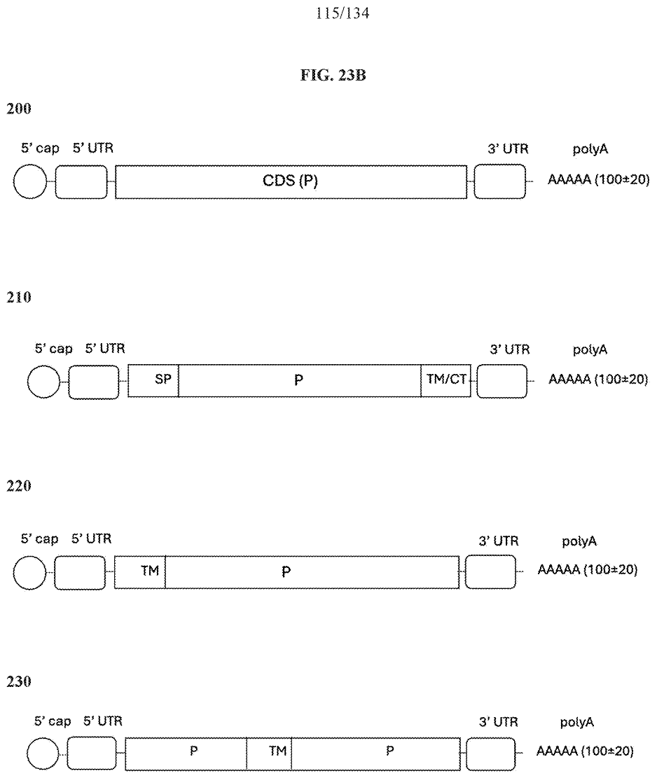

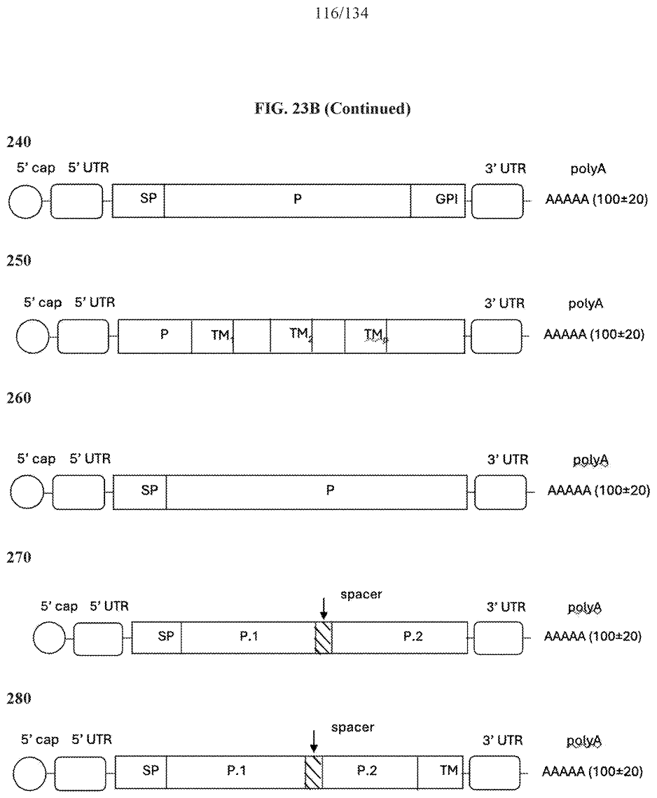

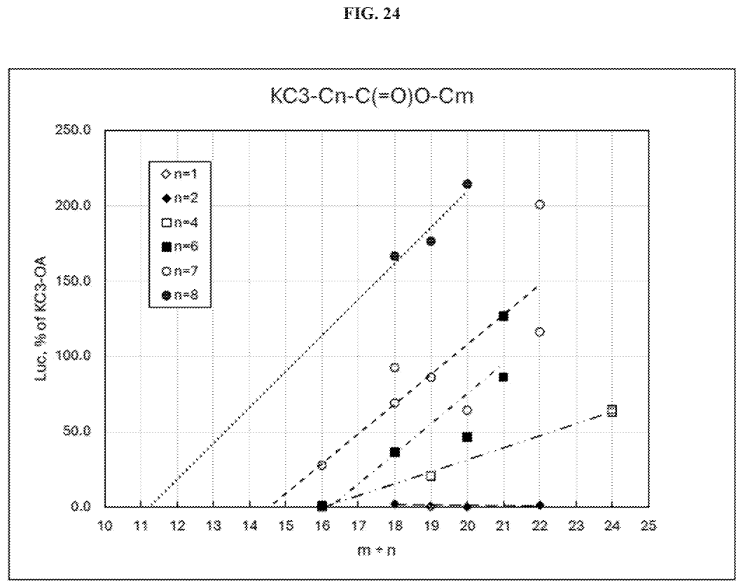

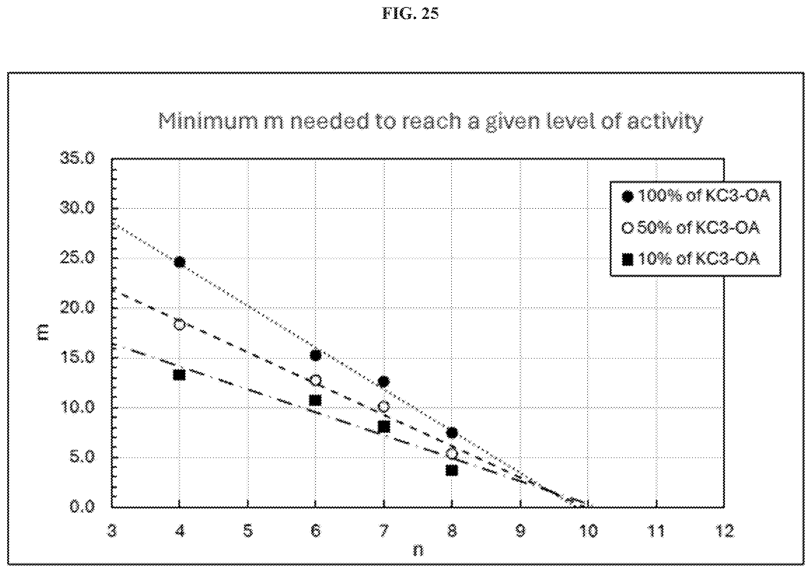

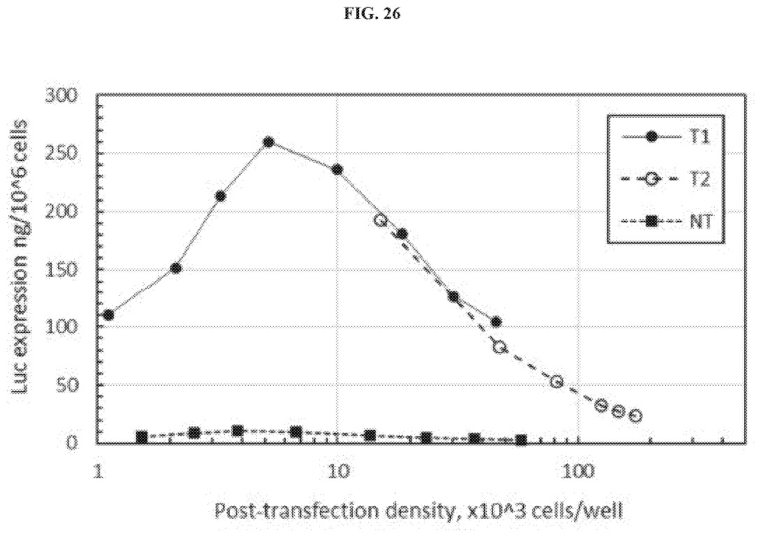

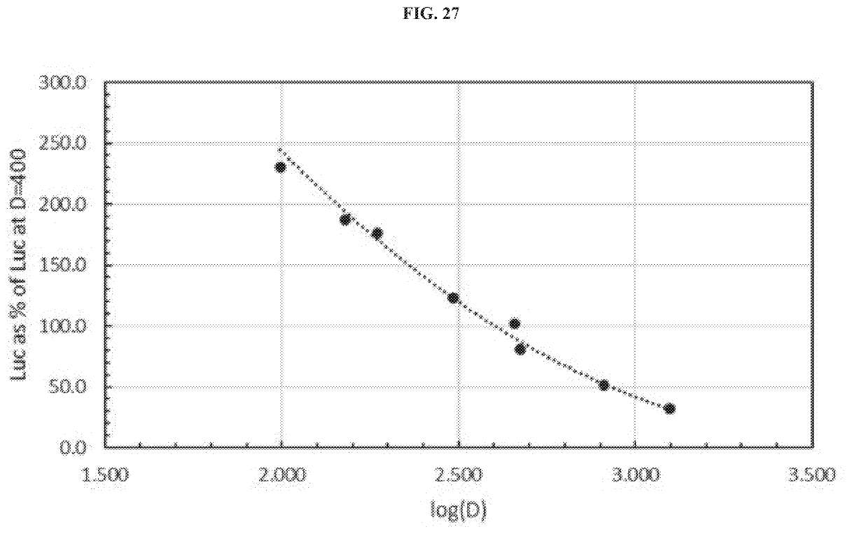

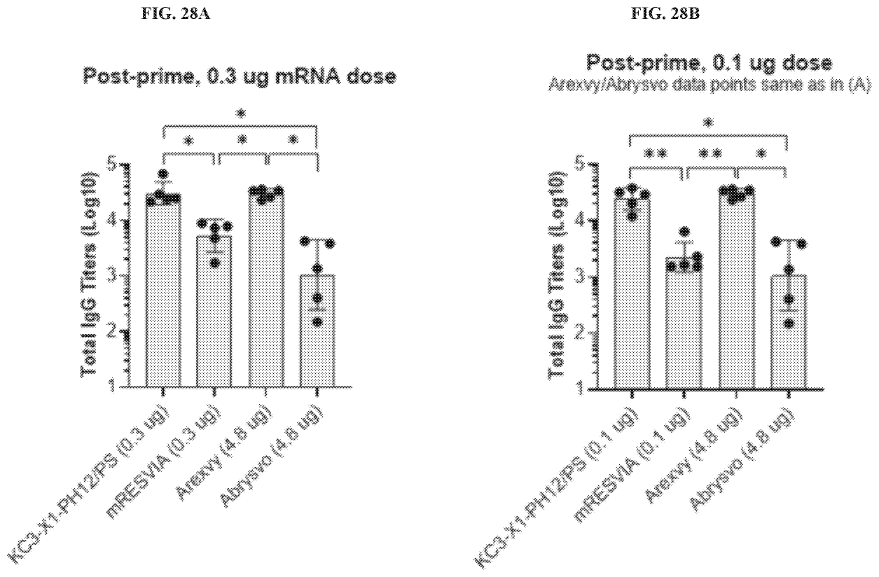

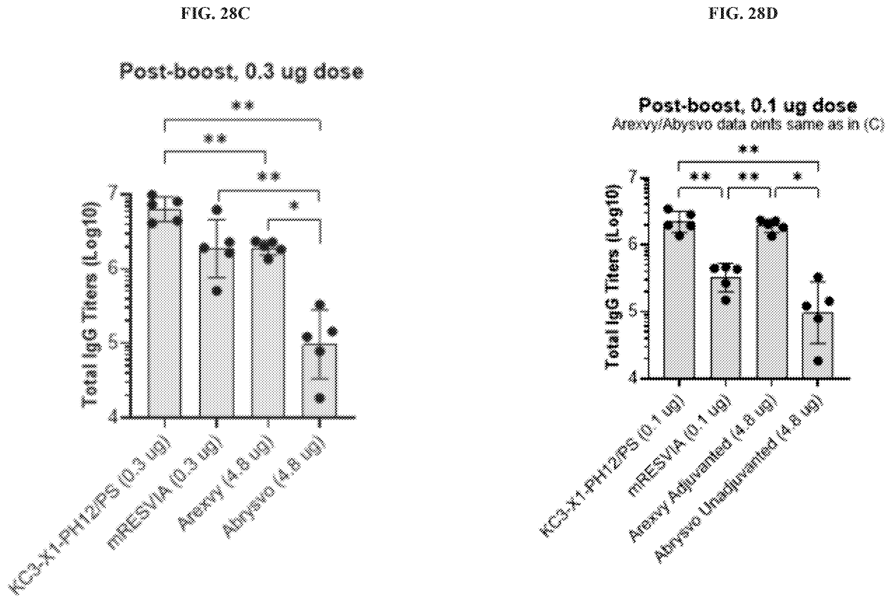

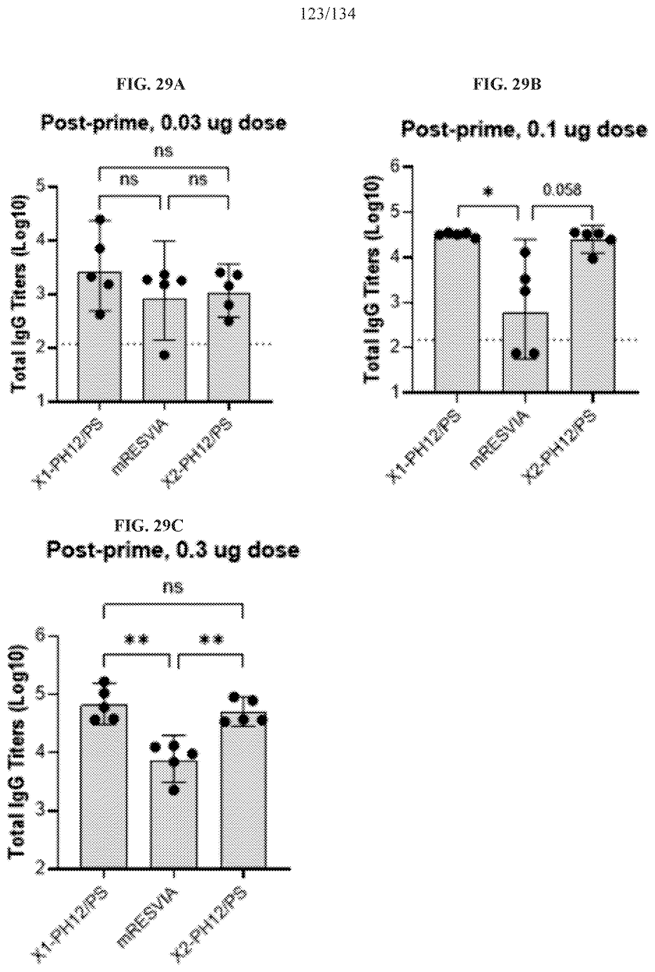

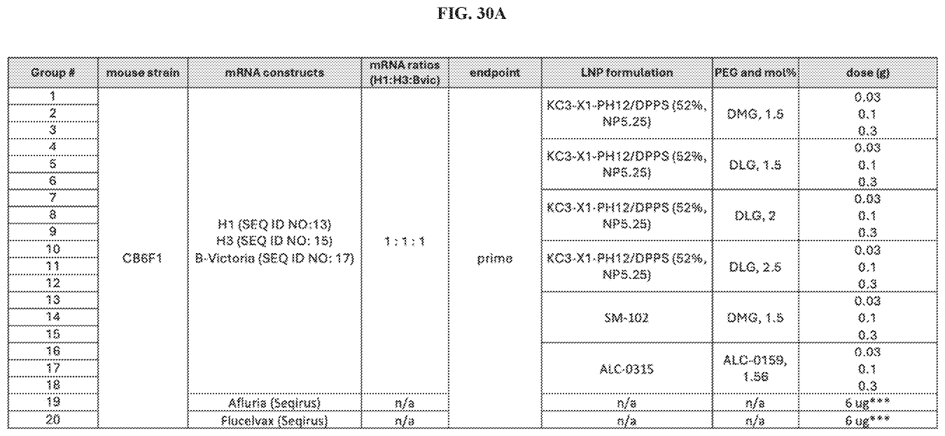

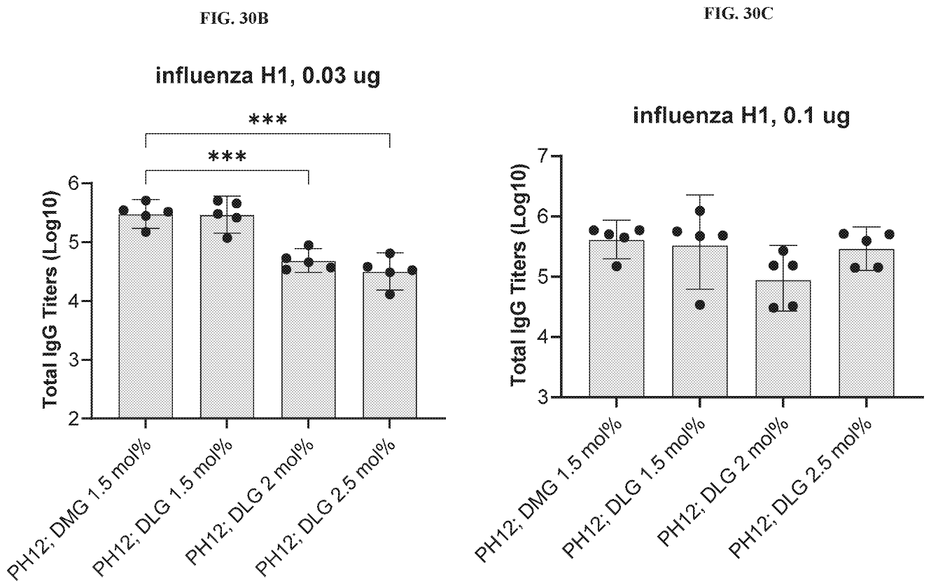

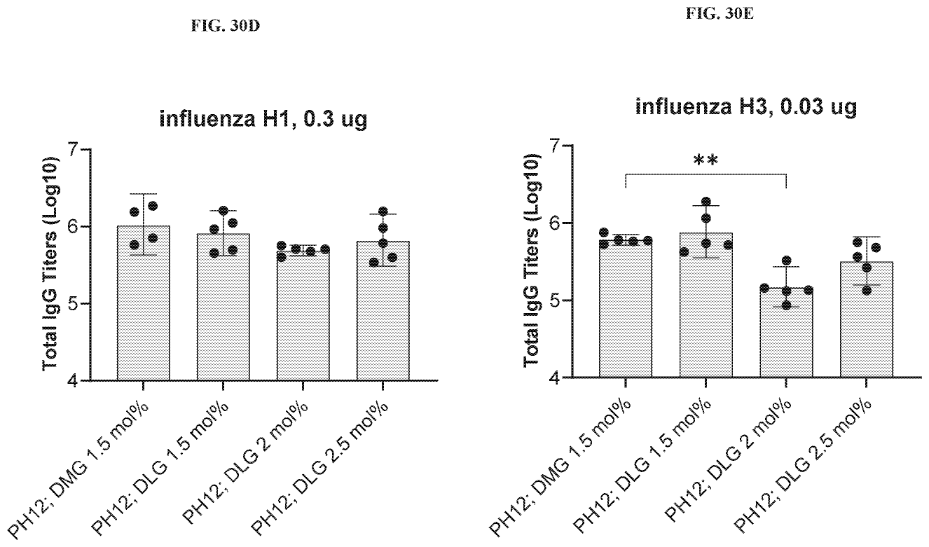

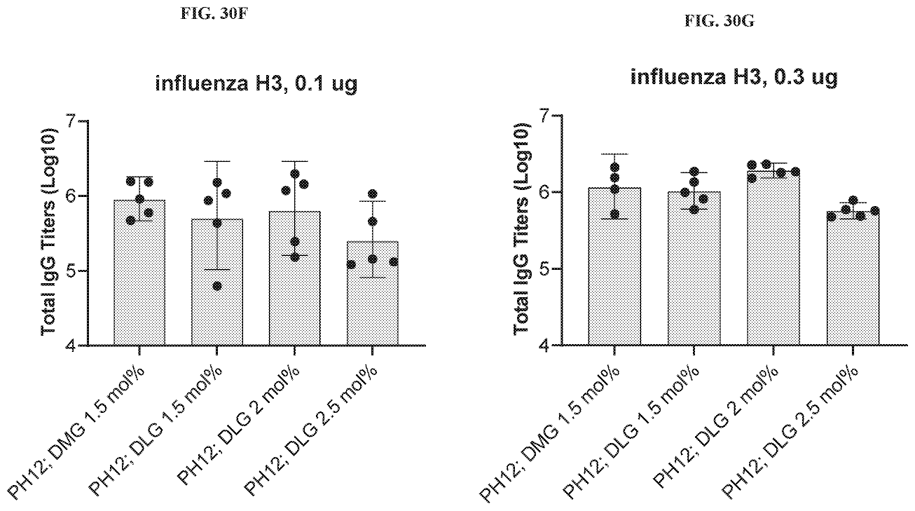

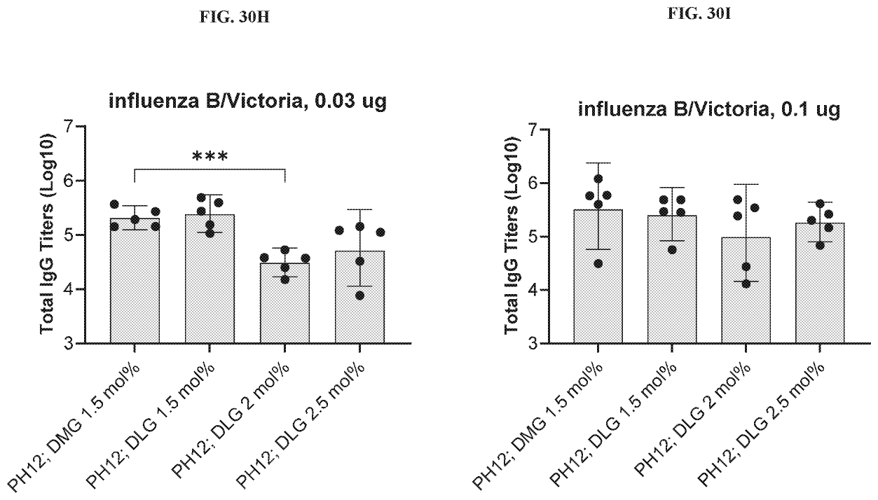

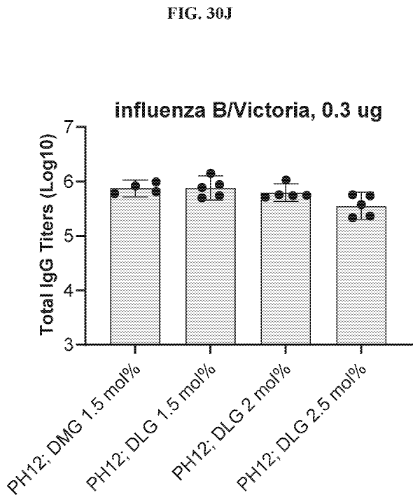

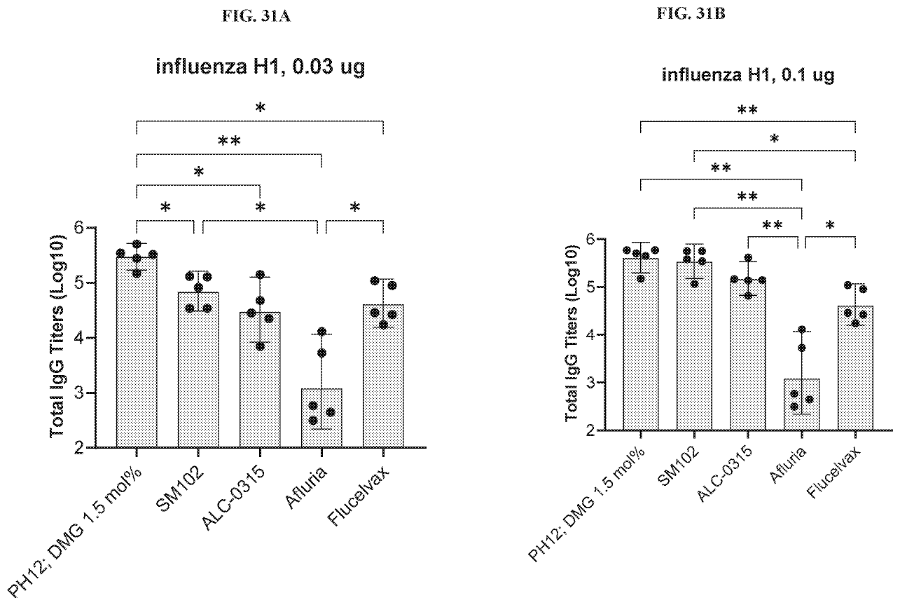

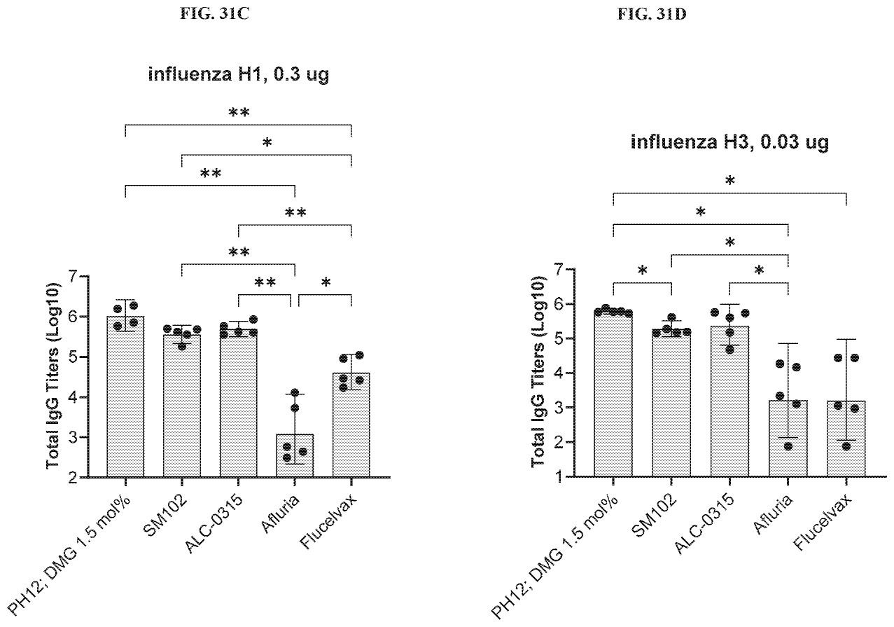

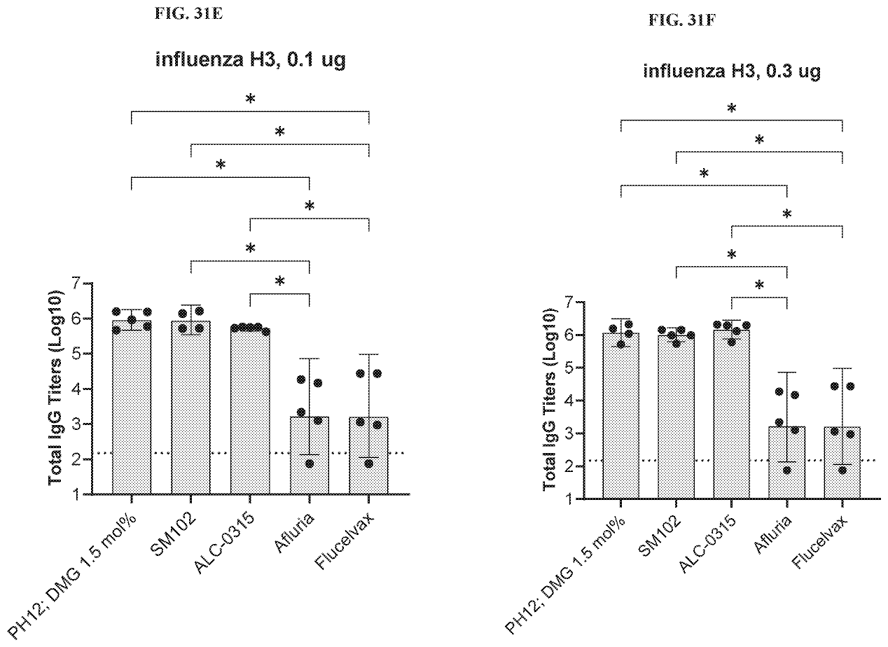

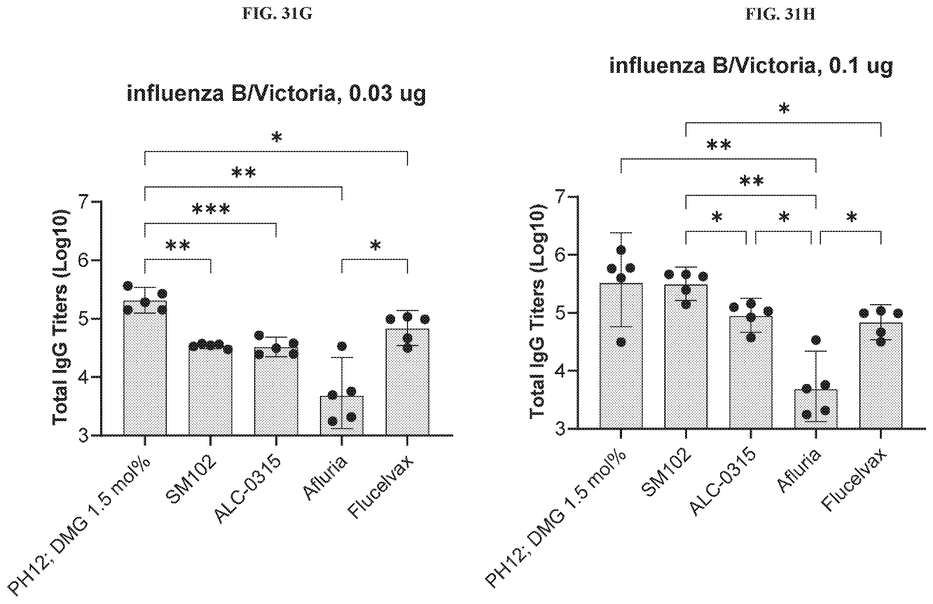

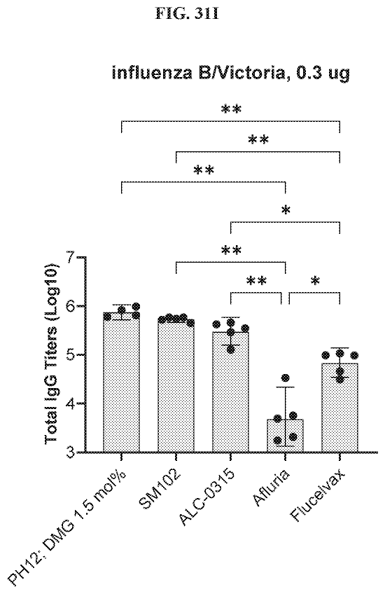

In addition to humoral immunity, evaluating CD4⁺ and CD8⁺ T cell responses is essential for understanding the full immunological impact of LNP-encapsulated mRNA vaccines encoding an H5 antigen. CD4⁺ T helper cells support the development of high-affinity, class-switched IgG antibodies and durable immune memory through their role in germinal center formation and B cell maturation. Concurrently, CD8⁺ cytotoxic T lymphocytes contribute to cellular immunity by recognizing and eliminating antigen-expressing cells, which is particularly important for protection against intracellular pathogens such as influenza viruses. Robust CD4⁺ and CD8⁺ responses indicate efficient antigen processing and presentation via both MHC class II and class I pathways, reflecting effective mRNA translation and innate immune activation by the LNP formulation. Together, these T cell responses complement antibody measurements by providing insight into the breadth, quality, and durability of vaccine-induced immunity. The H5-specific T cell responses were also analyzed in the spleen. Interestingly, the magnitude of the CD4 T cell responses in the 0.1 μg groups positively correlated with the mol % of KC3-X1-PH5, suggesting that 48 mol % may be better suited for B cell responses and 56 mol % may be better for T cells (FIG. 17F). There was a positive trend in the CD8 response as the mol % of KC3-X1-PH5 increased, but this was not statistically significant (FIG. 17H). In the 0.02 μg dose groups, the CD4 (FIG. 17G) and CD8 (FIG. 171) responses were smaller than at the 0.1 μg dose level, but there was no difference in the magnitude between the formulations. In conclusion, all tested biodegradable ICLs could be formulated in mRNA-LNPs that were immunogenic in vivo. The mol % of KC3-X1-PH5 appeared to be influence the antibody and T cell responses: a lower mol % may be better for eliciting vaccine antibodies, whereas a higher mol % may be better for T cell responses.

In Example 11E, mice were dosed intramuscularly with either 0.1 or 0.02 μg (see FIG. 18 for study design). Serum was collected on study day (SD) 21 for measuring antibody (Ab) titers and mice were then given a homologous boost immunization. Serum was collected 2 weeks post boost (SD35) and spleens were collected on SD49 to measure H5-specific T cell responses. While only statistically significant compared to some groups, the SM-102 formulation tended to have the lowest binding IgG titers, both at the 0.1 and 0.02 μg doses (FIGS. 19A-19D). At both doses PH12 was generally more immunogenic when used at 52 or 56 mol % and there was not a consistent difference in Ab titers when an N/P ratio of 4.5 or 5.25. For PH5, at the 0.1 μg dose this ICL was immunogenic at 48, 52 and 56 mol %. At the 0.02 μg dose, 48 and 52 mol % with an N/P of 5.25 seemed to be preferable. T cell responses in the spleen were only quantified in mice vaccinated with PH12 and PH5 LNPs using 52 or 56 mol %, and these were compared to the SM-102 formulation. Overall, all formulations elicited similar magnitude H5-specific CD4 and CD8 T cells (FIGS. 19E and 19F). These data indicate that B cell responses and circulating Ab levels may be more sensitive to small changes in immunogenicity caused by variations in the ICL mol % and N/P ratio. In contrast, at least at the doses tested, T cell expansion may be less sensitive. It was determined that lyophilized and reconstituted samples retained ~90% activity of the frozen samples, while the LNP size increased ~30 nm after reconstitution and the % EE was reduced by approximately 6% compared to the frozen or refrigerated material. Multiple freeze-thaws of the frozen LNPs did not impact activity. Non-frozen LNPs after 7 days at 2-8° C. were as active as the frozen reference, if not better. There were no clear trends observable from mixing the LNPs at either concentration, or whether filtering the lipid mixture ethanolic solution made a difference.

In Example 11F (FIGS. 22A-22J), Intramuscular injection of all four LNP formulations increased local and systemic concentrations of canonical pro-inflammatory cytokines and chemokines, and 2.4 mcg mRNA-LNPs induced higher levels than the 0.3 mcg dose. In the dLNs, there was upregulation of IFN-g (FIG. 22B), TNF-α (FIG. 22C), IL-6 (FIG. 22E), IL-1b (FIG. 22F), IFN-b (FIG. 22G), IFN-α (FIG. 2211), and the chemokines CXCL1 (FIG. 221) and CCL2 (FIG. 22J). Analytes concentrations between formulations were statistically similar with the exception of type I IFN-α and IFN-b: SM-102 induced significantly higher concentrations than all three of the KC3-based ICLs. Systemically (blood serum concentrations), the same analytes were upregulated (see FIGS. 22K-22S). SM-102 induced statistically significantly higher concentrations of IFN-g (FIG. 22K), TNF-α (FIG. 22L), IFN-b (FIG. 22P), IFN-α (FIG. 22Q), and CCL2 (FIG. 22S) than the KC3-based ICLs. There was no statistical difference between KC3-OA, KC3-X1-PH5 and KC3-X1-PH12. Altogether, these data show that SM-102 and the KC3-based ICLs signal through the same pathways, but that SM-102 is more inflammatory, both locally and systemically. High blood levels of these cytokines could lead to increased chances of reactogenicity such as fever and chills after vaccination with SM-102 compared to KC3-OA, KC3-X1-PH5 and KC3-X1-PH12.

In a second study (Example 12), a multivalent mRNA vaccine formulation combined four antigens—two influenza A H1 constructs, an H3 antigen, a B/Victoria-like hemagglutinin, and a stabilized RSV preF protein—to evaluate broader immune coverage and the ability of LNP systems to support simultaneous expression of multiple immunogens. Immune responses were assessed through antibody titers and cytokine profiles, allowing comparison of how formulation differences impact both the breadth and balance of immunity across multiple viral targets.

In Example 12, mRNAs encoding antigens for influenza A subtypes H1 (SEQ ID NO:13 encoding SEQ ID NO:12), H3 (SEQ ID NO:15 encoding SEQ ID NO:14), influenza B/Victoria (SEQ ID NO:17 encoding SEQ ID NO:16), and RSVpreF (SEQ ID NO:10 encoding SEQ ID NO:9) were combined at equal weight ratios and encapsulated into LNPs comprising ionizable lipids of Formula (I). To measure the ability of these mRNA-LNPs to induce antibodies, BALB/c mice were immunized at a high and 3-fold lower dose in the caudal thigh in a volume of 50 L. The experimental design and dosing schema is shown in FIG. 20A. Twenty-one days post vaccination, serum was collected and stored at −80° C. until analysis. Mouse IgG endpoint antibody titers against the matching H1, H3, B-Victora, RSVpreF or H5 antigens were determined by standard ELISA. Results are shown in FIGS. 20B-20H and discussed in Example 12A.

In another study (Example 14), a trivalent influenza-focused formulation encoding H1, H3, and B/Victoria antigens was evaluated to more directly model seasonal influenza vaccination. Measurements included endpoint antibody titers at multiple timepoints and cellular responses, providing a comprehensive view of how LNP composition affects immunogenicity against clinically relevant influenza strains. Collectively, these studies demonstrate the utility of mRNA-LNP platforms for mono-, multi-, and combination antigen vaccines, while highlighting the role of ionizable lipid selection in modulating immune potency, breadth, and durability.

Example 14A was performed (FIG. 30A) primarily to determine whether PEG-DLG could be used in place of PEG-DMG. While PEG-DMG is the most common form used in LNPs, there may be advantages to using PEG-DLG instead: since PEG-DLG has a shorter lipid tail (C12), it should dissociate faster from the LNP after injection. This could decrease the ability of PEG to cross-link B cell receptors and decrease the chance of developing anti-PEG Abs and potentially reduce vaccine reactogenicity. The immunogenicity of these test articles were benchmarked against the ALC-0315 and SM-102 comparator formulations and against standard trivalent inactivated split-virus influenza vaccines. FIGS. 30B-30D show Ab titers for H1, FIGS. 30E-30G show Ab titers for H3, and FIGS. 30H-30J show Ab titers for B/Victoria. For all HA antigens, IgG titers were comparable between the KC3-X1-PH12/PS compositions with 1.5 mol % of either PEG-DMG or PEG-DLG. For all HA subtypes at the lowest 0.03 μg dose, LNPs comprised of 2.0 mol % PEG-DLG had significantly lower titers, indicating that this mol % decreased the immunogenicity of the mRNA-LNP formulation; this was only evident at low doses when Ag levels were limiting. Titers for the 2.5 mol % PEG-DLG were only significantly reduced relative to 1.5 mol % PEG-DMG for H1 at 0.03 μg. In conclusion, the KC3-X1-PH12/PS LNP formulation with PEG-DLG at 1.5 mol % is just as immunogenic as PEG-DMG at 1.5 mol %. PH12/PS LNPs comprised with PEG-DLG also had similar physicochemical properties to those of PEG-DMG LNPs, indicating they formed “well-behaved” LNPs and there was no obvious deleterious effect of switching from DMG to DLG.

Example 14B was performed primarily to compare the immunogenicity of LNPs formulated with KC3-X1-PH12/PS PEG-DMG (1.5 mol %) with the immunogenicity of comparator LNP formulations using the SM-102 or ALC-0315 ICLs and to benchmark their immunogenicity to standard inactivated split-virus trivalent seasonal influenza vaccine. We used trivalent egg-based (Afluria™) or cell-based (Flucelvax™) vaccines manufactured by Seqirus. These vaccines and the mRNA test articles use the same H1 and B/Victoria HA antigens, but it is important to note these Seqirus vaccines use updated influenza A/H3N2 strains than what is encoded by mRNA that differ by 12-13 amino acids. FIGS. 31A-31C show total anti-H1 Ab titers, FIGS. 31D-31F show total anti-H3 Ab titers, and FIGS. 31G-31I shows total anti-B/Victoria Ab titers. Note that the data points for Afluria and Flucelvax are duplicated between all plots for each HA subtype—this was done for statistical comparison purposes. The KC3-X1-PH12/PS PEG-DMG mRNA-LNP generally elicited the highest IgG binding Abs at the 0.03 μg dose compared to all other formats. As the dose increased, Ab titers between the mRNA vaccines became similar, which is what we have observed for other antigens—as Ag levels increase in the draining LNs, differences in LNP delivery/transfection efficiency are no longer a limiting factor and Ab titers converge to a similar level. In almost all cases Ab titers elicited by the KC3-X1-PH12/PS formulation induced higher levels than either Afluria or Flucelvax. In conclusion, the KC3-X1-PH12/PS LNP platform can be used to encapsulate multiple mRNAs encoding vaccine antigens. This formulation generates B cell responses more efficiently than the comparator SM-102 and ALC-0315 mRNA-LNP formulations, and this is most evident at low dose levels when antigen expression becomes a limiting factor. It is also far more efficient than traditional multivalent split-virus vaccines. This is particularly important in pandemic situations where people do not have pre-existing immunity.

### RSV Studies

RSV is the leading cause of severe respiratory infection in children under 5 years of age and is a major cause of respiratory disease and death in the elderly. Most neutralizing Abs in recovered individuals target epitopes that are only exposed in the prefusion conformation of the fusion (F) glycoprotein (herein called preF). Since F is metastable and moves between pre- and post-fusion conformations, it has been shown that targeted mutations locking F in a prefusion conformation greatly improve B cell responses to neutralizing epitopes. Protein subunit and mRNA vaccines using the preF antigen are efficacious in preventing severe respiratory disease in infants (maternal immunization) and in the elderly.

In the multivalent influenza/RSV study (Example 12), the RSV preF antigen was included alongside influenza H1, H3, and B/Victoria hemagglutinin mRNAs to evaluate immune responses in a combined formulation. Immune readouts focused on total IgG titers against RSV preF, allowing assessment of humoral immunity elicited by the LNP-encapsulated mRNA within a multivalent context. The results demonstrated that the inclusion of RSV preF mRNA in the quadrivalent formulation elicited robust antigen-specific antibody responses comparable to those observed for the influenza antigens, indicating that the LNP platform can effectively deliver multiple mRNA antigens simultaneously without compromising immunogenicity. These findings support the potential of mRNA-LNP vaccines for combination approaches targeting either/both influenza and/or RSV.

In Example 13, an immunogenicity study was conducted to compare the performance of the KC3-X1-PH12/PS mRNA-LNP formulation encoding RSV preF with that of commercially approved RSV vaccines. The comparators included mRESVIA™ (Moderna, SM-102 LNP mRNA vaccine), Arexvy™ (GSK, adjuvanted protein subunit vaccine with AS01E), and Abrysvo™ (Pfizer, non-adjuvanted protein subunit vaccine). The primary readout was total IgG titers, which reflect the magnitude of the humoral immune response elicited by each formulation. This design allowed direct comparison of the mRNA-LNP approach with established protein-based vaccines in terms of inducing robust antibody responses, providing insight into the relative immunogenicity of KC3-X1-PH12/PS LNPs for RSV preF antigen delivery.

Example 13A is a RSVpreF immunogenicity study conducted primarily to compare the KC3-X1-PH12/PS mRNA-LNP formulation with commercially approved preF vaccines: mRESVIA™ (Moderna, SM-102 LNP formulation), Arexvy™ (GSK, protein subunit, adjuvanted with AS01E) and Abrysvo™ (Pfizer, protein subunit non-adjuvanted). BALB/c mice were dosed i.m. with either 0.3 or 0.1 μg of KC3-X1-PH12/PS mRNA-LNPs or mRESVIA. The physicochemical properties the KC3-X1-PH12/PS mRNA-LNPs are shown in Table 13A-1. Mice were also dosed i.m. with 4.8 μg Arexvy (preF protein, subtype A; contained 1 ag AS01E) or 4.8 μg Abrysvo (2.4 μg subtype A preF, 2.4 μg subtype B preF). Total binding IgG endpoint titers 4 weeks post-prime are shown in FIGS. 28A-28B. Mice were then boosted and endpoint titers 2 weeks post-boost are shown in FIGS. 28C-28D. Note that the data points for Arexvy and Abrysvo are duplicated between A/B and C/D for comparison purposes and statistical analysis. Vaccination with KC3-X1-PH12/PS elicited comparable or superior Ab titers to those of Arexvy, and both of these test articles were more immunogenic than mRESVIA (with the exception of post-boost titers at the 0.3 μg dose) and Abrysvo. Abrysvo and mRESVIA elicited the lowest Ab titers, and mRESVIA was only higher than Abrysvo post-boost at the 0.3 μg dose. In conclusion, the KC3-X1-PH12 PS-targeted LNP formulation is able to efficiently transfect antigen-presenting cells with mRNA encoding membrane RSVpreF antigen that results in potent B cell responses.

Example 13B is a RSVpreF immunogenicity study conducted primarily to compare PS-targeted mRNA-LNPs comprised of the ICL KC3-X1-PH12 or the ICL KC3-X2-PH12, which has the ester linkage in the lipid tail in the reverse orientation relative to KC3-X1-PH12. Clinical-grade mRESVIA was used as a benchmark comparator. No mice in the 0.01 μg dose group had titers greater than a 150-fold dilution, which was the lower limit of detection in this assay (data not shown) (FIGS. 29A, 29B and 29C). At the 0.03 μg dose level, all but one mouse in the mRESVIA group had detectable titers, and titers were similar between all three groups. The KC3-X1-PH12 and —X₂—PH12 formulations were more immunogenic than mRESVIA at 0.1 and 0.3 ug. In conclusion, LNPs comprised of KC3-X1-PH12 and KC3-X2-PH12 were similarly immunogenic, and both were superior to mRESVIA, which uses the SM-102 ICL. Interestingly, the superior immunogenicity of the PH12 LNPs over mRESVIA was lost at 0.03 μg and lower. One potential explanation is the PH12 LNPs were nearing their lower immunogenicity thresholds and thus were not able to outperform mRESVIA.

### Dengue Virus Studies

Development of a dengue virus (DENV) mRNA-LNP vaccine (Example 15) focuses on addressing the unique immunological challenges posed by the four distinct DENV serotypes. Because initial infection is often mild but secondary infection with a different serotype can cause severe disease—largely due to antibody-dependent enhancement from cross-reactive but poorly neutralizing antibodies—a safe and effective vaccine must elicit balanced neutralizing antibodies (nAbs) across all four serotypes. In addition to humoral immunity, T cells can provide complementary protection by recognizing conserved non-structural (NS) proteins that are not accessible to antibodies, supporting cross-serotype cellular immunity. mRNA vaccines are particularly well-suited for this purpose, as they are capable of inducing strong T cell responses. These responses can be further enhanced by engineering the mRNA-encoded antigens to localize to endosomal and lysosomal compartments, improving peptide presentation and T cell activation. Collectively, this strategy leverages both broad humoral and cellular immunity to maximize safety and efficacy against all DENV serotypes.

### Malaria Studies

Development of a malaria mRNA-LNP vaccine focuses on eliciting immunity against multiple stages of the Plasmodium life cycle to maximize protective efficacy. Following a mosquito bite, sporozoites infect hepatocytes, initiating the liver stage, which then progresses to the erythrocytic stage as merozoites replicate in red blood cells. Protein antigens expressed at each stage can be targeted by the adaptive immune system. Current liver-stage vaccines, such as RTS,S and R21, induce B- and T-cell responses against the circumsporozoite protein (CSP) to block infection of hepatocytes. Blood-stage vaccine strategies aim to generate neutralizing antibodies against merozoite antigens like PfRH5 and PfMSP10 to prevent red blood cell invasion. A multivalent mRNA-LNP vaccine can encode antigens from both liver and blood stages, leveraging the platform's ability to elicit robust humoral and cellular immunity simultaneously, thereby providing a broader and potentially more effective protective response against malaria.

### 6. Preparing LNP Compositions

The method of making an LNP can comprise mixing a first solution with a second solution. Mixing can be achieved using standard liquid mixing techniques, such as propellor mixing, vortexing solutions or preferably through microfluidic mixing or high efficiency T-mixing. In some embodiments, the first solution comprises a lipid or a plurality of lipids and a nucleic acid, where all components are solubilized, in water/solvent system. The solvent may be any water miscible solvent (e.g., ethanol, methanol, isopropanol, acetonitrile, dimethylformamide, dimethylsulfoxide, dioxane or tetrahydrofuran). In some embodiments, the first solution comprises a small percentage of water or pH buffered water. In some embodiments, the first solution comprises a single type of lipid, for example, an ionizable lipid, a phospholipid, a sterol, or a PEG-containing lipid. In some embodiments, the first solution comprises a plurality of lipids. In some embodiments, the plurality comprises an ionizable lipid, a phospholipid, a sterol, or a PEG-containing lipid. In some embodiments, the second solution is water. In some embodiments, the second solution is an aqueous buffer with a pH between 3-6 (e.g., a pH of about 3, about 4, about 5, or about 6). The second solution may comprise a load component, e.g., a nucleic acid (e.g., mRNA). The second solution may comprise a small percentage of water-miscible organic solvent. In some embodiments, the solution comprising a mixture of the first and second solutions comprising the LNP suspension can be diluted. In some embodiments, the pH of the solution comprising a mixture of the first and second solutions comprising the LNP suspension can be adjusted. Dilution or adjustment of the pH of the LNP suspension can be achieved with the addition of water, acid, base or aqueous buffer. In some embodiments, no dilution or adjustment of the pH of the LNP suspension is carried out. In some embodiments, both dilution and adjustment of the pH of the LNP suspension is carried out. In some embodiments, excess reagents, solvents, unencapsulated nucleic acid maybe removed from the LNP suspension by tangential flow filtration (TFF) (e.g., diafiltration). The organic solvent (e.g., ethanol) and buffer may also be removed from the LNP suspension with TFF. In some embodiments, the LNP suspension is subjected to dialysis and not TFF. In some embodiments, the LNP suspension is subjected to TFF and not dialysis. In some embodiments, the LNP suspension is subjected to both dialysis and TFF. In one aspect, the present disclosure features a method comprising treating a sample of LNPs comprising nucleic acid, with a fluid comprising a detergent (e.g., Triton X-100, or anionic detergents (such as, but not limited to, sodium dodecyl sulfate (SDS), or non-ionic detergent, such as but not limited to β-octylglucoside, or Zwittergent 3-14) for a period of time suitable to degrade the lipid layer and thereby release the encapsulated and/or entrapped nucleic acid(s). In an embodiment, the method further comprises analyzing the sample for the presence, absence, and/or amount of the released nucleic acid(s).

Methods of making lipid nanoparticle (LNP) compositions encapsulating mRNA are provided in Example 7 (Preparing ligand-targeted LNPs) and Example 9 (Preparation of lipidic nanoparticles) of Patent Cooperation Treaty Patent Application PCT/US2021/060877 (WO2022/115645), are incorporated herein by reference in their entirety. Methods of making lipid nanoparticle (LNP) compositions encapsulating mRNA are provided in Example 7 (Preparing ligand-targeted LNPs) and Example 9 (Preparation of lipidic nanoparticles) of Patent Cooperation Treaty Patent Application PCT/US2023/067517 (WO2023/230587), are incorporated herein by reference in their entirety. Methods of characterizing lipid nanoparticle (LNP) compositions encapsulating mRNA are provided in Example 10 (LNP Characterization) and Example 11 (Determination of transfection efficiency in murine dendritic cells of LNPs using mCherry mRNA) of Patent Cooperation Treaty Patent Application PCT/US2021/060877 (WO2022/115645), are incorporated herein by reference in their entirety. Methods of making lipid nanoparticle (LNP) compositions encapsulating mRNA are provided in Example 10 (LNP Characterization) and Example 11 (Determination of transfection efficiency in murine dendritic cells of LNPs using mCherry mRNA) of Patent Cooperation Treaty Patent Application PCT/US2023/067517 (WO2023/230587), are incorporated herein by reference in their entirety. These methods can be modified by using a microfluidics chip from ChipShop (187) to make LNPs at flow rates of 18 mL/min instead of the Nanoprecision Systems microfluidics cassette at 6 ml/min whilst keeping downstream processing and analytical procedures the same as described in the published patents referenced above. Additional disclosure of methods for the manufacture of mRNA lipid nanoparticle compositions are provided in: S. J. Shepherd, X. Han, A. J. Mukalel, R. El-Mayta, A. S. Thatte, J. Wu, M. S. Padilla, M. Alameh, N. Srikumar, D. Lee, D. Weissman, D. Issadore, & M. J. Mitchell, Throughput-scalable manufacturing of SARS-CoV-2 mRNA lipid nanoparticle vaccines, Proc. Natl. Acad. Sci. U.S.A. 120 (33) e2303567120, https://doi.org/10.1073/pnas.2303567120 (2023), incorporated by reference herein in its entirety.

### 7. Pharmaceutical Compositions

In some embodiments, the LNP can be formulated as an injectable pharmaceutical composition, the LNP composition as the active pharmaceutical ingredient (API) with a set of excipients that ensure stability, safety, and suitability for administration. These typically include buffering agents such as phosphate, acetate, or TRIS to maintain physiological pH during storage and injection. Tonicity-adjusting agents, most commonly sucrose, trehalose, or sodium chloride, are added to ensure the formulation is isotonic and to help protect the LNPs during freezing or thawing. Cryoprotectants or stabilizing sugars may also be used to preserve particle integrity throughout manufacturing, shipping, and storage. The final formulation is prepared in sterile water for injection or another pharmaceutically acceptable aqueous carrier and may include trace chelating agents or antioxidants if needed for long-term stability. Together, these components create a biocompatible, sterile, and stable injectable solution in which the LNP API can be safely and effectively delivered.

A lipid nanoparticle (LNP) vaccine encapsulating mRNA encoding an antigen is a pharmaceutical composition designed to deliver the mRNA safely and efficiently to cells, where it can be translated into the target antigen to elicit an immune response. The mRNA encapsulated in the LNP can encodes a protein antigen specific to the pathogen of interest. The LNP formulation typically includes (a) ionizable cationic lipids in an amount effective to enable encapsulation of the negatively charged mRNA and promote endosomal escape after cellular uptake; (b) one or more phospholipids in an amount effective to form the structural bilayer of the nanoparticles; (c) cholesterol in an amount effective to provide stability and rigidity to the particle membrane; and a PEG-lipid in an amount effective to improve particle dispersion and reduce aggregation, enhancing stability and circulation. The vaccine may also include buffers, such as phosphate-buffered saline or citrate, to maintain pH, and salts such as sodium chloride to maintain isotonicity. Sugars or polyols, such as sucrose or trehalose, can be included as stabilizers and cryoprotectants to preserve LNP integrity during storage and handling.

In some embodiments, LNPs are administered into a subject at a dose of about 0.01 to about 5 mg/kg mRNA by any route of administration known in the art and/or outlined above. According to some embodiments, a proportion of LNPs are taken up dendritic cells (DC), while some may accumulate in the liver and spleen. The DC cells can express the antigenic peptide, process it for MHC I presentation and travel to the lymph node for presentation to naïve T cells inducing an education of memory T-cells towards the antigen.

In some embodiments, a pharmaceutical composition is a lipid nanoparticle (LNP) composition comprises a mRNA nucleic acid encapsulated at a N/P ratio of 5.25 in lipidic nanoparticles consisting of: 56 mol % of the KC3-X1-PH12 ionizable cationic lipid, 5 mol % DPPS, 5 mol % DSPC, 1.5 mol % PEG-DMG and 32.5 mol % cholesterol.

### 8. Methods of Treatment

A general method of treatment using an LNP vaccine encapsulating mRNA encoding an antigen involves administering a therapeutically effective amount of the vaccine to a subject in need of immunization or enhanced immunity against a target pathogen. Upon administration—typically via intramuscular or subcutaneous injection—the lipid nanoparticles facilitate delivery of the mRNA into host cells, where the mRNA is translated into the encoded antigen. Expression of the antigen by host cells triggers both humoral and cellular immune responses, including the production of antigen-specific antibodies and activation of CD4⁺ and CD8⁺ T cells. The method can be applied prophylactically to prevent infection or disease, or, in certain contexts, therapeutically to boost immunity in individuals with pre-existing exposure. Dosage, timing, and number of administrations are determined based on the specific antigen, formulation, and desired immune response, and may involve single or multiple doses to achieve optimal protection.

In some embodiments, a method of treatment comprises administering to a subject (human or animal) a therapeutically effective amount of an immunogenic lipid nanoparticle (LNP) composition comprising a mRNA nucleic acid encapsulated at a N/P ratio of 5.25 in lipidic nanoparticles consisting of: 56 mol % of the KC3-X1-PH12 ionizable cationic lipid, 5 mol % DPPS, 5 mol % DSPC, 1.5 mol % PEG-DMG and 32.5 mol % cholesterol.

Upon administration, typically via intramuscular injection, the LNPs facilitate delivery of the mRNA into host cells, where it is translated into the encoded antigens. Expression of these antigens induces robust cellular immune responses, including activation of antigen-specific CD4⁺ T cells that support macrophage activation and CD8⁺ T cells that contribute to killing infected cells, both of which are critical for controlling intracellular bacterial infection. The vaccine can be tailored to include multiple antigens to enhance breadth and durability of the immune response.

Expression of these antigens stimulates both humoral and cellular immune responses, including production of neutralizing antibodies and activation of antigen-specific CD4⁺ and CD8⁺ T cells, which together confer protection against infection and reduce disease severity. The vaccine can be formulated to include single or multiple influenza antigens to provide broad coverage across different strains and subtypes.

### Infuenza Vaccines

In some embodiments, a method of treating or preventing influenza comprising administering an LNP vaccine comprising a therapeutically effective amount of lipid nanoparticles encapsulating mRNA that encodes one or more influenza antigens, such as hemagglutinin (HA) from H1, H3, or B strains. Following administration, typically by intramuscular injection, the LNPs deliver the mRNA into host cells, where it is translated into the influenza antigens. Dosage and administration schedules can be adjusted to optimize immune responses, including single-dose or multi-dose regimens, to immunize against influenza.

A method of administering a seasonal influenza vaccine containing mRNA encoding multiple common influenza antigens involves delivering a therapeutically effective amount of lipid nanoparticles encapsulating the mRNAs to a subject, typically via intramuscular injection. The mRNA encodes hemagglutinin (HA) proteins from multiple circulating influenza strains, such as H1N1, H3N2, and B lineages, enabling broad immune coverage. Upon delivery, the LNPs facilitate uptake into host cells, where the mRNAs are translated into the respective antigens. Expression of these antigens stimulates both humoral and cellular immune responses, including production of strain-specific neutralizing antibodies and activation of CD4⁺ and CD8⁺ T cells. The vaccine can be administered as a single dose or in multiple doses to optimize the magnitude and durability of the immune response, providing protection against seasonal influenza infection and reducing disease severity.

### Mtb Vaccines

A method of treating or preventing Mycobacterium tuberculosis infection using an LNP vaccine involves administering a therapeutically effective amount of lipid nanoparticles encapsulating mRNA that encodes one or more tuberculosis antigens, such as Ag85A, Ag85B, ESAT-6, CFP-10, or synthetic multistage antigens designed to elicit broad immunity. Upon administration, typically via intramuscular injection, the LNPs facilitate delivery of the mRNA into host cells, where it is translated into the encoded antigens. Expression of these antigens induces robust cellular immune responses, including activation of antigen-specific CD4⁺ T cells that enhance macrophage activation and CD8⁺ T cells that contribute to killing infected cells, both of which are critical for controlling intracellular bacterial infection. The vaccine can be formulated to include multiple antigens to improve the breadth and durability of the immune response. Administration schedules, including single or multiple doses, can be optimized to maximize protective T cell-mediated immunity and reduce the risk of active tuberculosis disease.

### RSV Vaccines

A method of treating or preventing respiratory syncytial virus (RSV) infection using an LNP vaccine involves administering a therapeutically effective amount of lipid nanoparticles encapsulating mRNA that encodes one or more RSV antigens, such as the prefusion-stabilized F protein (preF) or the G glycoprotein. Upon administration, typically via intramuscular injection, the LNPs deliver the mRNA into host cells, where it is translated into the encoded antigens. Expression of these antigens stimulates both humoral and cellular immune responses, including the production of RSV-specific neutralizing antibodies and activation of antigen-specific CD4⁺ and CD8⁺ T cells, which together contribute to protection against infection and reduction of disease severity. The vaccine can be formulated to include single or multiple RSV antigens to enhance breadth and potency of the immune response. Administration schedules, including single or multiple doses, can be optimized to achieve rapid, durable, and protective immunity against RSV.

### Dengue Virus Vaccines

A method of treating or preventing dengue fever using an LNP vaccine involves administering a therapeutically effective amount of lipid nanoparticles encapsulating mRNA that encodes one or more dengue virus (DENV) antigens from the four serotypes (DENV-1, DENV-2, DENV-3, and DENV-4), such as the envelope (E) protein or non-structural (NS) proteins to elicit T cell responses. Upon administration, typically via intramuscular injection, the LNPs deliver the mRNA into host cells, where it is translated into the encoded antigens. Expression of these antigens induces balanced humoral immunity by generating neutralizing antibodies against all four DENV serotypes, while also stimulating cellular immune responses through activation of CD4⁺ and CD8⁺T cells that recognize conserved NS proteins, providing complementary cross-serotype protection. The vaccine can be formulated to include multiple antigens from all four serotypes to reduce the risk of antibody-dependent enhancement and to provide broad, durable immunity. Administration schedules, including single or multiple doses, can be tailored to optimize both antibody and T cell responses for effective prevention of dengue fever.

### Malaria Vaccines

A method of treating or preventing malaria using an LNP vaccine involves administering a therapeutically effective amount of lipid nanoparticles encapsulating mRNA that encodes one or more Plasmodium falciparum antigens from both liver and blood stages, such as the circumsporozoite protein (CSP) for the liver stage, and merozoite antigens like PfRH5 or PfMSP10 for the blood stage. Upon administration, typically via intramuscular injection, the LNPs deliver the mRNA into host cells, where it is translated into the encoded antigens. Expression of these antigens stimulates both humoral and cellular immune responses, including production of antigen-specific antibodies that can neutralize sporozoites or merozoites, and activation of CD4⁺ and CD8⁺ T cells that contribute to elimination of infected hepatocytes and red blood cells. The vaccine can be formulated to include multiple antigens from different stages of the parasite life cycle to enhance breadth and durability of the protective response. Administration schedules, including single or multiple doses, can be optimized to achieve robust, stage-spanning immunity capable of preventing infection and reducing malaria disease severity.

### Avian Flu Vaccines

A method of treating or preventing avian influenza using an LNP vaccine involves administering a therapeutically effective amount of lipid nanoparticles encapsulating mRNA that encodes one or more avian influenza virus antigens, such as hemagglutinin (HA) or neuraminidase (NA) proteins from relevant H5, H7, or H9 subtypes. Upon administration, typically via intramuscular injection, the LNPs deliver the mRNA into host cells, where it is translated into the encoded antigens. Expression of these antigens stimulates both humoral and cellular immune responses, including production of subtype-specific neutralizing antibodies and activation of antigen-specific CD4⁺ and CD8⁺ T cells, which together confer protection against infection and reduce disease severity. The vaccine can be formulated to include one or multiple antigens to provide broad coverage against different avian influenza strains. Administration schedules, including single or multiple doses, can be optimized to achieve rapid and durable immunity in subjects at risk of avian influenza infection.

Reference is made to PCT applications PCT/US2025/034395, filed Jun. 19, 2025, PCT/US2025/034670, filed Jun. 20, 2025, PCT/US2026/020233, filed Mar. 20, 2026, PCT/US2026/020236, filed Mar. 20, 2026, and to U.S. application Ser. No. 19/573,533, filed Mar. 20, 2026. All publications, patents and sequence database entries mentioned herein are hereby incorporated by reference in their entirety as if each individual publication or patent was specifically and individually indicated to be incorporated by reference.

It is to be understood that both the foregoing general description and the following detailed description are exemplary and explanatory only and are not restrictive of the compositions and methods of the present disclosure.

### ADDITIONAL EMBODIMENTS

In some embodiments, the ionizable cationic lipid is a compound of one or more of the following additional embodiments:
1. A lipid nanoparticle (LNP) composition comprising an ionizable lipid having a chemical structure

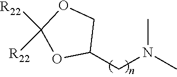

wherein n is 3 or 4, and
wherein each R₂₂ is independently a linear 15-22 member heteroalkyl or 15-22 member heteroalkylene comprising a bioreducible and/or hydrolysable moiety and optionally comprising one or two olefins.
2. The composition of embodiment 1, wherein R₂₂ is a linear C₁₅₋₁₉ heteroalkyl or heteroalkylene group comprising a bioreducible and/or hydrolysable moiety and optionally comprising one olefin or two olefins separated by at least two methylene groups.
3. The composition of embodiment 1, wherein each R₂₂ is independently a linear 15-19 member heteroalkyl or 15-19 member heteroalkylene comprising a bioreducible and/or hydrolysable moiety and optionally comprising one olefin.
4. The composition of any one of embodiments 1-3, wherein R₂₂ is a linear C₁₆ or C₁₈ heteroalkyl comprising a bioreducible and/or hydrolysable group, wherein the bioreducible and/or hydrolysable group comprises at least one carbon atom replaced with an oxygen, nitrogen, sulfur heteroatom or combination thereof.
5. The composition of embodiment 4, wherein R₂₂ is a linear C₁₈ heteroalkyl comprising a hydrolysable group, wherein the hydrolysable group comprises at least one carbon atom replaced with an oxygen heteroatom, and optionally further comprising a hydrolysable group and one olefin or two olefins separated by at least two methylene groups.
6. The composition any one of embodiments 1-5, wherein each R₂₂ is

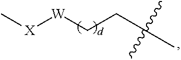

wherein d is 0-6,
wherein W is a bioreducible and/or hydrolysable group, and wherein X is a linear C₈₋₁₄ heteroalkyl or C₈₋₁₄ heteroalkylene group comprising a bioreducible and/or hydrolysable moiety and optionally comprising one or two olefins.
7. The composition embodiment 6, wherein each X is —(CH₂)ₐ— wherein (a+d) is 12 or 14.
8. The composition of embodiment 6, wherein each X is

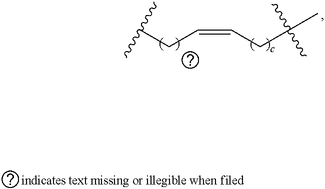

wherein (a+c+d) is 10 or 12.
9. The composition of any one of embodiments 1-8, wherein both R₂₂ groups are identical.
10. The composition of embodiment 9, wherein the bioreducible and/or hydrolysable group is —S—S—, —O—C(O)—, —C(O)—O— or a combination thereof.
11. The composition of embodiment 10, wherein the hydrolysable group is —O—C(O)—.
12. The composition of embodiment 10, wherein the hydrolysable group is —C(O)—O—.
13. The composition of embodiment 10, wherein the bioreducible group is —S—S—.
14. The composition of any one of embodiments 10-13, wherein n is 3.
15. The composition of any one of embodiments 10-13, wherein n is 4.
16. A lipid nanoparticle (LNP) composition comprising an ionizable lipid having a chemical structure

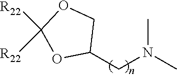

wherein:
   n is 3 or 4 and
   both R₂₂ are identical and are

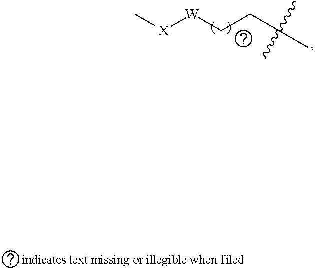

W is a bioreducible and/or hydrolysable group selected from the group consisting of —S—S—, —O—C(O)— and —C(O)—O—;
X is
   (A) —(CH₂)ₐ— wherein (a+c) is 12 or 14; or
   (B)

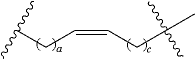

wherein (a+c+d) is 10 or 12.
17. The composition of embodiment 16, wherein n is 3.
18. The composition of embodiment 16, wherein n is 4.
19. A lipid nanoparticle (LNP) composition comprising an ionizable lipid having a chemical structure

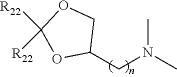

wherein n is 2, 3 or 4, and each R₂₂ is independently an 18-26 member heteroalkyl or heteroalkenyl chain comprising a bioreducible or hydrolysable group.
20. The composition of embodiment 19, wherein each bioreducible or hydrolysable group is an ester.
21. The composition of embodiment 19 or embodiment 20, wherein one or both R₂₂ comprise an olefin.
22. The composition of embodiment 21, wherein one or both R₂₂ comprise a single olefin.
23. The composition of embodiment 21, wherein one or both R₂₂ are heteroalkyl chains that do not comprise an olefin.
24. The composition of any one of embodiments 19-23, wherein both R₂₂ are the same.
25. The composition of any one of embodiments 19-24, wherein each R₂₂ is independently

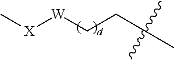

optionally substituted with one or more methyl, wherein
d is 0-7;
W is a bioreducible and/or hydrolysable group; and
X is —(CH₂)_{a′}— wherein a′ is 8-15 and a′+d is 14-22; or
X is —(CH₂)ₐ—CH═CH—(CH₂)_{c}, wherein a is 5-12, c is 2-8 and wherein a+c is 7-15 and a+c+d=12-22;
provided that when n is 2, W is —O—(C═O)—, d is 4, and X is —(CH₂)ₐ—, then a′ is not 10.
26. The composition of embodiment 25, wherein the ionizable lipid is a compound of the formula (I-A):

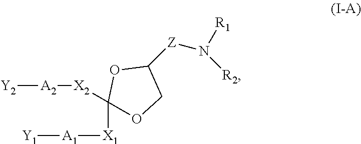

wherein
   Z is —(CH₂)ₙ—, wherein n is 2, 3 or 4;
   R₁ and R₂ are each C₁₋₄ alkyl optionally substituted with hydroxyl;
   R₂₂ is —X₁-A₁-Y₁ or —X₂-A₂-Y₂;
   X₁ and X₂ are each independently —(CH₂)—(CH₂)_{d}— wherein d is 0-7;
   W is A₁ or A₂ and each A₁ and A₂ is independently —O—(C═O)— or —(C═O)—O—;
   CH₃—X— is Y₁ or Y₂, and Y₁ and Y₂ are each independently linear alkyl or alkenyl hydrocarbon chains that are each (15-d) to (24-d) carbon atoms in length and each contain up to one olefin.
27. The composition of embodiment 26, wherein d is 7 in X₁ and X₂, and Y₁ and Y₂ are each linear hydrocarbon chains having 10-12 total carbon atoms and up to one olefin.
28. The composition of any one of embodiments 25-26, wherein d is 5.
29. The composition of any one of embodiments 25-26, wherein d is 6.
30. The composition of any one of embodiments 25-26, wherein d is 7.
31. The composition of any one of embodiments 25-26, wherein d is 7, a′ is 9-11 and a+c is 7-9.
32. A lipid nanoparticle (LNP) composition comprising an ionizable lipid having a chemical structure

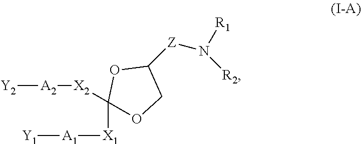

wherein
   Z is —(CH₂)ₙ—, wherein n is 2, 3 or 4;
   R₁ and R₂ are each C₁₋₄ alkyl optionally substituted with one or more hydroxyl;
   -A₁- and -A₂- are each hydrolysable or bioreducible groups;
   —X₁— and —X₂— are each linear hydrocarbon alkyl chains optionally having one or more methyl side groups and having a total number of (a) carbon atoms including any methyl side groups; and
   —Y₁— and —Y₂— are each linear hydrocarbon chains optionally comprising an olefin, optionally having one or more methyl side groups, and having a total number of (b) carbon atoms, including any methyl side groups;
   wherein (a)+(b) is 11-25 and the ratio of (a)/(b) is selected according to the following relationships:

| (a) carbon atoms | (b) carbon atoms |
|---|---|
| in X1 and X2 | in Y1 and Y2 |
| 3 | 16 or more |
| 4 | 14 or more |
| 5 | 12 or more |
| 6 | 9 or more |
| 7 | 7 or more |
| 8 | 4 or more |
| 9 | 2 or more |
| 10 or more | 1 or more |

33. The composition of embodiment 32, wherein R₁ and R₂ are each methyl, and wherein the ratio of (a)/(b) is selected according to the following relationships:

| (a) carbon atoms in X1 and X2 | (b) carbon atoms in Y1 and Y2 |
|---|---|
| 3 | 22 or more |
| 4 | 19 or more |
| 5 | 15 or more |
| 6 | 12 or more |
| 7 | 9 or more |
| 8 | 6 or more |
| 9 | 3 or more |
| 10 or more | 1 or more |

34. The composition of any one of embodiments 32-33, wherein -A₁- and -A₂- are the same.
35. The composition of any one of embodiments 32-34, wherein -A₁- and -A₂- are each esters.
36. The composition of any one of embodiments 32-35, wherein —X₁— and —X₂— are the same.
37. The composition of any one of embodiments 32-36, wherein —X₁— and —X₂— are each independently —(CH₂)ₐ— and a is 3-10.
38. The composition of any one of embodiments 32-36, wherein —X₁— and —X₂— are each independently —(CH₂)ₐ— and a is 6-8.
39. The composition of any one of embodiments 32-38, wherein —Y₁— and —Y₂— are the same.
40. The composition of any one of embodiments 32-39, wherein —Y₁— and —Y₂— are linear saturated hydrocarbon chains.
41. The composition of any one of embodiments 32-40, wherein —Y₁— and —Y₂— comprise a single olefin.
42. The composition of any one of embodiments 32-41, wherein —X₁— and —X₂— are each independently —(CH₂)ₐ— and —Y₁— and —Y₂— are each —(CH₂)_{b}—CH═CH—(CH₂)_{b′}—H wherein a and b are each at least 3 and a+b+b′ is 16-24.
43. The composition of embodiment 32, wherein the compound is selected from the list of compounds in the following Table:

| Name | R1, R2 | Z | X1, X2 | A1, A2 | Y1, Y2 |
|---|---|---|---|---|---|
| KC2-X1-G | —CH3 | —(CH₂)₂— | —(CH₂)₇— | —C(═O)—O— | —(CH₂)₁₁—H |
| KC2-X1-H | —CH3 | —(CH₂)₂— | —(CH₂)₇— | —C(═O)—O— | —(CH₂)₂—CH═CH—(CH₂)₇—H |
| KC2-X1-K | —CH3 | —(CH₂)₂— | —(CH₂)₈— | —C(═O)—O— | —(CH₂)₁₂—H |
| KC3-X1-E | —CH3 | —(CH₂)₃— | —(CH₂)₆— | —C(═O)—O— | —(CH₂)₂—CH═CH—(CH₂)₈—H |
| KC3-X1-F | —CH3 | —(CH₂)₃— | —(CH₂)₆— | —C(═O)—O— | —(CH₂)₂—CH═CH—(CH₂)₁₀—H |
| KC3-X1-G | —CH3 | —(CH₂)₃— | —(CH₂)₇— | —C(═O)—O— | —(CH₂)₁₁—H |
| KC3-X1-H | —CH3 | —(CH₂)₃— | —(CH₂)₇— | —C(═O)—O— | —(CH₂)₂—CH═CH—(CH₂)₇—H |
| KC3-X1-I | —CH3 | —(CH₂)₃— | —(CH₂)₇— | —C(═O)—O— | —(CH₂)₂—CH═CH—(CH₂)₉—H |
| KC3-X1-J | —CH3 | —(CH₂)₃— | —(CH₂)₇— | —C(═O)—O— | —(CH₂)₁₂—H |
| KC3-X1-K | —CH3 | —(CH₂)₃— | —(CH₂)₈— | —C(═O)—O— | —(CH₂)₁₂—H |
| KC3-X1-L | —CH3 | —(CH₂)₃— | —(CH₂)₈— | —C(═O)—O— | —(CH₂)₁₀—H |
| KC3-X1-M | —CH3 | —(CH₂)₃— | —(CH₂)₈— | —C(═O)—O— | —(CH₂)₁₁—H |
| KC3-X1-N | —CH3 | —(CH₂)₃— | —(CH₂)₅— | —C(═O)—O— | —(CH₂)₁₃—H |
| KC3-X1-O | —CH3 | —(CH₂)₃— | —(CH₂)₅— | —C(═O)—O— | —(CH₂)₁₅—H |
| KC3-X1-P | —CH3 | —(CH₂)₃— | —(CH₂)₄— | —C(═O)—O— | —(CH₂)₁₄—H |
| KC3-X1-Q | —CH3 | —(CH₂)₃— | —(CH₂)₄— | —C(═O)—O— | —(CH₂)₁₆—H |
| KC3-X1-R | —CH3 | —(CH₂)₃— | —(CH₂)₈— | —C(═O)—O— | —(CH₂)₁₄—H |
| KC3-X1-S | —CH3 | —(CH₂)₃— | —(CH₂)₈— | —C(═O)—O— | —(CH₂)₂—CH═CH—(CH₂)₈—H |
| KC3-X1-T | —CH3 | —(CH₂)₃— | —(CH₂)₈— | —C(═O)—O— | —(CH₂)₁₆—H |
| KC3-X1-U | —CH3 | —(CH₂)₃— | —(CH₂)₁₀— | —C(═O)—O— | —(CH₂)₁₀—H |
| KC3-X1-V | —CH3 | —(CH₂)₃— | —(CH₂)₁₀— | —C(═O)—O— | —(CH₂)₁₂—H |
| KC3-X2-G | —CH3 | —(CH₂)₃— | —(CH₂)₇— | —O—C(═O)— | —(CH₂)₁₁—H |
| KC3-X2-K | —CH3 | —(CH₂)₃— | —(CH₂)₈— | —O—C(═O)— | —(CH₂)₁₂—H |
| KC3-X2-R | —CH3 | —(CH₂)₃— | —(CH₂)₈— | —O—C(═O)— | —(CH₂)₁₄—H |
| KC4-X1-H | —CH3 | —(CH₂)₄— | —(CH₂)₇— | —C(═O)—O— | —(CH₂)₂—CH═CH—(CH₂)₇—H |

44. The composition of embodiment 19, wherein each R₂₂ is a phytyl group.
45. The composition of embodiment 32, wherein the compound is selected from the list of compounds in the following Table:

| Name | R1, R2 | Z | X1, X2 | A1, A2 | Y1, Y2 |
|---|---|---|---|---|---|
| KC2-X1- | —CH3 | —(CH₂)₂— | —(CH₂)₆— | —C(═O)—O— | —(CH₂—CH₂—CH(—CH₃)—CH₂)₃—H |
| PH1 | | | | | |
| KC3-X1- | —CH3 | —(CH₂)₃— | —(CH₂)₆— | —C(═O)—O— | —(CH₂—CH₂—CH(—CH₃)—CH₂)₃—H |
| PH1 | | | | | |
| KC3-X1- | —CH3 | —(CH₂)₃— | —(CH₂)₆— | —C(═O)—O— | —(CH₂—CH═C(—CH₃)—CH₂)₃—H |
| PH1-B | | | | | |
| KC4-X1- | —CH3 | —(CH₂)₄— | —(CH₂)₆— | —C(═O)—O— | —(CH₂—CH₂—CH(—CH₃)—CH₂)₃—H |
| PH1 | | | | | |
| KC3-X1- | —CH3 | —(CH₂)₃— | —(CH₂)₆— | —C(═O)—O— | —(CH₂—CH₂—CH(—CH₃)—CH₂)₂—CH₂—CH₃ |
| PH2 | | | | | |
| KC3-X1- | —CH3 | —(CH₂)₃— | —(CH₂)₆— | —C(═O)—O— | —(CH₂—CH₂—CH(—CH₃)—CH₂—CH₂—CH═C(—CH₃)—CH₂—CH₂)—H |
| PH2-B | | | | | |
| KC3-X1- | —CH3 | —(CH₂)₃— | —(CH₂)₄— | —C(═O)—O— | —(CH₂—CH₂—CH(—CH₃)—CH₂)₃—H |
| PH3 | | | | | |
| KC3-X1- | —CH3 | —(CH₂)₃— | —(CH₂)₄— | —C(═O)—O— | —(CH₂—CH═C(—CH₃)—CH₂)₃—H |
| PH3-B | | | | | |
| KC3-X1- | —CH3 | —(CH₂)₃— | —(CH₂)₄— | —C(═O)—O— | —(CH₂—CH₂—CH(—CH₃)—CH₂)₄—H |
| PH4 | | | | | |
| KC3-X1- | —CH3 | —(CH₂)₃— | —(CH₂)₄— | —C(═O)—O— | —(CH₂—CH═C(—CH₃)—CH₂)₄—H |
| PH4-B | | | | | |
| KC2-X1- | —CH3 | —(CH₂)₂— | —(CH₂)₇— | —C(═O)—O— | —(CH₂—CH₂—CH(—CH₃)—CH₂)₃—H |
| PH5 | | | | | |
| KC3-X1- | —CH3 | —(CH₂)₃— | —(CH₂)₇— | —C(═O)—O— | —(CH₂—CH₂—CH(—CH₃)—CH₂)₃—H |
| PH5 | | | | | |
| KC3-X1- | —CH3 | —(CH₂)₃— | —(CH₂)₇— | —C(═O)—O— | —(CH₂—CH═C(—CH₃)—CH₂)₃—H |
| PH5-B | | | | | |
| KC2-X1- | —CH3 | —(CH₂)₂— | —(CH₂)₈— | —C(═O)—O— | —(CH₂—CH₂—CH(—CH₃)—CH₂)₃—H |
| PH6 | | | | | |
| KC3-X1- | —CH3 | —(CH₂)₃— | —(CH₂)₈— | —C(═O)—O— | —CH₂—CH₂—(CH₂—CH₂—CH(—CH₃)—CH₂)₂—H |
| PH7 | | | | | |
| KC3-X1- | —CH3 | —(CH₂)₂— | —(CH₂)₁₀— | —C(═O)—O— | —(CH₂—CH₂—CH(—CH₃)—CH₂)₃—H |
| PH8 | | | | | |
| KC3-X1- | —CH3 | —(CH₂)₂— | —(CH₂)₈— | —C(═O)—O— | —(CH₂—CH₂—CH(—CH₃)—CH₂)₂—H |
| PH9 | | | | | |
| KC3-X1- | —CH3 | —(CH₂)₂— | —(CH₂)₁₀— | —C(═O)—O— | —(CH₂—CH₂—CH(—CH₃)—CH₂)₂—H |
| PH10 | | | | | |
| KC3-X1- | —CH3 | —(CH₂)₃— | —(CH₂)₈— | —C(═O)—O— | —CH₂—CH₂—(CH₂—CH₂—CH(—CH₃)—CH₂)₃—H |
| PH11 | | | | | |
| KC3-X1- | —CH3 | —(CH₂)₃— | —(CH₂)₁₀— | —C(═O)—O— | —CH₂—CH₂—(CH₂—CH₂—CH(—CH₃)—CH₂)₂—H |
| PH12 | | | | | |

46. The composition of embodiment 32, wherein the compound is:

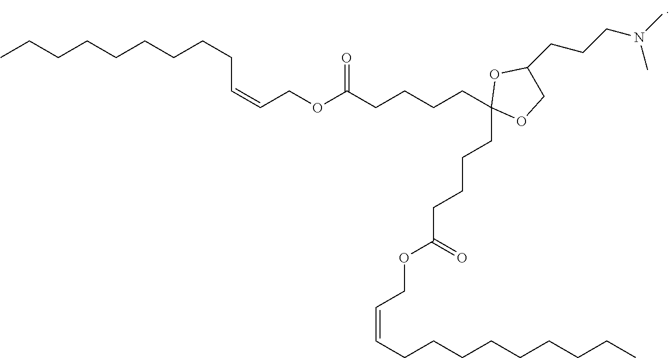 [SMILES: CCCCCCCCC/C=C\COC(=O)CCCCC1(CCCCC(=O)OC/C=C\CCCCCCCCC)OCC(CCCN(C)C)O1]

47. The composition of embodiment 32, wherein the compound is:

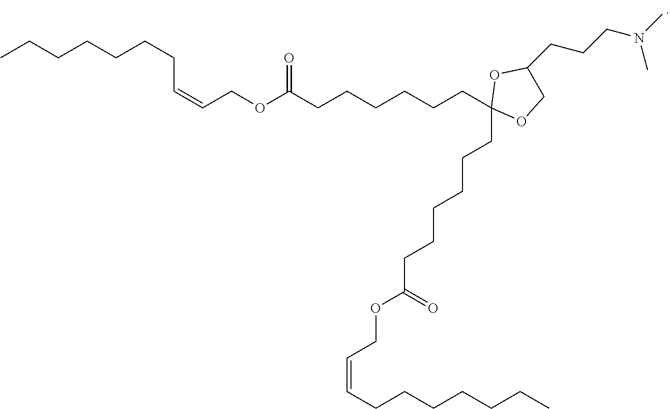 [SMILES: CCCCCCC/C=C\COC(=O)CCCCCCC1(CCCCCCC(=O)OC/C=C\CCCCCCC)OCC(CCCN(C)C)O1]

48. The composition of embodiment 32, wherein the compound is

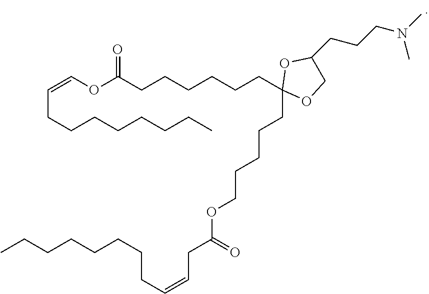 [SMILES: CCCCCCCC/C=C\CC(=O)OCCCCCC1(CCCCCCC(=O)O/C=C\CCCCCCCC)OCC(CCCN(C)C)O1]

49. The composition of embodiment 32, wherein the compound is KC3-X1-G.
50. The composition of embodiment 32, wherein the compound is KC3-X1-H.
51. The composition of embodiment 32, wherein the compound is KC3-X1-K.
52. The composition of embodiment 32, wherein the compound is KC3-X1-L.
53. The composition of embodiment 32, wherein the compound is KC3-X1-M.
54. The composition of embodiment 32, wherein the compound is KC3-X1-R.
55. The composition of embodiment 32, wherein the compound is KC3-X1-S.
56. The composition of embodiment 32, wherein the compound is KC3-X1-T.
57. The composition of embodiment 32, wherein the compound is KC3-X1-U.
58. The composition of embodiment 32, wherein the compound is KC3-X1-V.
59. The composition of embodiment 32, wherein the compound is KC2-X1-K.
60. The composition of embodiment 32, wherein the compound is KC3-X1-PH1.
61. The composition of embodiment 32, wherein the compound is KC3-X1-PH4.
62. The composition of embodiment 32, wherein the compound is KC2-X1-P3H5′.
63. The composition of embodiment 32, wherein the compound is KC3-X1-PH5.
64. The composition of embodiment 32, wherein the compound is KC3-X1-PH5-B.
65. The composition of embodiment 32, wherein the compound is KC2-X1-PH6.
66. The composition of embodiment 32, wherein the compound is KC3-X1-PH7.
67. The composition of embodiment 32, wherein the compound is KC3-X1-PH8.
68. The composition of embodiment 32, wherein the compound is KC3-X1-PH9.
69. The composition of embodiment 32, wherein the compound is KC3-X1-PH110.
70. The composition of embodiment 32, wherein the compound is KC3-X1-PH11.
71. The composition of embodiment 32, wherein the compound is KC3-X1-PH12.
72. The composition of embodiment 32, wherein the compound is KC3-OA:

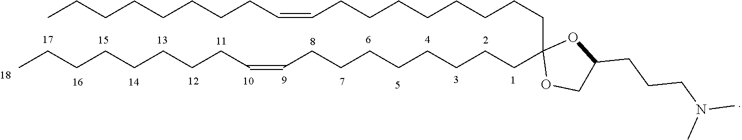 [SMILES: CCCCCCCC/C=C\CCCCCCCCC1(CCCCCCCC/C=C\CCCCCCCC)OC[C@H](CCCN(C)C)O1]

73. The composition of embodiment 32, wherein the compound is KC3-OA-A:

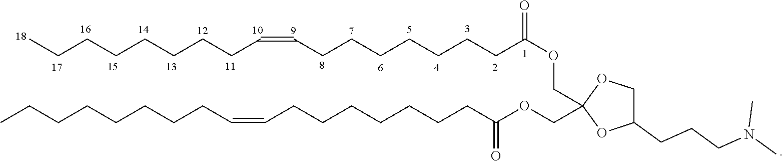 [SMILES: CCCCCCCC/C=C\CCCCCCCC(=O)OCC1(COC(=O)CCCCCCC/C=C\CCCCCCCC)OCC(CCCN(C)C)O1]

74. The composition of embodiment 32, wherein the compound is KC3-OA-B:

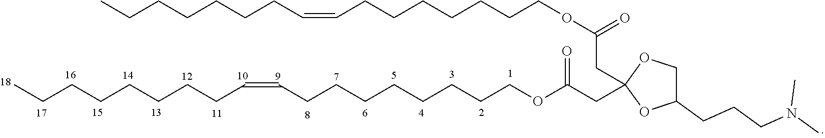 [SMILES: CCCCCCC/C=C\CCCCCCCOC(=O)CC1(CC(=O)OCCCCCCCC/C=C\CCCCCCCC)OCC(CCCN(C)C)O1]

75. The composition of embodiment 32, wherein the compound is KC3-OA-C:

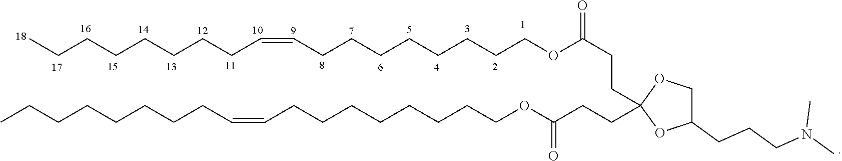 [SMILES: CCCCCCCC/C=C\CCCCCCCCOC(=O)CCC1(CCC(=O)OCCCCCCCC/C=C\CCCCCCCC)OCC(CCCN(C)C)O1]

76. The composition of embodiment 32, wherein the compound is KC4-OA-C:

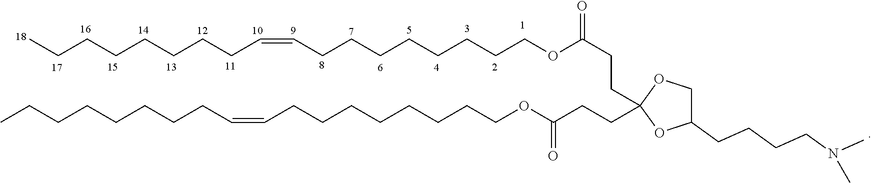 [SMILES: CCCCCCCC/C=C\CCCCCCCCOC(=O)CCC1(CCC(=O)OCCCCCCCC/C=C\CCCCCCCC)OCC(CCCCN(C)C)O1]

77. The composition of embodiment 32, wherein the compound is KC2-OA-C:

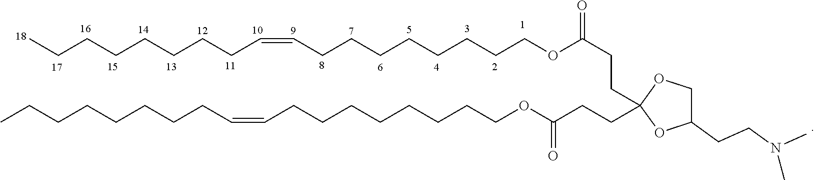 [SMILES: CCCCCCCC/C=C\CCCCCCCCOC(=O)CCC1(CCC(=O)OCCCCCCCC/C=C\CCCCCCCC)OCC(CCN(C)C)O1]

78. The composition of embodiment 32, wherein the compound is 4-1:

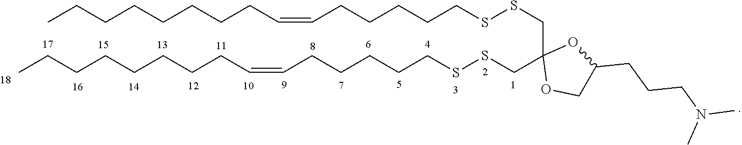 [SMILES: CCCCCCCC/C=C\CCCCCSSCC1(CSSCCCCC/C=C\CCCCCCCC)OCC(CCCN(C)C)O1]

79. The composition of embodiment 32, wherein the compound is 4-2:

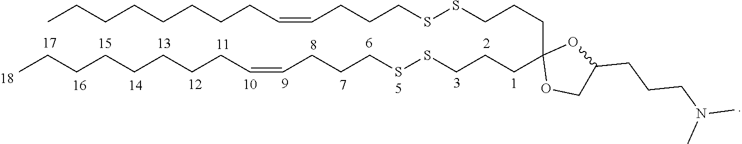 [SMILES: CCCCCCCC/C=C\CCCSSCCCC1(CCCSSCCC/C=C\CCCCCCCC)OCC(CCCN(C)C)O1]

80. A lipid nanoparticle (LNP) vaccine composition comprising:
   a. a nucleic acid;
   b. an ionizable cationic lipid at a N/P ratio of 3 to 8 relative to the nucleic acid, wherein the ionizable lipid has the chemical structure of any one of embodiments 1-79 and
      wherein the ionizable cationic lipid is present in the LNP vaccine composition in a total amount of 40-65 mol % of the total lipid content of the LNP composition;
   c. a sterol in a total amount of 25-45 mol % of the total lipid content of the LNP composition;
   d. one or more phospholipids in a total amount of phospholipids of 5-25 mol % of the total lipid content of the LNP composition; and
   e. a conjugated lipid in a total amount of 0.5-2.5 mol % of the total lipid content of the LNP composition.
81. The composition of embodiment 80, wherein the one or more phospholipids comprises a phosphatidylserine (PS) lipid in a total amount of 2.5-10 mol % of the total lipid content of the LNP composition.
82. The composition of embodiment 81, wherein the PS lipid is DSPS or DPPS.
83. The composition of embodiment 80, wherein a second phospholipid is selected from the group consisting of: DSPC, HSPC, DPPC, and sphingomyelin.
84. The composition of any one of embodiments 80-83, having an N/P ratio of 5-6 relative to the nucleic acid.
85. The composition of embodiment 84, wherein the nucleic acid is an RNA.
86. The composition of embodiment 84, wherein the nucleic acid is mRNA, optionally chemically modified RNA, and further optionally modified with N-methylpseudouridine.
87. A lipid nanoparticle (LNP) composition comprising:
   a. a nucleic acid;
   b. an ionizable cationic lipid at a N/P ratio of 3 to 8 relative to the nucleic acid wherein the ionizable lipid has the chemical structure of any one of embodiments 1-79, and wherein the ionizable cationic lipid is present in the LNP composition in a total amount of 40-65 mol % of the total lipid content of the LNP composition;
   c. a sterol in a total amount of 25-45 mol % of the total lipid content of the LNP composition;
   d. one or more phospholipids in a total amount of phospholipids of 5-25 mol % of the total lipid content of the LNP composition, and comprising a dipalmitoylphosphatidylserine (DPPS) lipid in a total amount of 2.5-10 mol % of the total lipid content of the LNP composition; and
   e. a conjugated lipid in a total amount of 0.5-2.5 mol % of the total lipid content of the LNP composition.
88. The composition of embodiment 87, wherein a second phospholipid is selected from the group consisting of. DSPC, HSPC, and sphingomyelin.
89. The composition of any one of embodiments 87-88, wherein the ionizable cationic lipid is at an N/P ratio of 5-6 relative to the nucleic acid.
90. The composition of any one of embodiments 87-89, wherein the nucleic acid is an mRNA.
91. The composition of embodiment 90, wherein the mRNA is chemically modified with N-nethylpseudouri dine.
92. A nucleic acid lipid nanoparticle (LNP) composition comprising:
   a. a nucleic acid;
   b. an ionizable cationic lipid of any one of embodiments 1-79, at a N/P ratio of 3 to 8 relative to the nucleic acid in a total amount of 40-65 mol % of the total lipid content of the LNP composition;
   c. a sterol in a total amount of 25-45 mol % of the total lipid content of the LNP composition;
   d. d. one or more phospholipids in a total amount of phospholipids of 5-25 mol % of the total lipid content of the LNP composition; and
   e. a conjugated lipid in a total amount of 0.5-2.5 mol % of the total lipid content of the LNP composition comprising a PEG-lipid having a poly(ethylene glycol) chain terminally attached to a linking moiety and two hydrocarbon chains terminally attached to the same linking moiety, wherein the hydrocarbon chains are saturated C₁₂-chains independently selected from n-dodecyl (lauryl) group and n-dodecanoyl (lauroyl) group.
93. The composition of embodiment 92, wherein the linking moiety is glyceryl group, N-oxycarbonyl glycerophoshoryl ethanolaminocarbonyl group, oxycarbonylamide group, or oxyacetamide group.
94. The composition of embodiment 93, wherein the poly(ethyene glycol) chain is methoxy-poly(ethyene glycol) with an average molecular weight of 2000.
95. The composition of embodiment 94, wherein the PEG-lipid is mPEG-1,2-dilauroylglycerol (PEG-DLG), mPEG-1,2-dilaurylglycerol (PEG-DLG), PEG-1,2-dilaurylglycerol, PEG-DLPE, PEG-oxycarbonyl-N,N-didodecylamide, or mPEG-N,N-didodecylacetamide.
96. The composition of any one of embodiments 92-95, wherein the LNP has an z-average particle size of 60-150 nm and is freeze-thaw stable.
97. The composition of any one of embodiments 94-96, wherein one or more phospholipids comprise a phosphatidylserine (PS) lipid in a total amount of 2.5-10 mol % of the total lipid content of the LNP composition, and wherein the LNP composition comprises a phosphatidylserine (PS) lipid in a total amount of 2.5-10 mol % of the total lipid content of the LNP composition.
98. A nucleic acid lipid nanoparticle (LNP) human vaccine composition comprising:
   a. a nucleic acid;
   b. an ionizable cationic lipid of any one of embodiments 1-79, at a N/P ratio of 3 to 8 relative to the nucleic acid in a total amount of 40-65 mol % of the total lipid content of the LNP composition;
   c. a sterol in a total amount of 25-45 mol % of the total lipid content of the LNP composition;
   d. one or more phospholipids in a total amount of phospholipids of 5-25 mol % of the total lipid content of the LNP composition, and comprising a phosphatidylglycerol (PG) in a total amount of 1.0-10 mol % of the total lipid content of the LNP composition; and e. a conjugated lipid in a total amount of 0.5-2.5 mol % of the total lipid content of the LNP composition.
99. The composition of embodiment 98, wherein
   a. the sterol is cholesterol; and
   b. the phosphatidylglycerol (PG) is an anionic phospholipid selected from the group consisting of: distearoylphosphatidylglycerol (DSPG) and dipalmitoyphosphatidylglycerol (DPPG).
100. The composition of embodiment 99, wherein the nucleic acid is mRNA and the ionizable cationic lipid is present at a N/P ratio of 4 to 7 relative to the nucleic acid.
101. The composition of embodiment 100, wherein the conjugated lipid is selected from PEG-DMG, PEG-DLG, and PEG-DLPE.
102. The composition of embodiment 98, wherein the one or more phospholipids comprise a phospholipid selected from the group consisting of:
   distearoylphosphatidylcholine (DSPC) and hydrogenated soy phosphatidylcholine (HSPC).
103. An LNP composition comprising:
   a. a nucleic acid, wherein the nucleic acid is an mRNA;
   b. a cholesterol sterol in a total amount of 25-40 mol % of the total lipid content of the LNP composition;
   c. an ionizable cationic lipid of any one of embodiments 1-79 having a N/P ratio of 3 to 8 relative to the nucleic acid in a total amount of 40-65 mol % of the total lipid content of the LNP composition;
   d. one or more phospholipids in a total amount of phospholipids of 5-25 mol % of the total lipid content of the LNP composition, the one or more phospholipids are selected from the group consisting of: DSPC, HSPC, DPPC, and sphingomyelin,
   e. an ammonium salt of dipalmitoylphosphatidyl-L-serine ((L-serine)DPPS) lipid in a total amount of 2.5-10 mol % of the total lipid content of the LNP composition; and
   f. a distearoylphosphatidylcholine (DSPC) phospholipid in a total amount of 5-25 mol % of the total lipid content of the LNP composition; and
   g. a PEG-containing conjugated lipid in a total amount of 0.5-2.5 mol % of the total lipid content of the LNP composition.
104. The composition of any one of embodiments 80-103, wherein the nucleic acid encodes influenza virus hemagglutinin (HA).
105. The composition of embodiment 104, wherein the nucleic acid encodes an influenza H5 influenza virus antigen.
106. The composition of embodiment 104, wherein the nucleic acid encodes an influenza H5N1 influenza virus antigen.
107. The composition of embodiment 104, wherein the nucleic acid is mRNA comprising SEQ ID NO:5, or encoding a polypeptide encoded by SEQ ID NO:5.
108. The composition of embodiment 104, wherein the nucleic acid is mRNA comprising SEQ ID NO:6, or encoding a polypeptide encoded by SEQ ID NO:6.
109. The composition of embodiment 104, wherein the nucleic acid is mRNA comprising SEQ ID NO:7, or encoding a polypeptide encoded by SEQ ID NO:7.
110. The composition of embodiment 104, wherein the nucleic acid is mRNA comprising SEQ ID NO:7, or encoding a polypeptide encoded by SEQ ID NO:8.
111. The composition of any one of embodiments 80-103, wherein the nucleic acid encodes a respiratory syncytial virus (RSV) fusion glycoprotein.
112. The composition of embodiment 111, wherein the nucleic acid encodes a respiratory syncytial virus (RSV) A2 fusion glycoprotein.
113. The composition of embodiment 111, wherein the nucleic acid encodes a polypeptide sequence of SEQ ID NO:9, or comprises a polynucleotide sequence of SEQ ID NO:10.
114. The composition of any one of embodiments 1-113, wherein the composition comprises a biodegradable KC3-X1 ionizable cationic lipid.
115. The composition of any one of embodiments 1-113, wherein the composition comprises a biodegradable KC3-X2 ionizable cationic lipid.
116. The composition of any one of embodiments 1-113, wherein the composition comprises a biodegradable KC3-OA ionizable cationic lipid.
117. The composition of any one of embodiments 1-116, wherein the composition comprises 48-54 mol % of the ionizable cationic lipid.
118. The composition of embodiment 117, comprising 48 mol % of the ionizable cationic lipid.
119. The composition of embodiment 117, comprising 50 mol % of the ionizable cationic lipid.
120. The composition of embodiment 117, comprising 52 mol % of the ionizable cationic lipid.
121. The composition of embodiment 117, comprising 54 mol % of the ionizable cationic lipid.
122. The composition of any one of embodiments 1-121, comprising 5 mol % DPPS.
123. A method for administering a nucleic acid to a subject in need thereof comprising administering a nucleic acid lipid nanoparticle (LNP) composition to a human subject, the nucleic acid LNP composition comprising:
   a. a nucleic acid;
   b. an ionizable cationic lipid of any one of embodiments 1-79 at a N/P ratio of 3 to 8 relative to the nucleic acid in a total amount of 40-65 mol % of the total lipid content of the LNP composition;
   c. a sterol in a total amount of 25-45 mol % of the total lipid content of the LNP composition;
   d. one or more phospholipids in a total amount of phospholipids of 5-25 mol % of the total lipid content of the LNP composition, and comprising a phosphatidylglycerol (PG) in a total amount of 1.0-10 mol % of the total lipid content of the LNP composition; and
   e. a conjugated lipid in a total amount of 0.5-2.5 mol % of the total lipid content of the LNP composition.
124. A method for vaccinating a subject in need thereof, comprising administering to the subject a therapeutically effective amount of a vaccine comprising an ionizable cationic lipid of any one of embodiments 1-79.
125. The method of embodiment 124, wherein the vaccine further comprises a lipid nanoparticle composition of any one of embodiments 1-134.
126. The method of embodiment 125, wherein the vaccine comprises a nucleic acid encoding an antigen.
127. The method of embodiment 125, wherein the antigen is an influenza antigen or a RSV antigen.
128. The method of embodiment 125, wherein the nucleic acid encodes an antigen listed in Table 12.
129. The method of any one of embodiments 126-128, wherein the nucleic acid is a polynucleotide sequence comprising any one of SEQ ID NOs: 4, 6, 8, or 10, or a polynucleotide encoding a polypeptide sequence of SEQ ID NOs: 5, 7, or 9.
130. A composition comprising an ionizable cationic lipid compound consisting of a 1,3-dioxolane ring covalently bound to both (a) an alkylamine or alkylhydroxy head group, and (b) a pair of geminal functionalized hydrocarbon tail chains each comprising a linear hydrocarbon chain extending from the 1,3-dioxalane ring to an ester functional group, and one or more isoprenoid or isoprene moieties distal to the ester functional group from the 1,3-dioxolane ring.
131. The composition of embodiment 130, wherein the hydrocarbon tail chains are each phytyl groups optionally comprising one or more olefins.
132. The composition of embodiment 131, wherein the ionizable cationic lipid compound consists of a 1,3-dioxolane ring covalently bound to both (a) an alkylamine head group having a pKa of 6-7.5, and (b) a pair of identical 20-24 atom functionalized aliphatic chains each comprising the ester functional group positioned at least 7 atoms from the 1,3-dioxalane ring.
133. The composition of embodiment 130, wherein the ionizable cationic lipid is a compound of Formula (I):

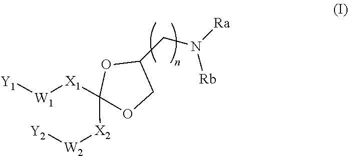

wherein
   Y₁ and Y₂ in Formula (I-A) are each independently is a C₄-C₁₅ aliphatic hydrocarbon comprising two or more isoprene or isoprenoid moieties.
   W₁ and W₂ are each independently an ester or disulfide functional group; and
   X₁ and X₂ are each independently *—(CH₂)_{d}—(CH₂)—**, wherein d is an integer from 2-11 (including 2 and 11), provided that (a+d) is an integer less than 23, (b+c+d) is an integer less than 21 and (p+q+d) is an integer less than 16;
   Rₐ is methyl or C₂-C₄ alkyl optionally substituted with hydroxyl; and
   R_{b} is methyl.
134. The composition of embodiment 133, wherein Y₁ and Y₂ are each independently -(A₁)ₐ-A₂ and a is 1-3, wherein:
   a) each A₁ is independently —(CH₂)_{b}— wherein b is 1-4; —(CH₂)₂—C(CH₃)—CH₂—, and —(CH₂)—CH═C(CH₃)—CH₂—(Z); and
   b) A₂ is *—(CH₂)₂—C(CH₃)₂, *—(CH₂)—CH═C(CH₃)₂ (Z).
135. A composition comprising a compound consisting of a 1,3-dioxolane ring covalently bound to both (a) an ionizable C₂-C₆ alkylamine head group, and (b) a pair of geminal and linear 15-26 atom functionalized aliphatic chains each comprising an ester functional group positioned at least 7 atoms from the 1,3-dioxolane ring and each of the aliphatic chains optionally comprising one or two olefin groups.
136. The composition of embodiment 135, wherein the compound is an ionizable cationic lipid compound consists of a 1,3-dioxolane ring covalently bound to (a) an alkylamine head group selected from —(CH₂)₂₋₄—N(CH₃)₂, and —(CH₂)₂₋₄—N(CH₃)—(CH₂)₂₋₄—OH, and (b) a pair of identical 20-24 atom functionalized aliphatic chains each consisting of an ester functional group positioned 7-10 atoms from the 1,3-dioxolane ring and each aliphatic chain having a single (Z) olefin group positioned 3 atoms from the ester group.
137. The use of a KCn ionizable cationic lipid in an ionizable cationic lipid (LNP) pharmaceutical composition, the KCn Ionizable Cationic Lipid comprising a head group having an ionizable amine with a pKa of 6-7.5, and a pair of geminal 20-26 carbon aliphatic hydrocarbon chains each comprising an ester group and a linear 7-10 carbon saturated aliphatic hydrocarbon chain between the ester group and the head group.

In some embodiments, the ionizable cationic lipid is a compound of one or more of the following additional embodiments:
Embodiment 1: An ionizable cationic lipid compound comprising a 1,3-dioxolane ring covalently bound to both (a) an alkylamine head group having a pKa of 6-7.5, and (b) a pair of identical geminal functionalized aliphatic chains at the 2-position of the 1,3-dioxlane ring, each having 18-25 total carbon atoms and each functionalized aliphatic chain consisting of (i) a 7-10 carbon linear aliphatic group covalently bound to the 2′-position of the 1,3-dioxolane ring and to (ii) an ester functional group, and (iii) a 10-20 carbon aliphatic chain covalently bound to the ester functional group; or
an ionizable cationic lipid compound comprising a 1,3-dioxolane ring covalently bound to both (a) an alkylamine head group having a pKa of 6-7.5, and (b) a pair of identical geminal functionalized aliphatic chains at the 2-position of the 1,3-dioxlane ring, each having 18-25 total carbon atoms and each functionalized aliphatic chain consisting of (i) a linear aliphatic group covalently bound to the 2′-position of the 1,3-dioxolane ring and to (ii) an ester functional group, and (iii) a carbon branched aliphatic chain covalently bound to the ester functional group and comprising two or more branched C₅ hydrocarbon monomer units each joined to the aliphatic chain at the 1-carbon and 4-carbon positions of each C₅ hydrocarbon monomer unit.
Embodiment 2: The compound of embodiment 1, wherein the compound is a compound of Formula (I):

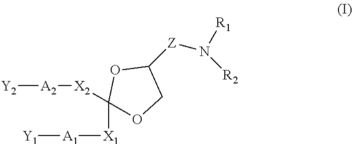

or a pharmaceutically acceptable salt thereof, wherein
   R₁ and R₂ are each independently C₁₋₄ alkyl optionally substituted with hydroxyl;
   Z is *—(CH₂)ₙ—**, wherein * is a covalent bond to the 1,3-dioxolane ring and ** is a covalent bond to —NR₁R₂, and n is 2, 3 or 4;
   —X₁— and —X₂— are the same of different and are each independently —(CH₂)₍ₐ₎—, wherein (a) is 7-10;
   A₁- and -A₂- are the same or different and are each independently ester groups;
   Y₁— and —Y₂— are the same or different and are each is an aliphatic chain having (b) total carbon atoms, optionally comprising one or two olefins and optionally comprising two or more branched C₅ hydrocarbon monomer units, wherein (b) is 10-20; and
   the sum of (a)+(b) is 18-25; or
wherein
   R₁ and R₂ are each independently C₁₋₄ alkyl optionally substituted with hydroxyl;
   Z is *—(CH₂)ₙ—**, wherein * is a covalent bond to the 1,3-dioxolane ring and ** is a covalent bond to —NR₁R₂, and n is 2, 3 or 4;
   —X₁— and —X₂— are the same of different and are each independently —(CH₂)₍ₐ₎—, wherein (a) is 4-10;
   -A₁- and -A₂- are the same or different and are each independently ester groups;
   —Y₁— and —Y₂— are the same or different and are each is an aliphatic chain having (b) total carbon atoms and comprising two or more branched C₅ hydrocarbon monomer units, wherein (b) is 10-20; and
   the sum of (a)+(b) is 18-25.
Embodiment 3: The compound of embodiment 2, wherein *—Z—N(R₁)(R₂) is selected from —(CH₂)₂₋₄—N(CH₃)₂, and —(CH₂)₂₋₄—N(CH₃)—(CH₂)₂₋₄—OH.
Embodiment 4: The compound of embodiment 3, wherein R₁ and R₂ are each methyl.
Embodiment 5: The compound of any one of embodiments 1-4, wherein —X₁— and —X₂— are the same.
Embodiment 6: The compound of any one of embodiments 1-5, wherein —Y₁— and —Y₂— are the same.
Embodiment 7: The compound of any one of embodiments 1-6, wherein -A₁- and -A₂- are the same.
Embodiment 8: The compound of any one of embodiments 1-7, wherein —Y₁— and —Y₂— are each independently —(CH₂)(b>—H, wherein (b) is 10, 11, 12, 13, 14, 15, 16 or 17.
Embodiment 9: The compound of embodiment 8, wherein (a)+(b) is 20, 21, 22, 23, 24 or 25.
Embodiment 10: The compound of embodiment 9, wherein (a)+(b) is 22.
Embodiment 11: The compound of any one of embodiments 1-7, wherein —Y₁— and —Y₂— are each independently an aliphatic chain having (b) total carbon atoms and comprising one olefin, wherein (b) is 10, 11, 12, 13, 14, 15, 16 or 17.
Embodiment 12: The compound of embodiment 11, wherein —Y₁ and —Y₂ are each independently *—(CH₂)₂—CH═CH—(CH₂)(b-4)H, wherein * is a covalent bond to A₁ or A₂.
Embodiment 13: The compound of any one of embodiments 11-12, wherein the sum of (a)+(b) is 20, 21, 22, 23, 24 or 25.
Embodiment 14: The compound of embodiment 13 wherein (a)+(b) is 22.
Embodiment 15: The compound of any one of embodiments 1-7, wherein —Y₁— and —Y₂— are each independently an aliphatic chain having (b) total carbon atoms, comprising two or more branched C₅ hydrocarbon monomer units, wherein (b) is 10, 11, 12, 13, 14, 15, 16 or 17.
Embodiment 16: The compound of embodiment 15, wherein —Y₁— and —Y₂— are each independently an aliphatic chain having (b) total carbon atoms, comprising two or more branched C₅ hydrocarbon monomer units, wherein (b) is 10, 11, 12, 13, 14, 15, 16 or 17.
Embodiment 17: The compound of any one of embodiments 15-16, wherein —Y₁ and —Y₂ are each independently *—(CH₂)₀₋₂—(R_{y})₂₋₄—(CH₂)₀₋₂—H wherein each R_{y} is independently an isopentane (IPN) or a 2-methylbut-2-ene (2 MB) hydrocarbon monomer unit, and * is a covalent bond to A₁ or A₂.
Embodiment 18: The compound of embodiment 17, wherein —Y₁ and —Y₂ are the same or different and are each independently *—(R_{y})₃ or *—(CH₂)₂—(R_{y})₂, wherein * is a covalent bond to -A₁- or -A₂- respectively.
Embodiment 19: The compound of embodiment 18, wherein —Y₁ and —Y₂ are each *—IPN—IPN—IPN, *—(CH₂)₂—IPN—IPN.
Embodiment 20: The compound of any one of embodiments 15-19, wherein the sum of (a)+(b) is 20, 21, 22, 23, 24 or 25.
Embodiment 21: The compound of embodiment 20, wherein the sum of (a)+(b) is 22.
Embodiment 22: The compound of embodiment 1, wherein the compound is KC3-X1-PH5:

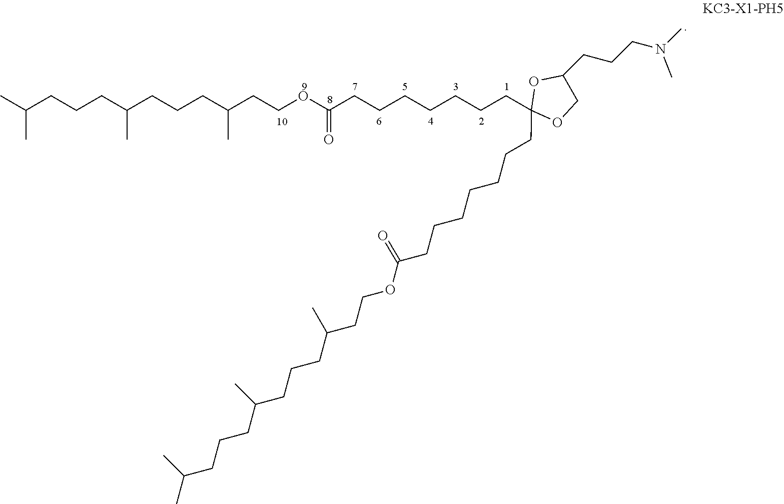 [SMILES: CC(C)CCCC(C)CCCC(C)CCOC(=O)CCCCCCCC1(CCCCCCCC(=O)OCCC(C)CCCC(C)CCCC(C)C)OCC(CCCN(C)C)O1]

Embodiment 23: The compound of embodiment 1, wherein the compound is KC3-X1-PH12:

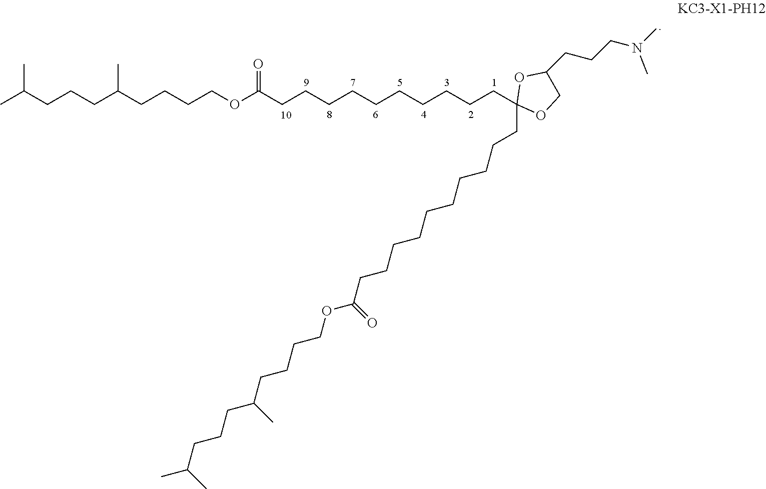 [SMILES: CC(C)CCCC(C)CCCCOC(=O)CCCCCCCCCCC1(CCCCCCCCCCC(=O)OCCCCC(C)CCCC(C)C)OCC(CCCN(C)C)O1]

Embodiment 24: The compound of embodiment 1, wherein the compound is KC3-X2-PH12:
Embodiment 25: The compound of embodiment 1, wherein the compound is KC3-X1-AA:

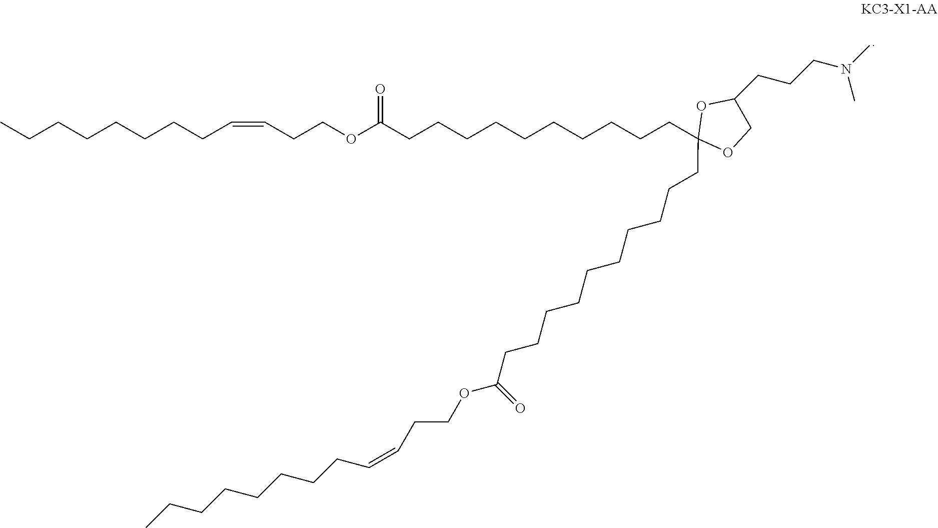 [SMILES: CCCCCCCC/C=C\CCOC(=O)CCCCCCCCCCC1(CCCCCCCCCCC(=O)OCC/C=C\CCCCCCCC)OCC(CCCN(C)C)O1]

Embodiment 26: A lipid nanoparticle (LNP) composition comprising a mRNA nucleic acid encapsulated in lipidic nanoparticles formed from the ionizable cationic lipid of any one of embodiments 1-25, at a N/P ratio of 3 to 8 relative to the mRNA nucleic acid in a total amount of 40-65 mol % of the total lipid content of the LNP composition; a sterol in a total amount of 25-45 mol % of the total lipid content of the LNP composition; one or more phospholipids in a total amount of phospholipids of 5-25 mol % of the total lipid content of the LNP composition; and a PEG-containing conjugated lipid in a total amount of 0.5-5.0 mol % of the total lipid content of the LNP composition.
Embodiment 27: The composition of embodiment 26, wherein the two or more phospholipids comprise a total of 5 mol % of a phosphatidyl-L-serine (PS).
Embodiment 28: The composition of any one of embodiments 26-27, wherein composition comprises the conjugated lipid in a total amount of 0.5-2.5 mol % of the total lipid content of the LNP composition.
Embodiment 29: The composition of any one of embodiments 26-28, wherein
   a. the two or more phospholipids consist of: 1,2-distearoyl-sn-glycero-3-phosphocholine (DSPC) and dipalmitoylphosphatidyl-L-serine (DPPS) or distearoylphosphatidyl-L-serine (DSPS); and
   b. the PEG-containing conjugated lipid in the LNP is PEG(2000)-dimyristoylglycerol (PEG-DMG) or PEG(2000)-dilauroylglycerol (PEG-DLG).
Embodiment 30: The composition of any one of embodiments 26-29, wherein the ionizable cationic lipid is KC3-X1-PH5:

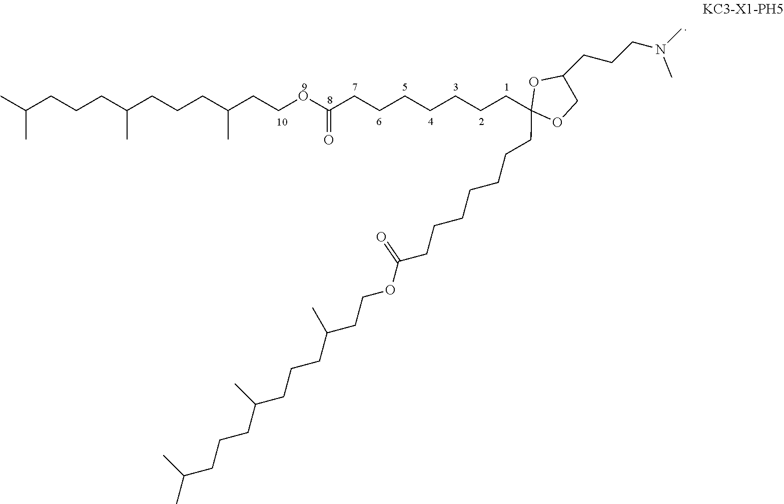 [SMILES: CC(C)CCCC(C)CCCC(C)CCOC(=O)CCCCCCCC1(CCCCCCCC(=O)OCCC(C)CCCC(C)CCCC(C)C)OCC(CCCN(C)C)O1]

Embodiment 31: The composition of any one of embodiments 26-29, wherein the ionizable cationic lipid is KC3-X1-PH12:

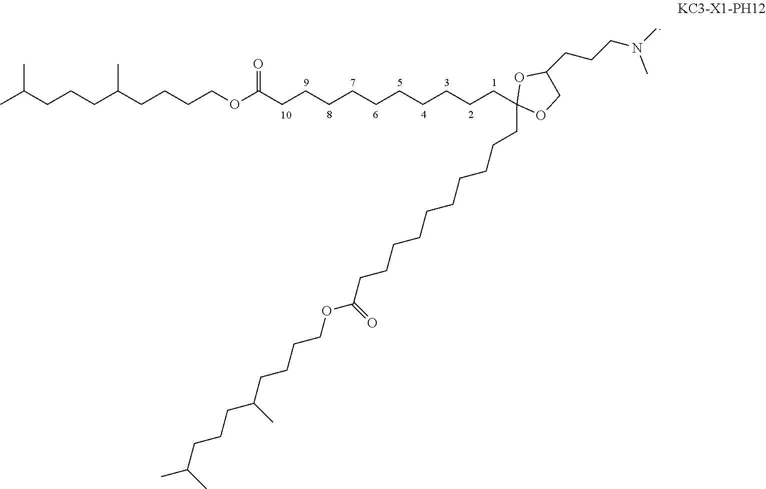 [SMILES: CC(C)CCCC(C)CCCCOC(=O)CCCCCCCCCCC1(CCCCCCCCCCC(=O)OCCCCC(C)CCCC(C)C)OCC(CCCN(C)C)O1]

Embodiment 32: The composition of any one of embodiments 26-29, wherein the ionizable cationic lipid is KC3-X2-PH12:

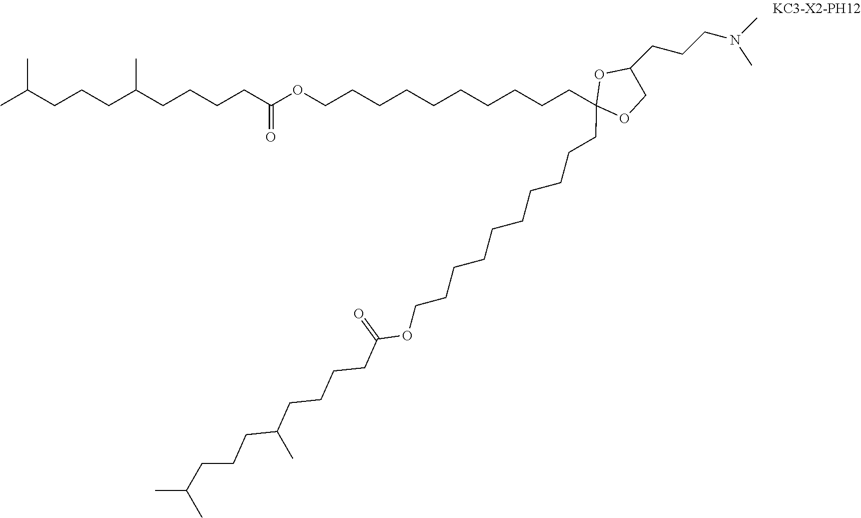 [SMILES: CC(C)CCCC(C)CCCCC(=O)OCCCCCCCCCCC1(CCCCCCCCCCOC(=O)CCCCC(C)CCCC(C)C)OCC(CCCN(C)C)O1]

Embodiment 33: The composition of any one of embodiments 26-29, wherein the ionizable cationic lipid is KC3-X1-AA:

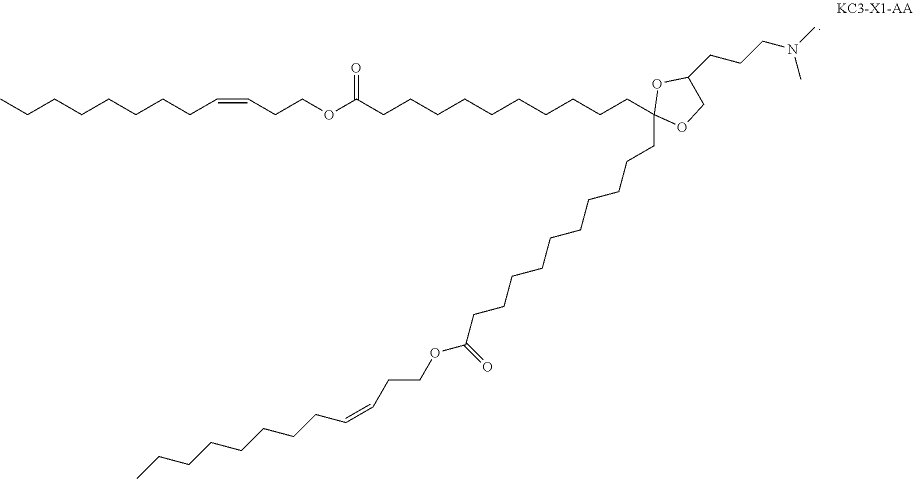 [SMILES: CCCCCCCC/C=C\CCOC(=O)CCCCCCCCCCC1(CCCCCCCCCCC(=O)OCC/C=C\CCCCCCCC)OCC(CCCN(C)C)O1]

### EXAMPLES

While this disclosure has been described in relation to certain embodiments, and many details have been set forth for purposes of illustration, it will be apparent to those skilled in the art that this disclosure includes additional embodiments, and that some of the details described herein may be varied considerably without departing from this disclosure. This disclosure includes such additional embodiments, modifications and equivalents. In particular, this disclosure includes any combination of the features, terms, or elements of the various illustrative components and examples.

Certain examples are provided below to illustrate various embodiments of the embodiments disclosed herein. One of ordinary skill in the art will recognize that the various embodiments disclosed herein are not limited to these specific illustrative examples.

### Synthesis of Ionizable Cationic Lipids of Formula (I).

Ionizable cationic lipids can be prepared, each comprising: (a) a head group comprising an amine having a pKa suitable for the compound to be neutral outside the cell but to acquire a positive charge in the acidic environment of the cellular endosome; and (b) two hydrophobic tails configured to promote self-assembly into lipid nanoparticles and promote membrane fusion or disruption with a cell. An ionizable cationic lipid can include a head group containing a tertiary (or sometimes secondary) amine that can become protonated under acidic conditions. The amine of the head group is selected to have a pKa generally in the range of about 6.0-7.0, allowing the lipid to remain mostly neutral at physiological pH (~7.4) but acquire a positive charge in the acidic environment of the endosome. The chemical structure of the ionizable cationic lipid is selected to provide a head group amine with this pH-responsive for minimizing toxicity in circulation while promoting endosomal escape. The ionizable cationic lipid can also include two hydrophobic tails, often composed of long alkyl chains (saturated or unsaturated), which facilitate self-assembly into lipid nanoparticles and promote membrane fusion or disruption. The length, degree of unsaturation, and branching of these tails strongly influence fluidity and delivery efficiency. Together, the ionizable amine head group and dual hydrophobic tails create an amphiphilic structure optimized for stability in the bloodstream, efficient encapsulation of nucleic acids, and effective intracellular release. The examples provide non-limiting embodiments of ionizable cationic lipids.

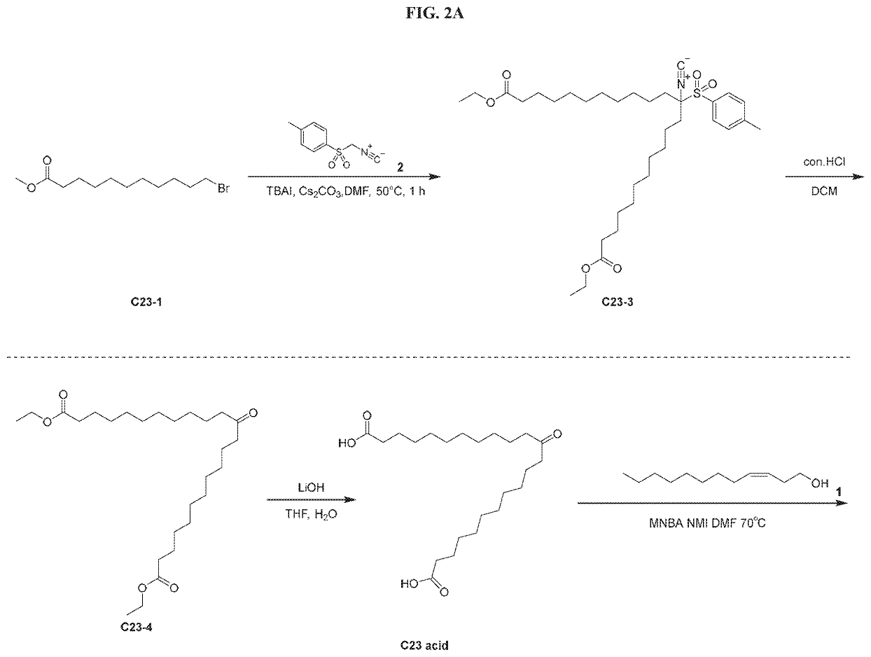

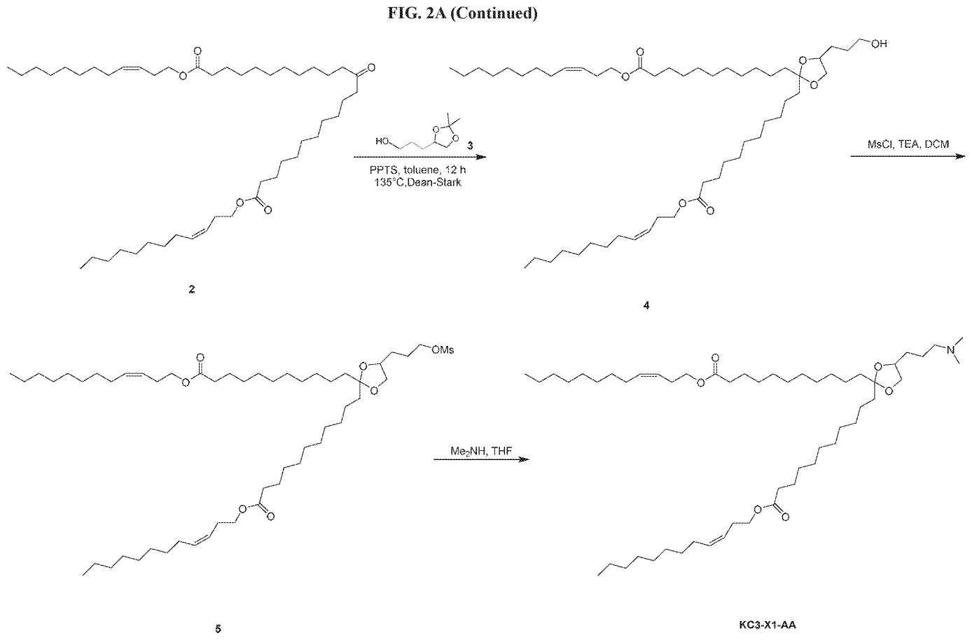

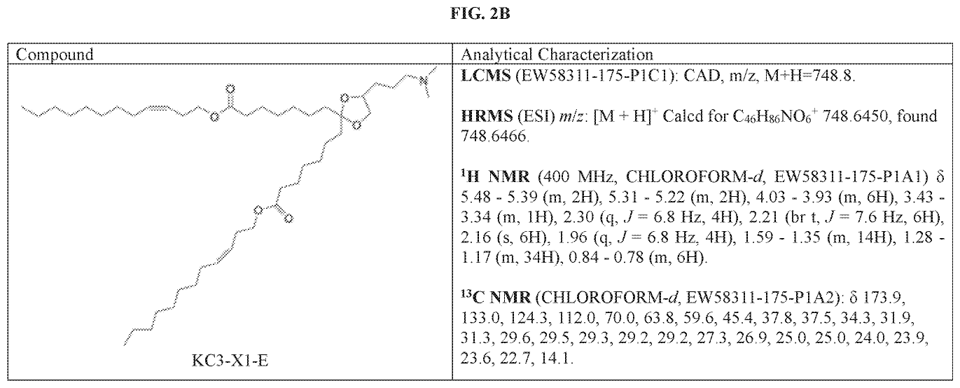

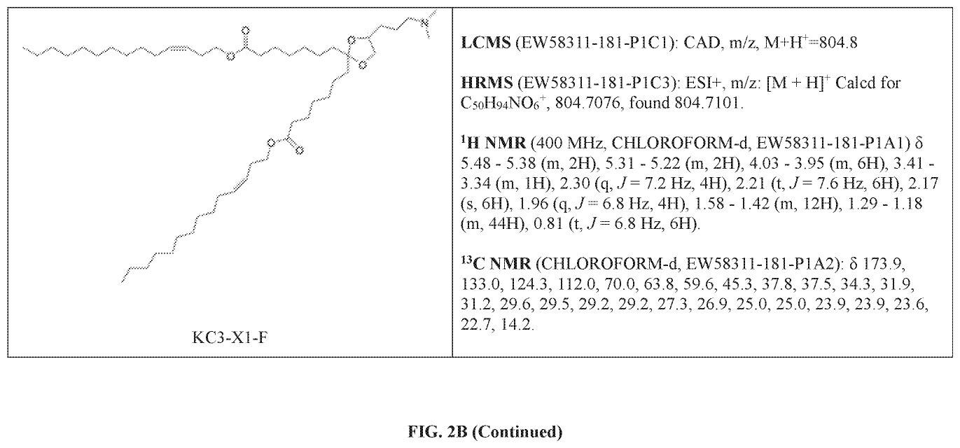

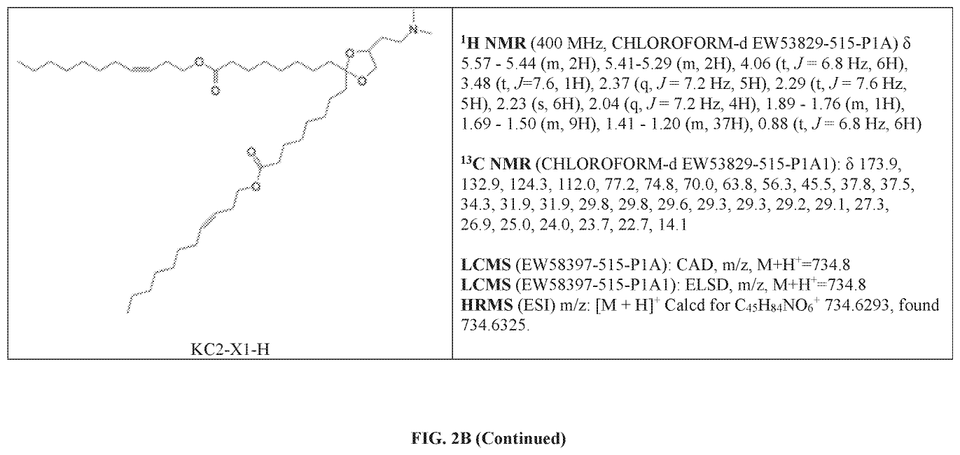

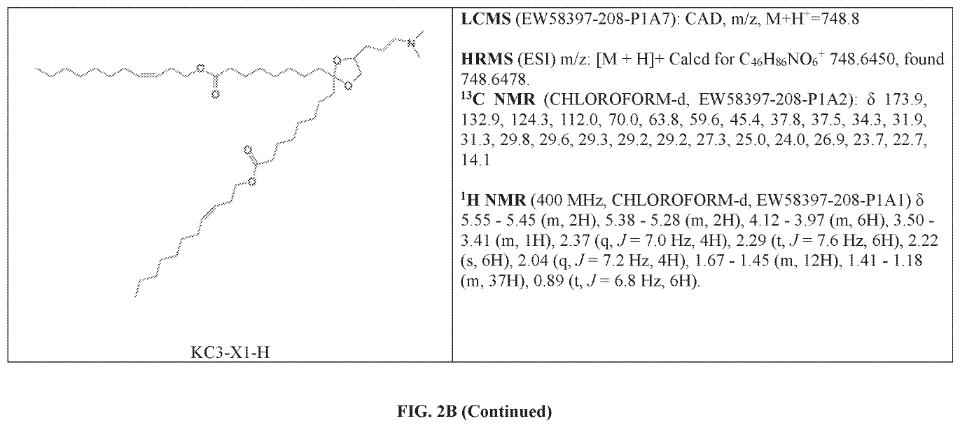

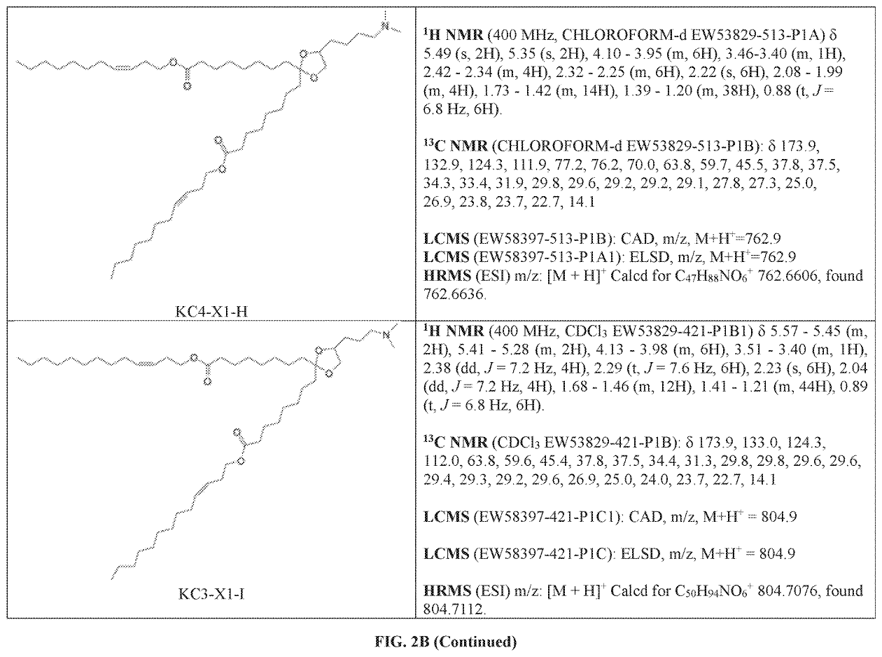

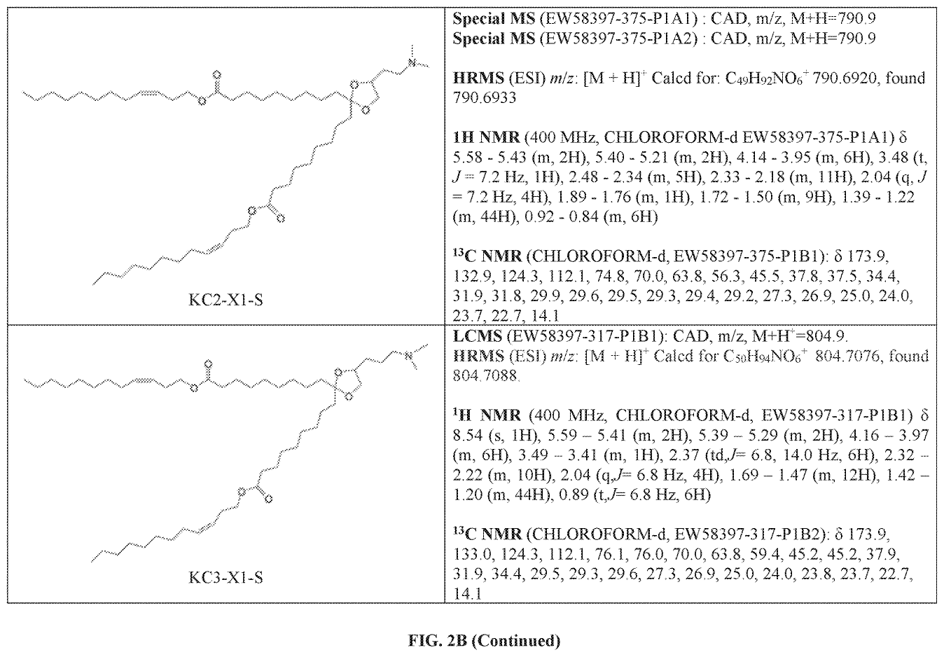

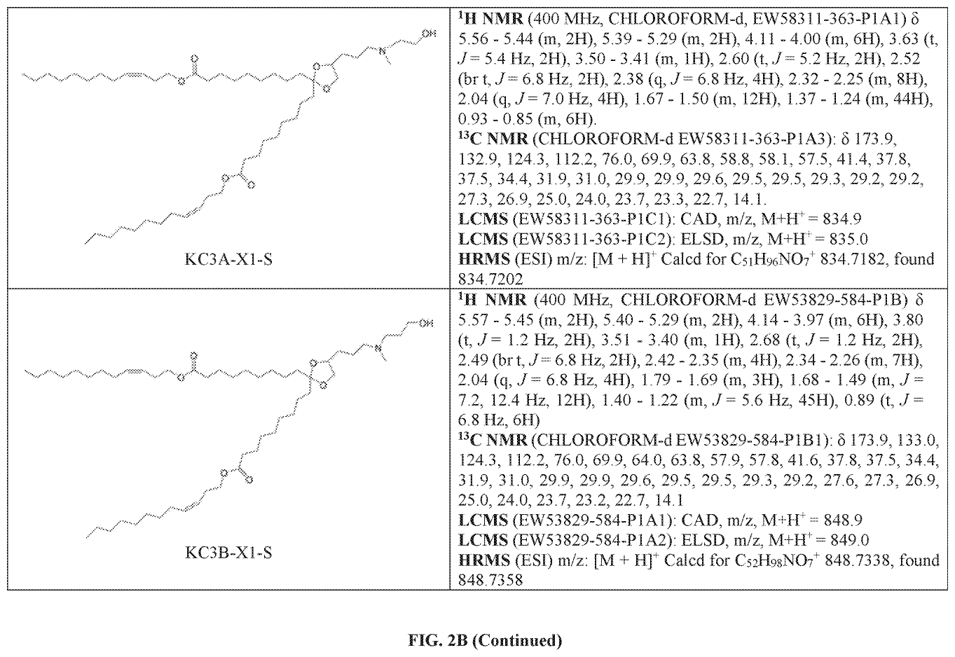

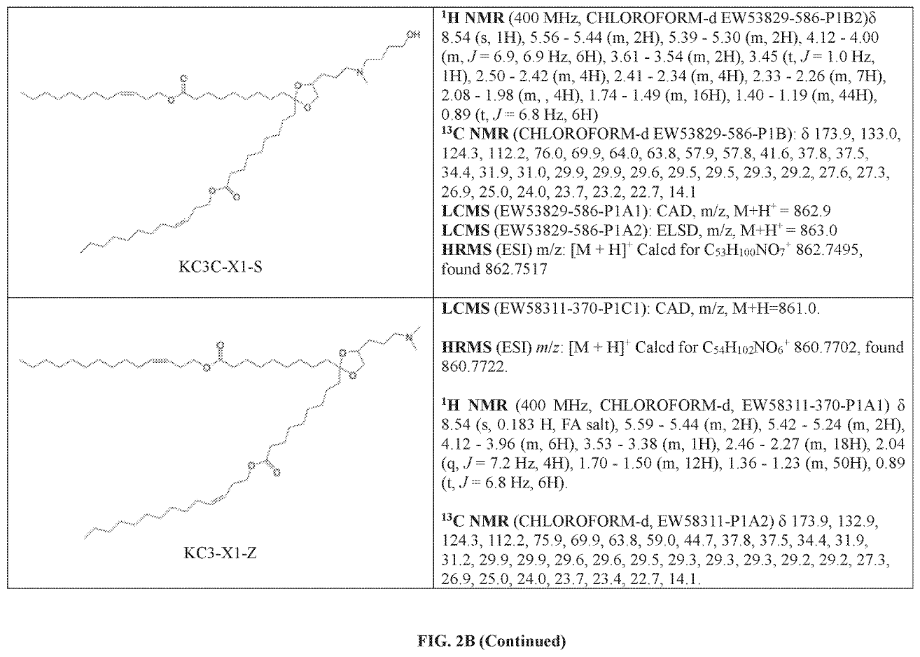

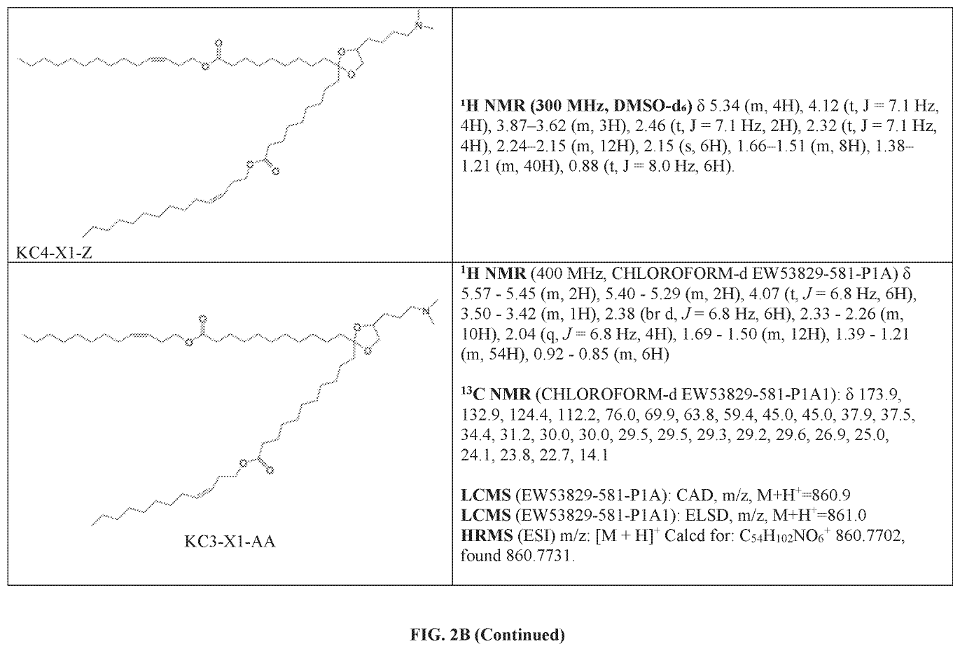

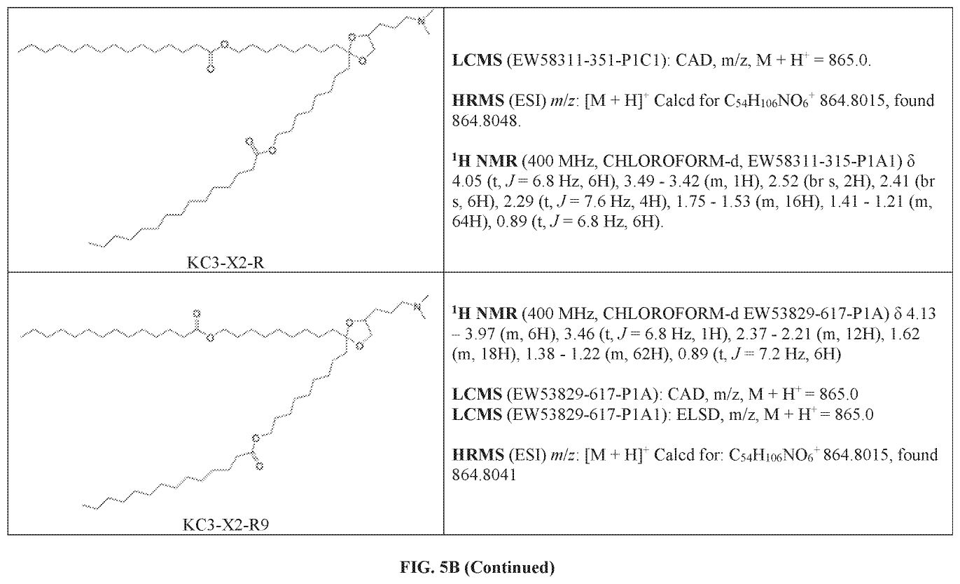

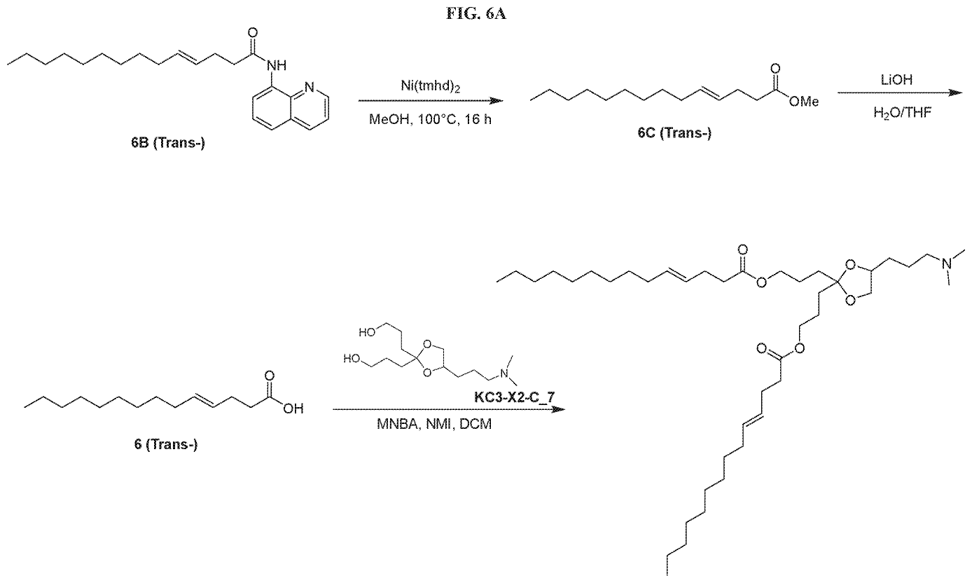

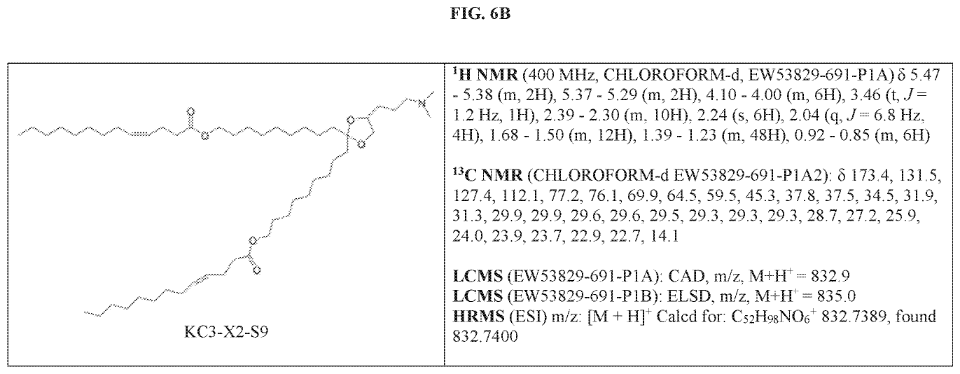

In some embodiments, an ionizable lipid compound consists of a dioxolane ring covalently bound to both (x) an alkylamine head group having a pKa of 6-7.5, and (y) a pair of identical geminal functionalized aliphatic chains at the 2-position of the dioxolane ring, each having 18-25 total carbon atoms and each consisting of (i) a 7-10 carbon linear aliphatic group covalently bound to the dioxolane ring and to (ii) an ester functional group, and (iii) a 10-20 carbon aliphatic chain covalently bound to the ester functional group. Non-limiting examples of ionizable cationic lipids include those listed in FIG. 1B, FIG. 2B, FIG. 3C, FIG. 4B, FIG. 5B, FIG. 6B, and FIG. 7B. In some embodiments, the dioxolane ring is a 1,3-dioxolane ring and each of the functionalized aliphatic chains comprises two or more branched C₅ hydrocarbon monomer units such as isopentane (IPN) or 2-methylbut-2-ene (2 MB). In some embodiments, an ionizable lipid comprises (a) other headgroups comprising an alkylamine having a pKa of 6-7.5, and (b) a pair of functionalized aliphatic chains comprising an ester functional group and two or more branched C₅ hydrocarbon monomer units such as isopentane (IPN) or 2-methylbut-2-ene (2 MB). For example, FIG. 8J is a table of additional exemplary ionizable cationic lipid compounds comprising a head group having an amine headgroup with a suitable pKa and two hydrophobic tails comprising one or more C₅-monomer units (2 MB) suitable to facilitate self-assembly into lipid nanoparticles. FIGS. 8A-8I are synthetic schemes for preparing additional exemplary ionizable cationic lipids.

KCn ionizable cationic lipid compounds of Formula (I) can be prepared using the chemical synthetic methods disclosed in the following Examples:

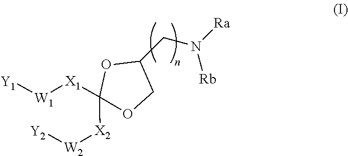

wherein X₁ and X₂ are each linear alkyl with (x) carbon atoms, W₁ and W₂ are each an ester, Y₁ and Y₂ are each alkyl or alkene with (y) carbon atoms, (n) is 2-4 and Rₐ and R_{b} are each C₁₋₄ alkyl optionally substituted with hydroxyl or alkoxy. In some embodiments, ICL compounds of Formula (I) having (x)=8-10 and/or (x+y)=21-24 are provided.

In compounds designated KCₙ-X1-, the W₁ and W₂ in Formula (I) are each *—C(O)O—** wherein * is a covalent bond to Y₁ or Y₂, respectively, and ** is a covalent bond to X₁ or X₂, respectively.

### Example 1. Synthesis of KCₙ-X1-Ionizable Cationic Lipids of Formula (I) Wherein X is —(CH₂)ₓ—, W is *—O—C(O)—** and Y is a Hydrocarbon with (y) Total Carbons

Scheme 1 is a general synthesis for compounds of Formula (I) wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear or branched hydrocarbon chains with (y) total carbons, (n) is 2, 3 or 4 and Ra and Rb are each C₁₋₄ alkyl optionally substituted with hydroxyl (i.e., “KCₙ—X1” ionizable cationic lipids). In some embodiments, X₁ and X₂ are the same, Y₁ and Y₂ are the same, and Rₐ and Rb are each methyl. In some embodiments, Ra is methyl and Rb is —(CH₂)₂—OH.

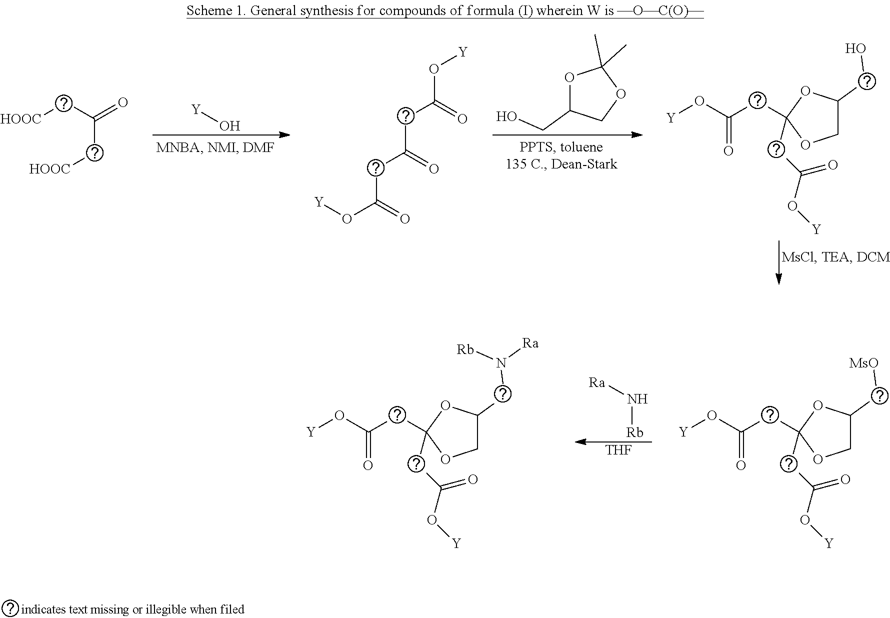

A general synthesis for compounds of formula (I) shown in Scheme 1 involves i) esterification of the ketone dicarboxylic acid, ii) acid catalyzed trans ketal formation, iii) Mesylation of the primary alcohol, and finally iv) displacement of the mesyl group using dialkylamine resulting in a tertiary amine. A general synthesis of ketone dicarboxylic acid starting material in Scheme 1 is presented below in Scheme 2.

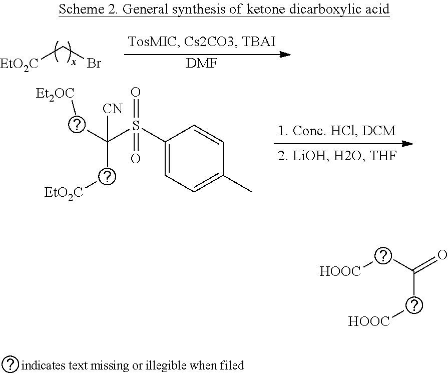

A general synthesis of ketone dicarboxylic acid starting material shown in Scheme 2 involves i) treatment of suitable bromo alkyl carboxylate with toluenesulfonylmethyl isocyanide in the presence of a base, ii) conversion of the TosMIC adduct to ketone in the presence of HCl, and finally iv) hydrolysis of the ethyl carboxylate to ketone dicarboxylic acid. Compounds with the prefix “AKG-” and without the prefix are used interchangeably.

### Example 1A. KCₙ—X1 Compounds of Formula (I) Comprising Linear Alkanes at Y₁ and Y₂

The following relates to compounds of Formula (I) wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkane hydrocarbon chains with (y) total carbons.

FIG. 1A is a chemical synthesis of compound of Formula (I) designated KC3-X1-K, wherein W₁ and W₂ are each *—O—C(O)—**, * is a covalent bond to Y₁ or Y₂ and ** is a covalent bond to X₁ or X₂ (respectively), X₁ or X₂ are each —(CH₂)₈—, and Y₁ or Y₂ are each —(CH₂)₁₂—.

To a solution of KC3-X1-L_int.5 (1.5 g, 4.38 mmol, 1 eq) in DCM (20 mL) was added DMAP (1.34 g, 10.95 mmol, 2.5 eq) and EDCI (3.36 g, 17.52 mmol, 4 eq), compound 1 (1.63 g, 8.76 mmol, 2 eq) and DIEA (5.66 g, 43.80 mmol, 7.63 mL, 10 eq). The mixture was stirred at 40° C. for 12 hr. The reaction mixture was concentrated under reduced pressure to remove DCM. The residue was purified by column chromatography (SiO₂, Petroleum ether/Ethyl acetate=1/0 to 98/2) to give compound 2 (1.2 g, 1.77 mmol, 40.3% yield) as a white solid.

¹H NMR (400 MHz, CHLOROFORM-d, EW58397-242-P1A1) δ 4.05 (t, J=6.8 Hz, 4H), 2.38 (t, J=7.4 Hz, 4H), 2.29 (t, J=7.5 Hz, 4H), 1.66-1.54 (m, 12H), 1.40-1.17 (m, 52H), 0.88 (t, J=6.8 Hz, 6H).

To a solution of compound 2 (1 g, 1.47 mmol, 1 eq) in Tol. (15 mL) was added PPTS (185.02 mg, 736.26 μmol, 0.5 eq) and compound 3 (471.83 mg, 2.95 mmol, 2 eq). The mixture was heated to reflux and remove water by Dean-Stark trap for 24 hrs. The reaction mixture was poured into saturated NaHCO₃ aqueous solution (40 mL) and extracted with EA (30 mL*3). The combined organic layers were washed with brine (20 mL*3), dried over Na₂SO₄, filtered and the filtrate concentrated in vacuum. The residue was purified by column chromatography (SiO₂, Petroleum ether/Ethyl acetate=1/0 to 91/9) to give compound 4 (812 mg, 1.04 mmol, 70.6% yield) as a colorless oil.

¹H NMR (400 MHz, CHLOROFORM-d, EW58397-246-P1A1) δ 4.13-4.03 (m, 6H), 3.74-3.62 (m, 2H), 3.51-3.43 (m, 1H), 2.29 (t, J=7.6 Hz, 4H), 1.72-1.53 (m, 17H), 1.37-1.23 (m, 56H), 0.89 (t, J=6.8 Hz, 6H).

To a solution of compound 4 (812 mg, 1.04 mmol, 1 eq) and TEA (525.87 mg, 5.20 mmol, 723.34 μL, 5 eq) in DCM (10 mL). Then added MsCl (357.19 mg, 3.12 mmol, 241.34 μL, 3 eq) at 0° C. The mixture was stirred at 25° C. for 2 hr. The reaction mixture was quenched by addition ice water (20 mL) and then extracted with DCM 30 mL (10 mL*3). The combined organic layers were washed with brine mL (20 mL*3), dried over Na₂SO₄, filtered and the filtrate concentrated under reduced pressure to give a residue. The residue was purified by column chromatography (SiO₂, Petroleum ether/Ethyl acetate=1/0 to 94/6) to give compound 5 (826 mg, 961.22 μmol, 92.5% yield) as a yellow oil. ¹H NMR (400 MHz, CHLOROFORM-d, EW58397-250-P1A1) δ 4.35-4.21 (m, 2H), 4.12-4.03 (m, 6H), 3.52-3.43 (m, 1H), 3.02 (s, 3H), 2.29 (t, J=7.6 Hz, 4H), 2.00-1.77 (m, 2H), 1.70-1.52 (m, 15H), 1.41-1.22 (m, 54H), 0.89 (t, J=6.8 Hz, 6H).

To a solution of compound 5 (700 mg, 814.59 μmol, 1 eq) in Me₂NH/THF (2 M, 10 μL, 1 eq). The mixture was stirred at 55° C. for 16 hr. The reaction mixture was concentrated under reduced pressure to remove THF. The residue was purified by column chromatography (SiO₂, [Petroleum ether/DCM,(0.1% TEA)=3/1]/Ethyl acetate=1/0 to 94/6) to give KC3-X1-K (488.1 mg, 587.67 μmol, 72.1% yield, 97.3% purity) as a yellow oil. ¹H NMR (400 MHz, CHLOROFORM-d, EW58397-252-P1A2) δ 4.14-3.97 (m, 6H), 3.52-3.39 (m, 1H), 2.29 (t, J=7.6 Hz, 6H), 2.23 (s, 6H), 1.67-1.46 (m, 16H), 1.37-1.24 (m, 56H), 0.89 (t, J=6.8 Hz, 6H). LCMS (EW58397-252-P1A1): CAD, m/z, M+H⁺=808.9. HRMS (ESI) m/z: [M+H]⁺ Calcd for C₅₀H₉₈NO₆⁺ 808.7389, found 808.7421.

FIG. 1B is a list of other exemplary compounds of Formula (I), wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkane hydrocarbon chains with (y) total carbons.

### Example 1B. KCₙ—X1 Compounds of Formula (I) Comprising Linear Alkenes at Y₁ and Y₂

The following relates to compounds of Formula (I) wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkene hydrocarbon chains with (y) total carbons.

FIG. 2A is a chemical synthesis of compound of Formula (I) designated KC3-X1-AA, wherein W₁ and W₂ are each *—O—C(O)—**, * is a covalent bond to Y₁ or Y₂ and ** is a covalent bond to X₁ or X₂ (respectively), X₁ or X₂ are each —(CH₂)₁₀—, and Y₁ or Y₂ are each —(CH₂)₂—CH═CH—(CH₂)₇CH₃.

To a solution of compound C23-1 (25 g, 85.25 mmol, 2.1 eq) in DMF (300 mL) was added compound 2 (7.93 g, 40.60 mmol, 1 eq), Cs₂CO₃ (39.68 g, 121.79 mmol, 3 eq) and TBAI (1.50 g, 4.06 mmol, 0.1 eq). The mixture was stirred at 50° C. for 1 hr. The reaction mixture was quenched by addition H₂O (200 mL) at 0° C., and then diluted with water 100 mL and extracted with EtOAc (100 mL*3). The combined organic layers were washed with brine (100 mL*2), dried over Na₂SO₄, filtered and concentrated under reduced pressure to give a residue. Compound C23-3 (26 g, crude) was obtained as a yellow oil. ¹H NMR (400 MHz, CHLOROFORM-d EW53829-516-P1A) δ 7.86 (m, 2H), 7.42 (t, J=7.6 Hz, 2H), 4.12 (q, J=7.2 Hz, 4H), 3.40 (t, J=6.8 Hz, 1H), 2.49 (s, 3H), 2.28 (t, J=7.6 Hz, 5H), 2.04-1.75 (m, 4H), 1.66-1.55 (m, 6H), 1.51-1.15 (m, 35H).

To a solution of compound C23-3 (26 g, 41.94 mmol, 1 eq) in DCM (210 mL) was added con.HCl (12 M, 70 mL, 20.03 eq). The mixture was stirred at 25° C. for 1 hr. The reaction mixture was quenched by addition H₂O 100 mL at 0° C. and extracted with DCM (100 mL*3). The combined organic layers were washed with brine (100 mL*2), dried over Na₂SO₄, filtered and concentrated under reduced pressure to give a residue. The residue was purified by column chromatography (SiO₂, PE:EA=50/1 to 10/1) to give compound C23-4 (7.4 g, 14.65 mmol, 34.92% yield, 90% purity) was obtained as a white solid. ¹H NMR (400 MHz, CHLOROFORM-d EW53829-518-P1A) δ 4.11 (q, J=7.2 Hz, 4H), 2.37 (t, J=7.6 Hz, 3H), 2.27 (t, J=7.6 Hz, 4H), 1.66-1.48 (m, 8H), 1.34-1.18 (m, 29H).

To a solution of compound C23-4 (7.4 g, 16.28 mmol, 1 eq) in THF (90 mL) and H2O (30 mL) was added LiOH·H₂O (4.10 g, 97.65 mmol, 6 eq). The mixture was stirred at 70° C. for 10 hr. The reaction was concentrated in vacuum, the mixture was adjusted to pH=5 with HCl aq. (2M). The crude product was triturated with H2O (100 mL) at 25° C. for 2 hr to give compound C23 acid (5 g, 6.27 mmol, 38.54% yield, 50% purity) was obtained as a white solid. ¹HNMR (400 MHz, DMSO-d6 EW53829-520-P1A1) δ 2.36 (br t, J=6.4 Hz, 4H), 2.17 (br t, J=6.8 Hz, 4H), 1.54-1.35 (m, 9H), 1.29-1.14 (m, 25H).

To a solution of compound C23 acid (1.7 g, 4.27 mmol, 1 eq) and compound 1 (2.36 g, 12.80 mmol, 3 eq) in DMF (20 mL) was added MNBA (4.41 g, 12.80 mmol, 3 eq) and NMI (3.50 g, 42.65 mmol, 3.40 mL, 10 eq). The mixture was stirred at 65° C. for 10 hr. The reaction mixture was concentrated under reduced pressure to remove solvent. The residue was purified by column chromatography (SiO₂, PE:EA=20/1 to 10/1) to give compound 2 (3.5 g, 3.59 mmol, 84% yield, 75% purity) as a white solid. ¹H NMR (400 MHz, CHLOROFORM-d EW53829-566-P1A) δ 5.50 (m, 4H), 5.43-5.29 (m, 4H), 4.38 (t, J=7.2 Hz, 4H), 4.06 (t, J=6.8 Hz, 4H), 2.51 (d, J=7.2 Hz, 4H), 2.43 (s, 6H), 2.38 (t, J=7.6 Hz, 8H), 2.28 (t, J=7.6 Hz, 4H), 2.04 (q, J=7.2 Hz, 8H), 1.59 (td, J=7.6, 14.8 Hz, 9H), 1.42-1.16 (m, 74H), 0.93-0.82 (m, 12H).

A mixture of compound 2 (3.5 g, 3.59 mmol, 1 eq), compound 3 and PPTS (451.09 mg, 1.80 mmol, 0.5 eq) in Tol. (40 mL) was heated to reflux and remove water by Dean-Stark trap. The mixture was stirred at 135° C. for 12 hr. The reaction mixture was quenched by addition NaHCO₃20 mL at 0° C., then diluted with water 30 mL and extracted with EtOAc (30 mL*3).

The combined organic layers were washed with H₂O 100 mL (50 mL*2), dried over Na₂SO₄, filtered and combined organic concentrated under reduced pressure to give a residue. The residue was purified by column chromatography (SiO₂, PE:EA=10/1 to 5/1) to give compound 4 (1.3 g, 1.40 mmol, 39% yield, 90% purity) was obtained as a yellow oil. ¹H NMR (400 MHz, CHLOROFORM-d EW53829-571-P1A) δ 5.57-5.45 (m, 2H), 5.41-5.28 (m, 2H), 4.07 (t, J=6.8 Hz, 6H), 3.76-3.63 (m, 2H), 3.52-3.43 (m, 1H), 2.38 (d, J=6.8 Hz, 4H), 2.29 (t, J=7.6 Hz, 5H), 2.04 (q, J=6.8 Hz, 4H), 1.74-1.51 (m, 15H), 1.41-1.20 (m, 53H), 0.93-0.85 (m, 6H).

To a solution of compound 4 (1.3 g, 1.56 mmol, 1 eq) in DCM (20 mL) was added TEA (789.30 mg, 7.80 mmol, 1.09 mL, 5 eq) and MsCl (680 mg, 5.94 mmol, 459.46 μL, 3.81 eq) at 0° C., the mixture was stirred at 25° C. for 2 hr. The reaction mixture was quenched by addition ice water 40 mL at 0° C. and extracted with DCM (30 mL*2). The combined organic layers were washed with brine (30 mL*2), dried over Na₂SO₄, filtered and the filtrate was concentrated under reduced pressure to give a residue. The residue was purified by column chromatography (SiO₂, PE (0.1% TEA)/EA=10/1 to 5/1) to give compound 5 (1.3 g, 1.43 mmol, 91% yield) as a yellow oil. ¹H NMR (400 MHz, CHLOROFORM-d EW53829-574-P1A) δ 5.56-5.45 (m, 2H), 5.36 (m, 2H), 4.29 (m, 2H), 4.07 (t, J=7.2 Hz, 6H), 3.50-3.44 (m, 1H), 3.02 (d, J=0.8 Hz, 3H), 2.43-2.35 (m, 4H), 2.29 (t, J=7.6 Hz, 4H), 2.04 (m, 4H), 1.97-1.79 (m, 2H), 1.74-1.52 (m, 13H), 1.27 (br s, 52H), 0.89 (t, J=6.4 Hz, 6H).

A solution of compound 5 (1.2 g, 1.32 mmol, 1 eq) in Me₂NH/THF (2 M, 22.63 mL) was stirred at 50° C. for 10 hr. The reaction mixture was concentrated under reduced pressure to remove solvent. The residue was purified by column chromatography (SiO₂, PE(0.1% TEA): EA=20/1 to 10/1) to give KC3-X1-AA (534.6 mg, 590.29 μmol, 63% yield, 95% purity) as a yellow oil. ¹H NMR (400 MHz, CHLOROFORM-d EW53829-581-P1A) δ 5.57-5.45 (m, 2H), 5.40-5.29 (m, 2H), 4.07 (t, J=6.8 Hz, 6H), 3.50-3.42 (m, 1H), 2.38 (br d, J=6.8 Hz, 6H), 2.33-2.26 (m, 10H), 2.04 (q, J=6.8 Hz, 4H), 1.69-1.50 (m, 12H), 1.39-1.21 (m, 54H), 0.92-0.85 (m, 6H)¹³C NMR (CHLOROFORM-d EW53829-581-P1A1): δ 173.9, 132.9, 124.4, 112.2, 76.0, 69.9, 63.8, 59.4, 45.0, 45.0, 37.9, 37.5, 34.4, 31.2, 30.0, 30.0, 29.5, 29.5, 29.3, 29.2, 29.6, 26.9, 25.0, 24.1, 23.8, 22.7, 14.1 LCMS (EW53829-581-P1A): CAD, m/z, M+H⁺=860.9 LCMS (EW53829-581-P1A1): ELSD, m/z, M+H⁺=861.0 HRMS (ESI) m/z: [M+H]⁺ Calcd for: C₅₄H₁₀₂NO₆⁺ 860.7702, found 860.7731.

FIG. 2B is a list of other exemplary compounds of Formula (I), wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkene hydrocarbon chains with (y) total carbons.

### Example 1C. KCₙ—X1 Compounds of Formula (I) Comprising Branched Alkanes at Y₁ and Y₂

The following relates to compounds of Formula (I) wherein W₁ and W₂ are each *—O—C(O)—** * is a covalent bond to Y₁ or Y₂ and ** is a covalent bond to X₁ or X₂ (respectively), X₁ or X₂ are each —(CH₂)ₓ—, and Y₁ or Y₂ are each identical branched alkane hydrocarbon chains with (y) total carbons.

FIG. 3A is a chemical synthesis of compound of Formula (I) designated KC3-X1-PH5 wherein W₁ and W₂ are each *—O—C(O)—**, * is a covalent bond to Y₁ or Y₂ and ** is a covalent bond to X₁ or X₂ (respectively), X₁ or X₂ are each —(CH₂)₇—, and Y₁ or Y₂ are each −(2 MB)₃ wherein 2 MB is a head-to-tail 2-methylbut-2-ene (2 MB) monomer unit.

To a solution of compound 1 (3 g, 9.54 mmol, 1 eq) and KC3-X1-PH1_2 (4.36 g, 19.08 mmol, 2 eq) in DCM (30 mL) was added EDCI (7.32 g, 38.17 mmol, 4 eq) and DMAP (2.91 g, 23.85 mmol, 2.5 eq) and DIEA (12.33 g, 95.42 mmol, 16.62 mL, 10 eq). The mixture was stirred at 40° C. for 12 hr. Upon completion, the mixture was concentrated in vacuo to give a residue. The residue was purified by flash silica gel chromatography (ISCO®; 80 g SepaFlash® Silica Flash Column, Eluent of 0~4% Ethyl acetate/Petroleum ether gradient @100 mL/min) to give compound 2 (6.8 g, 9.24 mmol, 96.8% yield, 99.9% purity) as a yellow solid. ¹H NMR (400 MHz, CHLOROFORM-d) δ 4.08-3.97 (m, 4H), 2.30 (t, J=7.2 Hz, 4H), 2.21 (t, J=7.6 Hz, 4H), 1.61-1.42 (m, 15H), 1.39-1.27 (m, 5H), 1.25-0.96 (m, 34H), 0.84-0.75 (m, 24H).

To a mixture of compound 2 (2 g, 2.72 mmol, 1 eq) and compound 3 (1.09 g, 6.80 mmol, 2.5 eq) in Tol. (20 mL) was added PPTS (341.81 mg, 1.36 mmol, 0.5 eq), and the mixture was heated to reflux and remove water by Dean-Stark trap for 12 hr. The reaction solution was added into saturated NaHCO₃ (30 mL) solution, and the aqueous phase was extracted three times with DCM (20 mL*3). The combined organic layers were washed with brine (20 mL), dried over Na₂SO₄, filtered and concentrated in vacuo. The residue was purified by flash silica gel chromatography (ISCO®; 40 g SepaFlash® Silica Flash Column, Eluent of 0~20% Ethyl acetate/Petroleum ether gradient @60 mL/min) to give compound 4 (1.8 g, 2.15 mmol, 79.1% yield) as a yellow oil. 1H NMR (400 MHz, CHLOROFORM-d, EW58311-220-P1A1) δ 4.18-4.07 (m, 6H), 3.72 (qd, J=5.2, 10.2 Hz, 2H), 3.54-3.45 (m, 1H), 2.32 (t, J=7.6 Hz, 4H), 1.72-1.52 (m, 18H), 1.50-1.22 (m, 34H), 1.21-1.04 (m, 10H), 1.01-0.78 (m, 24H).

To a solution of compound 4 (1.8 g, 2.15 mmol, 1 eq) in DCM (20 mL) was added TEA (1.09 g, 10.75 mmol, 1.50 mL, 5 eq) and MsCl (410 mg, 3.58 mmol, 277.03 μL, 1.67 eq). The mixture was stirred at 0° C. for 1 hr. The residue was poured into ice water (20 mL) and stirred for 2 min. The aqueous phase was extracted with DCM (20 mL*3). The combined organic phases were washed with brine (10 mL*3), dried with anhydrous Na₂SO₄, filtered and concentrated in vacuum. The residue was purified by flash silica gel chromatography (ISCO®; 40 g SepaFlash® Silica Flash Column, Eluent of 0~15% Ethyl acetate/Petroleum ether gradient @60 mL/min) to give compound 5 (1.6 g, 1.75 mmol, 81.3% yield) was as a yellow oil. ¹H NMR (400 MHz, CHLOROFORM-d, EW58311-223-P1A1) δ 4.35-4.25 (m, 2H), 4.15-4.04 (m, 6H), 3.52-3.45 (m, 1H), 3.04 (s, 3H), 2.30 (t, J=7.6 Hz, 4H), 2.00-1.79 (m, 2H), 1.71-1.52 (m, 17H), 1.50-1.23 (m, 34H), 1.18-1.04 (m, 10H), 0.94-0.85 (m, 24H).

FIG. 3B is a chemical synthesis of compound of Formula (I) designated KC3-X1-PH12 wherein W₁ and W₂ are each *—O—C(O)—**, * is a covalent bond to Y₁ or Y₂ and ** is a covalent bond to X₁ or X₂ (respectively), X₁ or X₂ are each —(CH₂)₁₀—, and Y₁ or Y₂ are each —(CH₂)-(2 MB)₂ wherein 2 MB is a head-to-tail 2-methylbut-2-ene (2 MB) monomer unit.

To a solution of KC3-X1-AA-C₂₃ acid (800 mg, 2.01 mmol, 1 eq) and KC3-X1-PH7_4 (1.12 g, 6.02 mmol, 3 eq) in DMF (10 mL) was added NMI (1.65 g, 20.07 mmol, 1.60 mL, 10 eq) and MNBA (2.07 g, 6.02 mmol, 3 eq). The mixture was stirred at 70° C. for 12 hr. The reaction mixture was quenched by addition aq. NaHCO₃ (sat. 30 mL) at 25° C., and extracted with EtOAc (10 mL*3). The combined organic layers were washed with brine (10 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0~3% Ethylacetate/Petroleum ether gradient @60 mL/min) to give compound 1 (1.5 g, 1.43 mmol, 71.2% yield, 70.1% purity) as a white solid. ¹H NMR (400 MHz, CHLOROFORM-d, EW58311-330-P1A1) δ 4.00 (t, J=6.8 Hz, 4H), 2.31 (t, J=7.4 Hz, 4H), 2.22 (t, J=7.6 Hz, 4H), 1.58-1.42 (m, 16H), 1.32-1.19 (m, 36H), 1.09-1.00 (m, 8H), 0.81-0.78 (m, 18H).

A mixture of compound 1 (1.5 g, 2.04 mmol, 1 eq), PPTS (256.36 mg, 1.02 mmol, 0.5 eq) and compound 2 (588.36 mg, 3.67 mmol, 1.8 eq) in Tol. (20 mL) was heated to reflux and remove water by Dean-Stark trap. The mixture was stirred at 135° C. for 12 hr. The reaction mixture was quenched by addition aq.NaHCO₃ (sat. 60 mL) at 25° C., and extracted with EtOAc (30 mL*3). The combined organic layers were washed with brine (20 mL*3), dried over Na₂SO₄, filtered and concentrated under reduced pressure to give a residue. The residue was purified by flash silica gel chromatography (ISCO®; 40 g SepaFlash® Silica Flash Column, Eluent of 0~10% Ethyl acetate/Petroleum ether gradient @60 mL/min) to give compound 3 (1 g, 1.19 mmol, 58.5% yield) as a yellow oil. 1H NMR (400 MHz, CHLOROFORM-d, EW58311-334-P1A1) δ 4.13-4.05 (m, 6H), 3.75-3.66 (m, 2H), 3.52-3.45 (m, 1H), 2.31 (t, J=7.6 Hz, 4H), 1.74-1.48 (m, 20H), 1.41-1.26 (m, 41H), 1.18-1.08 (m, 8H), 0.90-0.85 (m, 18H).

To a solution of compound 3 (1 g, 1.19 mmol, 1 eq) in DCM (10 mL) was added TEA (604.23 mg, 5.97 mmol, 831.13 μL, 5 eq) and MsCl (630 mg, 5.50 mmol, 425.68 μL, 4.61 eq). The mixture was stirred at 0° C. for 1 hr. The reaction mixture was quenched by addition ice-water (30 mL) at 0° C., and extracted with EtOAc (10 mL*3). The combined organic layers were washed with brine (10 mL*3), dried over Na₂SO₄, filtered and concentrated under reduced pressure to give a residue. The residue was purified by flash silica gel chromatography (ISCO®; 12 g SepaFlash® Silica Flash Column, Eluent of 0~15% Ethyl acetate/Petroleum ether gradient @40 mL/min) to give compound 4 (1 g, 1.09 mmol, 91.5% yield) as a yellow oil. ¹H NMR (400 MHz, CHLOROFORM-d, EW58311-335-P1A1) δ 4.36-4.24 (m, 2H), 4.12-4.03 (m, 6H), 3.54-3.44 (m, 1H), 3.03 (s, 3H), 2.31 (t, J=7.6 Hz, 4H), 1.99-1.79 (m, 2H), 1.69-1.52 (m, 20H), 1.40-1.27 (m, 38H), 1.19-1.06 (m, 8H), 0.94-0.83 (m, 18H).

A solution of compound 4 (900 mg, 983.14 μmol, 1 eq) in Me₂NH/THF (2 M, 20 mL, 40.69 eq) was stirred at 55° C. for 12 hr. The mixture was concentrated in vacuo to give a residue. The residue was purified by flash silica gel chromatography (ISCO®; 20 g SepaFlash® Silica Flash Column, Eluent of 0~8% Ethyl acetate/(Petroleum ether/Dichloromethane=3/1, 0.1% TEA as eluant) @40 mL/min) and followed by reversed-phase HPLC (column: PHS XP Phenyl-Hexyl 150*25 mm*7 um; mobile phase: [H₂O (0.225% FA)-EtOH]; gradient: 65%-95% B over 15.0 min). KC3-X1-PH12 (0.094 eq. FA salt, 435.5 mg, 495.75 μmol, 53.6% yield, 98.4% purity) was obtained as a colorless oil. LCMS (EW58311-340-P1C1): CAD, m/z, M+H=865.0. HRMS (ESI) m/z: [M+H]⁺ Calcd for C₅₄H₁₀₆NO₆⁺ 864.8015, found 864.8044. ¹H NMR (400 MHz, CHLOROFORM-d, EW58311-340-P1A1) δ 8.55 (s, 0.094H, FA salt), 4.15-3.98 (m, 6H), 3.52-3.40 (m, 1H), 2.36 (br t, J=7.2 Hz, 2H), 2.32-2.26 (m, 10H), 1.67-1.47 (m, 18H), 1.41-1.19 (m, 39H), 1.16-1.00 (m, 8H), 0.93-0.76 (m, 18H).

FIG. 3C is a list of other exemplary compounds of Formula (I), wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical branched alkane hydrocarbon chains with (y) total carbons (including one or more 2 MB monomer units).

### Example 1D. KCₙ—X1 Compounds of Formula (I) Comprising Branched Alkenes at Y₁ and Y₂

The following relates to compounds of Formula (I) wherein W₁ and W₂ are each *—O—C(O)—** * is a covalent bond to Y₁ or Y₂ and ** is a covalent bond to X₁ or X₂ (respectively), X₁ or X₂ are each —(CH₂)ₓ—, and Y₁ or Y₂ are each identical branched alkene hydrocarbon chains with (y) total carbons.

FIG. 4A is a chemical synthesis of compound of Formula (I) designated KC3-X1-PH1B wherein W₁ and W₂ are each *—O—C(O)—**, * is a covalent bond to Y₁ or Y₂ and ** is a covalent bond to X₁ or X₂ (respectively), X₁ or X₂ are each —(CH₂)₁₀—, and Y₁ or Y₂ are each —(IPN)₃ wherein IPN is a head-to-tail isopentane (IPN) monomer unit.

To a solution of X1-E_int.9 (1 g, 2.41 mmol, 1 eq) in DMF (20 mL) was added MNBA (3.73 g, 10.83 mmol, 4.5 eq) and NMI (1.28 g, 15.64 mmol, 1.25 mL, 6.5 eq), then added the compound 1 (1.61 g, 7.22 mmol, 3 eq). The mixture was stirred at 40° C. for 16 hr. The residue was poured into water (30 mL) and stirred for 2 min. The aqueous phase was extracted with ethyl acetate (20 mL*3). The combined organic phase was washed with brine (10 mL*3), dried with anhydrous Na₂SO₄, filtered and concentrated in vacuum. The residue was purified by column chromatography (SiO2, [Petroleum ether/DCM, (0.1% TEA)=3/1]/Ethyl acetate=1/0 to 90/10) to give KC3-X1-PH1-B (369.3 mg, 425.86 μmol, 17.7% yield, 95.1% purity) as a yellow oil. LCMS (EW58397-65-P1A1): CAD, m/z, M+H⁺=824.8. HRMS (ESI) m/z: [M+H]⁺ Calcd for C₅₂H₉₀NO₆⁺ 824.6763, found 824.6788. ¹H NMR (400 MHz, CHLOROFORM-d) δ 5.35 (br t, J=7.2 Hz, 2H), 5.16-5.04 (m, 4H), 4.59 (d, J=7.2 Hz, 4H), 4.10-4.01 (m, 2H), 3.49-3.42 (m, 1H), 2.30 (t, J=7.6 Hz, 6H), 2.23 (s, 6H), 2.17-2.02 (m, 13H), 2.02-1.95 (m, 4H), 1.70 (d, J=9.6 Hz, 13H), 1.66-1.43 (m, 21H), 1.31 (br d, J=2.0 Hz, 13H). ³C NMR (CHLOROFORM-d): δ 173.9, 142.1, 135.5, 131.3, 124.3, 123.7, 118.5, 112.0, 77.2, 76.2, 70.0, 61.2, 59.6, 45.4, 39.7, 39.6, 37.8, 37.5, 34.4, 31.3, 29.6, 29.2, 29.2, 26.7, 26.2, 25.7, 25.0, 25.0, 24.0, 23.9, 23.6, 17.7, 16.5, 16.0.

To a solution of compound 1 (3 g, 9.54 mmol, 1 eq) and KC3-X1-PH1_2 (4.36 g, 19.08 mmol, 2 eq) in DCM (30 mL) was added EDCI (7.32 g, 38.17 mmol, 4 eq) and DMAP (2.91 g, 23.85 mmol, 2.5 eq) and DIEA (12.33 g, 95.42 mmol, 16.62 mL, 10 eq). The mixture was stirred at 40° C. for 12 hr. Upon completion, the mixture was concentrated in vacuo to give a residue. The residue was purified by flash silica gel chromatography (ISCO®; 80 g SepaFlash® Silica Flash Column, Eluent of 0~4% Ethyl acetate/Petroleum ether gradient @100 mL/min) to give compound 2 (6.8 g, 9.24 mmol, 96.8% yield, 99.9% purity) as a yellow solid. 1H NMR (400 MHz, CHLOROFORM-d) δ 4.08-3.97 (m, 4H), 2.30 (t, J=7.2 Hz, 4H), 2.21 (t, J=7.6 Hz, 4H), 1.61-1.42 (m, 15H), 1.39-1.27 (m, 5H), 1.25-0.96 (m, 34H), 0.84-0.75 (m, 24H).

FIG. 4B is a list of other exemplary compounds of Formula (I), wherein W₁ and W₂ are each *—O—C(O)—**, * is a covalent bond to Y₁ or Y₂ and ** is a covalent bond to X₁ or X₂ (respectively), X₁ or X₂ are each —(CH₂)ₓ—, and Y₁ or Y₂ are each identical branched alkene hydrocarbon chains with (y) total carbons (including one or more IPN monomer units).

### Example 1E. Non-KCₙ Compounds Comprising Branched Alkanes at Y₁ and Y₂

FIG. 8A is a chemical synthesis of a compound designated DODAP-X1-PH12.

To a solution of Core A (KC3-X1-PH7_4, FIG. 3B) (3.00 g, 16.1 mmol, 1.00 eq) and compound 1b (5.56 g, 24.2 mmol, 1.50 eq) in DCM (100 mL) was added EDCI (3.70 g, 19.3 mmol, 1.20 eq) and DMAP (1.57 g, 12.9 mmol, 0.80 eq). The mixture was stirred at 25° C. for 12 hrs. The reaction mixture was concentrated under reduced pressure to remove solvent. The residue was purified by column chromatography (SiO₂, PE/EtOAc=50/1 to 10/1) to give compound 1 (4.00 g, 9.33 mmol, 58.0% yield) as a colorless oil.

¹H NMR (400 MHz, CHLOROFORM-d, EW53829-915-P1A1) δ 4.07 (t, J=2.8 Hz, 2H), 2.36 (t, J=7.6 Hz, 2H), 2.30 (t, J=7.6 Hz, 2H), 1.74-1.45 (m, 8H), 1.40-1.25 (m, 18H), 1.18-1.06 (m, 4H), 0.87 (d, J=6.4 Hz, 6H), 0.86 (d, J=6.8 Hz, 3H).

To a solution of OA-DM2_4 (300 mg, 1.63 mmol, 1.00 eq) and compound 1 (1.50 g, 3.76 mmol, 2.30 eq) in DCM (10 mL) was added EDCI (1.25 g, 6.53 mmol, 4.00 eq) and DMAP (499 mg, 4.08 mmol, 2.50 eq) and DIEA (2.11 g, 16.3 mmol, 2.84 mL, 10.0 eq). The mixture was stirred at 40° C. for 12 hrs. The reaction mixture was concentrated under reduced pressure to remove solvent. The residue was purified by column chromatography (SiO₂, PE (0.1% TEA)/EtOAc=50/1 to 10/1) to give DODAP-X1-PH12 (510 mg, 539 μmol, 33.0% yield, 96.4% purity) as a colorless oil. LCMS (EW53829-920-P1A): CAD, m/z: [M+H]⁺=909.0. HRMS (ESI) m/z: [M+H]⁺ Calcd for: C₅₅H₁₀₆NO₈⁺ 908.7913, found 908.7930. ¹H NMR (400 MHz, CHLOROFORM-d, EW53829-920-P1A) δ 5.16-5.06 (m, 1H), 4.28-4.19 (m, 1H), 4.13-3.98 (m, 5H), 2.35-2.25 (m, 10H), 2.22 (s, 6H), 1.71-1.46 (m, 20H), 1.44-1.21 (m, 36H), 1.20-1.05 (m, 8H), 0.87 (d, J=6.8 Hz, 12H), 0.86 (d, J=6.4 Hz, 6H).

To a solution of KC3-X1-PH1_2 (FIG. 3A) (3.00 g, 13.1 mmol, 1.00 eq) and compound 1 (2.47 g, 13.1 mmol, 1.00 eq) in DCM (30 mL) was added EDCI (3.02 g, 15.8 mmol, 1.20 eq) and DMAP (1.28 g, 10.5 mmol, 0.80 eq). The mixture was stirred at 25° C. for 12 hrs. The reaction mixture was concentrated under reduced pressure to remove solvent. The residue was purified by column chromatography (SiO₂, PE/EtOAc=50/1 to 10/1) to give compound 2 (800 mg, 1.97 mmol, 15.0% yield) as a colorless oil. ¹H NMR (400 MHz, CHLOROFORM-d, EW53829-892-P1A) δ 4.20-4.04 (m, 2H)(overlap with EtOAc signal), 2.35 (t, J=7.6 Hz, 2H), 2.29 (t, J=7.6 Hz, 2H), 1.73-1.48 (m, 7H), 1.45-1.05 (m, 20H) (overlap with EtOAc signal), 0.95-0.80 (m, 12H).

To a solution of OA-DM2_4 (160 mg, 871 μmol, 1.00 eq) and compound 2 (764 mg, 1.92 mmol, 2.20 eq) in DCM (10 mL) was added EDCI (668 mg, 3.48 mmol, 4.00 eq) and DMAP (266 mg, 2.18 mmol, 2.50 eq), and DIEA (1.13 g, 8.71 mmol, 1.52 mL, 10.0 eq). The mixture was stirred at 40° C. for 12 hrs. The reaction mixture was concentrated under reduced pressure to remove solvent. The residue was purified by column chromatography (SiO₂, PE (0.1% TEA)/EtOAc=50/1 to 10/1) and purified by prep-HPLC (column: PHS XP Phenyl-Hexyl 150*25 mm*7 um; mobile phase: [H₂O (0.225% FA) -EtOH]; gradient: 65%-95% B over 15.0 min) to give DODAP-X1-PH5 (161.3 mg, 176 μmol, 20.0% yield, 99.7% purity) as a colorless oil. LCMS (EW53829-910-P1A2): CAD, m/z: [M+H]⁺=909.0. HRMS (ESI) m/z: [M+H]⁺ Calcd for: Chemical Formula: C₅₅H₁₀₆NO₈⁺ 908.7913, found 908.7952. ¹H NMR (400 MHz, CHLOROFORM-d, EW53829-910-P1B1) δ5.15-5.04 (m, 1H), 4.23 (dd, J=12.0, 3.6 Hz, 1H), 4.16-3.99 (m, 5H), 2.33-2.25 (m, 10H), 2.22 (s, 6H), 1.72-1.48 (m, 20H), 1.38-1.21 (m, 27H), 1.19-1.00 (m, 11H), 0.93-0.82 (m, 24H).

FIG. 8B is a chemical synthesis of a compound designated DODAP-X1-PH5.

To a solution of KC3-X1-PH1_2 (FIG. 3A) (3.00 g, 13.1 mmol, 1.00 eq) and compound 1 (2.47 g, 13.1 mmol, 1.00 eq) in DCM (30 mL) was added EDCI (3.02 g, 15.8 mmol, 1.20 eq) and DMAP (1.28 g, 10.5 mmol, 0.80 eq). The mixture was stirred at 25° C. for 12 hrs. The reaction mixture was concentrated under reduced pressure to remove solvent. The residue was purified by column chromatography (SiO₂, PE/EtOAc=50/1 to 10/1) to give compound 2 (800 mg, 1.97 mmol, 15.0% yield) as a colorless oil. ¹H NMR (400 MHz, CHLOROFORM-d, EW53829-892-P1A) δ 4.20-4.04 (m, 2H)(overlap with EtOAc signal), 2.35 (t, J=7.6 Hz, 2H), 2.29 (t, J=7.6 Hz, 2H), 1.73-1.48 (m, 7H), 1.45-1.05 (m, 20H) (overlap with EtOAc signal), 0.95-0.80 (m, 12H).

To a solution of OA-DM2_4 (160 mg, 871 μmol, 1.00 eq) and compound 2 (764 mg, 1.92 mmol, 2.20 eq) in DCM (10 mL) was added EDCI (668 mg, 3.48 mmol, 4.00 eq) and DMAP (266 mg, 2.18 mmol, 2.50 eq), and DIEA (1.13 g, 8.71 mmol, 1.52 mL, 10.0 eq). The mixture was stirred at 40° C. for 12 hrs. The reaction mixture was concentrated under reduced pressure to remove solvent. The residue was purified by column chromatography (SiO₂, PE (0.1% TEA)/EtOAc=50/1 to 10/1) and purified by prep-HPLC (column: PHS XP Phenyl-Hexyl 150*25 mm*7 um; mobile phase: [H₂O (0.225% FA)-EtOH]; gradient: 65%-95% B over 15.0 min) to give DODAP-X1-PH5 (161.3 mg, 176 μmol, 20.0% yield, 99.7% purity) as a colorless oil. LCMS (EW53829-910-P1A2): CAD, m/z: [M+H]⁺=909.0. HRMS (ESI) m/z: [M+H]⁺ Calcd for: Chemical Formula: C₅₅H₁₀₆NO₈⁺ 908.7913, found 908.7952. ¹H NMR (400 MHz, CHLOROFORM-d, EW53829-910-P1B1) δ5.15-5.04 (m, 1H), 4.23 (dd, J=12.0, 3.6 Hz, 1H), 4.16-3.99 (m, 5H), 2.33-2.25 (m, 10H), 2.22 (s, 6H), 1.72-1.48 (m, 20H), 1.38-1.21 (m, 27H), 1.19-1.00 (m, 11H), 0.93-0.82 (m, 24H)

FIG. 8C is a chemical synthesis of compounds designated MA-X1-PH12 and MAE-X1-PH12.

To a solution of compound 1 (2.78 g, 10.5 mmol, 1.30 eq) in DCM (30.0 mL) was added EDCI (1.85 g, 9.66 mmol, 1.20 eq), Core A (1.50 g, 8.05 mmol, 1.00 eq) and DMAP (492 mg, 4.03 mmol, 0.50 eq). The mixture was stirred at 25° C. for 12 hrs. The reaction mixture was concentrated under reduced pressure to remove solvent. The residue was purified by column chromatography (SiO₂, petroleum ether/ethyl acetate=1/0 to 97/3) to give compound 2 (2.88 g, 6.64 mmol, 82.5% yield) as a colorless oil. ¹H NMR (400 MHz, CHLOROFORM-d, EW58397-576-P1A1) δ4.06 (t, J=6.8 Hz, 2H), 3.41 (t, J=6.8 Hz, 2H), 2.29 (t, J=7.6 Hz, 2H), 1.85 (q, J=7.2 Hz, 2H), 1.66-1.56 (m, 4H), 1.52 (q, J=6.8 Hz, 1H), 1.47-1.21 (m, 19H), 1.19-1.03 (m, 4H), 0.87 (d, J=6.8 Hz, 6H), 0.85 (d, J=6.4 Hz, 3H).

To a solution of compound 2 (2.41 g, 5.57 mmol, 2.80 eq) in ACN (15.0 mL) was added K₂CO₃ (824 mg, 5.96 mmol, 3.00 eq), (4-methoxyphenyl)methanamine (300 mg, 2.19 mmol, 283 μL, 1.10 eq), NaI (59.6 mg, 398 μmol, 0.20 eq) and Cs₂CO₃ (648 mg, 1.99 mmol, 1.00 eq). The mixture was stirred at 80° C. for 10 hrs. The reaction mixture was concentrated under reduced pressure to remove solvent. The residue was purified by column chromatography (SiO₂, petroleum ether/ethyl acetate=1/0 to 94/6) to give compound 3 (1.30 g, 1.54 mmol, 77.6% yield) as a colorless oil.

¹H NMR (400 MHz, CHLOROFORM-d, EW58397-587-P1A1) δ7.23 (d, J=8.4 Hz, 2H), 6.84 (d, J=8.4 Hz, 2H), 4.07 (t, J=6.8 Hz, 4H), 3.81 (s, 3H), 3.48 (s, 2H), 2.37 (t, J=7.2 Hz, 4H), 2.29 (t, J=7.6 Hz, 4H), 1.66-1.57 (m, 8H), 1.53 (q, J=6.8 Hz, 2H), 1.48-1.39 (m, 6H), 1.37-1.22 (m, 36H), 1.18-1.05 (m, 8H), 0.87 (d, J=6.8 Hz, 12H), 0.86 (d, J=6.8 Hz, 6H).

To the colorless solution of compound 3 (1.30 g, 1.54 mmol, 1.00 eq) in EtOAc (20.0 mL) and AcOH (5.00 mL) was added Pd(OH)₂/C (217 mg, 309 μmol, 20.0% purity, 0.20 eq) under N₂. The suspension was degassed under vacuum and purged with H₂ 3 times. The reaction mixture stirred at 25° C. for 12 hrs under H₂ (50 psi). The reaction mixture was filtered and the filtrate was concentrated under reduced pressure. Sat. Na₂CO₃ (20 mL) was added to the reaction mixture to adjust the pH to 7 and then diluted with H₂O (30 mL) and extracted with EtOAc (20 mL*3). The combined organic layers were washed with brine (20 mL*3), dried over Na₂SO₄, filtered and the filtrate was concentrated under reduced pressure to give a residue. The residue was purified by column chromatography (SiO₂, petroleum ether/ethyl acetate=1/0 to 90/10) to give compound 4 (1.00 g, 1.20 mmol, 78.1% yield) as a colorless oil. ¹H NMR (400 MHz, CHLOROFORM-d, EW58397-596-P1A2) 64.06 (t, J=6.8 Hz, 4H), 2.76 (t, J=8.0 Hz, 4H), 2.29 (t, J=7.6 Hz, 4H), 1.73-1.56 (m, 12H), 1.52 (q, J=6.8 Hz, 2H), 1.42-1.21 (m, 38H), 1.17-1.03 (m, 8H), 0.87 (d, J=6.8 Hz, 12H), 0.85 (d, J=6.4 Hz, 6H).

To a solution of compound 4 (700 mg, 969 μmol, 1.00 eq) in DCM (14.0 mL) was added EDCI (223 mg, 1.16 mmol, 1.20 eq), compound 5 (325 mg, 1.94 mmol, 2.00 eq, HCl) and DMAP (94.7 mg, 775 μmol, 0.80 eq). The mixture was stirred at 25° C. for 1 hr. The reaction mixture was concentrated under reduced pressure to remove solvent. The reaction mixture was quenched by addition sat. NaHCO₃ (30 mL) at 25° C. and then diluted with H₂O (20 mL) and extracted with EtOAc (30 mL*3). The combined organic layers were washed with brine (20 mL*3), dried over Na₂SO₄, filtered and the filtrate was concentrated under reduced pressure to give a residue. The residue was purified by column chromatography (SiO₂, [petroleum ether/DCM (0.1% TEA)=3/1]/ethyl acetate=1/0 to 91/9) to give MAE-X1-PH12 (483.7 mg, 573 mol, 59.1% yield, 98.9% purity) as a yellow oil. LCMS (EW58397-609-P1A1): CAD, m/z: [M+H]⁺=836.0. HRMS (ESI) m/z: [M+H]⁺ Calcd for C₅₂H₁₀₃N2O+835.7862, found 835.7903. ¹H NMR (400 MHz, CHLOROFORM-d, EW58397-609-P1A2) δ 4.07 (t, J=6.8 Hz, 4H), 3.29 (t, J=7.6 Hz, 2H), 3.21 (t, J=7.6 Hz, 2H), 2.41-2.27 (m, 8H), 2.25 (s, 6H), 1.83 (q, J=6.8 Hz, 2H), 1.66-1.48 (m, 14H), 1.43-1.22 (m, 38H), 1.18-1.06 (m, 8H), 0.87 (d, J=6.8 Hz, 12H), 0.86 (d, J=6.4 Hz, 6H).

To a solution of compound 2 (2.00 g, 4.61 mmol, 1.00 eq) in ACN (30.0 mL) was added DIEA (1.13 g, 8.72 mmol, 1.52 mL, 1.89 eq) and MeNH₂/THF (2.00 M, 1.85 mL, 0.80 eq) and ACN (30.0 mL). The mixture was stirred at 75° C. for 12 hrs. The reaction mixture was quenched by addition water (30 mL) at 0° C. and extracted with DCM (10 mL*3). The combined organic layers were washed with brine (10 mL*3), dried over Na₂SO₄, filtered and concentrated under reduced pressure to give a residue. The residue was purified by column chromatography (SiO₂, [petroleum ether/DCM (0.1% TEA)=3/1]/ethyl acetate=1/0 to 91/9) to give MA-X1-PH12 (512.2 mg, 692 μmol, 15.0% yield, 99.4% purity) as a colorless oil. LCMS (EW58397-603-P1A1): CAD, m/z: [M+H]⁺=736.9. HRMS (ESI) m/z: [M+H]⁺ Calcd for C₄₇H9₄NO₄⁺ 736.7178, found 736.7196. ¹H NMR (400 MHz, CHLOROFORM-d, EW58397-603-P1A2) δ 4.07 (t, J=6.8 Hz, 4H), 2.37-2.24 (m, 8H), 2.21 (s, 3H), 1.73-1.48 (m, 12H), 1.47-1.24 (m, 40H), 1.18-1.03 (m, 8H), 0.87 (d, J=6.8 Hz, 12H), 0.86 (d, J=6.8 Hz, 6H).

FIG. 8D is a chemical synthesis of compounds designated DMA3E-X1-PH12 and DMA4E-X1-12.

To a solution of compound 1 (Compound 1, FIG. 7A) (895 mg, 1.22 mmol, 1.00 eq) in THF (10 mL) was added EtOH (168 mg, 3.65 mmol, 0.10 mL, 3.00 eq) and NaBH₄ (430 mg, 11.4 mmol, 9.34 eq). The mixture was stirred at 40° C. for 12 hrs under N₂ atmosphere. The reaction mixture was quenched by addition of NH₄Cl solution (50 mL) at 25° C. under N₂ atmosphere and extracted with EA, 30 mL*3. The combined organic layers were washed with brine (30 mL*3), dried over Na₂SO₄, filtered and concentrated under reduced pressure to give compound 2 (603 mg, 797 μmol, 65.5% yield, 97.4% purity) as a colorless oil. ¹H NMR (400 MHz, CHLOROFORM-d, EW58397-543-P1A2) δ 4.07 (t, J=6.4 Hz, 4H), 3.59 (s, 1H), 2.30 (t, J=7.6 Hz, 4H), 1.66-1.52 (m, 10H), 1.47-1.24 (m, 46H), 1.18-1.03 (m, 8H), 0.87 (d, J=6.4 Hz, 12H), 0.86 (d, J=6.8 Hz, 6H).

To a solution of compound 2 (503 mg, 682 μmol, 1.00 eq) inDCM (10 mL) was added EDCI (157 mg, 819 μmol, 1.20 eq), compound 3 (107 mg, 641 μmol, 0.94 eq, HCl) and DMAP (66.7 mg, 546 μmol, 0.80 eq). The mixture was stirred at 25° C. for 12 hrs. The reaction mixture was concentrated under reduced pressure to remove solvent. The residue was purified by column chromatography (SiO₂, [petroleum ether/DCM (0.1% TEA)=3/1]/ethyl acetate=1/0 to 91/9) to give DMA3E-X1-PH12 (426.3 mg, 501 μmol, 82.3% yield, 98.1% purity) as a colorless oil. LCMS (EW58397-550-P1A1): CAD, m/z, [M+H]⁺=851.0. HRMS (ESI) m/z: [M+H]⁺ Calcd for C₅₃H₁₀₄NO₆⁺ 850.7858, found 850.7875. ¹H NMR (400 MHz, CHLOROFORM-d, EW58397-550-P1A4) δ 4.87 (q, J=6.4 Hz, 1H), 4.06 (t, J=6.8 Hz, 4H), 2.39-2.25 (m, 8H), 2.23 (s, 6H), 1.79 (q, J=7.6 Hz, 2H), 1.68-1.46 (m, 14H), 1.43-1.20 (m, 42H), 1.18-1.01 (m, 8H), 0.87 (d, J=6.8 Hz, 12H), 0.85 (d, J=6.8 Hz, 6H).

To a solution of compound 2 (1.20 g, 1.63 mmol, 1.00 eq) in DCM (20 mL) was added EDCI (780 mg, 4.07 mmol, 2.50 eq), compound 4 (320 mg, 2.20 mmol, 1.35 eq) and DMAP (199 mg, 1.63 mmol, 1.00 eq). The mixture was stirred at 40° C. for 12 hrs. The reaction mixture was concentrated under reduced pressure to remove solvent. The residue was purified by column chromatography (SiO₂, [petroleum ether/DCM (0.1% TEA)=3/1]/ethyl acetate=1/0 to 92/8) to give DMA4E-X1-PH12 (521.5 mg, 580 μmol, 35.6% yield, 96.1% purity) as a colorless oil. LCMS (EW58397-558-P1A2): CAD, m/z: [M+H]⁺=865.1. HRMS (ESI) m/z: [M+H]⁺ Calcd for C₅₄H₁₀₆NO₆⁺ 864.8015, found 864.8048. ¹H NMR (400 MHz, CHLOROFORM-d, EW58397-558-P1A1) δ 4.86 (t, J=6.4 Hz, 1H), 4.06 (t, J=6.8 Hz, 4H), 2.36-2.24 (m, 8H), 2.22 (s, 6H), 1.71-1.45 (m, 18H), 1.43-1.19 (m, 42H), 1.18-1.02 (m, 8H), 0.87 (d, J=6.8 Hz, 12H), 0.85 (d, J=6.8 Hz, 6H).

FIG. 8E is a chemical synthesis of a compound designated SM-X₁-PH12A.

To a solution of compound 1 (6.52 g, 29.2 mmol, 1.50 eq) in DCM (50 mL) was added EDCI (11.2 g, 58.5 mmol, 3.00 eq), DMAP (9.53 g, 78.0 mmol, 4.00 eq), compound 2 (5.00 g, 19.5 mmol, 1.00 eq) and DIEA (5.04 g, 39.0 mmol, 6.79 mL, 2.00 eq). The mixture was stirred at 40° C. for 12 hrs. The reaction mixture was concentrated under reduced pressure to remove solvent. The residue was purified by column chromatography (SiO₂, petroleum ether/ethyl acetate=1/0 to 99/1) to give compound 3 (2.6 g, 5.63 mmol, 28.9% yield) as a colorless oil. ¹H NMR (400 MHz, CHLOROFORM-d, EW58397-517-P1A1) δ 4.87 (t, J=6.4 Hz, 1H), 3.53 (t, J=6.8 Hz, 2H), 2.29 (t, J=7.6 Hz, 2H), 1.82-1.71 (m, 2H), 1.68-1.59 (m, 2H), 1.55-1.47 (m, 4H), 1.47-1.39 (m, 2H), 1.37-1.22 (m, 28H), 0.89 (t, J=6.8 Hz, 6H).

To a solution of compound 3 (2.50 g, 5.42 mmol, 1.00 eq) in EtOH (30 mL) was added compound 4 (993 mg, 16.3 mmol, 981 μL, 3.00 eq) and KI (899 mg, 5.42 mmol, 1.00 eq). The mixture was stirred at 60° C. for 12 hrs. The reaction mixture was concentrated under reduced pressure to remove solvent. The residue was purified by column chromatography (SiO₂, DCM/MeOH=1/0 to 10/1) to give compound 5 (1.50 g, 2.90 mmol, 53.5% yield) as a colorless oil. 1H NMR (400 MHz, CHLOROFORM-d, EW58397-520-P1A1) δ 4.86 (q, J=6.4 Hz, 1H), 3.92-3.73 (m, 2H), 2.94 (s, 2H), 2.77 (s, 2H), 2.28 (t, J=7.6 Hz, 2H), 1.68-1.56 (m, 4H), 1.55-1.43 (m, 4H), 1.40-1.16 (m, 30H), 0.88 (t, J=6.8 Hz, 6H).

To a solution of compound 6 (921 mg, 4.72 mmol, 1.10 eq) in DCM (10 mL) was added EDCI (988 mg, 5.15 mmol, 1.20 eq), Core A (800 mg, 4.29 mmol, 1.00 eq) and DMAP (420 mg, 3.43 mmol, 0.80 eq). The mixture was stirred at 25° C. for 12 hrs. The reaction mixture was concentrated under reduced pressure to remove solvent. The residue was purified by column chromatography (SiO₂, petroleum ether/ethyl acetate=1/0 to 99/1) to give compound 7 (1.70 g, 4.29 mmol, 99.8% yield) as a colorless oil. ¹H NMR (400 MHz, CHLOROFORM-d, EW58397-533-P1A1) δ 4.07 (t, J=6.8 Hz, 2H), 3.41 (t, J=6.8 Hz, 2H), 2.33 (t, J=7.6 Hz, 2H), 1.89 (q, J=7.6 Hz, 2H), 1.72-1.45 (m, 8H), 1.40-1.21 (m, 6H), 1.18-1.06 (m, 4H), 0.87 (d, J=6.4 Hz, 6H), 0.86 (d, J=6.4 Hz, 3H).

To a solution of compound 7 (1.48 g, 4.07 mmol, 1.20 eq) in ACN (8 mL) and CPME (8 mL) was added KI (620 mg, 3.74 mmol, 1.10 eq), compound 5 (1.50 g, 3.40 mmol, 1.00 eq) and K₂CO₃ (1.88 g, 13.6 mmol, 4.00 eq). The mixture was stirred at 70° C. for 12 hrs. The reaction mixture was concentrated under reduced pressure to remove solvent. The residue was purified by column chromatography (SiO₂, [petroleum ether/DCM (0.1% TEA)=3/1]/ethyl acetate=1/0 to 96/4) to give SM-X₁-PH12A

(508.2 mg, 683 μmol, 20.1% yield, 97.4% purity) as a yellow oil. LCMS (EW58397-537-P1A1): CAD, m/z: [M+H]⁺=724.9. HRMS (ESI) m/z: [M+H]⁺ Calcd for C₄₅H₉₀NO₅⁺ 724.6814, found 724.6843. ¹H NMR (400 MHz, CHLOROFORM-d, EW58397-537-P1A1) δ 4.87 (q, J=6.0 Hz, 1H), 4.07 (t, J=6.8 Hz, 2H), 3.53 (t, J=5.2 Hz, 2H), 2.58 (t, J=5.2 Hz, 2H), 2.51-2.40 (m, 4H), 2.37-2.22 (m, 4H), 1.71-1.56 (m, 6H), 1.55-1.39 (m, 10H), 1.36-1.22 (m, 38H), 1.17-1.02 (m, 4H), 0.87 (q, J=6.8 Hz, 15H).

FIG. 8F is a chemical synthesis of compounds designated SM-X₁—PH12B and ALC-PHY12A.

To a solution of compound 1 (4.81 g, 18.1 mmol, 1.30 eq) in DCM (20 mL) was added KC3-X1-PH12_4 (Core A, FIG. 8A) (2.60 g, 14.0 mmol, 1.00 eq), DMAP (1.36 g, 11.2 mmol, 0.80 eq) and EDCI (3.21 g, 16.7 mmol, 1.20 eq). The mixture was stirred at 25° C. for 12 hrs. The mixture was concentrated in vacuo to give a residue. The residue was purified by flash silica gel chromatography (ISCO®; 40 g SepaFlash® Silica Flash Column, eluent of 0%~3% ethyl acetate/petroleum ether gradient @60 mL/min) to give compound 2 (4.7 g, 9.87 mmol, 70.7% yield) as a colorless oil.

¹H NMR (400 MHz, CHLOROFORM-d, EW58311-628-P1A1) δ 4.07 (t, J=6.8 Hz, 2H), 3.41 (t, J=6.8 Hz, 2H), 2.30 (t, J=7.6 Hz, 2H), 1.86 (q, J=7.2 Hz, 2H), 1.67-1.49 (m, 6H), 1.44-1.24 (m, 18H), 1.18-1.06 (m, 4H), 0.87 (d, J=6.8 Hz, 6H), 0.86 (d, J=6.4 Hz, 3H).

To a solution of compound 2 (2.00 g, 4.61 mmol, 2.20 eq) in MeCN (20 mL) was added KI (801 mg, 4.82 mmol, 2.30 eq), K₂CO₃ (1.10 g, 7.97 mmol, 3.80 eq) and compound 3 (128 mg, 2.10 mmol, 127 μL, 1.00 eq). The mixture was stirred at 50° C. for 48 hrs. The reaction was concentrated in vacuum to give a residue. The residue was purified by prep-HPLC (column: PHS XP Phenyl-Hexyl 150*25 mm*7 um; mobile phase: [H₂O (0.225% FA)-EtOH]; gradient: 60%-90% B over 15.0 min). SM-X₁—PH12B (652.9 mg, 785 μmol, 30.1% yield, 97.7% purity, FA) was obtained as a colorless oil. LCMS (EW58311-638-P1C1): CAD, m/z, [M+H]⁺=766.9. HRMS (ESI) m/z: [M+H]⁺ Calcd for C₄₈H₉₆NO₅⁺ 766.7283, found 766.7316. ¹H NMR (400 MHz, CHLOROFORM-d, EW58311-638-P1A2) δ 8.56 (s, 0.37H), 4.07 (t, J=6.8 Hz, 4H), 3.83 (t, J=4.8 Hz, 2H), 2.94 (t, J=4.8 Hz, 2H), 2.88-2.81 (m, 4H), 2.30 (t, J=7.6 Hz, 4H), 1.69-1.48 (m, 14H), 1.42-1.20 (m, 38H), 1.18-1.05 (m, 8H), 0.87 (d, J=6.8 Hz, 12H), 0.86 (d, J=6.4 Hz, 4H).

To a solution of compound 1 (2.47 g, 5.70 mmol, 2.20 eq) in MeCN (30 mL) was added K₂CO₃ (895 mg, 6.47 mmol, 2.50 eq), compound 2 (231 mg, 2.59 mmol, 240 μL, 1.00 eq) and KI (215 mg, 1.29 mmol, 0.50 eq). The mixture was stirred at 60° C. for 12 hrs. The reaction mixture was cooled to 20° C. and concentrated under reduced pressure to remove solvent. The residue was purified by column chromatography (SiO₂, [petroleum ether/DCM (0.1% TEA)=3/1]/ethyl acetate=1/0 to 91/9) to give ALC-PHY12A (537.7 mg, 677 μmol, 26.9% yield, 95.6% purity) as a colorless oil. LCMS (EW58397-551-P1B1): CAD, m/z: [M+H]⁺=795.0. HRMS (ESI) m/z: [M+H]⁺ Calcd for C₅₀H₁₀₀NO₅⁺ 794.7596, found 794.7628. ¹H NMR (400 MHz, CHLOROFORM-d, EW58397-551-P1B1) δ4.06 (t, J=6.8 Hz, 4H), 3.54 (t, J=5.2 Hz, 2H), 2.43 (t, J=8.4 Hz, 6H), 2.29 (t, J=7.6 Hz, 4H), 1.70-1.56 (m, 12H), 1.55-1.43 (m, 6H), 1.40-1.21 (m, 38H), 1.18-1.06 (m, 8H), 0.87 (t, J=6.8 Hz, 12H), 0.85 (t, J=6.4 Hz, 6H).

FIG. 8G is a chemical synthesis of a compound designated DODMA-X1-PH5.

To a solution of compound 1 (1.70 g, 9.26 mmol, 1.00 eq, HCl) in DMF (30 mL) was added NaH (3.70 g, 92.6 mmol, 60.0% purity, 10.0 eq) at 0° C. under N₂ atmosphere. After stirring for 30 mins under N₂ atmosphere, a solution of compound 2 (4.56 g, 22.2 mmol, 2.40 eq) and 1,4,10-trioxa-7,13-diazacyclopentadecane (404 mg, 1.85 mmol, 0.20 eq) was added under N₂ atmosphere. The mixture was stirred at 25° C. for 12 hrs under N₂ atmosphere. The reaction mixture was cooled to 0° C. and poured into ice-H₂O (50 mL) under N₂ atmosphere. The aqueous layer was extracted DCM (30 mL*3). The combined organic layers were washed with brine (30 mL*3), dried over Na₂SO₄, filtered and concentrated under reduced pressure. The residue was purified by column chromatography (SiO₂, PE/ethyl acetate=20/1 to 10/1) to give compound 3 (1.10 g, 2.22 mmol, 24.0% yield, 80.0% purity) as a colorless oil. ¹H NMR (400 MHz, CHLOROFORM-d, EW53829-930-P1A) δ 5.88-5.73 (m, 2H), 4.99 (dd, J=17.2, 1.6 Hz, 2H), 4.93 (d, J=10.0 Hz, 2H), 3.59 (td, J=9.2, 6.8 Hz, 1H), 3.49-3.32 (m, 6H), 2.36-2.23 (m, 4H), 2.22 (s, 6H), 2.04 (q, J=6.8 Hz, 4H), 1.65-1.45 (m, 8H), 1.41-1.36 (m, 4H), 1.35-1.30 (m, 10H).

To a solution of compound 3 (1.10 g, 2.78 mmol, 1.00 eq) in AcOH (22.0 mL) was added KMnO₄ (1.76 g, 11.1 mmol, 4.00 eq) in H₂O (100 mL). The mixture was stirred at 25° C. for 3 hrs. The reaction was quenched by the addition of Na₂S₂O₃ (2.20 g) and NaHSO₃ (1.20 g) at room temperature, filtered and concentrated under reduced pressure to give a residue. The residue was purified by prep-MPLC (FA condition, Irregular C18, 40-63 μm, 60A) to give compound 4 (300 mg, 660 μmol, 25.9% yield, 95.0% purity) as a colorless oil. ¹H NMR (400 MHz, CHLOROFORM-d, EW53829-945-P1A) 63.60 (dt, J=8.8, 6.4 Hz, 1H), 3.50-3.30 (m, 6H), 3.03-2.82 (m, 2H), 2.72 (s, 6H), 2.30 (s, 4H), 1.93-1.71 (m, 2H), 1.67-1.48 (m, 10H), 1.42-1.29 (m, 12H).

To a solution of compound 4 (150 mg, 348 μmol, 1.00 eq) and DODAP-X1-PH5_8a (KC3-X1-PH1_2, FIG. 4A) (238 mg, 1.04 mmol, 3.00 eq) in DCM (5.00 mL) was added EDCI (267 mg, 1.39 mmol, 4.00 eq), DIEA (449 mg, 3.48 mmol, 605 μL, 10.0 eq) and DMAP (106 mg, 869 μmol, 2.50 eq). The mixture was stirred at 40° C. for 10 hrs. The reaction mixture was concentrated under reduced pressure to remove solvent. The residue was purified by column chromatography (SiO₂, [PE/DCM (5/1) (0.1% TEA)]/ethyl acetate=30/1 to 10/1) to give DODMA-X1-PH5 (146.5 mg, 170 μmol, 49.0% yield, 99.4% purity) as a colorless oil. LCMS (EW53829-950-P1A): CAD, m/z: [M+H]⁺=853.0. HRMS (ESI) m/z: [M+H]⁺ Calcd for C₅₃H₁₀₆NO₆⁺ 852.8015, found 852.8047. ¹H NMR (400 MHz, CHLOROFORM-d, EW53829-950-P1A) δ 4.17-4.00 (m, 4H), 3.59 (td, J=9.2, 6.4 Hz, 1H), 3.49-3.28 (m, 6H), 2.32-2.25 (m, 6H), 2.23 (s, 6H), 1.69-1.47 (m, 20H), 1.40-1.20 (m, 28H), 1.18-1.04 (m, 10H), 0.94-0.77 (m, 24H).

FIG. 8H is a chemical synthesis of compounds designated MA-X2-PH12 and MAE-X2-PH12.

To a solution of compound 1 (4.00 g, 16.9 mmol, 1.00 eq) in DCM (50.0 mL) was added Core B (KC3-X2-PH11_4, FIG. 7A) (3.61 g, 16.9 mmol, 1.00 eq), DMAP (1.65 g, 13.5 mmol, 0.80 eq) and EDCI (3.88 g, 20.2 mmol, 1.20 eq). The mixture was stirred at 25° C. for 12 hrs. The mixture was concentrated in vacuo to give a residue. The residue was purified by flash silica gel chromatography (ISCO®; 80 g SepaFlash® Silica Flash Column, eluent of 0~3% ethylacetate/petroleum ether gradient at 60 mL/min) to give compound 2 (6.40 g, 14.8 mmol, 87.5% yield) as a colorless oil. ¹H NMR (400 MHz, CHLOROFORM-d, EW58311-673-P1A1) δ4.06 (t, J=6.8 Hz, 2H), 3.41 (t, J=6.8 Hz, 2H), 2.30 (t, J=7.6 Hz, 2H), 1.86 (q, J=7.2 Hz, 2H), 1.67-1.48 (m, 6H), 1.44-1.25 (m, 18H), 1.18-1.03 (m, 4H), 0.87 (d, J=6.4 Hz, 6H), 0.85 (d, J=6.8 Hz, 3H).

To a solution of compound 2 (2.84 g, 6.56 mmol, 3.00 eq) in ACN (15.0 mL) was added (4-methoxyphenyl)methanamine (300 mg, 2.19 mmol, 284 μL, 1.00 eq), K₂CO₃ (907 mg, 6.56 mmol, 3.00 eq), NaI (65.6 mg, 437 μmol, 0.20 eq) and Cs₂CO₃ (713 mg, 2.19 mmol, 1.00 eq). The mixture was stirred at 80° C. for 12 hrs. The mixture was concentrated in vacuo to give a residue. The residue was purified by flash silica gel chromatography (ISCO®; 40 g SepaFlash® Silica Flash Column, eluent of 0~3% ethylacetate/petroleum ether gradient at 60 mL/min) to give compound 3 (1.50 g, 1.07 mmol, 48.9% yield) as a colorless oil. ¹H NMR (400 MHz, CHLOROFORM-d, EW58311-677-P1A1) δ7.23 (d, J=8.4 Hz, 2H), 6.84 (d, J=8.8 Hz, 2H), 4.06 (t, J=6.8 Hz, 4H), 3.81 (s, 3H), 3.48 (s, 2H), 2.37 (t, J=7.2 Hz, 4H), 2.30 (t, J=7.6 Hz, 4H), 1.65-1.46 (m, 14H), 1.44-1.25 (m, 38H), 1.16-1.02 (m, 8H), 0.87 (d, J=6.8 Hz, 12H), 0.85 (d, J=6.4 Hz, 6H).

To a solution of compound 3 (1.50 g, 1.78 mmol, 1.00 eq) in EtOAc (20.0 mL) and AcOH (5.00 mL) was added Pd(OH)₂/C (250 mg, 356 μmol, 20.0% purity, 0.20 eq) under N₂ atmosphere. The mixture was stirred at 25° C. for 12 hrs under H₂ (45 psi). Filtered with diatomaceous earth, EA (50 mL) was added to rinse filter cake, and the filtrate was concentrated under reduced pressure to give a mixture. The mixture was quenched by addition sat.Na₂CO₃ (50 mL) at 25° C., and extracted with EtOAc (20 mL*3). The combined organic layers were washed with brine (20 mL*3), dried over Na₂SO₄, filtered and concentrated under reduced pressure to give a residue. The residue was purified by flash silica gel chromatography (ISCO®; 40 g SepaFlash® Silica Flash Column, eluent of 0~2% ethyl acetate/petroleum ether gradient at 60 mL/min) to give compound 4 (1.30 g, 1.04 mmol, 58.5% yield) as a white solid. ¹H NMR (400 MHz, CHLOROFORM-d, EW58311-683-P1A1) δ4.06 (t, J=6.8 Hz, 4H), 2.59 (t, J=7.6 Hz, 4H), 2.30 (t, J=7.6 Hz, 4H), 1.67-1.46 (m, 16H), 1.35-1.24 (m, 37H), 1.18-1.05 (m, 8H), 0.87 (d, J=6.8 Hz, 12H), 0.85 (d, J=6.8 Hz, 6H).

To a solution of compound 4 (1.20 g, 1.66 mmol, 1.00 eq) and compound 5 (557 mg, 3.32 mmol, 2.00 eq) in DCM (20.0 mL) was added EDCI (382 mg, 1.99 mmol, 1.20 eq) and DMAP (162 mg, 1.33 mmol, 0.80 eq). The mixture was stirred at 25° C. for 12 hrs. The reaction was concentrated in vacuo to give a residue. The residue was purified by flash silica gel chromatography (ISCO®; 20 g SepaFlash® Silica Flash Column, eluent of 0~5% ethyl acetate/(petroleum ether/dichloromethane=3/1, 0.1% TEA as eluant) at 40 mL/min). Then purified by Prep-HPLC (column: PHS XP Phenyl-Hexyl 150*25 mm*7 μm; mobile phase: [H₂O (0.225% FA)-EtOH]; gradient: 60%-90% B over 15.0 min) and (column: PHS XP Phenyl-Hexyl 150*25 mm*7 um; mobile phase: [ACN-EtOH]; gradient: 10%-40% B over 15.0 min) to give MAE-X2-PH12 (543.6 mg, 650 mol, 38.8% yield, 99.9% purity) as a faint yellow oil. LCMS (EW58311-693-P1C1): CAD, m/z: [M+H]⁺=836.1. HRMS (ESI) m/z: [M+H]⁺ Calcd for C₅₂H₁₀₃N₂O₅⁺ 835.78 62, found 835.7894. ¹H NMR (400 MHz, CHLOROFORM-d, EW58311-693-P1A2) δ 4.06 (td, J=6.8, 2.8 Hz, 4H), 3.29 (d, J=7.6 Hz, 2H), 3.21 (d, J=7.2 Hz, 2H), 2.40 (t, J=7.2 Hz, 2H), 2.36-2.27 (m, 12H), 1.85 (q, J=7.2 Hz, 2H), 1.67-1.47 (m, 14H), 1.40-1.21 (m, 38H), 1.17-1.03 (m, 8H), 0.87 (d, J=6.4 Hz, 12H), 0.85 (d, J=6.8 Hz, 6H).

To a solution of compound 2 (2.00 g, 4.61 mmol, 1.00 eq) in ACN (30.0 mL) was added DIEA (1.13 g, 8.72 mmol, 1.52 mL, 1.89 eq) and MeNH₂/THF (2.00 M, 1.85 mL, 0.80 eq). The mixture was stirred at 75° C. for 16 hrs. The reaction was concentrated in vacuum to give a residue. The residue was purified by flash silica gel chromatography (ISCO®; 40 g SepaFlash® Silica Flash Column, eluent of 0~8% ethyl acetate/(petroleum ether/dichloromethane=3/1, 0.1% TEA as eluant) at 40 mL/min). Then purified by two Prep-HPLC (column: PHS XP Phenyl-Hexyl 150*25 mm*7 m; mobile phase: [H₂O (0.225% FA)-EtOH]; gradient: 58%-88% B over 15.0 min) to give MA-X2-PH12 (279.86 mg, 372 mol, 22.8% yield, 97.2% purity) as a colorless oil. LCMS (EW58311-688-P1A3): CAD, m/z: [M+H]⁺=736.9. HRMS (ESI) m/z: [M+H]⁺ Calcd for C₄₇H₉₄NO₄⁺ 736.717 8, found 736.7207. ¹H NMR (400 MHz, CHLOROFORM-d, EW58311-688-P1A2) δ 4.06 (t, J=6.8 Hz, 4H), 2.44-2.20 (m, 11H), 1.68-1.47 (m, 14H), 1.38-1.20 (m, 38H), 1.17-1.05 (m, 8H), 0.87 (d, J=6.4 Hz, 12H), 0.85 (d, J=6.8 Hz, 6H).

FIG. 8I is a chemical synthesis of a compound designated DMA3E-X2-PH12. To a solution of KC3-X2-PH12_4 (Compound 4, FIG. 7A) (5.00 g, 6.80 mmol, 1.00 eq) in THF (50 mL) was added NaBH₄ (720 mg, 19.0 mmol, 2.80 eq) and EtOH (940 mg, 20.4 mmol, 3.00 eq). The mixture was stirred at 30-40° C. for 12 hrs under N₂ atmosphere. The reaction mixture was quenched by addition NH₄Cl (100 mL) at 25° C. under N₂ atmosphere and extracted with EtOAc (30 mL*3). The combined organic layers were washed with brine (30 mL*3), dried over Na₂SO₄, filtered and concentrated under reduced pressure to give a residue. The residue was purified by flash silica gel chromatography (ISCO®; 40 g SepaFlash® Silica Flash Column, eluent of 0%~4% ethyl acetate/petroleum ether gradient at 60 mL/min) to give compound 1 (4.00 g, 5.43 mmol, 79.8% yield) as a white solid.

¹H NMR (400 MHz, CHLOROFORM-d, EW58311-626-P1A1) δ 4.08-4.04 (m, 4H), 3.64-3.51 (m, 1H), 2.30 (t, J=7.6 Hz, 4H), 1.65-1.56 (m, 10H), 1.38-1.24 (m, 46H), 1.16-1.05 (m, 8H), 0.87 (d, J=6.4 Hz, 12H), 0.85 (d, J=6.4 Hz, 6H).

To a solution of compound 1 (1.00 g, 1.36 mmol, 1.00 eq) and compound 2 (214 mg, 1.63 mmol, 1.20 eq) in DCM (10 mL) was added EDCI (312 mg, 1.63 mmol, 1.20 eq) and D MAP (133 mg, 1.09 mmol, 0.80 eq). The mixture was stirred at 25° C. for 12 hrs. The reaction was concentrated in vacuum to give a residue. The residue was purified by flash silica gel chrom atography (ISCO®; 12 g SepaFlash® Silica Flash Column, eluent of 0%~10% ethyl acetate/(petroleum ether/dichloromethane=3/1, 0.1% TEA as eluant) at 40 mL/min) and followed by prep-HPLC (column: PHS XP Phenyl-Hexyl 150*25 mm*7 μm; mobile phase: [H₂O (0.225% FA)-EtOH]; gradient: 62%-92% B over 15.0 min). DMA3E-X2-PH12 (103.16 mg, 118 μmol, 20.1% yield, 97.3% purity) was obtained as a colorless oil. LCMS (EW58311-635-P1C1): CAD, m/z, [M]+H]+=851.0. HRMS (ESI) m/z: [M+H]⁺ Calcd for C53H104NO6+ 850.7858, found 850.7890. ¹H NMR (400 MHz, CHLOROFORM-d, EW58311-635-P1A1) δ 8.51 (s, 0.14H), 4.87 (q, J=6.0 Hz, 1H), 4.06 (t, J=6.8 Hz, 4H), 2.43-2.27 (m, 14H), 1.84 (q, J=7.6 Hz, 2H), 1.65-1.48 (m, 14H), 1.38-1.20 (m, 42H), 1.18-1.05 (m, 8H), 0.87 (d, J=6.8 Hz, 12H), 0.84 (d, J=6.4 Hz, 6H).

Structures and properties of the compounds synthesized with non-KC basic headgroup and the biodegradable ester bond-interrupted lipid tails of the X₁ or X₂ configuration having linear X₁ and X₂ moieties and branched hydrocarbon moieties at Y₁ and Y₂ are summarized on FIG. 8J.

### Example 2. Synthesis of KCₙ-X2- Ionizable Cationic Lipids of Formula (I) Wherein X is —(CH₂)ₓ—, W is *—C(O)—O—** and Y is a Hydrocarbon Chain with (y) Carbon Atoms

Scheme 3 is a general synthesis for compounds of Formula (I) wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear or branched hydrocarbon chains with (y) total carbons, (n) is 2, 3 or 4 and Ra and Rb are each C₁₋₄ alkyl optionally substituted with hydroxyl (i.e., “KCₙ—X2” ionizable cationic lipids). In some embodiments, X₁ and X₂ are the same, Y₁ and Y₂ are the same, and Ra and Rb are each methyl. In some embodiments, Ra is methyl and Rb is —(CH₂)₂—OH.

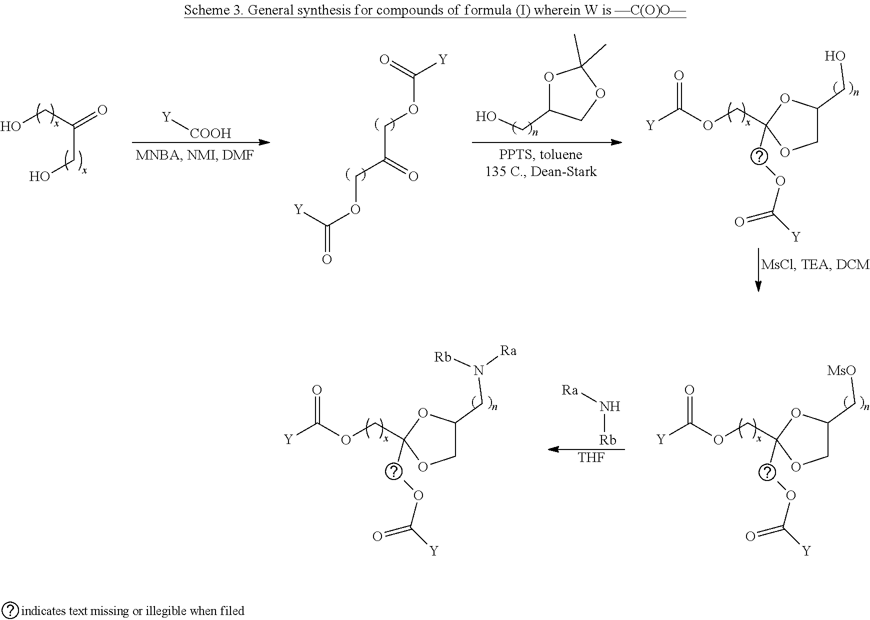

A general synthesis for compounds of formula (I) wherein W is —C(O)O— shown in Scheme 3 involves i) esterification of the dihydroxy ketone with a suitable carboxylic acid, ii) acid catalyzed trans ketal formation, iii) Mesylation of the primary alcohol, and finally iv) displacement of the mesyl group using dialkylamine resulting in a tertiary amine. A general synthesis of dihydroxy ketone starting material in Scheme 3 is presented below in Scheme 4.

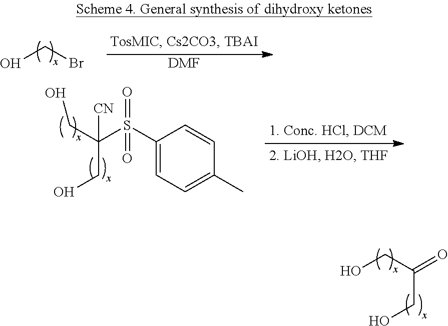

A general synthesis of dihydroxy ketone shown in Scheme 4 involves i) a suitable hydroxy alkyl bromide is treated with toluenesulfonylmethyl isocyanide in the presence of sodium hydride in DMSO, and ii) TosMIC adduct is then converted to a ketone in the presence of HCl.

The precursor and intermediate compounds for the ionizable cationic lipids can be prepared by a variety of methods including the preparation of ketone intermediates (ketonedicarboxylic acids). In addition, some precursor and intermediate compounds can be commercially obtained, such as 3-oxopentanedioic acid (3-oxoglutaric acid, acetonedicarboxylic acid CAS 542-05-2) (Millipore Sigma item No. 8.20940), 4-oxoheptanedioic acid (4-ketopimelic acid, CAS 502-50-1) (Thermo Scientific item No. 122881000), 5-oxononanedioic acid (5-oxoazelaic acid, CAS 57882-06-7) (TCI America item No. O02581). In addition, w,w′ dihydroxyketones can be obtained according to Scheme 5, an exemplary route using 6-hydroxyhexanoic acid (CAS 1191-25-9).

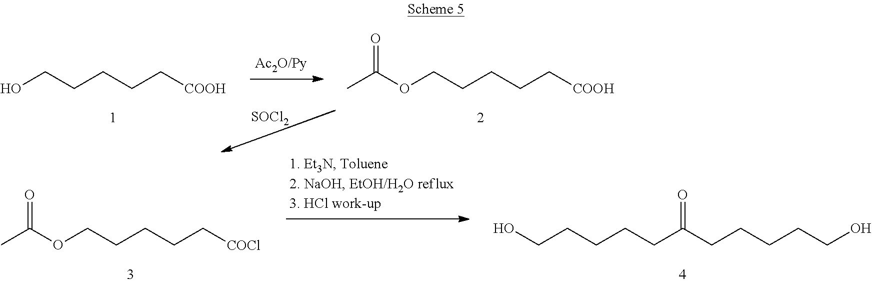

### Example 2A. KCₙ—X2 Compounds of Formula (I) Comprising Linear Alkanes at Y₁ and Y₂

The following relates to compounds of Formula (I) wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkane hydrocarbon chains with (y) total carbons.

FIG. 5A is a chemical synthesis of compound of Formula (I) designated KC3-X2-K9, wherein W₁ and W₂ are each *—C(O)—O—**, * is a covalent bond to Y₁ or Y₂ and ** is a covalent bond to X₁ or X₂ (respectively), X₁ or X₂ are each —(CH₂)₁₀—, and Y₁ or Y₂ are each —(CH₂)₁₀—CH₃.

To a solution of NaH (2.13 g, 53.35 mmol, 60% purity, 2.5 eq) in DMSO (100 mL) at 0° C. The mixture was stirred at 25° C. for 1 hr. Then added TBAI (788.21 mg, 2.13 mmol, 0.1 eq) and TosMIC. The mixture was stirred at 25° C. for 15 min. Then added compound 1A (10 g, 44.81 mmol, 2.1 eq). The mixture was stirred at 25° C. for 1 hr. The reaction mixture was added aq.NH₄Cl (sat. 200 mL), then the mixture was extracted with EtOAc (100 mL*2). The combined organic phases were washed with brine (100 mL), dried over Na₂SO₄, filtered and concentrated. The crude product was used directly for the next step without further purification, compound 2A (14 g, crude) was obtained as a yellow oil. ¹H NMR (400 MHz, CHLOROFORM-d EW53829-559-P1A) δ 7.85 (d, J=8.4 Hz, 2H), 7.41 (d, J=8.0 Hz, 2H), 3.62 (t, J=6.8 Hz, 4H), 2.48 (s, 3H), 2.05-1.78 (m, 6H), 1.61-1.49 (m, 7H), 1.40-1.19 (m, 26H).

To a solution of compound 2A (14 g, 29.18 mmol, 1 eq) in DCM (140 mL) was added HCl (12 M, 49.91 mL, 20.52 eq). The mixture was stirred at 25° C. for 1 hr. The reaction mixture was concentrated under reduced pressure to remove DCM. After the lyophilization. The residue was triturated with EtOAc (200 mL) at 25° C. for 30 min to give compound 1 (3.5 g, 10.02 mmol, 34% yield, 90% purity) as a brown solid. ¹H NMR (400 MHz, CHLOROFORM-d EW53829-560-P1A) δ 3.63 (t, J=6.8 Hz, 4H), 2.38 (t, J=7.6 Hz, 4H), 1.65-1.46 (m, 10H), 1.39-1.17 (m, 20H).

To a solution of compound 1 (2 g, 6.36 mmol, 1 eq) and compound 1B (3.18 g, 15.90 mmol, 2.5 eq) in DCM (30 mL) was added DIEA (8.22 g, 63.59 mmol, 11.08 mL, 10 eq), EDCI (4.88 g, 25.44 mmol, 4 eq) and DMAP (1.94 g, 15.90 mmol, 2.5 eq). The mixture was stirred at 40° C. for 12 hr. The reaction mixture was concentrated under reduced pressure to remove solvent. The residue was purified by column chromatography (SiO₂, PE: EtOAc=50/1 to10/1) to give compound 2 (2.4 g, 3.36 mmol, 52% yield, 95% purity) as a white solid. ¹H NMR (400 MHz, CHLOROFORM-d EW53829-563-P1A) δ 4.05 (t, J=6.8 Hz, 4H), 2.38 (t, J=7.4 Hz, 4H), 2.29 (t, J=7.6 Hz, 4H), 1.68-1.51 (m, 15H), 1.27 (m, 54H), 0.88 (t, J=6.8 Hz, 6H).

A mixture of compound 2 (2.4 g, 3.53 mmol, 1 eq), compound 3 (1.13 g, 7.07 mmol, 2 eq) and PPTS (444.06 mg, 1.77 mmol, 0.5 eq) in Tol. (30 mL) was heated to reflux and remove water by Dean-Stark trap. The mixture was stirred at 135° C. for 12 hr. The reaction mixture was quenched by addition aq.NaHCO₃ (sat. 20 mL) at 0° C., and then diluted with ice-water 30 mL and extracted with EtOAc (30 mL*3). The combined organic layers were washed with H₂O (50 mL*2), dried over Na₂SO₄, filtered and combined organic concentrated under reduced pressure to give a residue. The residue was purified by column chromatography (SiO₂, PE: EtOAc=10/1 to 5/1) to give compound 4 (1.5 g, 1.73 mmol, 48% yield, 90% purity) as a yellow oil. ¹H NMR (400 MHz, CHLOROFORM-d EW53829-565-P1A) δ 4.06 (t, J=6.8 Hz, 6H), 3.76-3.62 (m, 2H), 3.51-3.43 (m, 1H), 2.29 (t, J=7.6 Hz, 4H), 1.75-1.53 (m, 19H), 1.28 (m, 59H), 0.89 (t, J=6.8 Hz, 6H).

To a solution of compound 4 (1.5 g, 1.92 mmol, 1 eq) in DCM (20 mL) was added TEA (971.44 mg, 9.60 mmol, 1.34 mL, 5 eq) and MsCl (840 mg, 7.33 mmol, 567.57 μL, 3.82 eq) at 0° C. The mixture was stirred at 25° C. for 2 hr. The reaction mixture was concentrated under reduced pressure to remove solvent. The residue was purified by column chromatography (SiO₂, PE (0.1% TEA)/EtOAc=10/1 to 5/1) to give compound 6 (1.5 g, 1.75 mmol, 90% yield) as a yellow oil. ¹H NMR (400 MHz, CHLOROFORM-d EW53829-567-P1A) δ 4.38-4.19 (m, 2H), 4.13-3.99 (m, 6H), 3.52-3.44 (m, 1H), 3.02 (s, 3H), 2.29 (t, J=7.2 Hz, 4H), 1.73-1.50 (m, 18H), 1.39-1.21 (m, 59H), 0.89 (t, J=7.2 Hz, 7H).

A solution of compound 5 (1.4 g, 1.63 mmol, 1 eq) in Me₂NH/THF (2 M, 28.00 mL) was stirred at 50° C. for 10 hr. The reaction mixture was concentrated under reduced pressure to remove solvent. The residue was purified by column chromatography (SiO₂, PE(0.1% TEA): EtOAc=20/1 to 10/1) to give AKG-KC3-X2-K9 (520.6 mg, 624.74 μmol, 38% yield, 97% purity) as a yellow oil. ¹H NMR (400 MHz, CHLOROFORM-d EW53829-569-P1A1) δ 4.17-3.95 (m, 6H), 3.55-3.35 (m, 1H), 2.41-2.31 (m, 6H), 2.24 (s, 6H), 1.67-1.56 (m, 16H), 1.27 (m, 56H), 0.89 (t, J=7.2 Hz, 6H). LCMS (EW53829-569-P1A): CAD, m/z, M+H⁺=808.9. LCMS (EW53829-569-P1A1): ELSD, m/z, M+H⁺=808.9. HRMS (ESI) m/z: [M+H]⁺ Calcd for: C₅₀H₉₈NO₆⁺808.7389, found 808.7415.

FIG. 5B is a list of other exemplary compounds of Formula (I), wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkane hydrocarbon chains with (y) total carbons.

### Example 2B. KCₙ—X2 Compounds of Formula (I) Comprising Linear Alkenes at Y₁ and Y₂

The following relates to compounds of Formula (I) wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkene hydrocarbon chains with (y) total carbons.

FIG. 6A is a chemical synthesis of compound of Formula (I) designated KC3-X2-C(trans), wherein W₁ and W₂ are each *—C(O)—O—**, * is a covalent bond to Y₁ or Y₂ and ** is a covalent bond to X₁ or X₂ (respectively), X₁ or X₂ are each —(CH₂)₃—, and Y₁ or Y₂ are each —(CH₂)₂—CH═CH(CH₂)₈(CH₃)—.

A solution of compound 6B(Trans-) (3.3 g, 9.36 mmol, 1 eq) and bis[(Z)-1-tert-butyl-4,4-dimethyl-3-oxo-pent-1-enoxy]nickel (796.15 mg, 1.87 mmol, 0.2 eq) in MeOH (25 mL) was stirred at 100° C. for 12 hrs with sealed tube. The reaction mixture was concentrated under reduced pressure to remove the organic phase. The residue was purified by column chromatography (SiO₂, PE:EA=1:0 to PE:EA=50:1). Compound 6C (Trans-) (1.3 g, 5.41 mmol, 57.8% yield) was obtained as a yellow oil. ¹H NMR (CHLOROFORM-d, 400 MHz, EW53829-335-P1A): δ 5.30-5.54 (m, 2H), 3.67 (s, 3H), 2.23-2.43 (m, 4H), 1.97 (q, J=6.8 Hz, 2H), 1.21-1.38 (m, 14H), 0.88 (t, J=6.8 Hz, 3H).

A solution of compound 6C(Trans-) (1.2 g, 4.99 mmol, 1 eq) and LiOH·H₂O (418.94 mg, 9.98 mmol, 2 eq) in THF (30 mL) and H₂O (10 mL) was stirred at 20° C. for 12 hrs. The reaction was concentrated in vacuum, the mixture was adjusted to pH=5 with HCl aq. (1M), the solution was extracted with EA (50 mL×2), the organic phase was washed with brine (50 mL), dried with Na₂SO₄. The mixture was filtered and concentrated in vacuum. Compound 6(Trans-) (1.1 g, crude) was obtained as yellow oil. 1H NMR (CHLOROFORM-d, 400 MHz, EW53829-337-P1A): δ 5.37 (dt, J=19.2, 6.2 Hz, 2H), 2.31-2.38 (m, 2H), 2.18-2.29 (m, 2H), 1.90 (q, J=6.4 Hz, 2H), 1.14-1.31 (m, 15H), 0.81 (t, J=6.8 Hz, 3H). ¹³C NMR (CHLOROFORM-d, EW53829-337-P1A): δ 179.6, 132.2, 127.5, 90.8, 39.5, 34.2, 32.5, 31.9, 29.5, 29.2, 27.6, 27.4, 26.2, 22.7, 14.1.

To a solution of KC3-X2-C_7 (608.30 mg, 2.21 mmol, 1 eq) in DCM (20 mL) was added NMI (906.77 mg, 11.04 mmol, 880.35 μL, 5 eq), (2-methyl-6-nitro-benzoyl) 2-methyl-6-nitro-benzoate (2.28 g, 6.63 mmol, 3 eq) and compound 6(Trans-) (1 g, 4.42 mmol, 2 eq). The mixture was stirred at 40° C. for 10 hr. The reaction mixture was concentrated under reduced pressure to remove solvent. The residue was purified by column chromatography (SiO₂, Petroleum ether, 0.1% TEA/Ethyl acetate=20/1 to 10/1). KC3-X2-C(Trans-) (314.8 mg, 441.23 μmol, 61.1% yield, 97% purity) was obtained as a colorless oil. 1H NMR (400 MHz, CHLOROFORM-d, EW53829-361-P1A) δ 5.52-5.33 (m, 4H), 4.15-4.02 (m, 6H), 3.46 (s, 1H), 2.39-2.26 (m, 10H), 2.23 (s, 6H), 1.97 (q, J=6.6 Hz, 4H), 1.78-1.43 (m, 13H), 1.40-1.17 (m, 29H), 0.88 (t, J=6.8 Hz, 6H). ¹³C NMR (CHLOROFORM-d, EW53829-361-P1A) δ 173.31, 173.31, 131.91, 131.91, 127.91, 127.91, 111.14, 70.22, 64.44, 64.39, 59.56, 45.46, 34.46, 34.21, 34.02, 32.58, 31.96, 31.27, 29.65, 29.58, 29.53, 29.39, 29.21, 28.01, 24.09, 23.46, 23.09, 22.74, 14.17. LC MS (EW58397-326-P1B2): CAD m/z, M+H⁺=692.7. Special MS (EW58397-326-P1B):ELSD m/z, M+H⁺=692.8. HRMS (ESI) m/z: [M+H]⁺ Calcd for C42H78NO6+ 692.5824, found 692.5854.

FIG. 6B is a list of other exemplary compounds of Formula (I), wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical linear alkene hydrocarbon chains with (y) total carbons.

### Example 2C. KCₙ—X2 Compounds of Formula (I) Comprising Branched Alkanes at Y₁ and Y₂

The following relates to compounds of Formula (I) wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical branched alkane hydrocarbon chains with (y) total carbons.

FIG. 7A is a chemical synthesis of compound of Formula (I) designated KC3-X2-PH12 wherein W₁ and W₂ are each *—C(O)—O—**, * is a covalent bond to Y₁ or Y₂ and ** is a covalent bond to X₁ or X₂ (respectively), X₁ or X₂ are each —(CH₂)₁₀—, and Y₁ or Y₂ are each —(CH₂)-(2 MB)₂ wherein 2 MB is a head-to-tail 2-methylbut-2-ene (2 MB) monomer unit.

A solution of NaH (5.90 g, 147.51 mmol, 60% purity, 2.4 eq) in DMSO (300 mL) was stirred at 25° C. for 1 hr. Then TBAI (2.27 g, 6.15 mmol, 0.1 eq) and Tosmic (12 g, 61.46 mmol, 1 eq) was added. The mixture was stirred at 25° C. for 15 min. After the addition of compound 1 (30.61 g, 129.07 mmol, 2.1 eq), the mixture was stirred at 25° C. for 1 hr. The reaction mixture was quenched by addition sat-NH₄Cl (aq.200 mL) at 25° C., and then diluted with ice-water (100 mL) and extracted with EtOAc (200 mL*3). The combined organic layers were washed with brine (200 mL*3), dried with anhydrous Na₂SO₄, filtered and concentrated in vacuum. The crude product was used directly for the next step without further purification. Compound 2 (32 g, crude) was obtained as a brown oil.

To a solution of compound 2 (32 g, 63.02 mmol, 1 eq) in DCM (210 mL) was added con.HCl (70 mL). The mixture was stirred at 25° C. for 2 hr. The reaction mixture was concentrated under reduced pressure to remove the solvent and then lyophilized. The crude product was triturated with (PE:EtOAc=1:1) at 0° C. for 30 min to give compound 3 (18.9 g, crude) as a yellow solid. 1H NMR (400 MHz, CHLOROFORM-d, EW58397-468-P1A1) δ 3.63 (t, J=6.8 Hz, 4H), 3.38 (t, J=6.8 Hz, 2H), 2.38 (t, J=7.2 Hz, 4H), 1.58-1.52 (m, 8H), 1.36-1.25 (m, 24H).

To a solution of compound 3 (1.6 g, 4.67 mmol, 1 eq) in DCM (20 mL) was added EDCI (3.58 g, 18.68 mmol, 4 eq), DIEA (6.04 g, 46.71 mmol, 8.14 mL, 10 eq), KC3-X2-PH11_4 (2.00 g, 9.34 mmol, 2 eq) and DMAP (1.43 g, 11.68 mmol, 2.5 eq). The mixture was stirred at 40° C. for 16 hr. The reaction mixture was concentrated in vacuum. The residue was purified by column chromatography (SiO₂, Petroleum ether/Ethyl acetate=1/0 to 94/6) to give compound 4 (1.8 g, 2.41 mmol, 51% yield) as a yellow oil. ¹H NMR (400 MHz, CHLOROFORM-d, EW58397-474-P1A1) δ 4.05 (t, J=6.8 Hz, 4H), 2.38 (t, J=7.2 Hz, 4H), 2.30 (t, J=7.4 Hz, 4H), 1.65-1.50 (m, 14H), 1.41-1.21 (m, 38H), 1.17-1.02 (m, 8H), 0.90-0.80 (m, 18H).

A solution of compound 4 (2 g, 2.72 mmol, 1 eq), PPTS (205.08 mg, 816.09 μmol, 0.3 eq) and compound 5 (871.64 mg, 5.44 mmol, 2 eq) in Tol. (30 mL) was heated to reflux and the water was removed by Dean-Stark trap. The mixture was stirred at 135° C. for 16 hr. The reaction mixture was quenched by addition sat-NaHCO₃ (aq. 80 mL) at 25° C., and extracted with EtOAc (30 mL*3).

The combined organic layers were washed with brine (20 mL*3), dried with anhydrous Na₂SO₄, filtered and concentrated in vacuum. The residue was purified by flash silica gel chromatography (ISCO®; 40 g SepaFlash® Silica Flash Column, Eluent of 0~10% Ethyl acetate/Petroleum ether gradient @60 mL/min) to give compound 6 (850 mg, 832.40 μmol, 31% yield) as a yellow oil. ¹H NMR (400 MHz, CHLOROFORM-d, EW58311-476-P1A1) δ 4.12-4.03 (m, 6H), 3.72-3.64 (m, 2H), 3.52-3.43 (m, 1H), 2.32-2.28 (m, 4H), 1.64-1.56 (m, 18H), 1.35-1.24 (m, 42H), 1.16-1.05 (m, 8H), 0.88-0.83 (m, 18H).

To a solution of compound 6 (840 mg, 1.00 mmol, 1 eq) in DCM (10 mL) was added TEA (507.56 mg, 5.02 mmol, 698.15 μL, 5 eq) and MsCl (270.00 mg, 2.36 mmol, 182.43 μL, 2.35 eq) at 0° C. The mixture was stirred at 0° C. for 1 hr. The reaction mixture was quenched by addition ice-water (30 mL) at 0° C., and extracted with DCM (10 mL*3). The combined organic layers were washed with brine (10 mL*3), dried with anhydrous Na₂SO₄, filtered and concentrated in vacuum. The residue was purified by flash silica gel chromatography (ISCO®; 12 g SepaFlash® Silica Flash Column, Eluent of 0-10% Ethylacetate/Petroleum ethergradient @40 mL/min) to give compound 7 (760 mg, 788.70 μmol, 79% yield,) as a yellow oil. ¹H NMR (400 MHz, CHLOROFORM-d, EW58311-477-P1A1) δ 4.33-4.22 (m, 2H), 4.09-4.01 (m, 6H), 3.53-3.42 (m, 1H), 3.02 (s, 3H), 2.30 (t, J=7.6 Hz, 4H), 1.64-1.55 (m, 20H), 1.36-1.23 (m, 40H), 1.16-1.04 (m, 8H), 0.89-0.83 (m, 18H).

A solution of compound 7 (760 mg, 830.21 μmol, 1 eq) in Me₂NH/THF (2 M, 10 mL) was stirred at 50° C. for 10 hr. The reaction mixture was concentrated under reduced pressure to remove solvent. The residue was purified by flash silica gel chromatography (ISCO®; 12 g SepaFlash® Silica Flash Column, Eluent of 0~8% Ethyl acetate/(Petroleum ether/Dichloromethane=3/1, 0.1% TEA as eluant)@40 mL/min) and followed by reversed-phase HPLC (column: PHS XP Phenyl-Hexyl 150*25 mm*7 um; mobile phase: [H₂O (0.225% FA)-ACN]; gradient: 65%-95% B over 15.0 min) to give AKG-KC3-X2-PH12 (371.1 mg, 422.87 μmol, 48% yield, 98.5% purity) as a yellow oil. LCMS (EW58311-481-P1C1): CAD, m/z, M+H=865.0. HRMS (ESI) m/z: [M+H]⁺ Calcd for C₅₄H₁₀₆NO₆⁺ 864.8015, found 864.8032. ¹H NMR (400 MHz, CHLOROFORM-d, EW58311-481-P1A1) δ 4.13-3.99 (m, 6H), 3.52-3.40 (m, 1H), 2.44-2.21 (m, 12H), 1.67-1.48 (m, 18H), 1.39-1.19 (m, 42H), 1.17-1.04 (m, 8H), 0.92-0.82 (m, 18H).

FIG. 7B is a list of other exemplary compounds of Formula (I), wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical branched alkane hydrocarbon chains with (y) total carbons.

### Example 2D. KCₙ—X2 Compounds of Formula (I) Comprising Linear Alkenes at Y₁ and Y₂

The following example relates to compounds of Formula (I) wherein W₁ and W₂ are each *—C(O)—O—**, * is a covalent bond to Y₁ or Y₂ and ** is a covalent bond to X₁ or X₂ (respectively), X₁ or X₂ are each —(CH₂)ₓ—, and Y₁ or Y₂ are each identical branched alkene hydrocarbon chains with (y) total carbons. The synthetic schemes of FIG. 4A (KC3-X1-B) and FIG. 6A (KC3-X2-C(trans) can be adapted according to the preparation of KCₙ—X2 compounds disclosed herein, to prepare compounds wherein W₁ and W₂ are each *—C(O)—O—**, * is a covalent bond to Y₁ or Y₂ and ** is a covalent bond to X₁ or X₂ (respectively), X₁ or X₂ are each —(CH₂)₁₀—, and Y₁ or Y₂ are each —(IPN)₃ wherein IPN is a head-to-tail isopentane (IPN) monomer unit. Additional ionizable cationic lipid compounds including a variety of head groups including the KCₙ head group depicted in Formula (I) and other head groups. These compounds can be prepared according to the methods disclosed herein.

### Example 3: Preparation of Lipidic Nanoparticles (LNPs) Containing mRNA

Stock solutions of each lipid are prepared. Ionizable lipids and cholesterol are weighed out in 4 mL glass vials (Thermo B7999-2) and dissolved in ethanol (Sigma-Aldrich 200 proof, RNase free) to a final concentration of 10 mM. Other lipids such as DSPC, DPPC-NH₄, and PEG-DMG are weighed out and dissolved in ethanol to a concentration of 1 mM.

Lipid mixtures for each individual LNP are prepared by adding the desired volume of each lipid stock solution to a new vial, adding ethanol if needed to achieve a final volume of 1.2 mL which includes 20% overage. For example, 1 mL of this lipid in ethanol is used when making LNPs using a lipid-to-mRNA volume ratio of 1 mL to 3 mL. If a larger batch of LNPs is required, either 2 mL lipids or 3 mL lipids in ethanol can be mixed with either 6 or 9 mL mRNA respectively.

mRNA solutions are prepared by thawing frozen mRNA (e.g. FFluc-mRNA) vials and diluting mRNA in 6.25 mM sodium acetate (pH 5.0) to a final concentration of 0.033 mg/mL, which is confirmed by measuring the mRNA concentration by UV-Vis spectroscopy using the OD to concentration conversion factor: concentration of mRNA (ug/mL)=OD260 nm×40× dilution factor. To prepare LNPs, a NanoAssemblr Benchtop microfluidic device (from Precision Nanosystems) is used. 3 mL of mRNA solution is loaded into a 3 mL disposable syringe (BD 309656) and 1 ml of lipid mixture in a 1 ml syringe (BD309659) and placed in the NanoAssemblr. LNP formation is achieved by pumping the liquid streams through a disposable microfluidics cassette at 3:1 aqueous: alcohol volume ratio at 6 mL/min mixing speed, or through a ChipShop microfluidics chip (#187) at 18 ml/min. After mixing, 3.6 mL of LNP mixture is collected, while the initial mixed volume of 0.35 mL and last 0.05 mL of mix is discarded. Ethanol is removed by buffer exchange using sequential concentration and dilution using Amicon Ultra-4 (or Amicon Ultra-15) centrifugal concentrators (100k MWCO, at 500×g).

LNPs are typically exchanged into PBS, pH 7.4 (-calcium, -magnesium, Cytiva SH30256.02) and then 10 mM Tris, pH 7.4, 10% sucrose, concentrated to 20-50 ug/mL mRNA using an Amicon-Ultra 4 (100,000 MWCO) centrifugal concentrator, sterile filtered (Thermo Nalgene 0.2 um #720-1320) into 2 mL Corning vial (#430659) or similar prior to freezing by placing the vials at −80° C.

Exemplary LNP formulations can deliver mRNA encoding any protein antigen derived from an infectious pathogen or disease that can be targeted with the immune system. Listed in Table 3A and Table 3B are a non-exhaustive list of vaccine antigens that could be encoded by mRNA and encapsulated in LNPs to comprise a vaccine using the lipids, methods, and formulations described in Examples 1-13.

TABLE 3A
Examples of antigens that could be encoded by mRNA and
encapsulated in LNPs to comprise a novel vaccine.

| Infectious | vaccine antigen | |
|---|---|---|
| pathogen/disease | description | Reference (PMID) |
| respiratory syncytial | RSVpreF (DS-Cav1) and subsequent | 24179220, 27478931, |
| virus (RSV) | iterative structural improvements. E.g. | 30849373, 37689061 |
| | sc9-10 DS-Cav1 A149C Y458C, DS- | |
| | Cav1-153-50A | |
| Influenza virus | HA, HA stabilized stem, NA, M2 | 26301691, 26303961, |
| (IAV, IBV) | | 30808695, 36356572, |
| | | 35869085, 35383176, |
| | | 3480945 |
| Lassa virus (LASV) | stabilized LASV GPC, nucleoprotein | 38177144, 32269122, |
| | | 32142546 |
| SARS-CoV-2, | spike (S) protein, nucleocapsid, | 33128877 |
| betacoronaviruses | membrane, ORF3a, ORF1ab | |
| Middle East | spike (S) glycoprotein | 28807998 |
| respiratory syndrome | | |
| coronavirus (MERS- | | |
| CoV) | | |
| Ebola virus (EBOV) | glycoprotein (GP), nucleoprotein (NP) | 32418798, 31589088, |
| | | 32234486 |
| Marburg virus | glycoprotein (GP), nucleoprotein (NP) | 31589088, 32234486 |
| (MARV) | | |
| human | F glycoprotein, interprotomer-disulfide- | 34551978, 37738240 |
| metapneumovirus | stabilized preF or postF | |
| (HMPV) | | |
| parainfluenza virus | Type 1 and type 4. Prefusion stabilized | 30420505 |
| | fusion proteins (e.g. prefusion PIV3 F) | |
| Mumps (MuV) | Fusion glycoprotein F, prefusion F | 39527740, |
| | variants, prefusion F-HN chimera | PCT/US2020/064619, |
| | | PCT WO2021119497A1 |
| Measles (MeV) | Fusion glycoprotein F, prefusion F, | PCT/US2020/064619, |
| | prefusion F-H chimera | PCT WO2021119497A1 |
| human | gp120 eOD-GT-based immunogens (e.g. | 23539181, 31515132, |
| immunodeficiency | eOD-GT8), stabilized Env trimers (e.g. | 27174988 |
| virus (HIV) | BG505 DS-SOSPI), conserved protease | |
| | cleavage sites, gp41 fusion peptide | |
| human papilloma | L1 capsid protein, E7, glycoprotein D, | 38462683, 36867683 |
| viruses (HPV) | gDE7 fusion | |
| Zika virus (ZIKV) | prM-E | 28222903 |
| Powassan virus | prM, prE | 30566864 |
| (POWV) | | |
| MPox virus | A35, B6, M1, H3 | 38366591 |
| norovirus | capsid protein VP1 | 38811605 |
| human | glycoprotein B (gB), gH, gL, UL128, | 38324766, 29456015 |
| cytomegalovirus | UL130, UL131 | |
| (HCMV) | | |
| Ebstein Barr virus | glycoprotein gp350, gp42, gH/gL | 38479361, 38926438 |
| (EBV) | (gp42/gp350) | |
| human varicella-zoster | envelope glycoprotein E (gE) | 25916341 |
| virus (VZV) | | |
| Rabies virus (RABV) | glycoprotein (G) | 37349310 |
| West Nile virus | premembrane (prM), envelope (E) | 37125778 |
| (WNV) | | |
| Dengue virus (DENV) | envelope protein (E), premembrane (prM), | 35608749 |
| | NS1, NS3, NS5, NS4B | |
| Chikungunya virus | E1, E2, E3 glycoproteins | 23670192, 38005854, |
| (CHIKV). other | | 36474012 |
| Alphaviruses of the | | |
| Togaviridae family | | |
| (RRV, ONNV, BFV, | | |
| MAYV, SINV, EEEV, | | |
| WEEV, VEEV) | | |
| Nipah virus (NiV) | glycoprotein (G) | 38726013 |
| Mycobacterium | individual Mtb proteins (e.g. ESAT-6, | 31114037, 36604540 |
| tuberculosis | CFP10, Ag85B, Esx family proteins), | |
| (tuberculosis) | synthetic fusion proteins (e.g. M72) | |
| Plasmodium | circumsporozoite protein (CPS); RTS,S; | 27355532, 33964223, |
| falciparim (malaria) | CSP-HBsAg fusion (hepatitus B surface | 37168860, 31824483, |
| | Ag); R21; Pfs25; PfGARP; CelTOS, | 28096331, 30131879, |
| | MSP10, RH5 | 39019011, 40325041, |
| | | US9821046B2, |
| | | US11096999B2, |
| Borrelia burgdorferi | outer surface protein A (OspA) | 37533256 |
| (Lyme disease) | | |
| A. americanum (alpha- | Salivary proteins (e.g. Salp-family | 34788080, 37407406 |
| gal hypersensitivity), I. | proteins, TSLPI) | |
| scapularis | | |
| cancer | cancer neoantigens | 37165196, 28678784, |
| | | 35970920, 38538867 |

TABLE 3B
Examples of antigens that could be encoded by mRNA and
encapsulated in LNPs to comprise a novel vaccine.

| Infectious | vaccine antigen | |
|---|---|---|
| pathogen/disease | description | Reference (PMID) |
| respiratory | Prefusion stabilized F (DS-Cav1) and | 24179220, 27478931, |
| syncytial | subsequent iterative structural | 30849373, 37689061 |
| virus (RSV) | improvements. (e.g. sc9-10 DS-Cav1 | |
| | A149C Y458C or DS-Cav1-153-50A) | |
| | SEQ ID NO: 9 | |
| | RSV-G antigen | |
| | SEQ ID NO: 509 | |
| | SEQ ID NO: 510 | |
| | SEQ ID NO: 511 | |
| | SEQ ID NO: 512 | |
| | SEQ ID NO: 513 | |
| | SEQ ID NO: 515 | |
| Influenza virus | HA (e.g., SEQ ID NO: 5, SEQ ID NO: 12, | 26301691, 26303961, |
| (IAV, IBV) | SEQ ID NO: 14, SEQ ID NO: 915, SEQ ID | 30808695, 36356572, |
| | NO: 905-907, and/or SEQ ID NO: 18); | 35869085, 35383176, A |
| | HA stabilized stem (Group 1 and 2); | Dosey et al. bioRxiv |
| | NA (SEQ ID NO: 37, SEQ ID NO: 38, SEQ | 2025.07.30.667762 |
| | ID NO: 39); | |
| | M2; | |
| | stabilized H5 (e.g., SEQ ID NO: 15) | |
| Lassa virus | stabilized LASV GPC; nucleoprotein; | 38177144, 32269122, |
| (LASV) | NP-GPC chimeric fusion protein (e.g., | 32142546 |
| | SEQ ID NO: 801 or SEQ ID NO: 803) | |
| human | gp120 eOD-GT-based immunogens (e.g. | 23539181, 31515132, |
| immunodeficiency | eOD-GT8), stabilized Env trimers (e.g. | 27174988, 37098956, |
| virus (HIV) | BG505 DS-SOSPI), conserved protease | 38997302, 33766848, |
| | cleavage sites, gp41 fusion peptide, HLA- | 26687358 |
| | E-restricted epitopes | |
| Mycobacterium | individual Mtb proteins (e.g. ESAT-6, | 31114037, 36604540, |
| tuberculosis | CFP10, Ag85B, Ag85A Esx family | 15187142, 24349004, |
| (tuberculosis) | proteins); | 22133873, 34795205, |
| | SEQ ID NO: 444 | 29263854, 29996082; N |
| | SEQ ID NO: 445 | Agrawal et al. 2025, |
| | SEQ ID NO: 447 | bioRxiv |
| | SEQ ID NO: 449 | 2025.09.30.679428 |
| | SEQ ID NO: 450 | |
| | synthetic fusion proteins (e.g. M72, H4, | |
| | H56, H65, H107, CysVac2, BNT164B1/2) | |
| | SEQ ID NO: 401 | |
| | SEQ ID NO: 403 | |
| | SEQ ID NO: 405 | |
| | SEQ ID NO: 407 | |
| | SEQ ID NO: 409 | |
| | SEQ ID NO: 411 | |
| | SEQ ID NO: 413 | |
| | SEQ ID NO: 415 | |
| | SEQ ID NO: 417 | |
| | SEQ ID NO: 419 | |
| | SEQ ID NO: 421 | |
| | SEQ ID NO: 423 | |
| | SEQ ID NO: 425 | |
| | SEQ ID NO: 427 | |
| | SEQ ID NO: 429 | |
| | SEQ ID NO: 431 | |
| | SEQ ID NO: 437 | |
| | SEQ ID NO: 438 | |
| | SEQ ID NO: 440 | |
| | SEQ ID NO: 442 | |
| | SEQ ID NO: 451 | |
| Plasmodium | circumsporozoite protein (CPS), RTS, S, | 27355532, 33964223, |
| falciparim | R21, CSP-HBsAg fusion (hepatitis B | 37168860, 35822551, |
| (malaria) | surface Ag); Pfs25; PfGARP; CelTOS, | 27457156, 39019011, |
| | RH5.1, RH5.2 | 39025972 |
| Dengue Virus (DENV) | NS3, NS4B and/or NS5, including | |
| | segments and combinations thereof | |

LNP formulations can be prepared using the methods described in the Examples above. All lipid concentrations are shown as mol % as a percentage of the total lipid in the LNP. In some embodiments, compositions can vary in conjugated lipid content from 0.5-5 mol %, in sterol content from 25-45 mol %, in ICL content from 40-65 mol %, in anionic phospholipid content from 1.0-10 mol %, and in total noncationic phospholipid content from 2.5-15 mol %. The lipid nanoparticle (LNP) composition comprises a mRNA nucleic acid encapsulated in lipidic nanoparticles formed from the ionizable lipid disclosed herein at a N/P ratio of 3.5 to 8 relative to the mRNA nucleic acid. For example, compositions can vary in conjugated lipid content from 1.5 to 4 mol %, in sterol content from 17.5-45.5 mol %, in ICL content from 48-56 mol %, in saturated phosphatidyl-L-serine or phosphatidylglycerol content from 2.5-7.5 mol %, in N/P ratio from 4 to 7, and in total noncationic phospholipid content from 2.5-15 mol %.

In some embodiments, the LNP compositions can include 46-56 mol % ICL, (10-X) mol % phospholipid, X mol % anionic phospholipid (wherein X is 0.1-10), 1.5-4 mol % conjugated lipid, and with the rest being cholesterol. In some embodiments, the LNP compositions can include 48-56 mol % ICL, (10-X) mol % phospholipid, X mol % anionic phospholipid (wherein X is 2.5-7.5), 1.5-4 mol % conjugated lipid, and cholesterol. In some embodiments, the LNP compositions can include 48-56 mol % ICL, (10-X) mol % phospholipid, X mol % phosphatidyl-L-serine anionic phospholipid (wherein X is 2.5-7.5), 1.5-4 mol % conjugated lipid, and cholesterol.

In some embodiments, the LNP comprises an anionic phospholipid for targeting the LNP uptake to dendritic cells. In some embodiments, the anionic phospholipid is a phosphatidyl-L-serine or phosphatidylglycerol. Most compositions would contain two phospholipids, typically phosphatidyl-L-serine (DSPS or DPPS being preferred) or phosphatidylglycerol (e.g. DSPG) and phosphatidylcholine, although some exemplary formulations may contain more than two phospholipids. LNPs may contain mRNA or other nucleic acid at N/P of 5-7.

In some embodiments, compositions can vary in conjugated lipid content from 1.5 to 4 mol %, in sterol content from 17.5-45.5 mol %, in ICL content from 48-56 mol %, in saturated phosphatidyl-L-serine or phosphatidylglycerol content from 2.5-7.5 mol %, and in total noncationic phospholipid content from 2.5-15 mol %. In some embodiments, compositions can consist of 1.5 mol % conjugated lipid, 48-56 mol % of an ICL of Formula (I), X mol % phosphatidyl-L-serine anionic phospholipid (X=2.5-7.5), (10-X) mol % noncationic phospholipid, and Y mol % cholesterol (wherein Y is the total mol % of the conjugated lipid, the anionic phospholipid, and the noncationic phospholipid).

### Example 4. LNP Characterization

A. mRNA Encapsulation Efficiency

Materials: Quant-iT™ RiboGreen™ RNA reagent (1 mL solution in DMSO), 20×TE (25 mL in DEPC-treated water), DNase/RNase-Free Distilled Water (Invitrogen Cat #10977-023, or equivalent), 96-Well Clear-Bottom Microplate MED BINDING (Greiner Bio-One Cat #655096), Detergent (Triton™): Surfact-Amps™ X-100 Cat #28314, RNase/DNase-free 1.5 mL Microcentrifuge tubes (ThermoFisher, Invitrogen Cat #AM12400) or equivalent sterile, nuclease-free tubes. A portion of 1×TE buffer prepared for the experiment was allotted for the dilution of RiboGreen dye. This dye was diluted 200× in 1×TE buffer. As the dye is light sensitive, allow to come to RT protected from light. Ensure the dye has melted (become liquid) as it will remain crystalline in temperatures below RT. Before pipetting the dye vortex mix for one second, three times. Prepare the volume of 1×TE buffer needed in a 15 mL conical tube. Vortex or invert the diluted dye and store protected from light. Standards corresponding to 2500, 2000, 1500, 1200, 1000, 800, 500, 250, 50, 25 and 0 ng/mL mRNA were prepared in 1×TE buffer from the mRNA used in the preparation of LNPs. The blank (0 ng/mL) contains no mRNA. LNPs are diluted in 1×TE buffer to approximately 2 μg/mL. 100 μL of blank, standards and samples were added to the wells of a 96-well plate in triplicate to which 100 μL of diluted Ribogreen is added and mixed well by pipetting up and down 4-5 times. The plate is covered with foil to protect from light and after 5 min incubation at room temperature. The standards and samples were read using a microplate fluorometer (e.g., SpectraMax M2) using excitation at 485 nm, emission at 530 nm, and instrument default acquisition parameters. A 2% Triton solution is prepared from the 10% stock solution of Surfact-Amps™ X-100 by performing a 1:5 dilution in 1×TE buffer. Invert 5× to mix. Once the pre-Triton readings have been taken and saved, empty prepared Triton (diluted to 2% in 1×TE buffer) into a clean reagent reservoir. A multichannel pipettor was used to add 20 μL of 2% Triton to each plate well and slowly pipette mix 5×. The plate can be read 10 minutes after mixing. The pre-Triton mRNA concentrations are determined by extrapolation to the pre-Triton standard curves and the post-Triton mRNA concentrations are determined by extrapolation to the Triton containing standard using a linear curve fit.

% Encapsulation Efficiency (% E.E) expresses the concentration of encapsulated mRNA as a percent of the total, post-Triton concentration. The formula can be expressed as:

$$\% Encapsulation Efficiency = 100 - ( ( Pre - Triton concentration / Post - Triton concentration ) ⋆ 100 )$$

### B. LNP Particle Size

30 μL of LNP was mixed with 1.5 mL of 0.1 μm filtered PBS in a polystyrene cuvette (Sarstedt, #67.754) and analyzed for particle size using a ZetaSizer Pro (Malvern) The Z-average size and polydispersity index value were obtained by the method of cumulants using ZS Xplorer software, version number 1.4.0.105. Typically, size measurements of LNPs were taken post LNP mixing, post buffer exchange, post sterile filtering and after freezing for 30 min at −80C and thawing at 37° C.

### C. LNP Zeta Potential

30 μL of LNP is mixed with 1.5 mL 10 mM 2-(N-morpholino)ethanesulfonic acid (MES), 15 mM sodium chloride, pH 5 or 10 mM 4-(2-hydroxyethyl)-1-piperazineethanesulfonic acid (HEPES), 15 mM sodium chloride, pH 7 and injected into a disposable folded capillary cell (Malvern Nanoseries DTS1070) and zeta potential measured on a ZetaSizer Pro at 25° C.

### D. LNP Apparent pKa Determination

1.0 L of buffer consisting of 10 mM sodium phosphate, 10 mM ammonium acetate, 10 mM sodium citrate, and 150 mM sodium chloride solution in WFI was prepared. The solution is divided into 50 mL aliquots and the pH of each adjusted with 5M HCl or 10N NaOH to span the pH range of 2-12 in approximately 0.5-unit increments. The actual pH of each aliquot was measured with a pH meter (Thermo Orion 4 Star with a glass combination micro pH electrode (8220BNWP) calibrated with Thermo Orion pH 4.01, 7.00, 10.01 standards).

A 10 mM solution of 6-p-Toluidino-2-naphthalenesulfonic acid (TNS, Abcam ab275049) in 10 mL of DMSO is prepared from which a 1 mM solution in water is prepared. LNPs were diluted to 1 μg/mL mRNA in a well of a 96-well plate in a final volume of 200 uL for each pH prepared above and TNS added to a final concentration of 10 μM after which each well is mixed by pipetting up and down 10 times with a multichannel pipette. The plate was incubated at room temperature for 3 min, then read on a plate reader Ex.331/Em.445 nm.

A plot of pH versus TNS fluorescence signal was prepared in Graph Pad Prism. The pH value at the half maximum value of fluorescence represents the pKa of nanoparticles and is calculated by performing a non-linear regression analysis for a half maximal fluorescence. This value is the pH at which the numbers of ionized (protonated) and deionized groups are equal.

### Example 5: In Vitro Assessment of Biodegradability of Ionizable Cationic Lipids Using Pancreatin Enzymes

FIG. 9A and FIG. 9B are graphs of the biodegradability of ionizable cationic lipids (ICLs) measured after 15 minutes (FIG. 9A) and 3 hours (FIG. 9B) in the presence of pancreatin enzymes (including trypsin, amylase and lipase) measured according to the assay of Example 5. FIG. 9B is a graph of the biodegradability of ionizable cationic lipids (ICLs) measured after 3 hours in the presence of pancreatin enzymes (including trypsin, amylase and lipase) measured according to the assay of Example 5 (below). All of the ionizable cationic lipids of Formula (I) showed significantly greater degradation compared to the ALC-0315, SM-102 or KC3-OA ionizable lipid comparator compounds.

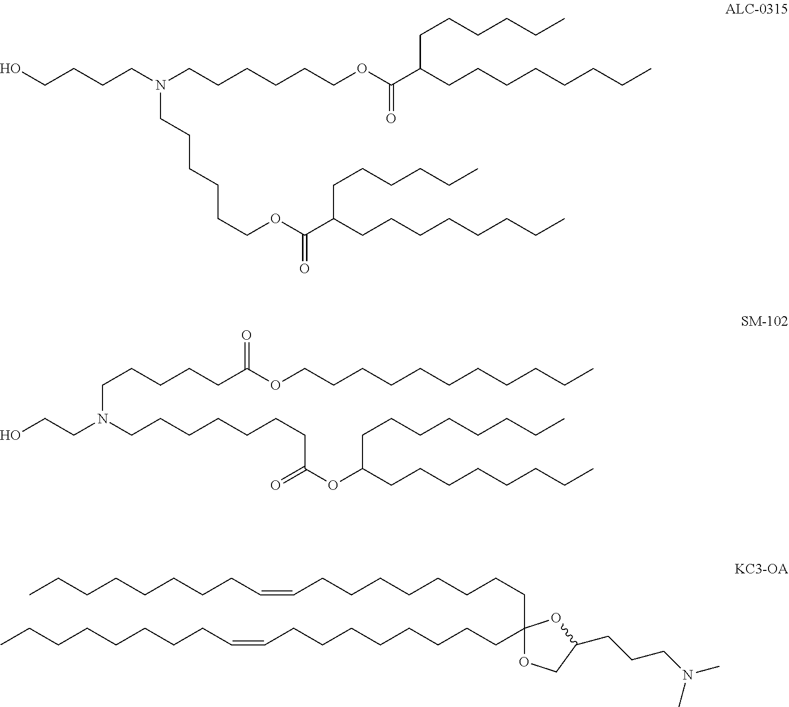

TABLE 5A

| ICL | t = 15 min. | t = 3 hr |
|---|---|---|
| KC3-OA | 1.00 | 1.02 |
| ALC-0315 | 0.92 | 0.94 |
| SM-102 | 0.89 | 0.53 |
| KC3-X1-PH5 | 0.65 | 0.19 |
| KC3-X1-S | 0.50 | 0.16 |
| KC3-X1-Z | 0.02 | <0.01 |
| KC3-X1-PH6 | 0.36 | 0.22 |
| KC3-X2-K9 | 0.34 | 0.01 |
| KC3-X1-AA | 0.23 | 0.01 |
| KC3-X1-PH8 | 0.13 | 0.06 |
| KC3-X1-PH12 | 0.29 | 0.03 |

FIG. 9C is a graph showing the % biodegradation of ALC-0315, SM-102, KC3-X1-AA and KC3-X1-Z measured every half hour over 3 hours. Notably, the KC3-X1-AA and KC3-X1-Z ionizable lipids degraded significantly faster and to a greater extent than either ALC-0315 or SM-102.

An in vitro assay was developed to evaluate the biodegradability of ionizable cationic lipids (ICLs) in the presence of pancreatin enzymes (including trypsin, amylase and lipase).

Individual ICL stock solutions were prepared in ethanol at a concentration of 50 mg/mL and stored at −80° C. until use. Bile salts (Millipore, Cat. No. 48305) were dissolved in Dulbecco's phosphate-buffered saline (DPBS; Sigma, Cat. No. 59300C) at 2 mg/mL, and pancreatin (Sigma, Cat. No. P7545) was prepared at 10 mg/mL in the same buffer. The pH of the DPBS was adjusted to 8.0 using 1 M NaOH. All solutions were maintained at 37° C. under continuous mixing.

For each reaction, 50 nmol of ICL (from the 50 mg/mL ethanol stock) was added to 2 mL of pancreatin solution. Reactions were performed in duplicate across six time points.

The mixture was vortexed for 30 seconds, after which 100 μL aliquots were transferred into separate 2 mL tubes (two replicates per time point). Samples were incubated at 37° C. with shaking.

To terminate the reaction, 10 volumes of isopropanol/acetonitrile (IPA/MeCN, 70:30 v/v) were added. The resulting mixture was sonicated for 5 minutes to facilitate phase separation of lipids and enzymes, followed by centrifugation at 14,000×g for 15 minutes. The supermatant was collected and transferred to TIPLC vials for analysis.

Lipid degradation was quantified using a Thermo Scientific Vanquish Flex UHPLC system equipped with a Charged Aerosol Detector (CAD) and a C₈ column (Phenomenex Kinetex 1.7 m C18 100 Å 150×2.1 mm). Operating conditions for UPLC are detailed in Table 5B.

The extent of lipid degradation was expressed as the percentage of the main ICL peak remaining at each time point relative to the peak area at time zero. Quantification was performed using ICL standards prepared in IPA at concentrations ranging from 5 to 25 g/mL.

TABLE 5B
Chromatographic Conditions

| HPLC Instrument | Thermo Scientific Vanquish Flex UHPLC | | |
|---|---|---|---|
| HPLC Column | C18 column (Phenomenex Kinetex 1.7 μm | | |
| | C18 100 Å 150 × 2.1 mm) | | |
| HPLC guard column | None | | |
| Column Temperature | 50° C. | | |
| Flow Rate, ml/min | 0.35 | | |
| Injection Volume | 5 μL | | |
| CAD | 10H | | |
| Run Time | 12 min | | |
| Sample Solvent | 70% IPA/30% MeCN | | |
| Mobile Phase A | 0.1% TFA in water | | |
| Mobile Phase B | 0.05% TFA in MeCN | | |
| | Time, | Mobile | Mobile |
| | min | Phase A % | Phase B % |
| Gradient | −0.5 | 15 | 85 |
| | 0 | 15 | 85 |
| | 0.5 | 15 | 85 |
| | 5 | 5 | 95 |
| | 11 | 5 | 95 |
| | 11.5 | 15 | 85 |
| | 12 | 15 | 85 |

### Example 6A. Determination of Transfection Efficiency in Murine Dendritic Cells of LNPs Using Firefly Luciferase mRNA

Immortalized mouse dendritic cells (MutuDC 1940, Fuertes Marraco et al., Front.

Immunol. 2012, 3:331) are cultivated in Gibco™ 1IMDM medium with GlutaMax™ (Gibco cat. No 31980097), supplemented with 7.5 mg/ml NaHCO₃, 10 mM HEPES-Na pH 7, 50 M beta-mercaptoethanol, 50 U/ml penicillin, 50 μg/mL streptomycin, and 10% fetal bovine serum, at 37° C., 5% CO₂. The cells are rinsed with phosphate-buffered saline (PBS, Cytiva, cat. No SH30526) harvested using 5 mM EDTA in PBS (PBS-EDTA), resuspended in the growth medium, inoculated in white opaque 96-well cell culture plates (e.g., Costar cat. No 3917) in the wells in columns 3-10 of the plate at 15,000 cells/well in 0.1 ml of the medium, and allowed to acclimate overnight. The media are aspirated, replaced with 0.1 ml of the media containing the desired concentration of mRNA LNP (0.1-1 μg/mL mRNA, typically 0.3 μg/mL mRNA), and incubated at 37° C., 5% CO₂ for the desired transfection period, typically 24 hours. Three to four hours prior to the end of the transfection period, the wells 2A and 11A of the plates are seeded with 0.1 ml of the cell suspension standard containing approximately 1,000,000 viable cells/mL (the viable cell concentration is accurately determined), and wells 2B to 2G, and 11B to 11G are seeded with the serial dilutions of the standard at the dilution factor of 1.5 to generate eight cell quantity standards per plate in duplicate. In the wells 1A-11H, 12A-12H, 0.1 ml of the cell medium is added. At the end of the transfection period, 11 μL of 20 mM aqueous solution of Hoechst 33342 DNA fluorescent dye (Ultrapure grade, cat. No 17533, AAT Bio, USA) is added to each well, carefully mixed by gentle tapping and swirling of the plate by hand, and the plate is incubated at 37° C., 5% CO₂ for one hour to allow for the staining of the cellular DNA by the added dye.

Luciferase standards are prepared using commercial recombinant firefly luciferase (e.g. cat. No. SRE0045, Sigma-Aldrich, USA), reconstituted to 2 mg/ml protein in 1 M Tris-HCl buffer pH 8.5 and stored at −80° C. An interim standard is prepared by diluting the reconstituted luciferase 20-fold (to 0.1 mg/ml) in PBS-EDTA containing 1 mg/ml nuclease- and protease-free bovine serum albumin (PBS-EDTA-BSA). Eight working standards (DA to DH) are prepared by further diluting the interim standard in PBS-EDTA-BSA, first to 1000 ng/mL (DA), and then serially diluting DA down to DH with the dilution factor of three per step. The interim and working standards are prepared fresh.

At the end of one hour incubation, the media from the wells in columns 2-11 of the plate are aspirated, and 0.1 ml of PBS-EDTA is added. The plate is incubated at 37° C., 5% CO₂ for 10 min and on the microplate shaker at 700 rpm for 3 min to dislodge the cells. Then the media from the wells 1A-1H, 12A-12H are aspirated and 0.1 ml aliquots of the luciferase working standards are added, from the highest (DA) in 1A and 12A, to the lowest (DH) in 1H and 12H, creating a series of eight luciferase standards per plate in duplicate (plate columns 1 and 12).

The Hoechst 33342 fluorescence of the cell suspensions in the wells is determined using SpectraMax M2 multimode plate reader (Molecular Devices, USA) using excitation at 360 nm, emission at 461 nm, and nine-point well scan option. The plate is auto-mixed for 5 sec prior to measurements. The average fluorescence signal per each well is determined, and the fluorescence readings of the cell standard wells 2A-2H, 11A-11H are used to generate the linear standard curve of the average fluorescence vs. cell concentration in the well (0.1 ml). The cell concentration in the 0.1 ml of the cell suspension at the end of transfection is determined from this standard curve for each well in a conventional manner.

The luciferase concentration in the cell suspensions is determined using BrightGlo® Firefly Luciferase assay kit (Promega Corp., USA). To each of the luciferase standard wells, test sample wells, and blank (cells only) wells 2G-2H, 11G-11H in the same plate, 0.1 mL of the reconstituted complete Luciferase substrate buffer (prepared according to the manufacturer's recommendations) is added, the contents of the wells are thoroughly mixed, and the luminescence of the mixtures in the wells is determined at 10-15 min after the luciferase substrate addition using SpectraMax M2 multimode plate reader (Molecular Devices, USA) in the luminescence mode under the instrument default settings. The readings of the duplicate standards in the wells 1A-1H and 12A-12H, after subtraction of the blank readings of the wells 2G-2H and 11G-11H, are used to generate the Luciferase standard curve linearly approximated in double-log coordinates using the minimization of the mean squared relative error. The amount of luciferase formed in each well is calculated from the luminescence readings (with blank readings subtracted) using the luciferase standard curve, normalized to the number of cells per well determined using the Hoechst 3342 method above, and presented in ng of luciferase per 1 million (10⁶) cells. A normalization to the post-transfection cell density of 40,000 cells/well was made according to Example 6B.

SEQ ID NO: 2. is a firefly luciferase protein sequence.

SEQ ID NO: 3 is a codon optimized mRNA sequence encoding firefly luciferase. The open reading frame is flanked on the 5′ end with the HBB 5′ UTR containing an optimized Kozak sequence and the 3′ end with the HBB 3′ UTR.

SEQ ID NO: 4 is a sequence encoding a human hemoglobin subunit beta (HBB) untranslated regions (UTRs). NCBI GenBank reference sequence NM_000518.5. The 5′ UTR is underlined and spans from nucleotides 1-44. The 3′ UTR is underlined and spans from nucleotides 495-628.

### Example 6B. Effect of Cell Density on the Luciferase mRNA Expression in MutuDC-1940 Cells after Targeted LNP Transfection

MutuDC-1940 cells were seeded in 96-well plates as described in Example 6A at various cell plating densities in quadruplicate. The cells were acclimated for 24 hours and transfected with luciferase mRNA (SEQ ID NO: 3 encoding for SEQ ID NO: 2) formulated in the PS-targeted or non-targeted LNP prepared from the ICL KC3-OA Lot NT was formulated using KC3-OA/DSPC/Chol/PEG-DMG (48 mol %/10 mol %/40.5 mol %/1.5 mol %) with an N/P ratio of 5.25. The Z-average size was 116.5 nm and % EE was 89.51±2.94. Lot T1 was formulated using KC3-OA/DSPC/DPPS-NH4/Chol/PEG-DMG (48 mol %/5 mol %/5 mol %/40.5 mol %/1.5 mol %) with an N/P ratio of 5.25. The Z-average size was 106.4 nm PDI 0.06 and % EE 91.8±3.1. Lot T2 was formulated using KC3-OA/DSPC/DPPS-NH4/Chol/PEG-DMG (48 mol %/5 mol %/5 mol %/40.5 mol %/1.5 mol %) with an N/P ratio of 5.25. The Z-average size was 108.7 nm PDI 0.05 and % EE 93.05±1.68. LNPs were diluted into the cell medium at 0.3 μg/ml mRNA, 0.1 ml/well, for 24 hours. The cells were enumerated, and the Luciferase mRNA transfection activity was determined according to Example 6A. The study was repeated twice, using the plating density ranges of (6.5-49.0)×10³ cells/well, and (1.32-22.5)×10³ cells/well, and the data were pooled. The results indicated that Luc expression normalized to the final number of the cells in the well was dependent on both initial (plating) cell density and final (post-transfection) cell density (FIG. 26). It was found that for the targeted LNP and the typical assay range of post-transfection cell concentrations of (99-1251)×10³ cell/ml in 0.1 ml of the cell harvesting solution ((9.9-125.1)×10³ cells/well), observed under the assay conditions of Example 6A, the Luc expression was in good agreement with the second-degree polynomial approximation against the logarithm of the post-transfection cell concentration (R²=0.9925) (FIG. 27). Therefore, for targeted LNPs the assayed luciferase activities in each individual well were corrected to the standard post-transfection cell concentration of 400×10³ cells/ml (cell density 40×10³ cells/well) using the formula: Luc=Luc(D)*100/(91.6×log²(D)−659.2×log(D)+1196.4), where D is the measured post-transfection cell concentration in 10′ cells/ml, Luc(D) is the measured luciferase expression at the post-transfection cell concentration D.

### Example 7. Evaluating the Effect of ICL Mol % and PS Targeting on Activity of LNPs Containing Biodegradable ICLs

LNPs were prepared and characterized as described in Examples 3 and 4, with the mRNA formulated was luciferase mRNA (SEQ ID NO: 3 encoding for SEQ ID NO: 2), and included the ICLs indicated in the data tables below. Unless explicitly indicated otherwise, the isomer form of the phosphatidylserine lipids used in all the Examples is phosphatidyl-L-serine.

Table 7-A lists the chemical structures of compounds tested in Example 7. For compounds of Formula (I), (x) refers to the number of carbon atoms in X₁ and X₂ and (y) refers to the total number of carbon atoms in Y₁ and Y₂.

TABLE 7-A
Additional compounds tested in Example 7.

| Compound | Structure |
|---|---|
| C16-Phyt- DM2 | 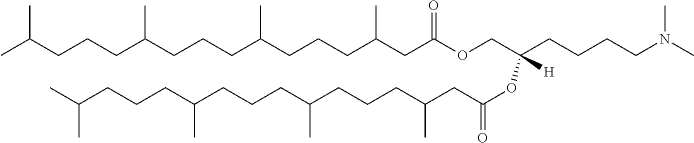 [SMILES: CC(C)CCCC(C)CCCC(C)CCCC(C)CC(=O)OC[C@H](CCCCN(C)C)OC(=O)CC(C)CCCC(C)CCCC(C)CCCC(C)C] |
| C20-DM2 | 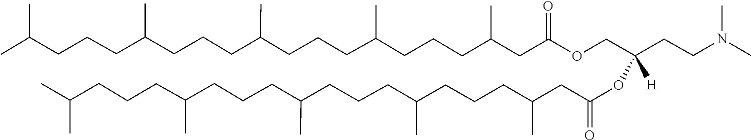 [SMILES: CC(C)CCCC(C)CCCC(C)CCCC(C)CCCC(C)CC(=O)OC[C@H](CCN(C)C)OC(=O)CC(C)CCCC(C)CCCC(C)CCCC(C)CCCC(C)C] |
| C20 (8-9; cis-8- Eicosenoic Acid FA) DM2 | 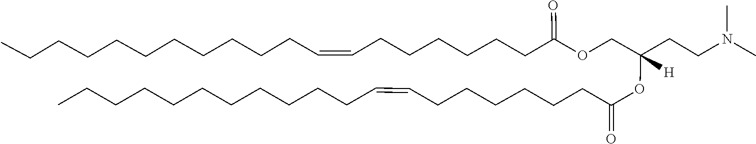 [SMILES: CCCCCCCCCCC/C=C\CCCCCCC(=O)OC[C@H](CCN(C)C)OC(=O)CCCCCC/C=C\CCCCCCCCCCC] |
| C20 (9-10; gadoleic acid) | 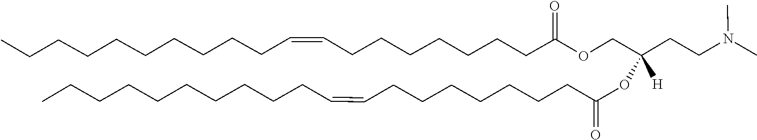 [SMILES: CCCCCCCCCC/C=C\CCCCCCCC(=O)OC[C@H](CCN(C)C)OC(=O)CCCCCCC/C=C\CCCCCCCCCC] |

### Example 7A Evaluating LNPs Comprising KC3-OA, C6-Phyt-DM2, KC3X1-I, KC3X1-H, and KC3-X1-PH CLs, with and without PS-Targeting

PS-Targeted LNPs were prepared with 5 mol % DSPC, 5 mol % DPPS, 1.5 mol % PEG-DMG, and 48 mol %, 50 mol % or 52 mol % ICL with 40.5 mol %, 38.5 mol % or 36.5 mol T of cholesterol respectively, and with an N/P of 5.25. A non-targeted LNP formulation of each ICL was prepared with 48 mol % ICL, 10 mol % DSPC, 40.5 mol cholesterol and 1.5 mol % PEG-DMG, with an N/P of 5.25.

Particle size refers to the Z-Average particle size. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 7A-1
LNP size characterization of DPPS-targeted LNPs made with
varying ICLs and non-targeted (NT) LNPs. Table 7A-2. Particle
size refers to the Z-Average particle size. Post-F/T refers
to samples that underwent a freeze-thaw cycle to evaluate
their stability (20 min at −80° C., thawing at 37°
C.). NT refers to LNP formulation that are not targeted with PS.

| | | Post Filter | | Post F/T | |
|---|---|---|---|---|---|
| | ICL mol % | Z-Average | | Z-Average | |
| (Experiment) ICL | Composition | (nm) | PDI | (nm) | PDI |
| KC3-OA, NT | 48 | 112.4 | 0.100 | 112.3 | 0.064 |
| KC3-OA | 48 | 113.8 | 0.113 | 114.8 | 0.114 |
| KC3-OA | 50 | 115.7 | 0.065 | 113.6 | 0.099 |
| KC3-OA | 52 | 109.7 | 0.064 | 107.1 | 0.118 |
| C16 Phyt-DM2, NT | 48 | 94.1 | 0.107 | 97.4 | 0.161 |
| C16 Phyt-DM2 | 48 | 85.9 | 0.134 | 85.6 | 0.214 |
| C16 Phyt-DM2 | 50 | 83.9 | 0.110 | 84.5 | 0.105 |
| C16 Phyt-DM2 | 52 | 87.1 | 0.090 | 87.7 | 0.097 |
| KC3-X1-I, NT | 48 | 106 | 0.149 | 118.8 | 0.198 |
| KC3-X1-I | 48 | 118.2 | 0.141 | 119.6 | 0.156 |
| KC3-X1-I | 50 | 118 | 0.134 | 119.3 | 0.151 |
| KC3-X1-I | 52 | 121.4 | 0.183 | 124.0 | 0.167 |
| KC3-X1-H, NT | 48 | 111.9 | 0.087 | 142.9 | 0.154 |
| KC3-X1-H | 48 | 124.0 | 0.153 | 125.3 | 0.193 |
| KC3-X1-H | 50 | 125.6 | 0.134 | 126.2 | 0.125 |
| KC3-X1-H | 52 | 130.2 | 0.129 | 134.4 | 0.145 |
| KC3-X1-PH1, NT | 48 | 104.1 | 0.109 | 106.6 | 0.137 |
| KC3-X1-PH1 | 48 | 112.5 | 0.150 | 114.4 | 0.130 |
| KC3-X1-PH1 | 50 | 111.9 | 0.125 | 111.9 | 0.127 |
| KC3-X1-PH1 | 52 | 114.6 | 0.119 | 118.5 | 0.138 |

TABLE 7A-2
Zeta refers to zeta potential measurements
at either pH 5 or pH 7 in units of mV.

| | mol % | zeta | zeta |
|---|---|---|---|
| ICL | Composition | pH 5 | pH 7 |
| KC3-OA, NT | 48 | 22.7 | 2.4 |
| KC3-OA | 48 | 21.7 | 1.1 |
| KC3-OA | 50 | 20.8 | 0.4 |
| KC3-OA | 52 | 21.8 | −0.4 |
| AKC-C16 Phyt-DM2, NT | 48 | 18.0 | −6.5 |
| AKC-C16 Phyt-DM2 | 48 | 19.9 | −10.9 |
| AKC-C16 Phyt-DM2 | 50 | 20.1 | −11.6 |
| AKC-C16 Phyt-DM2 | 52 | 20.7 | −11.5 |
| KC3-X1-I, NT | 48 | 22.2 | 0.2 |
| KC3-X1-I | 48 | 20.0 | −4.9 |
| KC3-X1-I | 50 | 20.5 | −4.6 |
| KC3-X1-I | 52 | 21.8 | −4.6 |
| KC3-X1-H, NT | 48 | 20.8 | 0.6 |
| KC3-X1-H | 48 | 19.6 | −6.0 |
| KC3-X1-H | 50 | 23.8 | −5.1 |
| KC3-X1-H | 52 | 19.1 | −5.9 |
| KC3-X1-PH1, NT | 48 | 25.8 | 6.8 |
| KC3-X1-PH1 | 48 | 24.7 | 1.6 |
| KC3-X1-PH1 | 50 | 22.8 | 0.8 |
| KC3-X1-PH1 | 52 | 23.7 | 0.8 |

TABLE 7A-3
LNP mRNA encapsulation efficiency (E.E, %) of DPPS-targeted
LNPs made with varying ICLs and non-targeted (NT) LNPs.

| | mol % | % E.E | % E.E |
|---|---|---|---|
| ICL | Composition | filtered | F/T |
| KC3-OA, NT | 48 | 82.4 ± 3.0 | 84.6 ± 4.1 |
| KC3-OA | 48 | 81.3 ± 1.2 | 84.4 ± 1.2 |
| KC3-OA | 50 | 80.8 ± 3.5 | 84.2 ± 2.9 |
| KC3-OA | 52 | 85.2 ± 4.3 | 86.8 ± 2.3 |
| AKC-C16 Phyt-DM2, NT | 48 | 76.7 ± 3.3 | 79.3 ± 1.4 |
| AKC-C16 Phyt-DM2 | 48 | 80.1 ± 3.3 | 79.6 ± 3.2 |
| AKC-C16 Phyt-DM2 | 50 | 77.7 ± 1.7 | 77.6 ± 3.1 |
| AKC-C16 Phyt-DM2 | 52 | 79.5 ± 3.5 | 78.1 ± 1.2 |
| KC3-X1-I, NT | 48 | 91.8 ± 5.5 | 87.5 ± 3.3 |
| KC3-X1-I | 48 | 87.6 ± 2.7 | 87.8 ± 3.9 |
| KC3-X1-I | 50 | 89.4 ± 2.8 | 89.0 ± 3.9 |
| KC3-X1-I | 52 | 88.7 ± 3.0 | 89.0 ± 3.0 |
| KC3-X1-H, NT | 48 | 86.0 ± 2.3 | 75.1 ± 2.6 |
| KC3-X1-H | 48 | 83.5 ± 5.1 | 84.6 ± 2.5 |
| KC3-X1-H | 50 | 84.5 ± 1.5 | 85.0 ± 2.2 |
| KC3-X1-H | 52 | 83.2 ± 4.4 | 83.5 ± 2.8 |
| KC3-X1-PH1, NT | 48 | 95.2 ± 3.5 | 94.8 ± 6.5 |
| KC3-X1-PH1 | 48 | 95.7 ± 2.9 | 95.9 ± 5.2 |
| KC3-X1-PH1 | 50 | 95.4 ± 4.7 | 95.5 ± 5.3 |
| KC3-X1-PH1 | 52 | 95.2 ± 2.7 | 95.3 ± 6.6 |

The size range of the LNPs described in Table 7A-1 ranged from 83.9 nm to 130.2 nm. Upon freezing and thawing, the samples changed in size between 0-3.3% for the PS-targeted samples and 0.1-21.7% for the non-PS containing samples. The mRNA encapsulation efficiency was between 76-950.7% for all samples. Upon freezing and thawing, the samples changed in % E.E between 0.1-30.7% for the PS-targeted samples and 0.4-10.8% for the non-PS containing samples.

From this data we observed that 5 mol % PS has a stabilizing effect on the LNP sizes and % E.E making LNPs less susceptible to size increase and mRNA release or exposure to the external media during the freeze/thaw event.

TABLE 7A-4
LNP mRNA Luciferase mRNA transfection activity of DPPS-targeted
LNPs made with varying ICLs at 48, 50, & 52 mol %. and non-
targeted (NT) LNPs made at 48 mol %. Samples were thawed from
−80° C. storage, diluted to 0.3 ug/mL mRNA and added
to MutuDC1940 cells as described in Example 6A. The ratio
of luc expression for the targeted PS-containing LNPs at each
mol % divided by the luc expression of the non-targeted non-PS
containing LNPs at 48 mol % ICL is displayed as T/NT.

| | mol % | Luc | |
|---|---|---|---|
| ICL | Composition | ng/10\hat{ }6 cells | T/NT |
| KC3-OA, NT | 48 | 51.4 ± 4.6 | 1.0 |
| KC3-OA | 48 | 265.9 ± 20.9 | 5.2 |
| KC3-OA | 50 | 284.3 ± 38.2 | 5.5 |
| KC3-OA | 52 | 305.2 ± 40.9 | 5.9 |
| AKC-C16 Phyt-DM2, NT | 48 | 29.2 ± 2.3 | 1.0 |
| AKC-C16 Phyt-DM2 | 48 | 112.9 ± 12.0 | 3.9 |
| AKC-C16 Phyt-DM2 | 50 | 113.6 ± 9.4 | 3.9 |
| AKC-C16 Phyt-DM2 | 52 | 117.7 ± 8.2 | 4.0 |
| KC3-X1-I, NT | 48 | 23.1 ± 5.4 | 1.0 |
| KC3-X1-I | 48 | 226.4 ± 31.0 | 9.8 |
| KC3-X1-I | 50 | 221.6 ± 27.2 | 9.6 |
| KC3-X1-I | 52 | 239.4 ± 25.4 | 10.3 |
| KC3-X1-H, NT | 48 | 14.0 ± 2.3 | 1.0 |
| KC3-X1-H | 48 | 335.6 ± 30.0 | 23.9 |
| KC3-X1-H | 50 | 373.8 ± 34.6 | 26.6 |
| KC3-X1-H | 52 | 429.8 ± 28.5 | 30.6 |
| KC3-X1-PH1, NT | 48 | 10.0 ± 2.2 | 1.0 |
| KC3-X1-PH1 | 48 | 349.5 ± 33.8 | 35.0 |
| KC3-X1-PH1 | 50 | 330.3 ± 21.1 | 33.1 |
| KC3-X1-PH1 | 52 | 364.4 ± 21.6 | 36.5 |

In general, we observed an increase in luc expression with increasing amounts of ICL within each set of formulations. The luc expression range was 112.9-429.8 for the PS-targeted samples and 10-51.4 for the non-PS containing LNPs. On average over the 3 ICL mol % compositions tested for each ICL, the rank order of the ICL activity was found to be KC3-X1-H, KC3-X1-PH1, KC3-OA, KC3-X1-I, and AKG-C16 Phyt-DM2. The rank order of the T/NT values at 48 mol % from highest to lowest were found to be KC3-X1-PH1, KC3-X1-H, KC3-X1-I, KC3-OA and AKG-C16 Phyt-DM2.

### Example 7B Evaluating LNPs Comprising KC2-X1-G, KC3-X1-J, KC3-X1-PH4, KC3-X1-PH4-B, KC3-X1-F, and KC3-X1-G ICLs, with and without PS-Targeting

PS-Targeted LNPs were prepared with 5 mol % DSPC, 5 mol % DPPS, 1.5 mol % PEG-DMG, and 48 mol %, 50 mol % or 52 mol % ICL with 40.5 mol %, 38.5 mol % or 36.5 mol % of cholesterol respectively, and with an N/P of 5.25. A non-targeted LNP formulation of each ICL was prepared with 48 mol % ICL, 10 mol % DSPC, 40.5 mol % cholesterol and 1.5 mol % PEG-DMG, with an N/P of 5.25.

Particle size refers to the Z-Average particle size. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 7B-1
Particle size refers to the Z-Average particle size. Post-F/T refers to samples that
underwent a freeze-thaw cycle to evaluate their stability (20 min at −80°
C., thawing at 37° C.). NT refers to LNP formulation that are not targeted with PS.

| | | Post | | Post | |
|---|---|---|---|---|---|
| | | Filter | | F/T | |
| | | Z- | | Z- | |
| | | Average | | Average | |
| ICL | Composition | (nm) | PDI | (nm) | PDI |
| AKG-KC2-X1-G, NT | 48 | 112.2 | 0.0705 | 114.7 | 0.18 |
| AKG-KC2-X1-G | 48 | 131.1 | 0.1725 | 137.1 | 0.1265 |
| AKG-KC2-X1-G | 50 | 129.9 | 0.152 | 139.5 | 0.1181 |
| AKG-KC2-X1-G | 52 | 132.9 | 0.0948 | 139.4 | 0.1347 |
| AKG-KC3-X1-J, NT | 48 | 102.1 | 0.0913 | 109.1 | 0.2511 |
| AKG-KC3-X1-J | 48 | 99.9 | 0.1586 | 104.6 | 0.1556 |
| AKG-KC3-X1-J | 50 | 105.8 | 0.1332 | 117.2 | 0.1261 |
| AKG-KC3-X1-J | 52 | 102.3 | 0.1119 | 109.3 | 0.1431 |
| AKG-KC3-X1-PH4, NT | 48 | 121.9 | 0.0684 | 124.5 | 0.1809 |
| AKG-KC3-X1-PH4 | 48 | 127.2 | 0.0973 | 129.2 | 0.06571 |
| AKG-KC3-X1-PH4 | 50 | 131.1 | 0.0836 | 132.1 | 0.1008 |
| AKG-KC3-X1-PH4 | 52 | 143.4 | 0.0759 | 145.3 | 0.07466 |
| AKG-KC3-X1-PH4-B, NT | 48 | 108.1 | 0.0869 | 161.3 | 0.375 |
| AKG-KC3-X1-PH4-B | 48 | 128.1 | 0.105 | 122.3 | 0.1571 |
| AKG-KC3-X1-PH4-B | 50 | 126 | 0.11 | 127.5 | 0.2624 |
| AKG-KC3-X1-PH4-B | 52 | 136.4 | 0.0766 | 137.3 | 0.135 |
| AKG-KC3-X1-F, NT | 48 | 111.6 | 0.1674 | 150.8 | 0.3205 |
| AKG-KC3-X1-F | 48 | 122.6 | 0.1218 | 125.6 | 0.1534 |
| AKG-KC3-X1-F | 50 | 125.1 | 0.1367 | 129.9 | 0.1478 |
| AKG-KC3-X1-F | 52 | 125.6 | 0.1472 | 125.8 | 0.1245 |
| AKG-KC3-X1-G, NT | 48 | 111.4 | 0.1323 | 118.9 | 0.1253 |
| AKG-KC3-X1-G | 48 | 108.7 | 0.122 | 123.9 | 0.1424 |
| AKG-KC3-X1-G | 50 | 111.7 | 0.1141 | 126.2 | 0.1251 |
| AKG-KC3-X1-G | 52 | 104.9 | 0.1295 | 113.2 | 0.1531 |

TABLE 7B-2
Zeta refers to zeta potential measurements at either pH 5
or pH 7 in units of mV. mRNA encapsulation efficiency is
referred to as (E.E, %). F/T refers to a freeze/thaw cycle.

| | | % E.E | % E.E | | |
|---|---|---|---|---|---|
| | | Filtered ± | F/T ± | zeta | zeta |
| ICL | Composition | stdev | stdev | pH 5 | PH 7 |
| AKG-KC2-X1-G, NT | 48 | 89.3 ± 4.1 | 82.6 ± 3.1 | 22.48 | 5.441 |
| AKG-KC2-X1-G | 48 | 47.8 ± 0.8 | 44.5 ± 1.0 | 26.38 | −0.5115 |
| AKG-KC2-X1-G | 50 | 48.7 ± 1.1 | 47.8 ± 2.0 | 24.25 | −0.1725 |
| AKG-KC2-X1-G | 52 | 49.1 ± 1.2 | 48.2 ± 2.4 | 23.07 | −1.34 |
| AKG-KC3-X1-J, NT | 48 | 89.4 ± 3.6 | 89.9 ± 3.4 | 29.02 | 6.938 |
| AKG-KC3-X1-J | 48 | 91.8 ± 2.9 | 92.3 ± 4.1 | 24.17 | 2.617 |
| AKG-KC3-X1-J | 50 | 91.0 ± 2.7 | 90.9 ± 4.7 | 12.39 | 1.881 |
| AKG-KC3-X1-J | 52 | 92.0 ± 2.6 | 92.1 ± 4.0 | 23.64 | 1.891 |
| AKG-KC3-X1-PH4, NT | 48 | 96.8 ± 6.1 | 97.4 ± 5.3 | 24.8 | 5.944 |
| AKG-KC3-X1-PH4 | 48 | 97.4 ± 2.9 | 98.0 ± 9.9 | 24.91 | 0.6742 |
| AKG-KC3-X1-PH4 | 50 | 97.4 ± 4.4 | 97.8 ± 5.5 | 25.53 | 1.472 |
| AKG-KC3-X1-PH4 | 52 | 97.4 ± 5.4 | 97.8 ± 4.6 | 28.05 | 1.312 |
| AKG-KC3-X1-PH4-B, NT | 48 | 95.4 ± 4.0 | 94.4 ± 5.8 | 27.13 | 0.7808 |
| AKG-KC3-X1-PH4-B | 48 | 96.0 ± 3.7 | 96.7 ± 6.0 | 23.14 | −2.736 |
| AKG-KC3-X1-PH4-B | 50 | 96.2 ± 4.6 | 96.8 ± 6.7 | 22.6 | −3.006 |
| AKG-KC3-X1-PH4-B | 52 | 95.5 ± 7.2 | 96.4 ± 4.7 | 25.83 | −3.387 |
| AKG-KC3-X1-F, NT | 48 | 90.1 ± 3.6 | 90.2 ± 2.8 | 25.53 | 2.782 |
| AKG-KC3-X1-F | 48 | 91.4 ± 5.6 | 92.4 ± 5.8 | 22.43 | −0.8576 |
| AKG-KC3-X1-F | 50 | 90.3 ± 3.7 | 91.5 ± 4.2 | 22.2 | −0.8566 |
| AKG-KC3-X1-F | 52 | 91.3 ± 4.0 | 92.8 ± 6.1 | 23.79 | −1.032 |
| AKG-KC3-X1-G, NT | 48 | 89.7 ± 5.3 | 87.6 ± 3.4 | 19.68 | 5.331 |
| AKG-KC3-X1-G | 48 | 91.2 ± 2.7 | 89.0 ± 2.0 | 25.72 | 3.894 |
| AKG-KC3-X1-G | 50 | 90.9 ± 4.5 | 88.3 ± 3.0 | 21.16 | 5.294 |
| AKG-KC3-X1-G | 52 | 96.8 ± 8.6 | 92.3 ± 4.7 | 24.6 | 3.634 |

The size range of the LNPs described in Table 7B-1 ranged from 99.9-143.4 nm. Upon freezing and thawing, the samples changed in size between 0.15-12.3% for the PS-targeted samples and 2.1-3.9 for the non-PS containing samples. The mRNA encapsulation efficiency was between 47.7-97.5% for all samples. Upon freezing and thawing, the samples changed in % E.E between 0.1-4.5% for the PS-targeted samples and 0.1-6.7% for the non-PS containing samples. From this data we observed that 5 mol % PS has a stabilizing effect on the LNP sizes and % E.E making LNPs less susceptible to size increase and mRNA release or exposure to the external media during the freeze/thaw event. The zeta potential range at pH 5 was 12.4-29.0 mV and at pH 7 was −3.4-6.9 mV indicating that the LNPs were positively charged at pH 5 and either slightly negative or slightly positively charged at pH 7. Transfection efficiency was determined in murine dendritic cells of LNPs using a firefly luciferase assay as described in Example 6A.

TABLE 7B-3
LNP mRNA Luciferase mRNA transfection activity of DPPS-
targeted LNPs made with varying ICLs at 48, 50, & 52
mol %. and non-targeted (NT) LNPs made at 48 mol %.

| | | Average (ng/10\hat{ }6 | |
|---|---|---|---|
| Description | Composition | cells) | T/NT |
| AKG-KC2-X1-G, NT | 48 | 1.36 ± 0.32 | |
| AKG-KC2-X1-G | 48 | 115.17 ± 15.3 | 84.90 |
| AKG-KC2-X1-G | 50 | 132.50 ± 25.0 | 97.67 |
| AKG-KC2-X1-G | 52 | 140.94 ± 22.5 | 103.89 |
| AKG-KC3-X1-J, NT | 48 | 0.99 ± 0.2 | |
| AKG-KC3-X1-J | 48 | 61.51 ± 15.2 | 45.35 |
| AKG-KC3-X1-J | 50 | 97.99 ± 30.2 | 72.23 |
| AKG-KC3-X1-J | 52 | 95.41 ± 15.0 | 70.33 |
| AKG-KC3-X1-PH4, NT | 48 | 8.24 ± 1.6 | |
| AKG-KC3-X1-PH4 | 48 | 109.63 ± 20.4 | 80.81 |
| AKG-KC3-X1-PH4 | 50 | 126.39 ± 33.0 | 93.17 |
| AKG-KC3-X1-PH4 | 52 | 156.1 ± 20.7 | 115.07 |
| AKG-KC3-X1-PH4-B, NT | 48 | 20.9 ± 4.8 | |
| AKG-KC3-X1-PH4-B | 48 | 76.1 ± 9.2 | 56.13 |
| AKG-KC3-X1-PH4-B | 50 | 72.7 ± 17.4 | 53.55 |
| AKG-KC3-X1-PH4-B | 52 | 89.2 ± 23.6 | 65.78 |
| AKG-KC3-X1-F, NT | 48 | 4.0 ± 0.9 | |
| AKG-KC3-X1-F | 48 | 79.8 ± 18.4 | 58.81 |
| AKG-KC3-X1-F | 50 | 97.4 ± 20.9 | 71.77 |
| AKG-KC3-X1-F | 52 | 109.7 ± 31.0 | 80.88 |
| AKG-KC3-X1-G, NT | 48 | 2.1 ± 0.4 | |
| AKG-KC3-X1-G | 48 | 66.6 ± 10.4 | 49.08 |
| AKG-KC3-X1-G | 50 | 74.4 ± 16.5 | 54.84 |
| AKG-KC3-X1-G | 52 | 113.6 ± 28.8 | 83.74 |
| 012925-2 Control | | 177.6 ± 22.1 | |

In general, we observed an increase in luc expression with increasing amounts of ICL within each set of formulations The luc expression range was 61.5-156.1 ng/million cells for the PS-targeted samples and 1-20.9 for the non-PS containing LNPs. The T/NT ratio at 48 mol % for these set of lipids ranged from 45.4-84.9. The T/NT ratio increased with increasing ICL mol % in each series when compared against the 48 mol % non-targeted LNP. The control sample was KC3-OA/PS study with 48 mol % ICL from Table 7A-1 that was frozen down into multiple aliquots and freshly thawed for this study.

### Example 7C Evaluating LNPs Comprising KC3-OA, C20 (13-14; Paullinic acid) DM2, KC2-X1-PH5. KC3-X1-K. KC3-X1-L. KC3-X1-M, KC3-X1-PH5, and KC3-X1-PH5B ICLs, with and without PS-Targeting

LNPs were prepared and characterized as described in Examples 3 and 4, except the mRNA formulated was luciferase mRNA (SEQ ID NO: 3 encoding for SEQ ID NO: 2), and included the ICLs: KC3-OA, C20 (13-14; Paullinic acid) DM2, KC2-X1-PH5, KC3-X1-K, KC3-X1-L, KC3-X1-M, KC3-X1-PH5, and KC3-X1-PH5B. PS-Targeted LNPs were prepared with 5 mol % DSPC, 5 mol % DPPS, 1.5 mol % PEG-DMG, and 52 mol % ICL with 36.5 mol % of cholesterol and with an N/P of 5.25. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

Particle size refers to the Z-Average particle size after freezing and thawing. Zeta refers to zeta potential measurements at either pH 5 or pH 7 in units of mV. mRNA encapsulation efficiency is referred to as (E.E,%). The C₂₀ (13-14; Paullinic acid) DM2 ICL based LNP became aggregated during buffer exchange and was not processed or analyzed further.

TABLE 7C-1
Physicochemical properties and characterization
of DPPS containing LNPs containing FFluc mRNA.

| | Particle | | | |
|---|---|---|---|---|
| Lipid | Size | E.E, %, | Zeta | Zeta |
| Composition | (nm) | Stdev | pH 5 | pH 7 |
| KC3-OA | 112.3 | 84.65 | 21.24 | 2.05 |
| C20-DM2 | N/A | N/A | N/A | N/A |
| KC2-X1-PH5 | 102 | 81.25 | 23.35 | −6.25 |
| KC3-X1-K | 117.8 | 83.16 | 29.17 | −0.12 |
| KC3-X1-L | 112 | 82.44 | 23.68 | −0.02 |
| KC3-X1-M | 109.2 | 88.00 | 30.20 | 1.07 |
| KC3-X1-PH5 | 109 | 94.48 | 28.05 | −2.58 |
| KC3-X1-PH5B | 120.5 | 84.13 | 26.28 | −7.08 |

The formulations ranged in size from approximately 102 to 121 nm in diameter. The % entrapped mRNA ranged from 82-95% and LNPs exhibited positive surface charge at pH 5 (~+21 to +30 mV) and a negative to close to neutral charge at pH 7 (−7 to +2 mV).

TABLE 7C-2
Physicochemical properties and characterization of
non-DPPS containing LNPs containing FFluc mRNA.

| | Particle | | | |
|---|---|---|---|---|
| Lipid | Size | E.E, %, | Zeta | Zeta |
| Composition | (nm) | Stdev | pH 5 | pH 7 |
| KC3-OA | 102.4 | 82.2 ± 2.0 | 22.9 | 1.0 |
| KC2-X1-PH5 | 98.1 | 80.8 ± 3.2 | 22.6 | −3.6 |
| KC3-X1-K | 132.7 | 67.6 ± 2.0 | 24.3 | 2.0 |
| KC3-X1-L | 129.9 | 65.0 ± 2.1 | 23.6 | 1.8 |
| KC3-X1-M | 129.4 | 66.2 ± 1.9 | 26.1 | 2.7 |
| KC3-X1-PH5 | 115.3 | 69.5 ± 1.9 | 22.7 | −0.5 |
| KC3-X1-PH5B | 110.3 | 72.9 ± 3.0 | 25.1 | −1.6 |

The formulations ranged in size from approximately 98 to 133 nm in diameter. The % entrapped mRNA ranged from 65-84% and LNPs exhibited positive surface charge at pH 5 (~+23 to +26 mV) and a negative to close to neutral charge at pH 7 (−4 to +3 mV)

TABLE 7C-3
Luciferase expression (ng/million cells ± stdev) in MutuDC
1940 cells after exposure to LNPs for 24 h at 0.1 μg/mL
total (encapsulated + non-encapsulated) mRNA concentration.

| | Luc activity | | | ICL/KC3- |
|---|---|---|---|---|
| Lipid | (Non-PS | Luc activity | PS/Non-PS | OA |
| Composition | containing) | (PS-containing) | Luc Ratio | activity |
| KC3-OA | 16.8 ± 2.7 | 201.3 ± 34.2 | 12.0 | 1.0 |
| KC2-X1-PH5 | 2.8 ± 0.3 | 226.8 ± 17.6 | 79.9 | 1.1 |
| KC3-X1-K | 1.0 ± 0.2 | 325.2 ± 21.1 | 334.3 | 1.6 |
| KC3-X1-L | 2.6 ± 0.3 | 284.2 ± 13.4 | 110.6 | 1.4 |
| KC3-X1-M | 1.6 ± 0.2 | 291.0 ± 19.0 | 176.9 | 1.4 |
| KC3-X1-PH5 | 4.8 ± 0.2 | 240.7 ± 20.8 | 49.9 | 1.2 |
| KC3-X1-PH5B | 4.4 ± 0.2 | 251.6 ± 28.8 | 57.7 | 1.2 |

TABLE 7C-4
Luciferase expression (ng/million cells ± stdev) in MutuDC
1940 cells after exposure to LNPs for 24 h at 0.3 μg/mL
total (encapsulated + non-encapsulated) mRNA concentration.

| | Luc | | | ICL/KC3- |
|---|---|---|---|---|
| Lipid | activity | Luc activity | PS/Non-PS | OA |
| Composition | (Non-PS) | (PS-containing) | Luc Ratio | activity |
| KC3-OA | 65.1 ± 14.3 | 249.0 ± 15.5 | 3.8 | 1.0 |
| KC2-X1-PH5 | 8.1 ± 0.6 | 294.5 ± 13.3 | 36.2 | 1.5 |
| KC3-X1-K | 2.2 ± 0.1 | 469.4 ± 30.2 | 210.6 | 2.3 |
| KC3-X1-L | 8.3 ± 1.0 | 492.9 ± 19.9 | 59.3 | 2.4 |
| KC3-X1-M | 4.7 ± 0.8 | 388.6 ± 18.2 | 83.0 | 1.9 |
| KC3-X1-PH5 | 18.0 ± 0.9 | 366.0 ± 25.4 | 20.4 | 1.8 |
| KC3-X1-PH5B | 21.5 ± 1.0 | 314.4 ± 5.1 | 14.6 | 1.6 |

### Example 7D Evaluating LNPs Comprising KC3-OA, C16 Phyt-DM3, KC2-X1-H, KC2-X1-PH1, KC2-X1-PH4, KC3-X1-PH2, KC3-X1-S, KC4-X1-H and KC4-X1-PH1 ICLs, with and without PS-Targeting

The ICLs KC3-OA, C16 Phyt-DM3, KC2-X1-H, KC2-X1-PH1, KC2-X1-PH4, KC3-X1-PH2, KC3-X1-S, KC4-X1-H and KC4-X1-PH1 were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc) or these ICLs were separately formulated without DPPS in the composition ICL/DSPC/Chol/PEG-DMG (52/10/36.5/1.5 mol %). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 7D-1
Physicochemical properties and characterization
of DPPS containing LNPs containing FFluc mRNA.

| | Particle | E.E, %, | Zeta | Zeta |
|---|---|---|---|---|
| Lipid Composition | Size (nm) | Stdev | pH 5 | pH 7 |
| KC3-OA | 103.2 | 91.2 ± 3.2 | 24.08 | 1.30 |
| C16-Phyt-DM3 | 107.2 | 97.9 ± 11.4 | 24.37 | 1.63 |
| KC2-X1-H | 105.0 | 78.2 ± 4.0 | 23.96 | −5.77 |
| KC2-X1-PH1 | 92.3 | 89.5 ± 1.3 | 22.53 | −3.66 |
| KC2-X1-PH4 | 105.0 | 97.1 ± 5.6 | 23.79 | −2.10 |
| KC3-X1-PH2 | 127.6 | 95.4 ± 2.6 | 23.61 | −1.04 |
| KC3-X1-S | 109.8 | 87.6 ± 3.2 | 25.08 | −3.82 |
| KC3-X1-H | 129.8 | 95.2 ± 7.3 | 24.68 | −1.28 |
| KC4-X1-PH1 | 123.6 | 97.7 ± 7.1 | 22.03 | −2.04 |

The formulations ranged in size from approximately 92 to 130 nm in diameter. The % entrapped mRNA ranged from 78-98% and LNPs exhibited positive surface charge at pH 5 (~+22 to +25 mV) and a negative to close to neutral charge at pH 7 (−6 to +2 mV).

TABLE 7D-2
Physicochemical properties and characterization of
non-DPPS containing LNPs containing FFluc mRNA.

| | Particle Size | E.E, %, | Zeta | Zeta |
|---|---|---|---|---|
| Lipid Composition | (nm) | Stdev | pH 5 | pH 7 |
| KC3-OA | 105.9 | 94.7 ± 4.5 | 22.82 | 0.98 |
| C16-Phyt-DM3 | 109.6 | 99.0 ± 7.3 | 25.27 | 1.99 |
| KC2-X1-H | 130.9 | 57.3 ± 1.9 | 24.37 | −2.90 |
| KC2-X1-PH1 | 114.6 | 78.2 ± 1.3 | 25.08 | −1.24 |
| KC2-X1-PH4 | 106.6 | 96.9 ± 3.8 | 24.45 | −0.49 |
| KC3-X1-PH2 | 108.8 | 95.2 ± 4.2 | 23.71 | 0.39 |
| KC3-X1-S | 145.9 | 84.6 ± 2.4 | 8.80 | −2.11 |
| KC3-X1-H | 113.9 | 94.8 ± 1.5 | 23.82 | 1.19 |
| KC4-X1-PH1 | 104.4 | 97.0 ± 4.7 | 27.41 | 4.32 |

The formulations ranged in size from approximately 104 to 150 nm in diameter. The 00 entrapped mRNA ranged from 57-9900 and LNPs exhibited positive surface charge at pH 5 (~+9 to +27 mV) and a negative to close to neutral charge at pH 7 (−3 to +4 mV).

TABLE 7D-3
Luciferase expression in MutuDC 1940 cells after exposure to LNPs for 24
h at 0.1 μg/mL total (encapsulated + non-encapsulated) mRNA concentration.

| | Luc activity | Luc activity | | |
|---|---|---|---|---|
| Lipid | (ng/10\hat{ }6 cells) | (ng/10\hat{ }6 cells) | PS/Non-PS | ICL/KC3- |
| Composition | Non-PS | PS-containing | Luc Ratio | OA activity |
| KC3-OA | 11.8 ± 3.2 | 120.3 ± 8.7 | 10.2 | 1.00 |
| C16-Phyt-DM3 | 4.9 ± 1.0 | 58.9 ± 5.2 | 12.1 | 0.49 |
| KC2-X1-H | 7.4 ± 0.6 | 102.7 ± 12.6 | 13.8 | 0.85 |
| KC2-X1-PH1 | 4.5 ± 0.6 | 106.5 ± 19.3 | 23.6 | 0.89 |
| KC2-X1-PH4 | 1.2 ± 0.3 | 140.4 ± 20.2 | 114.2 | 1.17 |
| KC3-X1-PH2 | 4.6 ± 0.5 | 129.2 ± 17.5 | 28.0 | 1.07 |
| KC3-X1-S | 19.9 ± 1.7 | 167.7 ± 10.6 | 8.4 | 1.39 |
| KC3-X1-H | 9.0 ± 0.9 | 184.4 ± 12.8 | 20.6 | 1.53 |
| KC4-X1-PH1 | 2.1 ± 0.4 | 111.9 ± 10.8 | 52.1 | 0.93 |

TABLE 7D-4
Luciferase expression (ng/million cells ± stdev) in MutuDC
1940 cells after exposure to LNPs for 24 h at 0.3 μg/mL
total (encapsulated + non-encapsulated) mRNA concentration.

| | | | | ICL/KC3- |
|---|---|---|---|---|
| Lipid | Luc activity | Luc activity | PS/Non-PS | OA |
| Composition | (Non-PS) | (PS-containing) | Luc Ratio | activity |
| KC3-OA | 57.0 ± 19.2 | 166.4 ± 7.6 | 2.9 | 1.00 |
| C16-Phyt-DM3 | 19.4 ± 4.3 | 145.9 ± 5.0 | 7.5 | 0.88 |
| KC2-X1-H | 27.0 ± 1.1 | 281.8 ± 21.9 | 10.4 | 1.69 |
| KC2-X1-PH1 | 17.9 ± 2.5 | 277.4 ± 22.5 | 15.5 | 1.67 |
| KC2-X1-PH4 | 4.8 ± 0.3 | 316.3 ± 8.1 | 65.9 | 1.90 |
| KC3-X1-PH2 | 19.9 ± 2.5 | 290.4 ± 19.4 | 14.6 | 1.74 |
| KC3-X1-S | 68.7 ± 8.8 | 378.4 ± 26.8 | 5.5 | 2.27 |
| KC3-X1-H | 60.8 ± 5.4 | 333.5 ± 22.3 | 5.5 | 2.00 |
| KC4-X1-PH1 | 9.2 ± 1.5 | 249.2 ± 14.4 | 27.1 | 1.50 |

### Example 7E Evaluating LNPs Comprising KC3-X—K, AKG-C20 (8-9; cis-8-Eicosenoic Acid FA) DM2, AKG-C20 (9-10; gadoleic acid) DM2, AKG-C20 Phyt-DM2, AKG-KC3-X1-N, AKG-KC3-X1-O, AKG-KC3-X1-P, AKG-KC3-X1-Q, AKG-KC3-X1-PH2-B andKC3-X1-K ICLs, with and without PS-Targeting

The ICLs KC3-X1-K, AKG-C20 (8-9; cis-8-Eicosenoic Acid FA) DM2, AKG-C20 (9-10; gadoleic acid) DM2, AKG-C20 Phyt-DM2, AKG-KC3-X1-N, AKG-KC3-X1-0, AKG-KC3-X1-P, AKG-KC3-X1-Q, AKG-KC3-X1-PH2-B and KC3-X1-K were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc) or these ICLs were separately formulated without DPPS in the composition ICL/DSPC/Chol/PEG-DMG (52/10/36.5/1.5 mol %). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 7E-1
Physicochemical properties and characterization
of DPPS containing LNPs containing FFluc mRNA.

| | Particle | E.E, %, | Zeta | Zeta |
|---|---|---|---|---|
| Lipid Composition | Size (nm) | Stdev | pH 5 | pH 7 |
| KC3-X1-K | 122.30 | 93.5 ± 2.8 | 28.33 | 3.60 |
| C20 (8-9; cis-8- | 146.80 | 77.4 ± 1.6 | 25.26 | 2.52 |
| Eicosenoic Acid FA) DM2 | | | | |
| C20 (9-10; gadoleic | 116.80 | 93.2 ± 5.3 | 22.01 | 1.92 |
| acid) DM2 | | | | |
| C20 Phyt-DM2 | 86.80 | 90.5 ± 4.2 | 14.59 | −5.66 |
| KC3-X1-N | 78.53 | 36.6 ± 0.7 | 30.34 | 9.02 |
| KC3-X1-O | 135.70 | 84.2 ± 4.0 | 33.59 | 12.58 |
| KC3-X1-P | 114.20 | 92.2 ± 3.3 | 26.97 | 10.27 |
| KC3-X1-Q | 118.00 | 97.1 ± 6.6 | 26.80 | 12.02 |
| KC3-X1-PH2-B | 137.10 | 91.9 ± 3.1 | 22.10 | −3.91 |

The formulations ranged in size from approximately 78 to 147 nm in diameter. The 00 entrapped mRNA ranged from 37-9700 and LNPs exhibited positive surface charge at pH 5 (~+15 to +34 mV) and a negative to positive charge at pH 7 (−6 to +13 mV).

TABLE 7E-2
Physicochemical properties and characterization of
non-DPPS containing LNPs containing FFluc mRNA.

| | Particle | | | |
|---|---|---|---|---|
| | Size | E.E, %, | Zeta | Zeta |
| Lipid Composition | (nm) | Stdev | pH 5 | pH 7 |
| KC3-X1-K | 123.90 | 92.6 ± 4.7 | 21.52 | 3.02 |
| C20 (8-9; cis-8- | 143.10 | 69.7 ± 1.3 | 16.38 | 2.15 |
| Eicosenoic Acid FA) DM2 | | | | |
| C20 (9-10; gadoleic | 112.70 | 87.3 ± 1.3 | 17.48 | 1.01 |
| acid) DM2 | | | | |
| C20 Phyt-DM2 | 100.30 | 83.8 ± 1.0 | 13.62 | −3.01 |
| KC3-X1-N | 147.40 | 57.3 ± 0.8 | 18.75 | 6.44 |
| KC3-X1-O | 155.30 | 79.4 ± 3.3 | 30.80 | 10.99 |
| KC3-X1-P | 114.10 | 92.4 ± 1.8 | 29.47 | 16.81 |
| KC3-X1-Q | 141.90 | 94.4 ± 2.9 | 25.22 | 13.87 |
| KC3-X1-PH2-B | 117.80 | 92.3 ± 2.7 | 24.41 | 3.16 |

The formulations ranged in size from approximately 100 to 155 nm in diameter. The % entrapped mRNA ranged from 57-94% and LNPs exhibited a positive surface charge at pH 5 ranging from +14 to +31 mV and from −6 to +13 mV at pH 7.

TABLE 7E-3
Luciferase expression (ng/million cells ± stdev) in MutuDC 1940
cells after exposure to LNPs for 24 h at 0.1 μg/mL total (encapsulated
+ non-encapsulated) mRNA concentration. A KC3-OA LNP that contains
52 mol % KC3-OA and 5 mol % DPPS described above was included as
a transfection control at both mRNA concentrations tested.

| | Luc activity | Luc activity | PS/Non-PS | ICL/KC3- |
|---|---|---|---|---|
| Lipid Composition | (Non-PS) | (PS-containing) | Luc Ratio | OA activity |
| KC3-X1-K | 0.9 ± 0.3 | 101.4 ± 6.6 | 115.09 | 0.79 |
| C20 (8-9; cis-8- | 0.6 ± 0.2 | 3.4 ± 0.6 | 5.87 | 0.03 |
| Eicosenoic Acid FA) DM2 | | | | |
| C20 (9-10; gadoleic | 2.2 ± 0.5 | 7.7 ± 1.4 | 3.56 | 0.06 |
| acid) DM2 | | | | |
| C20 Phyt-DM2 | 0.7 ± 0.1 | 23.5 ± 3.1 | 33.73 | 0.18 |
| KC3-X1-N | 5.4 ± 0.9 | 0.0 ± 0.0 | 0.00 | 0.00 |
| KC3-X1-O | 4.8 ± 0.5 | 1.2 ± 0.3 | 0.24 | 0.01 |
| KC3-X1-P | 4.2 ± 1.5 | 3.3 ± 0.9 | 0.77 | 0.03 |
| KC3-X1-Q | 1.6 ± 0.7 | 2.8 ± 0.2 | 1.76 | 0.02 |
| KC3-X1-PH2-B | 1.2 ± 0.4 | 74.6 ± 7.3 | 59.97 | 0.58 |
| KC3-OA control | N/A | 128.5 ± 4.2 | | |

TABLE 7E-4
Luciferase expression (ng/million cells ± stdev) in MutuDC
1940 cells after exposure to LNPs for 24 h at 0.3 μg/mL
total (encapsulated + non-encapsulated) mRNA concentration.

| | Luc activity | Luc activity | PS/Non-PS | ICL/KC3-OA |
|---|---|---|---|---|
| Lipid Composition | (Non-PS) | (PS-containing) | Luc Ratio | activity |
| KC3-X1-K | 3.2 ± 0.5 | 308.4 ± 5.0 | 95.5 | 1.5 |
| C20 (8-9; cis-8- | 1.4 ± 0.5 | 16.5 ± 1.8 | 11.7 | 0.1 |
| Eicosenoic Acid FA) DM2 | | | | |
| C20 (9-10; gadoleic | 4.0 ± 0.6 | 48.3 ± 14.6 | 12.1 | 0.2 |
| acid) DM2 | | | | |
| C20 Phyt-DM2 | 2.8 ± 0.5 | 86.0 ± 10.8 | 30.5 | 0.4 |
| KC3-X1-N | 11.6 ± 1.5 | 0.0 ± 0.0 | 0.0 | 0.0 |
| KC3-X1-O | 9.0 ± 1.5 | 5.2 ± 1.0 | 0.6 | 0.0 |
| KC3-X1-P | 7.9 ± 1.3 | 9.4 ± 0.5 | 1.2 | 0.0 |
| KC3-X1-Q | 2.2 ± 0.2 | 5.3 ± 0.6 | 2.4 | 0.0 |
| KC3-X1-PH2-B | 3.4 ± 0.5 | 190.9 ± 9.3 | 56.5 | 0.9 |
| KC3-OA control | | 206.3 ± 3.7 | | |

### Example 7F. In Vitro Transfection of FEAuc-mRNTA LNPs Composed of the Ionizable Lipids KC3-X1-PH12, KC3-X2-PH12, KC3-X1-AA, KC3-X1-PH5 or KC3-OA that were Formulated in the Presence and Absence of 5 Mol % DPPS-NH4

mRNA encoding Ffluc-mRNA was formulated separately into LNPs comprising an ICL/DSPC/Chol/PEG-DMG (52/10/36.5/1.5 mol %) or ICL/DSPC/DPPS-NH₄/Chol/PEG-DMG (52/5/5/36.5/1.5 mol %) formulated at an N/P ratio of 5.25. The LNPs were prepared and characterized as described in Example 3 and 4 respectively.

Particle size refers to the Z-Average particle size after freezing and thawing. Zeta refers to zeta potential measurements at either pH 5 or pH 7 in units of mV. mRNA encapsulation efficiency after freezing & thawing is referred to as (E.E,%).

TABLE 7F-1
Physicochemical properties and characterization of LNPs containing Ffluc mRNA
comparing different ICLs in a PS-targeted and non-targeted form.

| | | | | | Zeta pH | Zeta pH |
|---|---|---|---|---|---|---|
| ICL | Targeted? | Z-Ave | PDI | % E.E | 5 | 7 |
| KC3-X1-PH12 | No PS | 84.54 | 0.006 | 95.71 ± 0.27 | 17.41 | 3.66 |
| KC3-X2-PH12 | No PS | 88.77 | 0.036 | 95.78 ± 0.19 | 17.94 | 2.73 |
| KC3-X1-AA | No PS | 92.58 | 0.051 | 94.37 ± 0.24 | 19.97 | 4.38 |
| KC3-X1-PH5 | No PS | 85.84 | 0.06 | 96.64 ± 0.28 | 20.69 | 4.61 |
| KC3-OA | No PS | 81.68 | 0.077 | 97.20 ± 0.39 | 16.76 | 5.09 |
| KC3-X1-PH12 | 5 mol % PS | 87.15 | 0.065 | 96.78 ± 0.08 | 19.84 | −1.16 |
| KC3-X2-PH12 | 5 mol % PS | 87.65 | 0.017 | 96.69 ± 0.33 | 18.83 | 0.17 |
| KC3-X1-AA | 5 mol % PS | 86.16 | 0.052 | 96.47 ± 0.20 | 19.62 | −1.23 |
| KC3-X1-PH5 | 5 mol % PS | 83.27 | 0.048 | 98.22 ± 0.13 | 19.24 | −2.66 |
| KC3-OA | 5 mol % PS | 82.93 | 0.028 | 97.90 ± 0.09 | 15.77 | −0.89 |

The formulations ranged in size from approximately 82 to 93 nm in diameter The % entrapped mRNA ranged from 94-98% and LNPs exhibited positive surface charge at pH 5~+16-21 and a negative charge at pH 7 −3 to +5 mV.

The activity of the LNPs was evaluated by transfection MutuDC1940 cells in vitro at 0.3 ug/mL mRNA concentration for 24 h as described in Example 6.

TABLE 7F-2
In vitro activity of LNPs containing Ffluc mRNA comparing
different ICLs in a PS-targeted or non-targeted format.

| ICL | Targeted? | Luc (ng/million cells) | T/NT |
|---|---|---|---|
| KC3-X1-PH12 | No PS | 5.03 ± 0.16 | |
| KC3-X2-PH12 | No PS | 8.12 ± 0.55 | |
| KC3-X1-AA | No PS | 6.31 ± 0.22 | |
| KC3-X1-PH5 | No PS | 12.40 ± 1.15 | |
| KC3-OA | No PS | 26.89 ± 4.67 | |
| KC3-X1-PH12 | 5 mol % PS | 334.27 ± 17.96 | 66.5 |
| KC3-X2-PH12 | 5 mol % PS | 447.74 ± 43.72 | 55.2 |
| KC3-X1-AA | 5 mol % PS | 283.73 ± 24.40 | 45.0 |
| KC3-X1-PH5 | 5 mol % PS | 199.92 ± 22.25 | 16.1 |
| KC3-OA | 5 mol % PS | 76.19 ± 6.52 | 2.8 |

It was found that the rank order of activity and the activity of the PS-targeted to non-targeted ratio (highest to lowest) was KC3-X2-PH12>KC3-X1-PH12>KC3-X1-AA>KC3-X1-PH5>KC3-OA.

### Example 8. MRNA Luciferase Expression of LNPs Containing Various PS-Targeted ICLs

TABLE 8-A
Additional compounds tested in Example 8.

| | |
|---|---|
| KC3-OA | 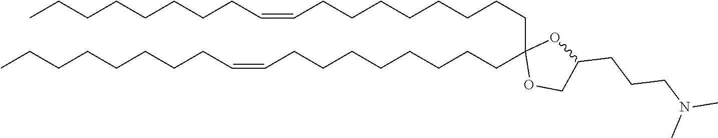 [SMILES: CCCCCCCC/C=C\CCCCCCCCC1(CCCCCCCC/C=C\CCCCCCCC)OCC(CCCN(C)C)O1] |
| C16-Phyt- DM2 | 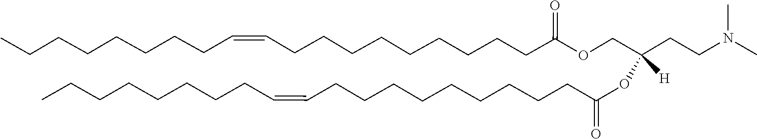 [SMILES: CCCCCCCC/C=C\CCCCCCCCCC(=O)OC[C@H](CCN(C)C)OC(=O)CCCCCCCCC/C=C\CCCCCCCC] |
| C20 (11-12; gondoic acid)-DM2 | 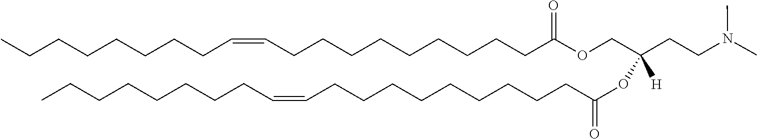 [SMILES: CCCCCCCC/C=C\CCCCCCCCCC(=O)OC[C@H](CCN(C)C)OC(=O)CCCCCCCCC/C=C\CCCCCCCC] |
| C20 (11-12, 14-15; eicosadienoic acid)-DM2 | 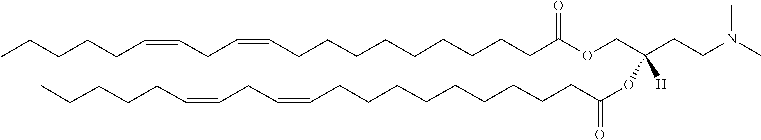 [SMILES: CCCCC/C=C\C/C=C\CCCCCCCCCC(=O)OC[C@H](CCN(C)C)OC(=O)CCCCCCCCC/C=C\C/C=C\CCCCC] |
| C22 (13,14; Erucic acid) DM2 | 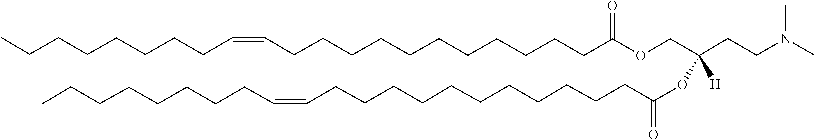 [SMILES: CCCCCCCC/C=C\CCCCCCCCCCCC(=O)OC[C@H](CCN(C)C)OC(=O)CCCCCCCCCCC/C=C\CCCCCCCC] |
| KC3-HA-A | 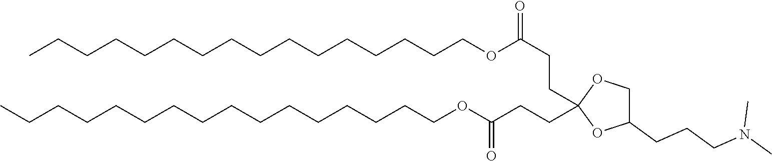 [SMILES: CCCCCCCCCCCCCCCCOC(=O)CCC1(CCC(=O)OCCCCCCCCCCCCCCCC)OCC(CCCN(C)C)O1] |
| KC1-OA | 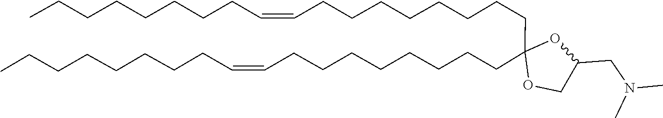 [SMILES: CCCCCCCC/C=C\CCCCCCCCC1(CCCCCCCC/C=C\CCCCCCCC)OCC(CN(C)C)O1] |
| KC2-OA | 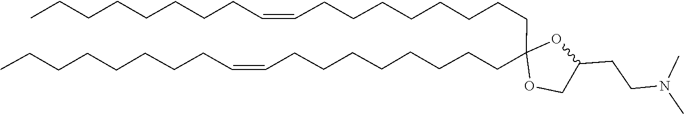 [SMILES: CCCCCCCC/C=C\CCCCCCCCC1(CCCCCCCC/C=C\CCCCCCCC)OCC(CCN(C)C)O1] |
| KC4-OA | 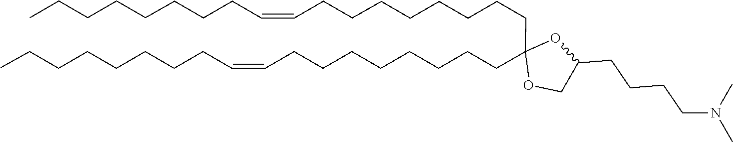 [SMILES: CCCCCCCC/C=C\CCCCCCCCC1(CCCCCCCC/C=C\CCCCCCCC)OCC(CCCCN(C)C)O1] |
| KC3-OA-A | 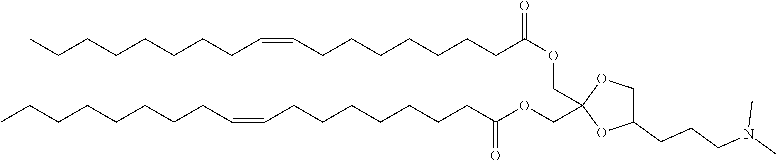 [SMILES: CCCCCCCC/C=C\CCCCCCCC(=O)OCC1(COC(=O)CCCCCCC/C=C\CCCCCCCC)OCC(CCCN(C)C)O1] |
| KC3-OA-B | 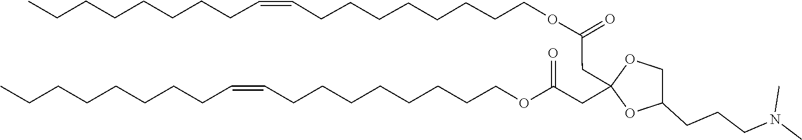 [SMILES: CCCCCCCC/C=C\CCCCCCCCOC(=O)CC1(CC(=O)OCCCCCCCC/C=C\CCCCCCCC)OCC(CCCN(C)C)O1] |
| KC2-OA-C | 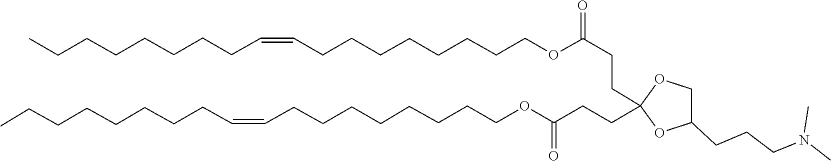 [SMILES: CCCCCCCC/C=C\CCCCCCCCOC(=O)CCC1(CCC(=O)OCCCCCCCC/C=C\CCCCCCCC)OCC(CCCN(C)C)O1] |
| KC3-PA-A | 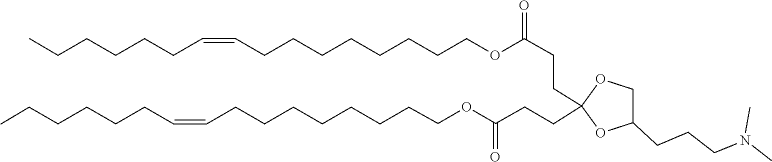 [SMILES: CCCCCC/C=C\CCCCCCCCOC(=O)CCC1(CCC(=O)OCCCCCCCC/C=C\CCCCCC)OCC(CCCN(C)C)O1] |
| KC3-X1-A | 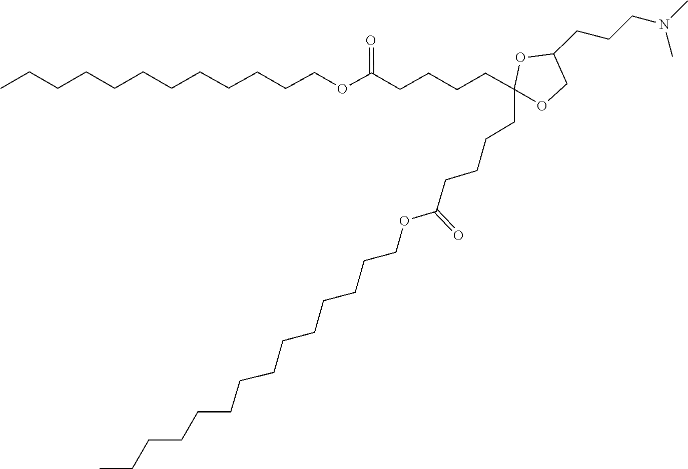 [SMILES: CCCCCCCCCCCCCCOC(=O)CCCCC1(CCCCC(=O)OCCCCCCCCCCCC)OCC(CCCN(C)C)O1] |
| KC2-X2 | 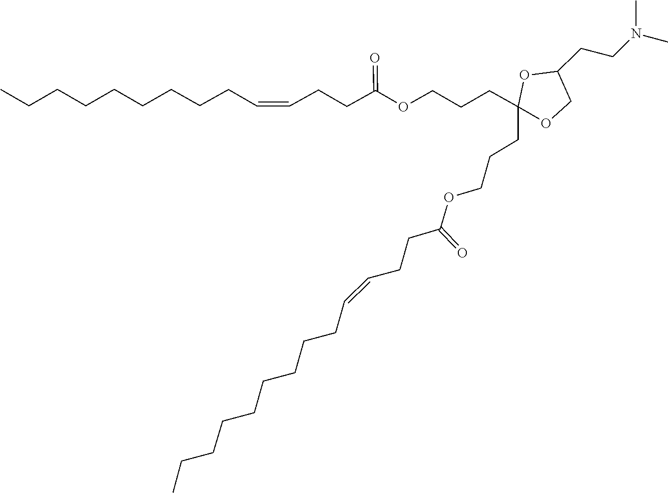 [SMILES: CCCCCCCCC/C=C\CCC(=O)OCCCC1(CCCOC(=O)CC/C=C\CCCCCCCCC)OCC(CCN(C)C)O1] |
| KC3-X2 | 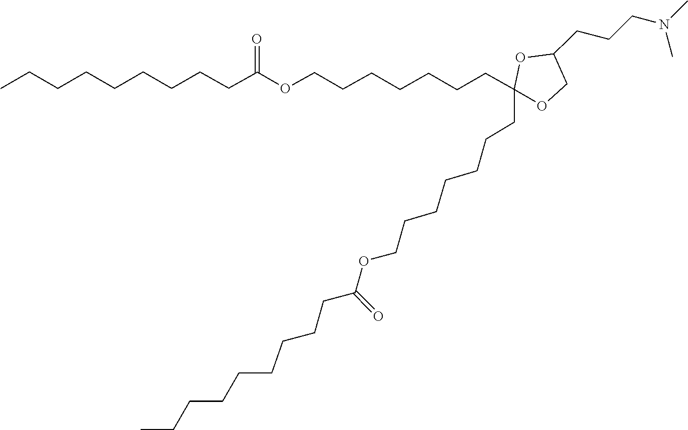 [SMILES: CCCCCCCCCC(=O)OCCCCCCCC1(CCCCCCCOC(=O)CCCCCCCCC)OCC(CCCN(C)C)O1] |
| KC4-X2 | 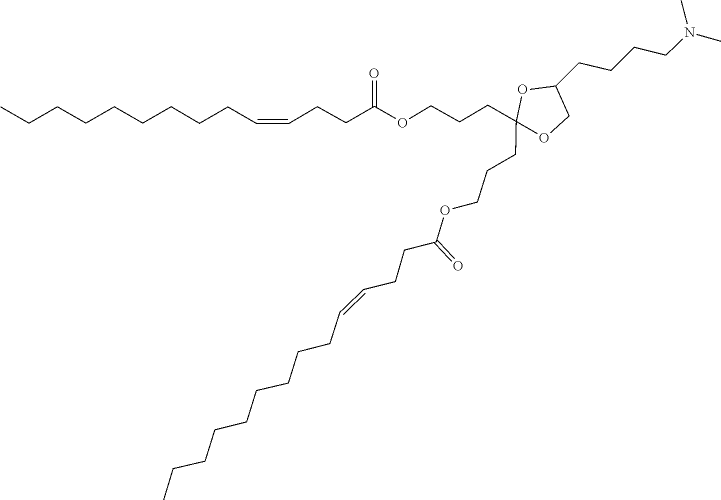 [SMILES: CCCCCCCCC/C=C\CCC(=O)OCCCC1(CCCOC(=O)CC/C=C\CCCCCCCCC)OCC(CCCCN(C)C)O1] |
| KC3-X2-A | 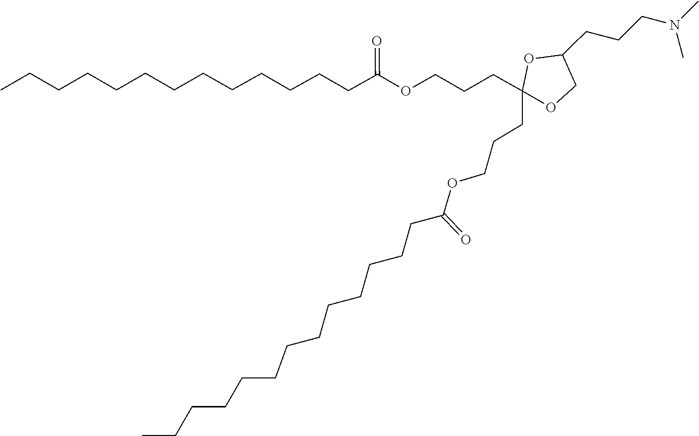 [SMILES: CCCCCCCCCCCCCC(=O)OCCCC1(CCCOC(=O)CCCCCCCCCCCCC)OCC(CCCN(C)C)O1] |
| KC3-X2-B | 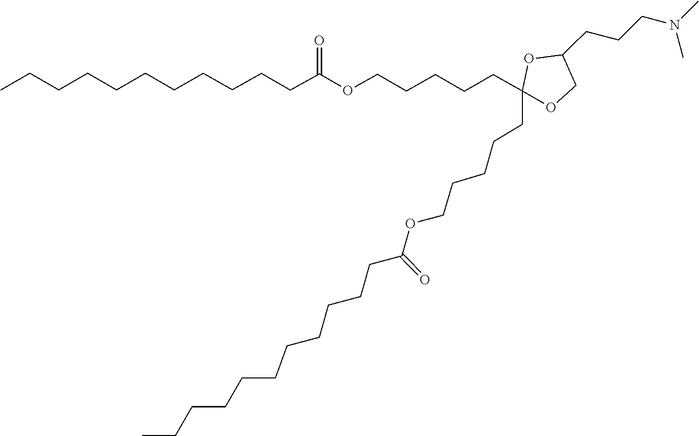 [SMILES: CCCCCCCCCCCC(=O)OCCCCCC1(CCCCCOC(=O)CCCCCCCCCCC)OCC(CCCN(C)C)O1] |
| KC3-X2-C | 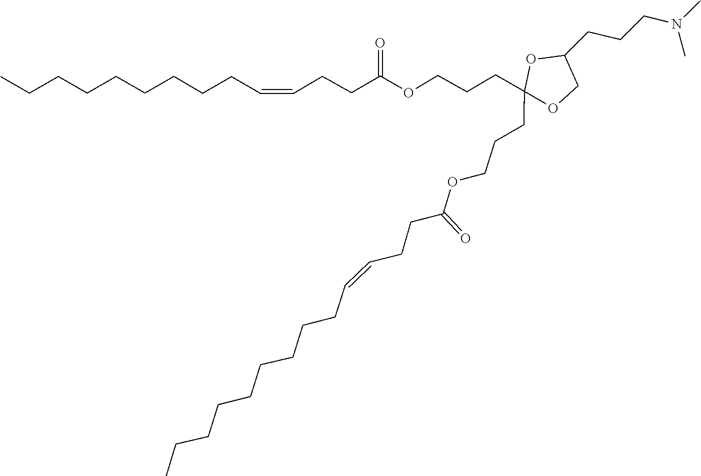 [SMILES: CCCCCCCCC/C=C\CCC(=O)OCCCC1(CCCOC(=O)CC/C=C\CCCCCCCCC)OCC(CCCN(C)C)O1] |
| KC3-X2-C (trans-) | 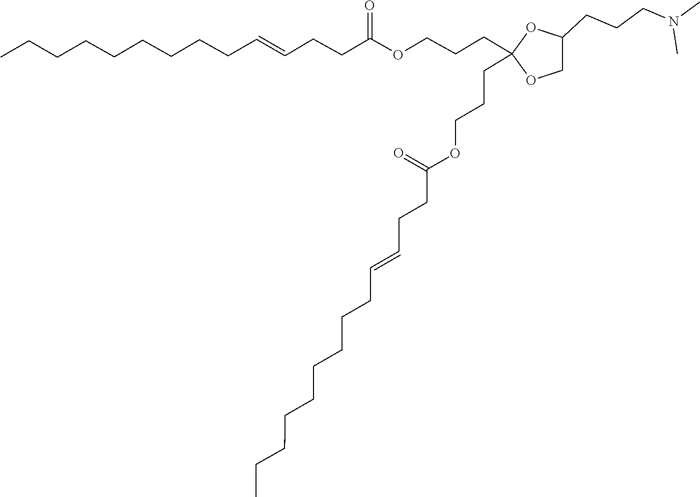 [SMILES: CCCCCCCCC/C=C/CCC(=O)OCCCC1(CCCOC(=O)CC/C=C/CCCCCCCCC)OCC(CCCN(C)C)O1] |
| OA-DM2 | 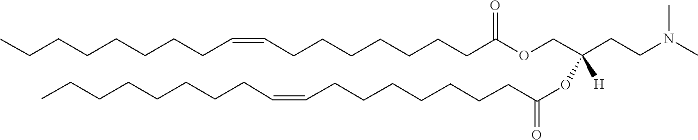 [SMILES: CCCCCCCC/C=C\CCCCCCCC(=O)OC[C@H](CCN(C)C)OC(=O)CCCCCCC/C=C\CCCCCCCC] |
| OA-DM3 | 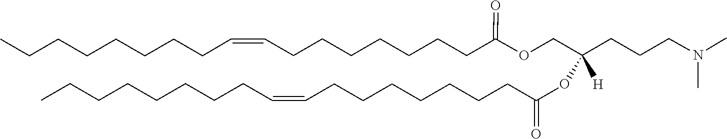 [SMILES: CCCCCCCC/C=C\CCCCCCCC(=O)OC[C@H](CCCN(C)C)OC(=O)CCCCCCC/C=C\CCCCCCCC] |

### Example 8A Evaluating PS-Targeted LNPs Comnprising C16 Phyt-DM2, C22 (13,14, Erucic Acid) DM2, KC3 X1-H, KC3 X1-I, KC3-X1-PH1, KC3-X1-PH3, KC3-X1-PH3B

### ICLs

LNPs were prepared and characterized as described in Examples 3 and 4, except the mRNA formulated was luciferase mRNA (SEQ ID NO: 3 encoding for SEQ ID NO: 2), and included the ICLs: C16 Phyt-DM2, C₂₂ (13,14; Erucic acid) DM2, KC3-X1-H, KC3-X1-I, KC3-X1-PH1, KC3-X1-PH3, KC3-X1-PH3B. Each LNP formulation was prepared with 52 mol % ICL, 5 mol % DSPC, 5 mol % DPPS, 38.5 mol % cholesterol, and 1.5 mol % PEG-DMG with an N/P of 5.25. The KC3-OA formulation was prepared with 48 mol % KC3-OA, 5 mol % DSPC, 5 mol % DPPS, 38.5 mol % cholesterol, and 1.5 mol % PEG-DMG with an N/P of 5.25. The KC3-X1 formulation was prepared with 48 mol % KC3-OA, 5 mol % DSPC, 5 mol % DPPS, 38.5 mol % cholesterol, and 1.5 mol % PEG-DMG with an N/P of 5.25.

Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E,%). The pKa refers to the apparent pKa of the mRNA-LNP particles and the methodology is described in Example 4. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 8A-1
Physicochemical properties and characterization of DPPS-
targeted LNPs made with varying ICLs containing biodegradable
linear and phytyl ICLs.

| | | Particle | | | | |
|---|---|---|---|---|---|---|
| | | Size | | | | |
| | Particle | (nm) | | | | |
| | Size | Post- | E.E, %, | Zeta | Zeta | |
| ICL | (nm) | F/T | stdev | pH 5 | pH 7 | pKa |
| KC3-OA | 117.3 | 117.4 | 84.3 ± 4.8 | 21.4 | −1.1 | 6.99 |
| C16-Phyt- | 90.7 | 92.0 | 87.0 ± 1.9 | 19.0 | −8.1 | 6.48 |
| DM2 | | | | | | |
| C22(13, 14; | 157.6 | 254.5 | 98.4 ± 5.7 | 25.5 | 5.99 | 7.23 |
| Euric acid)- | | | | | | |
| DM2 | | | | | | |
| KC3-X1-H | 136.6 | 135.0 | 84.1 ± 4.0 | 23.0 | −2.7 | 7.34 |
| KC3-X1-I | 121.3 | 122.8 | 87.0 ± 3.8 | 25.8 | −3.7 | 7.35 |
| KC3-X1-PH1 | 117.3 | 119.8 | 96.2 ± 5.7 | 20.8 | −2.3 | 7.27 |
| KC3-X1-PH3 | 150.6 | 150.3 | 97.4 ± 0.7 | 20.4 | 0.64 | 7.69 |
| KC3-X1 | 117.7 | 126.0 | 92.8 ± 6.8 | 19.86 | 1.78 | N.D |

N.D refers to data that was not determined.

TABLE 8A-2
Luciferase mRNA transfection activity of DPPS-
targeted LNPs made with varying of ICLs.

| ICL | Luc (ng/million cells) | Rel. Luc. |
|---|---|---|
| KC3-OA | 243.2 ± 5.9 | 1.00 |
| C16-Phyt-DM2 | 161.9 ± 4.4 | 0.67 |
| C22(13, 14; Euric | 23.8 ± 2.6 | 0.10 |
| acid)-DM2 | | |
| KC3-X1-H | 224.7 ± 16.2 | 0.92 |
| KC3-X1-I | 155.9 ± 10.4 | 0.64 |
| KC3-X1-PH1 | 308.0 ± 23.4 | 1.27 |
| KC3-X1-PH3 | 49.8 ± 6.5 | 0.20 |
| KC3-X1 | 66.7 ± 6.7 | 0.27 |

The ICLs KC3-X1-PH1, KC3-X1-H, C₁₆-Phyt-DM2, KC3-X1-I, KC3-X1-PH1 and KC3-X1-PH3 produced 126.7%, 92.4%, 66.7%, 64.1% relative to the activity of KC3-OA while KC3-X1, KC3-X1-PH3 and C22-DM2 produced lower amounts of luciferase at 27.4%, 20.5% and 9.8% respectively.

### Example 8B Evaluating PS-Targeted LNPs Comprising KC3-OA, OA-DM2E, C20 (11-12, 14-15; Eicosadienoic Acid)-DM2, KC2-X1-G, KC3-X1-J, KC3-X1-PH4, KC3-X1-PH4-B

### ICLs

LNPs were prepared and characterized as described in Examples 3 and 4, except the mRNA formulated was luciferase mRNA (SEQ ID NO: 3 encoding for the polypeptide in SEQ ID NO: 2), and included the ICLs: KC3-OA, OA-DM2E, C20 (11-12, 14-15; eicosadienoic acid)-DM2, KC2-X1-G, KC3-X1-J, KC3-X1-PH4, KC3-X1-PH4-B. Each LNP formulation was prepared with 52 mol % ICL, 5 mol % DSPC, 5 mol % DPPS, 38.5 mol % cholesterol, and 1.5 mol % PEG-DMG with an N/P of 5.25.

Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E,%). The pKa refers to the apparent pKa of the mRNA-LNP particles. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 8B-1
Physicochemical properties and characterization of DPPS-targeted
LNPs made with varying ICLs.

| | | Particle | | | | |
|---|---|---|---|---|---|---|
| | Particle | Size | | | Zeta | |
| | Size | (nm) | E.E, %, ± | Zeta | pH | |
| ICL | (nm) | Post-F/T | stdev | pH 5 | 7 | pKa |
| KC3-OA | 114.0 | 111.80 | 88.0 ± 2.2 | 20.27 | −1.66 | 7.018 |
| OA-DM2E | 118.4 | 121.20 | 50.9 ± 1.9 | 20.54 | −6.94 | 6.975 |
| C20 (11-12; | 132.3 | 307.70 | 98.9 ± 5.6 | 24.09 | 1.25 | 7.49 |
| gondoic acid)- | | | | | | |
| DM2 | | | | | | |
| C20 (11-12, 14- | 120.5 | 194.70 | 97.0 ± 4.6 | 25.95 | −0.25 | 7.271 |
| 15; eicosadienoic | | | | | | |
| acid)-DM2 | | | | | | |
| KC2-X1-G | 114.6 | 117.30 | 64.7 ± 1.7 | 19.60 | −3.80 | 7.007 |
| KC3-X1-J | 102.5 | 104.20 | 93.7 ± 4.2 | 21.77 | −0.65 | N.D |
| KC3-X1-PH4 | 133.3 | 133.50 | 97.8 ± 2.1 | 22.56 | −1.23 | 7.39 |
| KC3-X1-PH4-B | 149.1 | 149.30 | 95.6 ± 5.7 | 27.14 | −5.23 | 7.489 |

TABLE 8B-2
Luciferase mRNA transfection activity of DPPS-
targeted LNPs made with varying of ICLs.

| | Luc | |
|---|---|---|
| | (ng/million | |
| ICL | cells) | Stdev |
| KC3-OA | 312.0 | 33.28 |
| OA-DM2E | 6.15 | 0.23 |
| C20 (11-12; gondoic | 12.9 | 1.64 |
| acid)-DM2 | | |
| C20 (11-12, 14-15; | 14.89 | 1.52 |
| eicosadienoic acid)-DM2 | | |
| KC2-X1-G | 298.98 | 11.28 |
| KC3-X1-J | 268.29 | 12.96 |
| KC3-X1-PH4 | 195.92 | 14.37 |
| KC3-X1-PH4-B | 201.52 | 15.99 |

The LNPs made from the ICLs studied in this example produced LNPs that ranged in size from 104-307 nm. Most had mRNA entrapment efficiencies close to 90% or above, but 2 lipids gave rise to poor entrapment at OA-DM2E at 51% and KC2-X1-G at 65%. All LNPs gave positive zeta potentials ranging from +19.6-27.1 mV and values that were slightly negative or close to zero at pH 7. The ICLs OA-DM2E, C20 (11-12; gondoic acid) DM2, C₂₀ (11-12, 14-15; eicosadienoic acid) DM2, AKG-KC2-X1-G, AKG-KC3-X1-J, AKG-KC3-X1-PH4, AKG-KC3-X1-PH4-B produced 2.0, 4.1, 4.8, 95.8, 86.0, 62.8 and 64.6% respectively relative to the activity of KC3-OA.

### Example 8C Evaluating PS-Targeted LNPs Comprising KC3-OA, KC3-X1-E, KC3-X1-F, KC3-X1-G, KC3-X1-PH1-B, and KC3-X2-B ICLs

LNPs were prepared and characterized as described in Examples 3 and 4, except the mRNA formulated was luciferase mRNA (SEQ ID NO: 3 encoding for SEQ ID NO: 2), and included the ICLs: KC3-OA, KC3-X1-E, KC3-X1-F, KC3-X1-G, KC3-X1-PH1-B, KC3-X2-B.

Each LNP formulation was prepared with 52 mol % ICL, 5 mol % DSPC, 5 mol % DPPS, 38.5 mol % cholesterol, and 1.5 mol % PEG-DMG with an N/P of 5.25.

Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E,%). The pKa refers to the apparent pKa of the mRNA-LNP particles. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 8C-1
Physicochemical properties and characterization of DPPS-targeted
LNPs made with varying ICLs.

| | Particle | Particle | | | Zeta | |
|---|---|---|---|---|---|---|
| | Size | Size (nm) | E.E, %, ± | Zeta | pH | |
| ICL | (nm) | Post-F/T | stdev | pH 5 | 7 | pKa |
| KC3-OA | 120.2 | 119.9 | 79.06 ± 6.6 | 16.53 | −4.801 | 6.985 |
| KC3-X1-E | 134.7 | 136.5 | 94.78 ± 6.9 | 15.71 | −1.9 | 7.496 |
| KC3-X1-F | 128.2 | 127.7 | 91.98 ± 5.2 | 23.23 | −2.149 | 7.413 |
| KC3-X1-G | 115.9 | 118.1 | 96.40 ± 7.2 | 20.18 | −0.2 | 7.411 |
| KC3-X1-PH1-B | 129.6 | 129.2 | 82.41 ± 1.8 | 21.73 | −6.605 | 7.285 |
| KC3-X2-B | 149.1 | 170.5 | 84.93 ± 2.23 | 17.38 | 4.2 | 7.796 |

TABLE 8C-2
Luciferase mRNA transfection activity of DPPS-
targeted LNPs made with varying of ICLs.

| ICL | Luc (ng/million cells) |
|---|---|
| KC3-OA | 190.37 ± 14.2 |
| KC3-X1-E | 69.01 ± 5.88 |
| KC3-X1-F | 88.72 ± 11.24 |
| KC3-X1-G | 131.59 ± 16.62 |
| KC3-X1-PH1-B | 164.78 ± 14.93 |
| KC3-X2-B | 0.92 ± 0.14 |

The LNPs that were made from the ICLs used in this study, produced LNPs with sizes ranging from 116-149 nm and mRNA entrapment ranging from 79-96%. The zeta potential at pH 5 were highly cationic and closer to zero at pH 7. Interestingly KC3-X2-B was the only formulation with a positive zeta at pH 7 (+4.2), and it correlated with the lowest Luciferase mRNA transfection activity at <1 ng/million cells, 207-times lower than KC3-OA which had a zeta potential at pH 7 of −4.8.

The ICLs KC3-X1-E, KC3-X1-F, KC3-X1-G, KC3-X1-PH1-B and KC3-X2-B produced 36.3%, 46.6%, 69.1%, 86.6%, and 0.9% respectively relative to the activity of KC3-OA.

### Example 8D Evaluating PS-Targeted LNPs Comprising KC3-OA, KC3-X2, OA-DM2, OA-DM3, KC3-HA-A, KC3-X1-A, KC2-OA-C, KC2-X2, KC3-PA-A, KC3-X2-A, KC3-X2-C, KC2-OA, KC4-OA, KC3-X2-B and KC1-OA ICLs

LNPs were prepared and characterized as described in Examples 3 and 4, except the mRNA formulated was luciferase mRNA (SEQ ID NO: 3 encoding for SEQ ID NO: 2), and included the ICLs: KC3-OA, KC3-X2, OA-DM2, OA-DM3, KC3-HA-A, KC3-X1-A, KC2-OA-C, KC2-X2, KC3-PA-A, KC3-X2-A, KC3-X2-C, KC2-OA, KC4-OA, KC3-X2-B and KC1-OA.

Each LNP formulation was prepared with 52 mol % ICL, 5 mol % DSPC, 5 mol % DPPS, 38.5 mol % cholesterol, and 1.5 mol % PEG-DMG with an N/P of 5.25.

Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E,%). The pKa refers to the apparent pKa of the mRNA-LNP particles as described in Example 7. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 8D-1
Physicochemical properties and characterization of DPPS-
targeted LNPs made with varying ICLs.

| | | Particle | | | | |
|---|---|---|---|---|---|---|
| | | Size | E.E, %, ± | | | |
| | Particle | (nm) | stdev | | | |
| | Size | Post- | (post | Zeta | Zeta | |
| ICL | (nm) | F/T | F/T) | pH 5 | pH 7 | pKa |
| KC3-OA | 123.2 | 124 | 97.6 ± 9.2 | 22.9 | 0.4703 | 7.0 |
| KC3-X2 | 102.4 | 176.5 | 84.0 ± 3.9 | 24.9 | 5.2 | 6.3 |
| OA-DM2 | 112.4 | 114.4 | 94.2 ± 1.9 | 22.5 | 1.1 | 7.3 |
| OA-DM3 | 106.5 | 211.9 | 94.9 ± 7.3 | 17.1 | 4.1 | 7.83 |
| KC3-HA-A | 144.6 | 144.2 | 93.5 ± 6.2 | 17.6 | 11.9 | 7.0 |
| KC3-X1-A | 122.2 | 138.3 | 95.1 ± 8.9 | 17.6 | 8.2 | 7.8 |
| KC2-OA-C | 115.4 | 116.8 | 95.1 ± 5.9 | 25.2 | 3.0 | 7.3 |
| KC2-X2 | 94.41 | 232.4 | 90.1 ± 6.4 | 20.6 | 3.0 | 7.6 |
| KC3-PA-A | 105.5 | 200.4 | 94.8 ± 4.3 | 22.0 | 6.0 | 7.9 |
| KC3-X2-A | 119.8 | 121.8 | 95.7 ± 6.1 | 19.6 | 13.1 | 8.04 |
| KC3-X2-C | 98.79 | 125.5 | 95.6 ± 4.8 | 18.6 | 4.79 | 8.12 |
| KC2-OA | 92.36 | 90.6 | 85.1 ± 6.3 | 19.2 | −6.34 | 6.58 |
| KC4-OA | 126.9 | 127.0 | 97.7 ± 3.4 | 29.1 | −0.50 | 7.22 |
| KC3-X2-B | 103.8 | 126.5 | 94.6 ± 2.7 | 23.57 | 5.009 | 7.83 |
| KC1-OA | 86.66 | 88.05 | 76.2 ± 2.8 | 10.04 | −15.33 | 5.82 |

The size range of the LNPs described in Table 81D-1 ranged from 92.4-144.6 nm before freezing and thawing. After a F/T event the samples increased in size between 0.08-59.4%. The mRNA encapsulation efficiency was between 76.2-97.7% for all samples. The range of zeta potential values at pH 5 was 10.0-29.13 mV while at pH 7 the range was −15.3 to +13.1.

TABLE 8D-2
Luciferase mRNA transfection activity of DPPS-
targeted LNPs made with varying of ICLs.

| ICL | Luc (ng/million cells) | Luc Rel. to KC3-OA |
|---|---|---|
| KC3-OA | 112.58 ± 13.69 | 1.00 |
| KC3-X2 | 3.24 ± 1.59 | 0.03 |
| OA-DM2 | 18.10 ± 9.73 | 0.16 |
| OA-DM3 | 5.28 ± 11.38 | 0.05 |
| KC3-HA-A | 0.09 ± 0.06 | 0.00 |
| KC3-X1-A | 1.58 ± 0.19 | 0.01 |
| KC2-OA-C | 21.04 ± 5.25 | 0.19 |
| KC2-X2 | 0.70 ± 0.14 | 0.01 |
| KC3-PA-A | 3.74 ± 1.02 | 0.03 |
| KC3-X2-A | BLLOQ | — |
| KC3-X2-C | 0.16 ± 0.10 | <0.01 |
| KC2-OA | 79.21 ± 7.54 | 0.70 |
| KC4-OA | 84.09 ± 10.62 | 0.75 |
| KC3-X2-B | 0.62 ± 0.21 | 0.01 |
| KC1-OA | 5.08 ± 2.42 | 0.05 |

The most active ICLs in this experiment were in order KC3-OA, KC4-OA, KC2-OA, KC2-OA-C, OA-DM2, OA-DM3, KC1-OA, KC3-PA-A, KC3-X2, KC3-X1-A, KC2-X2, KC3-X2-B, KC3-X2-C, KC3-HA-A, KC3-X2-A.

### Example 8E Evaluating PS-Targeted LNPs Comprising KC3-OA, KC3-X2, OA-DM2, OA-DM3, KC3-HA-A. KC3-X1-A. KC2-OA-C. KC2-X2. KC3-PA-A. KC3-X2-A. KC3-X2-C. KC2-OA. KC4-OA, KC3-X2-B and KC1-OA ICLs

LNPs were prepared and characterized as described in Examples 3 and 4, except the mRNA formulated was luciferase mRNA (SEQ ID NO: 3 encoding for SEQ ID NO: 2), and included the ICLs: AKG-KC3-OA-A, AKG-KC3-OA-B, AKG-KC3-X2-C, AKG-KC3-X2-C(trans-), AKG-KC4-X2 and KC3-OA. Each LNP formulation was prepared with 52 mol % ICL, 5 mol % DSPC, 5 mol % DPPS, 38.5 mol % cholesterol, and 1.5 mol % PEG-DMG with an N/P of 5.25. Particle size refers to the Z-Average particle size. Post-F/T refers to samples that were measured after a freeze/thaw event, and Zeta refers to zeta potential measurements at either pH 5 or pH 7 in units of mV. mRNA encapsulation efficiency is referred to as (E.E,%). The pKa refers to the apparent pKa of the mRNA-LNP particles as described in Example 7. Post-F/T refers to samples that underwent a freeze-thaw cycle to evaluate their stability (20 min at −80° C., thawing at 37° C.). BLOQ refers to below limit of quantitation. Transfection efficiency was determined in murine dendritic cells of LNPs using a firefly luciferase assay as described in Example 6A.

TABLE 8E-1
Physicochemical properties and characterization of DPPS-
targeted LNPs made with varying ICLs.

| | | Particle | | | | |
|---|---|---|---|---|---|---|
| | | Size | | | | |
| | Particle | (nm) | E.E, %, ± | | | |
| | Size | Post- | H | Zeta | Zeta | |
| ICL | (nm) | F/T | stdev | pH 5 | pH 7 | pKa |
| KC3-OA | 123.2 | 124 | 97.6 ± 9.2 | 22.89 | 0.5 | 7.0 |
| (control) | | | | | | |
| KC3-OA-A | 135.9 | 155 | 97.6 ± 14.4 | 13.58 | 6.7 | 7.8 |
| KC3-OA-B | 132.8 | 144.8 | 97.0 ± 17.0 | 18.4 | 10.0 | 8.1 |
| KC3-X2-C | 135.5 | 207.8 | 96.3 ± 7.5 | 9.4 | 5.6 | 8.1 |
| KC3-X2-C | 162.9 | 165.3 | 96.9 ± 3.7 | 15.6 | 11.8 | 8.1 |
| (trans-) | | | | | | |
| KC4-X2 | 124.2 | 160.1 | 96.7 ± 6.2 | 11.05 | 5.806 | 8.1 |

The size range of the LNPs described in Table 7A-1 ranged from 124.2-162.9 nm. Upon freezing and thawing, the samples changed in size between 1.4-34.8%. The mRNA encapsulation efficiency was between 96.3-97.6% for all samples. Upon freezing and thawing, the samples changed less than 1.5% in % E.E. The range of zeta potential values at pH 5 was 9.4-22.9 mV while at pH 7 the range was 0.47-11.8. The pKa of KC3-OA was determined to be 7.0 while the other ICLs had a higher pKa of between 7.8-8.1. KC3-OA was highly cationic at low pH with a value of 22.9 while having a close to neutral value of 0.47. The other ICLs tested produced LNPs with moderate positive zeta potential values at pH 5 and values greater than +5.5 mV at pH 7. The difference between the pH 5 and 7 values was +22.4 mV for KC3-OA and between 3.2 and 5.8-times lower for the other ICLs tested indicating that they did not produce as much pH-dependent charge switching compared to KC3-OA in this study.

TABLE 8E-2
Luciferase mRNA transfection activity of DPPS-
targeted LNPs made with varying of ICLs.

| ICL | Luc (ng/million cells) | Luc relative to KC3-OA |
|---|---|---|
| KC3-OA | 155.7 ± 0.13 | 1.00 |
| KC3-OA-A | 0.77 ± 0.19 | <0.01 |
| KC3-OA-B | 0.63 ± 0.23 | <0.01 |
| KC3-X2-C | BLOQ | <0.01 |
| KC3-X2-C (trans-) | 0.27 ± 0.12 | <0.01 |
| KC4-X2 | 0.21 ± 0.11 | <0.01 |

The Luciferase mRNA transfection activity was negligible of all samples tested in this study compared to KC3-OA.

### Example 8F Evaluating PS-Targeted LNPs Comprising DM2, KC2-X1-PH5, KC3-X1-K, KC3-X1-L, KC3-X1-M, KC3-X1-PH5 and KC3-X1-PH5B ICLs

LNPs were prepared and characterized as described in Examples 3 and 4, except the mRNA formulated was luciferase mRNA (SEQ ID NO: 3 encoding for SEQ ID NO: 2), and included the ICLs: DM2, KC2-X1-PH5, KC3-X1-K, KC3-X1-L, KC3-X1-M, KC3-X1-PH5 and KC3-X1-PH5B.

PS-Targeted LNPs were prepared with 5 mol % DSPC, 5 mol % DPPS, 1.5 mol % PEG-DMG, and 52 mol % ICL with 36.5 mol % of cholesterol and with an N/P of 5.25. Post-F/T refers to samples that underwent a freeze-thaw cycle to evaluate their stability (20 min at −80° C., thawing at 37° C.). Transfection efficiency was determined in murine dendritic cells of LNPs using a firefly luciferase assay as described in Example 6A.

TABLE 8F-1
Particle size refers to the Z-Average particle size.

| | Post Filter | | Post F/T | |
|---|---|---|---|---|
| | Z-Average | | Z-Average | |
| ICL | (nm) | PDI | (nm) | PDI |
| KC3-OA | 112.1 | 0.131 | 112.3 | 0.1025 |
| KC2-X1-PH5 | 103.5 | 0.096 | 102 | 0.1287 |
| KC3-X1-K | 115.2 | 0.1086 | 117.8 | 0.1102 |
| KC3-X1-L | 111 | 0.092 | 112 | 0.1069 |
| KC3-X1-M | 105.9 | 0.1167 | 109.2 | 0.09507 |
| KC3-X1-PH5 | 110.1 | 0.1329 | 109 | 0.1298 |
| KC3-X1-PH5B | 120.8 | 0.144 | 120.5 | 0.1804 |

TABLE 8F-2
Zeta refers to zeta potential measurements at
either pH 5 or pH 7 in units of mV. mRNA encapsulation
efficiency is referred to as (E.E, %).

| | % E.E | % E.E | zeta | zeta |
|---|---|---|---|---|
| ICL | filtered | F/T | pH 5 | pH 7 |
| KC3-OA | 85.38 ± 0.92 | 84.65± | 21.24 | 2.053 |
| KC2-X1-PH5 | 81.77 ± 2.87 | 81.25 ± 2.89 | 23.35 | −6.245 |
| KC3-X1-K | 89.50 ± 3.87 | 83.16 ± 4.18 | 29.17 | −0.1245 |
| KC3-X1-L | 89.64 ± 2.15 | 82.44 ± 3.42 | 23.68 | −0.01582 |
| KC3-X1-M | 90.22 ± 1.67 | 88.00 ± 4.13 | 30.2 | 1.071 |
| KC3-X1-PH5 | 94.77 ± 3.05 | 94.48 ± 4.61 | 28.05 | −2.584 |
| KC3-X1-PH5B | 85.05 ± 3.3 | 84.13 ± 3.17 | 26.28 | −7.084 |

The size range of the LNPs described in Table 8F-1 ranged from 103.5-120.8 nm. Upon freezing and thawing, the samples changed in size between 0-3.3%. The mRNA encapsulation efficiency shown in Table 8F-2 was between 81.8 and 94.8% for all samples. Upon freezing and thawing, the samples changed in % E.E between 0 and 7.2%. The zeta potential range at pH 5 was strongly positive at 21.2-30.2 mV and at pH 7 was −7.1 to 2.1 mV indicating that the LNPs were positively charged at pH 5 and either slightly negative or slightly positively charged at pH 7.

TABLE 8F-3
Luciferase mRNA transfection activity of PS-targeted LNPs

| | Luc | Luc Relative to |
|---|---|---|
| ICL | ng/10\hat{ }6 cells | KC3-OA/PS |
| Control KC3-OA/PS | 147.26 ± 4.74 | 1.00 |
| KC3-OA | 126.9 ± 5.0 | 0.86 |
| KC2-X1-PH5 | 185.9 ± 6.0 | 1.26 |
| KC3-X1-K | 271.9 ± 27.1 | 1.85 |
| KC3-X1-L | 211.2 ± 24.6 | 1.43 |
| KC3-X1-M | 223.6 ± 36.5 | 1.52 |
| KC3-X1-PH5 | 254.6 ± 34.1 | 1.73 |
| KC3-X1-PH5B | 197.9 ± 19.9 | 1.34 |

The luc expression range was 126.9-271.9 ng/million cells for the LNP samples shown in Example 23. The rank order of the ICLs in this study from most active to least active was KC3-X1-K, KC3-X1-PH5, KC3-X1-M, KC3-X1-L, KC3-X1-PH5B, KC2-X1-PH5, KC3-OA. The control sample was KC3-OA/PS study with 48 mol % ICL from Table 7A-1 that was frozen down into multiple aliquots and freshly thawed for this study.

### Example 8G Evaluating PS-Targeted LNPs Comprising KC2-X—K, KC3-X1-PH6, KC3-X1-PH8, KC3-X1-PH9, KC3-X1-R, KC3-X1-T, KC3-X1-U, KC4-X1-PH4, KC3-X1-PH7, KC3-X1-V, KC3-OA (Control) ICLs

The ICLs KC2-X1-K, KC3-X1-PH6, KC3-X1-PH8, KC3-X1-PH9, KC3-X1-R, KC3-X1-T, KC3-X1-U, KC4-X1-PH4, KC3-X1-PH7, KC3-X1-V, KC3-OA (Control) were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8G-1
Physicochemical properties and characterization
of DPPS containing LNPs containing FFluc mRNA.

| | Particle Size | E.E, %, | | |
|---|---|---|---|---|
| Lipid Composition | (nm) | Stdev | Zeta pH 5 | Zeta pH 7 |
| KC2-X1-K | 140.60 | 44.1 ± 2.2 | 19.00 | −1.68 |
| KC3-X1-PH6 | 105.70 | 83.0 ± 4.3 | 21.16 | −1.06 |
| KC3-X1-PH8 | 103.10 | 86.8 ± 2.7 | 18.16 | −2.66 |
| KC3-X1-PH9 | 107.70 | 82.3 ± 2.0 | 19.31 | −2.14 |
| KC3-X1-R | 143.90 | 93.6 ± 4.1 | 20.16 | −2.37 |
| KC3-X1-T | 100.80 | 44.8 ± 3.5 | 0.45 | −5.87 |
| KC3-X1-U | 98.86 | 91.0 ± 3.8 | 19.02 | −2.28 |
| KC4-X1-PH4 | 100.20 | 97.8 ± 3.5 | 21.80 | −1.07 |
| KC3-X1-PH7 | 98.14 | 86.0 ± 3.6 | 16.36 | −2.46 |
| KC3-X1-V | 129.90 | 92.2 ± 5.2 | 12.82 | −3.80 |
| KC3-OA | 107.70 | 84.4 ± 2.3 | 15.25 | −2.15 |

The formulations ranged in size from approximately 98 to 144 nm in diameter. The % entrapped mRNA ranged from 44-98% and LNPs exhibited positive surface charge at pH 5 (~+13 to +22 mV) and a negative charge at pH 7 (−1.1 to −2.5 mV).

TABLE 8G-2
Luciferase expression in MutuDC 1940 cells after
exposure to LNPs for 24 h at 0.1 μg/mL total
(encapsulated + non-encapsulated) mRNA concentration.

| Lipid | Luc activity (ng/10\hat{ }6 | ICL/KC3-OA |
|---|---|---|
| Composition | cells) | activity |
| KC2-X1-K | 88.2 ± 14.5 | 0.92 |
| KC3-X1-PH6 | 110.9 ± 9.9 | 1.16 |
| KC3-X1-PH8 | 110.6 ± 8.9 | 1.16 |
| KC3-X1-PH9 | 60.2 ± 3.1 | 0.63 |
| KC3-X1-R | 19.5 ± 1.1 | 0.20 |
| KC3-X1-U | 63.8 ± 4.8 | 0.67 |
| KC4-X1-PH4 | 18.9 ± 1.1 | 0.20 |
| KC3-X1-PH7 | 37.1 ± 0.8 | 0.39 |
| KC3-X1-V | 43.4 ± 5.4 | 0.46 |
| KC3-OA | 95.4 ± 6.0 | 1.00 |

TABLE 8G-3
Luciferase expression in MutuDC 1940 cells after
exposure to LNPs for 24 h at 0.3 μg/mL total
(encapsulated + non-encapsulated) mRNA concentration.

| | Luc activity (ng/10\hat{ }6 | ICL/KC3-OA |
|---|---|---|
| Lipid Composition | cells) ± stdev | activity |
| KC2-X1-K | 262.7 ± 15.3 | 1.24 |
| KC3-X1-PH6 | 285.5 ± 21.4 | 1.35 |
| KC3-X1-PH8 | 279.2 ± 7.7 | 1.32 |
| KC3-X1-PH9 | 178.1 ± 20.6 | 0.84 |
| KC3-X1-R | 80.3 ± 4.7 | 0.38 |
| KC3-X1-U | 212.5 ± 19.0 | 1.01 |
| KC4-X1-PH4 | 102.1 ± 4.7 | 0.48 |
| KC3-X1-PH7 | 144.0 ± 8.6 | 0.68 |
| KC3-X1-V | 168.6 ± 10.4 | 0.80 |
| KC3-OA | 211.2 ± 13.2 | 1.00 |

### Example 8H Evaluating PS-Targeted LNPs Comprising ICLs KC3-X2-R, KC3-X2-K9, KC3-X1-PH10, KC3-X1-PH11, KC3-X1-PH12, KC3-X1-W, KC3-X2-K and KC3-OA (Control) ICLs

The ICLs KC3-X2-R, KC3-X2-K9, KC3-X1-PH10, KC3-X1-PH11, KC3-X1-PH12, KC3-X1-W, KC3-X2-K and KC3-OA (Control) were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8H-1
Physicochemical properties and characterization
of DPPS containing LNPs containing FFluc mRNA

| Lipid | Particle | E.E, %, | | |
|---|---|---|---|---|
| Composition | Size (nm) | Stdev | Zeta pH 5 | Zeta pH 7 |
| KC3-X2-R | 154.60 | 95.10 ± 6.92 | 21.75 | 1.02 |
| KC3-X2-K9 | 107.70 | 94.49 ± 1.44 | 25.12 | 0.57 |
| KC3-X1-PH10 | 111.20 | 87.23 ± 2.37 | 23.94 | −1.26 |
| KC3-X1-PH11 | 112.30 | 92.72 ± 3.40 | 27.20 | −3.23 |
| KC3-X1-PH12 | 107.80 | 88.82 ± 2.31 | 22.33 | −2.31 |
| KC3-X1-W | 143.20 | 14.13 ± 0.28 | −11.68 | −14.47 |
| KC3-X2-K | 107.80 | 95.35 ± 4.16 | 25.91 | −0.88 |
| KC3-OA | 126.70 | 92.26 ± 2.96 | 21.04 | −3.53 |

The formulations ranged in size from approximately 108 to 155 nm in diameter. The % entrapped mRNA ranged from 14-95% and LNPs exhibited a surface charge range at pH 5 from −12 to +27 mV and a range from −14 to +1 mV at pH 7.

TABLE 8H-2
Luciferase expression in MutuDC 1940 cells after
exposure to LNPs for 24 h at 0.3 μg/mL total
(encapsulated + non-encapsulated) mRNA concentration.

| Lipid | Luc activity (ng/10\hat{ }6 | ICL/KC3-OA |
|---|---|---|
| Composition | cells) ± stdev | activity |
| KC3-X2-R | 205.4 ± 23.4 | 1.06 |
| KC3-X2-K9 | 443.6 ± 43.4 | 2.29 |
| KC3-X1-PH10 | 270.4 ± 25.3 | 1.40 |
| KC3-X1-PH11 | 207.8 ± 15.3 | 1.07 |
| KC3-X1-PH12 | 346.0 ± 25.7 | 1.79 |
| KC3-X1-W | 1.3 ± 0.3 | 0.01 |
| KC3-X2-K | 412.8 ± 19.8 | 2.13 |
| KC3-OA | 193.5 ± 19.4 | 1.00 |

### Example 8H Evaluating PS-Targeted LNPs Comprising ICLs KC2-X1-S, KC3A-X1-S, KC3B-X1-S, KC3C-X1-S, KC3-X1-AA, KC3-X1-Z, KC4-X1-S and KC3-OA (Control)

The ICLs KC2-X1-S, KC3A-X1-S, KC3B-X1-S, KC3C-X1-S, KC3-X1-AA, KC3-X1-Z, KC4-X1-S and KC3-OA (control) were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8H-1
Physicochemical properties and characterization
of DPPS containing LNPs containing FFluc mRNA

| | Particle Size | E.E, %, | | |
|---|---|---|---|---|
| Lipid Composition | (nm) | Stdev | Zeta pH 5 | Zeta pH 7 |
| KC2-X1-S | 95.73 | 76.13 ± 4.78 | 14.4 | −12.0 |
| KC3A-X1-S | 133.10 | 97.94 ± 2.55 | 15.4 | −2.2 |
| KC3B-X1-S | 145.60 | 98.01 ± 3.17 | 15.0 | −0.8 |
| KC3C-X1-S | 162.30 | 88.04 ± 1.45 | 4.1 | 0.8 |
| KC3-X1-AA | 110.20 | 86.20 ± 2.05 | 15.4 | −8.0 |
| KC3-X1-Z | 115.00 | 85.96 ± 1.44 | 12.9 | −8.7 |
| KC4-X1-S | 134.80 | 94.03 ± 3.21 | 14.4 | −6.9 |
| KC3-OA | 128.90 | 90.11 ± 1.92 | 13.4 | −5.8 |

The formulations ranged in size from approximately 96 to 162.3 nm in diameter. The % entrapped mRNA ranged from 76-98% and LNPs exhibited a surface charge range at pH 5 from approximately +4 to +15 mV and a range from −12 to +1 mV at pH 7.

TABLE 8H-2
Luciferase expression in MutuDC 1940 cells after
exposure to LNPs for 24 h at 0.1 μg/mL total
(encapsulated + non-encapsulated) mRNA concentration.

| Lipid | Luc activity (ng/10\hat{ }6 | ICL/KC3-OA |
|---|---|---|
| Composition | cells) | activity |
| KC2-X1-S | 95.2 ± 7.0 | 0.71 |
| KC3A-X1-S | 6.8 ± 0.3 | 0.05 |
| KC3B-X1-S | 2.4 ± 0.1 | 0.02 |
| KC3C-X1-S | 5.0 ± 0.3 | 0.04 |
| KC3-X1-AA | 217.4 ± 18.1 | 1.62 |
| KC3-X1-Z | 161.6 ± 19.9 | 1.20 |
| KC4-X1-S | 109.4 ± 6.7 | 0.81 |
| KC3-OA | 134.4 ± 3.1 | 1.00 |

TABLE 8H-3
Luciferase expression in MutuDC 1940 cells after
exposure to LNPs for 24 h at 0.3 μg/mL total
(encapsulated + non-encapsulated) mRNA concentration.

| | Luc activity (ng/10\hat{ }6 | ICL/KC3-OA |
|---|---|---|
| Lipid Composition | cells) ± stdev | activity |
| KC2-X1-S | 244.0 ± 11.6 | 1.00 |
| KC3A-X1-S | 34.1 ± 4.2 | 0.14 |
| KC3B-X1-S | 12.6 ± 2.1 | 0.05 |
| KC3C-X1-S | 16.7 ± 1.8 | 0.07 |
| KC3-X1-AA | 487.7 ± 31.8 | 1.99 |
| KC3-X1-Z | 379.8 ± 30.0 | 1.55 |
| KC4-X1-S | 280.0 ± 7.6 | 1.14 |
| KC3-OA | 244.9 ± 16.3 | 1.00 |

### Example 81 Evaluating PS-Targeted LNPs Comprising ICLs KC3-X2-PH8, KC3-X2-PH9, KC3-X2-R₉, KC4-X1-PH5, KC4-X1-PH6, KC3-X1-W, and KC3-OA

The ICLs KC3-X2-PH8, KC3-X2-PH9, KC3-X2-R9, KC4-X1-PH5, KC4-X1-PH6, KC3-X1-W, and KC3-OA were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8I-1
Physicochemical properties and characterization
of DPPS containing LNPs containing FFluc mRNA

| | Particle Size | E.E, %, | | |
|---|---|---|---|---|
| Lipid Composition | (nm) | Stdev | Zeta pH 5 | Zeta pH 7 |
| KC2-X1-PH6 | 85.57 | 86.42 ± 1.51 | 7.86 | −7.23 |
| KC3-X2-PH8 | 115.90 | 86.70 ± 2.44 | 18.96 | −2.91 |
| KC3-X2-PH9 | 109.80 | 86.38 ± 1.28 | 18.40 | −2.52 |
| KC3-X2-R9 | 145.60 | 88.94 ± 1.79 | 7.72 | −1.75 |
| KC4-X1-PH5 | 117.10 | 96.81 ± 6.01 | 17.10 | −3.15 |
| KC4-X1-PH6 | 110.80 | 96.26 ± 3.28 | 22.87 | −3.60 |
| KC3-X1-W | n/a | n/a | n/a | n/a |
| KC3-OA | 116.80 | 89.39 ± 3.40 | 13.26 | −1.12 |

The formulations ranged in size from approximately 86 to 117 nm in diameter. The % entrapped mRNA ranged from 86-97% and LNPs exhibited a surface charge range at pH 5 from approximately +8 to +23 mV and a range from −7 to +1 mV at pH 7.

TABLE 8I-2
Luciferase expression in MutuDC 1940 cells after
exposure to LNPs for 24 h at 0.3 μg/mL total
(encapsulated + non-encapsulated) mRNA concentration.

| | Luc activity | ICL/KC3-OA |
|---|---|---|
| Lipid Composition | (ng/10\hat{ }6 cells) ± stdev | activity |
| KC2-X1-PH6 | 224.2 ± 14.2 | 0.95 |
| KC3-X2-PH8 | 301.6 ± 30.1 | 1.28 |
| KC3-X2-PH9 | 367.6 ± 18.0 | 1.56 |
| KC4-X1-PH5 | 289.1 ± 23.9 | 1.05 |
| KC4-X1-PH6 | 231.5 ± 19.9 | 0.98 |
| KC3-OA | 236.0 ± 22.2 | 1.00 |

### Example 8J Evaluating PS-Targeted LNPs Comprising ICLs KC3-X2-PH6, KC3-X2-PH7, KC3-X2-S9

The ICLs KC3-X2-PH6, KC3-X2-PH7, KC3-X2-S9 were separately formulated in LNPs comprising of ICL/DSPC/DPPS-NH4/Chol/PEG-DMG (52/5/5/36.5/1.5 mol %) and at an N/P ratio of 5.25 encapsulating firefly luciferase mRNA (FFluc). The LNPs were used to evaluate ICL activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8J-1
Physicochemical properties and characterization
of DPPS containing LNPs containing FFluc mRNA

| | Particle Size | E.E, %, | | |
|---|---|---|---|---|
| Lipid Composition | (nm) | Stdev | Zeta pH 5 | Zeta pH 7 |
| KC3-X2-PH6 | 87.16 | 93.95 ± 0.20 | 19.96 | 1.03 |
| KC3-X2-PH7 | 93.82 | 90.40 ± 0.24 | 18.73 | −1.74 |
| KC3-X2-S9 | 96.66 | 87.55 ± 0.26 | 16.94 | −3.60 |

The formulations ranged in size from approximately 87 to 97 nm in diameter. The % entrapped mRNA ranged from 87-94% and LNPs exhibited a surface charge range at pH 5 from approximately +17 to +19 mV and a range from −4 to +1 mV at pH 7.

TABLE 8J-2
Luciferase expression in MutuDC 1940 cells after exposure to LNPs
for 24 h at 0.3 μg/mL total (encapsulated + non-encapsulated)
mRNA concentration. A frozen KC3-OA LNP formulation described
in Example 33 was used in the luciferase assay as a control.

| | Luc activity (ng/10\hat{ }6 cells) ± | ICL/KC3-OA |
|---|---|---|
| Lipid Composition | stdev | activity |
| KC3-X2-PH6 | 140.0 ± 11.8 | 0.36 |
| KC3-X2-PH7 | 255.5 ± 13.6 | 0.65 |
| KC3-X2-S9 | 463.2 ± 9.4 | 1.18 |
| KC3-OA (frozen | 392.83 ± 33.7 | 1.0 |
| control) | | |

### Example 8K. Evaluating PS-Containing LNPs Comprising KC3-OA or KC3-X1-PH5 or KC3-X1-PH12 or KC3-X1-AA or KC3-X2-K9

PS-containing LNPs containing ICLs were prepared at varying mol % encapsulating the stabilized form of H5N1 A/Texas/37/2024 mRNA which was used to evaluate the effect of ICL content on immunogenicity in BALB/c mice.

LNPs were prepared comprising of either KC3-OA or KC3-X1-PH5 or KC3-X1-PH12 or KC3-X1-AA or KC3-X2-K9 with the formulation composition ICL/DSPC/DPPS-NH₄/Chol/PEG-DMG (52/5/5/36.5/1.5 mol %) at N/P ratio 5.25 or KC3-X1-PH5/DSPC/DPPS-NH₄/Chol/PEG-DMG (48/5/5/40.5/1.5 mol %) at N/P ratio 5.25 or KC3-X1-PH5/DSPC/DPPS-NH₄/Chol/PEG-DMG (56/5/5/32.5/1.5 mol %) at N/P ratio 5.25.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8K-1
Physicochemical properties and characterization of
LNPs containing H5N1 A/Texas/37/2024 mRNA mRNA.

| | Mol % | Particle | E.E, %, | Zeta pH | Zeta pH |
|---|---|---|---|---|---|
| Composition | ICL | Size (nm) | Stdev | 5 | 7 |
| KC3-X1-PH5 | 48 | 101.50 | 92.9 ± 5.0 | 16.7 | −3.0 |
| KC3-X1-PH5 | 50 | 107.70 | 93.1 ± 3.8 | 13.0 | −3.1 |
| KC3-X1-PH5 | 52 | 119.90 | 91.2 ± 3.1 | 12.6 | −4.2 |
| KC3-OA | 52 | 106.00 | 89.6 ± 2.2 | 11.5 | −4.5 |
| KC3-X1-AA | 52 | 104.30 | 89.5 ± 2.2 | 11.3 | −4.4 |
| KC3-X2-K9 | 52 | 124.00 | 91.9 ± 3.2 | 14.0 | −2.8 |
| KC3-X1-PH12 | 52 | 102.60 | 85.4 ± 4.8 | 13.1 | −3.9 |

The formulations ranged in size from approximately 102 to 124 nm in diameter. The % entrapped mRNA ranged from 85-93% and LNPs exhibited positive surface charge at pH 5 (~+11 to +17 mV) and a negative charge at pH 7 (−3 to −5 mV).

### Example 8L. Evaluating PS-Containing LNPs Comprising KC3-X1-PH12

LNPs comprising 48, 52, 54, 56, 60 or 64 mol % KC3-X1-PH12 with the composition KC3-X1-PH12/DSPC/DPPS/Chol/PEG-DMG (x/5/5/(88.5-x)/1.5 mol %) and an N/P ratio of 4.5 encapsulating firefly luciferase mRNA (FFluc, SEQ ID NO:3) were prepared, where x represents the mol % ionizable lipid. The LNPs were used to evaluate the effect of altering the mol % of KC3-X1-PH12 on in vitro luciferase activity in vitro.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 8L-1
Physicochemical properties and characterization of DPPS
containing KC3-X1-PH12 LNPs containing FFluc mRNA

| | Particle Size | E.E, %, | | |
|---|---|---|---|---|
| Mol % | (nm) | Stdev | Zeta pH 5 | Zeta pH 7 |
| 48 | 101.80 | 87.02 ± 0.76 | 18.68 | −1.84 |
| 52 | 104.20 | 87.42 ± 0.54 | 16.04 | −2.36 |
| 56 | 109.40 | 88.65 ± 0.45 | 15.34 | −3.38 |
| 60 | 111.70 | 88.76 ± 0.52 | 18.70 | −3.34 |
| 64 | 119.30 | 87.06 ± 0.67 | 13.73 | −4.40 |

The formulations ranged in size from approximately 104 to 112 nm in diameter. The % entrapped mRNA ranged from % and LNPs exhibited a surface charge range at pH 5 from approximately +14 to +19 mV and a range from −4 to −2 mV at pH 7.

TABLE 8L-2
Luciferase expression in MutuDC 1940 cells after exposure to LNPs
for 24 h at 0.3 μg/mL total (encapsulated + non-encapsulated)
mRNA concentration. A frozen KC3-OA LNP formulation described
in Example 34 was used in the luciferase assay as a control.

| | Luc activity (ng/10\hat{ }6 | Luc Activity KC3-X1- |
|---|---|---|
| Mol % | cells) ± stdev | PH12/KC3OA |
| 48 | 421.7 ± 21.8 | 1.07 |
| 52 | 506.5 ± 24.8 | 1.29 |
| 56 | 662.4 ± 44.3 | 1.69 |
| 60 | 592.2 ± 27.8 | 1.51 |
| 64 | 417.8 ± 25.5 | 1.06 |

### Example 9. Preparation and Analysis of LNPs Containing Different ICLs at Different N/P Ratios Encapsulating FFluc mRNA Used to Evaluate Activity In Vitro

### Example 9A. Preparation and Analysis of LNPs Containing KC3-X1-PH5

LNPs were prepared comprising of KC3-X1-PH5/DSPC/DPPS-NH₄/Chol/PEG-DMG (52/5/5/36.5/1.5 mol %) at varying N/P ratios containing the reporter mRNA FFluc and evaluated in vitro for luciferase activity.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 9A-1
Physicochemical properties and characterization of KC3-X1-
PH5 LNPs containing FFluc mRNA at different N/P ratios.

| | Particle Size | E.E, %, | Zeta pH | Zeta pH |
|---|---|---|---|---|
| N/P | (nm) | Stdev | 5 | 7 |
| 4.0 | 136.70 | 88.08 ± 6.08 | 15.86 | −4.12 |
| 4.25 | 132.40 | 90.94 ± 11.01 | 16.82 | −3.13 |
| 5.0 | 122.80 | 94.04 ± 8.48 | 20.74 | −6.28 |
| 5.5 | 112.70 | 94.49 ± 7.20 | 17.13 | −6.20 |
| 6.0 | 118.00 | 94.42 ± 3.94 | 19.12 | −5.79 |
| 6.5 | 118.10 | 94.61 ± 5.49 | 18.24 | −5.32 |

TABLE 9A-2
Luciferase expression (ng/million cells ± stdev) in MutuDC
1940 cells after exposure to LNPs for 24 h at 0.3 μg/mL
total (encapsulated + non-encapsulated) mRNA concentration.

| N/P | Luc activity | Luc activity Ratio |
|---|---|---|
| 5.0 | 162.71 ± 9.35 | 1.00 |
| 5.25 | 179.79 ± 6.06 | 1.10 |
| 5.5 | 199.65 ± 7.81 | 1.23 |
| 5.75 | 193.20 ± 6.55 | 1.19 |
| 6.0 | 183.62 ± 3.25 | 1.13 |
| 6.5 | 175.73 ± 11.98 | 1.08 |

TABLE 9A-3
Luciferase expression (ng/million cells ± stdev) in MutuDC
1940 cells after exposure to LNPs for 24 h at 0.1 μg/mL
total (encapsulated + non-encapsulated) mRNA concentration.

| N/P | Luc activity | Luc activity Ratio |
|---|---|---|
| 5.0 | 56.85 ± 4.36 | 1.00 |
| 5.25 | 67.21 ± 4.47 | 1.18 |
| 5.5 | 73.24 ± 2.82 | 1.29 |
| 5.75 | 73.09 ± 2.45 | 1.29 |
| 6.0 | 87.62 ± 2.69 | 1.54 |
| 6.5 | 79.64 ± 6.98 | 1.40 |

### Example 9B. Preparation and Analysis of LNPs Containing KC3-X1-AA and KC3-X1-PH6

LNPs were prepared comprising of ICL/DSPC/DPPS-NH₄/Chol/PEG-DMG (52/5/5/36.5/1.5 mol %) at varying N/P ratios containing the reporter mRNA FFluc and evaluated in vitro for luciferase activity.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 9B-1
Physicochemical properties and characterization
of KC3-X1-PH6 and KC3-X1-AA LNPs containing
FFluc mRNA at different N/P ratios.

| | | Particle | E.E, %, | Zeta pH | Zeta pH |
|---|---|---|---|---|---|
| ICL | N/P | Size (nm) | Stdev | 5 | 7 |
| KC3-X1-PH5 | 4.0 | 114.60 | 95.63 ± 7.04 | 16.12 | −7.35 |
| KC3-X1-PH5 | 4.25 | 114.60 | 94.76 ± 7.04 | 18.28 | −3.93 |
| KC3-X1-PH5 | 5.0 | 116.10 | 95.76 ± 7.04 | 21.06 | −3.50 |
| KC3-X1-PH5 | 5.5 | 114.40 | 95.42 ± 7.04 | 22.90 | −2.67 |
| KC3-X1-PH5 | 6.0 | 111.90 | 95.00 ± 7.04 | 22.09 | −2.91 |
| KC3-X1-PH5 | 6.5 | 114.50 | 95.61 ± 7.04 | 26.79 | −3.46 |
| KC3-X1-AA | 4.0 | 113.20 | 93.08 ± 7.04 | 15.84 | −5.49 |
| KC3-X1-AA | 4.25 | 107.00 | 92.07 ± 7.04 | 16.66 | −6.37 |
| KC3-X1-AA | 5.0 | 111.00 | 92.19 ± 7.04 | 18.10 | −6.15 |
| KC3-X1-AA | 5.5 | 117.40 | 90.76 ± 7.04 | 20.56 | −6.33 |
| KC3-X1-AA | 6.0 | 115.90 | 91.17 ± 7.04 | 21.09 | −7.59 |
| KC3-X1-AA | 6.5 | 119.20 | 91.44 ± 7.04 | 23.97 | −6.21 |

TABLE 9B-2
Luciferase expression (ng/million cells ± stdev) in MutuDC
1940 cells after exposure to LNPs for 24 h at 0.3 μg/mL
total (encapsulated + non-encapsulated) mRNA concentration.

| ICL | N/P | Luc Activity | Luc Ratio |
|---|---|---|---|
| KC3-X1-PH5 | 4.0 | 273.3 ± 19.8 | 1.00 |
| KC3-X1-PH5 | 4.25 | 284.4 ± 13.4 | 1.04 |
| KC3-X1-PH5 | 5.0 | 295.3 ± 9.3 | 1.08 |
| KC3-X1-PH5 | 5.5 | 287. ± 12.4 | 1.05 |
| KC3-X1-PH5 | 6.0 | 302.3 ± 13.1 | 1.11 |
| KC3-X1-PH5 | 6.5 | 293.5 ± 11.8 | 1.07 |
| KC3-X1-AA | 4.0 | 389.5 ± 32.5 | 1.43 |
| KC3-X1-AA | 4.25 | 404.4 ± 16.2 | 1.48 |
| KC3-X1-AA | 5.0 | 380.1 ± 31.6 | 1.39 |
| KC3-X1-AA | 5.5 | 351.1 ± 20.7 | 1.28 |
| KC3-X1-AA | 6.0 | 354.2 ± 14.4 | 1.30 |
| KC3-X1-AA | 6.5 | 355.1 ± 20.4 | 1.30 |

TABLE 9B-3
Luciferase expression (ng/million cells ± stdev) in MutuDC
1940 cells after exposure to LNPs for 24 h at 0.1 μg/mL
total (encapsulated + non-encapsulated) mRNA concentration.

| ICL | N/P | Luc Activity | Luc Ratio |
|---|---|---|---|
| KC3-X1-PH5 | 4.0 | 103.7 ± 9.9 | 1.00 |
| KC3-X1-PH5 | 4.25 | 118.9 ± 4.4 | 1.15 |
| KC3-X1-PH5 | 5.0 | 138.9 ± 6.0 | 1.34 |
| KC3-X1-PH5 | 5.5 | 142.8 ± 6.9 | 1.38 |
| KC3-X1-PH5 | 6.0 | 146.6 ± 4.8 | 1.41 |
| KC3-X1-PH5 | 6.5 | 157.8 ± 10.7 | 1.52 |
| KC3-X1-AA | 4.0 | 174.1 ± 6.5 | 1.68 |
| KC3-X1-AA | 4.25 | 196.3 ± 14.8 | 1.89 |
| KC3-X1-AA | 5.0 | 185.9 ± 17.4 | 1.79 |
| KC3-X1-AA | 5.5 | 171.0 ± 10.1 | 1.65 |
| KC3-X1-AA | 6.0 | 179.3 ± 9.1 | 1.73 |
| KC3-X1-AA | 6.5 | 182.6 ± 13.4 | 1.76 |

### Example 9C. Preparation and Analysis of Lnps Containing Kc3-X1-Ph9

LNPs were prepared comprising of KC3-X2-PH9/DSPC/DPPS-NH₄/Chol/PEG-DMG (52/5/5/36.5/1.5 mol %) at varying N/P ratios containing the reporter mRNA FFluc and evaluated in vitro for luciferase activity.

Particle size refers to the Z-Average particle size after freezing and thawing. Zeta refers to zeta potential measurements at either pH 5 or pH 7 in units of mV. mRNA encapsulation efficiency is referred to as (E.E,%).

TABLE 9C-1
Physicochemical properties and characterization of KC3-X2-K9
LNPs containing FFluc mRNA at different N/P ratios. Some
formulations were prepared on more than one occasion.

| | Particle | E.E, %, | | |
|---|---|---|---|---|
| N/P | Size (nm) | Stdev | Zeta pH 5 | Zeta pH 7 |
| 5.0 | 133.1 | 92.33 ± 3.98 | 20.00 | 1.87 |
| 5.25 | 128.90 | 92.51 ± 2.57 | 18.19 | 0.06 |
| 5.5 | 135.10 | 92.88 ± 6.97 | 16.26 | 0.34 |
| 5.75 | 128.5 | 93.12 ± 5.62 | 22.58 | −0.22 |
| 6.0 | 140.30 | 92.50 ± 3.23 | 25.89 | −0.19 |

TABLE 9C-2
Luciferase expression (ng/million cells ± stdev) in MutuDC
1940 cells after exposure to LNPs for 24 h at 0.3 μg/mL
total (encapsulated + non-encapsulated) mRNA concentration.

| N/P | Luc activity | Luc ratio |
|---|---|---|
| 5.0 | 600.66 ± 47.09 | 1.00 |
| 5.25 | 669.08 ± 62.82 | 1.11 |
| 5.5 | 652.95 ± 45.18 | 1.09 |
| 5.75 | 642.09 ± 70.23 | 1.07 |
| 6.0 | 681.19 ± 80.09 | 1.13 |

### Example 9D. Preparation and Analysis of LNPs Containing KC3-X1-PH12

LNPs were prepared comprising of KC3-X1-PH12/DSPC/DPPS-NH₄/Chol/PEG-DMG (52/5/5/36.5/1.5 mol %) at varying N/P ratios containing the reporter mRNA FFluc and evaluated in vitro for luciferase activity.

Particle size refers to the Z-Average particle size after freezing and thawing. Zeta refers to zeta potential measurements at either pH 5 or pH 7 in units of mV. mRNA encapsulation efficiency is referred to as (E. E, %).

TABLE 9D-1
Physicochemical properties and characterization of KC3-X1-
PH12 LNPs containing FFluc mRNA at different N/P ratios.
Some formulations were prepared on more than one occasion.

| | Particle | E.E, %, | Zeta pH | Zeta pH |
|---|---|---|---|---|
| N/P | Size (nm) | Stdev | 5 | 7 |
| 1.0 | 129.00 | 16.07 ± 0.48 | −0.08 | −27.50 |
| 2.0 | 151.20 | 56.87 ± 2.77 | 0.29 | −6.67 |
| 3.0 | 109.50 | 76.03 ± 2.39 | 6.80 | −1.54 |
| 3.25 | 110.20 | 82.23 ± 2.17 | 10.95 | −1.50 |
| 3.5 | 109.70 | 89.15 ± 3.14 | 12.50 | −2.95 |
| 3.75 | 111.30 | 93.00 ± 2.93 | 12.99 | −3.60 |
| 4.0 | 107.90 | 92.08 ± 1.99 | 15.52 | −4.01 |
| 4.25 | 107.40 | 92.27 ± 3.66 | 15.22 | −5.62 |
| 4.5 | 107.30 | 92.25 ± 2.68 | 14.96 | −5.37 |
| 4.75 | 110.40 | 92.06 ± 3.88 | 17.68 | −8.92 |
| 5.0 | 108.80 | 92.53 ± 2.02 | 18.66 | −6.93 |
| 4.0 | 108.30 | 89.58 ± 2.13 | 7.347 | −5.356 |
| 4.25 | 104.10 | 88.59 ± 3.19 | 7.788 | −6.262 |
| 4.5 | 105.30 | 89.21 ± 3.33 | 8.238 | −6.534 |
| 4.75 | 110.20 | 89.29 ± 2.45 | 11.34 | −4.663 |
| 5.0 | 105.50 | 88.37 ± 2.67 | 12.34 | −6.036 |
| 5.0 | 114.60 | 89.71 ± 3.51 | 23.56 | −0.36 |
| 5.25 | 113.40 | 91.80 ± 5.23 | 16.56 | −2.33 |
| 5.50 | 108.60 | 89.57 ± 2.81 | 17.31 | −2.29 |
| 5.75 | 109.30 | 90.33 ± 1.74 | 24.24 | −1.67 |
| 6.00 | 110.40 | 91.54 ± 4.31 | 19.47 | −3.63 |

TABLE 9D-2
Luciferase expression (ng/million cells ± stdev) in MutuDC
1940 cells after exposure to LNPs for 24 h at 0.3 μg/mL
total (encapsulated + non-encapsulated) mRNA concentration.

| N/P | Luc activity | Luc Ratio |
|---|---|---|
| 1.0 | 0.90 ± 0.13 | 1.00 |
| 2.0 | 1.89 ± 1.33 | 2.10 |
| 3.0 | 139.52 ± 5.09 | 155.02 |
| 3.25 | 252.30 ± 11.47 | 280.33 |
| 3.5 | 357.14 ± 11.79 | 396.82 |
| 3.75 | 439.23 ± 27.62 | 488.03 |
| 4.0 | 435.03 ± 38.70 | 483.37 |
| 4.25 | 441.10 ± 38.06 | 490.11 |
| 4.5 | 469.42 ± 37.90 | 521.58 |
| 4.75 | 400.85 ± 20.80 | 445.39 |
| 5.0 | 400.11 ± 36.32 | 444.57 |
| 4.0 | 536.19 ± 25.21 | 595.77 |
| 4.25 | 494.61 ± 42.91 | 549.57 |
| 4.5 | 471.19 ± 63.12 | 523.54 |
| 4.75 | 476.97 ± 60.50 | 529.97 |
| 5.0 | 498.15 ± 50.44 | 553.50 |
| 5.0 | 457.75 ± 28.64 | 508.61 |
| 5.25 | 429.82 ± 32.22 | 477.58 |
| 5.50 | 390.98 ± 19.98 | 434.42 |
| 5.75 | 379.55 ± 29.30 | 421.72 |
| 6.00 | 372.24 ± 26.92 | 413.60 |

### Example 9E Preparation and Analysis of LNPs Containing KC3-X1-PH12

The mRNA encoding Ffluc-mRNA was formulated separately into LNPs comprising an ICL/DSPC/DPPS-NH4/Chol/PEG-DMG where the ICL mol % was varied from 48, 52, 56, DSPC and DPPS-NH4 present at 5 mol % each and PEG-DMG at 1.5 mol % and the remainder lipid comprising of cholesterol, each set formulated at an N/P ratio of 4.5 or 5.25.

The LNP formulation and the KC3-based LNPs were formulated using a ChipShop 187 microfluidic mixer chip at a flow rate of 12 mL/min and analyzed as in Example 4.

Particle size refers to the Z-Average particle size after freezing and thawing. Zeta refers to zeta potential measurements at either pH 5 or pH 7 in units of mV. mRNA encapsulation efficiency after freezing & thawing is referred to as (E.E,%).

TABLE 9E-1
Physicochemical properties and characterization of LNPs containing Ffluc mRNA
comparing different ICLs at varying mol % ICLs and N/P ratios

| | | | | | Zeta | |
|---|---|---|---|---|---|---|
| ICL | N/P | mol % | Z-Average | % EE | pH5 | Zeta pH 7 |
| KC3-X1-PH12 | 4.5 | 48 | 91.40 | 95.26 ± 0.25 | 16.33 | −2.35 |
| | | 52 | 107.20 | 92.28 ± 0.24 | 14.97 | −1.78 |
| | | 56 | 113.00 | 85.63 ± 0.60 | 12.19 | −3.24 |
| | 5.25 | 48 | 86.56 | 95.74 ± 0.24 | 17.05 | −0.50 |
| | | 52 | 99.79 | 94.32 ± 0.26 | 16.40 | −2.11 |
| | | 56 | 94.67 | 95.58 ± 0.25 | 15.76 | −2.25 |
| KC3-X2-PH10 | 4.5 | 48 | 86.23 | 96.25 ± 0.16 | 18.84 | −2.39 |
| | | 52 | 89.65 | 96.17 ± 0.17 | 11.75 | −2.68 |
| | | 56 | 91.85 | 96.57 ± 0.0.19 | 14.42 | −0.14 |
| | 5.25 | 48 | 82.73 | 96.89 ± 0.17 | 17.00 | −0.21 |
| | | 52 | 88.06 | 96.82 ± 0.13 | 13.73 | −1.27 |
| | | 56 | 90.05 | 96.95 ± 0.25 | 11.11 | −2.51 |
| KC3-X2-PH11 | 4.5 | 48 | 87.75 | 94.73 ± 0.23 | 18.67 | −0.77 |
| | | 52 | 101.50 | 92.58 ± 0.32 | 19.18 | −1.11 |
| | | 56 | 99.80 | 93.69 ± 0.07 | 19.11 | −0.71 |
| | 5.25 | 48 | 84.73 | 95.67 ± 0.21 | 18.93 | −1.57 |
| | | 52 | 89.22 | 95.24 ± 0.22 | 19.26 | −1.63 |
| | | 56 | 95.29 | 95.23 ± 0.06 | 13.89 | −1.90 |
| KC3-X2-PH12 | 4.5 | 48 | 94.28 | 93.48 ± 0.19 | 13.25 | −1.28 |
| | | 52 | 102.60 | 92.35 ± 0.49 | 15.55 | −2.25 |
| | | 56 | 114.30 | 84.98 ± 0.50 | 14.50 | −2.06 |
| | 5.25 | 48 | 86.14 | 95.03 ± 0.06 | 15.12 | −2.53 |
| | | 52 | 93.42 | 94.14 ± 0.19 | 16.91 | −0.74 |
| | | 56 | 107.50 | 90.76 ± 0.25 | 19.53 | −0.50 |
| KC3-X1-PH5 | 4.5 | 48 | 83.72 | 97.46 ± 0.18 | 15.52 | −2.24 |
| | | 52 | 86.99 | 97.25 ± 0.13 | 17.70 | −4.66 |
| | | 56 | 94.12 | 96.92 ± 0.15 | 18.98 | −4.29 |
| | 5.25 | 48 | 79.66 | 97.57 ± 0.18 | 14.69 | −3.35 |
| | | 52 | 85.82 | 97.37 ± 0.11 | 17.26 | −5.12 |
| | | 56 | 88.82 | 97.47 ± 0.06 | 12.99 | −2.99 |

The formulations ranged in size from approximately 80 to 114 nm in diameter The % entrapped mRNA ranged from 85-9800 and LNPs exhibited positive surface charge at pH 5 ~+11-19 and a negative charge at pH 7-5-0 mV.

The activity of the LNPs was evaluated by transfection MutuDC1940 cells at 0.3 ug/mL mRNA concentration for 24 h as described in Example 6A.

TABLE 9E-2
In vitro activity of LNPs containing Ffluc mRNA comparing
different ICLs at varying mol % ICLs and N/P ratios

| ICL | N/P | mol % | Luc Activity ± stdev |
|---|---|---|---|
| AKG-KC3-X1-PH12 | 4.5 | 48 | 447.43 ± 14.69 |
| | | 52 | 499.75 ± 52.42 |
| | | 56 | 511.05 ± 33.44 |
| | 5.25 | 48 | 415.31 ± 28.10 |
| | | 52 | 482.38 ± 33.56 |
| | | 56 | 353.39 ± 11.04 |
| AKG-KC3-X2-PH10 | 4.5 | 48 | 271.38 ± 22.23 |
| | | 52 | 292.41 ± 18.60 |
| | | 56 | 280.18 ± 21.39 |
| | 5.25 | 48 | 266.84 ± 22.64 |
| | | 52 | 288.96 ± 23.43 |
| | | 56 | 331.37 ± 27.45 |
| KC3-X2-PH11 | 4.5 | 48 | 481.04 ± 23.42 |
| | | 52 | 517.26 ± 51.10 |
| | | 56 | 480.88 ± 54.88 |
| | 5.25 | 48 | 493.79 ± 38.95 |
| | | 52 | 428.00 ± 33.76 |
| | | 56 | 564.76 ± 55.93 |
| KC3-X2-PH12 | 4.5 | 48 | 639.25 ± 53.28 |
| | | 52 | 698.89 ± 59.32 |
| | | 56 | 835.59 ± 40.02 |
| | 5.25 | 48 | 509.32 ± 62.64 |
| | | 52 | 624.24 ± 75.30 |
| | | 56 | 812.14 ± 58.59 |
| AKG-KC3-X1-PH5 | 4.5 | 48 | 305.63 ± 23.66 |
| | | 52 | 315.05 ± 33.64 |
| | | 56 | 274.47 ± 33.93 |
| | 5.25 | 48 | 295.25 ± 22.55 |
| | | 52 | 322.90 ± 25.60 |
| | | 56 | 269.68 ± 34.76 |

It was found that the LNPs with the highest transfection activity when compared across every mol % and N/P ratio tested contained the ICL KC3-X1-PH12.

Immunogenicity of LNP Compositions Comprising mRNA Encoding Immunogenic Proteins

LNP compositions encapsulating mRNA encoding various proteins were prepared as described below, and the immunogenicity of the compositions was assessed in vivo. The following sequences are referenced in the examples below. SEQ ID NO:407 is a second gen LAMP-1 protein sequence (SEQ ID NO: 407). SEQ ID NO:408 is a second gen LAMP-1 mRNA sequence. Includes 5′ HBB UTR and 3′ HBB UTR (SEQ ID NO: 408). SEQ ID NO:5 is a H5 protein sequence (SEQ ID NO: 5). SEQ ID NO:6 is a H5 mRNA sequence that includes 5′ HBB UTR and 3′ HBB UTR (SEQ ID NO: 6). SEQ ID NO: 15 is a H5 protein sequence. SEQ ID NO: 16 is a H5 mRNA sequence that includes 5′ HBB UTR and 3′ HBB UTR (SEQ ID NO: 16). SEQ ID NO:417 is a third gen LAMP-1 protein sequence (Esx fusions) (SEQ ID NO: 417). SEQ ID NO:418 is a third gen LAMP-1 mRNA sequence. Includes 5′ HBB UTR and 3′ HBB UTR (SEQ ID NO: 418).

### Example 10. Preparation and Analysis of LNPs Containing Biodegradable ICLs Used to Evaluate Antigenic Responses in Mice Using an mRNA Encoding a Synthetic Tuberculosis Antigen

### Example 10A. Preparation and Analysis of LNPs Containing Biodegradable ICLs Used to Evaluate T-Cell Responses in Mice Using an mRNA Encoding a Synthetic Tuberculosis Antigen

The purpose of this study was to compare the immunogenicity of mRNA-LNPs consisting of KC3-OA or panel of novel biodegradable ICLs. To track B-cell and T-cell responses, we used an mRNA encoding a Mycobacterium tuberculosis synthetic vaccine antigen (SEQ ID NOs: 407 and 408). The ICLs KC3-X1-PH5, KC3-X1-PH6, KC3-X1-PH8, KC3-X1-PH12, KC3-X1-S, KC3-X2-K9, KC4-X1-H and KC3-OA were separately formulated in LNPs comprising of ICL/DSPC/DPPS-NH₄/Chol/PEG-DMG (52/5/5/36.5/1.5 mol %) with VRN905 mRNA (a second generation mRNA encoding a synthetic TB antigen) at an N/P ratio of 5.25. Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 112 to 124 nm in diameter. The % entrapped mRNA ranged from 92-96% and LNPs exhibited positive surface charge at pH 5 (~+18 to +25 mV) and a negative charge at pH 7 (−3 to −7 mV).

TABLE 10A-1
Physicochemical properties and characterization
of LNPs containing VRN905 mRNA.

| | Particle Size | E.E, %, | Zeta pH | Zeta pH |
|---|---|---|---|---|
| Lipid Composition | (nm) | Stdev | 5 | 7 |
| KC3-OA | 122.00 | 93.8 ± 1.9 | 19.96 | −3.13 |
| KC3-X1-PH5 | 118.30 | 95.9 ± 11.1 | 24.53 | −6.02 |
| KC3-X1-PH6 | 116.40 | 95.0 ± 8.12 | 22.85 | −5.96 |
| KC3-X1-PH8 | 115.90 | 92.5 ± 5.8 | 22.24 | −6.69 |
| KC3-X1-PH12 | 115.70 | 94.6 ± 4.6 | 23.30 | −4.47 |
| KC3-X1-S | 118.50 | 92.0 ± 5.4 | 18.42 | −6.45 |
| KC3-X2-K9 | 135.20 | 91.7 ± 1.2 | 21.17 | −4.14 |
| KC4-X1-H | 145.20 | 92.2 ± 2.7 | 22.41 | −3.55 |

To examine how effective these ICLs are at encapsulating mRNA and transfecting cells in vivo, CB6F1 hybrid mice were immunized i.m. in the caudal thigh with 1 or 0.3 ug mRNA and given a homologous boost on study day (SD) 32 (FIG. 10A). Splenic T cell responses were analyzed 8 days post-boost by stimulating cells with pools of 15mer peptides overlapping by 11 residues in the presence of the golgi inhibitor brefeldin A. Cells were stained with fluorescently-tagged antibodies and analyzed by flow cytometry for intracellular IFN-g, TNF-α and IL-2.

The magnitudes of the CD4 T cell (FIG. 10B) and CD8 T cell (FIG. 10C) TB-specific responses were generally comparable between LNP formulations at both the 1 and 0.3 ug dose, but there were some formulations that elicited smaller responses. For the CD4 responses, the ICLs KC3-X1-PH8 and KC3-X1-S were inferior relative to other formulations, most notable KC3-X1-PH12. For the CD8 responses, KC3-X1-PH8 trended smaller relative to the other formulations, and at the 0.3 ug dose, KC4-X1-H was inferior to most of the other ICLs. In conclusion, all of these mRNA-LNPs formulated with these biodegradable ICLs were capable of eliciting antigen-specific T cell immunity at the doses tested. All except for KC3-X1-PH8 and KC3-X1-S performed comparably to KC3-OA.

SEQ ID NO: 407 is a polypeptide sequence consisting of the human LAMP-1 N-terminal signal peptide, a collection of covalently linked M. tuberculosis proteins, and the C-terminal human LAMP-1 terminal domain. SEQ ID NO: 408 is a codon optimized RNA sequence corresponding to the polypeptide in SEQ ID NO. 407. The ORF is flanked by HBB UTRs.

### Example 10B. Preparation and Analysis of LNPs Containing Biodegradable ICLs Used to Evaluate T Cell Responses in Mice Using an mRNA Encoding a Synthetic Tuberculosis Antigen

The purpose of this study was to compare the immunogenicity of mRNA-LNPs consisting of KC3-OA or panel of novel biodegradable ICLs. To track T-cell responses, we used an mRNA encoding a Mycobacterium tuberculosis synthetic antigen (SEQ ID NOs: 417 and 418). The ICLs KC3-X1-H, KC3-X1-K, KC2-X1-PH5, KC3-X1-PH5, KC3-X1-PH4, KC3-X1-PH1 were separately formulated in LNPs comprising of ICL/DSPC/DPPS-NH4/Chol/PEG-DMG (52/5/5/36.5/1.5 mol %) and KC3-OA LNPs comprising (48/5/5/40.5/1.5 mol %) at an N/P ratio of 5.25 and were used to evaluate T cell responses in mice. Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 93 to 137 nm in diameter. The % entrapped mRNA ranged from 80-98% and LNPs exhibited positive surface charge at pH 5 (~+22 to +28 mV) and a negative charge at pH 7 (−1.6 to −12.5 mV).

TABLE 10B-1
Physicochemical properties and characterization of LNPs
containing an M. tuberculosis vaccine antigen mRNA.

| Lipid | Particle | E.E, %, | | |
|---|---|---|---|---|
| Composition | Size (nm) | Stdev | Zeta pH 5 | Zeta pH 7 |
| KC3-OA | 109.7 | 89.80 ± 3.53 | 21.64 | −3.65 |
| KC3-X1-H | 126.0 | 87.89 ± 4.8 | 28.33 | −2.81 |
| KC3-X1-K | 120.4 | 89.32 ± 45.4 | 26.02 | −1.62 |
| KC2-X1-PH5 | 93.2 | 79.51 ± 5.8 | 22.65 | −12.48 |
| KC3-X1-PH5 | 106.4 | 88.99 ± 3.5 | 25.98 | −6.95 |
| KC3-X1-PH4 | 136.5 | 97.51 ± 6.9 | 25.01 | −2.12 |
| KC3-X1-PH1 | 109.0 | 95.62 ± 7.9 | 23.71 | −4.69 |

To examine how effective these ICLs are at encapsulating mRNA and transfecting cells in vivo, CB6F1 hybrid mice were immunized i.m. in the caudal thigh with 1 or 0.2 ug mRNA and given a homologous boost on SD35 (FIG. 16A). Splenic T cell responses were analyzed 8 days post-boost by stimulating cells with pools of 15mer peptides overlapping by 11 residues in the presence of the golgi inhibitor brefeldin A. Cells were stained with fluorescently-tagged antibodies and analyzed by flow cytometry for intracellular IFN-g, TNF-α and IL-2.

The magnitudes of the CD4 T cell (FIG. 16B) and CD8 T cell (FIG. 16C) TB-specific responses were comparable between LNP formulations at both the 1 and 0.2 ug dose. Only the CD4 T cell response elicited by the KC3-X1-PH5 formulation was significantly greater than KC3-OA. Thus, LNPs comprised of these biodegradable ICLs were capable of eliciting robust antigen-specific T cell immunity at the doses tested.

SEQ ID NO: 417 is a polypeptide sequence consisting of the human LAMP-1 N-terminal signal peptide, a collected of covalently linked M. tuberculosis proteins, and the C-terminal human LAMP-1 terminal domain.

SEQ ID NO: 418 is a codon optimized RNA sequence corresponding to the polypeptide in SEQ ID NO. 417. The ORF is flanked by HBB UTRs.

### Example 10C. Comparing the In Vivo Immunogenicity of a Tuberculosis mRNA Vaccines Using KC3-X1-PH12 LNPs at Various ICL Mol % and after Lyophilization and Reconstitution

The primary purpose of this study was to determine how varying the mol % of KC3-X1-PH12 LNPs altered the activation and expansion of M. tuberculosis-specific T cells. A secondary purpose was to compare the immunogenicity of test articles stored at −20° C. versus the same test articles that were freeze-dried and reconstituted with water right before injection. A 2^{nd} generation Mtb mRNA targeted to the lysosome using the human LAMP-1 terminal domain (TD) was encapsulated in LNPs prepared with KC3-X1-PH12/DSPC/DPPS/Chol/PEG-DMG (48/5/5/40.5/1.5 mol %) or variants on this where the mol % of KC3-X1-PH12 increased to 52 or 56 mol % while the cholesterol content was reduced proportionately. The N/P ratio was 5.25 for all LNPs. In several experimental groups, a 2^{nd} gen LAMP-1 mRNA (SEQ ID NO: 408 encoding for SEQ ID NO: 407 below) was co-encapsulated at with three additional mRNAs encoding Mtb vaccine antigens of interest. These encoded Mtb vaccine clinical candidates M72 (SEQ ID NO: 439), Ag85A (SEQ ID NO: 446) and a PstS1-LpqH chimeric antigen (SEQ ID NO: 451). The M72 and Ag85A coding sequences were genetically flanked on the N- and C-terminus with a human CD169 SP signal peptide (SP; SEQ ID NO: 433 and terminal domain (TD; SEQ ID NO: 434), respectively. These synthetic antigens will be referred to as CD169-M72 and CD169-Ag85A. The PstS1-LpqH coding sequence was flanked on the N- and C-terminus with an influenza A H1 SP (SEQ ID NO: 32) and TD (SEQ ID NO: 33). This synthetic antigen will be referred to as H1-PstS1-LpqH. The polypeptide and corresponding RNA sequences for CD169-M72 are SEQ ID NOs: 438 and 439, respectively. The polypeptide and corresponding RNA sequences for CD169-Ag85A are SEQ ID NOs: 447 and 448. The polypeptide and corresponding RNA sequences for H1-PstS1-LpqH are SEQ ID NOs: 451 and 452. Both the CD169 and H1 TDs direct the antigen to the cell surface for presentation to B cells. Targeting genetically-encoded vaccine antigens to the cell surface has been shown to improve B cell responses and downstream Ab titers by increasing B cell receptor cross-linking. These multivalent mRNA-LNPs were formulated into LNPs comprising of KC3-X1-PH12/DSPC/DPPS-NH4/Chol/PEG-DMG at 52/5/5/36.5/1.5 mol % and an N/P of 5.25. The study design is shown in FIG. 21A.

The LNPs were prepared and analyzed as in Example 3 and Example 4. A table of the formulation compositions and their physicochemical characteristics are shown below in Table 10-C-1. Additionally, a portion of mRNA-LNPs made with 52 mol % KC3-X1-PH12 and 56 mol % KC3-X1-PH12 were lyophilized and reconstituted as described in Example 43.

Particle size refers to the Z-Average particle size after freezing and at an N/P ratio. Zeta refers to zeta potential measurements at either pH 5 or pH 7 in units of mV. mRNA encapsulation efficiency after freezing & thawing is referred to as (EE,%).

TABLE 10-C-1
Physicochemical properties of mRNA-LNPs used in Example 10C. In the first
column, Group # refers to the study Group # shown in FIG. 21A. For formulations in rows 1-3, a
single mRNA encoding a 2^{nd} generation TB synthetic antigen (SEQ ID NO: 406) was
encapsulated in KC3-X1-PH12/DPPS LNPs using a range of PH12 mol %. In rows 4-6, the 2^{nd}
generation TB mRNA was co-encapsulated with equal weights of three additional mRNAs
described in this example. Relative to the 2^{nd} generation mRNA, the quantity of these additional
mRNAs decreased with Group 13 containing the highest amount and Group 15 containing the
lowest amount.

| | | mol | | Z- | | % EE ± | Zeta pH | |
|---|---|---|---|---|---|---|---|---|
| Grp # | N/P | % | mRNA | Average | PDI | stdev | 5 | Zeta pH 7 |
| 1-3, 11, | 5.25 | 56 | 2^{nd} gen | 127.9 | 0.1085 | 92.50 ± | 25.63 | −0.2571 |
| 12 | | | LAMP-1 | | | 0.29 | | |
| 4-6, 9, | 5.25 | 52 | 2^{nd} gen | 127.8 | 0.0728 | 92.50 ± | 25.8 | 0.09415 |
| 10 | | | LAMP-1 | | | 0.22 | | |
| 7, 8 | 5.25 | 48 | 2^{nd} gen | 130.3 | 0.1126 | 92.95 ± | 24.41 | −0.0527 |
| | | | LAMP-1 | | | 0.23 | | |
| 13 | 5.25 | 52 | Quad | 94.62 | 0.0903 | 93.95 ± | 21.75 | 0.3309 |
| | | | | | | 0.18 | | |
| 14 | 5.25 | 52 | Quad | 135.0 | 0.109 | 90.79 ± | 23.65 | 0.03487 |
| | | | | | | 0.19 | | |
| 15 | 5.25 | 52 | Quad | 138.4 | 0.1117 | 90.21 ± | 24.44 | −0.7664 |
| | | | | | | 0.44 | | |

The formulations ranged in size from approximately 94 to 138 nm in diameter The % entrapped mRNA ranged from 90-94% and LNPs exhibited positive surface charge at pH 5~+22 to +26 mV and a negative charge at pH 7 −1 to +1 mV.

TABLE 10-C-2
Particle size (Z-ave), polydispersity (PDI) and mRNA
encapsulation efficiency (% EE) of LNPs that were
lyophilized and reconstituted (Post Lyo/R), made
with either 52 or 56 mol % KC3-X1-PH12 and VRN905.

| | | | Post Lyo/R | | Post Lyo/R |
|---|---|---|---|---|---|
| ICL | N/P | mol % | Z-ave | PDI | % EE |
| KC3-X1-PH12 | 5.25 | 52 | 157.3 | 0.07 | 78.55 ± 0.53 |
| KC3-X1-PH12 | 5.25 | 56 | 164.4 | 0.07 | 75.85 ± 1.04 |

After lyophilization, it was found that the particle size increased by about 30-35 nm for each, and the % EE was reduced by about 15% points.

CB6F1 female mice were vaccinated i.m. according to the study design shown in FIG. 21A. All test articles were stored at −20° C. before being thawed on ice and immediately injected into mice. mRNA-LNPs for Groups 9-12 were freeze-dried in a Hepes/Tris buffer as described in Example 43 and stored at −20° C. These were reconstituted with water prior to injection into mice. Mice were given a second injection on SD28 and spleens were collected on SD36 and T cell responses were quantified by peptide stimulation as described in Example 10.

At the 1 mcg dose for the monovalent formulations (Groups 1-12), the magnitude of the antigen-specific CD4 T cell response increased as the mol % of PH12 increased; PH12 at 56 mol % was statistically superior to 48 mol %. PH12 at 52 and 56 mol % were statistically similar, indicating that either mol % would elicit robust CD4 T cell responses. As expected, the magnitude of TB-specific CD4 cells decreased as the dose decreased due to limiting amounts of available antigen (compare FIGS. 21B-21D). For the CD8 T cell response, the size also increased with the mol % (FIG. 21E) but this was not statistically significant. The response magnitude at the 0.3 mcg dose was similar to that of the 1 mcg dose, and then it decreased at the 0.1 mcg dose. Importantly, the immunogenicity of the lyophilized and reconstituted formulations (Groups 9-12) was comparable to the non-lyophilized formulations. In conclusion, these data indicate that KC3-X1-PH12 at either 52 or 56 mol % is more optimal than 48 mol %. Stability is a critical consideration for vaccines, and we demonstrated here that lyophilized test articles did not decrease in immunogenicity despite increasing in size (Z-ave) and decreasing in encapsulation efficiency.

In mice vaccinated with quadrivalent mRNAs, the primary question we tested here was whether vaccination with additional mRNAs would adversely affect the immunogenicity of the 2^{nd} gen mRNA. Shown in FIGS. 21H and 21I, it appears that the co-delivery of the 3 additional mRNAs potentially decreased the size of the CD4 T cell response to the 2^{nd} generation antigen; however it did not have an adverse effect on the CD8 response. In conclusion, the KC3-X1-PH12 LNP formulation is able to co-deliver multivalent vaccine mRNAs.

SEQ ID NO. 407. Amino acid sequence of a synthetic antigen for an Mtb vaccine. The antigenic polypeptide is flanked on the N-terminus by the LAMP-1 signal peptide (SEQ ID NO. 35) and TM/CT on the C-terminus (SEQ ID NO. 36). The N- and C-terminal domains are underlined. SEQ ID NO. 408. Codon-optimized mRNA forward open reading frame sequence corresponding to the polypeptide in SEQ ID NO. 407. The sequence contains 3′ and 5′ HBB UTRs (SEQ ID NOs. 501 and 502).

In some embodiments, the mRNA can comprise one or more sequences selected from the list consisting of: SEQ ID NOs: 402, 404, 406, 408, 410, 412, 414, 416, 418, 420, 422, 424, 426, 428, 430, 432, 439, 441, 443, 446, 448, and 452.

In some embodiments, the mRNA can encode one or more proteins or peptides encoded by polypeptide sequences selected from the list consisting of: SEQ ID NOs: 401, 403, 405, 407, 409, 411, 413, 415, 417, 419, 421, 423, 425, 427, 429, 431, 438, 440, 442, 445, 447, and 451.

### Example 11. Preparation and Analysis of LNPs Containing Biodegradable ICLs Encapsulating Influenza a H5 mRNA Used to Evaluate T-Cell and B-Cell Responses in Mice

### Example 11A Comparing Immunogenicity of mRNA-LNPs Comprising KC3-OA, KC3-OA, KC3-X1-K, KC3-X1-S, KC4-X1-H, KC3-X1-PH5, KC3-X1-PH6, and KC3-X1-PH8 ICLs

The purpose of this study was to compare the immunogenicity of mRNA-LNPs consisting of KC3-OA or panel of novel biodegradable ICLs. To track B-cell and T-cell responses, we used an mRNA encoding an influenza A H5 antigen (SEQ ID NOs: 5 and 6). The ICLs KC3-OA, KC3-X1-K, KC3-X1-S, KC4-X1-H, KC3-X1-PH5, KC3-X1-PH6, and KC3-X1-PH8 were separately formulated in LNPs comprising of ICL/DSPC/DPPS-NH₄/Chol/PEG-DMG (52/5/5/36.5/1.5 mol %) at an N/P ratio of 5.25 and compared to an SM-102 control formulation (50/10/0/38.5/1.5 mol %) at an N/P ration of 6. An mRNA encoding the influenza A/Texas/37/2024 H5 antigen was encapsulated in all LNPs. Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 74 to 130 nm in diameter. The % entrapped mRNA ranged from 86-96% and LNPs exhibited positive surface charge at pH 5 (~+19 to +25 mV) and a negative to and close to neutral charge at pH 7 (−4 to +1.5 mV).

TABLE 11A-1
Physicochemical properties and characterization
of LNPs containing H5N1 A mRNA.

| Lipid | Particle | E.E, %, | Zeta pH | Zeta pH |
|---|---|---|---|---|
| Composition | Size (nm) | Stdev | 5 | 7 |
| KC3-OA | 104.90 | 93.15 ± 13.3 | 22.76 | 0.4832 |
| SM-102 | 74.32 | 85.84 ± 5.7 | 19.2 | −4.144 |
| KC3-X1-K | 103.70 | 96.10 ± 2.2 | 21.67 | 0.607 |
| KC3-X1-S | 109.20 | 85.99 ± 3.9 | 22.59 | 0.4785 |
| KC4-X1-H | 129.80 | 96.22 ± 2.5 | 22.73 | 1.449 |
| KC3-X1-PH5 | 104.60 | 96.45 ± 4.8 | 24.7 | −3.022 |
| KC3-X1-PH6 | 103.30 | 91.89 ± 8.5 | 21.26 | −1.703 |
| KC3-X1-PH8 | 94.46 | 91.62 ± 3.7 | 19.14 | −2.727 |

To examine how effective these ICLs are at encapsulating mRNA and transfecting cells in vivo, BALB/c mice were immunized i.m. in the caudal thigh with 0.1 ug mRNA and given a homologous boost on SD22 (FIG. 11A). Serum was collected on SD21 and SD35 to quantify antibody levels. Splenic T cell responses were analyzed on SD49 using the methods in Example 38. Cells were stained with fluorescently-tagged antibodies and analyzed by flow cytometry for intracellular IFN-g, TNF-α and IL-2. Total HA-binding IgG titers were quantified by standard limiting-dilution ELISA. HA-binding IgG titers on SD21 (FIG. 11B) and SD34 (13 days post-boost; FIG. 11C) were similar across groups. H5-specific CD4 (FIG. 11D) and CD8 T cell (FIG. 11E) memory pools were similar in magnitudes across ICLs. KC4-X1-H trended lower in all readouts, but this was not statistically significant relative to other groups. In conclusion, the ionizable lipids KC3-X1-K, KC3-X1-S, KC4-X1-H, KC3-X1-PH5, KC3-X1-PH6 and KC3-X1-PH8 readily formed into LNPs and had similar activity in vivo compared to the KC3-OA and commercial SM-102 formulations.

SEQ ID NO: 5 is a polypeptide sequence consisting of the influenza A H5 A/Texas/37/2024 glycoprotein.

SEQ ID NO: 6 is a codon optimized RNA sequence corresponding to the polypeptide in SEQ ID NO: 5. The ORF is flanked by HBB UTRs.

### Example 11B Comparing Immunogenicity of mRNA-LNPs Comprising KC3-OA, KC3-X1-PH5, KC3-X1-K, KC3-X1-S, KC4-X1-H ICLs

The purpose of this study was to compare the immunogenicity of mRNA-LNPs consisting of KC3-OA or a panel of novel biodegradable ICLs. To track B-cell and T-cell responses, we used an mRNA encoding an influenza A H5 antigen (SEQ ID NOs: 15 and 16). The ICLs KC3-X1-PH5, KC3-X1-K, KC3-X1-S, KC4-X1-H were separately formulated in LNPs comprising of ICL/DSPC/DPPS-NH4/Chol/PEG-DMG (52/5/5/36.5/1.5 mol %) at an N/P ratio of 5.25 and compared to a KC3-OA formulation (48/5/5/40.5/1.5 mol %) with an N/P ratio of 5.25. All LNPS encapsulated an mRNA encoding an influenza A/Texas/37/2024 H5 glycoprotein. Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 110 to 141 nm in diameter. The % entrapped mRNA ranged from 90-95% and LNPs exhibited positive surface charge at pH 5 (~+17 to +24 mV) and a negative charge at pH 7 (−4 to −8 mV).

TABLE 11B-1
Physicochemical properties and characterization
of LNPs containing an influenza A H5 mRNA.

| | Particle | E.E, %, | Zeta pH | Zeta pH |
|---|---|---|---|---|
| Lipid Composition | Size (nm) | Stdev | 5 | 7 |
| KC3-OA | 113.70 | 93.51 ± 8.4 | 17.04 | −3.87 |
| KC3-X1-PH5 | 110.50 | 95.44 ± 4.5 | 22.16 | −7.77 |
| KC3-X1-K | 123.10 | 92.51 ± 4.7 | 22.86 | −3.97 |
| KC3-X1-S | 117.10 | 89.80 ± 2.0 | 22.70 | −6.64 |
| KC4-X1-H | 141.10 | 93.19 ± 1.7 | 21.13 | −4.01 |

To examine how effective these ICLs are at encapsulating mRNA and transfecting cells in vivo, BALB/c mice were i.m. in the caudal thigh with 0.1 or 0.02 ug mRNA and given a homologous boost on SD22 (FIG. 12A). Serum was collected on SD35 (13-days post-boost) to quantify antibody levels. Splenic T cell responses were analyzed on SD49 using the methods outlined in Example 38. Total HA-binding IgG titers were quantified by standard limiting-dilution ELISA.

The KC3-X1-PH5 and KC3-OA formulations elicited equivalent antibody titers at both dose levels. For the 0.1 ug dose (FIG. 12B), titers for the KC3-X1-K, KC3-X1-S and KC3-X1-H groups were lower than KC3-X1-PH2. For the 0.02 ug dose (FIG. 12C), all groups had comparable titers. The size of the HA-specific CD4 T cell (FIG. 12D) and CD8 T cell responses (FIG. 12E) were comparable at both dose levels with the exception of the CD4 response for KC3-X1-H group at the 0.1 dose—it was significantly greater than that of the KC3-OA formulation. In conclusion, the novel lipids KC3-X1-PH5, KC3-X1-K, KC3-X1-S, KC4-X1-H had comparable activity to KC3-OA in eliciting antibody and T cell responses to a vaccine antigen.

SEQ ID NO: 15 is a polypeptide sequence consisting of an influenza A H5 A/Texas/37/2024 glycoprotein. SEQ ID NO: 16 is a codon optimized RNA sequence corresponding to the polypeptide in SEQ ID NO. 15. The ORF is flanked by HBB UTRs.

### Example 11C Comparing Immunogenicity of mRNA-LNPs Comprising KC3-OA, KC3-X1-H, KC3-X1-PH1, KC3-X1-K, KC3-X1-PH5, KC3-X1-M, KC3-X1-L, KC2-X1-PH5, and KC3-X1-PH4 ICLs

The purpose of this study was to compare the immunogenicity of mRNA-LNPs consisting of KC3-OA or a panel of novel biodegradable ICLs. To track B-cell and T-cell responses, we used an mRNA encoding an influenza A H5 antigen (SEQ ID NOs: 5 and 6). The ICLs KC3-OA, KC3-X1-H, KC3-X1-PH1, KC3-X1-K, KC3-X1-PH5, KC3-X1-M, KC3-X1-L, KC2-X1-PH5, KC3-X1-PH4 were separately formulated in LNPs comprising of ICL/DSPC/DPPS-NH4/Chol/PEG-DMG (52/5/5/36.5/1.5 mol %) at an N/P ratio of 5.25 and encapsulating an mRNA encoding the influenza A/Texas/37/2024 H5 glycoprotein. Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 88 to 128 nm in diameter. The % entrapped mRNA ranged from 72-97% and LNPs exhibited positive surface charge at pH 5 (~+20 to +29 mV) and a close to neutral or negative charge at pH 7 (+3 to −10 mV).

TABLE 11C-1
Physicochemical properties and characterization
of LNPs containing influenza A H5 mRNA.

| Lipid | Particle | E.E, %, | | |
|---|---|---|---|---|
| Composition | Size (nm) | Stdev | Zeta pH 5 | Zeta pH 7 |
| KC3-OA | 99.55 | 86.7 ± 5.0 | 21.63 | 2.90 |
| KC3-X1-H | 112.1 | 80.2 ± 1.8 | 26.91 | −0.13 |
| KC3-X1-PH1 | 121.6 | 92.5 ± 2.2 | 29.42 | 0.57 |
| KC3-X1-K | 126.3 | 77.2 ± 2.2 | 25.73 | 2.01 |
| KC3-X1-PH5 | 100.4 | 87.6 ± 3.0 | 26.10 | −3.79 |
| KC3-X1-M | 113.5 | 75.9 ± 1.7 | 25.73 | −1.20 |
| KC3-X1-L | 116.5 | 72.9 ± 1.9 | 20.14 | −0.90 |
| KC2-X1-PH5 | 87.68 | 80.2 ± 2.2 | 23.51 | −9.94 |
| KC3-X1-PH4 | 128.2 | 97.0 ± 6.6 | 25.10 | −0.29 |

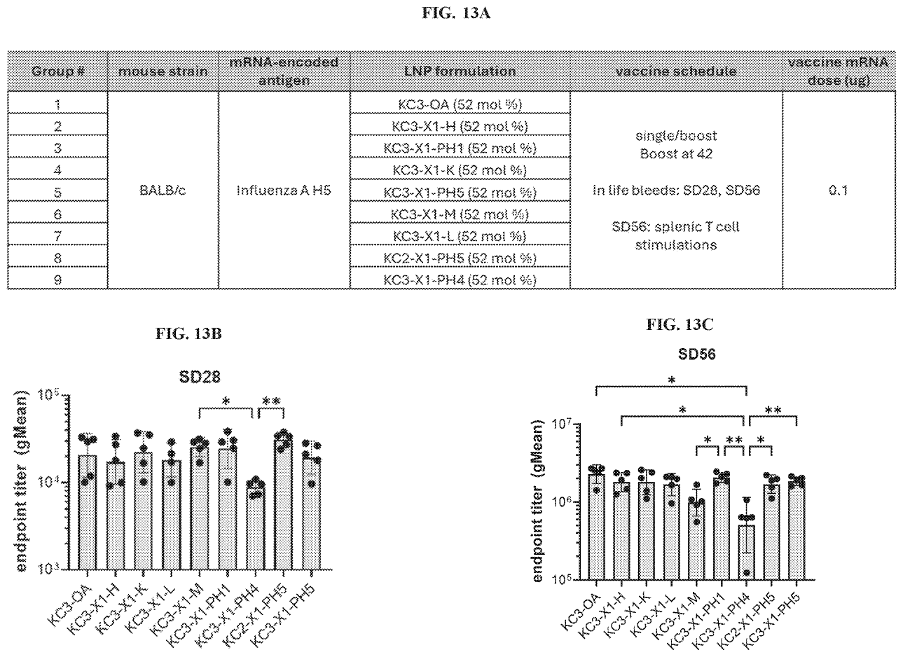

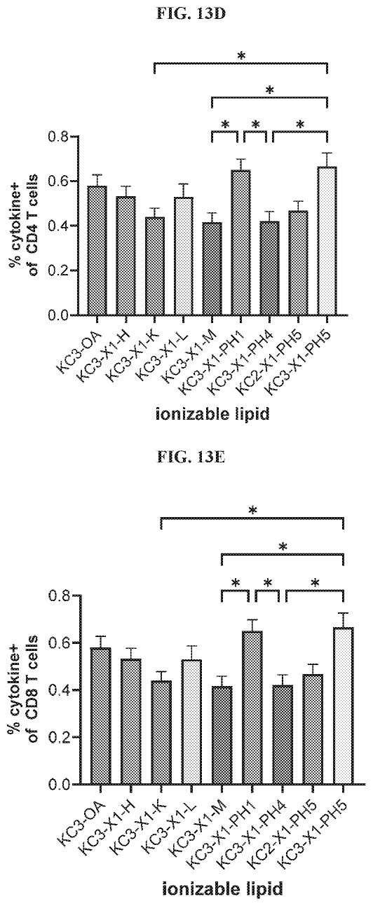

To compare the ability of mRNA-LNPs formulated with these ICLs to induce B- and T-cell responses, BALB/c mice were i.m. in the caudal thigh with 0.1 ug mRNA and given a homologous boost on SD42 (FIG. 13A). Serum was collected on SD28 and SD56 (14 days post-boost) to quantify antibody levels. Splenic T cell responses were analyzed on SD56 using the methods outlined in Example 38. Total HA-binding IgG titers were quantified by standard limiting-dilution ELISA.

On SD28 and SD56, the KC3-X1-PH4 ICL formulation induced statistically inferior antibody titers compared to the other ICLs (FIGS. 13B and 13C). KC3-X1-M also had lower titers on SD56. All other ICLs induced comparable levels of anti-H5 IgG antibodies. All formulations generated H5-specific CD4 (FIG. 13D) and CD8 (FIG. 13E) T cell responses, with the KC3-X1-PH1 and KC3-X1-PH5 ICLs outperforming the KC3-X1-K, KC3-X1-M and KC3-X1-PH4 formulations. In conclusion, all of the novel ICLs tested in this study elicited antibodies and T cell responses. Depending on the readout, KC3-X1-M and KC3-X1-PH4 had worse activity compared to the other ICLs.

We also sought to compare the acute inflammatory signature of these ICLs to that of KC3-OA. Acute induction of pro-inflammatory factors is critical for mobilizing the innate immune system. However, we reasoned that if any of these ICLs exhibited a clearly elevated inflammatory signature relative to the other formulations, this ICL may cause increased rates of adverse events in people. Different BALB/c mice were injected with 1 ug i.m. of the same formulations shown in Table 11C-1. Blood and draining lymph nodes (dLNs; ipsilateral, inguinal, and iliac LNs) were collected 6 hrs post injection. Serum was isolated in serum separator tubes and LNs were lysed in 0.25 mL T-PER buffer with DNase I. Samples were frozen and stored at −80° C. until cytokines and chemokines were quantified using the LEGENDplex 13-plex Mouse Anti-Virus Response Panel (BioLegend, Cat. NO. 740622). Baseline analyte concentrations were measured in mice that did not receive an injection.

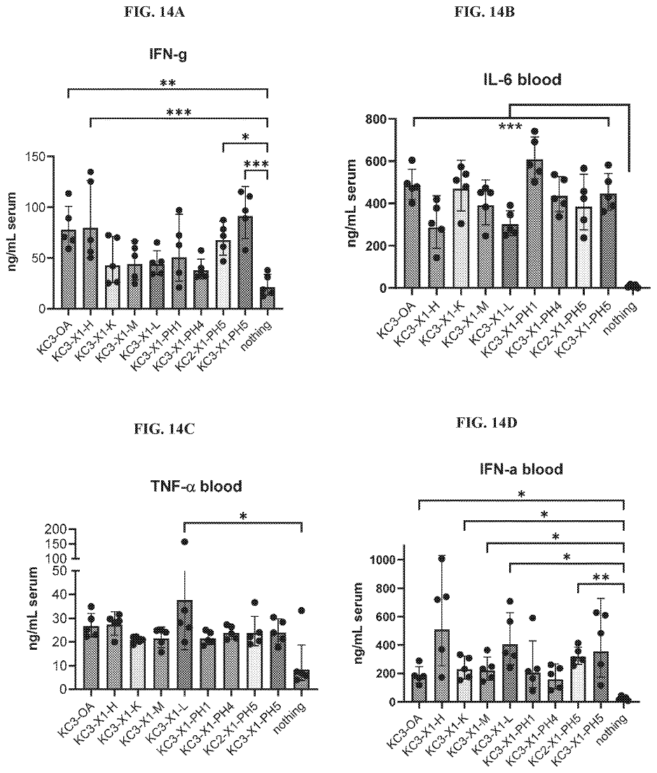

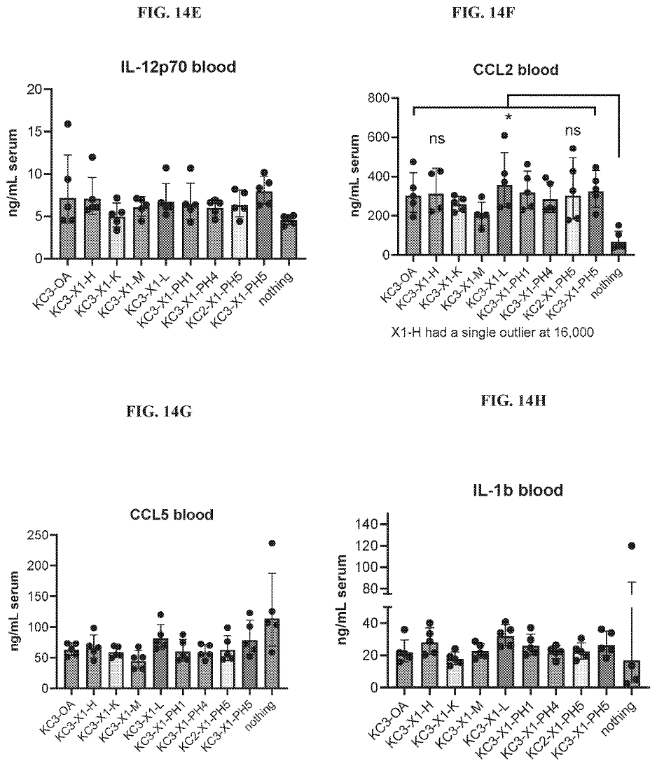

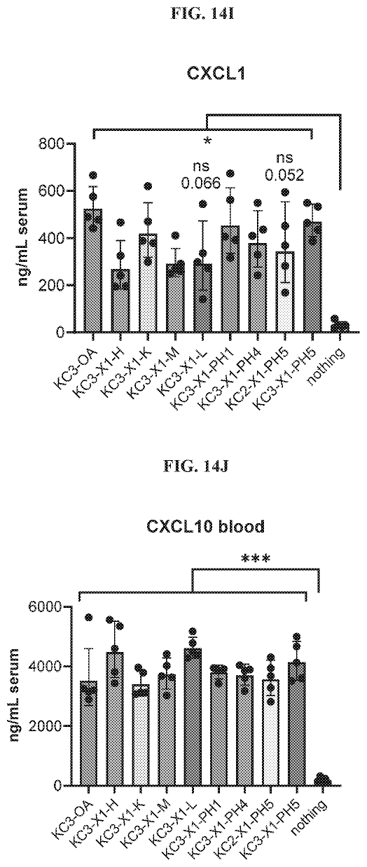

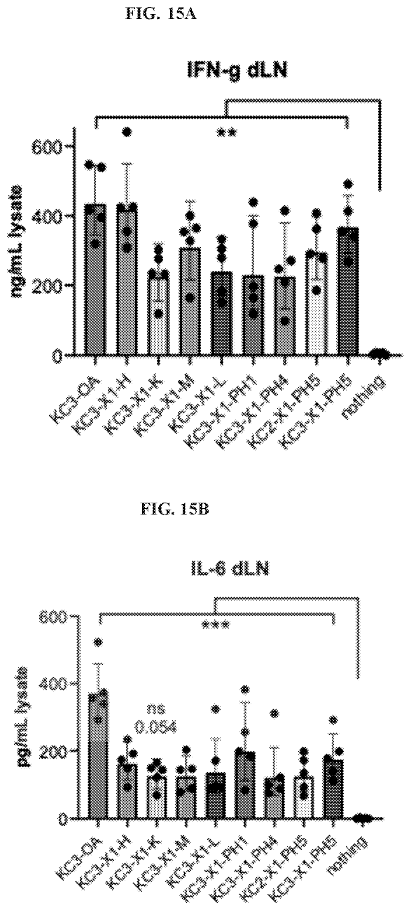

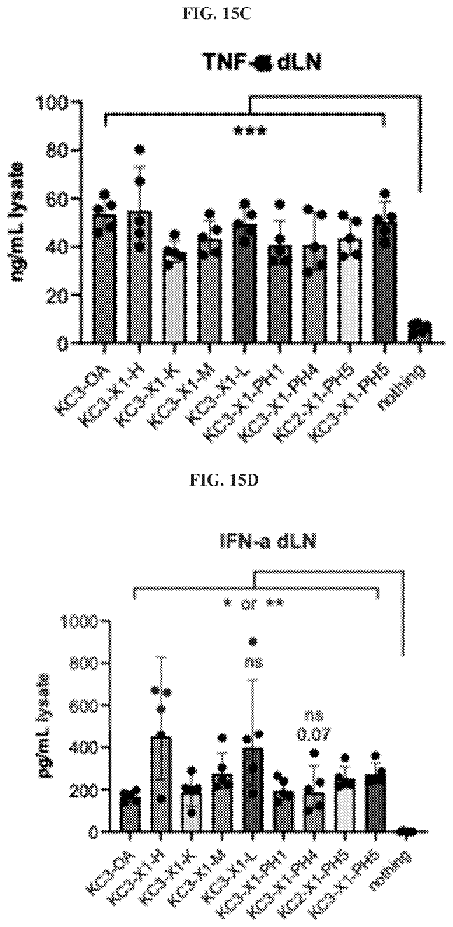

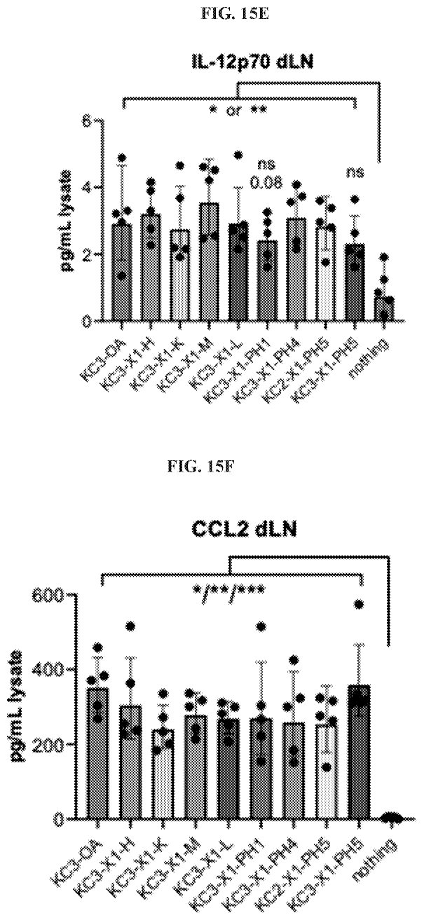

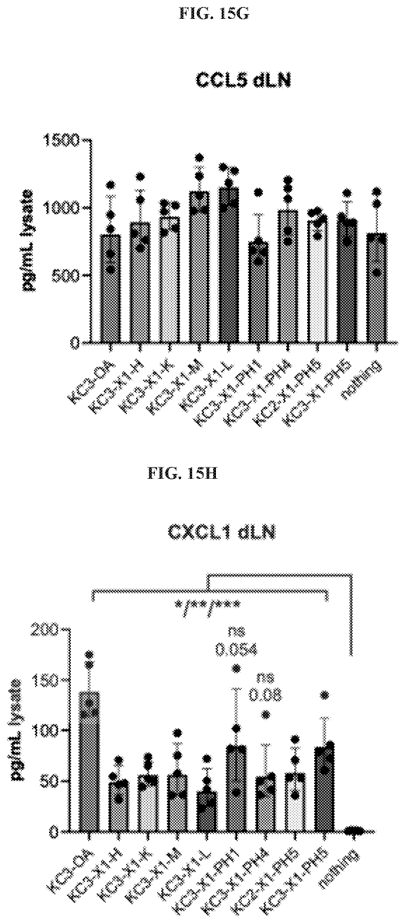

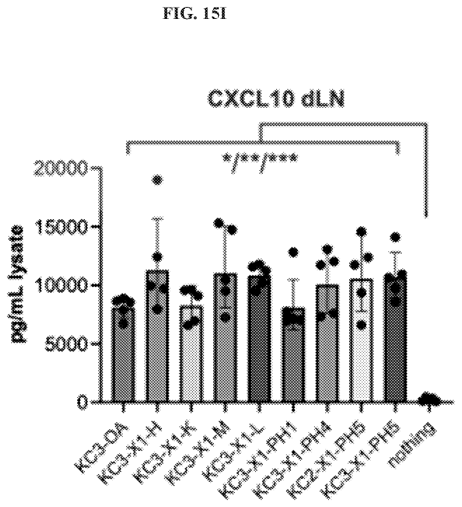

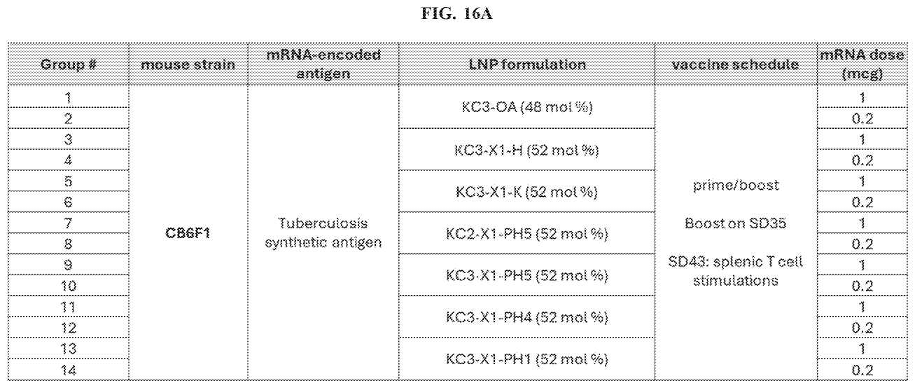

All LNP formulations induced qualitatively similar acute pro-inflammatory signatures in the blood (FIGS. 14A-14J) and draining LNs (FIGS. 15A-15I, indicating that they signal through the same pattern recognition receptors. None of the ICLs were outliers across multiple analytes in inducing excessive production. In the blood, only KC3-OA, KC3-X1-H, KC2-X1-PH5 and KC3-X1-PH5 induced IFN-g production (FIG. 14A) above baseline. All or almost all LNP formulations induced IL-6 (FIG. 14B), type I interferon-alpha (FIG. 14D), CCL2 (FIG. 14F), CXCL1 (FIG. 141) and CXCL10 (FIG. 14J). In the local draining LNs, IFN-g (FIG. 15A), IL-6 (FIG. 15B), TNF-α (FIG. 15C), IFN-alpha (FIG. 15D), IL-12p70 (FIG. 15E), CCL2 (FIG. 15F), CXCL1 (FIG. 15H) and CXCL10 (FIG. 151) were almost uniformly and comparably expressed across all LNP formulations. In conclusion, all mRNA-LNPs formulated with KC3-OA, KC3-X1-H, KC3-X1-PH1, KC3-X1-K, KC3-X1-PH5, KC3-X1-M, KC3-X1-L, KC2-X1-PH5, and KC3-X1-PH4 ICLs comparably trigger pattern recognition receptor signaling and downstream production of cytokines and chemokines.

### Example 1 1D Comparing Immunogenicity of mRNA-LNPs Comprising KC3-X1-PH5, KC3-OA, KC3-X1-AA, KC2-X2-K9 and KC3-X1-PH12 ICLs

The purpose of this study was to compare the immunogenicity of mRNA-LNPs consisting of KC3-OA or a panel of novel biodegradable ICLs. We also examined whether the mol % of the ICL KC3-X1-PH5 affected immunogenicity.

To track B-cell and T-cell responses, we used an mRNA encoding the influenza A/Texas/37/2024 H5 antigen (SEQ ID NOs: 5 and 6). The ICLs KC3-X1-PH5, KC3-OA, KC3-X1-AA, KC2-X2-K9 and KC3-X1-PH12 separately formulated in LNPs comprising of ICL/DSPC/DPPS-NH4/Chol/PEG-DMG (52/5/5/36.5/1.5 mol %) at an N/P ratio of 5.25. KC3-X1-PH5 was also formulated in LNs comprising of 48 mol % (48/5/5/40.5/1.5 mol %) and 56 mol % (56/5/5/32.5/1.5 mol %). Particle size refers to the Z-Average particle size after freezing and thawing. Zeta refers to zeta potential measurements at either pH 5 or pH 7 in units of mV. mRNA encapsulation efficiency is referred to as (E.E,%). The formulations ranged in size from approximately 102 to 124 nm in diameter. The % entrapped mRNA ranged from 85-93% and LNPs exhibited positive surface charge at pH 5 (~+110 to +17 mV) and a close to neutral or negative charge at pH 7 (−3 to −5 mV).

TABLE 11D-1
Physicochemical properties and characterization of
LNPs containing an influenza A H5 vaccine mRNA.

| | Particle Size | E.E, %, | | |
|---|---|---|---|---|
| Lipid Composition | (nm) | Stdev | Zeta pH 5 | Zeta pH 7 |
| KC3-X1-PH5 | 101.50 | 92.9 ± 3.8 | 16.7 | −3.0 |
| 48 mol % | | | | |
| KC3-X1-PH5 | 107.70 | 93.1 ± 3.8 | 13.0 | −3.1 |
| 52 mol % | | | | |
| KC3-X1-PH5 | 119.90 | 91.2 ± 3.1 | 12.6 | −4.2 |
| 56 mol % | | | | |
| KC3-OA | 106.00 | 89.6 ± 2.2 | 11.5 | −4.5 |
| 48 mol % | | | | |
| KC3-X1-AA | 104.30 | 89.5 ± 2.2 | 11.3 | −4.4 |
| 52 mol % | | | | |
| KC3-X2-K9 | 124.00 | 91.9 ± 3.2 | 14.0 | −2.8 |
| 52 mol % | | | | |
| KC3-X1-PH12 | 102.60 | 85.4 ± 4.8 | 13.1 | −3.6 |
| 52 mol % | | | | |

To compare the ability of mRNA-LNPs formulated with these ICLs to induce B- and T-cell responses, BALB/c mice were i.m. in the caudal thigh with 0.1 ug or 0.02 ug mRNA and given a homologous boost on SD22 (FIG. 17A). Serum was collected on SD21 and SD35 (14-days post-boost) to quantify antibody levels. Splenic T cell responses were analyzed on SD35 using the methods outlined in Example 38. Total HA-binding IgG titers were quantified by standard limiting-dilution ELISA.

Total H5 binding IgG titers 3 weeks post primary vaccination were similar between the formulations at both doses with only KC3-X1-PH12 (52 mol %, 0.1 ug dose) being statistically lower than titers from the KC2-X1-PH5 52 mol % (FIGS. 17B and D). After the 2^{nd} immunization, all titers were boosted. At the 0.1 ug dose (FIG. 17C), the KC3-X1-PH5 formulation (48 mol %) induced significantly higher titers than those of KC3-OA, KC3-X1-AA, KC2-X2-K9 and KC3-X1-PH12. At the 0.02 ug dose (FIG. 17E), KC3-X1-PH5 (48 mol %) was only higher than the KC3-X1-AA group. In conclusion, formulations containing the novel biodegradable ICLs elicited H5 binding IgG titers equivalent to KC3-OA. The data indicate that KC3-X1-PH5 is optimally formulated into LNPs at 48 mol %, although this was not statistically higher than the 52 and 56 mol % LNPs. H5-specific T cell responses were also analyzed in the spleen. Interestingly, the magnitude of the CD4 T cell responses in the 0.1 ug groups positively correlated with the mol % of KC3-X1-PH5, suggesting that 48 mol % may be better suited for B cell responses and 56 mol % may be better for T cells (FIG. 17F). There was a positive trend in the CD8 response as the mol % of KC3-X1-PH5 increased, but this was not statistically significant (FIG. 17H). In the 0.02 ug dose groups, the CD4 (FIG. 17G) and CD8 (FIG. 171) responses were smaller than at the 0.1 ug dose level, but there was no difference in the magnitude between the formulations. In conclusion, all tested biodegradable ICLs could be formulated in mRNA-LNPs that were immunogenic in vivo. The mol % of KC3-X1-PH5 appeared to be influence the antibody and T cell responses: a lower mol % may be better for eliciting vaccine antibodies, whereas a higher mol % may be better for T cell responses.

### Example 11E Comparing Immunogenicity of mRNA-LNPs Comprising SM-102, KC3-X1-PH5 and KC3-X1-PH12 ICLs at Various Mol % and N/P Ratios

mRNA encoding influenza H5 polypeptide antigens were formulated separately into LNPs comprising an ICL/DSPC/DPPS-NH4/Chol/PEG-DMG where the ICL mol % was 48, 52 or 56 mol %, DSPC and DPPS-NH4 present at 5 mol % each, PEG-DMG at 1.5 mol % and the remainder lipid was cholesterol. LNPs were formulated at an N/P ratio of 4.5 or 5.25. The SM-102 LNP formulation and the KC3-based LNPs were formulated using a ChipShop 187 microfluidic mixer chip at a flow rate of 12 mL/min and analyzed as in Example 4. The composition of each formulation and a panel of physical characteristics are shown below in Table 11E-1. The mRNA sequences encoding the native influenza A H5 and a stabilized H5 variant (Dosey et al. 2025. bioRXiv. https://doi.org/10.1101/2025.07.30.667762) are shown in SEQ ID NO: 6 and SEQ ID NO: 16, respectively.

Particle size refers to the Z-Average particle size after freezing and thawing. Zeta refers to zeta potential measurements at either pH 5 or pH 7 in units of mV. mRNA encapsulation efficiency after freezing & thawing is referred to as (E.E,%).

TABLE 11E-1

| ICL (mol %, N/P | | Z- | | | Zeta pH | Zeta pH |
|---|---|---|---|---|---|---|
| ratio) | mRNA | Average | PDI | % EE | 5 | 7 |
| SM-102 (50%, 6.0) | SEQ ID NO: 6 | 96.2 | 0.02 | 90.67 ± 0.73 | 12.7 | −3.64 |
| KC3-X1-PH12 | SEQ ID NO: 16 | 112.5 | 0.11 | 85.98 ± 0.96 | 17.2 | −1.64 |
| (48%, 4.5) | | | | | | |
| KC3-X1-PH12 | SEQ ID NO: 16 | 115.9 | 0.11 | 85.05 ± 1.04 | 18.3 | −1.16 |
| (52%, 4.5) | | | | | | |
| KC3-X1-PH12 | SEQ ID NO: 16 | 116.2 | 0.07 | 87.64 ± 1.15 | 16.8 | −1.82 |
| (52%, 5.25) | | | | | | |
| KC3-X1-PH12 | SEQ ID NO: 16 | 124.0 | 0.00 | 84.18 ± 1.2 | 16.9 | −1.73 |
| (56%, 4.5) | | | | | | |
| KC3-X1-PH5 | SEQ ID NO: 16 | 89.8 | 0.05 | 95.83 ± 0.40 | 18.7 | −2.95 |
| (48%, 5.25) | | | | | | |
| KC3-X1-PH5 | SEQ ID NO: 16 | 92.2 | 0.08 | 95.37 ± 0.87 | 17.3 | −1.88 |
| (52%, 5.25) | | | | | | |
| KC3-X1-PH5 | SEQ ID NO: 16 | 98.5 | 0.05 | 94.90 ± 1.18 | 17.0 | −1.31 |
| (56%, 5.25) | | | | | | |

The formulations ranged in size from approximately 90 to 124 nm in diameter. The % E.E ranged from 81-96% and the LNPs exhibited positive surface charge at pH 5 ~+13-19 and a negative charge at pH 7 −4 to −1 mV. While the size and encapsulation efficiency of the SM-102 formulation were well within the range of the other formulations for these parameters, it had the lowest zeta potential at pH 5 and pH 7 of all the samples measured.

Using the methods outlined in Example 10, mice were dosed intramuscularly (i.m.) with either 0.1 or 0.02 μg (see FIG. 18 for study design). Serum was collected on study day (SD) 21 for measuring antibody (Ab) titers and mice were then given a homologous boost immunization. Serum was collected 2 weeks post boost (SD35) and spleens were collected on SD49 to measure H5-specific T cell responses.

While only statistically significant compared to some groups, the SM-102 formulation tended to have the lowest binding IgG titers, both at the 0.1 and 0.02 μg doses (FIGS. 19A-19D). At both doses PH12 was generally more immunogenic when used at 52 or 56 mol % and there was not a consistent difference in Ab titers when an N/P ratio of 4.5 or 5.25. For PH5, at the 0.1 μg dose this ICL was immunogenic at 48, 52 and 56 mol %. At the 0.02 μg dose, 48 and 52 mol % with an N/P of 5.25 seemed to be preferable. T cell responses in the spleen were only quantified in mice vaccinated with PH12 and PH5 LNPs using 52 or 56 mol %, and these were compared to the SM-102 formulation. Overall all formulations elicited similar magnitude H5-specific CD4 and CD8 T cells (FIGS. 19E and 19F). These data indicate that B cell responses and circulating Ab levels may be more sensitive to small changes in immunogenicity caused by variations in the ICL mol % and N/P ratio. In contrast, at least at the doses tested, T cell expansion may be less sensitive.

### Example 11F Evaluation of the In Vivo Inflammatory Profile of LNP Compositions Comprising KC3-X1-PH5 and KC3-X1-PH12 ICLs

The purpose of this study was to explore whether LNPs comprised with the biodegradable ICLs KC3-X1-PH5 and KC3-X1-PH12 have a similar acute pro-inflammatory profile to that of the poorly biodegradable ICL KC3-OA. We have found that KC3-OA has a similar pro-inflammatory profile (focusing on the key cytokines IL-la, IL-6, TNF-α, IFN-γ and IL-12p70) to the ALC-0315 and SM-102 ICLs used in approved Covid-19 vaccines. While it is difficult to predict how vaccine reactogenicity will translate from pre-clinical animal models to humans, the comparator formulations used in the Covid-19 vaccines have been shown to have good safety profiles. We reasoned that by comparing the inflammatory profiles of LNPs comprised of these KC3-X1 ICLs to FDA approved benchmarks, we could get an early readout on potential problematic reactogenicity prior to entering the clinic.

LNPs entrapping mRNA encoding a stabilized influenza A H5 protein (SEQ ID NO: 16) were prepared and analyzed as described in Example 3 and Example 4. The formulations tested consisted of SM-102/DSPC/Chol/PEG-DMG (50/10/38.5.1.5 mol %), N/P=6; KC3-OA/DSPC/DPPS/Chol/PEG-DMG (48/5/5/40.5/1.5 mmol %, N/P=5.25; KC3-X1-PH12 (56/5/5/32.5/1.5 mol %), N/P=4.5; KC3-X1-PH5 (52/5/5/36.5/1.5 mol %), N/P=5.25).

Particle size refers to the Z-Average particle size after freezing and thawing. Zeta refers to zeta potential measurements at either pH 5 or pH 7 in units of mV. mRNA encapsulation efficiency after freezing & thawing is referred to as (E.E,%).

TABLE 11F-1
Physicochemical properties and characterization
of LNPs containing influenza A H5 mRNA.

| LNP Composition | Z-Average | | | zeta | Zeta |
|---|---|---|---|---|---|
| Lipids | Size (nm) | PDI | % E.E | pH 5 | pH 7 |
| SM-102 | 88.68 | 0.12 | 89.19 ± 0.67 | 11.5 | −1.28 |
| KC3-OA | 91.5 | 0.08 | 93.11 ± 0.13 | 15.3 | −0.38 |
| KC3-X1-PH12 (56% 4.5) | 124.00 | 0.00 | 84.18 ± 1.20 | 16.9 | −1.73 |
| KC3-X1-PH5 (52% 5.25) | 92.23 | 0.08 | 95.37 ± 0.87 | 17.3 | −1.88 |

The formulations ranged in size from approximately 89 to 124 nm in diameter The % entrapped mRNA ranged from 84-95% and LNPs exhibited positive surface charge at pH 5 +12-17 and a negative charge at pH 7-2-0 mV.

The study design is shown in FIG. 22A. BALB/c mice were injected i.m. in the caudal thigh with either 2.4 or 0.3 mcg mRNA. 6 hrs post injection, blood was collected via submandibular puncture into serum separation tubes; these were centrifuged at 10k×g for 10 min at 4° C. and serum was collected and stored at −80° C. until analysis. The thigh muscle-draining lymph node chain (dLNs; popliteal, iliac, inguinal) was collected and pooled into tubes containing cold PBS. LNs were transferred into tubes containing 250 uL TPER tissue protein extraction buffer (ThermoFisher, 78510) containing DNase I and HALT protease inhibitor cocktail (ThermoFisher, 78441). LNs were manually lysed using a pestle, left at room temperature for approximately 15 min to allow for DNAse activity, centrifuged at 10k×g for 10 min at 4° C., and clarified supernatant was stored at −80° C. until analysis. Analysis was done using the BioLegend LEGENDplex Mouse Anti-Virus Response Panel (BioLegend #740622) that measured IFN-7, CXCL1 (KC), TNF-α, CCL2 (MCP-1), IL-12p70, CCL5 (RANTES), IL-10, CXCL10 (IP-10), GM-CSF, IL-10, IFN-0, IFN-α, IL-6. Within each dose, statistical analysis was done using one-way ANOVA assuming lognormality (Brown-Forsythe test) and unequal geometric SDs (Welch ANOVA test).

Intramuscular injection of all four LNP formulations increased local and systemic concentrations of canonical pro-inflammatory cytokines and chemokines, and 2.4 mcg mRNA-LNPs induced higher levels than the 0.3 mcg dose. In the dLNs, there was upregulation of IFN-γ (FIG. 22B), TNF-α, (FIG. 22C), IL-6 (FIG. 22E), IL-1β (FIG. 22F), IFN-β (FIG. 22G), IFN-α, (FIG. 22H), and the chemokines CXCL1 (FIG. 22I) and CCL2 (FIG. 22J). Analytes concentrations between formulations were statistically similar with the exception of type I IFN-α and IFN—P: SM-102 induced significantly higher concentrations than all three of the KC3-based ICLs. Systemically (blood serum concentrations), the same analytes were upregulated (see FIGS. 22K-S). SM-102 induced statistically significantly higher concentrations of IFN-γ (FIG. 22K), TNF-α (FIG. 22L), IFN-β (FIG. 22P), IFN-α (FIG. 22Q), and CCL2 (FIG. 22S) than the KC3-based ICLs. There was no statistical difference between KC3-OA, KC3-X1-PH5 and KC3-X1-PH12. Altogether, these data show that SM-102 and the KC3-based ICLs signal through the same pathways, but that SM-102 is more inflammatory, both locally and systemically. High blood levels of these cytokines could lead to increased chances of reactogenicity such as fever and chills after vaccination with SM-102 compared to KC3-OA, KC3-X1-PH5 and KC3-X1-PH12.

It was determined that lyophilized and reconstituted samples retained ~90% activity of the frozen samples, while the LNP size increased ~30 nm after reconstitution and the % EE was reduced by approximately 6% compared to the frozen or refrigerated material. Multiple freeze-thaws of the frozen LNPs did not impact activity. Non-frozen LNPs after 7 days at 2-8° C. were as active as the frozen reference, if not better. There were no clear trends observable from mixing the LNPs at either concentration, or whether filtering the lipid mixture ethanolic solution made a difference.

### Example 12. Comparing the In Vivo Immunogenicity of Quadrivalent mRNA-LNPs Comprised of KC3-X1-PH12 LNPs or KC3-X2-PH12 LNPs at Various ICL Mol % and N/P Ratios

Four mRNAs encoding an influenza A H1 (SEQ ID NOs: 12 and 13), an influenza H3 (SEQ ID NOs: 14 and 914), a B/Victoria-like HA (SEQ ID NOs: 915 and 17) and a stabilized RSV preF (SEQ ID NOs: 9 and 10) were combined and formulated into LNPs comprising of KC3-X1-PH12/DSPC/DPPS-NH4/Chol/PEG-DMG where the ICL mol % was 52 or 56 mol %, DSPC and DPPS-NH4 were present at 5 mol % each, PEG-DMG at 1.5 mol % and the remainder lipid was cholesterol. KC3-X1-PH12 was also used in a similar formulation to encapsulate a single H5 mRNA (SEQ ID NO: 16) at an N/P ratio of 5.25 and either 52 or 56 mol %. The LNPs were prepared and analyzed as in Example 3 and Example 4. Table 12-A-1 shows the LNP compositions and their physicochemical characteristics. Additionally, a portion of LNPs made with 52 mol % KC3-X1-PH12 and 56 mol % KC3-X1-PH12 were lyophilized. Their characteristics after reconstitution are shown in Table 12-A-2.

Particle size refers to the Z-Average particle size after freezing and at an N/P ratio. Zeta refers to zeta potential measurements at either pH 5 or pH 7 in units of mV. mRNA encapsulation efficiency after freezing & thawing is referred to as (E.E,%).

TABLE 12A-1
Physicochemical properties of mRNA-LNP formulations used in Example 50.
Particle size (Z-ave), polydispersity (PDI) and mRNA encapsulation efficiency (% EE).

| ICL | N/P | mol % | Z-Ave | PDI | % EE ± stdev | Zeta pH 5 | Zeta pH 7 |
|---|---|---|---|---|---|---|---|
| KC3-X1-PH12 | 5.25 | 52 | 111.1 | 0.054 | 88.5 ± 0.25 | 16.76 | 0.47 |
| KC3-X1-PH12 | 5.25 | 56 | 116.1 | 0.047 | 88.4 ± 0.29 | 19.70 | 0.12 |
| KC3-X2-PH12 | 4.25 | 52 | 116.8 | 0.056 | 85.3 ± 1.01 | 9.13 | 0.30 |
| KC3-X2-PH12 | 5.25 | 56 | 106.1 | 0.048 | 93.2 ± 0.75 | 15.49 | 0.62 |
| KC3-X2-PH12 | 5.25 | 52 | 97.66 | 0.051 | 94.7 ± 0.29 | 15.20 | −0.61 |
| KC3-X1-PH12 | 5.25 | 52 | 114.1 | 0.044 | 88.2 ± 0.13 | 20.43 | 0.68 |
| KC3-X1-PH12 | 5.25 | 56 | 108.5 | 0.057 | 89.1 ± 0.43 | 13.16 | 0.72 |

The formulations ranged in size from approximately 97 to 117 nm in diameter The % entrapped mRNA ranged from 85-94% and LNPs exhibited positive surface charge at pH 5 ~+9 to +20 mV and a negative charge at pH 7 −1 to +1 mV.

TABLE 12A-2
Particle size (Z-ave), polydispersity (PDI) and mRNA encapsulation
efficiency (% EE) of LNPs that were lyophilized and reconstituted
(Post Lyo/R), made with either 52 or 56 mol % KC3-X1-PH12.

| | | | Post Lyo/R | | |
|---|---|---|---|---|---|
| ICL | N/P | mol % | Z-ave | PDI | % EE |
| KC3-X1-PH12 | 5.25 | 52 | 136.40 | 0.08 | 78.49 ± |
| | | | | | 1.07 |
| KC3-X1-PH12 | 5.25 | 56 | 140.20 | 0.02 | 75.46 ± |
| | | | | | 1.60 |

After lyophilization, it was found that the particle size increased by about 25 nm for each, and the % EE was reduced by about 10-12% points (compare to characteristics in Table 12-A-1).

To measure the ability of these mRNA-LNPs to induce antibodies, BALB/c mice were immunized at a high and 3-fold lower dose in the caudal thigh in a volume of 50 μL. The experimental design and dosing schema is shown in FIG. 20A. Twenty-one days post vaccination, serum was collected and stored at −80° C. until analysis. Mouse IgG endpoint antibody titers against the matching H1, H3, B-Victoria, RSVpreF or H5 antigens were determined by standard ELISA. For H1-specific Ab titers, we found for KC3-X1-PH12 that immunogenicity of freeze-dried material was not deleterious for biological activity (FIG. 20B and FIG. 20C). This was true for 52 and 56 mol % ICL with N/P=5.25, indicating that lyophilization may be an option for longer-term storage. Second, Ab titers were comparable between LNPs comprised of KC3-X1-PH12 and KC3-X2-PH12, showing that either orientation of the biodegradable ester bond can be used for PH12 with a KC3 headgroup. Third, we found for KC3-X1-PH12 that either 52 or 56 mol % with N/P=5.25 elicited similar immune responses; the same was true for X₂—PH12. For H3-specific Ab titers (FIG. 20D and FIG. 20E), results were similar to H1 titers: lyophilization of the mRNA-LPN was not deleterious to biological activity (H3 titers elicited by lyophilized material trended higher than for non-lyophilized, X₁—PH12 and X₂—PH12 were comparably immunogenic, and these ICLs can be used with either 52 or 56 mol % without impacting antibody titers. FIG. 20F and FIG. 20G show the B/Victoria Ab titers were similar KC3-X1-PH12 and KC3-X2-PH12.

Serum was collected from mice 3 weeks after i.m. vaccination and total anti-HA IgG Ab titers were determined for all mRNA-encoded antigens using a standard endpoint ELISA protocol. For H1-specific Ab titers, we found for KC3-X1-PH12 that immunogenicity of freeze-dried material was not deleterious for biological activity (FIGS. 20B and 20C). This was true for 52 and 56 mol % ICL with N/P=5.25, indicating that lyophilization may be an option for longer-term storage. Second, Ab titers were comparable between LNPs comprised of KC3-X1-PH12 and KC3-X2-PH12, showing that either orientation of the biodegradable ester bond can be used for PH12 with a KC3 headgroup. Third, we found for KC3-X1-PH12 that either 52 or 56 mol with N/P=5.25 elicited similar immune responses; the same was true for X2-PH12. For H1-3-specific Ab titers (FIGS. 20D and 20E) and B/Victoria-specific Ab titers (FIGS. 20F and 20G), results were similar to H1 titers: lyophilization of the mRNA-LPN was not deleterious to biological activity (H3 and B/Victoria titers elicited by lyophilized material trended higher than for non-lyophilized), X—PH12 and X2-PH12 were comparably immunogenic, and these CLs can be used with either 52 or 56 mol % without impacting antibody titers. In mice vaccinated with monovalent H5 mRNA (FIG. 20H), there was again no difference in Ab titers between X1-PH12 LNPs comprised of 52 or 56 mol %, showing that there is a mole range where KC3-X1-PH12 can be used in LNPs where immunogenicity is not compromised.

| SEQ | |
|---|---|
| ID NO | Description |
| 12 | A polypeptide sequence to A/Wisconsin/67/2022 H1. (GISAID: |
| | EPI_ISL_15928563) |
| 13 | Codon optimized RNA sequence encoding the polypeptide in SEQ ID NO: 12. |
| | The CDS is flanked at the 5′ and 3′ with HBB UTRs. |
| 14 | A polypeptide sequence to A/Darwin/6/2021 H3 (GISAID: EPI_ISL_2233238) |
| 914 | Codon optimized RNA sequence encoding the polypeptide in SEQ ID NO: 14. |
| | The CDS is flanked by the 5′ and 3′ HBB UTRs. |
| 915 | A polypeptide sequence to B/Austria/1359417/2021 (B/Victoria lineage)-like HA |
| | (GISAID: EPI_ISL_983345) |
| 17 | Codon optimized RNA sequence encoding the polypeptide in SEQ ID NO: 915. |
| | The CDS is flanked by the 5′ and 3′ HBB UTRs. |
| 9 | A polypeptide sequence to RSVpreF sc9-10, DS-Cav1, A146C-Y458C. PMID: |
| | 27478931 |
| 10 | Codon optimized RNA sequence encoding the polypeptide in SEQ ID NO: 9. The |
| | CDS is flanked by the 5′ and 3′ HBB UTRs. |

### Example 13. RSV mRNA Vaccines

### Example 13A. Comparing the Immunogenicity of RSVpreF mRNA-LNPs Comprised of KC3-X1-PH12 PS with Approved RSV Vaccines

The purpose of this RSVpreF immunogenicity study was to compare the KC3-X1-PH12/PS mRNA-LNP formulation with commercially approved preF vaccines: mRESVIA™ (Moderna, SM-102 LNP formulation), Arexvy™ (GSK, protein subunit, adjuvanted with AS01_{E}) and Abrysvo™ (Pfizer, protein subunit non-adjuvanted). BALB/c mice were dosed i.m. with either 0.3 or 0.1 ug of KC3-X1-PH12/PS mRNA-LNPs or mRESVIA. The physicochemical properties the KC3-X1-PH12/PS mRNA-LNPs are shown in Table 13A-1. Mice were also dosed i.m. with 4.8 ug Arexvy (preF protein, subtype A; contained 1 ug AS01_{E}) or 4.8 ug Abrysvo (2.4 ug subtype A preF, 2.4 ug subtype B preF). Total binding IgG endpoint titers 4 weeks post-prime are shown in FIGS. 28A/B. Mice were then boosted and endpoint titers 2 weeks post-boost are shown in FIGS. 28C/D. Note that the data points for Arexvy and Abrysvo are duplicated between A/B and C/D for comparison purposes and statistical analysis.

Vaccination with KC3-X1-PH12/PS elicited comparable or superior Ab titers to those of Arexvy, and both of these test articles were more immunogenic than mRESVIA (with the exception of post-boost titers at the 0.3 ug dose) and Abrysvo. Abrysvo and mRESVIA elicited the lowest Ab titers, and mRESVIA was only higher than Abrysvo post-boost at the 0.3 ug dose. In conclusion, the KC3-X1-PH12 PS-targeted LNP formulation is able to efficiently transfect antigen-presenting cells with mRNA encoding membrane RSVpreF antigen that results in potent B cell responses.

TABLE 13A-1
Physicochemical properties of mRNA-LNP formulations containing KC3-X1-
PH12, DSPC, DPPS, Chol, PEG-DMG (52/5/5/36.5/1.5 mol %, NP = 5.25) used in Example 13.
Particle size (Z-ave, nm), polydispersity (PDI), zeta potential at pH 5 and pH 7 and mRNA
encapsulation efficiency (% EE) are shown.

| | | | | % EE ± | Zeta | Zeta |
|---|---|---|---|---|---|---|
| ICL | mRNA | Z-Ave | PDI | stdev | pH 5 | pH 7 |
| KC3-X1-PH12 | RSVpreF. SEQ ID | 111.30 | 0.07 | 93.53 ± 0.21 | 22.67 | 0.3103 |
| | NO: 901 | | | | | |

### Example 13B. In Vivo Dose Response Comparison of RSVpreF mRNA-LNP Vaccines Comprised of KC3-X1-PH12 PS, KC3-X2-PH12 PS, or Clinical-Grade mRESVIA (Moderna)

The purpose of this study was to compare PS-targeted mRNA-LNPs comprised of the ICL KC3-X1-PH12 or the ICL KC3-X2-PH12, which has the ester linkage in the lipid tail in the reverse orientation relative to KC3-X1-PH12. Clinical-grade mRESVIA was used as a benchmark comparator. BALB/c mice were vaccinated i.m. with 0.3, 0.1, 0.03, or 0.01 ug preF mRNA and serum anti-RSVpreF IgG Ab titers were measured at 3 weeks. The physicochemical properties of the mRNA-LNPs are shown in Table 13B-1. KC3-X1-PH12 and KC3-X2-PH12 LNPs had similar properties.

No mice in the 0.01 ug dose group had titers greater than a 150-fold dilution, which was the lower limit of detection in this assay (data not shown) (FIGS. 29A, 29B and 29C). At the 0.03 ug dose level, all but one mouse in the mRESVIA group had detectable titers, and titers were similar between all three groups. The KC3-X1-PH12 and -X2-PH12 formulations were more immunogenic than mRESVIA at 0.1 and 0.3 ug. In conclusion, LNPs comprised of KC3-X1-PH12 and KC3-X2-PH12 were similarly immunogenic, and both were superior to mRESVIA, which uses the SM-102 ICL. Interestingly, the superior immunogenicity of the PH12 LNPs over mRESVIA was lost at 0.03 ug and lower. One potential explanation is the PH12 LNPs were nearing their lower immunogenicity thresholds and thus were not able to outperform mRESVIA.

TABLE 13B-1
Physicochemical properties of mRNA-LNP formulations containing
the ICLs KC3-X1-PH12 or KC3-X2-PH12 with DSPC, DPPS, Chol,
PEG-DMG (52/5/5/36.5/1.5 mol %, NP = 5.25) encapsulating
RSVpreF mRNA (SEQ ID NO: 901) used in the dose response
mouse study mouse in Example 13B. Particle size (Z-ave,
nm), polydispersity (PDI), zeta potential at pH 5 and pH
7 and mRNA encapsulation efficiency (% EE) are shown.

| | | | % EE ± | Zeta | Zeta |
|---|---|---|---|---|---|
| ICL | Z-Ave | PDI | stdev | pH 5 | pH 7 |
| KC3-X1-PH12 | 105.5 | 0.039 | 90.0 ± 0.08 | 24.00 | −4.58 |
| KC3-X2-PH12 | 104.3 | 0.045 | 91.2 ± 0.6 | 23.05 | −3.69 |

SEQ ID NO: 901 is a codon-optimized RNA sequence encoding a stabilized RSV prefusion F polypeptide. The CDS is flanked by 5′ and 3′ HBB UTRs.
SEQ ID NO: 902 is a codon-optimized RNA sequence encoding a stabilized RSV prefusion F polypeptide. The CDS is flanked by 5′ and 3′ HBB UTRs.
SEQ ID NO: 903 is a codon-optimized RNA sequence encoding a stabilized RSV prefusion F polypeptide. The CDS is flanked by 5′ and 3′ HBB UTRs.
SEQ ID NO: 904 is a codon-optimized RNA sequence encoding a stabilized RSV prefusion F polypeptide. The CDS is flanked by 5′ and 3′ HBB UTRs.

### Example 14. Trivalent Influenza mRNA Vaccines

One unique advantage of the mRNA vaccine platform is the ease and flexibility of co-encapsulating multiple mRNAs encoding different antigens. Seasonal influenza vaccines are a good example of this: each year seasonal influenza A H1N1 and H3N2 and influenza B/Victoria-like strains circulate, and due to constant mutations in the attachment hemagglutinin and virus reassortment, vaccine strains must be adjusted annually. There were two primary questions asked in this example. In this example, LNP optimization of the PEG lipid component was examined using trivalent influenza mRNA vaccines containing mRNAs encoding H1 (SEQ ID NO: 13), H3 (SEQ ID NO: 914), and B/Victoria (SEQ ID NO: 17). The study design is shown in FIG. 30A. BALB/c mice were injected i.m. with KC3-X1-PH12/PS mRNA-LNPs comprised with 1.5 mol % PEG-DMG (C14 lipid chain) or comprised with 1.5, 2, or 2.5 mol % PEG-DLG (C12 lipid chain). Three weeks after a single immunization, anti-HA IgG Ab titers were measured by standard endpoint ELISA for each HA subtype. The immunogenicity of these test articles were benchmarked against the ALC-0315 and SM-102 comparator formulations and against standard trivalent inactivated split-virus influenza vaccines manufactured by Seqirus. The physicochemical properties of the mRNA-LNP formulations are shown in Table 14A-1.

TABLE 14A-1
Physicochemical properties of mRNA-LNP formulations containing KC3-X1-
PH12, DSPC, DPPS, Chol, PEG-DMG (52/5/5/36.5/1.5 mol %, NP = 5.25) or KC3-X1-PH12,
DSPC, DPPS, Chol, PEG-DLG (52/5/5/38-x/x mol %, NP = 5.25) where x is listed as the mol %
PEG-Lipid in Table 11A-1 and in used in Example 14. The SM-102 based formulation consisted
of SM-102, DSPC, Chol, PEG-DMG (50/10/38.5/1.5 mol %) with a NP of 5 and the ALC-0315
based formulation consisted of ALC-0315, DSPC, Chol, ALC-0159 (46.3/9.4/42.9/1.56 mol %)
with an NP of 6.2. Particle size (Z-ave, nm), polydispersity (PDI), zeta potential at pH 7 and pH 5
and mRNA encapsulation efficiency (% EE) are shown.

| | PEG-Lipid, | | | % EE ± | Zeta | Zeta |
|---|---|---|---|---|---|---|
| ICL | mol % | Z-Ave | PDI | stdev | pH 5 | pH 7 |
| KC3-X1-PH12 | DMG, 1.5 | 104.3 | 0.058 | 90.0 ± 0.6 | 24.51 | −2.81 |
| KC3-X1-PH12 | DLG, 1.5 | 112.3 | 0.069 | 90.3 ± 0.3 | 23.13 | −1.08 |
| KC3-X1-PH12 | DLG, 2.0 | 105.5 | 0.107 | 91.7 ± 0.2 | 20.07 | −1.53 |
| KC3-X1-PH12 | DLG, 2.5 | 98.4 | 0.122 | 92.0 ± 0.5 | 18.12 | −2.55 |
| ALC-0315 | ALC-0159, 1.56 | 81.2 | 0.105 | 80.9 ± 0.5 | 8.00 | −13.53 |

### Example 14A. Immunogenicity Comparison of KC3-X1-PH12 PS mRNA-LNPs Comprised with PEG-DMG or Various Mol % of PEG-DLG

The primary objective here was to determine whether PEG-DLG could be used in place of PEG-DMG. While PEG-DMG is the most common form used in LNPs, there may be advantages to using PEG-DLG instead: since PEG-DLG has a shorter lipid tail (C12), it should dissociate faster from the LNP after injection. This could decrease the ability of PEG to cross-link B cell receptors and decrease the chance of developing anti-PEG Abs and potentially reduce vaccine reactogenicity.

FIGS. 30B-30D show Ab titers for H1, FIGS. 30E-30G show Ab titers for H3, and FIGS. 30H-J show Ab titers for B/Victoria. For all HA antigens, IgG titers were comparable between the KC3-X1-PH12/PS compositions with 1.5 mol % of either PEG-DMG or PEG-DLG. For all HA subtypes at the lowest 0.03 ug dose, LNPs comprised of 2.0 mol % PEG-DLG had significantly lower titers, indicating that this mol % decreased the immunogenicity of the mRNA-LNP formulation; this was only evident at low doses when Ag levels were limiting. Titers for the 2.5 mol % PEG-DLG were only significantly reduced relative to 1.5 mol % PEG-DMG for H1 at 0.03 ug. In conclusion, the KC3-X1-PH12/PS LNP formulation with PEG-DLG at 1.5 mol % is just as immunogenic as PEG-DMG at 1.5 mol %. PH12/PS LNPs comprised with PEG-DLG also had similar physicochemical properties to those of PEG-DMG LNPs, indicating they formed “well-behaved” LNPs and there was no obvious deleterious effect of switching from DMG to DLG.

### Example 14B. Immunogenicity Comparison of the KC3-X1-PH12 PS LNP Formulation with the SM-102 and ALC-0315 LNP Formulations and Clinical-Grade Flu Vaccine Comparators

The primary objective here was to compare the immunogenicity of LNPs formulated with KC3-X1-PH12/PS PEG-DMG (1.5 mol %) with the immunogenicity of comparator LNP formulations using the SM-102 or ALC-0315 ICLs and to benchmark their immunogenicity to standard inactivated split-virus trivalent seasonal influenza vaccine. We used trivalent egg-based (Afluria™) or cell-based (Flucelvax™) vaccines manufactured by Seqirus. These vaccines and the mRNA test articles use the same H1 and B/Victoria HA antigens, but it is important to note these Seqirus vaccines use updated influenza A/H3N2 strains than what is encoded by mRNA that differ by 12-13 amino acids.

FIGS. 31A-31C show total anti-H1 Ab titers, FIGS. 31D-31F show total anti-H3 Ab titers, and FIGS. 31G-31I shows total anti-B/Victoria Ab titers. Note that the data points for Afluria and Flucelvax are duplicated between all plots for each HA subtype—this was done for statistical comparison purposes. The KC3-X1-PH12/PS PEG-DMG mRNA-LNP generally elicited the highest IgG binding Abs at the 0.03 ug dose compared to all other formats. As the dose increased, Ab titers between the mRNA vaccines became similar, which is what we have observed for other antigens—as Ag levels increase in the draining LNs, differences in LNP delivery/transfection efficiency are no longer a limiting factor and Ab titers converge to a similar level. In almost all cases Ab titers elicited by the KC3-X1-PH12/PS formulation induced higher levels than either Afluria or Flucelvax. In conclusion, the KC3-X1-PH12/PS LNP platform can be used to encapsulate multiple mRNAs encoding vaccine antigens. This formulation generates B cell responses more efficiently than the comparator SM-102 and ALC-0315 mRNA-LNP formulations, and this is most evident at low dose levels when antigen expression becomes a limiting factor. It is also far more efficient than traditional multivalent split-virus vaccines. This is particularly important in pandemic situations where people do not have pre-existing immunity.

SEQ ID NO: 905 is a polypeptide HA sequence from the influenza vaccine strain A/Victoria/4897/2022 (HIN1)pdm09. (GISAID: EPI_ISL_16714268)

SEQ ID NO: 906 is a polypeptide HA sequence from the influenza vaccine strain A/District of Columbia/27/2023 (H3N2). (GISAID: EPI_ISL_18937823)

SEQ ID NO: 907 is a polypeptide HA sequence from the influenza strain A/Croatia/10136RV/2023 (H3N2). (GISAID: EPI_ISL_19296516)

### Example 15. Design of a Dengue Virus (DENV) mRNA Vaccine Encoding Non-Structural T Cell Antigens

There are four dengue virus (DENV) serotypes, which are defined by a lack of cross-neutralizing Abs and cross-protection. While initial exposure to DENV is generally mild, severe disease can be caused by secondary exposure to a different serotype, and this is believed to be caused by antibody-dependent enhancement caused by cross-reactive but poorly neutralizing Abs. Consequently, a broadly efficacious and safe DENV vaccine likely needs to elicit balanced nAbs across all four serotypes. In addition to serotype-specific humoral immunity, T cells are able to provide complimentary cross-serotype protection by recognizing conserved non-structural (NS) proteins not accessible to antibodies. mRNA vaccines are able to elicit robust T cell responses, and peptide presentation to T cells can be enhanced by targeting mRNA-encoded antigen to endosomal and lysosomal compartments. For example, regions of NS3, NS4B and NS5 can be concatenated into a single CDS and flanked with sequences encoding the human LAMP-1 SP (5′ end) and LAMP-1 terminal domain (3′ end). This mRNA construct could be encapsulated into KC3-X1-PH12/PS LNPs and developed into a vaccine.

### Example 16. Design of a Malaria mRNA Vaccine

After human infection following a blood meal by a mosquito, sporozoites infect hepatocytes and initiate liver-stage infection. This transitions into the erythrocytic stage where merozoites asexually reproduce in red blood cells. Protein antigens specific to each stage in the parasite life cycle can be targeted by the adaptive immune system. Two approved liver-stage vaccines, RTS,S and R₂₁ target the liver stage by eliciting B and T cell responses against the circumsporozoite protein (CSP). Other vaccine candidates in development target blood stage antigens with the goal of eliciting neutralizing Abs that prevent infection of red blood cells. Merozoite antigens of interest include PfRH5 and PfMSP10. A multivalent mRNA-LNP vaccine could be developed that targets both liver and blood stage antigens. For example, a trivalent vaccine could include mRNAs encoding the conserved N-terminal region of MSP10 (SEQ ID NO: 911), RH5 (SEQ ID NO: 912), and either the CSP antigens contained within the R₂₁ or RTS,S vaccines. These mRNAs could be co-encapsulated into KC3-X1-PH12/PS LNPs.

SEQ ID NO: 911 is a polypeptide sequence of a Plasmodium falciparum MSP10 protein ectodomain, amino acids 1-188. UniProt Q868U4_PLAFA. SEQ ID NO: 912 is a polypeptide sequence of a Plasmodium falciparum RH5 protein (version RH5.1ANLC). Deglycosylation mutations T40A, T216A, T286A, T299A. Removal of an N-terminus domain, a disordered intrinsic loop, and a C-terminus domain.

### Example 17. Evaluation of Physico-Chemical Properties and Targeted Transfection Activity of the LNPs Prepared from ICLs with Various Headgroups and Hydrolysable Tail Moieties

A series of LNP comprising ICLs with various basic nitrogen headgroups and hydrocarbon lipid tails having a hydrolysable ester group and comprising a branched hydrocarbon moiety (Table 17A) were prepared according to Example 3 using the lipid composition of 52 mol % ICL, 5 mol % DSPC, 5 mol % DPPS, 36.5 mol % Cholesterol, 1.5 mol % PEG-DMG, and firefly luciferase (Luc) encoding mRNA (SEQ ID 3) at the mRNA-lipid N/P ratio of 5.25. Specifically, the lipid components dissolved in 100% ethanol were combined in necessary proportions, the lipid solution was passed through 0.2-μm Nylon sterile filter and mixed in the flow with 33.5 μg/ml solution of mRNA in 6.25 mM sodium acetate buffer pH 4.93 at the volume ratio of 1:3 using a microfluidic mixer (ChipShop microfluidics chip 187) and the total flow rate of 18 ml/min. The mixture was immediately diluted with equal volume of ice-cold PBS, passed through a 0.2-μm PES filter, and subjected to several cycles of the buffer exchange by centrifugal concentration-dilution using Amicon Ultra RC concentrator first with PBS (to estimated residual ethanol of less than 2%), then with 10 mM Tris-HCl, 10% sucrose, pH 7.4 (to estimated residual NaCl of less than 0.5 mM). At the last centrifugation LNP preparations were concentrated to the estimated 40-60 μg/ml mRNA, filter-sterilized by passage through 0.2-μm PES filter, vialed, and frozen at −80C for storage. The LNP preparations were thawed by brief immersion in water at room temperature with gentle shaking and further analyzed for particle size, percent mRNA encapsulation, total mRNA concentration, particle zeta-potential according to Example 4, and for mRNA transfection activity by luciferase expression in the cultured murine dendritic cells at the mRNA concentrations of 0.3 μg/ml and 0.1 μg/ml according to Example 6A (24 hour incubation). The results are given in Table 17B and Table 17C. LNP formulation containing ICL KC3-X1-PH12 was prepared earlier using identical protocol and used as a transfection activity reference analyzed side-by-side with the above formulations.

TABLE 17A
Structures of ICL used in preparation of LNPs of Example 17.

| ICL | Headgroup | R1, R2 |
|---|---|---|
| SM-X1-PH12B | 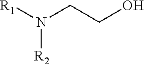 | 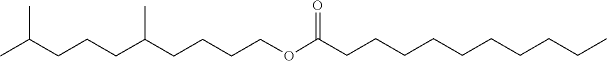 |
| DODAP-X1-PH5 | 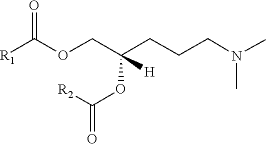 | 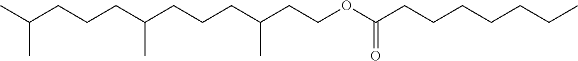 |
| ALC-PHY12A | 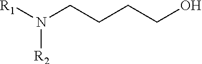 | 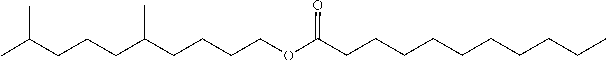 |
| DODAP-X1-PH12 | 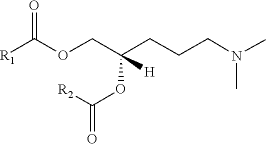 | 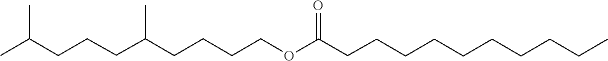 |
| KC3-X1-PH12 | 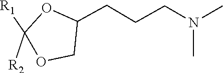 | 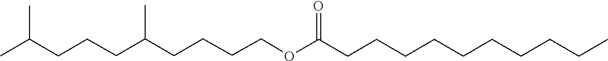 |
| MA-X1-PH12 | 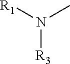 | 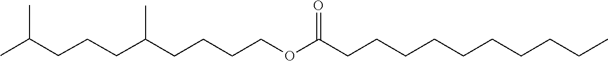 |
| MA-X2-PH12 | 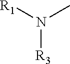 | 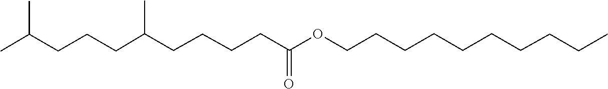 |
| MAE-X1-PH12 | 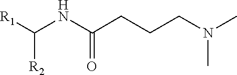 | 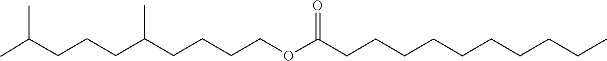 |
| MAE-X2-PH12 | 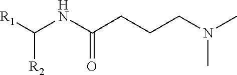 | 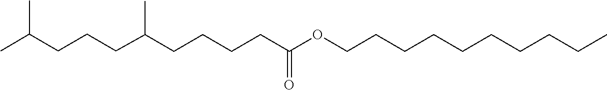 |
| DODMA-X1-PH5 | 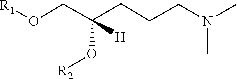 | 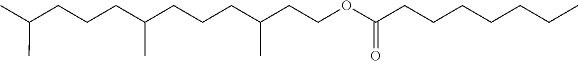 |
| KC2-X1-PH12 | 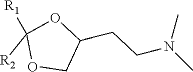 | 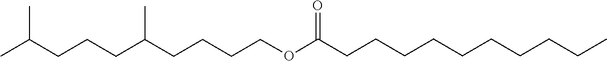 |
| DMA3E-X1-PH12 | 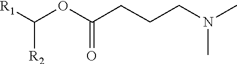 | 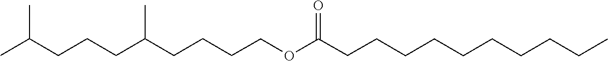 |
| DMA3E-X2-PH12 | 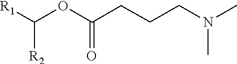 | 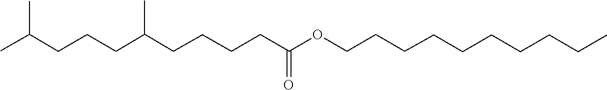 |
| DMA4E-X1-PH12 | 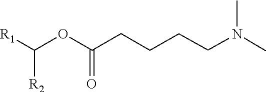 | 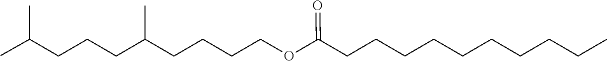 |
| SM-X1-PH12A | 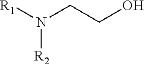 | 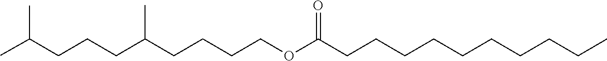 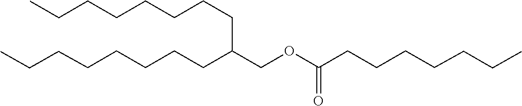 |

TABLE 17-B
Properties of LNPs prepared with ICLs of Table 17-A. Abbreviations:
Xz—z-average particle size; PDI—polydispersity index;
% EE—encapsulation efficiency after freezing and thawing,
%; Zeta—particle zeta-potential. NA—Not available: LNPs
were not obtained due to extremely poor Amicon diafiltration rate.

| | | | | Zeta, mv | Zeta, mV (pH |
|---|---|---|---|---|---|
| ICL | Xz, nm | PDI | % EE | (pH 5) | 7) |
| MA-X1-PH12 | 78.9 | 0.076 | 88.36 | 9.90 | −11.06 |
| MA-X2-PH12 | 80.5 | 0.063 | 88.77 | 8.76 | −12.10 |
| MAE-X1-PH12 | NA | | | | |
| MAE-X2-PH12 | NA | | | | |
| DODMA-X1-PH5 | 133.5 | 0.10 | 97.16 | 27.01 | −1.11 |
| KC2-X1-PH12 | 96.4 | 0.065 | 91.70 | 14.68 | −8.89 |
| DMA3E-X1-PH12 | 89.5 | 0.020 | 94.34 | 13.17 | −10.63 |
| DMA3E-X2-PH12 | 85.4 | 0.042 | 95.09 | 13.76 | −8.63 |
| DMA4E-X1-PH12 | 94.6 | 0.045 | 95.73 | 18.01 | −7.78 |
| SM-X1-PH12A | 73.8 | 0.058 | 94.88 | 7.93 | −6.40 |
| SM-X1-PH12B | 89.7 | 0.083 | 91.33 | 15.44 | −7.36 |
| DODAP-X1-PH5 | 170.2 | 0.168 | 93.72 | 26.37 | −0.50 |
| ALC-PHY12A | 108.0 | 0.053 | 82.27 | 18.85 | −9.18 |
| DODAP-X1-PH12 | 105.3 | 0.051 | 91.68 | 23.77 | −5.24 |

TABLE 17-C
Luciferase expression (Luc) by MutuDC1940
cells incubated with the LNP of Example 17.

| | 0.3 μg/ml | | | |
|---|---|---|---|---|
| LNP mRNA | | Luc, % of | 0.1 μg/ml | |
| concentration | Luc, | KC3-X1- | Luc, | Luc, % of |
| ICL | ng/10⁶ cells | PH12 | ng/10⁶ cells | KC3-X1-PH12 |
| MA-X1-PH12 | 12.57 ± 1.82 | 4.1 | 1.98 ± 0.2 | 1.9 |
| MA-X2-PH12 | 21.95 ± 1.92 | 7.1 | 2.72 ± 0.41 | 2.6 |
| DODMA-X1-PH5 | 119.61 ± 8.62 | 38.9 | 35.42 ± 2.85 | 33.3 |
| KC2-X1-PH12 | 171.8 ± 10.17 | 55.9 | 54.33 ± 4.13 | 51.1 |
| DMA3E-X1-PH12 | 83.34 ± 9.22 | 27.1 | 16.37 ± 1.24 | 15.4 |
| DMA3E-X2-PH12 | 74.12 ± 5.32 | 24.1 | 15.13 ± 0.92 | 14.2 |
| DMA4E-X1-PH12 | 240.95 ± 8.86 | 78.4 | 89.86 ± 6.34 | 84.4 |
| SM-X1-PH12A | 245.97 ± 6.02 | 80.0 | 79.74 ± 2.77 | 74.9 |
| SM-X1-PH12B | 152.89 ± 12.53 | 49.7 | 49.35 ± 3.13 | 46.4 |
| DODAP-X1-PH5 | 64.15 ± 4.18 | 20.9 | 11.49 ± 1.15 | 10.8 |
| ALC-PHY12A | 152 ± 11.88 | 49.4 | 42.53 ± 3.46 | 40.0 |
| DODAP-X1-PH12 | 262.02 ± 15.92 | 85.2 | 87.12 ± 5.44 | 81.9 |
| KC3-X1-PH12 | 307.49 ± 10.14 | 100.0 | 106.41 ± 9.32 | 100.0 |

Among the various studied ICL headgroups combined with PH5 or PH12 lipophilic tails carrying a carboxylic ester bond in X1 (*—O—C(═O))—**) or X2 (*—C(═O)—O—**) configuration the ability to form LNPs and the level of PS-targeted transfection and expression of LNP-encapsulated mRNA was variable and hard to predict from the headgroup structure. Interaction of mRNA with the ICLs having a basic nitrogen atom linked to the tail portion via an amide bond and C3 hydrocarbon spacer (MAE configuration) resulted in the complexes that prevented diafiltration through the Amicon RC membrane and therefore the LNPs could not be fully formed, while structurally close variants (DMA3E) with an ester bond instead of amide, did not impede diafiltration and effectively formed LNPs with small particle size and mRNA encapsulation. However the particle size and mRNA encapsulation efficiently were not generally predictive for the high targeted expression of encapsulated mRNA in the target-competent dendritic cell line. Thus, two ICLs with the simple tertiary amine headgroup (MA configuration) linked to the PH12 lipophilic chains identical to that of the reference ICL KC3-X1-PH12 and its analog KC3-X2-PH12, both highly active in mRNA expression, effectively formed LNPs with small size and high encapsulation efficiency, but showed poor expression. Other types of headgroups, in particular with DODAP, DMA4E, and SM headgroups linked to PH12 tails (the latter with an asymmetric tail structure) unpredictably produced ICLs with the levels of mRNA expression comparable to that of the reference ICL (78-85%), however on the sum of salient properties of the formed LNPs, neither headgroup structure with the studied tails exceeded KC3-X1-PH12. In a side-by-side comparison, PH5 tails resulted in lower expression of mRNA compared to the PH12 tails linked to the same headgroup (e.g., DODAP-X1-PH5 at 20.9% vs. DODAP-X1-PH12 at 85.2%). The KC2 headgroup linked to X1-PH12 tails resulted in about two-fold decrease in the expression of encapsulated mRNA compared to KC3 headgroup, as was also previously observed with OA (non-hydrolysable) tails.

### Example 18. Exemplary Phosphatidyl-L-Serine and Phosphatidylglycerol Targeted LNP Formulations Containing Hydrolysable KC3-, KC2-Phytyl or Extended Length Linear Biodegradable Akyl ICLs

Additional formulations shown below could also be prepared using the methods described in the Examples above. All lipid concentrations are shown as mol % as a percentage of the total lipid in the LNP. The below compositions vary in conjugated lipid content from 1.5 to 4 mol 0, in sterol content from 25-43 mol 00 in ICL content from 46-58 mol 00 in saturated phosphatidyl-L-serine or phosphatidylglycerol content from 2.5-7.5 mol 00 and in total noncationic phospholipid content from 2.5-15 mol 00 Most compositions would contain two phospholipids, typically phosphatidyl-L-serine (DSPS or DPPS being preferred) or phosphatidylglycerol (e.g. DSPG) and phosphatidylcholine, although some exemplary formulations may contain more than two phospholipids. LNPs may contain mRNA or other nucleic acid at N/P of 4-7.

TABLE 18-A
Exemplary LNP formulations

| | | | Additional | | Conjugated |
|---|---|---|---|---|---|
| Formulation | Ionizable Cationic | | Phospholipid(s) | Sterol | lipid |
| (#) | Lipid (mol %) | PS (mol %) | (mol %) | (mol %) | (mol %) |
| 1 | ICL (48) | DPPS (5) | DSPC (5) | Chol (40.5) | PEG-DMG (1.5) |
| 2 | ICL (48) | DSPG (5) | DSPC (5) | Chol (40.5) | PEG-DMG (1.5) |
| 3 | ICL (48) | DPPS (3.5) | DSPC (6.5) | Chol (40.5) | PEG-DMG (1.5) |
| 4 | ICL (48) | DSPG (3.5) | DSPC (6.5) | Chol (40.5) | PEG-DMG (1.5) |
| 5 | ICL (48) | DPPS (5) | DSPC (5) | Chol (39.5) | PEG-DLG (2.5) |
| 6 | ICL (48) | DSPG (5) | DSPC (5) | Chol (39.5) | PEG-DLG (2.5) |
| 7 | ICL (48) | DPPS (3.5) | DSPC (6.5) | Chol (39.5) | PEG-DLG (2.5) |
| 8 | ICL (48) | DSPG (3.5) | DSPC (6.5) | Chol (39.5) | PEG-DLG (2.5) |
| 9 | ICL (48) | DPPS (5) | DSPC (7.5) | Chol (38.5) | PEG-DMG (1.5) |
| 10 | ICL (48) | DSPG (5) | DSPC (7.5) | Chol (38.5) | PEG-DMG (1.5) |
| 11 | ICL (50) | DPPS (5) | DSPC (5) | Chol (38.5) | PEG-DMG (1.5) |
| 12 | ICL (50) | DSPG (5) | DSPC (5) | Chol (38.5) | PEG-DMG (1.5) |
| 13 | ICL (52) | DPPS (5) | DSPC (5) | Chol (36.5) | PEG-DMG (1.5) |
| 14 | ICL (52) | DSPG (5) | DSPC (5) | Chol (36.5) | PEG-DMG (1.5) |
| 15 | ICL (54) | DPPS (5) | DSPC (5) | Chol (34.5) | PEG-DMG (1.5) |
| 16 | ICL (54) | DSPG (5) | DSPC (5) | Chol (34.5) | PEG-DMG (1.5) |
| 17 | ICL (52) | DPPS (5) | DSPC (5) | Chol (36.5) | PEG-DLG (3.5) |
| 18 | ICL (52) | DSPG (5) | DSPC (5) | Chol (36.5) | PEG-DLG (3.5) |
| 19 | ICL (48) | DPPS (5) | DSPC (10) | Chol (36) | PEG-DMG (1.5) |
| 20 | ICL (48) | DSPG (5) | DSPC (10) | Chol (36) | PEG-DMG (1.5) |
| 21 | ICL (47.5) | DSPS (5) | DSPC (5) | β-sitosterol (41) | PEG-DMG (1.5) |
| 22 | ICL (50) | DSPS (5) | DSPC (5) | β-sitosterol | PEG-DMG (1.5) |
| | | | | (38.5) | |
| 23 | ICL (52.5) | DSPS (5) | DSPC (5) | β-sitosterol | PEG-DMG (1.5) |
| | | | | (38.5) | |
| 24 | ICL (55) | DSPS (5) | DSPC (5) | β-sitosterol | PEG-DMG (1.5) |
| | | | | (38.5) | |
| 25 | ICL (46) | DPPS (3.5) | DSPC (6.5) | Chol (42.5) | PEG-DMG (1.5) |
| 26 | ICL (46) | DSPG (3.5) | DSPC (6.5) | Chol (42.5) | PEG-DMG (1.5) |
| 27 | ICL (48) | DSPS (5) | DSPC (5) | Chol (40.5) | PEG-DMG (1.5) |
| 28 | ICL (48) | DSPS (3.5) | DSPC (6.5) | Chol (40.5) | PEG-DMG (1.5) |
| 29 | ICL (48) | DSPS (5) | DSPC (5) | Chol (399) | PEG-DMG (1) |
| 30 | ICL (48) | DSPS (5) | DSPC (5) | Chol (38.5) | PEG-DMG (0.5) |
| 31 | ICL (48) | DPPS (5) | DPPC (5) | Chol (40.5) | PEG-DMG (1.5) |
| 32 | ICL (48) | DPPS (5) | DPPC (5) | Chol (39.5) | PEG-DLG (2.5) |
| 33 | ICL (48) | DPPS (5) | HSPC (5) | Chol (40.5) | PEG-DMG (1.5) |
| 34 | ICL (48) | DPPS (5) | EggSM (5) | Chol (40.5) | PEG-DMG (1.5) |
| 35 | ICL (48) | — | DSPC (10) | Chol (39.5) | PEG-DLG (2.5) |
| 36 | ICL (50) | — | DSPC (10) | Chol (37.5) | PEG-DLG (2.5) |
| 37 | ICL (56) | DPPS (5) | DSPC (7.5) | Chol (30) | PEG-DMG (1.5) |
| 38 | ICL (56) | DSPG (5) | DSPC (5) | Chol (32.5) | PEG-DMG (1.5) |
| 39 | ICL (56) | DPPS (5) | DSPC (5) | Chol (30.5) | PEG-DLG (1.5) |
| 40 | ICL (56) | DSPG (5) | DSPC (5) | Chol (32.5) | PEG-DLG (1.5) |
| 41 | ICL (58) | DPPS (5) | DSPC (5) | Chol (30.5) | PEG-DMG (1.5) |
| 42 | ICL (58) | DPPS (3.5) | DSPC (6.5) | Chol (30.5) | PEG-DMG (1.5) |

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

All publications, patents and patent applications referenced in this specification are incorporated herein by reference in their entirety for all purposes to the same extent as if each individual publication, patent or patent application were specifically indicated to be so incorporated by reference.

What is claimed is:

1. A lipid nanoparticle (LNP) composition comprising a mRNA nucleic acid encapsulated in lipidic nanoparticles comprising:
a. an ionizable cationic lipid of formula (I) at a N/P ratio of 3.5 to 8 relative to the mRNA nucleic acid in a total amount of 40-65 mol % of the total lipid content of the LNP composition: 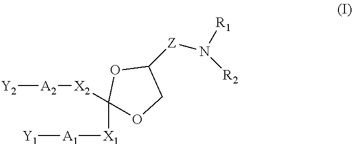
wherein
R₁ and R₂ are each independently C₁₋₄ alkyl optionally substituted with hydroxyl;
Z is *—(CH₂)ₙ—**, wherein * is a covalent bond to the 1,3-dioxolane ring and ** is a covalent bond to —NR₁R₂, and n is 3, 2 or 4;
—X₁— and —X₂— are the same are each independently —(CH₂)₍ₓ₎—, wherein (x) is 6-10;
-A₁- and -A₂- are the same and are each independently ester groups;
—Y₁ and —Y₂ are the same and are each independently aliphatic chains having a total of (y) carbon atoms each, wherein (y) is 10-20; and
wherein (i) the sum of (x)+(y) is 21-25; or (x) is 8-10 and (x)+(y) is 18-25; or (y) is 19-20; and
b. one or more phospholipids in a total amount of phospholipids of 5-25 mol % of the total lipid content of the LNP composition;
c. a PEG-containing conjugated lipid in a total amount of 0.5-5.0 mol % of the total lipid content of the LNP composition; and
d. a sterol in a total amount of the remaining 25-45 mol % of the total lipid content of the LNP composition.

2. The composition of claim 1, wherein the LNP composition comprises two or more phospholipids comprising:
a. an anionic phospholipid consisting of a phosphatidyl-L-serine (PS) or a phosphoglycerol (PG) anionic phospholipid, in a total amount of X mol % of the total lipid content of the LNP composition, wherein X is 2.5 mol % to 7.5 mol %; and
b. a neutral phospholipid consisting of a phospholipid selected from the group consisting of: a phosphocholine, a phosphatidylcholine and a sphingomyelin (SM), in a total amount of (10-X) mol % of the total lipid content of the LNP composition.

3. The composition of claim 2, wherein the anionic phospholipid is selected from the group consisting of: dipalmitoylphosphatidyl-L-serine (DPPS), distearoylphosphatidyl-L-serine (DSPS) and (1,2-Distearoyl-sn-glycero-3-phosphoglycerol (DSPG).

4. The composition of claim 2, wherein the neutral phospholipid is selected from the group consisting of: distearoylphosphatidylcholine (DSPC), hydrogenated soy phosphatidylcholine (HSPC), dipalmitoylphosphatidylcholine (DPPC), and sphingomyelin (SM).

5. The composition of claim 2, wherein the two or more phospholipids consist of dipalmitoylphosphatidyl-L-serine (DPPS) anionic phospholipid and distearoylphosphatidylcholine (DSPC) neutral lipid, and wherein the sterol is cholesterol.

6. The composition of claim 5, wherein the composition comprises the anionic phospholipid in a total amount of 5 mol % of the total lipid content of the LNP composition; and the neutral phospholipid in a total amount of 5 mol % of the total lipid content of the LNP composition.

7. The composition of claim 1, wherein the composition comprises a PEG(2000)-conjugated lipid in a total amount of 1.5 mol % of the total lipid content of the LNP composition.

8. The composition of claim 7, wherein the PEG(2000)-conjugated lipid is PEG(2000)-dimyristoylglycerol (PEG-DMG) in a total amount of 1.5 mol % of the total lipid content of the LNP composition.

9. The composition of claim 6, wherein the composition comprises PEG(2000)-dimyristoylglycerol (PEG-DMG) as the PEG-containing conjugated lipid in a total amount of 1.5 mol % of the total lipid content of the LNP composition.

10. The composition of claim 1, comprising the ionizable cationic lipid of formula (I) at a N/P ratio of 5 to 6 relative to the mRNA nucleic acid in a total amount of 48-56 mol % of the total lipid content of the LNP composition.

11. The composition of claim 10, wherein the sterol is cholesterol and the two or more phospholipids consist of:
a. dipalmitoylphosphatidyl-L-serine (DPPS) in a total amount of 5 mol % of the total lipid content of the LNP composition; and
b. distearoylphosphatidylcholine (DSPC) in a total of 5 mol % of the LNP composition.

12. The composition of claim 11, wherein the PEG-containing conjugated lipid is PEG(2000)-dimyristoylglycerol (PEG-DMG) in a total amount of 1.5 mol % of the total lipid content of the LNP composition.

13. The composition of claim 1, wherein the ionizable cationic lipid a compound of Formula (I), wherein Y₁ and Y₂ each independently comprises 2, 3 or 4 isopentane (IPN) or 2-methylbut-2-ene (2 MB) monomer units.

14. The composition of claim 13, wherein n is 3.

15. The composition of claim 14, wherein R₁ and R₂ are each methyl.

16. The composition of claim 15, wherein the LNP composition comprises a mRNA nucleic acid encapsulated in lipidic nanoparticles comprising:
a. the ionizable cationic lipid at a N/P ratio of 5 to 6 relative to the mRNA nucleic acid and in a total amount of 52-56 mol % of the total lipid content of the LNP composition;
b. 1,2-distearoyl-sn-glycero-3-phosphocholine (DSPC) in a total amount of phospholipids of 5 mol % of the total lipid content of the LNP composition;
c. a dipalmitoylphosphatidyl-L-serine (DPPS) in a total amount of phospholipids of 5 mol % of the total lipid content of the LNP composition;
d. PEG(2000)-dimyristoylglycerol (PEG-DMG) in a total amount of 1.5 mol % of the total lipid content of the LNP composition; and
e. the sterol is cholesterol in a total amount of up to 32.5-36.5 mol % of the total lipid content of the LNP composition.

17. The composition of claim 16, wherein Y₁ and Y₂ each independently comprises 2, 3 or 4 2-methylbut-2-ene (2 MB) monomer units.

18. The composition of claim 17, wherein the ionizable cationic lipid compound is KC3-X1-PH12 or KC3-X2-PH12:

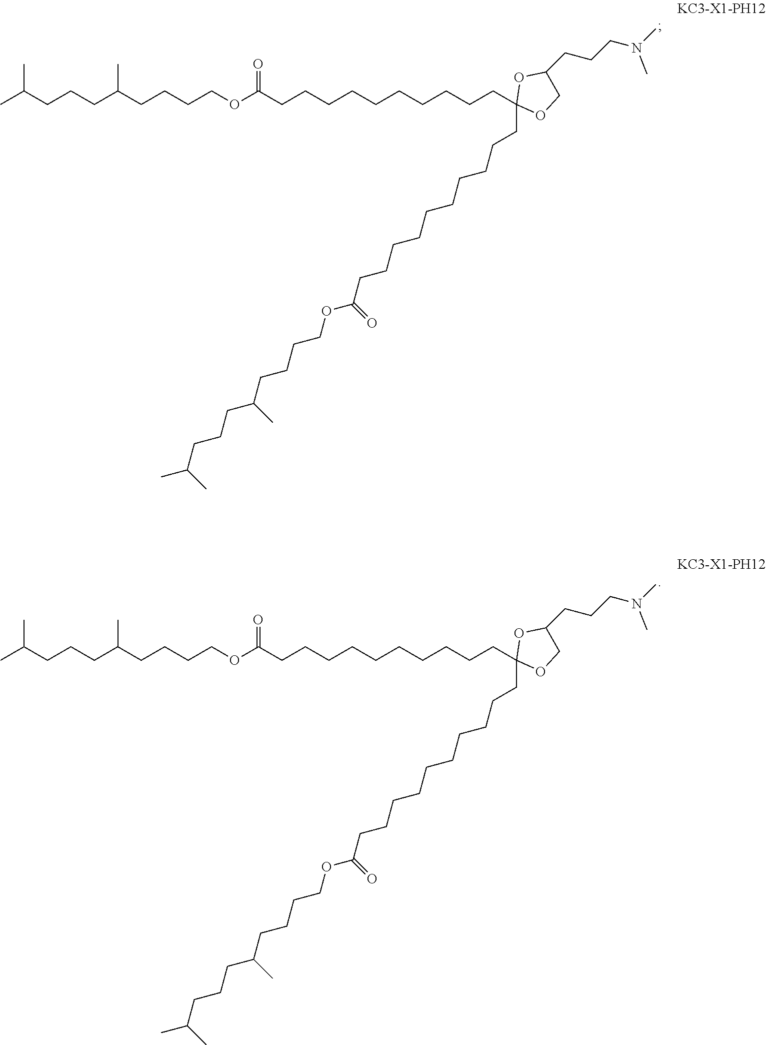

19. The composition of claim 18, comprising a mRNA nucleic acid encapsulated in lipidic nanoparticles consisting of:
a. a KC3-X1-PH12 ionizable cationic lipid 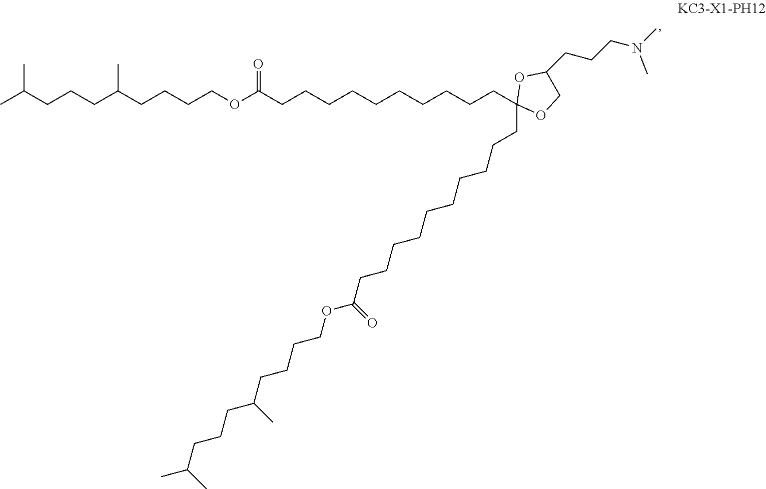 [SMILES: CC(C)CCCC(C)CCCCOC(=O)CCCCCCCCCCC1(CCCCCCCCCCC(=O)OCCCCC(C)CCCC(C)C)OCC(CCCN(C)C)O1]
at a N/P ratio of 5.25 relative to the mRNA nucleic acid in a total amount of 52 mol % of the total lipid content of the LNP composition;
b. 1,2-distearoyl-sn-glycero-3-phosphocholine (DSPC) in a total amount of phospholipids of 5 mol % of the total lipid content of the LNP composition;
c. dipalmitoylphosphatidyl-L-serine (DPPS) in a total amount of phospholipids of 5 mol % of the total lipid content of the LNP composition;
d. PEG(2000)-dimyristoylglycerol (PEG-DMG) in a total amount of 1.5 mol % of the total lipid content of the LNP composition; and
e. cholesterol in a total amount of 36.5 mol % of the total lipid content of the LNP composition.

20. A lipid nanoparticle (LNP) vaccine composition, comprising a mRNA nucleic acid encoding one or more immunogenic proteins, encapsulated in lipidic nanoparticles consisting of:
a. an ionizable cationic lipid of Formula (I) at a N/P ratio of 4-6 relative to the mRNA nucleic acid, 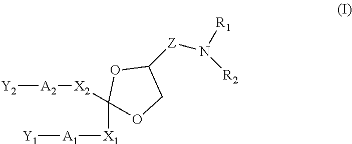
or a pharmaceutically acceptable salt thereof, wherein
R₁ and R₂ are each methyl;
Z is *—(CH₂)ₙ—**, wherein * is a covalent bond to the 1,3-dioxolane ring and ** is a covalent bond to —NR₁R₂, and n is 3, 2 or 4;
—X₁— and —X₂— are the same and are each independently —(CH₂)₍ₓ₎—, wherein (x) is 6-10;
-A₁- and -A₂- are the same or different and are each independently ester groups;
—Y₁ and —Y₂ are the same and are each aliphatic chains having a total of (y) carbon atoms each and comprising one or more isopentane (IPN) or 2-methylbut-2-ene (2 MB) monomer units, wherein (y) is 10-20, and wherein the sum of (x)+(y) is 16-25;
b. an anionic phospholipid consisting of a phosphatidyl-L-serine (PS) or a phosphoglycerol (PG) anionic phospholipid in a total amount of X mol % of the total lipid content of the LNP composition, wherein X is 2.5-7.5;
c. a neutral phospholipid consisting of a phospholipid selected from the group consisting of: a phosphocholine, a phosphatidylcholine and a sphingomyelin (SM), in a total amount of phospholipids of (10-X) mol % of the total lipid content of the LNP composition;
d. a PEG-containing conjugated lipid in a total amount of 0.5-2.5 mol % of the total lipid content of the LNP composition; and
e. cholesterol in a total amount of the remaining mol % of the total lipid content of the LNP composition.
